(19) United States
(12) Patent Application Publication
(10) Pub. No.: US 2024/0093311 A1
(43) Pub. Date: Mar. 21, 2024

(54) BREAST CANCER DETECTION KIT OR DEVICE, AND DETECTION METHOD

(71) Applicants: TORAY INDUSTRIES, INC., Tokyo (JP); NATIONAL CANCER CENTER, Tokyo (JP)

(72) Inventors: Satoshi KONDOU, Kamakura-shi (JP); Hitoshi NOBUMASA, Kamakura-shi (JP); Satoko KOZONO, Kamakura-shi (JP); Hiroko SUDO, Kamakura-shi (JP); Junpei KAWAUCHI, Kamakura-shi (JP); Takahiro OCHIYA, Tokyo (JP); Nobuyoshi KOSAKA, Tokyo (JP); Makiko ONO, Tokyo (JP); Kenji TAMURA, Tokyo (JP)

(73) Assignees: TORAY INDUSTRIES, INC., Tokyo (JP); NATIONAL CANCER CENTER, Tokyo (JP)

(21) Appl. No.: 18/514,603

(22) Filed: Nov. 20, 2023

Related U.S. Application Data
(62) Division of application No. 17/944,779, filed on Sep. 14, 2022, now Pat. No. 11,859,255.
Application No. 17/944,779 is a division of application No. 16/797,625, filed on Feb. 21, 2020, now Pat. No. 11,479,822.
Application No. 16/797,625 is a division of application No. 15/318,328, filed on Dec. 12, 2016, now Pat. No. 10,597,726.

(30) Foreign Application Priority Data
Jun. 13, 2014 (JP) 2014-122672
Mar. 30, 2015 (JP) 2015-069321

(51) Int. Cl. C12Q 1/6886; C12M 1/00; C12Q 1/68

(52) U.S. Cl. CPC C12Q 1/6886; C12M 1/00; C12Q 1/68; C12N 15/09

(57) ABSTRACT

It is intended to provide a kit or a device for the detection of breast cancer and a method for detecting breast cancer. The present invention provides a kit or a device for the detection of breast cancer, comprising nucleic acid(s) capable of specifically binding to a miRNA in a sample of a subject, and a method for detecting breast cancer, comprising measuring the miRNA in vitro.

### CROSS-REFERENCE TO RELATED APPLICATIONS

This application is a Divisional of U.S. application Ser. No. 17/944,779 filed Sep. 14, 2022, which is a Divisional of U.S. application Ser. No. 16/797,625 filed Feb. 21, 2020 (now U.S. Pat. No. 11,479,822), which is a Divisional of U.S. application Ser. No. 15/318,328, filed on Dec. 12, 2016 (now U.S. Pat. No. 10,597,726), which is the National Phase under 35 U.S.C. § 371 of International Application No. PCT/JP2015/066986 filed on Jun. 12, 2015, which claims the benefit under 35 U.S.C. § 119(a) to Japanese Patent Application Nos. 2014-122672 filed Jun. 13, 2014, and 2015-069321 filed Mar. 30, 2015, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 12, 2022, is named “PH-6234-PCT-US-DIV1-DIV1 Sequence Listing ST26” and is 784,115 bytes in size. The sequence listing contained in this .XML file is part of the specification and is hereby incorporated by reference herein in its entirety.

### TECHNICAL FIELD

The present invention relates to a kit or a device for the detection of breast cancer, comprising nucleic acid(s) capable of specifically binding to a particular miRNA, which is used for examining the presence or absence of breast cancer in a subject, and a method for detecting breast cancer, comprising measuring an expression level of the miRNA using the nucleic acid.

### BACKGROUND ART

The breast is constituted by a mammary gland which produces mother milk, lobules which arise from the mammary gland, mammary ducts which arise from the lobules and deliver milk, and fat which supports these constituents, etc. Approximately 90% of breast cancer cases originate in the mammary ducts, while approximately 5 to 10% of the breast cancer cases originate in the lobules (Non-Patent Literature 1). According to the 2011 statistics of cancer type-specific mortality in Japan disclosed by the Center for Cancer Control and Information Services, National Cancer Center, the number of breast cancer deaths climbed to 12,731 people. It is estimated that one out of 14 Japanese females will experience breast cancer. The number of incidences of this cancer in females takes the 1st place by cancer type. It is estimated that one out of 8 American females will experience breast cancer. The estimated number of American individuals affected by breast cancer climbed to 232,670 people in 2014, among which approximately 40,000 people reportedly died (Non-Patent Literature 1).

The stages of breast cancer progression are defined in Non-Patent Literature 2 and classified into stages 0, IA, IB, IIA, IIB, IIIA, IIIB, IIIC, and IV according to tumor size, infiltration, lymph node metastasis, distant metastasis, etc. The 5-year relative survival rate of breast cancer largely depends on the stages of cancer progression and is reportedly 100% for stage 0 and stage I, 93% for stage 11, 72% for stage 111, and 22% for stage IV (Non-Patent Literature 1). Thus, the early detection of breast cancer leads to improvement in the survival rate. Therefore, an approach that permits the early detection is strongly desired.

The treatment of breast cancer is basically surgical treatment, which is used in combination with drug therapy or radiotherapy depending on the progressed stage, metastasis, general health conditions, and breast cancer classification. Particularly, for early breast cancer of stage 1 or 2, breast conservation therapy may be selected with a combined use with radiotherapy (Non-Patent Literature 1).

According to Non-Patent Literature 1, initial diagnostic tests of breast cancer include inspection and palpation as well as imaging tests such as mammography, which is breast-dedicated X-ray examination, and ultrasonography (echo examination). When there are findings on suspected breast cancer by the initial test, pathological examination which involves inserting a needle into a lesion and collecting cells or tissues to be examined under a microscope, is carried out as a secondary test. If necessary, imaging tests such as CT, MRI, abdominal ultrasonography, bone scintigraphy, and PET are also carried out in order to examine the state or spread of the lesion.

For example, CEA, CA-15-3, and CA27-29 are known as tumor markers for the detection of breast cancer. These tumor markers in blood have been reported to elevate when breast cancer has metastasized to other organs such as the bone or the liver. However, these tumor markers do not elevate in some patients and may thus be limited by their usefulness (Non-Patent Literature 1).

As shown in Patent Literatures 1 to 4, there are reports, albeit at a research stage, on the detection of breast cancer using the expression levels of microRNAs (miRNAs) or combinations of the expression levels of miRNAs and the expression levels of additional protein markers in biological samples including blood.

Specifically, Patent Literature 1 discloses a method for detecting prostate cancer or other cancers including breast cancer by combining hsa-miR-602 or hsa-miR-135a-3p with known protein markers in blood.

Patent Literature 2 discloses a method for detecting various cancers including breast cancer by combining hsa-miR-23b-3p or hsa-miR-135a-3p with 5 or more other miRNAs in blood or tissues.

Patent Literature 3 discloses a method for detecting breast cancer using hsa-miR-92a-3p, hsa-miR-92a-2-5p, hsa-miR-92b-5p, and the like in blood cells.

Patent Literature 4 discloses a method for detecting breast cancer using hsa-miR-451a, hsa-miR-296-5p, hsa-miR-16-5p, and the like in tissues.

Non-Patent Literature 3 discloses that hsa-miR-760 and the like in blood are significantly expressed in breast cancer patients.

Non-Patent Literature 4 discloses that hsa-miR-423-5p, hsa-miR-486-5p, and the like in blood are decreased after surgery of breast cancer.

Non-Patent Literature 5 discloses that hsa-miR-4257, hsa-miR-1915-3p, hsa-miR-718, and the like in blood are significantly expressed in breast cancer patients.

Non-Patent Literature 6 discloses that hsa-miR-940 and the like in blood are significantly expressed in breast cancer patients.

### CITATION LIST

### Patent Literature

Patent Literature 1: JP Patent Publication (Kohyo) No. 2012-507300 A (2012)
Patent Literature 2: JP Patent Publication (Kohyo) No. 2008-500837 A (2008)
Patent Literature 3: International Publication No. WO 10/123043
Patent Literature 4: Published U.S. Patent Application No. 2008/0076674

### Non-Patent Literature

Non-Patent Literature 1: American Cancer Society, “Breast Cancer”, 2013, p. 6-9, 13, 27-28, 41-46, 52-54, 63-64, and 106
Non-Patent Literature 2: Sobin, L. et al., “TNM Classification of Malignant Tumours, the 7th edition”, 2010, p. 171 to 181
Non-Patent Literature 3: Godfrey, A C. et al., 2013, Breast Cancer Research, Vol. 15 (3), p. R42
Non-Patent Literature 4: Cookson, V J. et al., 2012, Cellular Oncology, Vol. 35 (4), p. 301-8
Non-Patent Literature 5: Schrauder, M G. et al., 2012, PLoS One, Vol. 7 (1), p. e29770
Non-Patent Literature 6: Leidner, R S. et al., 2013, PLoS One, Vol. 8 (3), p. e57841
Non-Patent Literature 7: Tamaki, K. et al., 2013, Japanese Journal of Clinical Oncology, Vol. 43 (2), p. 208-213
Non-Patent Literature 8: Guadagni, F. et al., 2001, Clinical Cancer Research, Vol. 7, p. 2357 to 2362

### SUMMARY OF INVENTION

### Problem to be Solved by Invention

An object of the present invention is to find novel tumor markers for breast cancer and to provide a method that can effectively detect breast cancer using nucleic acid(s) capable of specifically binding to the markers. As described in Non-Patent Literature 1, initial diagnostic tests of breast cancer include inspection and palpation as well as imaging tests such as mammography, which is breast-dedicated X-ray examination, and ultrasonography. The mammography is reportedly effective as breast cancer examination targeting women aged 40 or older, and the American Cancer Society recommends that women in this age range take mammography every year (Non-Patent Literature 1). The mammography, however, has been reported to have limitations in the visualization of breast cancer present in the dense breast before menopause or a very small tumor of early breast cancer (Non-Patent Literature 1). In Japan, the mammography rate was only 24.3% in 2010, and a challenge to improvement in breast cancer survival rate will be to increase this mammography rate (Non-Patent Literature 7).

For example, CEA, CA-15-3, and CA27-29 mentioned above are known as tumor markers for the detection of breast cancer. These tumor markers, however, are helpful in confirming therapeutic effects on recurrent breast cancer, but rarely elevate in early breast cancer. Therefore, the tumor markers may not be useful for the purpose of breast cancer examination (Non-Patent Literature 1). According to Non-Patent Literature 8, the specific sensitivity of CEA and CA15-3 is uselessly 6.4% and 12.2%, respectively, for stage 1 and is only 25.0% and 62.5%, respectively, even for stage 4. Thus, the tumor marker measurement is less significant as a preoperative test. Since these blood tumor markers may elevate for reasons other than breast cancer, the presence or absence of breast cancer is difficult to determine. The false diagnosis of other cancers as breast cancer wastes appropriate therapeutic opportunity or places unnecessary economical and physical burdens on patients due to the application of wrong medicine.

As described below, there are reports, albeit at a research stage, on the determination of breast cancer using the expression levels of microRNAs (miRNAs) in biological samples including blood, none of which, however, have yet been brought into practical use.

Patent Literature 1 discloses a method for detecting prostate cancer or other cancers including breast cancer by combining hsa-miR-602 or hsa-miR-135a-3p with known protein markers in blood. The measurement of both miRNA and protein markers, however, brings about increase in examination costs and a complicated process and is therefore not favorable. This detection method does not describe specific detection performance such as accuracy, sensitivity, or specificity for determining breast cancer and is thus industrially less practical.

Patent Literature 2 discloses a method for detecting various cancers including breast cancer by combining hsa-miR-23b-3p or hsa-miR-135a-3p with 5 or more other miRNAs in blood or tissues. This detection method does not describe specific detection performance such as accuracy, sensitivity, or specificity for determining breast cancer and is thus industrially less practical.

Patent Literature 3 describes a method for detecting breast cancer using hsa-miR-92a-3p, hsa-miR-92a-2-5p, hsa-miR-92b-5p, and the like. This detection method does not describe specific detection performance such as accuracy, sensitivity, or specificity for determining breast cancer and is thus industrially less practical. In addition, these miRNA markers were not validated in an independent sample group and are therefore less reliable.

Patent Literature 4 discloses a method for detecting breast cancer using hsa-miR-451a, hsa-miR-296-5p, hsa-miR-16-5p, and the like in tissues. For this detection method, however, tissue resection by surgical operation is essential for obtaining samples, and this step places a heavy physical burden on patients. Therefore, this method is not favorable as an examination method. In addition, this detection method does not describe specific detection performance such as accuracy, sensitivity, or specificity for determining breast cancer and is thus industrially less practical.

Non-Patent Literature 3 discloses that hsa-miR-760 and the like in blood are significantly expressed in breast cancer patients. This literature, however, neither describes detection performance such as accuracy, sensitivity, or specificity for determining breast cancer nor describes a specific method for detecting breast cancer. Therefore, this approach is industrially less practical.

Non-Patent Literature 4 discloses that hsa-miR-423-5p, hsa-miR-486-5p, and the like in blood are decreased after surgery of breast cancer. This literature, however, neither describes detection performance such as accuracy, sensitivity, or specificity for determining breast cancer nor describes a specific method for detecting breast cancer. Therefore, this approach is industrially less practical.

Non-Patent Literature 5 discloses that hsa-miR-4257, hsa-miR-1915-3p, hsa-miR-718, and the like in blood are significantly expressed in breast cancer patients. This approach, however, employed as many as 240 miRNAs for detecting breast cancer and might cause increase in examination cost and complicated discriminant algorithms. Thus, this approach is not industrially practical.

Non-Patent Literature 6 discloses that hsa-miR-940 and the like in blood are significantly expressed in breast cancer patients. The authors, however, concluded that this marker is less reproducible, and finally abandoned the marker in the study. In addition, this literature neither describes detection performance such as accuracy, sensitivity, or specificity for determining breast cancer nor describes a specific method for detecting breast cancer. Therefore, this approach is industrially less practical.

As mentioned above, the existing tumor markers exhibit low performance in the detection of breast cancer, or neither performance nor detection methods are specifically shown as to the markers at a research stage. Therefore, use of these markers might impose implementation of needless extra examination due to the false detection of healthy subjects as being breast cancer patients, or might waste therapeutic opportunity because of overlooking breast cancer patients. In addition, the measurement of dozens of miRNAs increases examination cost and is therefore difficult to use in large-scale screening such as medical checkup. Furthermore, the collection of breast tissues for measuring the tumor markers is highly invasive to patients and is not favorable. Hence, there is a demand for a highly accurate breast cancer marker that is detectable from blood, which can be collected with limitedly invasiveness, and is capable of correctly determining a breast cancer patient as a breast cancer patient and a healthy subject as a healthy subject. Particularly, the early detection and treatment of breast cancer can drastically reduce the risk of recurrence and also permit breast conservation therapy. Therefore, a highly sensitive breast cancer marker capable of detecting breast cancer even at a low progressed stage is desired. Moreover, the mammography rate is presumably increased by providing a more convenient initial screening of breast cancer.

### Solution to Problem

The present inventors have conducted diligent studies to attain the object and consequently completed the present invention by finding several genes usable as markers for the detection of breast cancer from blood, which can be collected with limitedly invasiveness, and finding that breast cancer can be significantly detected by using nucleic acids capable of specifically binding to any of these markers.

### SUMMARY OF INVENTION

The present invention has the following features:

(1) A kit for the detection of breast cancer, comprising nucleic acid(s) capable of specifically binding to at least one polynucleotide selected from the group consisting of the following breast cancer markers: miR-4783-3p, miR-4730, miR-1307-3p, miR-4634, miR-663a, miR-4532, miR-7704, miR-3178, miR-6729-5p, miR-6090, miR-4732-5p, miR-3184-5p, miR-6727-5p, miR-6088, miR-4674, miR-8073, miR-4787-5p, miR-1469, miR-125a-3p, miR-1233-5p, miR-885-3p, miR-6802-5p, miR-328-5p, miR-6787-5p, miR-8069, miR-6875-5p, miR-1246, miR-4734, miR-6757-5p, miR-6756-5p, miR-3665, miR-6836-3p, miR-6821-5p, miR-6805-5p, miR-4728-5p, miR-6726-5p, miR-197-5p, miR-149-3p, miR-6850-5p, miR-4476, miR-6858-5p, miR-564, miR-4763-3p, miR-575, miR-6771-5p, miR-1231, miR-1908-3p, miR-150-3p, miR-3937, miR-887-3p, miR-3940-5p, miR-4741, miR-6808-5p, miR-6869-5p, miR-5090, miR-615-5p, miR-8072, miR-128-1-5p, miR-1238-5p, miR-365a-5p, miR-204-3p, miR-4492, miR-6785-5p, miR-6511a-5p, miR-4525, miR-1915-5p, miR-3180, miR-6879-5p, miR-1199-5p, miR-6746-5p, miR-711, miR-663b, miR-4707-3p, miR-6893-5p, miR-4675, miR-4638-5p, miR-4651, miR-6087, miR-4665-5p, miR-4758-5p, miR-6887-5p, miR-3620-5p, miR-1909-3p, miR-7641, miR-6724-5p, miR-1343-3p, miR-6780b-5p, miR-4484, miR-4690-5p, miR-4429, miR-1227-5p, miR-4725-3p, miR-6861-5p, miR-6812-5p, miR-3197, miR-8059, miR-3185, miR-4706, miR-4497, miR-3131, miR-6806-5p, miR-187-5p, miR-3180-3p, miR-6848-5p, miR-6820-5p, miR-6800-5p, miR-6717-5p, miR-6795-5p, miR-4632-5p, miR-665, miR-6778-5p, miR-3663-3p, miR-4689, miR-211-3p, miR-6511b-5p, miR-4750-5p, miR-6126, miR-614, miR-7110-5p, miR-744-5p, miR-6769a-5p, miR-4792, miR-5787, miR-6798-5p, miR-6781-5p, miR-4419b, miR-4446-3p, miR-4259, miR-5572, miR-6075, miR-296-3p, miR-6891-5p, miR-4745-5p, miR-6775-5p, miR-6870-5p, miR-920, miR-4530, miR-6819-5p, miR-6825-5p, miR-7847-3p, miR-6131, miR-4433-3p, miR-1228-5p, miR-6743-5p, miR-1268a, miR-3917, miR-6786-5p, miR-3154, miR-638, miR-6741-5p, miR-6889-5p, miR-6840-3p, miR-6510-5p, miR-3188, miR-551b-5p, miR-5001-5p, miR-1268b, miR-7107-5p, miR-6824-5p, miR-6732-5p, miR-371a-5p, miR-6794-5p, miR-6779-5p, miR-4271, miR-5195-3p, miR-6762-5p, miR-939-5p, miR-1247-3p, miR-6777-5p, miR-6722-3p, miR-3656, miR-4688, miR-3195, miR-6766-5p, miR-4447, miR-4656, miR-7108-5p, miR-3191-3p, miR-1273g-3p, miR-4463, miR-2861, miR-3196, miR-6877-5p, miR-3679-5p, miR-4442, miR-6789-5p, miR-6782-5p, miR-486-3p, miR-6085, miR-4746-3p, miR-619-5p, miR-937-5p, miR-6803-5p, miR-4298, miR-4454, miR-4459, miR-7150, miR-6880-5p, miR-4449, miR-8063, miR-4695-5p, miR-6132, miR-6829-5p, miR-4486, miR-6805-3p, miR-6826-5p, miR-4508, miR-1343-5p, miR-7114-5p, miR-3622a-5p, miR-6765-5p, miR-7845-5p, miR-3960, miR-6749-5p, miR-1260b, miR-6799-5p, miR-4723-5p, miR-6784-5p, miR-5100, miR-6769b-5p, miR-1207-5p, miR-642a-3p, miR-4505, miR-4270, miR-6721-5p, miR-7111-5p, miR-6791-5p, miR-7109-5p, miR-4258, miR-6515-3p, miR-6851-5p, miR-6125, miR-4749-5p, miR-4726-5p, miR-4513, miR-6089, miR-6816-5p, miR-4466, miR-4488, miR-6752-5p and miR-4739.

(2) The kit according to (1), wherein miR-4783-3p is hsa-miR-4783-3p, miR-4730 is hsa-miR-4730, miR-1307-3p is hsa-miR-1307-3p, miR-4634 is hsa-miR-4634, miR-663a is hsa-miR-663a, miR-4532 is hsa-miR-4532, miR-7704 is hsa-miR-7704, miR-3178 is hsa-miR-3178, miR-6729-5p is hsa-miR-6729-5p, miR-6090 is hsa-miR-6090, miR-4732-5p is hsa-miR-4732-5p, miR-3184-5p is hsa-miR-3184-5p, miR-6727-5p is hsa-miR-6727-5p, miR-6088 is hsa-miR-6088, miR-4674 is hsa-miR-4674, miR-8073 is hsa-miR-8073, miR-4787-5p is hsa-miR-4787-5p, miR-1469 is hsa-miR-1469, miR-125a-3p is hsa-miR-125a-3p, miR-1233-5p is hsa-miR-1233-5p, miR-885-3p is hsa-miR-885-3p, miR-6802-5p is hsa-miR-6802-5p, miR-328-5p is hsa-miR-328-5p, miR-6787-5p is hsa-miR-6787-5p, miR-8069 is hsa-miR-8069, miR-6875-5p is hsa-miR-6875-5p, miR-1246 is hsa-miR-1246, miR-4734 is hsa-miR-4734, miR-6757-5p is hsa-miR-6757-5p, miR-6756-5p is hsa-miR-6756-5p, miR-3665 is hsa-miR-3665, miR-6836-3p is hsa-miR-6836-3p, miR-6821-5p is hsa-miR-6821-5p, miR-6805-5p is hsa-miR-6805-5p, miR-4728-5p is hsa-miR-4728-5p, miR-6726-5p is hsa-miR-6726-5p, miR-197-5p is hsa-miR-197-5p, miR-149-3p is hsa-miR-149-3p, miR-6850-5p is hsa-miR-6850-5p, miR-4476 is hsa-miR-4476, miR-6858-5p is hsa-miR-6858-5p, miR-564 is hsa-miR-564, miR-4763-3p is hsa-miR-4763-3p, miR-575 is hsa-miR-575, miR-6771-5p is hsa-miR-6771-5p, miR-1231 is hsa-miR-1231, miR-1908-3p is hsa-miR-1908-3p, miR-150-3p is hsa-miR-150-3p, miR-3937 is hsa-miR-3937, miR-887-3p is hsa-miR-887-3p, miR-3940-5p is hsa-miR-3940-5p, miR-4741 is hsa-miR-4741, miR-6808-5p is hsa-miR-6808-5p, miR-6869-5p is hsa-miR-6869-5p, miR-5090 is hsa-miR-5090, miR-615-5p is hsa-miR-615-5p, miR-8072 is hsa-miR-8072, miR-128-1-5p is hsa-miR-128-1-5p, miR-1238-5p is hsa-miR-1238-5p, miR-365a-5p is hsa-miR-365a-5p, miR-204-3p is hsa-miR-204-3p, miR-4492 is hsa-miR-4492, miR-6785-5p is hsa-miR-6785-5p, miR-6511a-5p is hsa-miR-6511a-5p, miR-4525 is hsa-miR-4525, miR-1915-5p is hsa-miR-1915-5p, miR-3180 is hsa-miR-3180, miR-6879-5p is hsa-miR-6879-5p, miR-1199-5p is hsa-miR-1199-5p, miR-6746-5p is hsa-miR-6746-5p, miR-711 is hsa-miR-711, miR-663b is hsa-miR-663b, miR-4707-3p is hsa-miR-4707-3p, miR-6893-5p is hsa-miR-6893-5p, miR-4675 is hsa-miR-4675, miR-4638-5p is hsa-miR-4638-5p, miR-4651 is hsa-miR-4651, miR-6087 is hsa-miR-6087, miR-4665-5p is hsa-miR-4665-5p, miR-4758-5p is hsa-miR-4758-5p, miR-6887-5p is hsa-miR-6887-5p, miR-3620-5p is hsa-miR-3620-5p, miR-1909-3p is hsa-miR-1909-3p, miR-7641 is hsa-miR-7641, miR-6724-5p is hsa-miR-6724-5p, miR-1343-3p is hsa-miR-1343-3p, miR-6780b-5p is hsa-miR-6780b-5p, miR-4484 is hsa-miR-4484, miR-4690-5p is hsa-miR-4690-5p, miR-4429 is hsa-miR-4429, miR-1227-5p is hsa-miR-1227-5p, miR-4725-3p is hsa-miR-4725-3p, miR-6861-5p is hsa-miR-6861-5p, miR-6812-5p is hsa-miR-6812-5p, miR-3197 is hsa-miR-3197, miR-8059 is hsa-miR-8059, miR-3185 is hsa-miR-3185, miR-4706 is hsa-miR-4706, miR-4497 is hsa-miR-4497, miR-3131 is hsa-miR-3131, miR-6806-5p is hsa-miR-6806-5p, miR-187-5p is hsa-miR-187-5p, miR-3180-3p is hsa-miR-3180-3p, miR-6848-5p is hsa-miR-6848-5p, miR-6820-5p is hsa-miR-6820-5p, miR-6800-5p is hsa-miR-6800-5p, miR-6717-5p is hsa-miR-6717-5p, miR-6795-5p is hsa-miR-6795-5p, miR-4632-5p is hsa-miR-4632-5p, miR-665 is hsa-miR-665, miR-6778-5p is hsa-miR-6778-5p, miR-3663-3p is hsa-miR-3663-3p, miR-4689 is hsa-miR-4689, miR-211-3p is hsa-miR-211-3p, miR-6511b-5p is hsa-miR-6511b-5p, miR-4750-5p is hsa-miR-4750-5p, miR-6126 is hsa-miR-6126, miR-614 is hsa-miR-614, miR-7110-5p is hsa-miR-7110-5p, miR-744-5p is hsa-miR-744-5p, miR-6769a-5p is hsa-miR-6769a-5p, miR-4792 is hsa-miR-4792, miR-5787 is hsa-miR-5787, miR-6798-5p is hsa-miR-6798-5p, miR-6781-5p is hsa-miR-6781-5p, miR-4419b is hsa-miR-4419b, miR-4446-3p is hsa-miR-4446-3p, miR-4259 is hsa-miR-4259, miR-5572 is hsa-miR-5572, miR-6075 is hsa-miR-6075, miR-296-3p is hsa-miR-296-3p, miR-6891-5p is hsa-miR-6891-5p, miR-4745-5p is hsa-miR-4745-5p, miR-6775-5p is hsa-miR-6775-5p, miR-6870-5p is hsa-miR-6870-5p, miR-920 is hsa-miR-920, miR-4530 is hsa-miR-4530, miR-6819-5p is hsa-miR-6819-5p, miR-6825-5p is hsa-miR-6825-5p, miR-7847-3p is hsa-miR-7847-3p, miR-6131 is hsa-miR-6131, miR-4433-3p is hsa-miR-4433-3p, miR-1228-5p is hsa-miR-1228-5p, miR-6743-5p is hsa-miR-6743-5p, miR-1268a is hsa-miR-1268a, miR-3917 is hsa-miR-3917, miR-6786-5p is hsa-miR-6786-5p, miR-3154 is hsa-miR-3154, miR-638 is hsa-miR-638, miR-6741-5p is hsa-miR-6741-5p, miR-6889-5p is hsa-miR-6889-5p, miR-6840-3p is hsa-miR-6840-3p, miR-6510-5p is hsa-miR-6510-5p, miR-3188 is hsa-miR-3188, miR-551b-5p is hsa-miR-551b-5p, miR-5001-5p is hsa-miR-5001-5p, miR-1268b is hsa-miR-1268b, miR-7107-5p is hsa-miR-7107-5p, miR-6824-5p is hsa-miR-6824-5p, miR-6732-5p is hsa-miR-6732-5p, miR-371a-5p is hsa-miR-371a-5p, miR-6794-5p is hsa-miR-6794-5p, miR-6779-5p is hsa-miR-6779-5p, miR-4271 is hsa-miR-4271, miR-5195-3p is hsa-miR-5195-3p, miR-6762-5p is hsa-miR-6762-5p, miR-939-5p is hsa-miR-939-5p, miR-1247-3p is hsa-miR-1247-3p, miR-6777-5p is hsa-miR-6777-5p, miR-6722-3p is hsa-miR-6722-3p, miR-3656 is hsa-miR-3656, miR-4688 is hsa-miR-4688, miR-3195 is hsa-miR-3195, miR-6766-5p is hsa-miR-6766-5p, miR-4447 is hsa-miR-4447, miR-4656 is hsa-miR-4656, miR-7108-5p is hsa-miR-7108-5p, miR-3191-3p is hsa-miR-3191-3p, miR-1273g-3p is hsa-miR-1273g-3p, miR-4463 is hsa-miR-4463, miR-2861 is hsa-miR-2861, miR-3196 is hsa-miR-3196, miR-6877-5p is hsa-miR-6877-5p, miR-3679-5p is hsa-miR-3679-5p, miR-4442 is hsa-miR-4442, miR-6789-5p is hsa-miR-6789-5p, miR-6782-5p is hsa-miR-6782-5p, miR-486-3p is hsa-miR-486-3p, miR-6085 is hsa-miR-6085, miR-4746-3p is hsa-miR-4746-3p, miR-619-5p is hsa-miR-619-5p, miR-937-5p is hsa-miR-937-5p, miR-6803-5p is hsa-miR-6803-5p, miR-4298 is hsa-miR-4298, miR-4454 is hsa-miR-4454, miR-4459 is hsa-miR-4459, miR-7150 is hsa-miR-7150, miR-6880-5p is hsa-miR-6880-5p, miR-4449 is hsa-miR-4449, miR-8063 is hsa-miR-8063, miR-4695-5p is hsa-miR-4695-5p, miR-6132 is hsa-miR-6132, miR-6829-5p is hsa-miR-6829-5p, miR-4486 is hsa-miR-4486, miR-6805-3p is hsa-miR-6805-3p, miR-6826-5p is hsa-miR-6826-5p, miR-4508 is hsa-miR-4508, miR-1343-5p is hsa-miR-1343-5p, miR-7114-5p is hsa-miR-7114-5p, miR-3622a-5p is hsa-miR-3622a-5p, miR-6765-5p is hsa-miR-6765-5p, miR-7845-5p is hsa-miR-7845-5p, miR-3960 is hsa-miR-3960, miR-6749-5p is hsa-miR-6749-5p, miR-1260b is hsa-miR-1260b, miR-6799-5p is hsa-miR-6799-5p, miR-4723-5p is hsa-miR-4723-5p, miR-6784-5p is hsa-miR-6784-5p, miR-5100 is hsa-miR-5100, miR-6769b-5p is hsa-miR-6769b-5p, miR-1207-5p is hsa-miR-1207-5p, miR-642a-3p is hsa-miR-642a-3p, miR-4505 is hsa-miR-4505, miR-4270 is hsa-miR-4270, miR-6721-5p is hsa-miR-6721-5p, miR-7111-5p is hsa-miR-7111-5p, miR-6791-5p is hsa-miR-6791-5p, miR-7109-5p is hsa-miR-7109-5p, miR-4258 is hsa-miR-4258, miR-6515-3p is hsa-miR-6515-3p, miR-6851-5p is hsa-miR-6851-5p, miR-6125 is hsa-miR-6125, miR-4749-5p is hsa-miR-4749-5p, miR-4726-5p is hsa-miR-4726-5p, miR-4513 is hsa-miR-4513, miR-6089 is hsa-miR-6089, miR-6816-5p is hsa-miR-6816-5p, miR-4466 is hsa-miR-4466, miR-4488 is hsa-miR-4488, miR-6752-5p is hsa-miR-6752-5p, and miR-4739 is hsa-miR-4739.

(3) The kit according to (1) or (2), wherein the nucleic acid is a polynucleotide selected from the group consisting of the following polynucleotides (a) to (e):
(a) a polynucleotide consisting of a nucleotide sequence represented by any of SEQ ID NOs: 1 to 235 and 851 to 856 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 235 and 851 to 856 or a nucleotide sequence derived from the nucleotide sequence by the replacement of u with t,
(c) a polynucleotide consisting of a nucleotide sequence complementary to a nucleotide sequence represented by any of SEQ ID NOs: 1 to 235 and 851 to 856 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 235 and 851 to 856 or a nucleotide sequence derived from the nucleotide sequence by the replacement of u with t, and
(e) a polynucleotide hybridizing under stringent conditions to any of the polynucleotides (a) to (d).

(4) The kit according to any of (1) to (3), wherein the kit further comprises nucleic acid(s) capable of specifically binding to at least one polynucleotide selected from the group consisting of other breast cancer markers miR-760, miR-602, miR-423-5p, miR-92a-2-5p, miR-16-5p, miR-451a, miR-135a-3p, miR-486-5p, miR-4257, miR-92b-5p, miR-1915-3p, miR-718, miR-940, miR-296-5p, miR-23b-3p and miR-92a-3p.

(5) The kit according to (4), wherein miR-760 is hsa-miR-760, miR-602 is hsa-miR-602, miR-423-5p is hsa-miR-423-5p, miR-92a-2-5p is hsa-miR-92a-2-5p, miR-16-5p is hsa-miR-16-5p, miR-451a is hsa-miR-451a, miR-135a-3p is hsa-miR-135a-3p, miR-486-5p is hsa-miR-486-5p, miR-4257 is hsa-miR-4257, miR-92b-5p is hsa-miR-92b-5p, miR-1915-3p is hsa-miR-1915-3p, miR-718 is hsa-miR-718, miR-940 is hsa-miR-940, miR-296-5p is hsa-miR-296-5p, miR-23b-3p is hsa-miR-23b-3p, and miR-92a-3p is hsa-miR-92a-3p.

(6) The kit according to (4) or (5), wherein the nucleic acid is a polynucleotide selected from the group consisting of the following polynucleotides (f) to (j):
(f) a polynucleotide consisting of a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 236 to 251,
(h) a polynucleotide consisting of a nucleotide sequence complementary to a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 236 to 251 or a nucleotide sequence derived from the nucleotide sequence by the replacement of u with t, and
(j) a polynucleotide hybridizing under stringent conditions to any of the polynucleotides (f) to (i).

(7) The kit according to any of (1) to (6), wherein the kit further comprises nucleic acid(s) capable of specifically binding to at least one polynucleotide selected from the group consisting of other breast cancer markers miR-658, miR-6842-5p, miR-6124, miR-6765-3p, miR-7106-5p, miR-4534, miR-92b-3p, miR-3135b, miR-4687-3p, miR-762, miR-3619-3p, miR-4467, miR-557, miR-1237-5p, miR-1908-5p, miR-4286, miR-6885-5p and miR-6763-5p.

(8) The kit according to (7), wherein miR-658 is hsa-miR-658, miR-6842-5p is hsa-miR-6842-5p, miR-6124 is hsa-miR-6124, miR-6765-3p is hsa-miR-6765-3p, miR-7106-5p is hsa-miR-7106-5p, miR-4534 is hsa-miR-4534, miR-92b-3p is hsa-miR-92b-3p, miR-3135b is hsa-miR-3135b, miR-4687-3p is hsa-miR-4687-3p, miR-762 is hsa-miR-762, miR-3619-3p is hsa-miR-3619-3p, miR-4467 is hsa-miR-4467, miR-557 is hsa-miR-557, miR-1237-5p is hsa-miR-1237-5p, miR-1908-5p is hsa-miR-1908-5p, miR-4286 is hsa-miR-4286, miR-6885-5p is hsa-miR-6885-5p, and miR-6763-5p is hsa-miR-6763-5p.

(9) The kit according to (7) or (8), wherein the nucleic acid is a polynucleotide selected from the group consisting of the following polynucleotides (k) to (o):
(k) a polynucleotide consisting of a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 252 to 269,
(m) a polynucleotide consisting of a nucleotide sequence complementary to a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 252 to 269 or a nucleotide sequence derived from the nucleotide sequence by the replacement of u with t, and
(o) a polynucleotide hybridizing under stringent conditions to any of the polynucleotides (k) to (n).

(10) The kit according to any of (1) to (9), wherein the kit comprises at least two nucleic acids capable of specifically binding to at least two polynucleotides, respectively, selected from the group consisting of all of the breast cancer markers according to (1) or (2).

(11) A device for the detection of breast cancer, comprising nucleic acid(s) capable of specifically binding to at least one polynucleotide selected from the group consisting of the following breast cancer markers: miR-4783-3p, miR-4730, miR-1307-3p, miR-4634, miR-663a, miR-4532, miR-7704, miR-3178, miR-6729-5p, miR-6090, miR-4732-5p, miR-3184-5p, miR-6727-5p, miR-6088, miR-4674, miR-8073, miR-4787-5p, miR-1469, miR-125a-3p, miR-1233-5p, miR-885-3p, miR-6802-5p, miR-328-5p, miR-6787-5p, miR-8069, miR-6875-5p, miR-1246, miR-4734, miR-6757-5p, miR-6756-5p, miR-3665, miR-6836-3p, miR-6821-5p, miR-6805-5p, miR-4728-5p, miR-6726-5p, miR-197-5p, miR-149-3p, miR-6850-5p, miR-4476, miR-6858-5p, miR-564, miR-4763-3p, miR-575, miR-6771-5p, miR-1231, miR-1908-3p, miR-150-3p, miR-3937, miR-887-3p, miR-3940-5p, miR-4741, miR-6808-5p, miR-6869-5p, miR-5090, miR-615-5p, miR-8072, miR-128-1-5p, miR-1238-5p, miR-365a-5p, miR-204-3p, miR-4492, miR-6785-5p, miR-6511a-5p, miR-4525, miR-1915-5p, miR-3180, miR-6879-5p, miR-1199-5p, miR-6746-5p, miR-711, miR-663b, miR-4707-3p, miR-6893-5p, miR-4675, miR-4638-5p, miR-4651, miR-6087, miR-4665-5p, miR-4758-5p, miR-6887-5p, miR-3620-5p, miR-1909-3p, miR-7641, miR-6724-5p, miR-1343-3p, miR-6780b-5p, miR-4484, miR-4690-5p, miR-4429, miR-1227-5p, miR-4725-3p, miR-6861-5p, miR-6812-5p, miR-3197, miR-8059, miR-3185, miR-4706, miR-4497, miR-3131, miR-6806-5p, miR-187-5p, miR-3180-3p, miR-6848-5p, miR-6820-5p, miR-6800-5p, miR-6717-5p, miR-6795-5p, miR-4632-5p, miR-665, miR-6778-5p, miR-3663-3p, miR-4689, miR-211-3p, miR-6511b-5p, miR-4750-5p, miR-6126, miR-614, miR-7110-5p, miR-744-5p, miR-6769a-5p, miR-4792, miR-5787, miR-6798-5p, miR-6781-5p, miR-4419b, miR-4446-3p, miR-4259, miR-5572, miR-6075, miR-296-3p, miR-6891-5p, miR-4745-5p, miR-6775-5p, miR-6870-5p, miR-920, miR-4530, miR-6819-5p, miR-6825-5p, miR-7847-3p, miR-6131, miR-4433-3p, miR-1228-5p, miR-6743-5p, miR-1268a, miR-3917, miR-6786-5p, miR-3154, miR-638, miR-6741-5p, miR-6889-5p, miR-6840-3p, miR-6510-5p, miR-3188, miR-551b-5p, miR-5001-5p, miR-1268b, miR-7107-5p, miR-6824-5p, miR-6732-5p, miR-371a-5p, miR-6794-5p, miR-6779-5p, miR-4271, miR-5195-3p, miR-6762-5p, miR-939-5p, miR-1247-3p, miR-6777-5p, miR-6722-3p, miR-3656, miR-4688, miR-3195, miR-6766-5p, miR-4447, miR-4656, miR-7108-5p, miR-3191-3p, miR-1273g-3p, miR-4463, miR-2861, miR-3196, miR-6877-5p, miR-3679-5p, miR-4442, miR-6789-5p, miR-6782-5p, miR-486-3p, miR-6085, miR-4746-3p, miR-619-5p, miR-937-5p, miR-6803-5p, miR-4298, miR-4454, miR-4459, miR-7150, miR-6880-5p, miR-4449, miR-8063, miR-4695-5p, miR-6132, miR-6829-5p, miR-4486, miR-6805-3p, miR-6826-5p, miR-4508, miR-1343-5p, miR-7114-5p, miR-3622a-5p, miR-6765-5p, miR-7845-5p, miR-3960, miR-6749-5p, miR-1260b, miR-6799-5p, miR-4723-5p, miR-6784-5p, miR-5100, miR-6769b-5p, miR-1207-5p, miR-642a-3p, miR-4505, miR-4270, miR-6721-5p, miR-7111-5p, miR-6791-5p, miR-7109-5p, miR-4258, miR-6515-3p, miR-6851-5p, miR-6125, miR-4749-5p, miR-4726-5p, miR-4513, miR-6089, miR-6816-5p, miR-4466, miR-4488, miR-6752-5p and miR-4739.

(12) The device according to (11), wherein miR-4783-3p is hsa-miR-4783-3p, miR-4730 is hsa-miR-4730, miR-1307-3p is hsa-miR-1307-3p, miR-4634 is hsa-miR-4634, miR-663a is hsa-miR-663a, miR-4532 is hsa-miR-4532, miR-7704 is hsa-miR-7704, miR-3178 is hsa-miR-3178, miR-6729-5p is hsa-miR-6729-5p, miR-6090 is hsa-miR-6090, miR-4732-5p is hsa-miR-4732-5p, miR-3184-5p is hsa-miR-3184-5p, miR-6727-5p is hsa-miR-6727-5p, miR-6088 is hsa-miR-6088, miR-4674 is hsa-miR-4674, miR-8073 is hsa-miR-8073, miR-4787-5p is hsa-miR-4787-5p, miR-1469 is hsa-miR-1469, miR-125a-3p is hsa-miR-125a-3p, miR-1233-5p is hsa-miR-1233-5p, miR-885-3p is hsa-miR-885-3p, miR-6802-5p is hsa-miR-6802-5p, miR-328-5p is hsa-miR-328-5p, miR-6787-5p is hsa-miR-6787-5p, miR-8069 is hsa-miR-8069, miR-6875-5p is hsa-miR-6875-5p, miR-1246 is hsa-miR-1246, miR-4734 is hsa-miR-4734, miR-6757-5p is hsa-miR-6757-5p, miR-6756-5p is hsa-miR-6756-5p, miR-3665 is hsa-miR-3665, miR-6836-3p is hsa-miR-6836-3p, miR-6821-5p is hsa-miR-6821-5p, miR-6805-5p is hsa-miR-6805-5p, miR-4728-5p is hsa-miR-4728-5p, miR-6726-5p is hsa-miR-6726-5p, miR-197-5p is hsa-miR-197-5p, miR-149-3p is hsa-miR-149-3p, miR-6850-5p is hsa-miR-6850-5p, miR-4476 is hsa-miR-4476, miR-6858-5p is hsa-miR-6858-5p, miR-564 is hsa-miR-564, miR-4763-3p is hsa-miR-4763-3p, miR-575 is hsa-miR-575, miR-6771-5p is hsa-miR-6771-5p, miR-1231 is hsa-miR-1231, miR-1908-3p is hsa-miR-1908-3p, miR-150-3p is hsa-miR-150-3p, miR-3937 is hsa-miR-3937, miR-887-3p is hsa-miR-887-3p, miR-3940-5p is hsa-miR-3940-5p, miR-4741 is hsa-miR-4741, miR-6808-5p is hsa-miR-6808-5p, miR-6869-5p is hsa-miR-6869-5p, miR-5090 is hsa-miR-5090, miR-615-5p is hsa-miR-615-5p, miR-8072 is hsa-miR-8072, miR-128-1-5p is hsa-miR-128-1-5p, miR-1238-5p is hsa-miR-1238-5p, miR-365a-5p is hsa-miR-365a-5p, miR-204-3p is hsa-miR-204-3p, miR-4492 is hsa-miR-4492, miR-6785-5p is hsa-miR-6785-5p, miR-6511a-5p is hsa-miR-6511a-5p, miR-4525 is hsa-miR-4525, miR-1915-5p is hsa-miR-1915-5p, miR-3180 is hsa-miR-3180, miR-6879-5p is hsa-miR-6879-5p, miR-1199-5p is hsa-miR-1199-5p, miR-6746-5p is hsa-miR-6746-5p, miR-711 is hsa-miR-711, miR-663b is hsa-miR-663b, miR-4707-3p is hsa-miR-4707-3p, miR-6893-5p is hsa-miR-6893-5p, miR-4675 is hsa-miR-4675, miR-4638-5p is hsa-miR-4638-5p, miR-4651 is hsa-miR-4651, miR-6087 is hsa-miR-6087, miR-4665-5p is hsa-miR-4665-5p, miR-4758-5p is hsa-miR-4758-5p, miR-6887-5p is hsa-miR-6887-5p, miR-3620-5p is hsa-miR-3620-5p, miR-1909-3p is hsa-miR-1909-3p, miR-7641 is hsa-miR-7641, miR-6724-5p is hsa-miR-6724-5p, miR-1343-3p is hsa-miR-1343-3p, miR-6780b-5p is hsa-miR-6780b-5p, miR-4484 is hsa-miR-4484, miR-4690-5p is hsa-miR-4690-5p, miR-4429 is hsa-miR-4429, miR-1227-5p is hsa-miR-1227-5p, miR-4725-3p is hsa-miR-4725-3p, miR-6861-5p is hsa-miR-6861-5p, miR-6812-5p is hsa-miR-6812-5p, miR-3197 is hsa-miR-3197, miR-8059 is hsa-miR-8059, miR-3185 is hsa-miR-3185, miR-4706 is hsa-miR-4706, miR-4497 is hsa-miR-4497, miR-3131 is hsa-miR-3131, miR-6806-5p is hsa-miR-6806-5p, miR-187-5p is hsa-miR-187-5p, miR-3180-3p is hsa-miR-3180-3p, miR-6848-5p is hsa-miR-6848-5p, miR-6820-5p is hsa-miR-6820-5p, miR-6800-5p is hsa-miR-6800-5p, miR-6717-5p is hsa-miR-6717-5p, miR-6795-5p is hsa-miR-6795-5p, miR-4632-5p is hsa-miR-4632-5p, miR-665 is hsa-miR-665, miR-6778-5p is hsa-miR-6778-5p, miR-3663-3p is hsa-miR-3663-3p, miR-4689 is hsa-miR-4689, miR-211-3p is hsa-miR-211-3p, miR-6511b-5p is hsa-miR-6511b-5p, miR-4750-5p is hsa-miR-4750-5p, miR-6126 is hsa-miR-6126, miR-614 is hsa-miR-614, miR-7110-5p is hsa-miR-7110-5p, miR-744-5p is hsa-miR-744-5p, miR-6769a-5p is hsa-miR-6769a-5p, miR-4792 is hsa-miR-4792, miR-5787 is hsa-miR-5787, miR-6798-5p is hsa-miR-6798-5p, miR-6781-5p is hsa-miR-6781-5p, miR-4419b is hsa-miR-4419b, miR-4446-3p is hsa-miR-4446-3p, miR-4259 is hsa-miR-4259, miR-5572 is hsa-miR-5572, miR-6075 is hsa-miR-6075, miR-296-3p is hsa-miR-296-3p, miR-6891-5p is hsa-miR-6891-5p, miR-4745-5p is hsa-miR-4745-5p, miR-6775-5p is hsa-miR-6775-5p, miR-6870-5p is hsa-miR-6870-5p, miR-920 is hsa-miR-920, miR-4530 is hsa-miR-4530, miR-6819-5p is hsa-miR-6819-5p, miR-6825-5p is hsa-miR-6825-5p, miR-7847-3p is hsa-miR-7847-3p, miR-6131 is hsa-miR-6131, miR-4433-3p is hsa-miR-4433-3p, miR-1228-5p is hsa-miR-1228-5p, miR-6743-5p is hsa-miR-6743-5p, miR-1268a is hsa-miR-1268a, miR-3917 is hsa-miR-3917, miR-6786-5p is hsa-miR-6786-5p, miR-3154 is hsa-miR-3154, miR-638 is hsa-miR-638, miR-6741-5p is hsa-miR-6741-5p, miR-6889-5p is hsa-miR-6889-5p, miR-6840-3p is hsa-miR-6840-3p, miR-6510-5p is hsa-miR-6510-5p, miR-3188 is hsa-miR-3188, miR-551b-5p is hsa-miR-551b-5p, miR-5001-5p is hsa-miR-5001-5p, miR-1268b is hsa-miR-1268b, miR-7107-5p is hsa-miR-7107-5p, miR-6824-5p is hsa-miR-6824-5p, miR-6732-5p is hsa-miR-6732-5p, miR-371a-5p is hsa-miR-371a-5p, miR-6794-5p is hsa-miR-6794-5p, miR-6779-5p is hsa-miR-6779-5p, miR-4271 is hsa-miR-4271, miR-5195-3p is hsa-miR-5195-3p, miR-6762-5p is hsa-miR-6762-5p, miR-939-5p is hsa-miR-939-5p, miR-1247-3p is hsa-miR-1247-3p, miR-6777-5p is hsa-miR-6777-5p, miR-6722-3p is hsa-miR-6722-3p, miR-3656 is hsa-miR-3656, miR-4688 is hsa-miR-4688, miR-3195 is hsa-miR-3195, miR-6766-5p is hsa-miR-6766-5p, miR-4447 is hsa-miR-4447, miR-4656 is hsa-miR-4656, miR-7108-5p is hsa-miR-7108-5p, miR-3191-3p is hsa-miR-3191-3p, miR-1273g-3p is hsa-miR-1273g-3p, miR-4463 is hsa-miR-4463, miR-2861 is hsa-miR-2861, miR-3196 is hsa-miR-3196, miR-6877-5p is hsa-miR-6877-5p, miR-3679-5p is hsa-miR-3679-5p, miR-4442 is hsa-miR-4442, miR-6789-5p is hsa-miR-6789-5p, miR-6782-5p is hsa-miR-6782-5p, miR-486-3p is hsa-miR-486-3p, miR-6085 is hsa-miR-6085, miR-4746-3p is hsa-miR-4746-3p, miR-619-5p is hsa-miR-619-5p, miR-937-5p is hsa-miR-937-5p, miR-6803-5p is hsa-miR-6803-5p, miR-4298 is hsa-miR-4298, miR-4454 is hsa-miR-4454, miR-4459 is hsa-miR-4459, miR-7150 is hsa-miR-7150, miR-6880-5p is hsa-miR-6880-5p, miR-4449 is hsa-miR-4449, miR-8063 is hsa-miR-8063, miR-4695-5p is hsa-miR-4695-5p, miR-6132 is hsa-miR-6132, miR-6829-5p is hsa-miR-6829-5p, miR-4486 is hsa-miR-4486, miR-6805-3p is hsa-miR-6805-3p, miR-6826-5p is hsa-miR-6826-5p, miR-4508 is hsa-miR-4508, miR-1343-5p is hsa-miR-1343-5p, miR-7114-5p is hsa-miR-7114-5p, miR-3622a-5p is hsa-miR-3622a-5p, miR-6765-5p is hsa-miR-6765-5p, miR-7845-5p is hsa-miR-7845-5p, miR-3960 is hsa-miR-3960, miR-6749-5p is hsa-miR-6749-5p, miR-1260b is hsa-miR-1260b, miR-6799-5p is hsa-miR-6799-5p, miR-4723-5p is hsa-miR-4723-5p, miR-6784-5p is hsa-miR-6784-5p, miR-5100 is hsa-miR-5100, miR-6769b-5p is hsa-miR-6769b-5p, miR-1207-5p is hsa-miR-1207-5p, miR-642a-3p is hsa-miR-642a-3p, miR-4505 is hsa-miR-4505, miR-4270 is hsa-miR-4270, miR-6721-5p is hsa-miR-6721-5p, miR-7111-5p is hsa-miR-7111-5p, miR-6791-5p is hsa-miR-6791-5p, miR-7109-5p is hsa-miR-7109-5p, miR-4258 is hsa-miR-4258, miR-6515-3p is hsa-miR-6515-3p, miR-6851-5p is hsa-miR-6851-5p, miR-6125 is hsa-miR-6125, miR-4749-5p is hsa-miR-4749-5p, miR-4726-5p is hsa-miR-4726-5p, miR-4513 is hsa-miR-4513, miR-6089 is hsa-miR-6089, miR-6816-5p is hsa-miR-6816-5p, miR-4466 is hsa-miR-4466, miR-4488 is hsa-miR-4488, miR-6752-5p is hsa-miR-6752-5p, and miR-4739 is hsa-miR-4739.

(13) The device according to (11) or (12), wherein the nucleic acid is a polynucleotide selected from the group consisting of the following polynucleotides (a) to (e):
(a) a polynucleotide consisting of a nucleotide sequence represented by any of SEQ ID NOs: 1 to 235 and 851 to 856 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 235 and 851 to 856 or a nucleotide sequence derived from the nucleotide sequence by the replacement of u with t,
(c) a polynucleotide consisting of a nucleotide sequence complementary to a nucleotide sequence represented by any of SEQ ID NOs: 1 to 235 and 851 to 856 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 235 and 851 to 856 or a nucleotide sequence derived from the nucleotide sequence by the replacement of u with t, and
(e) a polynucleotide hybridizing under stringent conditions to any of the polynucleotides (a) to (d).

(14) The device according to any of (11) to (13), wherein the device further comprises nucleic acid(s) capable of specifically binding to at least one polynucleotide selected from the group consisting of other breast cancer markers miR-760, miR-602, miR-423-5p, miR-92a-2-5p, miR-16-5p, miR-451a, miR-135a-3p, miR-486-5p, miR-4257, miR-92b-5p, miR-1915-3p, miR-718, miR-940, miR-296-5p, miR-23b-3p and miR-92a-3p.

(15) The device according to (14), wherein miR-760 is hsa-miR-760, miR-602 is hsa-miR-602, miR-423-5p is hsa-miR-423-5p, miR-92a-2-5p is hsa-miR-92a-2-5p, miR-16-5p is hsa-miR-16-5p, miR-451a is hsa-miR-451a, miR-135a-3p is hsa-miR-135a-3p, miR-486-5p is hsa-miR-486-5p, miR-4257 is hsa-miR-4257, miR-92b-5p is hsa-miR-92b-5p, miR-1915-3p is hsa-miR-1915-3p, miR-718 is hsa-miR-718, miR-940 is hsa-miR-940, miR-296-5p is hsa-miR-296-5p, miR-23b-3p is hsa-miR-23b-3p, and miR-92a-3p is hsa-miR-92a-3p.

(16) The device according to (14) or (15), wherein the nucleic acid is a polynucleotide selected from the group consisting of the following polynucleotides (f) to (j):
(f) a polynucleotide consisting of a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 236 to 251,
(h) a polynucleotide consisting of a nucleotide sequence complementary to a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 236 to 251 or a nucleotide sequence derived from the nucleotide sequence by the replacement of u with t, and
(j) a polynucleotide hybridizing under stringent conditions to any of the polynucleotides (f) to (i).

(17) The device according to any of (11) to (16), wherein the device further comprises nucleic acid(s) capable of specifically binding to at least one polynucleotide selected from the group consisting of other breast cancer markers miR-658, miR-6842-5p, miR-6124, miR-6765-3p, miR-7106-5p, miR-4534, miR-92b-3p, miR-3135b, miR-4687-3p, miR-762, miR-3619-3p, miR-4467, miR-557, miR-1237-5p, miR-1908-5p, miR-4286, miR-6885-5p and miR-6763-5p.

(18) The device according to (17), wherein miR-658 is hsa-miR-658, miR-6842-5p is hsa-miR-6842-5p, miR-6124 is hsa-miR-6124, miR-6765-3p is hsa-miR-6765-3p, miR-7106-5p is hsa-miR-7106-5p, miR-4534 is hsa-miR-4534, miR-92b-3p is hsa-miR-92b-3p, miR-3135b is hsa-miR-3135b, miR-4687-3p is hsa-miR-4687-3p, miR-762 is hsa-miR-762, miR-3619-3p is hsa-miR-3619-3p, miR-4467 is hsa-miR-4467, miR-557 is hsa-miR-557, miR-1237-5p is hsa-miR-1237-5p, miR-1908-5p is hsa-miR-1908-5p, miR-4286 is hsa-miR-4286, miR-6885-5p is hsa-miR-6885-5p, and miR-6763-5p is hsa-miR-6763-5p.

(19) The device according to (17) or (18), wherein the nucleic acid is a polynucleotide selected from the group consisting of the following polynucleotides (k) to (o):
(k) a polynucleotide consisting of a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 252 to 269,
(m) a polynucleotide consisting of a nucleotide sequence complementary to a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 252 to 269 or a nucleotide sequence derived from the nucleotide sequence by the replacement of u with t, and
(o) a polynucleotide hybridizing under stringent conditions to any of the polynucleotides (k) to (n).

(20) The device according to any one of (11) to (19), wherein the device is for measurement based on a hybridization technique.

(21) The device according to (20), wherein the hybridization technique is a nucleic acid array technique.

(22) The device according to any one of (11) to (21), wherein the device comprises at least two nucleic acids capable of specifically binding to at least two polynucleotides, respectively, selected from all of the breast cancer markers according to (11) or (12).

(23) A method for detecting breast cancer, comprising measuring an expression level(s) of a target nucleic acid(s) in a sample from a subject using a kit according to any one of (1) to (10) or a device according to any one of (11) to (22), and evaluating in vitro whether or not the subject has breast cancer using both of the measured expression level(s) and a control expression level(s) in a sample from a healthy subject measured in the same way.

(24) The method according to (23), wherein the subject is a human.

(25) The method according to (23) or (24), wherein the sample is blood, serum, or plasma.

### DEFINITION OF TERMS

The terms used herein are defined as described below.

Abbreviations or terms such as nucleotide, polynucleotide, DNA, and RNA used herein abide by “Guidelines for the preparation of specification which contain nucleotide and/or amino acid sequences” (edited by Japan Patent Office) and common use in the art.

The term “polynucleotide” used herein refers to a nucleic acid including any of RNA, DNA, and RNA/DNA (chimera). The DNA includes any of cDNA, genomic DNA, and synthetic DNA. The RNA includes any of total RNA, mRNA, rRNA, miRNA, siRNA, snoRNA, snRNA, non-coding RNA and synthetic RNA. The “synthetic DNA” and the “synthetic RNA” used herein refer to DNA and RNA artificially prepared using, for example, an automatic nucleic acid synthesizer, on the basis of predetermined nucleotide sequences (which may be any of natural and non-natural sequences). The “non-natural sequence” used herein is intended to be used in a broad sense and includes, for example, a sequence comprising substitution, deletion, insertion, and/or addition of one or more nucleotide(s) (i.e., a variant sequence) and a sequence comprising one or more modified nucleotide(s) (i.e., a modified sequence), which are different from the natural sequence. The term “polynucleotide” used herein is used interchangeably with the term “nucleic acid”.

The term “fragment” used herein refers to a polynucleotide (including oligonucleotides) having a nucleotide sequence that consists of a consecutive portion of a polynucleotide and desirably has a length of 15 or more nucleotides, preferably 17 or more nucleotides, more preferably 19 or more nucleotides.

The term “gene” used herein is intended to include not only RNA and double-stranded DNA but also each single-stranded DNA such as a plus strand (or a sense strand) or a complementary strand (or an antisense strand) constituting the duplex. The gene is not particularly limited by its length. Thus, The “gene” used herein includes any of double-stranded DNA including human genomic DNA, single-stranded DNA (plus strand), single-stranded DNA having a sequence complementary to the plus strand (complementary strand) including cDNA, microRNA (miRNA), and their fragments, and their transcripts, unless otherwise specified. The “gene” includes not only a “gene” represented by a particular nucleotide sequence (or SEQ ID NO) but also “nucleic acids” encoding RNAs having biological functions equivalent to an RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 871 or a nucleotide sequence derived from the nucleotide sequence by the replacement of u with t. Regardless whether or not there is a difference in functional region, the “gene(s)” can comprise, for example, expression regulatory region(s), coding region(s), exon(s), or intron(s). The “gene” may be contained in a cell or may exist alone after being released into the outside of a cell. Alternatively, the “gene” may be in a state enclosed in a vesicle called exosome.

The term “exosome” used herein refers to a vesicle that is encapsulated by a lipid bilayer and secreted from a cell. The exosome is derived from a multivesicular endosome and may incorporate biomaterials such as “gene(s)” (e.g., RNA or DNA) or protein(s) when released into an extracellular environment. The exosome is known to be contained in a body fluid such as blood, serum, plasma, or lymph.

The term “transcript” used herein refers to RNA synthesized with the DNA sequence of a gene as a template. RNA polymerase binds to a site called a promoter located upstream of the gene and adds ribonucleotides complementary to the nucleotide sequence of the DNA to the 3′ end to synthesize an RNA. This RNA contains not only the gene itself but also the whole sequence from a transcription initiation site to the end of a polyA sequence, including expression regultory region(s), coding region(s), exon(s), or intron(s).

Unless otherwise specified, the term “microRNA (miRNA)” used herein is intended to mean a 15- to 25-nucleotide non-coding RNA that is transcribed as an RNA precursor which has a hairpin-like structure, cleaved by a dsRNA-cleaving enzyme which has RNase III cleavage activity, and integrated into a protein complex called RISC, and is involved in the suppression of translation of mRNA. The term “miRNA” used herein includes not only a “miRNA” represented by a particular nucleotide sequence (or SEQ ID NO) but also a precursor of the “miRNA” (pre-miRNA or pri-miRNA), and miRNAs that have biological functions equivalent thereto, for example, a congener (i.e., a homolog or an ortholog), a variant (e.g., a genetic polymorph), and a derivative. Such a precursor, a congener, a variant, or a derivative can be specifically identified using miRBase Release 20 (http://www.mirbase.org/), and examples thereof can include a “miRNA” having a nucleotide sequence hybridizing under stringent conditions described later to a complementary sequence of any particular nucleotide sequence represented by any of SEQ ID NOs: 1 to 871. The term “miRNA” used herein may be a gene product of a miR gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA as described above).

The term “probe” used herein includes a polynucleotide that is used for specifically detecting an RNA resulting from the expression of a gene or a polynucleotide derived from the RNA, and/or a polynucleotide complementary thereto.

The term “primer” used herein includes a polynucleotide that specifically recognizes and amplifies RNA resulting from the expression of a gene or a polynucleotide derived from the RNA, and/or a polynucleotide complementary thereto. In this context, the “complementary polynucleotide (complementary strand or reverse strand)” means a polynucleotide in a complementary relationship of A:T (U) and G:C base pairs with the full-length sequence of a polynucleotide consisting of a nucleotide sequence defined by any of SEQ ID NOs: 1 to 871 or a nucleotide sequence derived from the nucleotide sequence by the replacement of u with t, or a partial sequence thereof (here, this full-length or partial sequence is referred to as a plus strand for the sake of convenience). However, such a complementary strand is not limited to a sequence completely complementary to the nucleotide sequence of the target plus strand and may have a complementary relationship to an extent that permits hybridization under stringent conditions to the target plus strand.

The term “stringent conditions” used herein refers to conditions under which a nucleic acid probe hybridizes to its target sequence to a larger extent (e.g., a measurement value equal to or larger than “(a mean of background measurement values)+(a standard deviation of the background measurement values)×2”) than that for other sequences. The stringent conditions are dependent on a sequence and differ depending on an environment where hybridization is performed. A target sequence complementary 100% to the nucleic acid probe can be identified by controlling the stringency of hybridization and/or washing conditions. Specific examples of the “stringent conditions” will be mentioned later.

The term “Tm value” used herein means a temperature at which the double-stranded moiety of a polynucleotide is denatured into single strands so that the double strands and the single strands exist at a ratio of 1:1.

The term “variant” used herein means, in the case of a nucleic acid, a natural variant attributed to polymorphism, mutation, or the like; a variant containing the deletion, substitution, addition, or insertion of 1 or 2 or more nucleotides in a nucleotide sequence represented by any of SEQ ID NOs: 1 to 871 or a nucleotide sequence derived from the nucleotide sequence by the replacement of u with t, or a partial sequence thereof; a variant that exhibits percent (%) identity of approximately 90% or higher, approximately 95% or higher, approximately 97% or higher, approximately 98% or higher, approximately 99% or higher to each of these nucleotide sequences or the partial sequence thereof; or a nucleic acid hybridizing under the stringent conditions defined above to a polynucleotide or an oligonucleotide comprising each of these nucleotide sequences or the partial sequence thereof.

The term “multiple” used herein means an integer of approximately 10, 9, 8, 7, 6, 5, 4, 3, or 2.

The “variant” used herein can be prepared by use of a well-known technique such as site-directed mutagenesis or PCR-based mutagenesis.

The term “percent (%) identity” used herein can be determined with or without an introduced gap, using a protein or gene search system based on BLAST or FASTA described above (Zheng Zhang et al., 2000, J. Comput. Biol., Vol. 7, p. 203-214; Altschul, S. F. et al., 1990, Journal of Molecular Biology, Vol. 215, p. 403-410; and Pearson, W. R. et al., 1988, Proc. Natl. Acad. Sci. U.S.A., Vol. 85, p. 2444-2448).

The term “derivative” used herein is meant to include a modified nucleic acid, for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen, P. E. et al., 1991, Science, Vol. 254, p. 1497-500), and LNA (locked nucleic acid; Obika, S. et al., 1998, Tetrahedron Lett., Vol. 39, p. 5401-5404) without any limitation.

The “nucleic acid” used herein capable of specifically binding to a polynucleotide selected from the breast cancer marker miRNAs described above is a synthesized or prepared nucleic acid and specifically includes a “nucleic acid probe” or a “primer”. The “nucleic acid” is utilized directly or indirectly for detecting the presence or absence of breast cancer in a subject, for diagnosing the presence or absence of breast cancer, or the severity of breast cancer, the presence or absence of amelioration of breast cancer, or the degree of amelioration of breast cancer, or the therapeutic sensitivity of breast cancer, or for screening for a candidate substance useful in the prevention, amelioration, or treatment of breast cancer. The “nucleic acid” includes a nucleotide, an oligonucleotide, and a polynucleotide capable of specifically recognizing and binding to a transcript represented by any of SEQ ID NOs:1 to 871 or a synthetic cDNA nucleic acid thereof in vivo, particularly, in a sample such as a body fluid (e.g., blood or urine), in relation to the development of breast cancer. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” used herein is interchangeable with the term “examination”, “measurement”, or “detection or decision support”. The term “evaluation” used herein is meant to include diagnosing or evaluation-supporting on the basis of examination results or measurement results.

The term “subject” used herein means a mammal such as a primate including a human and a chimpanzee, a pet animal including a dog and a cat, a livestock animal including cattle, a horse, sheep, and a goat, and a rodent including a mouse and a rat. The term “healthy subject” also means such a mammal without the cancer to be detected.

The term “P” or “P value” used herein refers to a probability at which a more extreme statistic than that is actually calculated from data under a null hypothesis is observed in a statistical test. Thus, smaller “P” or “P value” is regarded as being a more significant difference between subjects to be compared.

The term “sensitivity” used herein means a value of (the number of true positives)/(the number of true positives+the number of false negatives). High sensitivity allows breast cancer to be detected early, leading to the complete resection of cancer sites and reduction in the rate of recurrence.

The term “specificity” used herein means a value of (the number of true negatives)/(the number of true negatives+the number of false positives). High specificity prevents needless extra examination for healthy subjects who might have been misjudged as being breast cancer patients, leading to reduction in burden on patients and reduction in medical expense.

The term “accuracy” used herein means a value of (the number of true positives+the number of true negatives)/(the total number of cases). The accuracy indicates the ratio of samples that are identified correctly to all samples, and serves as a primary index for evaluating detection performance.

The “sample” used herein, that is subjected to determination, detection, or diagnosis refers to a tissue and a biological material in which the expression of the gene of the present invention varies as breast cancer develops, as breast cancer progresses, or as therapeutic effects on breast cancer are exerted. Specifically, the “sample” refers to a breast tissue, a perimammary vascular channel, lymph node, and organ, an organ suspected of having metastasis, the skin, a body fluid such as blood, urine, saliva, sweat, or tissue exudates, serum or plasma prepared from blood, feces, hair, and the like. The “sample” further refers to a biological sample extracted therefrom, specifically, a gene such as RNA or miRNA.

The term “hsa-miR-4783-3p gene” or “hsa-miR-4783-3p” used herein includes the hsa-miR-4783-3p gene (miRBase Accession No. MIMAT0019947) described in SEQ ID NO: 1, a homolog or an ortholog of a different organism species, and the like. The hsa-miR-4783-3p gene can be obtained by a method described in Persson H et al., 2011, Cancer Res, Vol. 71, p. 78-86. Also, “hsa-mir-4783” (miRBase Accession No. MI0017428, SEQ ID NO: 270) having a hairpin-like structure is known as a precursor of “hsa-miR-4783-3p”.

The term “hsa-miR-4730 gene” or “hsa-miR-4730” used herein includes the hsa-miR-4730 gene (miRBase Accession No. MIMAT0019852) described in SEQ ID NO: 2, a homolog or an ortholog of a different organism species, and the like. The hsa-miR-4730 gene can be obtained by a method described in Persson H et al., 2011, Cancer Res, Vol. 71, p. 78-86. Also, “hsa-mir-4730” (miRBase Accession No. MI0017367, SEQ ID NO: 271) having a hairpin-like structure is known as a precursor of “hsa-miR-4730”.

The term “hsa-miR-1307-3p gene” or “hsa-miR-1307-3p” used herein includes the hsa-miR-1307-3p gene (miRBase Accession No. MIMAT0005951) described in SEQ ID NO: 3, a homolog or an ortholog of a different organism species, and the like. The hsa-miR-1307-3p gene can be obtained by a method described in Morin R D et al., 2008, Genome Res, Vol. 18, p. 610-621. Also, “hsa-mir-1307” (miRBase Accession No. MI0006444, SEQ ID NO: 272) having a hairpin-like structure is known as a precursor of “hsa-miR-1307-3p”.

The term “hsa-miR-4634 gene” or “hsa-miR-4634” used herein includes the hsa-miR-4634 gene (miRBase Accession No. MIMAT0019691) described in SEQ ID NO: 4, a homolog or an ortholog of a different organism species, and the like. The hsa-miR-4634 gene can be obtained by a method described in Persson H et al., 2011, Cancer Res, Vol. 71, p. 78-86. Also, “hsa-mir-4634” (miRBase Accession No. MI0017261, SEQ ID NO: 273) having a hairpin-like structure is known as a precursor of “hsa-miR-4634”.

The term “hsa-miR-663a gene” or “hsa-miR-663a” used herein includes the hsa-miR-663a gene (miRBase Accession No. MIMAT0003326) described in SEQ ID NO: 5, a homolog or an ortholog of a different organism species, and the like. The hsa-miR-663a gene can be obtained by a method described in Cummins J M et al., 2006, Proc Natl Acad Sci USA, Vol. 103, p. 3687-3692. Also, “hsa-mir-663a” (miRBase Accession No. MI0003672, SEQ ID NO: 274) having a hairpin-like structure is known as a precursor of “hsa-miR-663a”.

The term “hsa-miR-4532 gene” or “hsa-miR-4532” used herein includes the hsa-miR-4532 gene (miRBase Accession No. MIMAT0019071) described in SEQ ID NO: 6, a homolog or an ortholog of a different organism species, and the like. The hsa-miR-4532 gene can be obtained by a method described in Jima D D et al., 2010, Blood, Vol. 116, e118-e127. Also, “hsa-mir-4532” (miRBase Accession No. MI0016899, SEQ ID NO: 275) having a hairpin-like structure is known as a precursor of “hsa-miR-4532”.

The term “hsa-miR-7704 gene” or “hsa-miR-7704” used herein includes the hsa-miR-7704 gene (miRBase Accession No. MIMAT0030019) described in SEQ ID NO: 7, a homolog or an ortholog of a different organism species, and the like. The hsa-miR-7704 gene can be obtained by a method described in Swaminathan S et al., 2013, Biochem Biophys Res Commun, Vol. 434, p. 228-234. Also, “hsa-mir-7704” (miRBase Accession No. MI0025240, SEQ ID NO: 276) having a hairpin-like structure is known as a precursor of “hsa-miR-7704”.

The term “hsa-miR-3178 gene” or “hsa-miR-3178” used herein includes the hsa-miR-3178 gene (miRBase Accession No. MIMAT0015055) described in SEQ ID NO: 8, a homolog or an ortholog of a different organism species, and the like. The hsa-miR-3178 gene can be obtained by a method described in Stark M S et al., 2010, PLoS One, Vol. 5, e9685. Also, “hsa-mir-3178” (miRBase Accession No. MI0014212, SEQ ID NO: 277) having a hairpin-like structure is known as a precursor of “hsa-miR-3178”.

The term “hsa-miR-6729-5p gene” or “hsa-miR-6729-5p” used herein includes the hsa-miR-6729-5p gene (miRBase Accession No. MIMAT0027359) described in SEQ ID NO: 9, a homolog or an ortholog of a different organism species, and the like. The hsa-miR-6729-5p gene can be obtained by a method described in Ladewig E et al., 2012, Genome Res, Vol. 22, p. 1634-1645. Also, “hsa-mir-6729” (miRBase Accession No. MI0022574, SEQ ID NO: 278) having a hairpin-like structure is known as a precursor of “hsa-miR-6729-5p”.

The term “hsa-miR-6090 gene” or “hsa-miR-6090” used in herein includes the hsa-miR-6090 gene (miRBase Accession No. MIMAT0023715) described in SEQ ID NO: 10, a homolog or an ortholog of a different organism species, and the like. The hsa-miR-6090 gene can be obtained by a method described in Yoo J K et al., 2012, Stem Cells Dev, Vol. 21, p. 2049-2057. Also, “hsa-mir-6090” (miRBase Accession No. MI0020367, SEQ ID NO: 279) having a hairpin-like structure is known as a precursor of “hsa-miR-6090”.

The term “hsa-miR-4732-5p gene” or “hsa-miR-4732-5p” used in herein includes the hsa-miR-4732-5p gene (miRBase Accession No. MIMAT0019855) described in SEQ ID NO: 11, a homolog or an ortholog of a different organism species, and the like. The hsa-miR-4732-5p gene can be obtained by a method described in Persson H et al., 2011, Cancer Res, Vol. 71, p. 78-86. Also, “hsa-mir-4732” (miRBase Accession No. MI0017369, SEQ ID NO: 280) having a hairpin-like structure is known as a precursor of “hsa-miR-4732-5p”.

The term “hsa-miR-3184-5p gene” or “hsa-miR-3184-5p” used herein includes the hsa-miR-3184-5p gene (miRBase Accession No. MIMAT0015064) described in SEQ ID NO: 12, a homolog or an ortholog of a different organism species, and the like. The hsa-miR-3184-5p gene can be obtained by a method described in Stark M S et al., 2010, PLoS One, Vol. 5, e9685. Also, “hsa-mir-3184” (miRBase Accession No. MI0014226, SEQ ID NO: 281) having a hairpin-like structure is known as a precursor of “hsa-miR-3184-5p”.

The term “hsa-miR-6727-5p gene” or “hsa-miR-6727-5p” used herein includes the hsa-miR-6727-5p gene (miRBase Accession No. MIMAT0027355) described in SEQ ID NO: 13, a homolog or an ortholog of a different organism species, and the like. The hsa-miR-6727-5p gene can be obtained by a method described in Ladewig E et al., 2012, Genome Res, Vol. 22, p. 1634-1645. Also, “hsa-mir-6727” (miRBase Accession No. MI0022572, SEQ ID NO: 282) having a hairpin-like structure is known as a precursor of “hsa-miR-6727-5p”.

The term “hsa-miR-6088 gene” or “hsa-miR-6088” used herein includes the hsa-miR-6088 gene (miRBase Accession No. MIMAT0023713) described in SEQ ID NO: 14, a homolog or an ortholog of a different organism species, and the like. The hsa-miR-6088 gene can be obtained by a method described in Yoo J K et al., 2012, Stem Cells Dev, Vol. 21, p. 2049-2057. Also, “hsa-mir-6088” (miRBase Accession No. MI0020365, SEQ ID NO: 283) having a hairpin-like structure is known as a precursor of “hsa-miR-6088”.

The term “hsa-miR-4674 gene” or “hsa-miR-4674” used herein includes the hsa-miR-4674 gene (miRBase Accession No. MIMAT0019756) described in SEQ ID NO: 15, a homolog or an ortholog of a different organism species, and the like. The hsa-miR-4674 gene can be obtained by a method described in Persson H et al., 2011, Cancer Res, Vol. 71, p. 78-86. Also, “hsa-mir-4674” (miRBase Accession No. MI0017305, SEQ ID NO: 284) having a hairpin-like structure is known as a precursor of “hsa-miR-4674”.

The term “hsa-miR-8073 gene” or “hsa-miR-8073” used herein includes the hsa-miR-8073 gene (miRBase Accession No. MIMAT0031000) described in SEQ ID NO: 16, a homolog or an ortholog of a different organism species, and the like. The hsa-miR-8073 gene can be obtained by a method described in Wang H J et al., 2013, Shock, Vol. 39, p. 480-487. Also, “hsa-mir-8073” (miRBase Accession No. MI0025909, SEQ ID NO: 285) having a hairpin-like structure is known as a precursor of “hsa-miR-8073”.

The term “hsa-miR-4787-5p gene” or “hsa-miR-4787-5p” used herein includes the hsa-miR-4787-5p gene (miRBase Accession No. MIMAT0019956) described in SEQ ID NO: 17, a homolog or an ortholog of a different organism species, and the like. The hsa-miR-4787-5p gene can be obtained by a method described in Persson H et al., 2011, Cancer Res, Vol. 71, p. 78-86. Also, “hsa-mir-4787” (miRBase Accession No. MI0017434, SEQ ID NO: 286) having a hairpin-like structure is known as a precursor of “hsa-miR-4787-5p”.

The term “hsa-miR-1469 gene” or “hsa-miR-1469” used herein includes the hsa-miR-1469 gene (miRBase Accession No. MIMAT0007347) described in SEQ ID NO: 18, a homolog or an ortholog of a different organism species, and the like. The hsa-miR-1469 gene can be obtained by a method described in Kawaji H et al., 2008, BMC Genomics, Vol. 9, p. 157. Also, “hsa-mir-1469” (miRBase Accession No. MI0007074, SEQ ID NO: 287) having a hairpin-like structure is known as a precursor of “hsa-miR-1469”.

The term “hsa-miR-125a-3p gene” or “hsa-miR-125a-3p” used herein includes the hsa-miR-125a-3p gene (miRBase Accession No. MIMAT0004602) described in SEQ ID NO: 19, a homolog or an ortholog of a different organism species, and the like. The hsa-miR-125a-3p gene can be obtained by a method described in Lagos-Quintana M et al., 2002, Curr Biol, Vol. 12, p. 735-739. Also, “hsa-mir-125a” (miRBase Accession No. MI0000469, SEQ ID NO: 288) having a hairpin-like structure is known as a precursor of “hsa-miR-125a-3p”.

The term “hsa-miR-1233-5p gene” or “hsa-miR-1233-5p” used herein includes the hsa-miR-1233-5p gene (miRBase Accession No. MIMAT0022943) described in SEQ ID NO: 20, a homolog or an ortholog of a different organism species, and the like. The hsa-miR-1233-5p gene can be obtained by a method described in Berezikov E et al., 2007, Mol Cell, Vol. 28, p. 328-336. Also, “hsa-mir-1233-1 and hsa-mir-1233-2” (miRBase Accession Nos. MI0006323 and MI0015973, SEQ ID NOs: 289 and 290) having a hairpin-like structure are known as precursors of “hsa-miR-1233-5p”.

The term “hsa-miR-885-3p gene” or “hsa-miR-885-3p” used herein includes the hsa-miR-885-3p gene (miRBase Accession No. MIMAT0004948) described in SEQ ID NO: 21, a homolog or an ortholog of a different organism species, and the like. The hsa-miR-885-3p gene can be obtained by a method described in Berezikov E et al., 2006, Genome Res, Vol. 16, p. 1289-1298. Also, “hsa-mir-885” (miRBase Accession No. MI0005560, SEQ ID NO: 291) having a hairpin-like structure is known as a precursor of “hsa-miR-885-3p”.

The term “hsa-miR-6802-5p gene” or “hsa-miR-6802-5p” used herein includes the hsa-miR-6802-5p gene (miRBase Accession No. MIMAT0027504) described in SEQ ID NO: 22, a homolog or an ortholog of a different organism species, and the like. The hsa-miR-6802-5p gene can be obtained by a method described in Ladewig E et al., 2012, Genome Res, Vol. 22, p. 1634-1645. Also, “hsa-mir-6802” (miRBase Accession No. MI0022647, SEQ ID NO: 292) having a hairpin-like structure is known as a precursor of “hsa-miR-6802-5p”.

The term “hsa-miR-328-5p gene” or “hsa-miR-328-5p” used herein includes the hsa-miR-328-5p gene (miRBase Accession No. MIMAT0026486) described in SEQ ID NO: 23, a homolog or an ortholog of a different organism species, and the like. The hsa-miR-328-5p gene can be obtained by a method described in Kim J et al., 2004, Proc Natl Acad Sci USA, Vol. 101, p. 360-365. Also, “hsa-mir-328” (miRBase Accession No. MI0000804, SEQ ID NO: 293) having a hairpin-like structure is known as a precursor of “hsa-miR-328-5p”.

The term “hsa-miR-6787-5p gene” or “hsa-miR-6787-5p” used herein includes the hsa-miR-6787-5p gene (miRBase Accession No. MIMAT0027474) described in SEQ ID NO: 24, a homolog or an ortholog of a different organism species, and the like. The hsa-miR-6787-5p gene can be obtained by a method described in Ladewig E et al., 2012, Genome Res, Vol. 22, p. 1634-1645. Also, “hsa-mir-6787” (miRBase Accession No. MI0022632, SEQ ID NO: 294) having a hairpin-like structure is known as a precursor of “hsa-miR-6787-5p”.

The term “hsa-miR-8069 gene” or “hsa-miR-8069” used herein includes the hsa-miR-8069 gene (miRBase Accession No. MIMAT0030996) described in SEQ ID NO: 25, a homolog or an ortholog of a different organism species, and the like. The hsa-miR-8069 gene can be obtained by a method described in Wang H J et al., 2013, Shock, Vol. 39, p. 480-487. Also, “hsa-mir-8069” (miRBase Accession No. MI0025905, SEQ ID NO: 295) having a hairpin-like structure is known as a precursor of “hsa-miR-8069”.

The term “hsa-miR-6875-5p gene” or “hsa-miR-6875-5p” used herein includes the hsa-miR-6875-5p gene (miRBase Accession No. MIMAT0027650) described in SEQ ID NO: 26, a homolog or an ortholog of a different organism species, and the like. The hsa-miR-6875-5p gene can be obtained by a method described in Ladewig E et al., 2012, Genome Res, Vol. 22, p. 1634-1645. Also, “hsa-mir-6875” (miRBase Accession No. MI0022722, SEQ ID NO: 296) having a hairpin-like structure is known as a precursor of “hsa-miR-6875-5p”.

The term “hsa-miR-1246 gene” or “hsa-miR-1246” used herein includes the hsa-miR-1246 gene (miRBase Accession No. MIMAT0005898) described in SEQ ID NO: 27, a homolog or an ortholog of a different organism species, and the like. The hsa-miR-1246 gene can be obtained by a method described in Morin R D et al., 2008, Genome Res, Vol. 18, p. 610-621. Also, “hsa-mir-1246” (miRBase Accession No. MI0006381, SEQ ID NO: 297) having a hairpin-like structure is known as a precursor of “hsa-miR-1246”.

The term “hsa-miR-4734 gene” or “hsa-miR-4734” used herein includes the hsa-miR-4734 gene (miRBase Accession No. MIMAT0019859) described in SEQ ID NO: 28, a homolog or an ortholog of a different organism species, and the like. The hsa-miR-4734 gene can be obtained by a method described in Persson H et al., 2011, Cancer Res, Vol. 71, p. 78-86. Also, “hsa-mir-4734” (miRBase Accession No. MI0017371, SEQ ID NO: 298) having a hairpin-like structure is known as a precursor of “hsa-miR-4734”.

The term “hsa-miR-6757-5p gene” or “hsa-miR-6757-5p” used herein includes the hsa-miR-6757-5p gene (miRBase Accession No. MIMAT0027414) described in SEQ ID NO: 29, a homolog or an ortholog of a different organism species, and the like. The hsa-miR-6757-5p gene can be obtained by a method described in Ladewig E et al., 2012, Genome Res, Vol. 22, p. 1634-1645. Also, “hsa-mir-6757” (miRBase Accession No. MI0022602, SEQ ID NO: 299) having a hairpin-like structure is known as a precursor of “hsa-miR-6757-5p”.

The term “hsa-miR-6756-5p gene” or “hsa-miR-6756-5p” used herein includes the hsa-miR-6756-5p gene (miRBase Accession No. MIMAT0027412) described in SEQ ID NO: 30, a homolog or an ortholog of a different organism species, and the like. The hsa-miR-6756-5p gene can be obtained by a method described in Ladewig E et al., 2012, Genome Res, Vol. 22, p. 1634-1645. Also, “hsa-mir-6756” (miRBase Accession No. MI0022601, SEQ ID NO: 300) having a hairpin-like structure is known as a precursor of “hsa-miR-6756-5p”.

The term “hsa-miR-3665 gene” or “hsa-miR-3665” used herein includes the hsa-miR-3665 gene (miRBase Accession No. MIMAT0018087) described in SEQ ID NO: 31, a homolog or an ortholog of a different organism species, and the like. The hsa-miR-3665 gene can be obtained by a method described in Xie X et al., 2005, Nature, Vol. 434, p. 338-345. Also, “hsa-mir-3665” (miRBase Accession No. MI0016066, SEQ ID NO: 301) having a hairpin-like structure is known as a precursor of “hsa-miR-3665”.

The term “hsa-miR-6836-3p gene” or “hsa-miR-6836-3p” used herein includes the hsa-miR-6836-3p gene (miRBase Accession No. MIMAT0027575) described in SEQ ID NO: 32, a homolog or an ortholog of a different organism species, and the like. The hsa-miR-6836-3p gene can be obtained by a method described in Ladewig E et al., 2012, Genome Res, Vol. 22, p. 1634-1645. Also, “hsa-mir-6836” (miRBase Accession No. MI0022682, SEQ ID NO: 302) having a hairpin-like structure is known as a precursor of “hsa-miR-6836-3p”.

The term “hsa-miR-6821-5p gene” or “hsa-miR-6821-5p” used herein includes the hsa-miR-6821-5p gene (miRBase Accession No. MIMAT0027542) described in SEQ ID NO: 33, a homolog or an ortholog of a different organism species, and the like. The hsa-miR-6821-5p gene can be obtained by a method described in Ladewig E et al., 2012, Genome Res, Vol. 22, p. 1634-1645. Also, “hsa-mir-6821” (miRBase Accession No. MI0022666, SEQ ID NO: 303) having a hairpin-like structure is known as a precursor of “hsa-miR-6821-5p”.

The term “hsa-miR-6805-5p gene” or “hsa-miR-6805-5p” used herein includes the hsa-miR-6805-5p gene (miRBase Accession No. MIMAT0027510) described in SEQ ID NO: 34, a homolog or an ortholog of a different organism species, and the like. The hsa-miR-6805-5p gene can be obtained by a method described in Ladewig E et al., 2012, Genome Res, Vol. 22, p. 1634-1645. Also, “hsa-mir-6805” (miRBase Accession No. MI0022650, SEQ ID NO: 304) having a hairpin-like structure is known as a precursor of “hsa-miR-6805-5p”.

The term “hsa-miR-4728-5p gene” or “hsa-miR-4728-5p” used herein includes the hsa-miR-4728-5p gene (miRBase Accession No. MIMAT0019849) described in SEQ ID NO: 35, a homolog or an ortholog of a different organism species, and the like. The hsa-miR-4728-5p gene can be obtained by a method described in Persson H et al., 2011, Cancer Res, Vol. 71, p. 78-86. Also, “hsa-mir-4728” (miRBase Accession No. MI0017365, SEQ ID NO: 305) having a hairpin-like structure is known as a precursor of “hsa-miR-4728-5p”.

The term “hsa-miR-6726-5p gene” or “hsa-miR-6726-5p” used herein includes the hsa-miR-6726-5p gene (miRBase Accession No. MIMAT0027353) described in SEQ ID NO: 36, a homolog or an ortholog of a different organism species, and the like. The hsa-miR-6726-5p gene can be obtained by a method described in Ladewig E et al., 2012, Genome Res, Vol. 22, p. 1634-1645. Also, “hsa-mir-6726” (miRBase Accession No. MI0022571, SEQ ID NO: 306) having a hairpin-like structure is known as a precursor of “hsa-miR-6726-5p”.

The term “hsa-miR-197-5p gene” or “hsa-miR-197-5p” used herein includes the hsa-miR-197-5p gene (miRBase Accession No. MIMAT0022691) described in SEQ ID NO: 37, a homolog or an ortholog of a different organism species, and the like. The hsa-miR-197-5p gene can be obtained by a method described in Lagos-Quintana M et al., 2003, RNA, Vol. 9, p. 175-179. Also, “hsa-mir-197” (miRBase Accession No. MI0000239, SEQ ID NO: 307) having a hairpin-like structure is known as a precursor of “hsa-miR-197-5p”.

The term “hsa-miR-149-3p gene” or “hsa-miR-149-3p” used herein includes the hsa-miR-149-3p gene (miRBase Accession No. MIMAT0004609) described in SEQ ID NO: 38, a homolog or an ortholog of a different organism species, and the like. The hsa-miR-149-3p gene can be obtained by a method described in Lagos-Quintana M et al., 2002, Curr Biol, Vol. 12, p. 735-739. Also, “hsa-mir-149” (miRBase Accession No. MI0000478, SEQ ID NO: 308) having a hairpin-like structure is known as a precursor of “hsa-miR-149-3p”.

The term “hsa-miR-6850-5p gene” or “hsa-miR-6850-5p” used herein includes the hsa-miR-6850-5p gene (miRBase Accession No. MIMAT0027600) described in SEQ ID NO: 39, a homolog or an ortholog of a different organism species, and the like. The hsa-miR-6850-5p gene can be obtained by a method described in Ladewig E et al., 2012, Genome Res, Vol. 22, p. 1634-1645. Also, “hsa-mir-6850” (miRBase Accession No. MI0022696, SEQ ID NO: 309) having a hairpin-like structure is known as a precursor of “hsa-miR-6850-5p”.

The term “hsa-miR-4476 gene” or “hsa-miR-4476” used herein includes the hsa-miR-4476 gene (miRBase Accession No. MIMAT0019003) described in SEQ ID NO: 40, a homolog or an ortholog of a different organism species, and the like. The hsa-miR-4476 gene can be obtained by a method described in lima D D et al., 2010, Blood, Vol. 116, e118-e127. Also, “hsa-mir-4476” (miRBase Accession No. MI0016828, SEQ ID NO: 310) having a hairpin-like structure is known as a precursor of “hsa-miR-4476”.

The term “hsa-miR-6858-5p gene” or “hsa-miR-6858-5p” used herein includes the hsa-miR-6858-5p gene (miRBase Accession No. MIMAT0027616) described in SEQ ID NO: 41, a homolog or an ortholog of a different organism species, and the like. The hsa-miR-6858-5p gene can be obtained by a method described in Ladewig E et al., 2012, Genome Res, Vol. 22, p. 1634-1645. Also, “hsa-mir-6858” (miRBase Accession No. MI0022704, SEQ ID NO: 311) having a hairpin-like structure is known as a precursor of “hsa-miR-6858-5p”.

The term “hsa-miR-564 gene” or “hsa-miR-564” used herein includes the hsa-miR-564 gene (miRBase Accession No. MIMAT0003228) described in SEQ ID NO: 42, a homolog or an ortholog of a different organism species, and the like. The hsa-miR-564 gene can be obtained by a method described in Cummins J M et al., 2006, Proc Natl Acad Sci USA, Vol. 103, p. 3687-3692. Also, “hsa-mir-564” (miRBase Accession No. MI0003570, SEQ ID NO: 312) having a hairpin-like structure is known as a precursor of “hsa-miR-564”.

The term “hsa-miR-4763-3p gene” or “hsa-miR-4763-3p” used herein includes the hsa-miR-4763-3p gene (miRBase Accession No. MIMAT0019913) described in SEQ ID NO: 43, a homolog or an ortholog of a different organism species, and the like. The hsa-miR-4763-3p gene can be obtained by a method described in Persson H et al., 2011, Cancer Res, Vol. 71, p. 78-86. Also, “hsa-mir-4763” (miRBase Accession No. MI0017404, SEQ ID NO: 313) having a hairpin-like structure is known as a precursor of “hsa-miR-4763-3p”.

The term “hsa-miR-575 gene” or “hsa-miR-575” used herein includes the hsa-miR-575 gene (miRBase Accession No. MIMAT0003240) described in SEQ ID NO: 44, a homolog or an ortholog of a different organism species, and the like. The hsa-miR-575 gene can be obtained by a method described in Cummins J M et al., 2006, Proc Natl Acad Sci USA, Vol. 103, p. 3687-3692. Also, “hsa-mir-575” (miRBase Accession No. MI0003582, SEQ ID NO: 314) having a hairpin-like structure is known as a precursor of “hsa-miR-575”.

The term “hsa-miR-6771-5p gene” or “hsa-miR-6771-5p” used herein includes the hsa-miR-6771-5p gene (miRBase Accession No. MIMAT0027442) described in SEQ ID NO: 45, a homolog or an ortholog of a different organism species, and the like. The hsa-miR-6771-5p gene can be obtained by a method described in Ladewig E et al., 2012, Genome Res, Vol. 22, p. 1634-1645. Also, “hsa-mir-6771” (miRBase Accession No. MI0022616, SEQ ID NO: 315) having a hairpin-like structure is known as a precursor of “hsa-miR-6771-5p”.

The term “hsa-miR-1231 gene” or “hsa-miR-1231” used herein includes the hsa-miR-1231 gene (miRBase Accession No. MIMAT0005586) described in SEQ ID NO: 46, a homolog or an ortholog of a different organism species, and the like. The hsa-miR-1231 gene can be obtained by a method described in Berezikov E et al., 2007, Mol Cell, Vol. 28, p. 328-336. Also, “hsa-mir-1231” (miRBase Accession No. MI0006321, SEQ ID NO: 316) having a hairpin-like structure is known as a precursor of “hsa-miR-1231”.

The term “hsa-miR-1908-3p gene” or “hsa-miR-1908-3p” used herein includes the hsa-miR-1908-3p gene (miRBase Accession No. MIMAT0026916) described in SEQ ID NO: 47, a homolog or an ortholog of a different organism species, and the like. The hsa-miR-1908-3p gene can be obtained by a method described in Bar M et al., 2008, Stem Cells, Vol. 26, p. 2496-2505. Also, “hsa-mir-1908” (miRBase Accession No. MI0008329, SEQ ID NO: 317) having a hairpin-like structure is known as a precursor of “hsa-miR-1908-3p”.

The term “hsa-miR-150-3p gene” or “hsa-miR-150-3p” used herein includes the hsa-miR-150-3p gene (miRBase Accession No. MIMAT0004610) described in SEQ ID NO: 48, a homolog or an ortholog of a different organism species, and the like. The hsa-miR-150-3p gene can be obtained by a method described in Lagos-Quintana M et al., 2002, Curr Biol, Vol. 12, p. 735-739. Also, “hsa-mir-150” (miRBase Accession No. MI0000479, SEQ ID NO: 318) having a hairpin-like structure is known as a precursor of “hsa-miR-150-3p”.

The term “hsa-miR-3937 gene” or “hsa-miR-3937” used herein includes the hsa-miR-3937 gene (miRBase Accession No. MIMAT0018352) described in SEQ ID NO: 49, a homolog or an ortholog of a different organism species, and the like. The hsa-miR-3937 gene can be obtained by a method described in Liao J Y et al., 2010, PLoS One, Vol. 5, e10563. Also, “hsa-mir-3937” (miRBase Accession No. MI0016593, SEQ ID NO: 319) having a hairpin-like structure is known as a precursor of “hsa-miR-3937”.

The term “hsa-miR-887-3p gene” or “hsa-miR-887-3p” used herein includes the hsa-miR-887-3p gene (miRBase Accession No. MIMAT0004951) described in SEQ ID NO: 50, a homolog or an ortholog of a different organism species, and the like. The hsa-miR-887-3p gene can be obtained by a method described in Berezikov E et al., 2006, Genome Res, Vol. 16, p. 1289-1298. Also, “hsa-mir-887” (miRBase Accession No. MI0005562, SEQ ID NO: 320) having a hairpin-like structure is known as a precursor of “hsa-miR-887-3p”.

The term “hsa-miR-3940-5p gene” or “hsa-miR-3940-5p” used herein includes the hsa-miR-3940-5p gene (miRBase Accession No. MIMAT0019229) described in SEQ ID NO: 51, a homolog or an ortholog of a different organism species, and the like. The hsa-miR-3940-5p gene can be obtained by a method described in Liao J Y et al., 2010, PLoS One, Vol. 5, e10563. Also, “hsa-mir-3940” (miRBase Accession No. MI0016597, SEQ ID NO: 321) having a hairpin-like structure is known as a precursor of “hsa-miR-3940-5p”.

The term “hsa-miR-4741 gene” or “hsa-miR-4741” used herein includes the hsa-miR-4741 gene (miRBase Accession No. MIMAT0019871) described in SEQ ID NO: 52, a homolog or an ortholog of a different organism species, and the like. The hsa-miR-4741 gene can be obtained by a method described in Persson H et al., 2011, Cancer Res, Vol. 71, p. 78-86. Also, “hsa-mir-4741” (miRBase Accession No. MI0017379, SEQ ID NO: 322) having a hairpin-like structure is known as a precursor of “hsa-miR-4741”.

The term “hsa-miR-6808-5p gene” or “hsa-miR-6808-5p” used herein includes the hsa-miR-6808-5p gene (miRBase Accession No. MIMAT0027516) described in SEQ ID NO: 53, a homolog or an ortholog of a different organism species, and the like. The hsa-miR-6808-5p gene can be obtained by a method described in Ladewig E et al., 2012, Genome Res, Vol. 22, p. 1634-1645. Also, “hsa-mir-6808” (miRBase Accession No. MI0022653, SEQ ID NO: 323) having a hairpin-like structure is known as a precursor of “hsa-miR-6808-5p”.

The term “hsa-miR-6869-5p gene” or “hsa-miR-6869-5p” used herein includes the hsa-miR-6869-5p gene (miRBase Accession No. MIMAT0027638) described in SEQ ID NO: 54, a homolog or an ortholog of a different organism species, and the like. The hsa-miR-6869-5p gene can be obtained by a method described in Ladewig E et al., 2012, Genome Res, Vol. 22, p. 1634-1645. Also, “hsa-mir-6869” (miRBase Accession No. MI0022716, SEQ ID NO: 324) having a hairpin-like structure is known as a precursor of “hsa-miR-6869-5p”.

The term “hsa-miR-5090 gene” or “hsa-miR-5090” used herein includes the hsa-miR-5090 gene (miRBase Accession No. MIMAT0021082) described in SEQ ID NO: 55, a homolog or an ortholog of a different organism species, and the like. The hsa-miR-5090 gene can be obtained by a method described in Ding N et al., 2011, J Radiat Res, Vol. 52, p. 425-432. Also, “hsa-mir-5090” (miRBase Accession No. MI0017979, SEQ ID NO: 325) having a hairpin-like structure is known as a precursor of “hsa-miR-5090”.

The term “hsa-miR-615-5p gene” or “hsa-miR-615-5p” used herein includes the hsa-miR-615-5p gene (miRBase Accession No. MIMAT0004804) described in SEQ ID NO: 56, a homolog or an ortholog of a different organism species, and the like. The hsa-miR-615-5p gene can be obtained by a method described in Cummins J M et al., 2006, Proc Natl Acad Sci USA, Vol. 103, p. 3687-3692. Also, “hsa-mir-615” (miRBase Accession No. MI0003628, SEQ ID NO: 326) having a hairpin-like structure is known as a precursor of “hsa-miR-615-5p”.

The term “hsa-miR-8072 gene” or “hsa-miR-8072” used herein includes the hsa-miR-8072 gene (miRBase Accession No. MIMAT0030999) described in SEQ ID NO: 57, a homolog or an ortholog of a different organism species, and the like. The hsa-miR-8072 gene can be obtained by a method described in Wang H J et al., 2013, Shock, Vol. 39, p. 480-487. Also, “hsa-mir-8072” (miRBase Accession No. MI0025908, SEQ ID NO: 327) having a hairpin-like structure is known as a precursor of “hsa-miR-8072”.

The term “hsa-miR-128-1-5p gene” or “hsa-miR-128-1-5p” used herein includes the hsa-miR-128-1-5p gene (miRBase Accession No. MIMAT0026477) described in SEQ ID NO: 58, a homolog or an ortholog of a different organism species, and the like. The hsa-miR-128-1-5p gene can be obtained by a method described in Lagos-Quintana M et al., 2002, Curr Biol, Vol. 12, p. 735-739. Also, “hsa-mir-128-1” (miRBase Accession No. MI0000447, SEQ ID NO: 328) having a hairpin-like structure is known as a precursor of “hsa-miR-128-1-5p”.

The term “hsa-miR-1238-5p gene” or “hsa-miR-1238-5p” used herein includes the hsa-miR-1238-5p gene (miRBase Accession No. MIMAT0022947) described in SEQ ID NO: 59, a homolog or an ortholog of a different organism species, and the like. The hsa-miR-1238-5p gene can be obtained by a method described in Berezikov E et al., 2007, Mol Cell, Vol. 28, p. 328-336. Also, “hsa-mir-1238” (miRBase Accession No. MI0006328, SEQ ID NO: 329) having a hairpin-like structure is known as a precursor of “hsa-miR-1238-5p”.

The term “hsa-miR-365a-5p gene” or “hsa-miR-365a-5p” used herein includes the hsa-miR-365a-5p gene (miRBase Accession No. MIMAT0009199) described in SEQ ID NO: 60, a homolog or an ortholog of a different organism species, and the like. The hsa-miR-365a-5p gene can be obtained by a method described in Xie X et al., 2005, Nature, Vol. 434, p. 338-345. Also, “hsa-mir-365a” (miRBase Accession No. MI0000767, SEQ ID NO: 330) having a hairpin-like structure is known as a precursor of “hsa-miR-365a-5p”.

The term “hsa-miR-204-3p gene” or “hsa-miR-204-3p” used herein includes the hsa-miR-204-3p gene (miRBase Accession No. MIMAT0022693) described in SEQ ID NO: 61, a homolog or an ortholog of a different organism species, and the like. The hsa-miR-204-3p gene can be obtained by a method described in Lim L P et al., 2003, Science, Vol. 299, p. 1540. Also, “hsa-mir-204” (miRBase Accession No. MI0000284, SEQ ID NO: 331) having a hairpin-like structure is known as a precursor of “hsa-miR-204-3p”.

The term “hsa-miR-4492 gene” or “hsa-miR-4492” used herein includes the hsa-miR-4492 gene (miRBase Accession No. MIMAT0019027) described in SEQ ID NO: 62, a homolog or an ortholog of a different organism species, and the like. The hsa-miR-4492 gene can be obtained by a method described in Jima D D et al., 2010, Blood, Vol. 116, e118-e127. Also, “hsa-mir-4492” (miRBase Accession No. MI0016854, SEQ ID NO: 332) having a hairpin-like structure is known as a precursor of “hsa-miR-4492”.

The term “hsa-miR-6785-5p gene” or “hsa-miR-6785-5p” used herein includes the hsa-miR-6785-5p gene (miRBase Accession No. MIMAT0027470) described in SEQ ID NO: 63, a homolog or an ortholog of a different organism species, and the like. The hsa-miR-6785-5p gene can be obtained by a method described in Ladewig E et al., 2012, Genome Res, Vol. 22, p. 1634-1645. Also, “hsa-mir-6785” (miRBase Accession No. MI0022630, SEQ ID NO: 333) having a hairpin-like structure is known as a precursor of “hsa-miR-6785-5p”.

The term “hsa-miR-6511a-5p gene” or “hsa-miR-6511a-5p” used herein includes the hsa-miR-6511a-5p gene (miRBase Accession No. MIMAT0025478) described in SEQ ID NO: 64, a homolog or an ortholog of a different organism species, and the like. The hsa-miR-6511a-5p gene can be obtained by a method described in Joyce C E et al., 2011, Hum Mol Genet, Vol. 20, p. 4025-4040. Also, “hsa-mir-6511a-1, hsa-mir-6511a-2, hsa-mir-6511a-3, and hsa-mir-6511a-4” (miRBase Accession Nos. MI0022223, MI0023564, MI0023565, and MI0023566, SEQ ID NOs: 334, 335, 336, and 337) having a hairpin-like structure are known as precursors of “hsa-miR-6511a-5p”.

The term “hsa-miR-4525 gene” or “hsa-miR-4525” used herein includes the hsa-miR-4525 gene (miRBase Accession No. MIMAT0019064) described in SEQ ID NO: 65, a homolog or an ortholog of a different organism species, and the like. The hsa-miR-4525 gene can be obtained by a method described in lima D D et al., 2010, Blood, Vol. 116, e118-e127. Also, “hsa-mir-4525” (miRBase Accession No. MI0016892, SEQ ID NO: 338) having a hairpin-like structure is known as a precursor of “hsa-miR-4525”.

The term “hsa-miR-1915-5p gene” or “hsa-miR-1915-5p” used herein includes the hsa-miR-1915-5p gene (miRBase Accession No. MIMAT0007891) described in SEQ ID NO: 66, a homolog or an ortholog of a different organism species, and the like. The hsa-miR-1915-5p gene can be obtained by a method described in Bar M et al., 2008, Stem Cells, Vol. 26, p. 2496-2505. Also, “hsa-mir-1915” (miRBase Accession No. MI0008336, SEQ ID NO: 339) having a hairpin-like structure is known as a precursor of “hsa-miR-1915-5p”.

The term “hsa-miR-3180 gene” or “hsa-miR-3180” used herein includes the hsa-miR-3180 gene (miRBase Accession No. MIMAT0018178) described in SEQ ID NO: 67, a homolog or an ortholog of a different organism species, and the like. The hsa-miR-3180 gene can be obtained by a method described in Creighton Cl et al., 2010, PLoS One, Vol. 5, e9637. Also, “hsa-mir-3180-4 and hsa-mir-3180-5” (miRBase Accession Nos. MI0016408 and MI0016409, SEQ ID NOs: 340 and 341) having a hairpin-like structure are known as precursors of “hsa-miR-3180”.

The term “hsa-miR-6879-5p gene” or “hsa-miR-6879-5p” used herein includes the hsa-miR-6879-5p gene (miRBase Accession No. MIMAT0027658) described in SEQ ID NO: 68, a homolog or an ortholog of a different organism species, and the like. The hsa-miR-6879-5p gene can be obtained by a method described in Ladewig E et al., 2012, Genome Res, Vol. 22, p. 1634-1645. Also, “hsa-mir-6879” (miRBase Accession No. MI0022726, SEQ ID NO: 342) having a hairpin-like structure is known as a precursor of “hsa-miR-6879-5p”.

The term “hsa-miR-1199-5p gene” or “hsa-miR-1199-5p” used herein includes the hsa-miR-1199-5p gene (miRBase Accession No. MIMAT0031119) described in SEQ ID NO: 69, a homolog or an ortholog of a different organism species, and the like. The hsa-miR-1199-5p gene can be obtained by a method described in Salvi A et al., 2013, Int J Oncol, Vol. 42, p. 391-402. Also, “hsa-mir-1199” (miRBase Accession No. MI0020340,SEQ ID NO: 343) having a hairpin-like structure is known as a precursor of “hsa-miR-1199-5p”.

The term “hsa-miR-6746-5p gene” or “hsa-miR-6746-5p” used herein includes the hsa-miR-6746-5p gene (miRBase Accession No. MIMAT0027392) described in SEQ ID NO: 70, a homolog or an ortholog of a different organism species, and the like. The hsa-miR-6746-5p gene can be obtained by a method described in Ladewig E et al., 2012, Genome Res, Vol. 22, p. 1634-1645. Also, “hsa-mir-6746” (miRBase Accession No. MI0022591, SEQ ID NO: 344) having a hairpin-like structure is known as a precursor of “hsa-miR-6746-5p”.

The term “hsa-miR-711 gene” or “hsa-miR-711” used herein includes the hsa-miR-711 gene (miRBase Accession No. MIMAT0012734) described in SEQ ID NO: 71, a homolog or an ortholog of a different organism species, and the like. The hsa-miR-711 gene can be obtained by a method described in Artzi S et al., 2008, BMC Bioinformatics, Vol. 9, p. 39. Also, “hsa-mir-711” (miRBase Accession No. MI0012488, SEQ ID NO: 345) having a hairpin-like structure is known as a precursor of “hsa-miR-711”.

The term “hsa-miR-663b gene” or “hsa-miR-663b” used herein includes the hsa-miR-663b gene (miRBase Accession No. MIMAT0005867) described in SEQ ID NO: 72, a homolog or an ortholog of a different organism species, and the like. The hsa-miR-663b gene can be obtained by a method described in Takada S et al., 2008, Leukemia, Vol. 22, p. 1274-1278. Also, “hsa-mir-663b” (miRBase Accession No. MI0006336, SEQ ID NO: 346) having a hairpin-like structure is known as a precursor of “hsa-miR-663b”.

The term “hsa-miR-4707-3p gene” or “hsa-miR-4707-3p” used herein includes the hsa-miR-4707-3p gene (miRBase Accession No. MIMAT0019808) described in SEQ ID NO: 73, a homolog or an ortholog of a different organism species, and the like. The hsa-miR-4707-3p gene can be obtained by a method described in Persson H et al., 2011, Cancer Res, Vol. 71, p. 78-86. Also, “hsa-mir-4707” (miRBase Accession No. MI0017340, SEQ ID NO: 347) having a hairpin-like structure is known as a precursor of “hsa-miR-4707-3p”.

The term “hsa-miR-6893-5p gene” or “hsa-miR-6893-5p” used herein includes the hsa-miR-6893-5p gene (miRBase Accession No. MIMAT0027686) described in SEQ ID NO: 74, a homolog or an ortholog of a different organism species, and the like. The hsa-miR-6893-5p gene can be obtained by a method described in Ladewig E et al., 2012, Genome Res, Vol. 22, p. 1634-1645. Also, “hsa-mir-6893” (miRBase Accession No. MI0022740, SEQ ID NO: 348) having a hairpin-like structure is known as a precursor of “hsa-miR-6893-5p”.

The term “hsa-miR-4675 gene” or “hsa-miR-4675” used herein includes the hsa-miR-4675 gene (miRBase Accession No. MIMAT0019757) described in SEQ ID NO: 75, a homolog or an ortholog of a different organism species, and the like. The hsa-miR-4675 gene can be obtained by a method described in Persson H et al., 2011, Cancer Res, Vol. 71, p. 78-86. Also, “hsa-mir-4675” (miRBase Accession No. MI0017306, SEQ ID NO: 349) having a hairpin-like structure is known as a precursor of “hsa-miR-4675”.

The term “hsa-miR-4638-5p gene” or “hsa-miR-4638-5p” used herein includes the hsa-miR-4638-5p gene (miRBase Accession No. MIMAT0019695) described in SEQ ID NO: 76, a homolog or an ortholog of a different organism species, and the like. The hsa-miR-4638-5p gene can be obtained by a method described in Persson H et al., 2011, Cancer Res, Vol. 71, p. 78-86. Also, “hsa-mir-4638” (miRBase Accession No. MI0017265, SEQ ID NO: 350) having a hairpin-like structure is known as a precursor of “hsa-miR-4638-5p”.

The term “hsa-miR-4651 gene” or “hsa-miR-4651” used herein includes the hsa-miR-4651 gene (miRBase Accession No. MIMAT0019715) described in SEQ ID NO: 77, a homolog or an ortholog of a different organism species, and the like. The hsa-miR-4651 gene can be obtained by a method described in Persson H et al., 2011, Cancer Res, Vol. 71, p. 78-86. Also, “hsa-mir-4651” (miRBase Accession No. MI0017279, SEQ ID NO: 351) having a hairpin-like structure is known as a precursor of “hsa-miR-4651”.

The term “hsa-miR-6087 gene” or “hsa-miR-6087” used herein includes the hsa-miR-6087 gene (miRBase Accession No. MIMAT0023712) described in SEQ ID NO: 78, a homolog or an ortholog of a different organism species, and the like. The hsa-miR-6087 gene can be obtained by a method described in Yoo J K et al., 2012, Stem Cells Dev, Vol. 21, p. 2049-2057. Also, “hsa-mir-6087” (miRBase Accession No. MI0020364, SEQ ID NO: 352) having a hairpin-like structure is known as a precursor of “hsa-miR-6087”.

The term “hsa-miR-4665-5p gene” or “hsa-miR-4665-5p” used herein includes the hsa-miR-4665-5p gene (miRBase Accession No. MIMAT0019739) described in SEQ ID NO: 79, a homolog or an ortholog of a different organism species, and the like. The hsa-miR-4665-5p gene can be obtained by a method described in Persson H et al., 2011, Cancer Res, Vol. 71, p. 78-86. Also, “hsa-mir-4665” (miRBase Accession No. MI0017295, SEQ ID NO: 353) having a hairpin-like structure is known as a precursor of “hsa-miR-4665-5p”.

The term “hsa-miR-4758-5p gene” or “hsa-miR-4758-5p” used herein includes the hsa-miR-4758-5p gene (miRBase Accession No. MIMAT0019903) described in SEQ ID NO: 80, a homolog or an ortholog of a different organism species, and the like. The hsa-miR-4758-5p gene can be obtained by a method described in Persson H et al., 2011, Cancer Res, Vol. 71, p. 78-86. Also, “hsa-mir-4758” (miRBase Accession No. MI0017399, SEQ ID NO: 354) having a hairpin-like structure is known as a precursor of “hsa-miR-4758-5p”.

The term “hsa-miR-6887-5p gene” or “hsa-miR-6887-5p” used herein includes the hsa-miR-6887-5p gene (miRBase Accession No. MIMAT0027674) described in SEQ ID NO: 81, a homolog or an ortholog of a different organism species, and the like. The hsa-miR-6887-5p gene can be obtained by a method described in Ladewig E et al., 2012, Genome Res, Vol. 22, p. 1634-1645. Also, “hsa-mir-6887” (miRBase Accession No. MI0022734, SEQ ID NO: 355) having a hairpin-like structure is known as a precursor of “hsa-miR-6887-5p”.

The term “hsa-miR-3620-5p gene” or “hsa-miR-3620-5p” used herein includes the hsa-miR-3620-5p gene (miRBase Accession No. MIMAT0022967) described in SEQ ID NO: 82, a homolog or an ortholog of a different organism species, and the like. The hsa-miR-3620-5p gene can be obtained by a method described in Witten D et al., 2010, BMC Biol, Vol. 8, p. 58. Also, “hsa-mir-3620” (miRBase Accession No. MI0016011, SEQ ID NO: 356) having a hairpin-like structure is known as a precursor of “hsa-miR-3620-5p”.

The term “hsa-miR-1909-3p gene” or “hsa-miR-1909-3p” used herein includes the hsa-miR-1909-3p gene (miRBase Accession No. MIMAT0007883) described in SEQ ID NO: 83, a homolog or an ortholog of a different organism species, and the like. The hsa-miR-1909-3p gene can be obtained by a method described in Bar M et al., 2008, Stem Cells, Vol. 26, p. 2496-2505. Also, “hsa-mir-1909” (miRBase Accession No. MI0008330, SEQ ID NO: 357) having a hairpin-like structure is known as a precursor of “hsa-miR-1909-3p”.

The term “hsa-miR-7641 gene” or “hsa-miR-7641” used herein includes the hsa-miR-7641 gene (miRBase Accession No. MIMAT0029782) described in SEQ ID NO: 84, a homolog or an ortholog of a different organism species, and the like. The hsa-miR-7641 gene can be obtained by a method described in Yoo J K et al., 2013, Arch Pharm Res, Vol. 36, p. 353-358. Also, “hsa-mir-7641-1 and hsa-mir-7641-2” (miRBase Accession Nos. MI0024975 and MI0024976, SEQ ID NOs: 358 and 359) having a hairpin-like structure are known as precursors of “hsa-miR-7641”.

The term “hsa-miR-6724-5p gene” or “hsa-miR-6724-5p” used herein includes the hsa-miR-6724-5p gene (miRBase Accession No. MIMAT0025856) described in SEQ ID NO: 85, a homolog or an ortholog of a different organism species, and the like. The hsa-miR-6724-5p gene can be obtained by a method described in Li Y et al., 2012, Gene, Vol. 497, p. 330-335. Also, “hsa-mir-6724” (miRBase Accession No. MI0022559, SEQ ID NO: 360) having a hairpin-like structure is known as a precursor of “hsa-miR-6724-5p”.

The term “hsa-miR-1343-3p gene” or “hsa-miR-1343-3p” used herein includes the hsa-miR-1343-3p gene (miRBase Accession No. MIMAT0019776) described in SEQ ID NO: 86, a homolog or an ortholog of a different organism species, and the like. The hsa-miR-1343-3p gene can be obtained by a method described in Persson H et al., 2011, Cancer Res, Vol. 71, p. 78-86. Also, “hsa-mir-1343” (miRBase Accession No. MI0017320, SEQ ID NO: 361) having a hairpin-like structure is known as a precursor of “hsa-miR-1343-3p”.

The term “hsa-miR-6780b-5p gene” or “hsa-miR-6780b-5p” used herein includes the hsa-miR-6780b-5p gene (miRBase Accession No. MIMAT0027572) described in SEQ ID NO: 87, a homolog or an ortholog of a different organism species, and the like. The hsa-miR-6780b-5p gene can be obtained by a method described in Ladewig E et al., 2012, Genome Res, Vol. 22, p. 1634-1645. Also, “hsa-mir-6780b” (miRBase Accession No. MI0022681, SEQ ID NO: 362) having a hairpin-like structure is known as a precursor of “hsa-miR-6780b-5p”.

The term “hsa-miR-4484 gene” or “hsa-miR-4484” used herein includes the hsa-miR-4484 gene (miRBase Accession No. MIMAT0019018) described in SEQ ID NO: 88, a homolog or an ortholog of a different organism species, and the like. The hsa-miR-4484 gene can be obtained by a method described in Jima D D et al., 2010, Blood, Vol. 116, e118-e127. Also, “hsa-mir-4484” (miRBase Accession No. MI0016845, SEQ ID NO: 363) having a hairpin-like structure is known as a precursor of “hsa-miR-4484”.

The term “hsa-miR-4690-5p gene” or “hsa-miR-4690-5p” used herein includes the hsa-miR-4690-5p gene (miRBase Accession No. MIMAT0019779) described in SEQ ID NO: 89, a homolog or an ortholog of a different organism species, and the like. The hsa-miR-4690-5p gene can be obtained by a method described in Persson H et al., 2011, Cancer Res, Vol. 71, p. 78-86. Also, “hsa-mir-4690” (miRBase Accession No. MI0017323, SEQ ID NO: 364) having a hairpin-like structure is known as a precursor of “hsa-miR-4690-5p”.

The term “hsa-miR4429 gene” or “hsa-miR-4429” used herein includes the hsa-miR-4429 gene (miRBase Accession No. MIMAT0018944) described in SEQ ID NO: 90, a homolog or an ortholog of a different organism species, and the like. The hsa-miR-4429 gene can be obtained by a method described in Jima D D et al., 2010, Blood, Vol. 116, e118-e127. Also, “hsa-mir-4429” (miRBase Accession No. MI0016768, SEQ ID NO: 365) having a hairpin-like structure is known as a precursor of “hsa-miR4429”.

The term “hsa-miR-1227-5p gene” or “hsa-miR-1227-5p” used herein includes the hsa-miR-1227-5p gene (miRBase Accession No. MIMAT0022941) described in SEQ ID NO: 91, a homolog or an ortholog of a different organism species, and the like. The hsa-miR-1227-5p gene can be obtained by a method described in Berezikov E et al., 2007, Mol Cell, Vol. 28, p. 328-336. Also, “hsa-mir-1227” (miRBase Accession No. MI0006316, SEQ ID NO: 366) having a hairpin-like structure is known as a precursor of “hsa-miR-1227-5p”.

The term “hsa-miR-4725-3p gene” or “hsa-miR-4725-3p” used herein includes the hsa-miR-4725-3p gene (miRBase Accession No. MIMAT0019844) described in SEQ ID NO: 92, a homolog or an ortholog of a different organism species, and the like. The hsa-miR-4725-3p gene can be obtained by a method described in Persson H et al., 2011, Cancer Res, Vol. 71, p. 78-86. Also, “hsa-mir-4725” (miRBase Accession No. MI0017362, SEQ ID NO: 367) having a hairpin-like structure is known as a precursor of “hsa-miR-4725-3p”.

The term “hsa-miR-6861-5p gene” or “hsa-miR-6861-5p” used herein includes the hsa-miR-6861-5p gene (miRBase Accession No. MIMAT0027623) described in SEQ ID NO: 93, a homolog or an ortholog of a different organism species, and the like. The hsa-miR-6861-5p gene can be obtained by a method described in Ladewig E et al., 2012, Genome Res, Vol. 22, p. 1634-1645. Also, “hsa-mir-6861” (miRBase Accession No. MI0022708, SEQ ID NO: 368) having a hairpin-like structure is known as a precursor of “hsa-miR-6861-5p”.

The term “hsa-miR-6812-5p gene” or “hsa-miR-6812-5p” used herein includes the hsa-miR-6812-5p gene (miRBase Accession No. MIMAT0027524) described in SEQ ID NO: 94, a homolog or an ortholog of a different organism species, and the like. The hsa-miR-6812-5p gene can be obtained by a method described in Ladewig E et al., 2012, Genome Res, Vol. 22, p. 1634-1645. Also, “hsa-mir-6812” (miRBase Accession No. MI0022657, SEQ ID NO: 369) having a hairpin-like structure is known as a precursor of “hsa-miR-6812-5p”.

The term “hsa-miR-3197 gene” or “hsa-miR-3197” used herein includes the hsa-miR-3197 gene (miRBase Accession No. MIMAT0015082) described in SEQ ID NO: 95, a homolog or an ortholog of a different organism species, and the like. The hsa-miR-3197 gene can be obtained by a method described in Stark M S et al., 2010, PLoS One, Vol. 5, e9685. Also, “hsa-mir-3197” (miRBase Accession No. MI0014245, SEQ ID NO: 370) having a hairpin-like structure is known as a precursor of “hsa-miR-3197”.

The term “hsa-miR-8059 gene” or “hsa-miR-8059” used herein includes the hsa-miR-8059 gene (miRBase Accession No. MIMAT0030986) described in SEQ ID NO: 96, a homolog or an ortholog of a different organism species, and the like. The hsa-miR-8059 gene can be obtained by a method described in Wang H J et al., 2013, Shock, Vol. 39, p. 480-487. Also, “hsa-mir-8059” (miRBase Accession No. MI0025895, SEQ ID NO: 371) having a hairpin-like structure is known as a precursor of “hsa-miR-8059”.

The term “hsa-miR-3185 gene” or “hsa-miR-3185” used herein includes the hsa-miR-3185 gene (miRBase Accession No. MIMAT0015065) described in SEQ ID NO: 97, a homolog or an ortholog of a different organism species, and the like. The hsa-miR-3185 gene can be obtained by a method described in Stark M S et al., 2010, PLoS One, Vol. 5, e9685. Also, “hsa-mir-3185” (miRBase Accession No. MI0014227, SEQ ID NO: 372) having a hairpin-like structure is known as a precursor of “hsa-miR-3185”.

The term “hsa-miR-4706 gene” or “hsa-miR-4706” used herein includes the hsa-miR-4706 gene (miRBase Accession No. MIMAT0019806) described in SEQ ID NO: 98, a homolog or an ortholog of a different organism species, and the like. The hsa-miR-4706 gene can be obtained by a method described in Persson H et al., 2011, Cancer Res, Vol. 71, p. 78-86. Also, “hsa-mir-4706” (miRBase Accession No. MI0017339, SEQ ID NO: 373) having a hairpin-like structure is known as a precursor of “hsa-miR-4706”.

The term “hsa-miR-4497 gene” or “hsa-miR-4497” used herein includes the hsa-miR-4497 gene (miRBase Accession No. MIMAT0019032) described in SEQ ID NO: 99, a homolog or an ortholog of a different organism species, and the like. The hsa-miR-4497 gene can be obtained by a method described in Jima D D et al., 2010, Blood, Vol. 116, e118-e127. Also, “hsa-mir-4497” (miRBase Accession No. MI0016859, SEQ ID NO: 374) having a hairpin-like structure is known as a precursor of “hsa-miR-4497”.

The term “hsa-miR-3131 gene” or “hsa-miR-3131” used herein includes the hsa-miR-3131 gene (miRBase Accession No. MIMAT0014996) described in SEQ ID NO: 100, a homolog or an ortholog of a different organism species, and the like. The hsa-miR-3131 gene can be obtained by a method described in Stark M S et al., 2010, PLoS One, Vol. 5, e9685. Also, “hsa-mir-3131” (miRBase Accession No. MI0014151, SEQ ID NO: 375) having a hairpin-like structure is known as a precursor of “hsa-miR-3131”.

The term “hsa-miR-6806-5p gene” or “hsa-miR-6806-5p” used herein includes the hsa-miR-6806-5p gene (miRBase Accession No. MIMAT0027512) described in SEQ ID NO: 101, a homolog or an ortholog of a different organism species, and the like. The hsa-miR-6806-5p gene can be obtained by a method described in Ladewig E et al., 2012, Genome Res, Vol. 22, p. 1634-1645. Also, “hsa-mir-6806” (miRBase Accession No. MI0022651, SEQ ID NO: 376) having a hairpin-like structure is known as a precursor of “hsa-miR-6806-5p”.

The term “hsa-miR-187-5p gene” or “hsa-miR-187-5p” used herein includes the hsa-miR-187-5p gene (miRBase Accession No. MIMAT0004561) described in SEQ ID NO: 102, a homolog or an ortholog of a different organism species, and the like. The hsa-miR-187-5p gene can be obtained by a method described in Lim L P et al., 2003, Science, Vol. 299, p. 1540. Also, “hsa-mir-187” (miRBase Accession No. MI0000274, SEQ ID NO: 377) having a hairpin-like structure is known as a precursor of “hsa-miR-187-5p”.

The term “hsa-miR-3180-3p gene” or “hsa-miR-3180-3p” used herein includes the hsa-miR-3180-3p gene (miRBase Accession No. MIMAT0015058) described in SEQ ID NO: 103, a homolog or an ortholog of a different organism species, and the like. The hsa-miR-3180-3p gene can be obtained by a method described in Creighton C J et al., 2010, PLoS One, Vol. 5, e9637. Also, “hsa-mir-3180-1, hsa-mir-3180-2, and hsa-mir-3180-3” (miRBase Accession Nos. MI0014214, MI0014215, and MI0014217, SEQ ID NOs: 378, 379, and 380) having a hairpin-like structure are known as precursors of “hsa-miR-3180-3p”.

The term “hsa-miR-6848-5p gene” or “hsa-miR-6848-5p” used herein includes the hsa-miR-6848-5p gene (miRBase Accession No. MIMAT0027596) described in SEQ ID NO: 104, a homolog or an ortholog of a different organism species, and the like. The hsa-miR-6848-5p gene can be obtained by a method described in Ladewig E et al., 2012, Genome Res, Vol. 22, p. 1634-1645. Also, “hsa-mir-6848” (miRBase Accession No. MI0022694, SEQ ID NO: 381) having a hairpin-like structure is known as a precursor of “hsa-miR-6848-5p”.

The term “hsa-miR-6820-5p gene” or “hsa-miR-6820-5p” used herein includes the hsa-miR-6820-5p gene (miRBase Accession No. MIMAT0027540) described in SEQ ID NO: 105, a homolog or an ortholog of a different organism species, and the like. The hsa-miR-6820-5p gene can be obtained by a method described in Ladewig E et al., 2012, Genome Res, Vol. 22, p. 1634-1645. Also, “hsa-mir-6820” (miRBase Accession No. MI0022665, SEQ ID NO: 382) having a hairpin-like structure is known as a precursor of “hsa-miR-6820-5p”.

The term “hsa-miR-6800-5p gene” or “hsa-miR-6800-5p” used herein includes the hsa-miR-6800-5p gene (miRBase Accession No. MIMAT0027500) described in SEQ ID NO: 106, a homolog or an ortholog of a different organism species, and the like. The hsa-miR-6800-5p gene can be obtained by a method described in Ladewig E et al., 2012, Genome Res, Vol. 22, p. 1634-1645. Also, “hsa-mir-6800” (miRBase Accession No. MI0022645, SEQ ID NO: 383) having a hairpin-like structure is known as a precursor of “hsa-miR-6800-5p”.

The term “hsa-miR-6717-5p gene” or “hsa-miR-6717-5p” used herein includes the hsa-miR-6717-5p gene (miRBase Accession No. MIMAT0025846) described in SEQ ID NO: 107, a homolog or an ortholog of a different organism species, and the like. The hsa-miR-6717-5p gene can be obtained by a method described in Li Y et al., 2012, Gene, Vol. 497, p. 330-335. Also, “hsa-mir-6717” (miRBase Accession No. MI0022551, SEQ ID NO: 384) having a hairpin-like structure is known as a precursor of “hsa-miR-6717-5p”.

The term “hsa-miR-6795-5p gene” or “hsa-miR-6795-5p” used herein includes the hsa-miR-6795-5p gene (miRBase Accession No. MIMAT0027490) described in SEQ ID NO: 108, a homolog or an ortholog of a different organism species, and the like. The hsa-miR-6795-5p gene can be obtained by a method described in Ladewig E et al., 2012, Genome Res, Vol. 22, p. 1634-1645. Also, “hsa-mir-6795” (miRBase Accession No. MI0022640, SEQ ID NO: 385) having a hairpin-like structure is known as a precursor of “hsa-miR-6795-5p”.

The term “hsa-miR-4632-5p gene” or “hsa-miR-4632-5p” used herein includes the hsa-miR-4632-5p gene (miRBase Accession No. MIMAT0022977) described in SEQ ID NO: 109, a homolog or an ortholog of a different organism species, and the like. The hsa-miR-4632-5p gene can be obtained by a method described in Persson H et al., 2011, Cancer Res, Vol. 71, p. 78-86. Also, “hsa-mir-4632” (miRBase Accession No. MI0017259, SEQ ID NO: 386) having a hairpin-like structure is known as a precursor of “hsa-miR-4632-5p”.

The term “hsa-miR-665 gene” or “hsa-miR-665” used herein includes the hsa-miR-665 gene (miRBase Accession No. MIMAT0004952) described in SEQ ID NO: 110, a homolog or an ortholog of a different organism species, and the like. The hsa-miR-665 gene can be obtained by a method described in Berezikov E et al., 2006, Genome Res, Vol. 16, p. 1289-1298. Also, “hsa-mir-665” (miRBase Accession No. MI0005563, SEQ ID NO: 387) having a hairpin-like structure is known as a precursor of “hsa-miR-665”.

The term “hsa-miR-6778-5p gene” or “hsa-miR-6778-5p” used herein includes the hsa-miR-6778-5p gene (miRBase Accession No. MIMAT0027456) described in SEQ ID NO: 111, a homolog or an ortholog of a different organism species, and the like. The hsa-miR-6778-5p gene can be obtained by a method described in Ladewig E et al., 2012, Genome Res, Vol. 22, p. 1634-1645. Also, “hsa-mir-6778” (miRBase Accession No. MI0022623, SEQ ID NO: 388) having a hairpin-like structure is known as a precursor of “hsa-miR-6778-5p”.

The term “hsa-miR-3663-3p gene” or “hsa-miR-3663-3p” used herein includes the hsa-miR-3663-3p gene (miRBase Accession No. MIMAT0018085) described in SEQ ID NO: 112, a homolog or an ortholog of a different organism species, and the like. The hsa-miR-3663-3p gene can be obtained by a method described in Liao J Y et al., 2010, PLoS One, Vol. 5, e10563. Also, “hsa-mir-3663” (miRBase Accession No. MI0016064, SEQ ID NO: 389) having a hairpin-like structure is known as a precursor of “hsa-miR-3663-3p”.

The term “hsa-miR-4689 gene” or “hsa-miR-4689” used herein includes the hsa-miR-4689 gene (miRBase Accession No. MIMAT0019778) described in SEQ ID NO: 113, a homolog or an ortholog of a different organism species, and the like. The hsa-miR-4689 gene can be obtained by a method described in Persson H et al., 2011, Cancer Res, Vol. 71, p. 78-86. Also, “hsa-mir-4689” (miRBase Accession No. MI0017322, SEQ ID NO: 390) having a hairpin-like structure is known as a precursor of “hsa-miR-4689”.

The term “hsa-miR-211-3p gene” or “hsa-miR-211-3p” used herein includes the hsa-miR-211-3p gene (miRBase Accession No. MIMAT0022694) described in SEQ ID NO: 114, a homolog or an ortholog of a different organism species, and the like. The hsa-miR-211-3p gene can be obtained by a method described in Lim L P et al., 2003, Science, Vol. 299, p. 1540. Also, “hsa-mir-211” (miRBase Accession No. MI0000287, SEQ ID NO: 391) having a hairpin-like structure is known as a precursor of “hsa-miR-211-3p”.

The term “hsa-miR-6511b-5p gene” or “hsa-miR-6511b-5p” used herein includes the hsa-miR-6511b-5p gene (miRBase Accession No. MIMAT0025847) described in SEQ ID NO: 115, a homolog or an ortholog of a different organism species, and the like. The hsa-miR-6511b-5p gene can be obtained by a method described in Li Y et al., 2012, Gene, Vol. 497, p. 330-335. Also, “hsa-mir-6511b-1 and hsa-mir-6511b-2” (miRBase Accession Nos. MI0022552 and MI0023431, SEQ ID NOs: 392 and 393) having a hairpin-like structure are known as precursors of “hsa-miR-6511b-5p”.

The term “hsa-miR-4750-5p gene” or “hsa-miR-4750-5p” used herein includes the hsa-miR-4750-5p gene (miRBase Accession No. MIMAT0019887) described in SEQ ID NO: 116, a homolog or an ortholog of a different organism species, and the like. The hsa-miR-4750-5p gene can be obtained by a method described in Persson H et al., 2011, Cancer Res, Vol. 71, p. 78-86. Also, “hsa-mir-4750” (miRBase Accession No. MI0017389, SEQ ID NO: 394) having a hairpin-like structure is known as a precursor of “hsa-miR-4750-5p”.

The term “hsa-miR-6126 gene” or “hsa-miR-6126” used herein includes the hsa-miR-6126 gene (miRBase Accession No. MIMAT0024599) described in SEQ ID NO: 117, a homolog or an ortholog of a different organism species, and the like. The hsa-miR-6126 gene can be obtained by a method described in Smith J L et al., 2012, J Virol. Vol. 86, p. 5278-5287. Also, “hsa-mir-6126” (miRBase Accession No. MI0021260, SEQ ID NO: 395) having a hairpin-like structure is known as a precursor of “hsa-miR-6126”.

The term “hsa-miR-614 gene” or “hsa-miR-614” used herein includes the hsa-miR-614 gene (miRBase Accession No. MIMAT0003282) described in SEQ ID NO: 118, a homolog or an ortholog of a different organism species, and the like. The hsa-miR-614 gene can be obtained by a method described in Cummins J M et al., 2006, Proc Natl Acad Sci USA, Vol. 103, p. 3687-3692. Also, “hsa-mir-614” (miRBase Accession No. MI0003627, SEQ ID NO: 396) having a hairpin-like structure is known as a precursor of “hsa-miR-614”.

The term “hsa-miR-7110-5p gene” or “hsa-miR-7110-5p” used herein includes the hsa-miR-7110-5p gene (miRBase Accession No. MIMAT0028117) described in SEQ ID NO: 119, a homolog or an ortholog of a different organism species, and the like. The hsa-miR-7110-5p gene can be obtained by a method described in Ladewig E et al., 2012, Genome Res, Vol. 22, p. 1634-1645. Also, “hsa-mir-7110” (miRBase Accession No. MI0022961, SEQ ID NO: 397) having a hairpin-like structure is known as a precursor of “hsa-miR-7110-5p”.

The term “hsa-miR-744-5p gene” or “hsa-miR-744-5p” used herein includes the hsa-miR-744-5p gene (miRBase Accession No. MIMAT0004945) described in SEQ ID NO: 120, a homolog or an ortholog of a different organism species, and the like. The hsa-miR-744-5p gene can be obtained by a method described in Berezikov E et al., 2006, Genome Res, Vol. 16, p. 1289-1298. Also, “hsa-mir-744” (miRBase Accession No. MI0005559, SEQ ID NO: 398) having a hairpin-like structure is known as a precursor of “hsa-miR-744-5p”.

The term “hsa-miR-6769a-5p gene” or “hsa-miR-6769a-5p” used herein includes the hsa-miR-6769a-5p gene (miRBase Accession No. MIMAT0027438) described in SEQ ID NO: 121, a homolog or an ortholog of a different organism species, and the like. The hsa-miR-6769a-5p gene can be obtained by a method described in Ladewig E et al., 2012, Genome Res, Vol. 22, p. 1634-1645. Also, “hsa-mir-6769a” (miRBase Accession No. MI0022614, SEQ ID NO: 399) having a hairpin-like structure is known as a precursor of “hsa-miR-6769a-5p”.

The term “hsa-miR-4792 gene” or “hsa-miR-4792” used herein includes the hsa-miR-4792 gene (miRBase Accession No. MIMAT0019964) described in SEQ ID NO: 122, a homolog or an ortholog of a different organism species, and the like. The hsa-miR-4792 gene can be obtained by a method described in Persson H et al., 2011, Cancer Res, Vol. 71, p. 78-86. Also, “hsa-mir-4792” (miRBase Accession No. MI0017439, SEQ ID NO: 400) having a hairpin-like structure is known as a precursor of “hsa-miR-4792”.

The term “hsa-miR-5787 gene” or “hsa-miR-5787” used herein includes the hsa-miR-5787 gene (miRBase Accession No. MIMAT0023252) described in SEQ ID NO: 123, a homolog or an ortholog of a different organism species, and the like. The hsa-miR-5787 gene can be obtained by a method described in Yoo H et al., 2011, Biochem Biophys Res Commun, Vol. 415, p. 567-572. Also, “hsa-mir-5787” (miRBase Accession No. MI0019797, SEQ ID NO: 401) having a hairpin-like structure is known as a precursor of “hsa-miR-5787”.

The term “hsa-miR-6798-5p gene” or “hsa-miR-6798-5p” used herein includes the hsa-miR-6798-5p gene (miRBase Accession No. MIMAT0027496) described in SEQ ID NO: 124, a homolog or an ortholog of a different organism species, and the like. The hsa-miR-6798-5p gene can be obtained by a method described in Ladewig E et al., 2012, Genome Res, Vol. 22, p. 1634-1645. Also, “hsa-mir-6798” (miRBase Accession No. MI0022643, SEQ ID NO: 402) having a hairpin-like structure is known as a precursor of “hsa-miR-6798-5p”.

The term “hsa-miR-6781-5p gene” or “hsa-miR-6781-5p” used herein includes the hsa-miR-6781-5p gene (miRBase Accession No. MIMAT0027462) described in SEQ ID NO: 125, a homolog or an ortholog of a different organism species, and the like. The hsa-miR-6781-5p gene can be obtained by a method described in Ladewig E et al., 2012, Genome Res, Vol. 22, p. 1634-1645. Also, “hsa-mir-6781” (miRBase Accession No. MI0022626, SEQ ID NO: 403) having a hairpin-like structure is known as a precursor of “hsa-miR-6781-5p”.

The term “hsa-miR-4419b gene” or “hsa-miR-4419b” used herein includes the hsa-miR-4419b gene (miRBase Accession No. MIMAT0019034) described in SEQ ID NO: 126, a homolog or an ortholog of a different organism species, and the like. The hsa-miR-4419b gene can be obtained by a method described in Jima D D et al., 2010, Blood, Vol. 116, e118-e127. Also, “hsa-mir-4419b” (miRBase Accession No. MI0016861, SEQ ID NO: 404) having a hairpin-like structure is known as a precursor of “hsa-miR-4419b”.

The term “hsa-miR-4446-3p gene” or “hsa-miR-4446-3p” used herein includes the hsa-miR-4446-3p gene (miRBase Accession No. MIMAT0018965) described in SEQ ID NO: 127, a homolog or an ortholog of a different organism species, and the like. The hsa-miR-4446-3p gene can be obtained by a method described in Jima D D et al., 2010, Blood, Vol. 116, e118-e127. Also, “hsa-mir-4446” (miRBase Accession No. MI0016789, SEQ ID NO: 405) having a hairpin-like structure is known as a precursor of “hsa-miR-4446-3p”.

The term “hsa-miR-4259 gene” or “hsa-miR-4259” used herein includes the hsa-miR-4259 gene (miRBase Accession No. MIMAT0016880) described in SEQ ID NO: 128, a homolog or an ortholog of a different organism species, and the like. The hsa-miR-4259 gene can be obtained by a method described in Goff L A et al., 2009, PLoS One, Vol. 4, e7192. Also, “hsa-mir-4259” (miRBase Accession No. MI0015858, SEQ ID NO: 406) having a hairpin-like structure is known as a precursor of “hsa-miR-4259”.

The term “hsa-miR-5572 gene” or “hsa-miR-5572” used herein includes the hsa-miR-5572 gene (miRBase Accession No. MIMAT0022260) described in SEQ ID NO: 129, a homolog or an ortholog of a different organism species, and the like. The hsa-miR-5572 gene can be obtained by a method described in Tandon M et al., 2012, Oral Dis, Vol. 18, p. 127-131. Also, “hsa-mir-5572” (miRBase Accession No. MI0019117, SEQ ID NO: 407) having a hairpin-like structure is known as a precursor of “hsa-miR-5572”.

The term “hsa-miR-6075 gene” or “hsa-miR-6075” used herein includes the hsa-miR-6075 gene (miRBase Accession No. MIMAT0023700) described in SEQ ID NO: 130, a homolog or an ortholog of a different organism species, and the like. The hsa-miR-6075 gene can be obtained by a method described in Voellenkle C et al., 2012, RNA, Vol. 18, p. 472-484. Also, “hsa-mir-6075” (miRBase Accession No. MI0020352, SEQ ID NO: 408) having a hairpin-like structure is known as a precursor of “hsa-miR-6075”.

The term “hsa-miR-296-3p gene” or “hsa-miR-296-3p” used herein includes the hsa-miR-296-3p gene (miRBase Accession No. MIMAT0004679) described in SEQ ID NO: 131, a homolog or an ortholog of a different organism species, and the like. The hsa-miR-296-3p gene can be obtained by a method described in Houbaviy H B et al., 2003, Dev Cell, Vol. 5, p. 351-358. Also, “hsa-mir-296” (miRBase Accession No. MI0000747, SEQ ID NO: 409) having a hairpin-like structure is known as a precursor of “hsa-miR-296-3p”.

The term “hsa-miR-6891-5p gene” or “hsa-miR-6891-5p” used herein includes the hsa-miR-6891-5p gene (miRBase Accession No. MIMAT0027682) described in SEQ ID NO: 132, a homolog or an ortholog of a different organism species, and the like. The hsa-miR-6891-5p gene can be obtained by a method described in Ladewig E et al., 2012, Genome Res, Vol. 22, p. 1634-1645. Also, “hsa-mir-6891” (miRBase Accession No. MI0022738, SEQ ID NO: 410) having a hairpin-like structure is known as a precursor of “hsa-miR-6891-5p”.

The term “hsa-miR-4745-5p gene” or “hsa-miR-4745-5p” used herein includes the hsa-miR-4745-5p gene (miRBase Accession No. MIMAT0019878) described in SEQ ID NO: 133, a homolog or an ortholog of a different organism species, and the like. The hsa-miR-4745-5p gene can be obtained by a method described in Persson H et al., 2011, Cancer Res, Vol. 71, p. 78-86. Also, “hsa-mir-4745” (miRBase Accession No. MI0017384, SEQ ID NO: 411) having a hairpin-like structure is known as a precursor of “hsa-miR-4745-5p”.

The term “hsa-miR-6775-5p gene” or “hsa-miR-6775-5p” used herein includes the hsa-miR-6775-5p gene (miRBase Accession No. MIMAT0027450) described in SEQ ID NO: 134, a homolog or an ortholog of a different organism species, and the like. The hsa-miR-6775-5p gene can be obtained by a method described in Ladewig E et al., 2012, Genome Res, Vol. 22, p. 1634-1645. Also, “hsa-mir-6775” (miRBase Accession No. MI0022620, SEQ ID NO: 412) having a hairpin-like structure is known as a precursor of “hsa-miR-6775-5p”.

The term “hsa-miR-6870-5p gene” or “hsa-miR-6870-5p” used herein includes the hsa-miR-6870-5p gene (miRBase Accession No. MIMAT0027640) described in SEQ ID NO: 135, a homolog or an ortholog of a different organism species, and the like. The hsa-miR-6870-5p gene can be obtained by a method described in Ladewig E et al., 2012, Genome Res, Vol. 22, p. 1634-1645. Also, “hsa-mir-6870” (miRBase Accession No. MI0022717, SEQ ID NO: 413) having a hairpin-like structure is known as a precursor of “hsa-miR-6870-5p”.

The term “hsa-miR-920 gene” or “hsa-miR-920” used herein includes the hsa-miR-920 gene (miRBase Accession No. MIMAT0004970) described in SEQ ID NO: 136, a homolog or an ortholog of a different organism species, and the like. The hsa-miR-920 gene can be obtained by a method described in Novotny G W et al., 2007, Int J Androl, Vol. 30, p. 316-326. Also, “hsa-mir-920” (miRBase Accession No. MI0005712, SEQ ID NO: 414) having a hairpin-like structure is known as a precursor of “hsa-miR-920”.

The term “hsa-miR-4530 gene” or “hsa-miR-4530” used herein includes the hsa-miR-4530 gene (miRBase Accession No. MIMAT0019069) described in SEQ ID NO: 137, a homolog or an ortholog of a different organism species, and the like. The hsa-miR-4530 gene can be obtained by a method described in Jima D D et al., 2010, Blood, Vol. 116, e118-e127. Also, “hsa-mir-4530” (miRBase Accession No. MI0016897, SEQ ID NO: 415) having a hairpin-like structure is known as a precursor of “hsa-miR-4530”.

The term “hsa-miR-6819-5p gene” or “hsa-miR-6819-5p” used herein includes the hsa-miR-6819-5p gene (miRBase Accession No. MIMAT0027538) described in SEQ ID NO: 138, a homolog or an ortholog of a different organism species, and the like. The hsa-miR-6819-5p gene can be obtained by a method described in Ladewig E et al., 2012, Genome Res, Vol. 22, p. 1634-1645. Also, “hsa-mir-6819” (miRBase Accession No. MI0022664, SEQ ID NO: 416) having a hairpin-like structure is known as a precursor of “hsa-miR-6819-5p”.

The term “hsa-miR-6825-5p gene” or “hsa-miR-6825-5p” used herein includes the hsa-miR-6825-5p gene (miRBase Accession No. MIMAT0027550) described in SEQ ID NO: 139, a homolog or an ortholog of a different organism species, and the like. The hsa-miR-6825-5p gene can be obtained by a method described in Ladewig E et al., 2012, Genome Res, Vol. 22, p. 1634-1645. Also, “hsa-mir-6825” (miRBase Accession No. MI0022670, SEQ ID NO: 417) having a hairpin-like structure is known as a precursor of “hsa-miR-6825-5p”.

The term “hsa-miR-7847-3p gene” or “hsa-miR-7847-3p” used herein includes the hsa-miR-7847-3p gene (miRBase Accession No. MIMAT0030422) described in SEQ ID NO: 140, a homolog or an ortholog of a different organism species, and the like. The hsa-miR-7847-3p gene can be obtained by a method described in Pie H et al., 2012, PLoS One, Vol. 7, e50746. Also, “hsa-mir-7847” (miRBase Accession No. MI0025517, SEQ ID NO: 418) having a hairpin-like structure is known as a precursor of “hsa-miR-7847-3p”.

The term “hsa-miR-6131 gene” or “hsa-miR-6131” used herein includes the hsa-miR-6131 gene (miRBase Accession No. MIMAT0024615) described in SEQ ID NO: 141, a homolog or an ortholog of a different organism species, and the like. The hsa-miR-6131 gene can be obtained by a method described in Dannemann M et al., 2012, Genome Biol Evol, Vol. 4, p. 552-564. Also, “hsa-mir-6131” (miRBase Accession No. MI0021276, SEQ ID NO: 419) having a hairpin-like structure is known as a precursor of “hsa-miR-6131”.

The term “hsa-miR-4433-3p gene” or “hsa-miR-4433-3p” used herein includes the hsa-miR-4433-3p gene (miRBase Accession No. MIMAT0018949) described in SEQ ID NO: 142, a homolog or an ortholog of a different organism species, and the like. The hsa-miR-4433-3p gene can be obtained by a method described in Jima D D et al., 2010, Blood, Vol. 116, e118-e127. Also, “hsa-mir-4433” (miRBase Accession No. MI0016773, SEQ ID NO: 420) having a hairpin-like structure is known as a precursor of “hsa-miR-4433-3p”.

The term “hsa-miR-1228-5p gene” or “hsa-miR-1228-5p” used herein includes the hsa-miR-1228-5p gene (miRBase Accession No. MIMAT0005582) described in SEQ ID NO: 143, a homolog or an ortholog of a different organism species, and the like. The hsa-miR-1228-5p gene can be obtained by a method described in Berezikov E et al., 2007, Mol Cell, Vol. 28, p. 328-336. Also, “hsa-mir-1228” (miRBase Accession No. MI0006318, SEQ ID NO: 421) having a hairpin-like structure is known as a precursor of “hsa-miR-1228-5p”.

The term “hsa-miR-6743-5p gene” or “hsa-miR-6743-5p” used herein includes the hsa-miR-6743-5p gene (miRBase Accession No. MIMAT0027387) described in SEQ ID NO: 144, a homolog or an ortholog of a different organism species, and the like. The hsa-miR-6743-5p gene can be obtained by a method described in Ladewig E et al., 2012, Genome Res, Vol. 22, p. 1634-1645. Also, “hsa-mir-6743” (miRBase Accession No. MI0022588, SEQ ID NO: 422) having a hairpin-like structure is known as a precursor of “hsa-miR-6743-5p”.

The term “hsa-miR-1268a gene” or “hsa-miR-1268a” used herein includes the hsa-miR-1268a gene (miRBase Accession No. MIMAT0005922) described in SEQ ID NO: 145, a homolog or an ortholog of a different organism species, and the like. The hsa-miR-1268a gene can be obtained by a method described in Morin R D et al., 2008, Genome Res, Vol. 18, p. 610-621. Also, “hsa-mir-1268a” (miRBase Accession No. MI0006405, SEQ ID NO: 423) having a hairpin-like structure is known as a precursor of “hsa-miR-1268a”.

The term “hsa-miR-3917 gene” or “hsa-miR-3917” used herein includes the hsa-miR-3917 gene (miRBase Accession No. MIMAT0018191) described in SEQ ID NO: 146, a homolog or an ortholog of a different organism species, and the like. The hsa-miR-3917 gene can be obtained by a method described in Creighton C) et al., 2010, PLoS One, Vol. 5, e9637. Also, “hsa-mir-3917” (miRBase Accession No. MI0016423, SEQ ID NO: 424) having a hairpin-like structure is known as a precursor of “hsa-miR-3917”.

The term “hsa-miR-6786-5p gene” or “hsa-miR-6786-5p” used herein includes the hsa-miR-6786-5p gene (miRBase Accession No. MIMAT0027472) described in SEQ ID NO: 147, a homolog or an ortholog of a different organism species, and the like. The hsa-miR-6786-5p gene can be obtained by a method described in Ladewig E et al., 2012, Genome Res, Vol. 22, p. 1634-1645. Also, “hsa-mir-6786” (miRBase Accession No. MI0022631, SEQ ID NO: 425) having a hairpin-like structure is known as a precursor of “hsa-miR-6786-5p”.

The term “hsa-miR-3154 gene” or “hsa-miR-3154” used herein includes the hsa-miR-3154 gene (miRBase Accession No. MIMAT0015028) described in SEQ ID NO: 148, a homolog or an ortholog of a different organism species, and the like. The hsa-miR-3154 gene can be obtained by a method described in Berezikov E et al., 2006, Genome Res, Vol. 16, p. 1289-1298. Also, “hsa-mir-3154” (miRBase Accession No. MI0014182, SEQ ID NO: 426) having a hairpin-like structure is known as a precursor of “hsa-miR-3154”.

The term “hsa-miR-638 gene” or “hsa-miR-638” used herein includes the hsa-miR-638 gene (miRBase Accession No. MIMAT0003308) described in SEQ ID NO: 149, a homolog or an ortholog of a different organism species, and the like. The hsa-miR-638 gene can be obtained by a method described in Cummins J M et al., 2006, Proc Natl Acad Sci USA, Vol. 103, p. 3687-3692. Also, “hsa-mir-638” (miRBase Accession No. MI0003653, SEQ ID NO: 427) having a hairpin-like structure is known as a precursor of “hsa-miR-638”.

The term “hsa-miR-6741-5p gene” or “hsa-miR-6741-5p” used herein includes the hsa-miR-6741-5p gene (miRBase Accession No. MIMAT0027383) described in SEQ ID NO: 150, a homolog or an ortholog of a different organism species, and the like. The hsa-miR-6741-5p gene can be obtained by a method described in Ladewig E et al., 2012, Genome Res, Vol. 22, p. 1634-1645. Also, “hsa-mir-6741” (miRBase Accession No. MI0022586, SEQ ID NO: 428) having a hairpin-like structure is known as a precursor of “hsa-miR-6741-5p”.

The term “hsa-miR-6889-5p gene” or “hsa-miR-6889-5p” used herein includes the hsa-miR-6889-5p gene (miRBase Accession No. MIMAT0027678) described in SEQ ID NO: 151, a homolog or an ortholog of a different organism species, and the like. The hsa-miR-6889-5p gene can be obtained by a method described in Ladewig E et al., 2012, Genome Res, Vol. 22, p. 1634-1645. Also, “hsa-mir-6889” (miRBase Accession No. MI0022736, SEQ ID NO: 429) having a hairpin-like structure is known as a precursor of “hsa-miR-6889-5p”.

The term “hsa-miR-6840-3p gene” or “hsa-miR-6840-3p” used herein includes the hsa-miR-6840-3p gene (miRBase Accession No. MIMAT0027583) described in SEQ ID NO: 152, a homolog or an ortholog of a different organism species, and the like. The hsa-miR-6840-3p gene can be obtained by a method described in Ladewig E et al., 2012, Genome Res, Vol. 22, p. 1634-1645. Also, “hsa-mir-6840” (miRBase Accession No. MI0022686, SEQ ID NO: 430) having a hairpin-like structure is known as a precursor of “hsa-miR-6840-3p”.

The term “hsa-miR-6510-5p gene” or “hsa-miR-6510-5p” used herein includes the hsa-miR-6510-5p gene (miRBase Accession No. MIMAT0025476) described in SEQ ID NO: 153, a homolog or an ortholog of a different organism species, and the like. The hsa-miR-6510-5p gene can be obtained by a method described in Joyce C E et al., 2011, Hum Mol Genet, Vol. 20, p. 4025-4040. Also, “hsa-mir-6510” (miRBase Accession No. MI0022222, SEQ ID NO: 431) having a hairpin-like structure is known as a precursor of “hsa-miR-6510-5p”.

The term “hsa-miR-3188 gene” or “hsa-miR-3188” used herein includes the hsa-miR-3188 gene (miRBase Accession No. MIMAT0015070) described in SEQ ID NO: 154, a homolog or an ortholog of a different organism species, and the like. The hsa-miR-3188 gene can be obtained by a method described in Stark M S et al., 2010, PLoS One, Vol. 5, e9685. Also, “hsa-mir-3188” (miRBase Accession No. MI0014232, SEQ ID NO: 432) having a hairpin-like structure is known as a precursor of “hsa-miR-3188”.

The term “hsa-miR-551b-5p gene” or “hsa-miR-551b-5p” used herein includes the hsa-miR-551b-5p gene (miRBase Accession No. MIMAT0004794) described in SEQ ID NO: 155, a homolog or an ortholog of a different organism species, and the like. The hsa-miR-551b-5p gene can be obtained by a method described in Cummins J M et al., 2006, Proc Natl Acad Sci USA, Vol. 103, p. 3687-3692. Also, “hsa-mir-551b” (miRBase Accession No. MI0003575, SEQ ID NO: 433) having a hairpin-like structure is known as a precursor of “hsa-miR-551b-5p”.

The term “hsa-miR-5001-5p gene” or “hsa-miR-5001-5p” used herein includes the hsa-miR-5001-5p gene (miRBase Accession No. MIMAT0021021) described in SEQ ID NO: 156, a homolog or an ortholog of a different organism species, and the like. The hsa-miR-5001-5p gene can be obtained by a method described in Hansen T B et al., 2011, RNA Biol, Vol. 8, p. 378-383. Also, “hsa-mir-5001” (miRBase Accession No. MI0017867, SEQ ID NO: 434) having a hairpin-like structure is known as a precursor of “hsa-miR-5001-5p”.

The term “hsa-miR-1268b gene” or “hsa-miR-1268b” used herein includes the hsa-miR-1268b gene (miRBase Accession No. MIMAT0018925) described in SEQ ID NO: 157, a homolog or an ortholog of a different organism species, and the like. The hsa-miR-1268b gene can be obtained by a method described in Jima D D et al., 2010, Blood, Vol. 116, e118-e127. Also, “hsa-mir-1268b” (miRBase Accession No. MI0016748, SEQ ID NO: 435) having a hairpin-like structure is known as a precursor of “hsa-miR-1268b”.

The term “hsa-miR-7107-5p gene” or “hsa-miR-7107-5p” used herein includes the hsa-miR-7107-5p gene (miRBase Accession No. MIMAT0028111) described in SEQ ID NO: 158, a homolog or an ortholog of a different organism species, and the like. The hsa-miR-7107-5p gene can be obtained by a method described in Ladewig E et al., 2012, Genome Res, Vol. 22, p. 1634-1645. Also, “hsa-mir-7107” (miRBase Accession No. MI0022958, SEQ ID NO: 436) having a hairpin-like structure is known as a precursor of “hsa-miR-7107-5p”.

The term “hsa-miR-6824-5p gene” or “hsa-miR-6824-5p” used herein includes the hsa-miR-6824-5p gene (miRBase Accession No. MIMAT0027548) described in SEQ ID NO: 159, a homolog or an ortholog of a different organism species, and the like. The hsa-miR-6824-5p gene can be obtained by a method described in Ladewig E et al., 2012, Genome Res, Vol. 22, p. 1634-1645. Also, “hsa-mir-6824” (miRBase Accession No. MI0022669, SEQ ID NO: 437) having a hairpin-like structure is known as a precursor of “hsa-miR-6824-5p”.

The term “hsa-miR-6732-5p gene” or “hsa-miR-6732-5p” used herein includes the hsa-miR-6732-5p gene (miRBase Accession No. MIMAT0027365) described in SEQ ID NO: 160, a homolog or an ortholog of a different organism species, and the like. The hsa-miR-6732-5p gene can be obtained by a method described in Ladewig E et al., 2012, Genome Res, Vol. 22, p. 1634-1645. Also, “hsa-mir-6732” (miRBase Accession No. MI0022577, SEQ ID NO: 438) having a hairpin-like structure is known as a precursor of “hsa-miR-6732-5p”.

The term “hsa-miR-371a-5p gene” or “hsa-miR-371a-5p” used herein includes the hsa-miR-371a-5p gene (miRBase Accession No. MIMAT0004687) described in SEQ ID NO: 161, a homolog or an ortholog of a different organism species, and the like. The hsa-miR-371a-5p gene can be obtained by a method described in Suh M R et al., 2004, Dev Biol, Vol. 270, p. 488-498. Also, “hsa-mir-371a” (miRBase Accession No. MI0000779, SEQ ID NO: 439) having a hairpin-like structure is known as a precursor of “hsa-miR-371a-5p”.

The term “hsa-miR-6794-5p gene” or “hsa-miR-6794-5p” used herein includes the hsa-miR-6794-5p gene (miRBase Accession No. MIMAT0027488) described in SEQ ID NO: 162, a homolog or an ortholog of a different organism species, and the like. The hsa-miR-6794-5p gene can be obtained by a method described in Ladewig E et al., 2012, Genome Res, Vol. 22, p. 1634-1645. Also, “hsa-mir-6794” (miRBase Accession No. MI0022639, SEQ ID NO: 440) having a hairpin-like structure is known as a precursor of “hsa-miR-6794-5p”.

The term “hsa-miR-6779-5p gene” or “hsa-miR-6779-5p” used herein includes the hsa-miR-6779-5p gene (miRBase Accession No. MIMAT0027458) described in SEQ ID NO: 163, a homolog or an ortholog of a different organism species, and the like. The hsa-miR-6779-5p gene can be obtained by a method described in Ladewig E et al., 2012, Genome Res, Vol. 22, p. 1634-1645. Also, “hsa-mir-6779” (miRBase Accession No. MI0022624, SEQ ID NO: 441) having a hairpin-like structure is known as a precursor of “hsa-miR-6779-5p”.

The term “hsa-miR-4271 gene” or “hsa-miR-4271” used herein includes the hsa-miR-4271 gene (miRBase Accession No. MIMAT0016901) described in SEQ ID NO: 164, a homolog or an ortholog of a different organism species, and the like. The hsa-miR-4271 gene can be obtained by a method described in Goff L A et al., 2009, PLoS One, Vol. 4, e7192. Also, “hsa-mir-4271” (miRBase Accession No. MI0015879, SEQ ID NO: 442) having a hairpin-like structure is known as a precursor of “hsa-miR-4271”.

The term “hsa-miR-5195-3p gene” or “hsa-miR-5195-3p” used herein includes the hsa-miR-5195-3p gene (miRBase Accession No. MIMAT0021127) described in SEQ ID NO: 165, a homolog or an ortholog of a different organism species, and the like. The hsa-miR-5195-3p gene can be obtained by a method described in Schotte D et al., 2011, Leukemia, Vol. 25, p. 1389-1399. Also, “hsa-mir-5195” (miRBase Accession No. MI0018174, SEQ ID NO: 443) having a hairpin-like structure is known as a precursor of “hsa-miR-5195-3p”.

The term “hsa-miR-6762-5p gene” or “hsa-miR-6762-5p” used herein includes the hsa-miR-6762-5p gene (miRBase Accession No. MIMAT0027424) described in SEQ ID NO: 166, a homolog or an ortholog of a different organism species, and the like. The hsa-miR-6762-5p gene can be obtained by a method described in Ladewig E et al., 2012, Genome Res, Vol. 22, p. 1634-1645. Also, “hsa-mir-6762” (miRBase Accession No. MI0022607, SEQ ID NO: 444) having a hairpin-like structure is known as a precursor of “hsa-miR-6762-5p”.

The term “hsa-miR-939-5p gene” or “hsa-miR-939-5p” used herein includes the hsa-miR-939-5p gene (miRBase Accession No. MIMAT0004982) described in SEQ ID NO: 167, a homolog or an ortholog of a different organism species, and the like. The hsa-miR-939-5p gene can be obtained by a method described in Lui W O et al., 2007, Cancer Res, Vol. 67, p. 6031-6043. Also, “hsa-mir-939” (miRBase Accession No. MI0005761, SEQ ID NO: 445) having a hairpin-like structure is known as a precursor of “hsa-miR-939-5p”.

The term “hsa-miR-1247-3p gene” or “hsa-miR-1247-3p” used herein includes the hsa-miR-1247-3p gene (miRBase Accession No. MIMAT0022721) described in SEQ ID NO: 168, a homolog or an ortholog of a different organism species, and the like. The hsa-miR-1247-3p gene can be obtained by a method described in Morin R D et al., 2008, Genome Res, Vol. 18, p. 610-621. Also, “hsa-mir-1247” (miRBase Accession No. MI0006382, SEQ ID NO: 446) having a hairpin-like structure is known as a precursor of “hsa-miR-1247-3p”.

The term “hsa-miR-6777-5p gene” or “hsa-miR-6777-5p” used herein includes the hsa-miR-6777-5p gene (miRBase Accession No. MIMAT0027454) described in SEQ ID NO: 169, a homolog or an ortholog of a different organism species, and the like. The hsa-miR-6777-5p gene can be obtained by a method described in Ladewig E et al., 2012, Genome Res, Vol. 22, p. 1634-1645. Also, “hsa-mir-6777” (miRBase Accession No. MI0022622, SEQ ID NO: 447) having a hairpin-like structure is known as a precursor of “hsa-miR-6777-5p”.

The term “hsa-miR-6722-3p gene” or “hsa-miR-6722-3p” used herein includes the hsa-miR-6722-3p gene (miRBase Accession No. MIMAT0025854) described in SEQ ID NO: 170, a homolog or an ortholog of a different organism species, and the like. The hsa-miR-6722-3p gene can be obtained by a method described in Li Y et al., 2012, Gene, Vol. 497, p. 330-335. Also, “hsa-mir-6722” (miRBase Accession No. MI0022557, SEQ ID NO: 448) having a hairpin-like structure is known as a precursor of “hsa-miR-6722-3p”.

The term “hsa-miR-3656 gene” or “hsa-miR-3656” used herein includes the hsa-miR-3656 gene (miRBase Accession No. MIMAT0018076) described in SEQ ID NO: 171, a homolog or an ortholog of a different organism species, and the like. The hsa-miR-3656 gene can be obtained by a method described in Meiri E et al., 2010, Nucleic Acids Res, Vol. 38, p. 6234-6246. Also, “hsa-mir-3656” (miRBase Accession No. MI0016056, SEQ ID NO: 449) having a hairpin-like structure is known as a precursor of “hsa-miR-3656”.

The term “hsa-miR-4688 gene” or “hsa-miR-4688” used herein includes the hsa-miR-4688 gene (miRBase Accession No. MIMAT0019777) described in SEQ ID NO: 172, a homolog or an ortholog of a different organism species, and the like. The hsa-miR-4688 gene can be obtained by a method described in Persson H et al., 2011, Cancer Res, Vol. 71, p. 78-86. Also, “hsa-mir-4688” (miRBase Accession No. MI0017321, SEQ ID NO: 450) having a hairpin-like structure is known as a precursor of “hsa-miR-4688”.

The term “hsa-miR-3195 gene” or “hsa-miR-3195” used herein includes the hsa-miR-3195 gene (miRBase Accession No. MIMAT0015079) described in SEQ ID NO: 173, a homolog or an ortholog of a different organism species, and the like. The hsa-miR-3195 gene can be obtained by a method described in Stark M S et al., 2010, PLoS One, Vol. 5, e9685. Also, “hsa-mir-3195” (miRBase Accession No. MI0014240, SEQ ID NO: 451) having a hairpin-like structure is known as a precursor of “hsa-miR-3195”.

The term “hsa-miR-6766-5p gene” or “hsa-miR-6766-5p” used herein includes the hsa-miR-6766-5p gene (miRBase Accession No. MIMAT0027432) described in SEQ ID NO: 174, a homolog or an ortholog of a different organism species, and the like. The hsa-miR-6766-5p gene can be obtained by a method described in Ladewig E et al., 2012, Genome Res, Vol. 22, p. 1634-1645. Also, “hsa-mir-6766” (miRBase Accession No. MI0022611, SEQ ID NO: 452) having a hairpin-like structure is known as a precursor of “hsa-miR-6766-5p”.

The term “hsa-miR-4447 gene” or “hsa-miR-4447” used herein includes the hsa-miR-4447 gene (miRBase Accession No. MIMAT0018966) described in SEQ ID NO: 175, a homolog or an ortholog of a different organism species, and the like. The hsa-miR-4447 gene can be obtained by a method described in Jima D D et al., 2010, Blood, Vol. 116, e118-e127. Also, “hsa-mir-4447” (miRBase Accession No. MI0016790, SEQ ID NO: 453) having a hairpin-like structure is known as a precursor of “hsa-miR-4447”.

The term “hsa-miR-4656 gene” or “hsa-miR-4656” used herein includes the hsa-miR-4656 gene (miRBase Accession No. MIMAT0019723) described in SEQ ID NO: 176, a homolog or an ortholog of a different organism species, and the like. The hsa-miR-4656 gene can be obtained by a method described in Persson H et al., 2011, Cancer Res, Vol. 71, p. 78-86. Also, “hsa-mir-4656” (miRBase Accession No. MI0017284, SEQ ID NO: 454) having a hairpin-like structure is known as a precursor of “hsa-miR-4656”.

The term “hsa-miR-7108-5p gene” or “hsa-miR-7108-5p” used herein includes the hsa-miR-7108-5p gene (miRBase Accession No. MIMAT0028113) described in SEQ ID NO: 177, a homolog or an ortholog of a different organism species, and the like. The hsa-miR-7108-5p gene can be obtained by a method described in Ladewig E et al., 2012, Genome Res, Vol. 22, p. 1634-1645. Also, “hsa-mir-7108” (miRBase Accession No. MI0022959, SEQ ID NO: 455) having a hairpin-like structure is known as a precursor of “hsa-miR-7108-5p”.

The term “hsa-miR-3191-3p gene” or “hsa-miR-3191-3p” used herein includes the hsa-miR-3191-3p gene (miRBase Accession No. MIMAT0015075) described in SEQ ID NO: 178, a homolog or an ortholog of a different organism species, and the like. The hsa-miR-3191-3p gene can be obtained by a method described in Stark M S et al., 2010, PLoS One, Vol. 5, e9685. Also, “hsa-mir-3191” (miRBase Accession No. MI0014236, SEQ ID NO: 456) having a hairpin-like structure is known as a precursor of “hsa-miR-3191-3p”.

The term “hsa-miR-1273g-3p gene” or “hsa-miR-1273g-3p” used herein includes the hsa-miR-1273g-3p gene (miRBase Accession No. MIMAT0022742) described in SEQ ID NO: 179, a homolog or an ortholog of a different organism species, and the like. The hsa-miR-1273g-3p gene can be obtained by a method described in Reshmi G et al., 2011, Genomics, Vol. 97, p. 333-340. Also, “hsa-mir-1273g” (miRBase Accession No. MI0018003, SEQ ID NO: 457) having a hairpin-like structure is known as a precursor of “hsa-miR-1273g-3p”.

The term “hsa-miR-4463 gene” or “hsa-miR-4463” used herein includes the hsa-miR-4463 gene (miRBase Accession No. MIMAT0018987) described in SEQ ID NO: 180, a homolog or an ortholog of a different organism species, and the like. The hsa-miR-4463 gene can be obtained by a method described in lima D D et al., 2010, Blood, Vol. 116, e118-e127. Also, “hsa-mir-4463” (miRBase Accession No. MI0016811, SEQ ID NO: 458) having a hairpin-like structure is known as a precursor of “hsa-miR-4463”.

The term “hsa-miR-2861 gene” or “hsa-miR-2861” used herein includes the hsa-miR-2861 gene (miRBase Accession No. MIMAT0013802) described in SEQ ID NO: 181, a homolog or an ortholog of a different organism species, and the like. The hsa-miR-2861 gene can be obtained by a method described in Li H et al., 2009, 1 Clin Invest, Vol. 119, p. 3666-3677. Also, “hsa-mir-2861” (miRBase Accession No. MI0013006, SEQ ID NO: 459) having a hairpin-like structure is known as a precursor of “hsa-miR-2861”.

The term “hsa-miR-3196 gene” or “hsa-miR-3196” used herein includes the hsa-miR-3196 gene (miRBase Accession No. MIMAT0015080) described in SEQ ID NO: 182, a homolog or an ortholog of a different organism species, and the like. The hsa-miR-3196 gene can be obtained by a method described in Stark M S et al., 2010, PLoS One, Vol. 5, e9685. Also, “hsa-mir-3196” (miRBase Accession No. MI0014241, SEQ ID NO: 460) having a hairpin-like structure is known as a precursor of “hsa-miR-3196”.

The term “hsa-miR-6877-5p gene” or “hsa-miR-6877-5p” used herein includes the hsa-miR-6877-5p gene (miRBase Accession No. MIMAT0027654) described in SEQ ID NO: 183, a homolog or an ortholog of a different organism species, and the like. The hsa-miR-6877-5p gene can be obtained by a method described in Ladewig E et al., 2012, Genome Res, Vol. 22, p. 1634-1645. Also, “hsa-mir-6877” (miRBase Accession No. MI0022724, SEQ ID NO: 461) having a hairpin-like structure is known as a precursor of “hsa-miR-6877-5p”.

The term “hsa-miR-3679-5p gene” or “hsa-miR-3679-5p” used herein includes the hsa-miR-3679-5p gene (miRBase Accession No. MIMAT0018104) described in SEQ ID NO: 184, a homolog or an ortholog of a different organism species, and the like. The hsa-miR-3679-5p gene can be obtained by a method described in Creighton C J et al., 2010, PLoS One, Vol. 5, e9637. Also, “hsa-mir-3679” (miRBase Accession No. MI0016080, SEQ ID NO: 462) having a hairpin-like structure is known as a precursor of “hsa-miR-3679-5p”.

The term “hsa-miR-4442 gene” or “hsa-miR-4442” used herein includes the hsa-miR-4442 gene (miRBase Accession No. MIMAT0018960) described in SEQ ID NO: 185, a homolog or an ortholog of a different organism species, and the like. The hsa-miR-4442 gene can be obtained by a method described in Jima D D et al., 2010, Blood, Vol. 116, e118-e127. Also, “hsa-mir-4442” (miRBase Accession No. MI0016785, SEQ ID NO: 463) having a hairpin-like structure is known as a precursor of “hsa-miR-4442”.

The term “hsa-miR-6789-5p gene” or “hsa-miR-6789-5p” used herein includes the hsa-miR-6789-5p gene (miRBase Accession No. MIMAT0027478) described in SEQ ID NO: 186, a homolog or an ortholog of a different organism species, and the like. The hsa-miR-6789-5p gene can be obtained by a method described in Ladewig E et al., 2012, Genome Res, Vol. 22, p. 1634-1645. Also, “hsa-mir-6789” (miRBase Accession No. MI0022634, SEQ ID NO: 464) having a hairpin-like structure is known as a precursor of “hsa-miR-6789-5p”.

The term “hsa-miR-6782-5p gene” or “hsa-miR-6782-5p” used herein includes the hsa-miR-6782-5p gene (miRBase Accession No. MIMAT0027464) described in SEQ ID NO: 187, a homolog or an ortholog of a different organism species, and the like. The hsa-miR-6782-5p gene can be obtained by a method described in Ladewig E et al., 2012, Genome Res, Vol. 22, p. 1634-1645. Also, “hsa-mir-6782” (miRBase Accession No. MI0022627, SEQ ID NO: 465) having a hairpin-like structure is known as a precursor of “hsa-miR-6782-5p”.

The term “hsa-miR-486-3p gene” or “hsa-miR-486-3p” used herein includes the hsa-miR-486-3p gene (miRBase Accession No. MIMAT0004762) described in SEQ ID NO: 188, a homolog or an ortholog of a different organism species, and the like. The hsa-miR-486-3p gene can be obtained by a method described in Fu H et al., 2005, FEBS Lett, Vol. 579, p. 3849-3854. Also, “hsa-mir-486 and hsa-mir-486-2” (miRBase Accession Nos. MI0002470 and MI0023622, SEQ ID NOs: 466 and 467) having a hairpin-like structure are known as precursors of “hsa-miR-486-3p”.

The term “hsa-miR-6085 gene” or “hsa-miR-6085” used herein includes the hsa-miR-6085 gene (miRBase Accession No. MIMAT0023710) described in SEQ ID NO: 189, a homolog or an ortholog of a different organism species, and the like. The hsa-miR-6085 gene can be obtained by a method described in Voellenkle C et al., 2012, RNA, Vol. 18, p. 472-484. Also, “hsa-mir-6085” (miRBase Accession No. MI0020362, SEQ ID NO: 468) having a hairpin-like structure is known as a precursor of “hsa-miR-6085”.

The term “hsa-miR-4746-3p gene” or “hsa-miR-4746-3p” used herein includes the hsa-miR-4746-3p gene (miRBase Accession No. MIMAT0019881) described in SEQ ID NO: 190, a homolog or an ortholog of a different organism species, and the like. The hsa-miR-4746-3p gene can be obtained by a method described in Persson H et al., 2011, Cancer Res, Vol. 71, p. 78-86. Also, “hsa-mir-4746” (miRBase Accession No. MI0017385, SEQ ID NO: 469) having a hairpin-like structure is known as a precursor of “hsa-miR-4746-3p”.

The term “hsa-miR-619-5p gene” or “hsa-miR-619-5p” used herein includes the hsa-miR-619-5p gene (miRBase Accession No. MIMAT0026622) described in SEQ ID NO: 191, a homolog or an ortholog of a different organism species, and the like. The hsa-miR-619-5p gene can be obtained by a method described in Cummins J M et al., 2006, Proc Natl Acad Sci USA, Vol. 103, p. 3687-3692. Also, “hsa-mir-619” (miRBase Accession No. MI0003633, SEQ ID NO: 470) having a hairpin-like structure is known as a precursor of “hsa-miR-619-5p”.

The term “hsa-miR-937-5p gene” or “hsa-miR-937-5p” used herein includes the hsa-miR-937-5p gene (miRBase Accession No. MIMAT0022938) described in SEQ ID NO: 192, a homolog or an ortholog of a different organism species, and the like. The hsa-miR-937-5p gene can be obtained by a method described in Lui W O et al., 2007, Cancer Res, Vol. 67, p. 6031-6043. Also, “hsa-mir-937” (miRBase Accession No. MI0005759, SEQ ID NO: 471) having a hairpin-like structure is known as a precursor of “hsa-miR-937-5p”.

The term “hsa-miR-6803-5p gene” or “hsa-miR-6803-5p” used herein includes the hsa-miR-6803-5p gene (miRBase Accession No. MIMAT0027506) described in SEQ ID NO: 193, a homolog or an ortholog of a different organism species, and the like. The hsa-miR-6803-5p gene can be obtained by a method described in Ladewig E et al., 2012, Genome Res, Vol. 22, p. 1634-1645. Also, “hsa-mir-6803” (miRBase Accession No. MI0022648, SEQ ID NO: 472) having a hairpin-like structure is known as a precursor of “hsa-miR-6803-5p”.

The term “hsa-miR-4298 gene” or “hsa-miR-4298” used herein includes the hsa-miR-4298 gene (miRBase Accession No. MIMAT0016852) described in SEQ ID NO: 194, a homolog or an ortholog of a different organism species, and the like. The hsa-miR-4298 gene can be obtained by a method described in Goff L A et al., 2009, PLoS One, Vol. 4, e7192. Also, “hsa-mir-4298” (miRBase Accession No. MI0015830, SEQ ID NO: 473) having a hairpin-like structure is known as a precursor of “hsa-miR-4298”.

The term “hsa-miR-4454 gene” or “hsa-miR-4454” used herein includes the hsa-miR-4454 gene (miRBase Accession No. MIMAT0018976) described in SEQ ID NO: 195, a homolog or an ortholog of a different organism species, and the like. The hsa-miR-4454 gene can be obtained by a method described in Jima D D et al., 2010, Blood, Vol. 116, e118-e127. Also, “hsa-mir-4454” (miRBase Accession No. MI0016800, SEQ ID NO: 474) having a hairpin-like structure is known as a precursor of “hsa-miR-4454”.

The term “hsa-miR-4459 gene” or “hsa-miR-4459” used herein includes the hsa-miR-4459 gene (miRBase Accession No. MIMAT0018981) described in SEQ ID NO: 196, a homolog or an ortholog of a different organism species, and the like. The hsa-miR-4459 gene can be obtained by a method described in lima D D et al., 2010, Blood, Vol. 116, e118-e127. Also, “hsa-mir-4459” (miRBase Accession No. MI0016805, SEQ ID NO: 475) having a hairpin-like structure is known as a precursor of “hsa-miR-4459”.

The term “hsa-miR-7150 gene” or “hsa-miR-7150” used herein includes the hsa-miR-7150 gene (miRBase Accession No. MIMAT0028211) described in SEQ ID NO: 197, a homolog or an ortholog of a different organism species, and the like. The hsa-miR-7150 gene can be obtained by a method described in Oulas A et al., 2009, Nucleic Acids Res, Vol. 37, p. 3276-3287. Also, “hsa-mir-7150” (miRBase Accession No. MI0023610, SEQ ID NO: 476) having a hairpin-like structure is known as a precursor of “hsa-miR-7150”.

The term “hsa-miR-6880-5p gene” or “hsa-miR-6880-5p” used herein includes the hsa-miR-6880-5p gene (miRBase Accession No. MIMAT0027660) described in SEQ ID NO: 198, a homolog or an ortholog of a different organism species, and the like. The hsa-miR-6880-5p gene can be obtained by a method described in Ladewig E et al., 2012, Genome Res, Vol. 22, p. 1634-1645. Also, “hsa-mir-6880” (miRBase Accession No. MI0022727, SEQ ID NO: 477) having a hairpin-like structure is known as a precursor of “hsa-miR-6880-5p”.

The term “hsa-miR-4449 gene” or “hsa-miR-4449” used herein includes the hsa-miR-4449 gene (miRBase Accession No. MIMAT0018968) described in SEQ ID NO: 199, a homolog or an ortholog of a different organism species, and the like. The hsa-miR-4449 gene can be obtained by a method described in Jima D D et al., 2010, Blood, Vol. 116, e118-e127. Also, “hsa-mir-4449” (miRBase Accession No. MI0016792, SEQ ID NO: 478) having a hairpin-like structure is known as a precursor of “hsa-miR-4449”.

The term “hsa-miR-8063 gene” or “hsa-miR-8063” used herein includes the hsa-miR-8063 gene (miRBase Accession No. MIMAT0030990) described in SEQ ID NO: 200, a homolog or an ortholog of a different organism species, and the like. The hsa-miR-8063 gene can be obtained by a method described in Wang H J et al., 2013, Shock, Vol. 39, p. 480-487. Also, “hsa-mir-8063” (miRBase Accession No. MI0025899, SEQ ID NO: 479) having a hairpin-like structure is known as a precursor of “hsa-miR-8063”.

The term “hsa-miR-4695-5p gene” or “hsa-miR-4695-5p” used herein includes the hsa-miR-4695-5p gene (miRBase Accession No. MIMAT0019788) described in SEQ ID NO: 201, a homolog or an ortholog of a different organism species, and the like. The hsa-miR-4695-5p gene can be obtained by a method described in Persson H et al., 2011, Cancer Res, Vol. 71, p. 78-86. Also, “hsa-mir-4695” (miRBase Accession No. MI0017328, SEQ ID NO: 480) having a hairpin-like structure is known as a precursor of “hsa-miR-4695-5p”.

The term “hsa-miR-6132 gene” or “hsa-miR-6132” used herein includes the hsa-miR-6132 gene (miRBase Accession No. MIMAT0024616) described in SEQ ID NO: 202, a homolog or an ortholog of a different organism species, and the like. The hsa-miR-6132 gene can be obtained by a method described in Dannemann M et al., 2012, Genome Biol Evol, Vol. 4, p. 552-564. Also, “hsa-mir-6132” (miRBase Accession No. MI0021277, SEQ ID NO: 481) having a hairpin-like structure is known as a precursor of “hsa-miR-6132”.

The term “hsa-miR-6829-5p gene” or “hsa-miR-6829-5p” used herein includes the hsa-miR-6829-5p gene (miRBase Accession No. MIMAT0027558) described in SEQ ID NO: 203, a homolog or an ortholog of a different organism species, and the like. The hsa-miR-6829-5p gene can be obtained by a method described in Ladewig E et al., 2012, Genome Res, Vol. 22, p. 1634-1645. Also, “hsa-mir-6829” (miRBase Accession No. MI0022674, SEQ ID NO: 482) having a hairpin-like structure is known as a precursor of “hsa-miR-6829-5p”.

The term “hsa-miR-4486 gene” or “hsa-miR-4486” used herein includes the hsa-miR-4486 gene (miRBase Accession No. MIMAT0019020) described in SEQ ID NO: 204, a homolog or an ortholog of a different organism species, and the like. The hsa-miR-4486 gene can be obtained by a method described in lima D D et al., 2010, Blood, Vol. 116, e118-e127. Also, “hsa-mir-4486” (miRBase Accession No. MI0016847, SEQ ID NO: 483) having a hairpin-like structure is known as a precursor of “hsa-miR-4486”.

The term “hsa-miR-6805-3p gene” or “hsa-miR-6805-3p” used herein includes the hsa-miR-6805-3p gene (miRBase Accession No. MIMAT0027511) described in SEQ ID NO: 205, a homolog or an ortholog of a different organism species, and the like. The hsa-miR-6805-3p gene can be obtained by a method described in Ladewig E et al., 2012, Genome Res, Vol. 22, p. 1634-1645. Also, “hsa-mir-6805” (miRBase Accession No. MI0022650, SEQ ID NO: 304) having a hairpin-like structure is known as a precursor of “hsa-miR-6805-3p”.

The term “hsa-miR-6826-5p gene” or “hsa-miR-6826-5p” used herein includes the hsa-miR-6826-5p gene (miRBase Accession No. MIMAT0027552) described in SEQ ID NO: 206, a homolog or an ortholog of a different organism species, and the like. The hsa-miR-6826-5p gene can be obtained by a method described in Ladewig E et al., 2012, Genome Res, Vol. 22, p. 1634-1645. Also, “hsa-mir-6826” (miRBase Accession No. MI0022671, SEQ ID NO: 484) having a hairpin-like structure is known as a precursor of “hsa-miR-6826-5p”.

The term “hsa-miR-4508 gene” or “hsa-miR-4508” used herein includes the hsa-miR-4508 gene (miRBase Accession No. MIMAT0019045) described in SEQ ID NO: 207, a homolog or an ortholog of a different organism species, and the like. The hsa-miR-4508 gene can be obtained by a method described in Jima D D et al., 2010, Blood, Vol. 116, e118-e127. Also, “hsa-mir-4508” (miRBase Accession No. MI0016872, SEQ ID NO: 485) having a hairpin-like structure is known as a precursor of “hsa-miR-4508”.

The term “hsa-miR-1343-5p gene” or “hsa-miR-1343-5p” used herein includes the hsa-miR-1343-5p gene (miRBase Accession No. MIMAT0027038) described in SEQ ID NO: 208, a homolog or an ortholog of a different organism species, and the like. The hsa-miR-1343-5p gene can be obtained by a method described in Persson H et al., 2011, Cancer Res, Vol. 71, p. 78-86. Also, “hsa-mir-1343” (miRBase Accession No. MI0017320, SEQ ID NO: 361) having a hairpin-like structure is known as a precursor of “hsa-miR-1343-5p”.

The term “hsa-miR-7114-5p gene” or “hsa-miR-7114-5p” used herein includes the hsa-miR-7114-5p gene (miRBase Accession No. MIMAT0028125) described in SEQ ID NO: 209, a homolog or an ortholog of a different organism species, and the like. The hsa-miR-7114-5p gene can be obtained by a method described in Ladewig E et al., 2012, Genome Res, Vol. 22, p. 1634-1645. Also, “hsa-mir-7114” (miRBase Accession No. MI0022965, SEQ ID NO: 486) having a hairpin-like structure is known as a precursor of “hsa-miR-7114-5p”.

The term “hsa-miR-3622a-5p gene” or “hsa-miR-3622a-5p” used herein includes the hsa-miR-3622a-5p gene (miRBase Accession No. MIMAT0018003) described in SEQ ID NO: 210, a homolog or an ortholog of a different organism species, and the like. The hsa-miR-3622a-5p gene can be obtained by a method described in Witten D et al., 2010, BMC Biol, Vol. 8, p. 58. Also, “hsa-mir-3622a” (miRBase Accession No. MI0016013, SEQ ID NO: 487) having a hairpin-like structure is known as a precursor of “hsa-miR-3622a-5p”.

The term “hsa-miR-6765-5p gene” or “hsa-miR-6765-5p” used herein includes the hsa-miR-6765-5p gene (miRBase Accession No. MIMAT0027430) described in SEQ ID NO: 211, a homolog or an ortholog of a different organism species, and the like. The hsa-miR-6765-5p gene can be obtained by a method described in Ladewig E et al., 2012, Genome Res, Vol. 22, p. 1634-1645. Also, “hsa-mir-6765” (miRBase Accession No. MI0022610, SEQ ID NO: 488) having a hairpin-like structure is known as a precursor of “hsa-miR-6765-5p”.

The term “hsa-miR-7845-5p gene” or “hsa-miR-7845-5p” used herein includes the hsa-miR-7845-5p gene (miRBase Accession No. MIMAT0030420) described in SEQ ID NO: 212, a homolog or an ortholog of a different organism species, and the like. The hsa-miR-7845-5p gene can be obtained by a method described in Pie H et al., 2012, PLoS One, Vol. 7, e50746. Also, “hsa-mir-7845” (miRBase Accession No. MI0025515, SEQ ID NO: 489) having a hairpin-like structure is known as a precursor of “hsa-miR-7845-5p”.

The term “hsa-miR-3960 gene” or “hsa-miR-3960” used herein includes the hsa-miR-3960 gene (miRBase Accession No. MIMAT0019337) described in SEQ ID NO: 213, a homolog or an ortholog of a different organism species, and the like. The hsa-miR-3960 gene can be obtained by a method described in Hu R et al., 2011, J Biol Chem, Vol. 286, p. 12328-12339. Also, “hsa-mir-3960” (miRBase Accession No. MI0016964, SEQ ID NO: 490) having a hairpin-like structure is known as a precursor of “hsa-miR-3960”.

The term “hsa-miR-6749-5p gene” or “hsa-miR-6749-5p” used herein includes the hsa-miR-6749-5p gene (miRBase Accession No. MIMAT0027398) described in SEQ ID NO: 214, a homolog or an ortholog of a different organism species, and the like. The hsa-miR-6749-5p gene can be obtained by a method described in Ladewig E et al., 2012, Genome Res, Vol. 22, p. 1634-1645. Also, “hsa-mir-6749” (miRBase Accession No. MI0022594, SEQ ID NO: 491) having a hairpin-like structure is known as a precursor of “hsa-miR-6749-5p”.

The term “hsa-miR-1260b gene” or “hsa-miR-1260b” used herein includes the hsa-miR-1260b gene (miRBase Accession No. MIMAT0015041) described in SEQ ID NO: 215, a homolog or an ortholog of a different organism species, and the like. The hsa-miR-1260b gene can be obtained by a method described in Stark M S et al., 2010, PLoS One, Vol. 5, e9685. Also, “hsa-mir-1260b” (miRBase Accession No. MI0014197, SEQ ID NO: 492) having a hairpin-like structure is known as a precursor of “hsa-miR-1260b”.

The term “hsa-miR-6799-5p gene” or “hsa-miR-6799-5p” used herein includes the hsa-miR-6799-5p gene (miRBase Accession No. MIMAT0027498) described in SEQ ID NO: 216, a homolog or an ortholog of a different organism species, and the like. The hsa-miR-6799-5p gene can be obtained by a method described in Ladewig E et al., 2012, Genome Res, Vol. 22, p. 1634-1645. Also, “hsa-mir-6799” (miRBase Accession No. MI0022644, SEQ ID NO: 493) having a hairpin-like structure is known as a precursor of “hsa-miR-6799-5p”.

The term “hsa-miR-4723-5p gene” or “hsa-miR-4723-5p” used herein includes the hsa-miR-4723-5p gene (miRBase Accession No. MIMAT0019838) described in SEQ ID NO: 217, a homolog or an ortholog of a different organism species, and the like. The hsa-miR-4723-5p gene can be obtained by a method described in Persson H et al., 2011, Cancer Res, Vol. 71, p. 78-86. Also, “hsa-mir-4723” (miRBase Accession No. MI0017359, SEQ ID NO: 494) having a hairpin-like structure is known as a precursor of “hsa-miR-4723-5p”.

The term “hsa-miR-6784-5p gene” or “hsa-miR-6784-5p” used herein includes the hsa-miR-6784-5p gene (miRBase Accession No. MIMAT0027468) described in SEQ ID NO: 218, a homolog or an ortholog of a different organism species, and the like. The hsa-miR-6784-5p gene can be obtained by a method described in Ladewig E et al., 2012, Genome Res, Vol. 22, p. 1634-1645. Also, “hsa-mir-6784” (miRBase Accession No. MI0022629, SEQ ID NO: 495) having a hairpin-like structure is known as a precursor of “hsa-miR-6784-5p”.

The term “hsa-miR-5100 gene” or “hsa-miR-5100” used herein includes the hsa-miR-5100 gene (miRBase Accession No. MIMAT0022259) described in SEQ ID NO: 219, a homolog or an ortholog of a different organism species, and the like. The hsa-miR-5100 gene can be obtained by a method described in Tandon M et al., 2012, Oral Dis, Vol. 18, p. 127-131. Also, “hsa-mir-5100” (miRBase Accession No. MI0019116, SEQ ID NO: 496) having a hairpin-like structure is known as a precursor of “hsa-miR-5100”.

The term “hsa-miR-6769b-5p gene” or “hsa-miR-6769b-5p” used herein includes the hsa-miR-6769b-5p gene (miRBase Accession No. MIMAT0027620) described in SEQ ID NO: 220, a homolog or an ortholog of a different organism species, and the like. The hsa-miR-6769b-5p gene can be obtained by a method described in Ladewig E et al., 2012, Genome Res, Vol. 22, p. 1634-1645. Also, “hsa-mir-6769b” (miRBase Accession No. MI0022706, SEQ ID NO: 497) having a hairpin-like structure is known as a precursor of “hsa-miR-6769b-5p”.

The term “hsa-miR-1207-5p gene” or “hsa-miR-1207-5p” used herein includes the hsa-miR-1207-5p gene (miRBase Accession No. MIMAT0005871) described in SEQ ID NO: 221, a homolog or an ortholog of a different organism species, and the like. The hsa-miR-1207-5p gene can be obtained by a method described in Huppi K et al., 2008, Mol Cancer Res, Vol. 6, p. 212-221. Also, “hsa-mir-1207” (miRBase Accession No. MI0006340, SEQ ID NO: 498) having a hairpin-like structure is known as a precursor of “hsa-miR-1207-5p”.

The term “hsa-miR-642a-3p gene” or “hsa-miR-642a-3p” used herein includes the hsa-miR-642a-3p gene (miRBase Accession No. MIMAT0020924) described in SEQ ID NO: 222, a homolog or an ortholog of a different organism species, and the like. The hsa-miR-642a-3p gene can be obtained by a method described in Cummins J M et al., 2006, Proc Natl Acad Sci USA, Vol. 103, p. 3687-3692. Also, “hsa-mir-642a” (miRBase Accession No. MI0003657, SEQ ID NO: 499) having a hairpin-like structure is known as a precursor of “hsa-miR-642a-3p”.

The term “hsa-miR-4505 gene” or “hsa-miR-4505” used herein includes the hsa-miR-4505 gene (miRBase Accession No. MIMAT0019041) described in SEQ ID NO: 223, a homolog or an ortholog of a different organism species, and the like. The hsa-miR-4505 gene can be obtained by a method described in Jima D D et al., 2010, Blood, Vol. 116, e118-e127. Also, “hsa-mir-4505” (miRBase Accession No. MI0016868, SEQ ID NO: 500) having a hairpin-like structure is known as a precursor of “hsa-miR-4505”.

The term “hsa-miR-4270 gene” or “hsa-miR-4270” used herein includes the hsa-miR-4270 gene (miRBase Accession No. MIMAT0016900) described in SEQ ID NO: 224, a homolog or an ortholog of a different organism species, and the like. The hsa-miR-4270 gene can be obtained by a method described in Goff L A et al., 2009, PLoS One, Vol. 4, e7192. Also, “hsa-mir-4270” (miRBase Accession No. MI0015878, SEQ ID NO: 501) having a hairpin-like structure is known as a precursor of “hsa-miR-4270”.

The term “hsa-miR-6721-5p gene” or “hsa-miR-6721-5p” used herein includes the hsa-miR-6721-5p gene (miRBase Accession No. MIMAT0025852) described in SEQ ID NO: 225, a homolog or an ortholog of a different organism species, and the like. The hsa-miR-6721-5p gene can be obtained by a method described in Li Y et al., 2012, Gene, Vol. 497, p. 330-335. Also, “hsa-mir-6721” (miRBase Accession No. MI0022556, SEQ ID NO: 502) having a hairpin-like structure is known as a precursor of “hsa-miR-6721-5p”.

The term “hsa-miR-7111-5p gene” or “hsa-miR-7111-5p” used herein includes the hsa-miR-7111-5p gene (miRBase Accession No. MIMAT0028119) described in SEQ ID NO: 226, a homolog or an ortholog of a different organism species, and the like. The hsa-miR-7111-5p gene can be obtained by a method described in Ladewig E et al., 2012, Genome Res, Vol. 22, p. 1634-1645. Also, “hsa-mir-7111” (miRBase Accession No. MI0022962, SEQ ID NO: 503) having a hairpin-like structure is known as a precursor of “hsa-miR-7111-5p”.

The term “hsa-miR-6791-5p gene” or “hsa-miR-6791-5p” used herein includes the hsa-miR-6791-5p gene (miRBase Accession No. MIMAT0027482) described in SEQ ID NO: 227, a homolog or an ortholog of a different organism species, and the like. The hsa-miR-6791-5p gene can be obtained by a method described in Ladewig E et al., 2012, Genome Res, Vol. 22, p. 1634-1645. Also, “hsa-mir-6791” (miRBase Accession No. MI0022636, SEQ ID NO: 504) having a hairpin-like structure is known as a precursor of “hsa-miR-6791-5p”.

The term “hsa-miR-7109-5p gene” or “hsa-miR-7109-5p” used herein includes the hsa-miR-7109-5p gene (miRBase Accession No. MIMAT0028115) described in SEQ ID NO: 228, a homolog or an ortholog of a different organism species, and the like. The hsa-miR-7109-5p gene can be obtained by a method described in Ladewig E et al., 2012, Genome Res, Vol. 22, p. 1634-1645. Also, “hsa-mir-7109” (miRBase Accession No. MI0022960, SEQ ID NO: 505) having a hairpin-like structure is known as a precursor of “hsa-miR-7109-5p”.

The term “hsa-miR-4258 gene” or “hsa-miR-4258” used herein includes the hsa-miR-4258 gene (miRBase Accession No. MIMAT0016879) described in SEQ ID NO: 229, a homolog or an ortholog of a different organism species, and the like. The hsa-miR-4258 gene can be obtained by a method described in Goff L A et al., 2009, PLoS One, Vol. 4, e7192. Also, “hsa-mir-4258” (miRBase Accession No. MI0015857, SEQ ID NO: 506) having a hairpin-like structure is known as a precursor of “hsa-miR-4258”.

The term “hsa-miR-6515-3p gene” or “hsa-miR-6515-3p” used herein includes the hsa-miR-6515-3p gene (miRBase Accession No. MIMAT0025487) described in SEQ ID NO: 230, a homolog or an ortholog of a different organism species, and the like. The hsa-miR-6515-3p gene can be obtained by a method described in Joyce C E et al., 2011, Hum Mol Genet, Vol. 20, p. 4025-4040. Also, “hsa-mir-6515” (miRBase Accession No. MI0022227, SEQ ID NO: 507) having a hairpin-like structure is known as a precursor of “hsa-miR-6515-3p”.

The term “hsa-miR-6851-5p gene” or “hsa-miR-6851-5p” used herein includes the hsa-miR-6851-5p gene (miRBase Accession No. MIMAT0027602) described in SEQ ID NO: 231, a homolog or an ortholog of a different organism species, and the like. The hsa-miR-6851-5p gene can be obtained by a method described in Ladewig E et al., 2012, Genome Res, Vol. 22, p. 1634-1645. Also, “hsa-mir-6851” (miRBase Accession No. MI0022697, SEQ ID NO: 508) having a hairpin-like structure is known as a precursor of “hsa-miR-6851-5p”.

The term “hsa-miR-6125 gene” or “hsa-miR-6125” used herein includes the hsa-miR-6125 gene (miRBase Accession No. MIMAT0024598) described in SEQ ID NO: 232, a homolog or an ortholog of a different organism species, and the like. The hsa-miR-6125 gene can be obtained by a method described in Smith J L et al., 2012, J Virol, Vol. 86, p. 5278-5287. Also, “hsa-mir-6125” (miRBase Accession No. MI0021259, SEQ ID NO: 509) having a hairpin-like structure is known as a precursor of “hsa-miR-6125”.

The term “hsa-miR-4749-5p gene” or “hsa-miR-4749-5p” used herein includes the hsa-miR-4749-5p gene (miRBase Accession No. MIMAT0019885) described in SEQ ID NO: 233, a homolog or an ortholog of a different organism species, and the like. The hsa-miR-4749-5p gene can be obtained by a method described in Persson H et al., 2011, Cancer Res, Vol. 71, p. 78-86. Also, “hsa-mir-4749” (miRBase Accession No. MI0017388, SEQ ID NO: 510) having a hairpin-like structure is known as a precursor of “hsa-miR-4749-5p”.

The term “hsa-miR-4726-5p gene” or “hsa-miR-4726-5p” used herein includes the hsa-miR-4726-5p gene (miRBase Accession No. MIMAT0019845) described in SEQ ID NO: 234, a homolog or an ortholog of a different organism species, and the like. The hsa-miR-4726-5p gene can be obtained by a method described in Persson H et al., 2011, Cancer Res, Vol. 71, p. 78-86. Also, “hsa-mir-4726” (miRBase Accession No. MI0017363, SEQ ID NO: 511) having a hairpin-like structure is known as a precursor of “hsa-miR-4726-5p”.

The term “hsa-miR-4513 gene” or “hsa-miR-4513” used herein includes the hsa-miR-4513 gene (miRBase Accession No. MIMAT0019050) described in SEQ ID NO: 235, a homolog or an ortholog of a different organism species, and the like. The hsa-miR-4513 gene can be obtained by a method described in Jima D D et al., 2010, Blood, Vol. 116, e118-e127. Also, “hsa-mir-4513” (miRBase Accession No. MI0016879, SEQ ID NO: 512) having a hairpin-like structure is known as a precursor of “hsa-miR-4513”.

The term “hsa-miR-760 gene” or “hsa-miR-760” used herein includes the hsa-miR-760 gene (miRBase Accession No. MIMAT0004957) described in SEQ ID NO: 236, a homolog or an ortholog of a different organism species, and the like. The hsa-miR-760 gene can be obtained by a method described in Berezikov E et al., 2006, Genome Res, Vol. 16, p. 1289-1298. Also, “hsa-mir-760” (miRBase Accession No. MI0005567, SEQ ID NO: 513) having a hairpin-like structure is known as a precursor of “hsa-miR-760”.

The term “hsa-miR-602 gene” or “hsa-miR-602” used herein includes the hsa-miR-602 gene (miRBase Accession No. MIMAT0003270) described in SEQ ID NO: 237, a homolog or an ortholog of a different organism species, and the like. The hsa-miR-602 gene can be obtained by a method described in Cummins J M et al., 2006, Proc Natl Acad Sci USA, Vol. 103, p. 3687-3692. Also, “hsa-mir-602” (miRBase Accession No. MI0003615, SEQ ID NO: 514) having a hairpin-like structure is known as a precursor of “hsa-miR-602”.

The term “hsa-miR-423-5p gene” or “hsa-miR-423-5p” used herein includes the hsa-miR-423-5p gene (miRBase Accession No. MIMAT0004748) described in SEQ ID NO: 238, a homolog or an ortholog of a different organism species, and the like. The hsa-miR-423-5p gene can be obtained by a method described in Kasashima K et al., 2004, Biochem Biophys Res Commun, Vol. 322, p. 403-410. Also, “hsa-mir-423” (miRBase Accession No. MI0001445, SEQ ID NO: 515) having a hairpin-like structure is known as a precursor of “hsa-miR-423-5p”.

The term “hsa-miR-92a-2-5p gene” or “hsa-miR-92a-2-5p” used herein includes the hsa-miR-92a-2-5p gene (miRBase Accession No. MIMAT0004508) described in SEQ ID NO: 239, a homolog or an ortholog of a different organism species, and the like. The hsa-miR-92a-2-5p gene can be obtained by a method described in Mourelatos Z et al., 2002, Genes Dev, Vol. 16, p. 720-728. Also, “hsa-mir-92a-2” (miRBase Accession No. MI0000094, SEQ ID NO: 516) having a hairpin-like structure is known as a precursor of “hsa-miR-92a-2-5p”.

The term “hsa-miR-16-5p gene” or “hsa-miR-16-5p” used herein includes the hsa-miR-16-5p gene (miRBase Accession No. MIMAT0000069) described in SEQ ID NO: 240, a homolog or an ortholog of a different organism species, and the like. The hsa-miR-16-5p gene can be obtained by a method described in Lagos-Quintana M et al., 2001, Science, Vol. 294, p. 853-858. Also, “hsa-mir-16-1 and hsa-mir-16-2” (miRBase Accession Nos. MI0000070 and MI0000115, SEQ ID NOs: 517 and 518) having a hairpin-like structure are known as precursors of “hsa-miR-16-5p”.

The term “hsa-miR-451a gene” or “hsa-miR-451a” used herein includes the hsa-miR-451a gene (miRBase Accession No. MIMAT0001631) described in SEQ ID NO: 241, a homolog or an ortholog of a different organism species, and the like. The hsa-miR-451a gene can be obtained by a method described in Altuvia Y et al., 2005, Nucleic Acids Res, Vol. 33, p. 2697-2706. Also, “hsa-mir-451a” (miRBase Accession No. MI0001729, SEQ ID NO: 519) having a hairpin-like structure is known as a precursor of “hsa-miR-451a”.

The term “hsa-miR-135a-3p gene” or “hsa-miR-135a-3p” used herein includes the hsa-miR-135a-3p gene (miRBase Accession No. MIMAT0004595) described in SEQ ID NO: 242, a homolog or an ortholog of a different organism species, and the like. The hsa-miR-135a-3p gene can be obtained by a method described in Lagos-Quintana M et al., 2002, Curr Biol, Vol. 12, p. 735-739. Also, “hsa-mir-135a” (miRBase Accession No. MI0000452, SEQ ID NO: 520) having a hairpin-like structure is known as a precursor of “hsa-miR-135a-3p”.

The term “hsa-miR-486-5p gene” or “hsa-miR-486-5p” used herein includes the hsa-miR-486-5p gene (miRBase Accession No. MIMAT0002177) described in SEQ ID NO: 243, a homolog or an ortholog of a different organism species, and the like. The hsa-miR-486-5p gene can be obtained by a method described in Fu H et al., 2005, FEBS Lett, Vol. 579, p. 3849-3854. Also, “hsa-mir-486 and hsa-mir-486-2” (miRBase Accession Nos. MI0002470 and MI0023622, SEQ ID NOs: 466 and 467) having a hairpin-like structure are known as precursors of “hsa-miR-486-5p”.

The term “hsa-miR-4257 gene” or “hsa-miR-4257” used herein includes the hsa-miR-4257 gene (miRBase Accession No. MIMAT0016878) described in SEQ ID NO: 244, a homolog or an ortholog of a different organism species, and the like. The hsa-miR-4257 gene can be obtained by a method described in Goff L A et al., 2009, PLoS One, Vol. 4, e7192. Also, “hsa-mir-4257” (miRBase Accession No. MI0015856, SEQ ID NO: 521) having a hairpin-like structure is known as a precursor of “hsa-miR-4257”.

The term “hsa-miR-92b-5p gene” or “hsa-miR-92b-5p” used herein includes the hsa-miR-92b-5p gene (miRBase Accession No. MIMAT0004792) described in SEQ ID NO: 245, a homolog or an ortholog of a different organism species, and the like. The hsa-miR-92b-5p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 522) having a hairpin-like structure is known as a precursor of “hsa-miR-92b-5p”.

The term “hsa-miR-1915-3p gene” or “hsa-miR-1915-3p” used herein includes the hsa-miR-1915-3p gene (miRBase Accession No. MIMAT0007892) described in SEQ ID NO: 246, a homolog or an ortholog of a different organism species, and the like. The hsa-miR-1915-3p gene can be obtained by a method described in Bar M et al., 2008, Stem Cells, Vol. 26, p. 2496-2505. Also, “hsa-mir-1915” (miRBase Accession No. MI0008336, SEQ ID NO: 339) having a hairpin-like structure is known as a precursor of “hsa-miR-1915-3p”.

The term “hsa-miR-718 gene” or “hsa-miR-718” used herein includes the hsa-miR-718 gene (miRBase Accession No. MIMAT0012735) described in SEQ ID NO: 247, a homolog or an ortholog of a different organism species, and the like. The hsa-miR-718 gene can be obtained by a method described in Artzi S et al., 2008, BMC Bioinformatics, Vol. 9, p. 39. Also, “hsa-mir-718” (miRBase Accession No. MI0012489, SEQ ID NO: 523) having a hairpin-like structure is known as a precursor of “hsa-miR-718”.

The term “hsa-miR-940 gene” or “hsa-miR-940” used herein includes the hsa-miR-940 gene (miRBase Accession No. MIMAT0004983) described in SEQ ID NO: 248, a homolog or an ortholog of a different organism species, and the like. The hsa-miR-940 gene can be obtained by a method described in Lui W O et al., 2007, Cancer Res, Vol. 67, p. 6031-6043. Also, “hsa-mir-940” (miRBase Accession No. MI0005762, SEQ ID NO: 524) having a hairpin-like structure is known as a precursor of “hsa-miR-940”.

The term “hsa-miR-296-5p gene” or “hsa-miR-296-5p” used herein includes the hsa-miR-296-5p gene (miRBase Accession No. MIMAT0000690) described in SEQ ID NO: 249, a homolog or an ortholog of a different organism species, and the like. The hsa-miR-296-5p gene can be obtained by a method described in Houbaviy H B et al., 2003, Dev Cell, Vol. 5, p. 351-358. Also, “hsa-mir-296” (miRBase Accession No. MI0000747, SEQ ID NO: 409) having a hairpin-like structure is known as a precursor of “hsa-miR-296-5p”.

The term “hsa-miR-23b-3p gene” or “hsa-miR-23b-3p” used herein includes the hsa-miR-23b-3p gene (miRBase Accession No. MIMAT0000418) described in SEQ ID NO: 250, a homolog or an ortholog of a different organism species, and the like. The hsa-miR-23b-3p gene can be obtained by a method described in Lagos-Quintana M et al., 2002, Curr Biol, Vol. 12, p. 735-739. Also, “hsa-mir-23b” (miRBase Accession No. MI0000439, SEQ ID NO: 525) having a hairpin-like structure is known as a precursor of “hsa-miR-23b-3p”.

The term “hsa-miR-92a-3p gene” or “hsa-miR-92a-3p” used herein includes the hsa-miR-92a-3p gene (miRBase Accession No. MIMAT0000092) described in SEQ ID NO: 251, a homolog or an ortholog of a different organism species, and the like. The hsa-miR-92a-3p gene can be obtained by a method described in Mourelatos Z et al., 2002, Genes Dev, Vol. 16, p. 720-728. Also, “hsa-mir-92a-1 and hsa-mir-92a-2” (miRBase Accession Nos. MI0000093 and MI0000094, SEQ ID NOs: 526 and 527) having a hairpin-like structure are known as precursors of “hsa-miR-92a-3p”.

The term “hsa-miR-658 gene” or “hsa-miR-658” used herein includes the hsa-miR-658 gene (miRBase Accession No. MIMAT0003336) described in SEQ ID NO: 252, a homolog or an ortholog of a different organism species, and the like. The hsa-miR-658 gene can be obtained by a method described in Cummins J M et al., 2006, Proc Natl Acad Sci USA, Vol. 103, p. 3687-3692. Also, “hsa-mir-658” (miRBase Accession No. MI0003682, SEQ ID NO: 528) having a hairpin-like structure is known as a precursor of “hsa-miR-658”.

The term “hsa-miR-6842-5p gene” or “hsa-miR-6842-5p” used herein includes the hsa-miR-6842-5p gene (miRBase Accession No. MIMAT0027586) described in SEQ ID NO: 253, a homolog or an ortholog of a different organism species, and the like. The hsa-miR-6842-5p gene can be obtained by a method described in Ladewig E et al., 2012, Genome Res, Vol. 22, p. 1634-1645. Also, “hsa-mir-6842” (miRBase Accession No. MI0022688, SEQ ID NO: 529) having a hairpin-like structure is known as a precursor of “hsa-miR-6842-5p”.

The term “hsa-miR-6124 gene” or “hsa-miR-6124” used herein includes the hsa-miR-6124 gene (miRBase Accession No. MIMAT0024597) described in SEQ ID NO: 254, a homolog or an ortholog of a different organism species, and the like. The hsa-miR-6124 gene can be obtained by a method described in Smith J L et al., 2012, J Virol, Vol. 86, p. 5278-5287. Also, “hsa-mir-6124” (miRBase Accession No. MI0021258, SEQ ID NO: 530) having a hairpin-like structure is known as a precursor of “hsa-miR-6124”.

The term “hsa-miR-6765-3p gene” or “hsa-miR-6765-3p” used herein includes the hsa-miR-6765-3p gene (miRBase Accession No. MIMAT0027431) described in SEQ ID NO: 255, a homolog or an ortholog of a different organism species, and the like. The hsa-miR-6765-3p gene can be obtained by a method described in Ladewig E et al., 2012, Genome Res, Vol. 22, p. 1634-1645. Also, “hsa-mir-6765” (miRBase Accession No. MI0022610, SEQ ID NO: 531) having a hairpin-like structure is known as a precursor of “hsa-miR-6765-3p”.

The term “hsa-miR-7106-5p gene” or “hsa-miR-7106-5p” used herein includes the hsa-miR-7106-5p gene (miRBase Accession No. MIMAT0028109) described in SEQ ID NO: 256, a homolog or an ortholog of a different organism species, and the like. The hsa-miR-7106-5p gene can be obtained by a method described in Ladewig E et al., 2012, Genome Res, Vol. 22, p. 1634-1645. Also, “hsa-mir-7106” (miRBase Accession No. MI0022957, SEQ ID NO: 532) having a hairpin-like structure is known as a precursor of “hsa-miR-7106-5p”.

The term “hsa-miR-4534 gene” or “hsa-miR-4534” used herein includes the hsa-miR-4534 gene (miRBase Accession No. MIMAT0019073) described in SEQ ID NO: 257, a homolog or an ortholog of a different organism species, and the like. The hsa-miR-4534 gene can be obtained by a method described in Jima D D et al., 2010, Blood, Vol. 116, e118-e127. Also, “hsa-mir-4534” (miRBase Accession No. MI0016901, SEQ ID NO: 533) having a hairpin-like structure is known as a precursor of “hsa-miR-4534”.

The term “hsa-miR-92b-3p gene” or “hsa-miR-92b-3p” used herein includes the hsa-miR-92b-3p gene (miRBase Accession No. MIMAT0003218) described in SEQ ID NO: 258, a homolog or an ortholog of a different organism species, and the like. The hsa-miR-92b-3p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 522) having a hairpin-like structure is known as a precursor of “hsa-miR-92b-3p”.

The term “hsa-miR-3135b gene” or “hsa-miR-3135b” used herein includes the hsa-miR-3135b gene (miRBase Accession No. MIMAT0018985) described in SEQ ID NO: 259, a homolog or an ortholog of a different organism species, and the like. The hsa-miR-3135b gene can be obtained by a method described in Jima D D et al., 2010, Blood, Vol. 116, e118-e127. Also, “hsa-mir-3135b” (miRBase Accession No. MI0016809, SEQ ID NO: 534) having a hairpin-like structure is known as a precursor of “hsa-miR-3135b”.

The term “hsa-miR-4687-3p gene” or “hsa-miR-4687-3p” used herein includes the hsa-miR-4687-3p gene (miRBase Accession No. MIMAT0019775) described in SEQ ID NO: 260, a homolog or an ortholog of a different organism species, and the like. The hsa-miR-4687-3p gene can be obtained by a method described in Persson H et al., 2011, Cancer Res, Vol. 71, p. 78-86. Also, “hsa-mir-4687” (miRBase Accession No. MI0017319, SEQ ID NO: 535) having a hairpin-like structure is known as a precursor of “hsa-miR-4687-3p”.

The term “hsa-miR-762 gene” or “hsa-miR-762” used herein includes the hsa-miR-762 gene (miRBase Accession No. MIMAT0010313) described in SEQ ID NO: 261, a homolog or an ortholog of a different organism species, and the like. The hsa-miR-762 gene can be obtained by a method described in Berezikov E et al., 2006, Genome Res, Vol. 16, p. 1289-1298. Also, “hsa-mir-762” (miRBase Accession No. MI0003892, SEQ ID NO: 536) having a hairpin-like structure is known as a precursor of “hsa-miR-762”.

The term “hsa-miR-3619-3p gene” or “hsa-miR-3619-3p” used herein includes the hsa-miR-3619-3p gene (miRBase Accession No. MIMAT0019219) described in SEQ ID NO: 262, a homolog or an ortholog of a different organism species, and the like. The hsa-miR-3619-3p gene can be obtained by a method described in Witten D et al., 2010, BMC Biol, Vol. 8, p. 58. Also, “hsa-mir-3619” (miRBase Accession No. MI0016009, SEQ ID NO: 537) having a hairpin-like structure is known as a precursor of “hsa-miR-3619-3p”.

The term “hsa-miR-4467 gene” or “hsa-miR-4467” used herein includes the hsa-miR-4467 gene (miRBase Accession No. MIMAT0018994) described in SEQ ID NO: 263, a homolog or an ortholog of a different organism species, and the like. The hsa-miR-4467 gene can be obtained by a method described in Jima D D et al., 2010, Blood, Vol. 116, e118-e127. Also, “hsa-mir-4467” (miRBase Accession No. MI0016818, SEQ ID NO: 538) having a hairpin-like structure is known as a precursor of “hsa-miR-4467”.

The term “hsa-miR-557 gene” or “hsa-miR-557” used herein includes the hsa-miR-557 gene (miRBase Accession No. MIMAT0003221) described in SEQ ID NO: 264, a homolog or an ortholog of a different organism species, and the like. The hsa-miR-557 gene can be obtained by a method described in Cummins J M et al., 2006, Proc Natl Acad Sci USA, Vol. 103, p. 3687-3692. Also, “hsa-mir-557” (miRBase Accession No. MI0003563, SEQ ID NO: 539) having a hairpin-like structure is known as a precursor of “hsa-miR-557”.

The term “hsa-miR-1237-5p gene” or “hsa-miR-1237-5p” used herein includes the hsa-miR-1237-5p gene (miRBase Accession No. MIMAT0022946) described in SEQ ID NO: 265, a homolog or an ortholog of a different organism species, and the like. The hsa-miR-1237-5p gene can be obtained by a method described in Berezikov E et al., 2007, Mol Cell, Vol. 28, p. 328-336. Also, “hsa-mir-1237” (miRBase Accession No. MI0006327, SEQ ID NO: 540) having a hairpin-like structure is known as a precursor of “hsa-miR-1237-5p”.

The term “hsa-miR-1908-5p gene” or “hsa-miR-1908-5p” used herein includes the hsa-miR-1908-5p gene (miRBase Accession No. MIMAT0007881) described in SEQ ID NO: 266, a homolog or an ortholog of a different organism species, and the like. The hsa-miR-1908-5p gene can be obtained by a method described in Bar M et al., 2008, Stem Cells, Vol. 26, p. 2496-2505. Also, “hsa-mir-1908” (miRBase Accession No. MI0008329, SEQ ID NO: 541) having a hairpin-like structure is known as a precursor of “hsa-miR-1908-5p”.

The term “hsa-miR-4286 gene” or “hsa-miR-4286” used herein includes the hsa-miR-4286 gene (miRBase Accession No. MIMAT0016916) described in SEQ ID NO: 267, a homolog or an ortholog of a different organism species, and the like. The hsa-miR-4286 gene can be obtained by a method described in Goff L A et al., 2009, PLoS One, Vol. 4, e7192. Also, “hsa-mir-4286” (miRBase Accession No. MI0015894, SEQ ID NO: 542) having a hairpin-like structure is known as a precursor of “hsa-miR-4286”.

The term “hsa-miR-6885-5p gene” or “hsa-miR-6885-5p” used herein includes the hsa-miR-6885-5p gene (miRBase Accession No. MIMAT0027670) described in SEQ ID NO: 268, a homolog or an ortholog of a different organism species, and the like. The hsa-miR-6885-5p gene can be obtained by a method described in Ladewig E et al., 2012, Genome Res, Vol. 22, p. 1634-1645. Also, “hsa-mir-6885” (miRBase Accession No. MI0022732, SEQ ID NO: 543) having a hairpin-like structure is known as a precursor of “hsa-miR-6885-5p”.

The term “hsa-miR-6763-5p gene” or “hsa-miR-6763-5p” used herein includes the hsa-miR-6763-5p gene (miRBase Accession No. MIMAT0027426) described in SEQ ID NO: 269, a homolog or an ortholog of a different organism species, and the like. The hsa-miR-6763-5p gene can be obtained by a method described in Ladewig E et al., 2012, Genome Res, Vol. 22, p. 1634-1645. Also, “hsa-mir-6763” (miRBase Accession No. MI0022608, SEQ ID NO: 544) having a hairpin-like structure is known as a precursor of “hsa-miR-6763-5p”.

The term “hsa-miR-6089 gene” or “hsa-miR-6089” used herein includes the hsa-miR-6089 gene (miRBase Accession No. MIMAT0023714) described in SEQ ID NO: 851, a homolog or an ortholog of a different organism species, and the like. The hsa-miR-6089 gene can be obtained by a method described in Yoo J K et al., 2012, Stem Cells Dev, Vol. 21, p. 2049-2057. Also, “hsa-mir-6089-1 and hsa-mir-6089-2” (miRBase Accession Nos. MI0020366 and MI0023563, SEQ ID NOs: 857 and 858) having a hairpin-like structure are known as precursors of “hsa-miR-6089”.

The term “hsa-miR-6816-5p gene” or “hsa-miR-6816-5p” used herein includes the hsa-miR-6816-5p gene (miRBase Accession No. MIMAT0027532) described in SEQ ID NO: 852, a homolog or an ortholog of a different organism species, and the like. The hsa-miR-6816-5p gene can be obtained by a method described in Ladewig E et al., 2012, Genome Res., Vol. 22, p. 1634-1645. Also, “hsa-mir-6816” (miRBase Accession No. MI0022661, SEQ ID NO: 859) having a hairpin-like structure is known as a precursor of “hsa-miR-6816-5p”.

The term “hsa-miR-4466 gene” or “hsa-miR-4466” used herein includes the hsa-miR-4466 gene (miRBase Accession No. MIMAT0018993) described in SEQ ID NO: 853, a homolog or an ortholog of a different organism species, and the like. The hsa-miR-4466 gene can be obtained by a method described in Jima D D et al., 2010, Blood, Vol. 116, e118-e127. Also, “hsa-mir-4466” (miRBase Accession No. MI0016817, SEQ ID NO: 860) having a hairpin-like structure is known as a precursor of “hsa-miR-4466”.

The term “hsa-miR-4488 gene” or “hsa-miR-4488” used herein includes the hsa-miR-4488 gene (miRBase Accession No. MIMAT0019022) described in SEQ ID NO: 854, a homolog or an ortholog of a different organism species, and the like. The hsa-miR-4488 gene can be obtained by a method described in Jima D D et al., 2010, Blood, Vol. 116, e118-e127. Also, “hsa-mir-4488” (miRBase Accession No. MI0016849, SEQ ID NO: 861) having a hairpin-like structure is known as a precursor of “hsa-miR-4488”.

The term “hsa-miR-6752-5p gene” or “hsa-miR-6752-5p” used herein includes the hsa-miR-6752-5p gene (miRBase Accession No. MIMAT0027404) described in SEQ ID NO: 855, a homolog or an ortholog of a different organism species, and the like. The hsa-miR-6752-5p gene can be obtained by a method described in Ladewig E et al., 2012, Genome Res, Vol. 22, p. 1634-1645. Also, “hsa-mir-6752” (miRBase Accession No. MI0022597, SEQ ID NO: 862) having a hairpin-like structure is known as a precursor of “hsa-miR-6752-5p”.

The term “hsa-miR-4739 gene” or “hsa-miR-4739” used herein includes the hsa-miR-4739 gene (miRBase Accession No. MIMAT0019868) described in SEQ ID NO: 856, a homolog or an ortholog of a different organism species, and the like. The hsa-miR-4739 gene can be obtained by a method described in Persson H et al., 2011, Cancer Res, Vol. 71, p. 78-86. Also, “hsa-mir-4739” (miRBase Accession No. MI0017377, SEQ ID NO: 863) having a hairpin-like structure is known as a precursor of “hsa-miR-4739”.

A mature miRNA may become a variant due to the sequence cleaved shorter or longer by one to several upstream or downstream nucleotide(s) substitution when cleaved as the mature miRNA from its RNA precursor having a hairpin-like structure. This variant is called isomiR (Morin R D. et al., 2008, Genome Res., Vol. 18, p. 610-621). The miRBase Release 20 shows the nucleotide sequences represented by SEQ ID NOs: 1 to 269 and 851 to 856 as well as a large number of the nucleotide sequence variants and fragments represented by SEQ ID NOs: 545 to 850 and 864 to 871, called isomiRs. These variants can also be obtained as miRNAs that have a nucleotide sequence represented by any of SEQ ID NOs: 1 to 269 and 851 to 856. Specifically, among the variants of polynucleotides consisting of a nucleotide sequence represented by any of SEQ ID NOs: 1, 2, 3, 4, 5, 6, 8, 11, 14, 15, 19, 20, 21, 23, 27, 28, 31, 35, 37, 38, 40, 42, 43, 47, 48, 50, 51, 52, 55, 56, 58, 60, 61, 62, 64, 65, 66, 67, 71, 72, 73, 76, 77, 78, 79, 80, 82, 83, 85, 86, 88, 89, 90, 92, 95, 97, 98, 99, 100, 102, 103, 107, 109, 110, 113, 114, 115, 116, 117, 118, 120, 122, 123, 126, 127, 129, 131, 133, 137, 141, 142, 143, 145, 146, 148, 149, 153, 154, 155, 156, 157, 161, 164, 165, 167, 168, 171, 172, 173, 178, 179, 180, 181, 182, 184, 185, 188, 191, 192, 194, 195, 196, 199, 201, 202, 204, 207, 210, 213, 215, 217, 219, 222, 223, 225, 230, 232, 233, 234, 235, 236, 238, 239, 240, 241, 242, 243, 245, 246, 247, 248, 249, 250, 251, 252, 254, 258, 259, 260, 263, 265, 266, 267, 851,853, 854 and 856, or a nucleotide sequence derived from the nucleotide sequence by the replacement of u with t according to the present invention, examples of the longest variants registered in miRBase Release 20 include polynucleotides represented by SEQ ID NOs: 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 864, 866, 868 and 870, respectively. Also, among the variants of polynucleotides consisting of a nucleotide sequence represented by any of SEQ ID NOs: 1, 2, 3, 4, 5, 6, 8, 11, 14, 15, 19, 20, 21, 23, 27, 28, 31, 35, 37, 38, 40, 42, 43, 47, 48, 50, 51, 52, 55, 56, 58, 60, 61, 62, 64, 65, 66, 67, 71, 72, 73, 76, 77, 78, 79, 80, 82, 83, 85, 86, 88, 89, 90, 92, 95, 97, 98, 99, 100, 102, 103, 107, 109, 110, 113, 114, 115, 116, 117, 118, 120, 122, 123, 126, 127, 129, 131, 133, 137, 141, 142, 143, 145, 146, 148, 149, 153, 154, 155, 156, 157, 161, 164, 165, 167, 168, 171, 172, 173, 178, 179, 180, 181, 182, 184, 185, 188, 191, 192, 194, 195, 196, 199, 201, 202, 204, 207, 210, 213, 215, 217, 219, 222, 223, 225, 230, 232, 233, 234, 235, 236, 238, 239, 240, 241, 242, 243, 245, 246, 247, 248, 249, 250, 251, 252, 254, 258, 259, 260, 263, 265, 266, 267, 851,853, 854 and 856, or a nucleotide sequence derived from the nucleotide sequence by the replacement of u with t according to the present invention, examples of the shortest variants registered in miRBase Release 20 include polynucleotides having sequences represented by SEQ ID NOs: 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 865, 867, 869 and 871, respectively. In addition to these variants and fragments, examples thereof include a large number of isomiR polynucleotides of SEQ ID NOs: 1 to 269 and 851 to 856 registered in miRBase. Examples of the polynucleotide comprising a nucleotide sequence represented by any of SEQ ID NOs: 1 to 269 and 851 to 856 include a polynucleotide represented by any of SEQ ID NOs: 270 to 544, and 857 to 863, which are their respective precursors.

The names and miRBase Accession Nos. (registration numbers) of the genes represented by SEQ ID NOs: 1 to 871 are shown in Table 1.

The term “capable of specifically binding” used herein means that the nucleic acid probe or the primer used in the present invention binds to a particular target nucleic acid and cannot substantially bind to other nucleic acids.

TABLE 1

| SEQ | | miRBase |
|---|---|---|
| ID NO: | Gene name | registration No. |
| 1 | hsa-miR-4783-3p | MIMAT0019947 |
| 2 | hsa-miR-4730 | MIMAT0019852 |
| 3 | hsa-miR-1307-3p | MIMAT0005951 |
| 4 | hsa-miR-4634 | MIMAT0019691 |
| 5 | hsa-miR-663a | MIMAT0003326 |
| 6 | hsa-miR-4532 | MIMAT0019071 |
| 7 | hsa-miR-7704 | MIMAT0030019 |
| 8 | hsa-miR-3178 | MIMAT0015055 |
| 9 | hsa-miR-6729-5p | MIMAT0027359 |
| 10 | hsa-miR-6090 | MIMAT0023715 |
| 11 | hsa-miR-4732-5p | MIMAT0019855 |
| 12 | hsa-miR-3184-5p | MIMAT0015064 |
| 13 | hsa-miR-6727-5p | MIMAT0027355 |
| 14 | hsa-miR-6088 | MIMAT0023713 |
| 15 | hsa-miR-4674 | MIMAT0019756 |
| 16 | hsa-miR-8073 | MIMAT0031000 |
| 17 | hsa-miR-4787-5p | MIMAT0019956 |
| 18 | hsa-miR-1469 | MIMAT0007347 |
| 19 | hsa-miR-125a-3p | MIMAT0004602 |
| 20 | hsa-miR-1233-5p | MIMAT0022943 |
| 21 | hsa-miR-885-3p | MIMAT0004948 |
| 22 | hsa-miR-6802-5p | MIMAT0027504 |
| 23 | hsa-miR-328-5p | MIMAT0026486 |
| 24 | hsa-miR-6787-5p | MIMAT0027474 |
| 25 | hsa-miR-8069 | MIMAT0030996 |
| 26 | hsa-miR-6875-5p | MIMAT0027650 |
| 27 | hsa-miR-1246 | MIMAT0005898 |
| 28 | hsa-miR-4734 | MIMAT0019859 |
| 29 | hsa-miR-6757-5p | MIMAT0027414 |
| 30 | hsa-miR-6756-5p | MIMAT0027412 |
| 31 | hsa-miR-3665 | MIMAT0018087 |
| 32 | hsa-miR-6836-3p | MIMAT0027575 |
| 33 | hsa-miR-6821-5p | MIMAT0027542 |
| 34 | hsa-miR-6805-5p | MIMAT0027510 |
| 35 | hsa-miR-4728-5p | MIMAT0019849 |
| 36 | hsa-miR-6726-5p | MIMAT0027353 |
| 37 | hsa-miR-197-5p | MIMAT0022691 |
| 38 | hsa-miR-149-3p | MIMAT0004609 |
| 39 | hsa-miR-6850-5p | MIMAT0027600 |
| 40 | hsa-miR-4476 | MIMAT0019003 |
| 41 | hsa-miR-6858-5p | MIMAT0027616 |
| 42 | hsa-miR-564 | MIMAT0003228 |
| 43 | hsa-miR-4763-3p | MIMAT0019913 |
| 44 | hsa-miR-575 | MIMAT0003240 |
| 45 | hsa-miR-6771-5p | MIMAT0027442 |
| 46 | hsa-miR-1231 | MIMAT0005586 |
| 47 | hsa-miR-1908-3p | MIMAT0026916 |
| 48 | hsa-miR-150-3p | MIMAT0004610 |
| 49 | hsa-miR-3937 | MIMAT0018352 |
| 50 | hsa-miR-887-3p | MIMAT0004951 |
| 51 | hsa-miR-3940-5p | MIMAT0019229 |
| 52 | hsa-miR-4741 | MIMAT0019871 |
| 53 | hsa-miR-6808-5p | MIMAT0027516 |
| 54 | hsa-miR-6869-5p | MIMAT0027638 |
| 55 | hsa-miR-5090 | MIMAT0021082 |
| 56 | hsa-miR-615-5p | MIMAT0004804 |
| 57 | hsa-miR-8072 | MIMAT0030999 |
| 58 | hsa-miR-128-1-5p | MIMAT0026477 |
| 59 | hsa-miR-1238-5p | MIMAT0022947 |
| 60 | hsa-miR-365a-5p | MIMAT0009199 |
| 61 | hsa-miR-204-3p | MIMAT0022693 |
| 62 | hsa-miR-4492 | MIMAT0019027 |
| 63 | hsa-miR-6785-5p | MIMAT0027470 |
| 64 | hsa-miR-6511a-5p | MIMAT0025478 |
| 65 | hsa-miR-4525 | MIMAT0019064 |
| 66 | hsa-miR-1915-5p | MIMAT0007891 |
| 67 | hsa-miR-3180 | MIMAT0018178 |
| 68 | hsa-miR-6879-5p | MIMAT0027658 |
| 69 | hsa-miR-1199-5p | MIMAT0031119 |
| 70 | hsa-miR-6746-5p | MIMAT0027392 |
| 71 | hsa-miR-711 | MIMAT0012734 |
| 72 | hsa-miR-663b | MIMAT0005867 |
| 73 | hsa-miR-4707-3p | MIMAT0019808 |
| 74 | hsa-miR-6893-5p | MIMAT0027686 |
| 75 | hsa-miR-4675 | MIMAT0019757 |
| 76 | hsa-miR-4638-5p | MIMAT0019695 |
| 77 | hsa-miR-4651 | MIMAT0019715 |
| 78 | hsa-miR-6087 | MIMAT0023712 |
| 79 | hsa-miR-4665-5p | MIMAT0019739 |
| 80 | hsa-miR-4758-5p | MIMAT0019903 |
| 81 | hsa-miR-6887-5p | MIMAT0027674 |
| 82 | hsa-miR-3620-5p | MIMAT0022967 |
| 83 | hsa-miR-1909-3p | MIMAT0007883 |
| 84 | hsa-miR-7641 | MIMAT0029782 |
| 85 | hsa-miR-6724-5p | MIMAT0025856 |
| 86 | hsa-miR-1343-3p | MIMAT0019776 |
| 87 | hsa-miR-6780b-5p | MIMAT0027572 |
| 88 | hsa-miR-4484 | MIMAT0019018 |
| 89 | hsa-miR-4690-5p | MIMAT0019779 |
| 90 | hsa-miR-4429 | MIMAT0018944 |
| 91 | hsa-miR-1227-5p | MIMAT0022941 |
| 92 | hsa-miR-4725-3p | MIMAT0019844 |
| 93 | hsa-miR-6861-5p | MIMAT0027623 |
| 94 | hsa-miR-6812-5p | MIMAT0027524 |
| 95 | hsa-miR-3197 | MIMAT0015082 |
| 96 | hsa-miR-8059 | MIMAT0030986 |
| 97 | hsa-miR-3185 | MIMAT0015065 |
| 98 | hsa-miR-4706 | MIMAT0019806 |
| 99 | hsa-miR-4497 | MIMAT0019032 |
| 100 | hsa-miR-3131 | MIMAT0014996 |
| 101 | hsa-miR-6806-5p | MIMAT0027512 |
| 102 | hsa-miR-187-5p | MIMAT0004561 |
| 103 | hsa-miR-3180-3p | MIMAT0015058 |
| 104 | hsa-miR-6848-5p | MIMAT0027596 |
| 105 | hsa-miR-6820-5p | MIMAT0027540 |
| 106 | hsa-miR-6800-5p | MIMAT0027500 |
| 107 | hsa-miR-6717-5p | MIMAT0025846 |
| 108 | hsa-miR-6795-5p | MIMAT0027490 |
| 109 | hsa-miR-4632-5p | MIMAT0022977 |
| 110 | hsa-miR-665 | MIMAT0004952 |
| 111 | hsa-miR-6778-5p | MIMAT0027456 |
| 112 | hsa-miR-3663-3p | MIMAT0018085 |
| 113 | hsa-miR-4689 | MIMAT0019778 |
| 114 | hsa-miR-211-3p | MIMAT0022694 |
| 115 | hsa-miR-6511b-5p | MIMAT0025847 |
| 116 | hsa-miR-4750-5p | MIMAT0019887 |
| 117 | hsa-miR-6126 | MIMAT0024599 |
| 118 | hsa-miR-614 | MIMAT0003282 |
| 119 | hsa-miR-7110-5p | MIMAT0028117 |
| 120 | hsa-miR-744-5p | MIMAT0004945 |
| 121 | hsa-miR-6769a-5p | MIMAT0027438 |
| 122 | hsa-miR-4792 | MIMAT0019964 |
| 123 | hsa-miR-5787 | MIMAT0023252 |
| 124 | hsa-miR-6798-5p | MIMAT0027496 |
| 125 | hsa-miR-6781-5p | MIMAT0027462 |
| 126 | hsa-miR-4419b | MIMAT0019034 |
| 127 | hsa-miR-4446-3p | MIMAT0018965 |
| 128 | hsa-miR-4259 | MIMAT0016880 |
| 129 | hsa-miR-5572 | MIMAT0022260 |
| 130 | hsa-miR-6075 | MIMAT0023700 |
| 131 | hsa-miR-296-3p | MIMAT0004679 |
| 132 | hsa-miR-6891-5p | MIMAT0027682 |
| 133 | hsa-miR-4745-5p | MIMAT0019878 |
| 134 | hsa-miR-6775-5p | MIMAT0027450 |
| 135 | hsa-miR-6870-5p | MIMAT0027640 |
| 136 | hsa-miR-920 | MIMAT0004970 |
| 137 | hsa-miR-4530 | MIMAT0019069 |
| 138 | hsa-miR-6819-5p | MIMAT0027538 |
| 139 | hsa-miR-6825-5p | MIMAT0027550 |
| 140 | hsa-miR-7847-3p | MIMAT0030422 |
| 141 | hsa-miR-6131 | MIMAT0024615 |
| 142 | hsa-miR-4433-3p | MIMAT0018949 |
| 143 | hsa-miR-1228-5p | MIMAT0005582 |
| 144 | hsa-miR-6743-5p | MIMAT0027387 |
| 145 | hsa-miR-1268a | MIMAT0005922 |
| 146 | hsa-miR-3917 | MIMAT0018191 |
| 147 | hsa-miR-6786-5p | MIMAT0027472 |
| 148 | hsa-miR-3154 | MIMAT0015028 |
| 149 | hsa-miR-638 | MIMAT0003308 |
| 150 | hsa-miR-6741-5p | MIMAT0027383 |
| 151 | hsa-miR-6889-5p | MIMAT0027678 |
| 152 | hsa-miR-6840-3p | MIMAT0027583 |
| 153 | hsa-miR-6510-5p | MIMAT0025476 |
| 154 | hsa-miR-3188 | MIMAT0015070 |
| 155 | hsa-miR-551b-5p | MIMAT0004794 |
| 156 | hsa-miR-5001-5p | MIMAT0021021 |
| 157 | hsa-miR-1268b | MIMAT0018925 |
| 158 | hsa-miR-7107-5p | MIMAT0028111 |
| 159 | hsa-miR-6824-5p | MIMAT0027548 |
| 160 | hsa-miR-6732-5p | MIMAT0027365 |
| 161 | hsa-miR-371a-5p | MIMAT0004687 |
| 162 | hsa-miR-6794-5p | MIMAT0027488 |
| 163 | hsa-miR-6779-5p | MIMAT0027458 |
| 164 | hsa-miR-4271 | MIMAT0016901 |
| 165 | hsa-miR-5195-3p | MIMAT0021127 |
| 166 | hsa-miR-6762-5p | MIMAT0027424 |
| 167 | hsa-miR-939-5p | MIMAT0004982 |
| 168 | hsa-miR-1247-3p | MIMAT0022721 |
| 169 | hsa-miR-6777-5p | MIMAT0027454 |
| 170 | hsa-miR-6722-3p | MIMAT0025854 |
| 171 | hsa-miR-3656 | MIMAT0018076 |
| 172 | hsa-miR-4688 | MIMAT0019777 |
| 173 | hsa-miR-3195 | MIMAT0015079 |
| 174 | hsa-miR-6766-5p | MIMAT0027432 |
| 175 | hsa-miR-4447 | MIMAT0018966 |
| 176 | hsa-miR-4656 | MIMAT0019723 |
| 177 | hsa-miR-7108-5p | MIMAT0028113 |
| 178 | hsa-miR-3191-3p | MIMAT0015075 |
| 179 | hsa-miR-1273g-3p | MIMAT0022742 |
| 180 | hsa-miR-4463 | MIMAT0018987 |
| 181 | hsa-miR-2861 | MIMAT0013802 |
| 182 | hsa-miR-3196 | MIMAT0015080 |
| 183 | hsa-miR-6877-5p | MIMAT0027654 |
| 184 | hsa-miR-3679-5p | MIMAT0018104 |
| 185 | hsa-miR-4442 | MIMAT0018960 |
| 186 | hsa-miR-6789-5p | MIMAT0027478 |
| 187 | hsa-miR-6782-5p | MIMAT0027464 |
| 188 | hsa-miR-486-3p | MIMAT0004762 |
| 189 | hsa-miR-6085 | MIMAT0023710 |
| 190 | hsa-miR-4746-3p | MIMAT0019881 |
| 191 | hsa-miR-619-5p | MIMAT0026622 |
| 192 | hsa-miR-937-5p | MIMAT0022938 |
| 193 | hsa-miR-6803-5p | MIMAT0027506 |
| 194 | hsa-miR-4298 | MIMAT0016852 |
| 195 | hsa-miR-4454 | MIMAT0018976 |
| 196 | hsa-miR-4459 | MIMAT0018981 |
| 197 | hsa-miR-7150 | MIMAT0028211 |
| 198 | hsa-miR-6880-5p | MIMAT0027660 |
| 199 | hsa-miR-4449 | MIMAT0018968 |
| 200 | hsa-miR-8063 | MIMAT0030990 |
| 201 | hsa-miR-4695-5p | MIMAT0019788 |
| 202 | hsa-miR-6132 | MIMAT0024616 |
| 203 | hsa-miR-6829-5p | MIMAT0027558 |
| 204 | hsa-miR-4486 | MIMAT0019020 |
| 205 | hsa-miR-6805-3p | MIMAT0027511 |
| 206 | hsa-miR-6826-5p | MIMAT0027552 |
| 207 | hsa-miR-4508 | MIMAT0019045 |
| 208 | hsa-miR-1343-5p | MIMAT0027038 |
| 209 | hsa-miR-7114-5p | MIMAT0028125 |
| 210 | hsa-miR-3622a-5p | MIMAT0018003 |
| 211 | hsa-miR-6765-5p | MIMAT0027430 |
| 212 | hsa-miR-7845-5p | MIMAT0030420 |
| 213 | hsa-miR-3960 | MIMAT0019337 |
| 214 | hsa-miR-6749-5p | MIMAT0027398 |
| 215 | hsa-miR-1260b | MIMAT0015041 |
| 216 | hsa-miR-6799-5p | MIMAT0027498 |
| 217 | hsa-miR-4723-5p | MIMAT0019838 |
| 218 | hsa-miR-6784-5p | MIMAT0027468 |
| 219 | hsa-miR-5100 | MIMAT0022259 |
| 220 | hsa-miR-6769b-5p | MIMAT0027620 |
| 221 | hsa-miR-1207-5p | MIMAT0005871 |
| 222 | hsa-miR-642a-3p | MIMAT0020924 |
| 223 | hsa-miR-4505 | MIMAT0019041 |
| 224 | hsa-miR-4270 | MIMAT0016900 |
| 225 | hsa-miR-6721-5p | MIMAT0025852 |
| 226 | hsa-miR-7111-5p | MIMAT0028119 |
| 227 | hsa-miR-6791-5p | MIMAT0027482 |
| 228 | hsa-miR-7109-5p | MIMAT0028115 |
| 229 | hsa-miR-4258 | MIMAT0016879 |
| 230 | hsa-miR-6515-3p | MIMAT0025487 |
| 231 | hsa-miR-6851-5p | MIMAT0027602 |
| 232 | hsa-miR-6125 | MIMAT0024598 |
| 233 | hsa-miR-4749-5p | MIMAT0019885 |
| 234 | hsa-miR-4726-5p | MIMAT0019845 |
| 235 | hsa-miR-4513 | MIMAT0019050 |
| 236 | hsa-miR-760 | MIMAT0004957 |
| 237 | hsa-miR-602 | MIMAT0003270 |
| 238 | hsa-miR-423-5p | MIMAT0004748 |
| 239 | hsa-miR-92a-2-5p | MIMAT0004508 |
| 240 | hsa-miR-16-5p | MIMAT0000069 |
| 241 | hsa-miR-451a | MIMAT0001631 |
| 242 | hsa-miR-135a-3p | MIMAT0004595 |
| 243 | hsa-miR-486-5p | MIMAT0002177 |
| 244 | hsa-miR-4257 | MIMAT0016878 |
| 245 | hsa-miR-92b-5p | MIMAT0004792 |
| 246 | hsa-miR-1915-3p | MIMAT0007892 |
| 247 | hsa-miR-718 | MIMAT0012735 |
| 248 | hsa-miR-940 | MIMAT0004983 |
| 249 | hsa-miR-296-5p | MIMAT0000690 |
| 250 | hsa-miR-23b-3p | MIMAT0000418 |
| 251 | hsa-miR-92a-3p | MIMAT0000092 |
| 252 | hsa-miR-658 | MIMAT0003336 |
| 253 | hsa-miR-6842-5p | MIMAT0027586 |
| 254 | hsa-miR-6124 | MIMAT0024597 |
| 255 | hsa-miR-6765-3p | MIMAT0027431 |
| 256 | hsa-miR-7106-5p | MIMAT0028109 |
| 257 | hsa-miR-4534 | MIMAT0019073 |
| 258 | hsa-miR-92b-3p | MIMAT0003218 |
| 259 | hsa-miR-3135b | MIMAT0018985 |
| 260 | hsa-miR-4687-3p | MIMAT0019775 |
| 261 | hsa-miR-762 | MIMAT0010313 |
| 262 | hsa-miR-3619-3p | MIMAT0019219 |
| 263 | hsa-miR-4467 | MIMAT0018994 |
| 264 | hsa-miR-557 | MIMAT0003221 |
| 265 | hsa-miR-1237-5p | MIMAT0022946 |
| 266 | hsa-miR-1908-5p | MIMAT0007881 |
| 267 | hsa-miR-4286 | MIMAT0016916 |
| 268 | hsa-miR-6885-5p | MIMAT0027670 |
| 269 | hsa-miR-6763-5p | MIMAT0027426 |
| 270 | hsa-mir-4783 | MI0017428 |
| 271 | hsa-mir-4730 | MI0017367 |
| 272 | hsa-mir-1307 | MI0006444 |
| 273 | hsa-mir-4634 | MI0017261 |
| 274 | hsa-mir-663a | MI0003672 |
| 275 | hsa-mir-4532 | MI0016899 |
| 276 | hsa-mir-7704 | MI0025240 |
| 277 | hsa-mir-3178 | MI0014212 |
| 278 | hsa-mir-6729 | MI0022574 |
| 279 | hsa-mir-6090 | MI0020367 |
| 280 | hsa-mir-4732 | MI0017369 |
| 281 | hsa-mir-3184 | MI0014226 |
| 282 | hsa-mir-6727 | MI0022572 |
| 283 | hsa-mir-6088 | MI0020365 |
| 284 | hsa-mir-4674 | MI0017305 |
| 285 | hsa-mir-8073 | MI0025909 |
| 286 | hsa-mir-4787 | MI0017434 |
| 287 | hsa-mir-1469 | MI0007074 |
| 288 | hsa-mir-125a | MI0000469 |
| 289 | hsa-mir-1233-1 | MI0006323 |
| 290 | hsa-mir-1233-2 | MI0015973 |
| 291 | hsa-mir-885 | MI0005560 |
| 292 | hsa-mir-6802 | MI0022647 |
| 293 | hsa-mir-328 | MI0000804 |
| 294 | hsa-mir-6787 | MI0022632 |
| 295 | hsa-mir-8069 | MI0025905 |
| 296 | hsa-mir-6875 | MI0022722 |
| 297 | hsa-mir-1246 | MI0006381 |
| 298 | hsa-mir-4734 | MI0017371 |
| 299 | hsa-mir-6757 | MI0022602 |
| 300 | hsa-mir-6756 | MI0022601 |
| 301 | hsa-mir-3665 | MI0016066 |
| 302 | hsa-mir-6836 | MI0022682 |
| 303 | hsa-mir-6821 | MI0022666 |
| 304 | hsa-mir-6805 | MI0022650 |
| 305 | hsa-mir-4728 | MI0017365 |
| 306 | hsa-mir-6726 | MI0022571 |
| 307 | hsa-mir-197 | MI0000239 |
| 308 | hsa-mir-149 | MI0000478 |
| 309 | hsa-mir-6850 | MI0022696 |
| 310 | hsa-mir-4476 | MI0016828 |
| 311 | hsa-mir-6858 | MI0022704 |
| 312 | hsa-mir-564 | MI0003570 |
| 313 | hsa-mir-4763 | MI0017404 |
| 314 | hsa-mir-575 | MI0003582 |
| 315 | hsa-mir-6771 | MI0022616 |
| 316 | hsa-mir-1231 | MI0006321 |
| 317 | hsa-mir-1908 | MI0008329 |
| 318 | hsa-mir-150 | MI0000479 |
| 319 | hsa-mir-3937 | MI0016593 |
| 320 | hsa-mir-887 | MI0005562 |
| 321 | hsa-mir-3940 | MI0016597 |
| 322 | hsa-mir-4741 | MI0017379 |
| 323 | hsa-mir-6808 | MI0022653 |
| 324 | hsa-mir-6869 | MI0022716 |
| 325 | hsa-mir-5090 | MI0017979 |
| 326 | hsa-mir-615 | MI0003628 |
| 327 | hsa-mir-8072 | MI0025908 |
| 328 | hsa-mir-128-1 | MI0000447 |
| 329 | hsa-mir-1238 | MI0006328 |
| 330 | hsa-mir-365a | MI0000767 |
| 331 | hsa-mir-204 | MI0000284 |
| 332 | hsa-mir-4492 | MI0016854 |
| 333 | hsa-mir-6785 | MI0022630 |
| 334 | hsa-mir-6511a-1 | MI0022223 |
| 335 | hsa-mir-6511a-2 | MI0023564 |
| 336 | hsa-mir-6511a-3 | MI0023565 |
| 337 | hsa-mir-6511a-4 | MI0023566 |
| 338 | hsa-mir-4525 | MI0016892 |
| 339 | hsa-mir-1915 | MI0008336 |
| 340 | hsa-mir-3180-4 | MI0016408 |
| 341 | hsa-mir-3180-5 | MI0016409 |
| 342 | hsa-mir-6879 | MI0022726 |
| 343 | hsa-mir-1199 | MI0020340 |
| 344 | hsa-mir-6746 | MI0022591 |
| 345 | hsa-mir-711 | MI0012488 |
| 346 | hsa-mir-663b | MI0006336 |
| 347 | hsa-mir-4707 | MI0017340 |
| 348 | hsa-mir-6893 | MI0022740 |
| 349 | hsa-mir-4675 | MI0017306 |
| 350 | hsa-mir-4638 | MI0017265 |
| 351 | hsa-mir-4651 | MI0017279 |
| 352 | hsa-mir-6087 | MI0020364 |
| 353 | hsa-mir-4665 | MI0017295 |
| 354 | hsa-mir-4758 | MI0017399 |
| 355 | hsa-mir-6887 | MI0022734 |
| 356 | hsa-mir-3620 | MI0016011 |
| 357 | hsa-mir-1909 | MI0008330 |
| 358 | hsa-mir-7641-1 | MI0024975 |
| 359 | hsa-mir-7641-2 | MI0024976 |
| 360 | hsa-mir-6724 | MI0022559 |
| 361 | hsa-mir-1343 | MI0017320 |
| 362 | hsa-mir-6780b | MI0022681 |
| 363 | hsa-mir-4484 | MI0016845 |
| 364 | hsa-mir-4690 | MI0017323 |
| 365 | hsa-mir-4429 | MI0016768 |
| 366 | hsa-mir-1227 | MI0006316 |
| 367 | hsa-mir-4725 | MI0017362 |
| 368 | hsa-mir-6861 | MI0022708 |
| 369 | hsa-mir-6812 | MI0022657 |
| 370 | hsa-mir-3197 | MI0014245 |
| 371 | hsa-mir-8059 | MI0025895 |
| 372 | hsa-mir-3185 | MI0014227 |
| 373 | hsa-mir-4706 | MI0017339 |
| 374 | hsa-mir-4497 | MI0016859 |
| 375 | hsa-mir-3131 | MI0014151 |
| 376 | hsa-mir-6806 | MI0022651 |
| 377 | hsa-mir-187 | MI0000274 |
| 378 | hsa-mir-3180-1 | MI0014214 |
| 379 | hsa-mir-3180-2 | MI0014215 |
| 380 | hsa-mir-3180-3 | MI0014217 |
| 381 | hsa-mir-6848 | MI0022694 |
| 382 | hsa-mir-6820 | MI0022665 |
| 383 | hsa-mir-6800 | MI0022645 |
| 384 | hsa-mir-6717 | MI0022551 |
| 385 | hsa-mir-6795 | MI0022640 |
| 386 | hsa-mir-4632 | MI0017259 |
| 387 | hsa-mir-665 | MI0005563 |
| 388 | hsa-mir-6778 | MI0022623 |
| 389 | hsa-mir-3663 | MI0016064 |
| 390 | hsa-mir-4689 | MI0017322 |
| 391 | hsa-mir-211 | MI0000287 |
| 392 | hsa-mir-6511b-1 | MI0022552 |
| 393 | hsa-mir-6511b-2 | MI0023431 |
| 394 | hsa-mir-4750 | MI0017389 |
| 395 | hsa-mir-6126 | MI0021260 |
| 396 | hsa-mir-614 | MI0003627 |
| 397 | hsa-mir-7110 | MI0022961 |
| 398 | hsa-mir-744 | MI0005559 |
| 399 | hsa-mir-6769a | MI0022614 |
| 400 | hsa-mir-4792 | MI0017439 |
| 401 | hsa-mir-5787 | MI0019797 |
| 402 | hsa-mir-6798 | MI0022643 |
| 403 | hsa-mir-6781 | MI0022626 |
| 404 | hsa-mir-4419b | MI0016861 |
| 405 | hsa-mir-4446 | MI0016789 |
| 406 | hsa-mir-4259 | MI0015858 |
| 407 | hsa-mir-5572 | MI0019117 |
| 408 | hsa-mir-6075 | MI0020352 |
| 409 | hsa-mir-296 | MI0000747 |
| 410 | hsa-mir-6891 | MI0022738 |
| 411 | hsa-mir-4745 | MI0017384 |
| 412 | hsa-mir-6775 | MI0022620 |
| 413 | hsa-mir-6870 | MI0022717 |
| 414 | hsa-mir-920 | MI0005712 |
| 415 | hsa-mir-4530 | MI0016897 |
| 416 | hsa-mir-6819 | MI0022664 |
| 417 | hsa-mir-6825 | MI0022670 |
| 418 | hsa-mir-7847 | MI0025517 |
| 419 | hsa-mir-6131 | MI0021276 |
| 420 | hsa-mir-4433 | MI0016773 |
| 421 | hsa-mir-1228 | MI0006318 |
| 422 | hsa-mir-6743 | MI0022588 |
| 423 | hsa-mir-1268a | MI0006405 |
| 424 | hsa-mir-3917 | MI0016423 |
| 425 | hsa-mir-6786 | MI0022631 |
| 426 | hsa-mir-3154 | MI0014182 |
| 427 | hsa-mir-638 | MI0003653 |
| 428 | hsa-mir-6741 | MI0022586 |
| 429 | hsa-mir-6889 | MI0022736 |
| 430 | hsa-mir-6840 | MI0022686 |
| 431 | hsa-mir-6510 | MI0022222 |
| 432 | hsa-mir-3188 | MI0014232 |
| 433 | hsa-mir-551b | MI0003575 |
| 434 | hsa-mir-5001 | MI0017867 |
| 435 | hsa-mir-1268b | MI0016748 |
| 436 | hsa-mir-7107 | MI0022958 |
| 437 | hsa-mir-6824 | MI0022669 |
| 438 | hsa-mir-6732 | MI0022577 |
| 439 | hsa-mir-371a | MI0000779 |
| 440 | hsa-mir-6794 | MI0022639 |
| 441 | hsa-mir-6779 | MI0022624 |
| 442 | hsa-mir-4271 | MI0015879 |
| 443 | hsa-mir-5195 | MI0018174 |
| 444 | hsa-mir-6762 | MI0022607 |
| 445 | hsa-mir-939 | MI0005761 |
| 446 | hsa-mir-1247 | MI0006382 |
| 447 | hsa-mir-6777 | MI0022622 |
| 448 | hsa-mir-6722 | MI0022557 |
| 449 | hsa-mir-3656 | MI0016056 |
| 450 | hsa-mir-4688 | MI0017321 |
| 451 | hsa-mir-3195 | MI0014240 |
| 452 | hsa-mir-6766 | MI0022611 |
| 453 | hsa-mir-4447 | MI0016790 |
| 454 | hsa-mir-4656 | MI0017284 |
| 455 | hsa-mir-7108 | MI0022959 |
| 456 | hsa-mir-3191 | MI0014236 |
| 457 | hsa-mir-1273g | MI0018003 |
| 458 | hsa-mir-4463 | MI0016811 |
| 459 | hsa-mir-2861 | MI0013006 |
| 460 | hsa-mir-3196 | MI0014241 |
| 461 | hsa-mir-6877 | MI0022724 |
| 462 | hsa-mir-3679 | MI0016080 |
| 463 | hsa-mir-4442 | MI0016785 |
| 464 | hsa-mir-6789 | MI0022634 |
| 465 | hsa-mir-6782 | MI0022627 |
| 466 | hsa-mir-486 | MI0002470 |
| 467 | hsa-mir-486-2 | MI0023622 |
| 468 | hsa-mir-6085 | MI0020362 |
| 469 | hsa-mir-4746 | MI0017385 |
| 470 | hsa-mir-619 | MI0003633 |
| 471 | hsa-mir-937 | MI0005759 |
| 472 | hsa-mir-6803 | MI0022648 |
| 473 | hsa-mir-4298 | MI0015830 |
| 474 | hsa-mir-4454 | MI0016800 |
| 475 | hsa-mir-4459 | MI0016805 |
| 476 | hsa-mir-7150 | MI0023610 |
| 477 | hsa-mir-6880 | MI0022727 |
| 478 | hsa-mir-4449 | MI0016792 |
| 479 | hsa-mir-8063 | MI0025899 |
| 480 | hsa-mir-4695 | MI0017328 |
| 481 | hsa-mir-6132 | MI0021277 |
| 482 | hsa-mir-6829 | MI0022674 |
| 483 | hsa-mir-4486 | MI0016847 |
| 484 | hsa-mir-6826 | MI0022671 |
| 485 | hsa-mir-4508 | MI0016872 |
| 486 | hsa-mir-7114 | MI0022965 |
| 487 | hsa-mir-3622a | MI0016013 |
| 488 | hsa-mir-6765 | MI0022610 |
| 489 | hsa-mir-7845 | MI0025515 |
| 490 | hsa-mir-3960 | MI0016964 |
| 491 | hsa-mir-6749 | MI0022594 |
| 492 | hsa-mir-1260b | MI0014197 |
| 493 | hsa-mir-6799 | MI0022644 |
| 494 | hsa-mir-4723 | MI0017359 |
| 495 | hsa-mir-6784 | MI0022629 |
| 496 | hsa-mir-5100 | MI0019116 |
| 497 | hsa-mir-6769b | MI0022706 |
| 498 | hsa-mir-1207 | MI0006340 |
| 499 | hsa-mir-642a | MI0003657 |
| 500 | hsa-mir-4505 | MI0016868 |
| 501 | hsa-mir-4270 | MI0015878 |
| 502 | hsa-mir-6721 | MI0022556 |
| 503 | hsa-mir-7111 | MI0022962 |
| 504 | hsa-mir-6791 | MI0022636 |
| 505 | hsa-mir-7109 | MI0022960 |
| 506 | hsa-mir-4258 | MI0015857 |
| 507 | hsa-mir-6515 | MI0022227 |
| 508 | hsa-mir-6851 | MI0022697 |
| 509 | hsa-mir-6125 | MI0021259 |
| 510 | hsa-mir-4749 | MI0017388 |
| 511 | hsa-mir-4726 | MI0017363 |
| 512 | hsa-mir-4513 | MI0016879 |
| 513 | hsa-mir-760 | MI0005567 |
| 514 | hsa-mir-602 | MI0003615 |
| 515 | hsa-mir-423 | MI0001445 |
| 516 | hsa-mir-92a-2 | MI0000094 |
| 517 | hsa-mir-16-1 | MI0000070 |
| 518 | hsa-mir-16-2 | MI0000115 |
| 519 | hsa-mir-451a | MI0001729 |
| 520 | hsa-mir-135a | MI0000452 |
| 521 | hsa-mir-4257 | MI0015856 |
| 522 | hsa-mir-92b | MI0003560 |
| 523 | hsa-mir-718 | MI0012489 |
| 524 | hsa-mir-940 | MI0005762 |
| 525 | hsa-mir-23b | MI0000439 |
| 526 | hsa-mir-92a-1 | MI0000093 |
| 527 | hsa-mir-92a-2 | MI0000094 |
| 528 | hsa-mir-658 | MI0003682 |
| 529 | hsa-mir-6842 | MI0022688 |
| 530 | hsa-mir-6124 | MI0021258 |
| 531 | hsa-mir-6765 | MI0022610 |
| 532 | hsa-mir-7106 | MI0022957 |
| 533 | hsa-mir-4534 | MI0016901 |
| 534 | hsa-mir-3135b | MI0016809 |
| 535 | hsa-mir-4687 | MI0017319 |
| 536 | hsa-mir-762 | MI0003892 |
| 537 | hsa-mir-3619 | MI0016009 |
| 538 | hsa-mir-4467 | MI0016818 |
| 539 | hsa-mir-557 | MI0003563 |
| 540 | hsa-mir-1237 | MI0006327 |
| 541 | hsa-mir-1908 | MI0008329 |
| 542 | hsa-mir-4286 | MI0015894 |
| 543 | hsa-mir-6885 | MI0022732 |
| 544 | hsa-mir-6763 | MI0022608 |
| 545 | isomiR example 1 of SEQ ID NO: 1 | — |
| 546 | isomiR example 2 of SEQ ID NO: 1 | — |
| 547 | isomiR example 1 of SEQ ID NO: 2 | — |
| 548 | isomiR example 2 of SEQ ID NO: 2 | — |
| 549 | isomiR example 1 of SEQ ID NO: 3 | — |
| 550 | isomiR example 2 of SEQ ID NO: 3 | — |
| 551 | isomiR example 1 of SEQ ID NO: 4 | — |
| 552 | isomiR example 2 of SEQ ID NO: 4 | — |
| 553 | isomiR example 1 of SEQ ID NO: 5 | — |
| 554 | isomiR example 2 of SEQ ID NO: 5 | — |
| 555 | isomiR example 1 of SEQ ID NO: 6 | — |
| 556 | isomiR example 2 of SEQ ID NO: 6 | — |
| 557 | isomiR example 1 of SEQ ID NO: 8 | — |
| 558 | isomiR example 2 of SEQ ID NO: 8 | — |
| 559 | isomiR example 1 of SEQ ID NO: 11 | — |
| 560 | isomiR example 2 of SEQ ID NO: 11 | — |
| 561 | isomiR example 1 of SEQ ID NO: 14 | — |
| 562 | isomiR example 2 of SEQ ID NO: 14 | — |
| 563 | isomiR example 1 of SEQ ID NO: 15 | — |
| 564 | isomiR example 2 of SEQ ID NO: 15 | — |
| 565 | isomiR example 1 of SEQ ID NO: 19 | — |
| 566 | isomiR example 2 of SEQ ID NO: 19 | — |
| 567 | isomiR example 1 of SEQ ID NO: 20 | — |
| 568 | isomiR example 2 of SEQ ID NO: 20 | — |
| 569 | isomiR example 1 of SEQ ID NO: 21 | — |
| 570 | isomiR example 2 of SEQ ID NO: 21 | — |
| 571 | isomiR example 1 of SEQ ID NO: 23 | — |
| 572 | isomiR example 2 of SEQ ID NO: 23 | — |
| 573 | isomiR example 1 of SEQ ID NO: 27 | — |
| 574 | isomiR example 2 of SEQ ID NO: 27 | — |
| 575 | isomiR example 1 of SEQ ID NO: 28 | — |
| 576 | isomiR example 2 of SEQ ID NO: 28 | — |
| 577 | isomiR example 1 of SEQ ID NO: 31 | — |
| 578 | isomiR example 2 of SEQ ID NO: 31 | — |
| 579 | isomiR example 1 of SEQ ID NO: 35 | — |
| 580 | isomiR example 2 of SEQ ID NO: 35 | — |
| 581 | isomiR example 1 of SEQ ID NO: 37 | — |
| 582 | isomiR example 2 of SEQ ID NO: 37 | — |
| 583 | isomiR example 1 of SEQ ID NO: 38 | — |
| 584 | isomiR example 2 of SEQ ID NO: 38 | — |
| 585 | isomiR example 1 of SEQ ID NO: 40 | — |
| 586 | isomiR example 2 of SEQ ID NO: 40 | — |
| 587 | isomiR example 1 of SEQ ID NO: 42 | — |
| 588 | isomiR example 2 of SEQ ID NO: 42 | — |
| 589 | isomiR example 1 of SEQ ID NO: 43 | — |
| 590 | isomiR example 2 of SEQ ID NO: 43 | — |
| 591 | isomiR example 1 of SEQ ID NO: 47 | — |
| 592 | isomiR example 2 of SEQ ID NO: 47 | — |
| 593 | isomiR example 1 of SEQ ID NO: 48 | — |
| 594 | isomiR example 2 of SEQ ID NO: 48 | — |
| 595 | isomiR example 1 of SEQ ID NO: 50 | — |
| 596 | isomiR example 2 of SEQ ID NO: 50 | — |
| 597 | isomiR example 1 of SEQ ID NO: 51 | — |
| 598 | isomiR example 2 of SEQ ID NO: 51 | — |
| 599 | isomiR example 1 of SEQ ID NO: 52 | — |
| 600 | isomiR example 2 of SEQ ID NO: 52 | — |
| 601 | isomiR example 1 of SEQ ID NO: 55 | — |
| 602 | isomiR example 2 of SEQ ID NO: 55 | — |
| 603 | isomiR example 1 of SEQ ID NO: 56 | — |
| 604 | isomiR example 2 of SEQ ID NO: 56 | — |
| 605 | isomiR example 1 of SEQ ID NO: 58 | — |
| 606 | isomiR example 2 of SEQ ID NO: 58 | — |
| 607 | isomiR example 1 of SEQ ID NO: 60 | — |
| 608 | isomiR example 2 of SEQ ID NO: 60 | — |
| 609 | isomiR example 1 of SEQ ID NO: 61 | — |
| 610 | isomiR example 2 of SEQ ID NO: 61 | — |
| 611 | isomiR example 1 of SEQ ID NO: 62 | — |
| 612 | isomiR example 2 of SEQ ID NO: 62 | — |
| 613 | isomiR example 1 of SEQ ID NO: 64 | — |
| 614 | isomiR example 2 of SEQ ID NO: 64 | — |
| 615 | isomiR example 1 of SEQ ID NO: 65 | — |
| 616 | isomiR example 2 of SEQ ID NO: 65 | — |
| 617 | isomiR example 1 of SEQ ID NO: 66 | — |
| 618 | isomiR example 2 of SEQ ID NO: 66 | — |
| 619 | isomiR example 1 of SEQ ID NO: 67 | — |
| 620 | isomiR example 2 of SEQ ID NO: 67 | — |
| 621 | isomiR example 1 of SEQ ID NO: 71 | — |
| 622 | isomiR example 2 of SEQ ID NO: 71 | — |
| 623 | isomiR example 1 of SEQ ID NO: 72 | — |
| 624 | isomiR example 2 of SEQ ID NO: 72 | — |
| 625 | isomiR example 1 of SEQ ID NO: 73 | — |
| 626 | isomiR example 2 of SEQ ID NO: 73 | — |
| 627 | isomiR example 1 of SEQ ID NO: 76 | — |
| 628 | isomiR example 2 of SEQ ID NO: 76 | — |
| 629 | isomiR example 1 of SEQ ID NO: 77 | — |
| 630 | isomiR example 2 of SEQ ID NO: 77 | — |
| 631 | isomiR example 1 of SEQ ID NO: 78 | — |
| 632 | isomiR example 2 of SEQ ID NO: 78 | — |
| 633 | isomiR example 1 of SEQ ID NO: 79 | — |
| 634 | isomiR example 2 of SEQ ID NO: 79 | — |
| 635 | isomiR example 1 of SEQ ID NO: 80 | — |
| 636 | isomiR example 2 of SEQ ID NO: 80 | — |
| 637 | isomiR example 1 of SEQ ID NO: 82 | — |
| 638 | isomiR example 2 of SEQ ID NO: 82 | — |
| 639 | isomiR example 1 of SEQ ID NO: 83 | — |
| 640 | isomiR example 2 of SEQ ID NO: 83 | — |
| 641 | isomiR example 1 of SEQ ID NO: 85 | — |
| 642 | isomiR example 2 of SEQ ID NO: 85 | — |
| 643 | isomiR example 1 of SEQ ID NO: 86 | — |
| 644 | isomiR example 2 of SEQ ID NO: 86 | — |
| 645 | isomiR example 1 of SEQ ID NO: 88 | — |
| 646 | isomiR example 2 of SEQ ID NO: 88 | — |
| 647 | isomiR example 1 of SEQ ID NO: 89 | — |
| 648 | isomiR example 2 of SEQ ID NO: 89 | — |
| 649 | isomiR example 1 of SEQ ID NO: 90 | — |
| 650 | isomiR example 2 of SEQ ID NO: 90 | — |
| 651 | isomiR example 1 of SEQ ID NO: 92 | — |
| 652 | isomiR example 2 of SEQ ID NO: 92 | — |
| 653 | isomiR example 1 of SEQ ID NO: 95 | — |
| 654 | isomiR example 2 of SEQ ID NO: 95 | — |
| 655 | isomiR example 1 of SEQ ID NO: 97 | — |
| 656 | isomiR example 2 of SEQ ID NO: 97 | — |
| 657 | isomiR example 1 of SEQ ID NO: 98 | — |
| 658 | isomiR example 2 of SEQ ID NO: 98 | — |
| 659 | isomiR example 1 of SEQ ID NO: 99 | — |
| 660 | isomiR example 2 of SEQ ID NO: 99 | — |
| 661 | isomiR example 1 of SEQ ID NO: 100 | — |
| 662 | isomiR example 2 of SEQ ID NO: 100 | — |
| 663 | isomiR example 1 of SEQ ID NO: 102 | — |
| 664 | isomiR example 2 of SEQ ID NO: 102 | — |
| 665 | isomiR example 1 of SEQ ID NO: 103 | — |
| 666 | isomiR example 2 of SEQ ID NO: 103 | — |
| 667 | isomiR example 1 of SEQ ID NO: 107 | — |
| 668 | isomiR example 2 of SEQ ID NO: 107 | — |
| 669 | isomiR example 1 of SEQ ID NO: 109 | — |
| 670 | isomiR example 2 of SEQ ID NO: 109 | — |
| 671 | isomiR example 1 of SEQ ID NO: 110 | — |
| 672 | isomiR example 2 of SEQ ID NO: 110 | — |
| 673 | isomiR example 1 of SEQ ID NO: 113 | — |
| 674 | isomiR example 2 of SEQ ID NO: 113 | — |
| 675 | isomiR example 1 of SEQ ID NO: 114 | — |
| 676 | isomiR example 2 of SEQ ID NO: 114 | — |
| 677 | isomiR example 1 of SEQ ID NO: 115 | — |
| 678 | isomiR example 2 of SEQ ID NO: 115 | — |
| 679 | isomiR example 1 of SEQ ID NO: 116 | — |
| 680 | isomiR example 2 of SEQ ID NO: 116 | — |
| 681 | isomiR example 1 of SEQ ID NO: 117 | — |
| 682 | isomiR example 2 of SEQ ID NO: 117 | — |
| 683 | isomiR example 1 of SEQ ID NO: 118 | — |
| 684 | isomiR example 2 of SEQ ID NO: 118 | — |
| 685 | isomiR example 1 of SEQ ID NO: 120 | — |
| 686 | isomiR example 2 of SEQ ID NO: 120 | — |
| 687 | isomiR example 1 of SEQ ID NO: 122 | — |
| 688 | isomiR example 2 of SEQ ID NO: 122 | — |
| 689 | isomiR example 1 of SEQ ID NO: 123 | — |
| 690 | isomiR example 2 of SEQ ID NO: 123 | — |
| 691 | isomiR example 1 of SEQ ID NO: 126 | — |
| 692 | isomiR example 2 of SEQ ID NO: 126 | — |
| 693 | isomiR example 1 of SEQ ID NO: 127 | — |
| 694 | isomiR example 2 of SEQ ID NO: 127 | — |
| 695 | isomiR example 1 of SEQ ID NO: 129 | — |
| 696 | isomiR example 2 of SEQ ID NO: 129 | — |
| 697 | isomiR example 1 of SEQ ID NO: 131 | — |
| 698 | isomiR example 2 of SEQ ID NO: 131 | — |
| 699 | isomiR example 1 of SEQ ID NO: 133 | — |
| 700 | isomiR example 2 of SEQ ID NO: 133 | — |
| 701 | isomiR example 1 of SEQ ID NO: 137 | — |
| 702 | isomiR example 2 of SEQ ID NO: 137 | — |
| 703 | isomiR example 1 of SEQ ID NO: 141 | — |
| 704 | isomiR example 2 of SEQ ID NO: 141 | — |
| 705 | isomiR example 1 of SEQ ID NO: 142 | — |
| 706 | isomiR example 2 of SEQ ID NO: 142 | — |
| 707 | isomiR example 1 of SEQ ID NO: 143 | — |
| 708 | isomiR example 2 of SEQ ID NO: 143 | — |
| 709 | isomiR example 1 of SEQ ID NO: 145 | — |
| 710 | isomiR example 2 of SEQ ID NO: 145 | — |
| 711 | isomiR example 1 of SEQ ID NO: 146 | — |
| 712 | isomiR example 2 of SEQ ID NO: 146 | — |
| 713 | isomiR example 1 of SEQ ID NO: 148 | — |
| 714 | isomiR example 2 of SEQ ID NO: 148 | — |
| 715 | isomiR example 1 of SEQ ID NO: 149 | — |
| 716 | isomiR example 2 of SEQ ID NO: 149 | — |
| 717 | isomiR example 1 of SEQ ID NO: 153 | — |
| 718 | isomiR example 2 of SEQ ID NO: 153 | — |
| 719 | isomiR example 1 of SEQ ID NO: 154 | — |
| 720 | isomiR example 2 of SEQ ID NO: 154 | — |
| 721 | isomiR example 1 of SEQ ID NO: 155 | — |
| 722 | isomiR example 2 of SEQ ID NO: 155 | — |
| 723 | isomiR example 1 of SEQ ID NO: 156 | — |
| 724 | isomiR example 2 of SEQ ID NO: 156 | — |
| 725 | isomiR example 1 of SEQ ID NO: 157 | — |
| 726 | isomiR example 2 of SEQ ID NO: 157 | — |
| 727 | isomiR example 1 of SEQ ID NO: 161 | — |
| 728 | isomiR example 2 of SEQ ID NO: 161 | — |
| 729 | isomiR example 1 of SEQ ID NO: 164 | — |
| 730 | isomiR example 2 of SEQ ID NO: 164 | — |
| 731 | isomiR example 1 of SEQ ID NO: 165 | — |
| 732 | isomiR example 2 of SEQ ID NO: 165 | — |
| 733 | isomiR example 1 of SEQ ID NO: 167 | — |
| 734 | isomiR example 2 of SEQ ID NO: 167 | — |
| 735 | isomiR example 1 of SEQ ID NO: 168 | — |
| 736 | isomiR example 2 of SEQ ID NO: 168 | — |
| 737 | isomiR example 1 of SEQ ID NO: 171 | — |
| 738 | isomiR example 2 of SEQ ID NO: 171 | — |
| 739 | isomiR example 1 of SEQ ID NO: 172 | — |
| 740 | isomiR example 2 of SEQ ID NO: 172 | — |
| 741 | isomiR example 1 of SEQ ID NO: 173 | — |
| 742 | isomiR example 2 of SEQ ID NO: 173 | — |
| 743 | isomiR example 1 of SEQ ID NO: 178 | — |
| 744 | isomiR example 2 of SEQ ID NO: 178 | — |
| 745 | isomiR example 1 of SEQ ID NO: 179 | — |
| 746 | isomiR example 2 of SEQ ID NO: 179 | — |
| 747 | isomiR example 1 of SEQ ID NO: 180 | — |
| 748 | isomiR example 2 of SEQ ID NO: 180 | — |
| 749 | isomiR example 1 of SEQ ID NO: 181 | — |
| 750 | isomiR example 2 of SEQ ID NO: 181 | — |
| 751 | isomiR example 1 of SEQ ID NO: 182 | — |
| 752 | isomiR example 2 of SEQ ID NO: 182 | — |
| 753 | isomiR example 1 of SEQ ID NO: 184 | — |
| 754 | isomiR example 2 of SEQ ID NO: 184 | — |
| 755 | isomiR example 1 of SEQ ID NO: 185 | — |
| 756 | isomiR example 2 of SEQ ID NO: 185 | — |
| 757 | isomiR example 1 of SEQ ID NO: 188 | — |
| 758 | isomiR example 2 of SEQ ID NO: 188 | — |
| 759 | isomiR example 1 of SEQ ID NO: 191 | — |
| 760 | isomiR example 2 of SEQ ID NO: 191 | — |
| 761 | isomiR example 1 of SEQ ID NO: 192 | — |
| 762 | isomiR example 2 of SEQ ID NO: 192 | — |
| 763 | isomiR example 1 of SEQ ID NO: 194 | — |
| 764 | isomiR example 2 of SEQ ID NO: 194 | — |
| 765 | isomiR example 1 of SEQ ID NO: 195 | — |
| 766 | isomiR example 2 of SEQ ID NO: 195 | — |
| 767 | isomiR example 1 of SEQ ID NO: 196 | — |
| 768 | isomiR example 2 of SEQ ID NO: 196 | — |
| 769 | isomiR example 1 of SEQ ID NO: 199 | — |
| 770 | isomiR example 2 of SEQ ID NO: 199 | — |
| 771 | isomiR example 1 of SEQ ID NO: 201 | — |
| 772 | isomiR example 2 of SEQ ID NO: 201 | — |
| 773 | isomiR example 1 of SEQ ID NO: 202 | — |
| 774 | isomiR example 2 of SEQ ID NO: 202 | — |
| 775 | isomiR example 1 of SEQ ID NO: 204 | — |
| 776 | isomiR example 2 of SEQ ID NO: 204 | — |
| 777 | isomiR example 1 of SEQ ID NO: 207 | — |
| 778 | isomiR example 2 of SEQ ID NO: 207 | — |
| 779 | isomiR example 1 of SEQ ID NO: 210 | — |
| 780 | isomiR example 2 of SEQ ID NO: 210 | — |
| 781 | isomiR example 1 of SEQ ID NO: 213 | — |
| 782 | isomiR example 2 of SEQ ID NO: 213 | — |
| 783 | isomiR example 1 of SEQ ID NO: 215 | — |
| 784 | isomiR example 2 of SEQ ID NO: 215 | — |
| 785 | isomiR example 1 of SEQ ID NO: 217 | — |
| 786 | isomiR example 2 of SEQ ID NO: 217 | — |
| 787 | isomiR example 1 of SEQ ID NO: 219 | — |
| 788 | isomiR example 2 of SEQ ID NO: 219 | — |
| 789 | isomiR example 1 of SEQ ID NO: 222 | — |
| 790 | isomiR example 2 of SEQ ID NO: 222 | — |
| 791 | isomiR example 1 of SEQ ID NO: 223 | — |
| 792 | isomiR example 2 of SEQ ID NO: 223 | — |
| 793 | isomiR example 1 of SEQ ID NO: 225 | — |
| 794 | isomiR example 2 of SEQ ID NO: 225 | — |
| 795 | isomiR example 1 of SEQ ID NO: 230 | — |
| 796 | isomiR example 2 of SEQ ID NO: 230 | — |
| 797 | isomiR example 1 of SEQ ID NO: 232 | — |
| 798 | isomiR example 2 of SEQ ID NO: 232 | — |
| 799 | isomiR example 1 of SEQ ID NO: 233 | — |
| 800 | isomiR example 2 of SEQ ID NO: 233 | — |
| 801 | isomiR example 1 of SEQ ID NO: 234 | — |
| 802 | isomiR example 2 of SEQ ID NO: 234 | — |
| 803 | isomiR example 1 of SEQ ID NO: 235 | — |
| 804 | isomiR example 2 of SEQ ID NO: 235 | — |
| 805 | isomiR example 1 of SEQ ID NO: 236 | — |
| 806 | isomiR example 2 of SEQ ID NO: 236 | — |
| 807 | isomiR example 1 of SEQ ID NO: 238 | — |
| 808 | isomiR example 2 of SEQ ID NO: 238 | — |
| 809 | isomiR example 1 of SEQ ID NO: 239 | — |
| 810 | isomiR example 2 of SEQ ID NO: 239 | — |
| 811 | isomiR example 1 of SEQ ID NO: 240 | — |
| 812 | isomiR example 2 of SEQ ID NO: 240 | — |
| 813 | isomiR example 1 of SEQ ID NO: 241 | — |
| 814 | isomiR example 2 of SEQ ID NO: 241 | — |
| 815 | isomiR example 1 of SEQ ID NO: 242 | — |
| 816 | isomiR example 2 of SEQ ID NO: 242 | — |
| 817 | isomiR example 1 of SEQ ID NO: 243 | — |
| 818 | isomiR example 2 of SEQ ID NO: 243 | — |
| 819 | isomiR example 1 of SEQ ID NO: 245 | — |
| 820 | isomiR example 2 of SEQ ID NO: 245 | — |
| 821 | isomiR example 1 of SEQ ID NO: 246 | — |
| 822 | isomiR example 2 of SEQ ID NO: 246 | — |
| 823 | isomiR example 1 of SEQ ID NO: 247 | — |
| 824 | isomiR example 2 of SEQ ID NO: 247 | — |
| 825 | isomiR example 1 of SEQ ID NO: 248 | — |
| 826 | isomiR example 2 of SEQ ID NO: 248 | — |
| 827 | isomiR example 1 of SEQ ID NO: 249 | — |
| 828 | isomiR example 2 of SEQ ID NO: 249 | — |
| 829 | isomiR example 1 of SEQ ID NO: 250 | — |
| 830 | isomiR example 2 of SEQ ID NO: 250 | — |
| 831 | isomiR example 1 of SEQ ID NO: 251 | — |
| 832 | isomiR example 2 of SEQ ID NO: 251 | — |
| 833 | isomiR example 1 of SEQ ID NO: 252 | — |
| 834 | isomiR example 2 of SEQ ID NO: 252 | — |
| 835 | isomiR example 1 of SEQ ID NO: 254 | — |
| 836 | isomiR example 2 of SEQ ID NO: 254 | — |
| 837 | isomiR example 1 of SEQ ID NO: 258 | — |
| 838 | isomiR example 2 of SEQ ID NO: 258 | — |
| 839 | isomiR example 1 of SEQ ID NO: 259 | — |
| 840 | isomiR example 2 of SEQ ID NO: 259 | — |
| 841 | isomiR example 1 of SEQ ID NO: 260 | — |
| 842 | isomiR example 2 of SEQ ID NO: 260 | — |
| 843 | isomiR example 1 of SEQ ID NO: 263 | — |
| 844 | isomiR example 2 of SEQ ID NO: 263 | — |
| 845 | isomiR example 1 of SEQ ID NO: 265 | — |
| 846 | isomiR example 2 of SEQ ID NO: 265 | — |
| 847 | isomiR example 1 of SEQ ID NO: 266 | — |
| 848 | isomiR example 2 of SEQ ID NO: 266 | — |
| 849 | isomiR example 1 of SEQ ID NO: 267 | — |
| 850 | isomiR example 2 of SEQ ID NO: 267 | — |
| 851 | hsa-miR-6089 | MIMAT0023714 |
| 852 | hsa-miR-6816-5p | MIMAT0027532 |
| 853 | hsa-miR-4466 | MIMAT0018993 |
| 854 | hsa-miR-4488 | MIMAT0019022 |
| 855 | hsa-miR-6752-5p | MIMAT0027404 |
| 856 | hsa-miR-4739 | MIMAT0019868 |
| 857 | hsa-mir-6089-1 | MI0020366 |
| 858 | hsa-mir-6089-2 | MI0023563 |
| 859 | hsa-mir-6816 | MI0022661 |
| 860 | hsa-mir-4466 | MI0016817 |
| 861 | hsa-mir-4488 | MI0016849 |
| 862 | hsa-mir-6752 | MI0022597 |
| 863 | hsa-mir-4739 | MI0017377 |
| 864 | isomiR example 1 of SEQ ID NO: 851 | — |
| 865 | isomiR example 2 of SEQ ID NO: 851 | — |
| 866 | isomiR example 1 of SEQ ID NO: 853 | — |
| 867 | isomiR example 2 of SEQ ID NO: 853 | — |
| 868 | isomiR example 1 of SEQ ID NO: 854 | — |
| 869 | isomiR example 2 of SEQ ID NO: 854 | — |
| 870 | isomiR example 1 of SEQ ID NO: 856 | — |
| 871 | isomiR example 2 of SEQ ID NO: 856 | — |

The present specification encompasses the contents described in the specifications and/or drawings of Japanese Patent Application No. 2014-122672 and No. 2015-069321 from which the present application claims priorities.

### Advantageous Effect of Invention

According to the present invention, breast cancer can be detected easily and in high accuracy. For example, the presence or absence of breast cancer in patients can be easily detected by using, as indicators, the measurement values of several miRNAs in bloods, sera, and/or plasmas of the patients, which can be collected with limitedly invasiveness.

### BRIEF DESCRIPTION OF DRAWINGS

FIG. 1 This figure shows the relationship between the nucleotide sequences of hsa-miR-6805-5p represented by SEQ ID NO: 34 and hsa-miR-6805-3p represented by SEQ ID NO: 205, which are produced from a precursor hsa-mir-6805 represented by SEQ ID NO: 304.

FIG. 2 Left diagram: the expression level measurement values of hsa-miR-4783-3p (SEQ ID NO: 1) in healthy subjects (100 persons) and breast cancer patients (62 persons) selected as a training cohort were each plotted on the ordinate. The horizontal line in the diagram depicts a threshold (6.63) that was optimized by Fisher's discriminant analysis and discriminated between the two groups. Right diagram: the expression level measurement values of hsa-miR-4783-3p (SEQ ID NO: 1) in healthy subjects (50 persons) and breast cancer patients (31 persons) selected as a validation cohort were each plotted on the ordinate. The horizontal line in the diagram depicts the threshold (6.63) that was set in the training cohort and discriminated between the two groups.

FIG. 3 Left diagram: the expression level measurement values of hsa-miR-4783-3p (SEQ ID NO: 1) in healthy subjects (100 persons, circles) and breast cancer patients (62 persons, triangles) selected as a training cohort were each plotted on the abscissa against their expression level measurement values of hsa-miR-4730 (SEQ ID NO: 2) on the ordinate. The line in the diagram depicts a discriminant function (0=1.41x+y+0.77) that was optimized by Fisher's discriminant analysis and discriminated between the two groups. Right diagram: the expression level measurement values of hsa-miR-4783-3p (SEQ ID NO: 1) in healthy subjects (50 persons, circles) and breast cancer patients (31 persons, triangles) selected as a validation cohort were each plotted on the abscissa against their expression level measurement values of hsa-miR-4730 (SEQ ID NO: 2) on the ordinate. The line in the diagram depicts the threshold (0=1.41x+y+0.77) that was set for the training cohort and discriminated between the two groups.

FIG. 4 Upper diagram: a discriminant (1.87×hsa-miR-4730+0.42×hsa-miR-602-18.58) was prepared by use of Fisher's discriminant analysis from the expression level measurement values of hsa-miR-602 (SEQ ID NO: 237) and hsa-miR-4730 (SEQ ID NO: 2) in 62 breast cancer patients, 102 healthy subjects, and 33 prostate cancer patients selected as a training cohort, and discriminant scores obtained from the discriminant were plotted on the ordinate against the sample groups on the abscissa. The dotted line in the diagram depicts a discriminant boundary that offered a discriminant score of 0 and discriminated between the groups. Lower diagram: discriminant scores obtained from the discriminant prepared for the training cohort as to the expression level measurement values of hsa-miR-602 (SEQ ID NO: 237) and hsa-miR-4730 (SEQ ID NO: 2) in 31 breast cancer patients, 48 healthy subjects, and 19 prostate cancer patients selected as a validation cohort were plotted on the ordinate against the sample groups on the abscissa. The dotted line in the diagram depicts the discriminant boundary that offered a discriminant score of 0 and discriminated between the two groups.

### DESCRIPTION OF EMBODIMENTS

Hereinafter, the present invention will be further described in detail.

1. Target Nucleic Acid for Breast Cancer

Primary target nucleic acids as breast cancer markers for detecting the presence and/or absence of breast cancer or breast cancer cells using the nucleic acid probes or the primers for the detection of breast cancer defined above according to the present invention, at least one miRNA selected from the group consisting of the following miRNAs: hsa-miR-4783-3p, hsa-miR-4730, hsa-miR-1307-3p, hsa-miR-4634, hsa-miR-663a, hsa-miR-4532, hsa-miR-7704, hsa-miR-3178, hsa-miR-6729-5p, hsa-miR-6090, hsa-miR-4732-5p, hsa-miR-3184-5p, hsa-miR-6727-5p, hsa-miR-6088, hsa-miR-4674, hsa-miR-8073, hsa-miR-4787-5p, hsa-miR-1469, hsa-miR-125a-3p, hsa-miR-1233-5p, hsa-miR-885-3p, hsa-miR-6802-5p, hsa-miR-328-5p, hsa-miR-6787-5p, hsa-miR-8069, hsa-miR-6875-5p, hsa-miR-1246, hsa-miR-4734, hsa-miR-6757-5p, hsa-miR-6756-5p, hsa-miR-3665, hsa-miR-6836-3p, hsa-miR-6821-5p, hsa-miR-6805-5p, hsa-miR-4728-5p, hsa-miR-6726-5p, hsa-miR-197-5p, hsa-miR-149-3p, hsa-miR-6850-5p, hsa-miR-4476, hsa-miR-6858-5p, hsa-miR-564, hsa-miR-4763-3p, hsa-miR-575, hsa-miR-6771-5p, hsa-miR-1231, hsa-miR-1908-3p, hsa-miR-150-3p, hsa-miR-3937, hsa-miR-887-3p, hsa-miR-3940-5p, hsa-miR-4741, hsa-miR-6808-5p, hsa-miR-6869-5p, hsa-miR-5090, hsa-miR-615-5p, hsa-miR-8072, hsa-miR-128-1-5p, hsa-miR-1238-5p, hsa-miR-365a-5p, hsa-miR-204-3p, hsa-miR-4492, hsa-miR-6785-5p, hsa-miR-6511a-5p, hsa-miR-4525, hsa-miR-1915-5p, hsa-miR-3180, hsa-miR-6879-5p, hsa-miR-1199-5p, hsa-miR-6746-5p, hsa-miR-711, hsa-miR-663b, hsa-miR-4707-3p, hsa-miR-6893-5p, hsa-miR-4675, hsa-miR-4638-5p, hsa-miR-4651, hsa-miR-6087, hsa-miR-4665-5p, hsa-miR-4758-5p, hsa-miR-6887-5p, hsa-miR-3620-5p, hsa-miR-1909-3p, hsa-miR-7641, hsa-miR-6724-5p, hsa-miR-1343-3p, hsa-miR-6780b-5p, hsa-miR-4484, hsa-miR-4690-5p, hsa-miR-4429, hsa-miR-1227-5p, hsa-miR-4725-3p, hsa-miR-6861-5p, hsa-miR-6812-5p, hsa-miR-3197, hsa-miR-8059, hsa-miR-3185, hsa-miR-4706, hsa-miR-4497, hsa-miR-3131, hsa-miR-6806-5p, hsa-miR-187-5p, hsa-miR-3180-3p, hsa-miR-6848-5p, hsa-miR-6820-5p, hsa-miR-6800-5p, hsa-miR-6717-5p, hsa-miR-6795-5p, hsa-miR-4632-5p, hsa-miR-665, hsa-miR-6778-5p, hsa-miR-3663-3p, hsa-miR-4689, hsa-miR-211-3p, hsa-miR-6511b-5p, hsa-miR-4750-5p, hsa-miR-6126, hsa-miR-614, hsa-miR-7110-5p, hsa-miR-744-5p, hsa-miR-6769a-5p, hsa-miR-4792, hsa-miR-5787, hsa-miR-6798-5p, hsa-miR-6781-5p, hsa-miR-4419b, hsa-miR-4446-3p, hsa-miR-4259, hsa-miR-5572, hsa-miR-6075, hsa-miR-296-3p, hsa-miR-6891-5p, hsa-miR-4745-5p, hsa-miR-6775-5p, hsa-miR-6870-5p, hsa-miR-920, hsa-miR-4530, hsa-miR-6819-5p, hsa-miR-6825-5p, hsa-miR-7847-3p, hsa-miR-6131, hsa-miR-4433-3p, hsa-miR-1228-5p, hsa-miR-6743-5p, hsa-miR-1268a, hsa-miR-3917, hsa-miR-6786-5p, hsa-miR-3154, hsa-miR-638, hsa-miR-6741-5p, hsa-miR-6889-5p, hsa-miR-6840-3p, hsa-miR-6510-5p, hsa-miR-3188,hsa-miR-551b-5p, hsa-miR-5001-5p, hsa-miR-1268b, hsa-miR-7107-5p, hsa-miR-6824-5p, hsa-miR-6732-5p, hsa-miR-371a-5p, hsa-miR-6794-5p, hsa-miR-6779-5p, hsa-miR-4271, hsa-miR-5195-3p, hsa-miR-6762-5p, hsa-miR-939-5p, hsa-miR-1247-3p, hsa-miR-6777-5p, hsa-miR-6722-3p, hsa-miR-3656, hsa-miR-4688, hsa-miR-3195, hsa-miR-6766-5p, hsa-miR-4447, hsa-miR-4656, hsa-miR-7108-5p, hsa-miR-3191-3p, hsa-miR-1273g-3p, hsa-miR-4463, hsa-miR-2861, hsa-miR-3196, hsa-miR-6877-5p, hsa-miR-3679-5p, hsa-miR-4442, hsa-miR-6789-5p, hsa-miR-6782-5p, hsa-miR-486-3p, hsa-miR-6085, hsa-miR-4746-3p, hsa-miR-619-5p, hsa-miR-937-5p, hsa-miR-6803-5p, hsa-miR-4298, hsa-miR-4454, hsa-miR-4459, hsa-miR-7150, hsa-miR-6880-5p, hsa-miR-4449, hsa-miR-8063, hsa-miR-4695-5p, hsa-miR-6132, hsa-miR-6829-5p, hsa-miR-4486, hsa-miR-6805-3p, hsa-miR-6826-5p, hsa-miR-4508, hsa-miR-1343-5p, hsa-miR-7114-5p, hsa-miR-3622a-5p, hsa-miR-6765-5p, hsa-miR-7845-5p, hsa-miR-3960, hsa-miR-6749-5p, hsa-miR-1260b, hsa-miR-6799-5p, hsa-miR-4723-5p, hsa-miR-6784-5p, hsa-miR-5100, hsa-miR-6769b-5p, hsa-miR-1207-5p, hsa-miR-642a-3p, hsa-miR-4505, hsa-miR-4270, hsa-miR-6721-5p, hsa-miR-7111-5p, hsa-miR-6791-5p, hsa-miR-7109-5p, hsa-miR-4258, hsa-miR-6515-3p, hsa-miR-6851-5p, hsa-miR-6125, hsa-miR-4749-5p, hsa-miR-4726-5p, hsa-miR-4513, hsa-miR-6089, hsa-miR-6816-5p, hsa-miR-4466, hsa-miR-4488, hsa-miR-6752-5p and hsa-miR-4739 can be used. Furthermore, at least one miRNA selected from the group consisting of the following other breast cancer markers that can be combined with these miRNAs, i.e., hsa-miR-760, hsa-miR-602, hsa-miR-423-5p, hsa-miR-92a-2-5p, hsa-miR-16-5p, hsa-miR-451a, hsa-miR-135a-3p, hsa-miR-486-5p, hsa-miR-4257, hsa-miR-92b-5p, hsa-miR-1915-3p, hsa-miR-718, hsa-miR-940, hsa-miR-296-5p, hsa-miR-23b-3p and hsa-miR-92a-3p can also be preferably used as a target nucleic acid. Moreover, at least one miRNA selected from the group consisting of the following other breast cancer markers that can be combined with these miRNAs, i.e., hsa-miR-658, hsa-miR-6842-5p, hsa-miR-6124, hsa-miR-6765-3p, hsa-miR-7106-5p, hsa-miR-4534, hsa-miR-92b-3p, hsa-miR-3135b, hsa-miR-4687-3p, hsa-miR-762, hsa-miR-3619-3p, hsa-miR-4467, hsa-miR-557, hsa-miR-1237-5p, hsa-miR-1908-5p, hsa-miR-4286, hsa-miR-6885-5p and hsa-miR-6763-5p can also be preferably used as target nucleic acids.

These miRNAs include, for example, a human gene comprising a nucleotide sequence represented by any of SEQ ID NOs: 1 to 269, and 851 to 856 (i.e., hsa-miR-4783-3p, hsa-miR-4730, hsa-miR-1307-3p, hsa-miR-4634, hsa-miR-663a, hsa-miR-4532, hsa-miR-7704, hsa-miR-3178, hsa-miR-6729-5p, hsa-miR-6090, hsa-miR-4732-5p, hsa-miR-3184-5p, hsa-miR-6727-5p, hsa-miR-6088, hsa-miR-4674, hsa-miR-8073, hsa-miR-4787-5p, hsa-miR-1469, hsa-miR-125a-3p, hsa-miR-1233-5p, hsa-miR-885-3p, hsa-miR-6802-5p, hsa-miR-328-5p, hsa-miR-6787-5p, hsa-miR-8069, hsa-miR-6875-5p, hsa-miR-1246, hsa-miR-4734, hsa-miR-6757-5p, hsa-miR-6756-5p, hsa-miR-3665, hsa-miR-6836-3p, hsa-miR-6821-5p, hsa-miR-6805-5p, hsa-miR-4728-5p, hsa-miR-6726-5p, hsa-miR-197-5p, hsa-miR-149-3p, hsa-miR-6850-5p, hsa-miR-4476, hsa-miR-6858-5p, hsa-miR-564, hsa-miR-4763-3p, hsa-miR-575, hsa-miR-6771-5p, hsa-miR-1231, hsa-miR-1908-3p, hsa-miR-150-3p, hsa-miR-3937, hsa-miR-887-3p, hsa-miR-3940-5p, hsa-miR-4741, hsa-miR-6808-5p, hsa-miR-6869-5p, hsa-miR-5090, hsa-miR-615-5p, hsa-miR-8072, hsa-miR-128-1-5p, hsa-miR-1238-5p, hsa-miR-365a-5p, hsa-miR-204-3p, hsa-miR-4492, hsa-miR-6785-5p, hsa-miR-6511a-5p, hsa-miR-4525, hsa-miR-1915-5p, hsa-miR-3180, hsa-miR-6879-5p, hsa-miR-1199-5p, hsa-miR-6746-5p, hsa-miR-711, hsa-miR-663b, hsa-miR-4707-3p, hsa-miR-6893-5p, hsa-miR-4675, hsa-miR-4638-5p, hsa-miR-4651, hsa-miR-6087, hsa-miR-4665-5p, hsa-miR-4758-5p, hsa-miR-6887-5p, hsa-miR-3620-5p, hsa-miR-1909-3p, hsa-miR-7641, hsa-miR-6724-5p, hsa-miR-1343-3p, hsa-miR-6780b-5p, hsa-miR-4484, hsa-miR-4690-5p, hsa-miR-4429, hsa-miR-1227-5p, hsa-miR-4725-3p, hsa-miR-6861-5p, hsa-miR-6812-5p, hsa-miR-3197, hsa-miR-8059, hsa-miR-3185, hsa-miR-4706, hsa-miR-4497, hsa-miR-3131, hsa-miR-6806-5p, hsa-miR-187-5p, hsa-miR-3180-3p, hsa-miR-6848-5p, hsa-miR-6820-5p, hsa-miR-6800-5p, hsa-miR-6717-5p, hsa-miR-6795-5p, hsa-miR-4632-5p, hsa-miR-665, hsa-miR-6778-5p, hsa-miR-3663-3p, hsa-miR-4689, hsa-miR-211-3p, hsa-miR-6511b-5p, hsa-miR-4750-5p, hsa-miR-6126, hsa-miR-614, hsa-miR-7110-5p, hsa-miR-744-5p, hsa-miR-6769a-5p, hsa-miR-4792, hsa-miR-5787, hsa-miR-6798-5p, hsa-miR-6781-5p, hsa-miR-4419b, hsa-miR-4446-3p, hsa-miR-4259, hsa-miR-5572, hsa-miR-6075, hsa-miR-296-3p, hsa-miR-6891-5p, hsa-miR-4745-5p, hsa-miR-6775-5p, hsa-miR-6870-5p, hsa-miR-920, hsa-miR-4530, hsa-miR-6819-5p, hsa-miR-6825-5p, hsa-miR-7847-3p, hsa-miR-6131, hsa-miR-4433-3p, hsa-miR-1228-5p, hsa-miR-6743-5p, hsa-miR-1268a, hsa-miR-3917, hsa-miR-6786-5p, hsa-miR-3154, hsa-miR-638, hsa-miR-6741-5p, hsa-miR-6889-5p, hsa-miR-6840-3p, hsa-miR-6510-5p, hsa-miR-3188, hsa-miR-551b-5p, hsa-miR-5001-5p, hsa-miR-1268b, hsa-miR-7107-5p, hsa-miR-6824-5p, hsa-miR-6732-5p, hsa-miR-371a-5p, hsa-miR-6794-5p, hsa-miR-6779-5p, hsa-miR-4271, hsa-miR-5195-3p, hsa-miR-6762-5p, hsa-miR-939-5p, hsa-miR-1247-3p, hsa-miR-6777-5p, hsa-miR-6722-3p, hsa-miR-3656, hsa-miR-4688, hsa-miR-3195, hsa-miR-6766-5p, hsa-miR-4447, hsa-miR-4656, hsa-miR-7108-5p, hsa-miR-3191-3p, hsa-miR-1273g-3p, hsa-miR-4463, hsa-miR-2861, hsa-miR-3196, hsa-miR-6877-5p, hsa-miR-3679-5p, hsa-miR-4442, hsa-miR-6789-5p, hsa-miR-6782-5p, hsa-miR-486-3p, hsa-miR-6085, hsa-miR-4746-3p, hsa-miR-619-5p, hsa-miR-937-5p, hsa-miR-6803-5p, hsa-miR-4298, hsa-miR-4454, hsa-miR-4459, hsa-miR-7150, hsa-miR-6880-5p, hsa-miR-4449, hsa-miR-8063, hsa-miR-4695-5p, hsa-miR-6132, hsa-miR-6829-5p, hsa-miR-4486, hsa-miR-6805-3p, hsa-miR-6826-5p, hsa-miR-4508, hsa-miR-1343-5p, hsa-miR-7114-5p, hsa-miR-3622a-5p, hsa-miR-6765-5p, hsa-miR-7845-5p, hsa-miR-3960, hsa-miR-6749-5p, hsa-miR-1260b, hsa-miR-6799-5p, hsa-miR-4723-5p, hsa-miR-6784-5p, hsa-miR-5100, hsa-miR-6769b-5p, hsa-miR-1207-5p, hsa-miR-642a-3p, hsa-miR-4505, hsa-miR-4270, hsa-miR-6721-5p, hsa-miR-7111-5p, hsa-miR-6791-5p, hsa-miR-7109-5p, hsa-miR-4258, hsa-miR-6515-3p, hsa-miR-6851-5p, hsa-miR-6125, hsa-miR-4749-5p, hsa-miR-4726-5p, hsa-miR-4513, hsa-miR-6089, hsa-miR-6816-5p, hsa-miR-4466, hsa-miR-4488, hsa-miR-6752-5p, hsa-miR-4739, hsa-miR-760, hsa-miR-602, hsa-miR-423-5p, hsa-miR-92a-2-5p, hsa-miR-16-5p, hsa-miR-451a, hsa-miR-135a-3p, hsa-miR-486-5p, hsa-miR-4257, hsa-miR-92b-5p, hsa-miR-1915-3p, hsa-miR-718, hsa-miR-940, hsa-miR-296-5p, hsa-miR-23b-3p, hsa-miR-92a-3p, hsa-miR-658, hsa-miR-6842-5p, hsa-miR-6124, hsa-miR-6765-3p, hsa-miR-7106-5p, hsa-miR-4534, hsa-miR-92b-3p, hsa-miR-3135b, hsa-miR-4687-3p, hsa-miR-762, hsa-miR-3619-3p, hsa-miR-4467, hsa-miR-557, hsa-miR-1237-5p, hsa-miR-1908-5p, hsa-miR-4286, hsa-miR-6885-5p and hsa-miR-6763-5p, respectively), a congener thereof, a transcript thereof, or/and a variant or a derivative thereof. In this context, the gene, the congener, the transcript, the variant, and the derivative are as defined above.

The target nucleic acid is preferably a human gene comprising a nucleotide sequence represented by any of SEQ ID NOs: 1 to 871 or a transcript thereof, more preferably the transcript, i.e., a miRNA or its precursor RNA (pri-miRNA or pre-miRNA).

The first target gene is the hsa-miR-4783-3p gene, a congener thereof, a transcript thereof, or a variant or a derivative thereof. None of the previously known reports show that change in the expression of the gene or the transcript thereof can serve as a marker for breast cancer.

The second target gene is the hsa-miR-4730 gene, a congener thereof, a transcript thereof, or a variant or a derivative thereof. None of the previously known reports show that change in the expression of the gene or the transcript thereof can serve as a marker for breast cancer.

The third target gene is the hsa-miR-1307-3p gene, a congener thereof, a transcript thereof, or a variant or a derivative thereof. None of the previously known reports show that change in the expression of the gene or the transcript thereof can serve as a marker for breast cancer.

The fourth target gene is the hsa-miR-4634 gene, a congener thereof, a transcript thereof, or a variant or a derivative thereof. None of the previously known reports show that change in the expression of the gene or the transcript thereof can serve as a marker for breast cancer.

The fifth target gene is the hsa-miR-663a gene, a congener thereof, a transcript thereof, or a variant or a derivative thereof. None of the previously known reports show that change in the expression of the gene or the transcript thereof can serve as a marker for breast cancer.

The sixth target gene is the hsa-miR-4532 gene, a congener thereof, a transcript thereof, or a variant or a derivative thereof. None of the previously known reports show that change in the expression of the gene or the transcript thereof can serve as a marker for breast cancer.

The seventh target gene is the hsa-miR-7704 gene, a congener thereof, a transcript thereof, or a variant or a derivative thereof. None of the previously known reports show that change in the expression of the gene or the transcript thereof can serve as a marker for breast cancer.

The eighth target gene is the hsa-miR-3178 gene, a congener thereof, a transcript thereof, or a variant or a derivative thereof. None of the previously known reports show that change in the expression of the gene or the transcript thereof can serve as a marker for breast cancer.

The ninth target gene is the hsa-miR-6729-5p gene, a congener thereof, a transcript thereof, or a variant or a derivative thereof. None of the previously known reports show that change in the expression of the gene or the transcript thereof can serve as a marker for breast cancer.

The 10th target gene is the hsa-miR-6090 gene, a congener thereof, a transcript thereof, or a variant or a derivative thereof. None of the previously known reports show that change in the expression of the gene or the transcript thereof can serve as a marker for breast cancer.

The 11th target gene is the hsa-miR-4732-5p gene, a congener thereof, a transcript thereof, or a variant or a derivative thereof. None of the previously known reports show that change in the expression of the gene or the transcript thereof can serve as a marker for breast cancer.

The 12th target gene is the hsa-miR-3184-5p gene, a congener thereof, a transcript thereof, or a variant or a derivative thereof. None of the previously known reports show that change in the expression of the gene or the transcript thereof can serve as a marker for breast cancer.

The 13th target gene is the hsa-miR-6727-5p gene, a congener thereof, a transcript thereof, or a variant or a derivative thereof. None of the previously known reports show that change in the expression of the gene or the transcript thereof can serve as a marker for breast cancer.

The 14th target gene is the hsa-miR-6088 gene, a congener thereof, a transcript thereof, or a variant or a derivative thereof. None of the previously known reports show that change in the expression of the gene or the transcript thereof can serve as a marker for breast cancer.

The 15th target gene is the hsa-miR-4674 gene, a congener thereof, a transcript thereof, or a variant or a derivative thereof. None of the previously known reports show that change in the expression of the gene or the transcript thereof can serve as a marker for breast cancer.

The 16th target gene is the hsa-miR-8073 gene, a congener thereof, a transcript thereof, or a variant or a derivative thereof. None of the previously known reports show that change in the expression of the gene or the transcript thereof can serve as a marker for breast cancer.

The 17th target gene is the hsa-miR-4787-5p gene, a congener thereof, a transcript thereof, or a variant or a derivative thereof. None of the previously known reports show that change in the expression of the gene or the transcript thereof can serve as a marker for breast cancer.

The 18th target gene is the hsa-miR-1469 gene, a congener thereof, a transcript thereof, or a variant or a derivative thereof. None of the previously known reports show that change in the expression of the gene or the transcript thereof can serve as a marker for breast cancer.

The 19th target gene is the hsa-miR-125a-3p gene, a congener thereof, a transcript thereof, or a variant or a derivative thereof. None of the previously known reports show that change in the expression of the gene or the transcript thereof can serve as a marker for breast cancer.

The 20th target gene is the hsa-miR-1233-5p gene, a congener thereof, a transcript thereof, or a variant or a derivative thereof. None of the previously known reports show that change in the expression of the gene or the transcript thereof can serve as a marker for breast cancer.

The 21st target gene is the hsa-miR-885-3p gene, a congener thereof, a transcript thereof, or a variant or a derivative thereof. None of the previously known reports show that change in the expression of the gene or the transcript thereof can serve as a marker for breast cancer.

The 22nd target gene is the hsa-miR-6802-5p gene, a congener thereof, a transcript thereof, or a variant or a derivative thereof. None of the previously known reports show that change in the expression of the gene or the transcript thereof can serve as a marker for breast cancer.

The 23rd target gene is the hsa-miR-328-5p gene, a congener thereof, a transcript thereof, or a variant or a derivative thereof. None of the previously known reports show that change in the expression of the gene or the transcript thereof can serve as a marker for breast cancer.

The 24th target gene is the hsa-miR-6787-5p gene, a congener thereof, a transcript thereof, or a variant or a derivative thereof. None of the previously known reports show that change in the expression of the gene or the transcript thereof can serve as a marker for breast cancer.

The 25th target gene is the hsa-miR-8069 gene, a congener thereof, a transcript thereof, or a variant or a derivative thereof. None of the previously known reports show that change in the expression of the gene or the transcript thereof can serve as a marker for breast cancer.

The 26th target gene is the hsa-miR-6875-5p gene, a congener thereof, a transcript thereof, or a variant or a derivative thereof. None of the previously known reports show that change in the expression of the gene or the transcript thereof can serve as a marker for breast cancer.

The 27th target gene is the hsa-miR-1246 gene, a congener thereof, a transcript thereof, or a variant or a derivative thereof. None of the previously known reports show that change in the expression of the gene or the transcript thereof can serve as a marker for breast cancer.

The 28th target gene is the hsa-miR-4734 gene, a congener thereof, a transcript thereof, or a variant or a derivative thereof. None of the previously known reports show that change in the expression of the gene or the transcript thereof can serve as a marker for breast cancer.

The 29th target gene is the hsa-miR-6757-5p gene, a congener thereof, a transcript thereof, or a variant or a derivative thereof. None of the previously known reports show that change in the expression of the gene or the transcript thereof can serve as a marker

The 30th target gene is the hsa-miR-6756-5p gene, a congener thereof, a transcript thereof, or a variant or a derivative thereof. None of the previously known reports show that change in the expression of the gene or the transcript thereof can serve as a marker for breast cancer.

The 31st target gene is the hsa-miR-3665 gene, a congener thereof, a transcript thereof, or a variant or a derivative thereof. None of the previously known reports show that change in the expression of the gene or the transcript thereof can serve as a marker for breast cancer.

The 32nd target gene is the hsa-miR-6836-3p gene, a congener thereof, a transcript thereof, or a variant or a derivative thereof. None of the previously known reports show that change in the expression of the gene or the transcript thereof can serve as a marker for breast cancer.

The 33rd target gene is the hsa-miR-6821-5p gene, a congener thereof, a transcript thereof, or a variant or a derivative thereof. None of the previously known reports show that change in the expression of the gene or the transcript thereof can serve as a marker for breast cancer.

The 34th target gene is the hsa-miR-6805-5p gene, a congener thereof, a transcript thereof, or a variant or a derivative thereof. None of the previously known reports show that change in the expression of the gene or the transcript thereof can serve as a marker for breast cancer.

The 35th target gene is the hsa-miR-4728-5p gene, a congener thereof, a transcript thereof, or a variant or a derivative thereof. None of the previously known reports show that change in the expression of the gene or the transcript thereof can serve as a marker for breast cancer.

The 36th target gene is the hsa-miR-6726-5p gene, a congener thereof, a transcript thereof, or a variant or a derivative thereof. None of the previously known reports show that change in the expression of the gene or the transcript thereof can serve as a marker for breast cancer.

The 37th target gene is the hsa-miR-197-5p gene, a congener thereof, a transcript thereof, or a variant or a derivative thereof. None of the previously known reports show that change in the expression of the gene or the transcript thereof can serve as a marker for breast cancer.

The 38th target gene is the hsa-miR-149-3p gene, a congener thereof, a transcript thereof, or a variant or a derivative thereof. None of the previously known reports show that change in the expression of the gene or the transcript thereof can serve as a marker for breast cancer.

The 39th target gene is the hsa-miR-6850-5p gene, a congener thereof, a transcript thereof, or a variant or a derivative thereof. None of the previously known reports show that change in the expression of the gene or the transcript thereof can serve as a marker for breast cancer.

The 40th target gene is the hsa-miR-4476 gene, a congener thereof, a transcript thereof, or a variant or a derivative thereof. None of the previously known reports show that change in the expression of the gene or the transcript thereof can serve as a marker for breast cancer.

The 41st target gene is the hsa-miR-6858-5p gene, a congener thereof, a transcript thereof, or a variant or a derivative thereof. None of the previously known reports show that change in the expression of the gene or the transcript thereof can serve as a marker for breast cancer.

The 42nd target gene is the hsa-miR-564 gene, a congener thereof, a transcript thereof, or a variant or a derivative thereof. None of the previously known reports show that change in the expression of the gene or the transcript thereof can serve as a marker for breast cancer.

The 43rd target gene is the hsa-miR-4763-3p gene, a congener thereof, a transcript thereof, or a variant or a derivative thereof. None of the previously known reports show that change in the expression of the gene or the transcript thereof can serve as a marker for breast cancer.

The 44th target gene is the hsa-miR-575 gene, a congener thereof, a transcript thereof, or a variant or a derivative thereof. None of the previously known reports show that change in the expression of the gene or the transcript thereof can serve as a marker for breast cancer.

The 45th target gene is the hsa-miR-6771-5p gene, a congener thereof, a transcript thereof, or a variant or a derivative thereof. None of the previously known reports show that change in the expression of the gene or the transcript thereof can serve as a marker for breast cancer.

The 46th target gene is the hsa-miR-1231 gene, a congener thereof, a transcript thereof, or a variant or a derivative thereof. None of the previously known reports show that change in the expression of the gene or the transcript thereof can serve as a marker for breast cancer.

The 47th target gene is the hsa-miR-1908-3p gene, a congener thereof, a transcript thereof, or a variant or a derivative thereof. None of the previously known reports show that change in the expression of the gene or the transcript thereof can serve as a marker for breast cancer.

The 48th target gene is the hsa-miR-150-3p gene, a congener thereof, a transcript thereof, or a variant or a derivative thereof. None of the previously known reports show that change in the expression of the gene or the transcript thereof can serve as a marker for breast cancer.

The 49th target gene is the hsa-miR-3937 gene, a congener thereof, a transcript thereof, or a variant or a derivative thereof. None of the previously known reports show that change in the expression of the gene or the transcript thereof can serve as a marker for breast cancer.

The 50th target gene is the hsa-miR-887-3p gene, a congener thereof, a transcript thereof, or a variant or a derivative thereof. None of the previously known reports show that change in the expression of the gene or the transcript thereof can serve as a marker for breast cancer.

The 51st target gene is the hsa-miR-3940-5p gene, a congener thereof, a transcript thereof, or a variant or a derivative thereof. None of the previously known reports show that change in the expression of the gene or the transcript thereof can serve as a marker for breast cancer.

The 52nd target gene is the hsa-miR-4741 gene, a congener thereof, a transcript thereof, or a variant or a derivative thereof. None of the previously known reports show that change in the expression of the gene or the transcript thereof can serve as a marker for breast cancer.

The 53rd target gene is the hsa-miR-6808-5p gene, a congener thereof, a transcript thereof, or a variant or a derivative thereof. None of the previously known reports show that change in the expression of the gene or the transcript thereof can serve as a marker for breast cancer.

The 54th target gene is the hsa-miR-6869-5p gene, a congener thereof, a transcript thereof, or a variant or a derivative thereof. None of the previously known reports show that change in the expression of the gene or the transcript thereof can serve as a marker for breast cancer.

The 55th target gene is the hsa-miR-5090 gene, a congener thereof, a transcript thereof, or a variant or a derivative thereof. None of the previously known reports show that change in the expression of the gene or the transcript thereof can serve as a marker for breast cancer.

The 56th target gene is the hsa-miR-615-5p gene, a congener thereof, a transcript thereof, or a variant or a derivative thereof. None of the previously known reports show that change in the expression of the gene or the transcript thereof can serve as a marker for breast cancer.

The 57th target gene is the hsa-miR-8072 gene, a congener thereof, a transcript thereof, or a variant or a derivative thereof. None of the previously known reports show that change in the expression of the gene or the transcript thereof can serve as a marker for breast cancer.

The 58th target gene is the hsa-miR-128-1-5p gene, a congener thereof, a transcript thereof, or a variant or a derivative thereof. None of the previously known reports show that change in the expression of the gene or the transcript thereof can serve as a marker for breast cancer.

The 59th target gene is the hsa-miR-1238-5p gene, a congener thereof, a transcript thereof, or a variant or a derivative thereof. None of the previously known reports show that change in the expression of the gene or the transcript thereof can serve as a marker for breast cancer.

The 60th target gene is the hsa-miR-365a-5p gene, a congener thereof, a transcript thereof, or a variant or a derivative thereof. None of the previously known reports show that change in the expression of the gene or the transcript thereof can serve as a marker for breast cancer.

The 61st target gene is the hsa-miR-204-3p gene, a congener thereof, a transcript thereof, or a variant or a derivative thereof. None of the previously known reports show that change in the expression of the gene or the transcript thereof can serve as a marker for breast cancer.

The 62nd target gene is the hsa-miR-4492 gene, a congener thereof, a transcript thereof, or a variant or a derivative thereof. None of the previously known reports show that change in the expression of the gene or the transcript thereof can serve as a marker for breast cancer.

The 63rd target gene is the hsa-miR-6785-5p gene, a congener thereof, a transcript thereof, or a variant or a derivative thereof. None of the previously known reports show that change in the expression of the gene or the transcript thereof can serve as a marker for breast cancer.

The 64th target gene is the hsa-miR-6511a-5p gene, a congener thereof, a transcript thereof, or a variant or a derivative thereof. None of the previously known reports show that change in the expression of the gene or the transcript thereof can serve as a marker for breast cancer.

The 65th target gene is the hsa-miR-4525 gene, a congener thereof, a transcript thereof, or a variant or a derivative thereof. None of the previously known reports show that change in the expression of the gene or the transcript thereof can serve as a marker for breast cancer.

The 66th target gene is the hsa-miR-1915-5p gene, a congener thereof, a transcript thereof, or a variant or a derivative thereof. None of the previously known reports show that change in the expression of the gene or the transcript thereof can serve as a marker for breast cancer.

The 67th target gene is the hsa-miR-3180 gene, a congener thereof, a transcript thereof, or a variant or a derivative thereof. None of the previously known reports show that change in the expression of the gene or the transcript thereof can serve as a marker for breast cancer.

The 68th target gene is the hsa-miR-6879-5p gene, a congener thereof, a transcript thereof, or a variant or a derivative thereof. None of the previously known reports show that change in the expression of the gene or the transcript thereof can serve as a marker for breast cancer.

The 69th target gene is the hsa-miR-1199-5p gene, a congener thereof, a transcript thereof, or a variant or a derivative thereof. None of the previously known reports show that change in the expression of the gene or the transcript thereof can serve as a marker for breast cancer.

The 70th target gene is the hsa-miR-6746-5p gene, a congener thereof, a transcript thereof, or a variant or a derivative thereof. None of the previously known reports show that change in the expression of the gene or the transcript thereof can serve as a marker for breast cancer.

The 71st target gene is the hsa-miR-711 gene, a congener thereof, a transcript thereof, or a variant or a derivative thereof. None of the previously known reports show that change in the expression of the gene or the transcript thereof can serve as a marker for breast cancer.

The 72nd target gene is the hsa-miR-663b gene, a congener thereof, a transcript thereof, or a variant or a derivative thereof. None of the previously known reports show that change in the expression of the gene or the transcript thereof can serve as a marker for breast cancer.

The 73rd target gene is the hsa-miR-4707-3p gene, a congener thereof, a transcript thereof, or a variant or a derivative thereof. None of the previously known reports show that change in the expression of the gene or the transcript thereof can serve as a marker for breast cancer.

The 74th target gene is the hsa-miR-6893-5p gene, a congener thereof, a transcript thereof, or a variant or a derivative thereof. None of the previously known reports show that change in the expression of the gene or the transcript thereof can serve as a marker for breast cancer.

The 75th target gene is the hsa-miR-4675 gene, a congener thereof, a transcript thereof, or a variant or a derivative thereof. None of the previously known reports show that change in the expression of the gene or the transcript thereof can serve as a marker for breast cancer.

The 76th target gene is the hsa-miR-4638-5p gene, a congener thereof, a transcript thereof, or a variant or a derivative thereof. None of the previously known reports show that change in the expression of the gene or the transcript thereof can serve as a marker for breast cancer.

The 77th target gene is the hsa-miR-4651 gene, a congener thereof, a transcript thereof, or a variant or a derivative thereof. None of the previously known reports show that change in the expression of the gene or the transcript thereof can serve as a marker for breast cancer.

The 78th target gene is the hsa-miR-6087 gene, a congener thereof, a transcript thereof, or a variant or a derivative thereof. None of the previously known reports show that change in the expression of the gene or the transcript thereof can serve as a marker for breast cancer.

The 79th target gene is the hsa-miR-4665-5p gene, a congener thereof, a transcript thereof, or a variant or a derivative thereof. None of the previously known reports show that change in the expression of the gene or the transcript thereof can serve as a marker for breast cancer.

The 80th target gene is the hsa-miR-4758-5p gene, a congener thereof, a transcript thereof, or a variant or a derivative thereof. None of the previously known reports show that change in the expression of the gene or the transcript thereof can serve as a marker for breast cancer.

The 81st target gene is the hsa-miR-6887-5p gene, a congener thereof, a transcript thereof, or a variant or a derivative thereof. None of the previously known reports show that change in the expression of the gene or the transcript thereof can serve as a marker for breast cancer.

The 82nd target gene is the hsa-miR-3620-5p gene, a congener thereof, a transcript thereof, or a variant or a derivative thereof. None of the previously known reports show that change in the expression of the gene or the transcript thereof can serve as a marker for breast cancer.

The 83rd target gene is the hsa-miR-1909-3p gene, a congener thereof, a transcript thereof, or a variant or a derivative thereof. None of the previously known reports show that change in the expression of the gene or the transcript thereof can serve as a marker for breast cancer.

The 84th target gene is the hsa-miR-7641 gene, a congener thereof, a transcript thereof, or a variant or a derivative thereof. None of the previously known reports show that change in the expression of the gene or the transcript thereof can serve as a marker for breast cancer.

The 85th target gene is the hsa-miR-6724-5p gene, a congener thereof, a transcript thereof, or a variant or a derivative thereof. None of the previously known reports show that change in the expression of the gene or the transcript thereof can serve as a marker for breast cancer.

The 86th target gene is the hsa-miR-1343-3p gene, a congener thereof, a transcript thereof, or a variant or a derivative thereof. None of the previously known reports show that change in the expression of the gene or the transcript thereof can serve as a marker for breast cancer.

The 87th target gene is the hsa-miR-6780b-5p gene, a congener thereof, a transcript thereof, or a variant or a derivative thereof. None of the previously known reports show that change in the expression of the gene or the transcript thereof can serve as a marker for breast cancer.

The 88th target gene is the hsa-miR-4484 gene, a congener thereof, a transcript thereof, or a variant or a derivative thereof. None of the previously known reports show that change in the expression of the gene or the transcript thereof can serve as a marker for breast cancer.

The 89th target gene is the hsa-miR-4690-5p gene, a congener thereof, a transcript thereof, or a variant or a derivative thereof. None of the previously known reports show that change in the expression of the gene or the transcript thereof can serve as a marker for breast cancer.

The 90th target gene is the hsa-miR-4429 gene, a congener thereof, a transcript thereof, or a variant or a derivative thereof. None of the previously known reports show that change in the expression of the gene or the transcript thereof can serve as a marker for breast cancer.

The 91st target gene is the hsa-miR-1227-5p gene, a congener thereof, a transcript thereof, or a variant or a derivative thereof. None of the previously known reports show that change in the expression of the gene or the transcript thereof can serve as a marker for breast cancer.

The 92nd target gene is the hsa-miR-4725-3p gene, a congener thereof, a transcript thereof, or a variant or a derivative thereof. None of the previously known reports show that change in the expression of the gene or the transcript thereof can serve as a marker for breast cancer.

The 93rd target gene is the hsa-miR-6861-5p gene, a congener thereof, a transcript thereof, or a variant or a derivative thereof. None of the previously known reports show that change in the expression of the gene or the transcript thereof can serve as a marker for breast cancer.

The 94th target gene is the hsa-miR-6812-5p gene, a congener thereof, a transcript thereof, or a variant or a derivative thereof. None of the previously known reports show that change in the expression of the gene or the transcript thereof can serve as a marker for breast cancer.

The 95th target gene is the hsa-miR-3197 gene, a congener thereof, a transcript thereof, or a variant or a derivative thereof. None of the previously known reports show that change in the expression of the gene or the transcript thereof can serve as a marker for breast cancer.

The 96th target gene is the hsa-miR-8059 gene, a congener thereof, a transcript thereof, or a variant or a derivative thereof. None of the previously known reports show that change in the expression of the gene or the transcript thereof can serve as a marker for breast cancer.

The 97th target gene is the hsa-miR-3185 gene, a congener thereof, a transcript thereof, or a variant or a derivative thereof. None of the previously known reports show that change in the expression of the gene or the transcript thereof can serve as a marker for breast cancer.

The 98th target gene is the hsa-miR-4706 gene, a congener thereof, a transcript thereof, or a variant or a derivative thereof. None of the previously known reports show that change in the expression of the gene or the transcript thereof can serve as a marker for breast cancer.

The 99th target gene is the hsa-miR-4497 gene, a congener thereof, a transcript thereof, or a variant or a derivative thereof. None of the previously known reports show that change in the expression of the gene or the transcript thereof can serve as a marker for breast cancer.

The 100th target gene is the hsa-miR-3131 gene, a congener thereof, a transcript thereof, or a variant or a derivative thereof. None of the previously known reports show that change in the expression of the gene or the transcript thereof can serve as a marker for breast cancer.

The 101st target gene is the hsa-miR-6806-5p gene, a congener thereof, a transcript thereof, or a variant or a derivative thereof. None of the previously known reports show that change in the expression of the gene or the transcript thereof can serve as a marker for breast cancer.

The 102nd target gene is the hsa-miR-187-5p gene, a congener thereof, a transcript thereof, or a variant or a derivative thereof. None of the previously known reports show that change in the expression of the gene or the transcript thereof can serve as a marker for breast cancer.

The 103rd target gene is the hsa-miR-3180-3p gene, a congener thereof, a transcript thereof, or a variant or a derivative thereof. None of the previously known reports show that change in the expression of the gene or the transcript thereof can serve as a marker for breast cancer.

The 104th target gene is the hsa-miR-6848-5p gene, a congener thereof, a transcript thereof, or a variant or a derivative thereof. None of the previously known reports show that change in the expression of the gene or the transcript thereof can serve as a marker for breast cancer.

The 105th target gene is the hsa-miR-6820-5p gene, a congener thereof, a transcript thereof, or a variant or a derivative thereof. None of the previously known reports show that change in the expression of the gene or the transcript thereof can serve as a marker for breast cancer.

The 106th target gene is the hsa-miR-6800-5p gene, a congener thereof, a transcript thereof, or a variant or a derivative thereof. None of the previously known reports show that change in the expression of the gene or the transcript thereof can serve as a marker for breast cancer.

The 107th target gene is the hsa-miR-6717-5p gene, a congener thereof, a transcript thereof, or a variant or a derivative thereof. None of the previously known reports show that change in the expression of the gene or the transcript thereof can serve as a marker for breast cancer.

The 108th target gene is the hsa-miR-6795-5p gene, a congener thereof, a transcript thereof, or a variant or a derivative thereof. None of the previously known reports show that change in the expression of the gene or the transcript thereof can serve as a marker for breast cancer.

The 109th target gene is the hsa-miR-4632-5p gene, a congener thereof, a transcript thereof, or a variant or a derivative thereof. None of the previously known reports show that change in the expression of the gene or the transcript thereof can serve as a marker for breast cancer.

The 110th target gene is the hsa-miR-665 gene, a congener thereof, a transcript thereof, or a variant or a derivative thereof. None of the previously known reports show that change in the expression of the gene or the transcript thereof can serve as a marker for breast cancer.

The 111th target gene is the hsa-miR-6778-5p gene, a congener thereof, a transcript thereof, or a variant or a derivative thereof. None of the previously known reports show that change in the expression of the gene or the transcript thereof can serve as a marker for breast cancer.

The 112th target gene is the hsa-miR-3663-3p gene, a congener thereof, a transcript thereof, or a variant or a derivative thereof. None of the previously known reports show that change in the expression of the gene or the transcript thereof can serve as a marker for breast cancer.

The 113th target gene is the hsa-miR-4689 gene, a congener thereof, a transcript thereof, or a variant or a derivative thereof. None of the previously known reports show that change in the expression of the gene or the transcript thereof can serve as a marker for breast cancer.

The 114th target gene is the hsa-miR-211-3p gene, a congener thereof, a transcript thereof, or a variant or a derivative thereof. None of the previously known reports show that change in the expression of the gene or the transcript thereof can serve as a marker for breast cancer.

The 115th target gene is the hsa-miR-6511b-5p gene, a congener thereof, a transcript thereof, or a variant or a derivative thereof. None of the previously known reports show that change in the expression of the gene or the transcript thereof can serve as a marker for breast cancer.

The 116th target gene is the hsa-miR-4750-5p gene, a congener thereof, a transcript thereof, or a variant or a derivative thereof. None of the previously known reports show that change in the expression of the gene or the transcript thereof can serve as a marker for breast cancer.

The 117th target gene is the hsa-miR-6126 gene, a congener thereof, a transcript thereof, or a variant or a derivative thereof. None of the previously known reports show that change in the expression of the gene or the transcript thereof can serve as a marker for breast cancer.

The 118th target gene is the hsa-miR-614 gene, a congener thereof, a transcript thereof, or a variant or a derivative thereof. None of the previously known reports show that change in the expression of the gene or the transcript thereof can serve as a marker for breast cancer.

The 119th target gene is the hsa-miR-7110-5p gene, a congener thereof, a transcript thereof, or a variant or a derivative thereof. None of the previously known reports show that change in the expression of the gene or the transcript thereof can serve as a marker for breast cancer.

The 120th target gene is the hsa-miR-744-5p gene, a congener thereof, a transcript thereof, or a variant or a derivative thereof. None of the previously known reports show that change in the expression of the gene or the transcript thereof can serve as a marker for breast cancer.

The 121st target gene is the hsa-miR-6769a-5p gene, a congener thereof, a transcript thereof, or a variant or a derivative thereof. None of the previously known reports show that change in the expression of the gene or the transcript thereof can serve as a marker for breast cancer.

The 122nd target gene is the hsa-miR-4792 gene, a congener thereof, a transcript thereof, or a variant or a derivative thereof. None of the previously known reports show that change in the expression of the gene or the transcript thereof can serve as a marker for breast cancer.

The 123rd target gene is the hsa-miR-5787 gene, a congener thereof, a transcript thereof, or a variant or a derivative thereof. None of the previously known reports show that change in the expression of the gene or the transcript thereof can serve as a marker for breast cancer.

The 124th target gene is the hsa-miR-6798-5p gene, a congener thereof, a transcript thereof, or a variant or a derivative thereof. None of the previously known reports show that change in the expression of the gene or the transcript thereof can serve as a marker for breast cancer.

The 125th target gene is the hsa-miR-6781-5p gene, a congener thereof, a transcript thereof, or a variant or a derivative thereof. None of the previously known reports show that change in the expression of the gene or the transcript thereof can serve as a marker for breast cancer.

The 126th target gene is the hsa-miR-4419b gene, a congener thereof, a transcript thereof, or a variant or a derivative thereof. None of the previously known reports show that change in the expression of the gene or the transcript thereof can serve as a marker for breast cancer.

The 127th target gene is the hsa-miR-4446-3p gene, a congener thereof, a transcript thereof, or a variant or a derivative thereof. None of the previously known reports show that change in the expression of the gene or the transcript thereof can serve as a marker for breast cancer.

The 128th target gene is the hsa-miR-4259 gene, a congener thereof, a transcript thereof, or a variant or a derivative thereof. None of the previously known reports show that change in the expression of the gene or the transcript thereof can serve as a marker for breast cancer.

The 129th target gene is the hsa-miR-5572 gene, a congener thereof, a transcript thereof, or a variant or a derivative thereof. None of the previously known reports show that change in the expression of the gene or the transcript thereof can serve as a marker for breast cancer.

The 130th target gene is the hsa-miR-6075 gene, a congener thereof, a transcript thereof, or a variant or a derivative thereof. None of the previously known reports show that change in the expression of the gene or the transcript thereof can serve as a marker for breast cancer.

The 131st target gene is the hsa-miR-296-3p gene, a congener thereof, a transcript thereof, or a variant or a derivative thereof. None of the previously known reports show that change in the expression of the gene or the transcript thereof can serve as a marker for breast cancer.

The 132nd target gene is the hsa-miR-6891-5p gene, a congener thereof, a transcript thereof, or a variant or a derivative thereof. None of the previously known reports show that change in the expression of the gene or the transcript thereof can serve as a marker for breast cancer.

The 133rd target gene is the hsa-miR-4745-5p gene, a congener thereof, a transcript thereof, or a variant or a derivative thereof. None of the previously known reports show that change in the expression of the gene or the transcript thereof can serve as a marker for breast cancer.

The 134th target gene is the hsa-miR-6775-5p gene, a congener thereof, a transcript thereof, or a variant or a derivative thereof. None of the previously known reports show that change in the expression of the gene or the transcript thereof can serve as a marker for breast cancer.

The 135th target gene is the hsa-miR-6870-5p gene, a congener thereof, a transcript thereof, or a variant or a derivative thereof. None of the previously known reports show that change in the expression of the gene or the transcript thereof can serve as a marker for breast cancer.

The 136th target gene is the hsa-miR-920 gene, a congener thereof, a transcript thereof, or a variant or a derivative thereof. None of the previously known reports show that change in the expression of the gene or the transcript thereof can serve as a marker for breast cancer.

The 137th target gene is the hsa-miR-4530 gene, a congener thereof, a transcript thereof, or a variant or a derivative thereof. None of the previously known reports show that change in the expression of the gene or the transcript thereof can serve as a marker for breast cancer.

The 138th target gene is the hsa-miR-6819-5p gene, a congener thereof, a transcript thereof, or a variant or a derivative thereof. None of the previously known reports show that change in the expression of the gene or the transcript thereof can serve as a marker for breast cancer.

The 139th target gene is the hsa-miR-6825-5p gene, a congener thereof, a transcript thereof, or a variant or a derivative thereof. None of the previously known reports show that change in the expression of the gene or the transcript thereof can serve as a marker for breast cancer.

The 140th target gene is the hsa-miR-7847-3p gene, a congener thereof, a transcript thereof, or a variant or a derivative thereof. None of the previously known reports show that change in the expression of the gene or the transcript thereof can serve as a marker for breast cancer.

The 141st target gene is the hsa-miR-6131 gene, a congener thereof, a transcript thereof, or a variant or a derivative thereof. None of the previously known reports show that change in the expression of the gene or the transcript thereof can serve as a marker for breast cancer.

The 142nd target gene is the hsa-miR-4433-3p gene, a congener thereof, a transcript thereof, or a variant or a derivative thereof. None of the previously known reports show that change in the expression of the gene or the transcript thereof can serve as a marker for breast cancer.

The 143rd target gene is the hsa-miR-1228-5p gene, a congener thereof, a transcript thereof, or a variant or a derivative thereof. None of the previously known reports show that change in the expression of the gene or the transcript thereof can serve as a marker for breast cancer.

The 144th target gene is the hsa-miR-6743-5p gene, a congener thereof, a transcript thereof, or a variant or a derivative thereof. None of the previously known reports show that change in the expression of the gene or the transcript thereof can serve as a marker for breast cancer.

The 145th target gene is the hsa-miR-1268a gene, a congener thereof, a transcript thereof, or a variant or a derivative thereof. None of the previously known reports show that change in the expression of the gene or the transcript thereof can serve as a marker for breast cancer.

The 146th target gene is the hsa-miR-3917 gene, a congener thereof, a transcript thereof, or a variant or a derivative thereof. None of the previously known reports show that change in the expression of the gene or the transcript thereof can serve as a marker for breast cancer.

The 147th target gene is the hsa-miR-6786-5p gene, a congener thereof, a transcript thereof, or a variant or a derivative thereof. None of the previously known reports show that change in the expression of the gene or the transcript thereof can serve as a marker for breast cancer.

The 148th target gene is the hsa-miR-3154 gene, a congener thereof, a transcript thereof, or a variant or a derivative thereof. None of the previously known reports show that change in the expression of the gene or the transcript thereof can serve as a marker for breast cancer.

The 149th target gene is the hsa-miR-638 gene, a congener thereof, a transcript thereof, or a variant or a derivative thereof. None of the previously known reports show that change in the expression of the gene or the transcript thereof can serve as a marker for breast cancer.

The 150th target gene is the hsa-miR-6741-5p gene, a congener thereof, a transcript thereof, or a variant or a derivative thereof. None of the previously known reports show that change in the expression of the gene or the transcript thereof can serve as a marker for breast cancer.

The 151st target gene is the hsa-miR-6889-5p gene, a congener thereof, a transcript thereof, or a variant or a derivative thereof. None of the previously known reports show that change in the expression of the gene or the transcript thereof can serve as a marker for breast cancer.

The 152nd target gene is the hsa-miR-6840-3p gene, a congener thereof, a transcript thereof, or a variant or a derivative thereof. None of the previously known reports show that change in the expression of the gene or the transcript thereof can serve as a marker for breast cancer.

The 153rd target gene is the hsa-miR-6510-5p gene, a congener thereof, a transcript thereof, or a variant or a derivative thereof. None of the previously known reports show that change in the expression of the gene or the transcript thereof can serve as a marker for breast cancer.

The 154th target gene is the hsa-miR-3188 gene, a congener thereof, a transcript thereof, or a variant or a derivative thereof. None of the previously known reports show that change in the expression of the gene or the transcript thereof can serve as a marker for breast cancer.

The 155th target gene is the hsa-miR-551b-5p gene, a congener thereof, a transcript thereof, or a variant or a derivative thereof. None of the previously known reports show that change in the expression of the gene or the transcript thereof can serve as a marker for breast cancer.

The 156th target gene is the hsa-miR-5001-5p gene, a congener thereof, a transcript thereof, or a variant or a derivative thereof. None of the previously known reports show that change in the expression of the gene or the transcript thereof can serve as a marker for breast cancer.

The 157th target gene is the hsa-miR-1268b gene, a congener thereof, a transcript thereof, or a variant or a derivative thereof. None of the previously known reports show that change in the expression of the gene or the transcript thereof can serve as a marker for breast cancer.

The 158th target gene is the hsa-miR-7107-5p gene, a congener thereof, a transcript thereof, or a variant or a derivative thereof. None of the previously known reports show that change in the expression of the gene or the transcript thereof can serve as a marker for breast cancer.

The 159th target gene is the hsa-miR-6824-5p gene, a congener thereof, a transcript thereof, or a variant or a derivative thereof. None of the previously known reports show that change in the expression of the gene or the transcript thereof can serve as a marker for breast cancer.

The 160th target gene is the hsa-miR-6732-5p gene, a congener thereof, a transcript thereof, or a variant or a derivative thereof. None of the previously known reports show that change in the expression of the gene or the transcript thereof can serve as a marker for breast cancer.

The 161st target gene is the hsa-miR-371a-5p gene, a congener thereof, a transcript thereof, or a variant or a derivative thereof. None of the previously known reports show that change in the expression of the gene or the transcript thereof can serve as a marker for breast cancer.

The 162nd target gene is the hsa-miR-6794-5p gene, a congener thereof, a transcript thereof, or a variant or a derivative thereof. None of the previously known reports show that change in the expression of the gene or the transcript thereof can serve as a marker for breast cancer.

The 163rd target gene is the hsa-miR-6779-5p gene, a congener thereof, a transcript thereof, or a variant or a derivative thereof. None of the previously known reports show that change in the expression of the gene or the transcript thereof can serve as a marker for breast cancer.

The 164th target gene is the hsa-miR-4271 gene, a congener thereof, a transcript thereof, or a variant or a derivative thereof. None of the previously known reports show that change in the expression of the gene or the transcript thereof can serve as a marker for breast cancer.

The 165th target gene is the hsa-miR-5195-3p gene, a congener thereof, a transcript thereof, or a variant or a derivative thereof. None of the previously known reports show that change in the expression of the gene or the transcript thereof can serve as a marker for breast cancer.

The 166th target gene is the hsa-miR-6762-5p gene, a congener thereof, a transcript thereof, or a variant or a derivative thereof. None of the previously known reports show that change in the expression of the gene or the transcript thereof can serve as a marker for breast cancer.

The 167th target gene is the hsa-miR-939-5p gene, a congener thereof, a transcript thereof, or a variant or a derivative thereof. None of the previously known reports show that change in the expression of the gene or the transcript thereof can serve as a marker for breast cancer.

The 168th target gene is the hsa-miR-1247-3p gene, a congener thereof, a transcript thereof, or a variant or a derivative thereof. None of the previously known reports show that change in the expression of the gene or the transcript thereof can serve as a marker for breast cancer.

The 169th target gene is the hsa-miR-6777-5p gene, a congener thereof, a transcript thereof, or a variant or a derivative thereof. None of the previously known reports show that change in the expression of the gene or the transcript thereof can serve as a marker for breast cancer.

The 170th target gene is the hsa-miR-6722-3p gene, a congener thereof, a transcript thereof, or a variant or a derivative thereof. None of the previously known reports show that change in the expression of the gene or the transcript thereof can serve as a marker for breast cancer.

The 171st target gene is the hsa-miR-3656 gene, a congener thereof, a transcript thereof, or a variant or a derivative thereof. None of the previously known reports show that change in the expression of the gene or the transcript thereof can serve as a marker for breast cancer.

The 172nd target gene is the hsa-miR-4688 gene, a congener thereof, a transcript thereof, or a variant or a derivative thereof. None of the previously known reports show that change in the expression of the gene or the transcript thereof can serve as a marker for breast cancer.

The 173rd target gene is the hsa-miR-3195 gene, a congener thereof, a transcript thereof, or a variant or a derivative thereof. None of the previously known reports show that change in the expression of the gene or the transcript thereof can serve as a marker for breast cancer.

The 174th target gene is the hsa-miR-6766-5p gene, a congener thereof, a transcript thereof, or a variant or a derivative thereof. None of the previously known reports show that change in the expression of the gene or the transcript thereof can serve as a marker for breast cancer.

The 175th target gene is the hsa-miR-4447 gene, a congener thereof, a transcript thereof, or a variant or a derivative thereof. None of the previously known reports show that change in the expression of the gene or the transcript thereof can serve as a marker for breast cancer.

The 176th target gene is the hsa-miR-4656 gene, a congener thereof, a transcript thereof, or a variant or a derivative thereof. None of the previously known reports show that change in the expression of the gene or the transcript thereof can serve as a marker for breast cancer.

The 177th target gene is the hsa-miR-7108-5p gene, a congener thereof, a transcript thereof, or a variant or a derivative thereof. None of the previously known reports show that change in the expression of the gene or the transcript thereof can serve as a marker for breast cancer.

The 178th target gene is the hsa-miR-3191-3p gene, a congener thereof, a transcript thereof, or a variant or a derivative thereof. None of the previously known reports show that change in the expression of the gene or the transcript thereof can serve as a marker for breast cancer.

The 179th target gene is the hsa-miR-1273g-3p gene, a congener thereof, a transcript thereof, or a variant or a derivative thereof. None of the previously known reports show that change in the expression of the gene or the transcript thereof can serve as a marker for breast cancer.

The 180th target gene is the hsa-miR-4463 gene, a congener thereof, a transcript thereof, or a variant or a derivative thereof. None of the previously known reports show that change in the expression of the gene or the transcript thereof can serve as a marker for breast cancer.

The 181st target gene is the hsa-miR-2861 gene, a congener thereof, a transcript thereof, or a variant or a derivative thereof. None of the previously known reports show that change in the expression of the gene or the transcript thereof can serve as a marker for breast cancer.

The 182nd target gene is the hsa-miR-3196 gene, a congener thereof, a transcript thereof, or a variant or a derivative thereof. None of the previously known reports show that change in the expression of the gene or the transcript thereof can serve as a marker for breast cancer.

The 183rd target gene is the hsa-miR-6877-5p gene, a congener thereof, a transcript thereof, or a variant or a derivative thereof. None of the previously known reports show that change in the expression of the gene or the transcript thereof can serve as a marker for breast cancer.

The 184th target gene is the hsa-miR-3679-5p gene, a congener thereof, a transcript thereof, or a variant or a derivative thereof. None of the previously known reports show that change in the expression of the gene or the transcript thereof can serve as a marker for breast cancer.

The 185th target gene is the hsa-miR-4442 gene, a congener thereof, a transcript thereof, or a variant or a derivative thereof. None of the previously known reports show that change in the expression of the gene or the transcript thereof can serve as a marker for breast cancer.

The 186th target gene is the hsa-miR-6789-5p gene, a congener thereof, a transcript thereof, or a variant or a derivative thereof. None of the previously known reports show that change in the expression of the gene or the transcript thereof can serve as a marker for breast cancer.

The 187th target gene is the hsa-miR-6782-5p gene, a congener thereof, a transcript thereof, or a variant or a derivative thereof. None of the previously known reports show that change in the expression of the gene or the transcript thereof can serve as a marker for breast cancer.

The 188th target gene is the hsa-miR-486-3p gene, a congener thereof, a transcript thereof, or a variant or a derivative thereof. None of the previously known reports show that change in the expression of the gene or the transcript thereof can serve as a marker for breast cancer.

The 189th target gene is the hsa-miR-6085 gene, a congener thereof, a transcript thereof, or a variant or a derivative thereof. None of the previously known reports show that change in the expression of the gene or the transcript thereof can serve as a marker for breast cancer.

The 190th target gene is the hsa-miR-4746-3p gene, a congener thereof, a transcript thereof, or a variant or a derivative thereof. None of the previously known reports show that change in the expression of the gene or the transcript thereof can serve as a marker for breast cancer.

The 191st target gene is the hsa-miR-619-5p gene, a congener thereof, a transcript thereof, or a variant or a derivative thereof. None of the previously known reports show that change in the expression of the gene or the transcript thereof can serve as a marker for breast cancer.

The 192nd target gene is the hsa-miR-937-5p gene, a congener thereof, a transcript thereof, or a variant or a derivative thereof. None of the previously known reports show that change in the expression of the gene or the transcript thereof can serve as a marker for breast cancer.

The 193rd target gene is the hsa-miR-6803-5p gene, a congener thereof, a transcript thereof, or a variant or a derivative thereof. None of the previously known reports show that change in the expression of the gene or the transcript thereof can serve as a marker for breast cancer.

The 194th target gene is the hsa-miR-4298 gene, a congener thereof, a transcript thereof, or a variant or a derivative thereof. None of the previously known reports show that change in the expression of the gene or the transcript thereof can serve as a marker for breast cancer.

The 195th target gene is the hsa-miR-4454 gene, a congener thereof, a transcript thereof, or a variant or a derivative thereof. None of the previously known reports show that change in the expression of the gene or the transcript thereof can serve as a marker for breast cancer.

The 196th target gene is the hsa-miR-4459 gene, a congener thereof, a transcript thereof, or a variant or a derivative thereof. None of the previously known reports show that change in the expression of the gene or the transcript thereof can serve as a marker for breast cancer.

The 197th target gene is the hsa-miR-7150 gene, a congener thereof, a transcript thereof, or a variant or a derivative thereof. None of the previously known reports show that change in the expression of the gene or the transcript thereof can serve as a marker for breast cancer.

The 198th target gene is the hsa-miR-6880-5p gene, a congener thereof, a transcript thereof, or a variant or a derivative thereof. None of the previously known reports show that change in the expression of the gene or the transcript thereof can serve as a marker for breast cancer.

The 199th target gene is the hsa-miR-4449 gene, a congener thereof, a transcript thereof, or a variant or a derivative thereof. None of the previously known reports show that change in the expression of the gene or the transcript thereof can serve as a marker for breast cancer.

The 200th target gene is the hsa-miR-8063 gene, a congener thereof, a transcript thereof, or a variant or a derivative thereof. None of the previously known reports show that change in the expression of the gene or the transcript thereof can serve as a marker for breast cancer.

The 201st target gene is the hsa-miR-4695-5p gene, a congener thereof, a transcript thereof, or a variant or a derivative thereof. None of the previously known reports show that change in the expression of the gene or the transcript thereof can serve as a marker for breast cancer.

The 202nd target gene is the hsa-miR-6132 gene, a congener thereof, a transcript thereof, or a variant or a derivative thereof. None of the previously known reports show that change in the expression of the gene or the transcript thereof can serve as a marker for breast cancer.

The 203rd target gene is the hsa-miR-6829-5p gene, a congener thereof, a transcript thereof, or a variant or a derivative thereof. None of the previously known reports show that change in the expression of the gene or the transcript thereof can serve as a marker for breast cancer.

The 204th target gene is the hsa-miR-4486 gene, a congener thereof, a transcript thereof, or a variant or a derivative thereof. None of the previously known reports show that change in the expression of the gene or the transcript thereof can serve as a marker for breast cancer.

The 205th target gene is the hsa-miR-6805-3p gene, a congener thereof, a transcript thereof, or a variant or a derivative thereof. None of the previously known reports show that change in the expression of the gene or the transcript thereof can serve as a marker for breast cancer.

The 206th target gene is the hsa-miR-6826-5p gene, a congener thereof, a transcript thereof, or a variant or a derivative thereof. None of the previously known reports show that change in the expression of the gene or the transcript thereof can serve as a marker for breast cancer.

The 207th target gene is the hsa-miR-4508 gene, a congener thereof, a transcript thereof, or a variant or a derivative thereof. None of the previously known reports show that change in the expression of the gene or the transcript thereof can serve as a marker for breast cancer.

The 208th target gene is the hsa-miR-1343-5p gene, a congener thereof, a transcript thereof, or a variant or a derivative thereof. None of the previously known reports show that change in the expression of the gene or the transcript thereof can serve as a marker for breast cancer.

The 209th target gene is the hsa-miR-7114-5p gene, a congener thereof, a transcript thereof, or a variant or a derivative thereof. None of the previously known reports show that change in the expression of the gene or the transcript thereof can serve as a marker for breast cancer.

The 210th target gene is the hsa-miR-3622a-5p gene, a congener thereof, a transcript thereof, or a variant or a derivative thereof. None of the previously known reports show that change in the expression of the gene or the transcript thereof can serve as a marker for breast cancer.

The 211th target gene is the hsa-miR-6765-5p gene, a congener thereof, a transcript thereof, or a variant or a derivative thereof. None of the previously known reports show that change in the expression of the gene or the transcript thereof can serve as a marker for breast cancer.

The 212th target gene is the hsa-miR-7845-5p gene, a congener thereof, a transcript thereof, or a variant or a derivative thereof. None of the previously known reports show that change in the expression of the gene or the transcript thereof can serve as a marker for breast cancer.

The 213th target gene is the hsa-miR-3960 gene, a congener thereof, a transcript thereof, or a variant or a derivative thereof. None of the previously known reports show that change in the expression of the gene or the transcript thereof can serve as a marker for breast cancer.

The 214th target gene is the hsa-miR-6749-5p gene, a congener thereof, a transcript thereof, or a variant or a derivative thereof. None of the previously known reports show that change in the expression of the gene or the transcript thereof can serve as a marker for breast cancer.

The 215th target gene is the hsa-miR-1260b gene, a congener thereof, a transcript thereof, or a variant or a derivative thereof. None of the previously known reports show that change in the expression of the gene or the transcript thereof can serve as a marker for breast cancer.

The 216th target gene is the hsa-miR-6799-5p gene, a congener thereof, a transcript thereof, or a variant or a derivative thereof. None of the previously known reports show that change in the expression of the gene or the transcript thereof can serve as a marker for breast cancer.

The 217th target gene is the hsa-miR-4723-5p gene, a congener thereof, a transcript thereof, or a variant or a derivative thereof. None of the previously known reports show that change in the expression of the gene or the transcript thereof can serve as a marker for breast cancer.

The 218th target gene is the hsa-miR-6784-5p gene, a congener thereof, a transcript thereof, or a variant or a derivative thereof. None of the previously known reports show that change in the expression of the gene or the transcript thereof can serve as a marker for breast cancer.

The 219th target gene is the hsa-miR-5100 gene, a congener thereof, a transcript thereof, or a variant or a derivative thereof. None of the previously known reports show that change in the expression of the gene or the transcript thereof can serve as a marker for breast cancer.

The 220th target gene is the hsa-miR-6769b-5p gene, a congener thereof, a transcript thereof, or a variant or a derivative thereof. None of the previously known reports show that change in the expression of the gene or the transcript thereof can serve as a marker for breast cancer.

The 221st target gene is the hsa-miR-1207-5p gene, a congener thereof, a transcript thereof, or a variant or a derivative thereof. None of the previously known reports show that change in the expression of the gene or the transcript thereof can serve as a marker for breast cancer.

The 222nd target gene is the hsa-miR-642a-3p gene, a congener thereof, a transcript thereof, or a variant or a derivative thereof. None of the previously known reports show that change in the expression of the gene or the transcript thereof can serve as a marker for breast cancer.

The 223rd target gene is the hsa-miR-4505 gene, a congener thereof, a transcript thereof, or a variant or a derivative thereof. None of the previously known reports show that change in the expression of the gene or the transcript thereof can serve as a marker for breast cancer.

The 224th target gene is the hsa-miR-4270 gene, a congener thereof, a transcript thereof, or a variant or a derivative thereof. None of the previously known reports show that change in the expression of the gene or the transcript thereof can serve as a marker for breast cancer.

The 225th target gene is the hsa-miR-6721-5p gene, a congener thereof, a transcript thereof, or a variant or a derivative thereof. None of the previously known reports show that change in the expression of the gene or the transcript thereof can serve as a marker for breast cancer.

The 226th target gene is the hsa-miR-7111-5p gene, a congener thereof, a transcript thereof, or a variant or a derivative thereof. None of the previously known reports show that change in the expression of the gene or the transcript thereof can serve as a marker for breast cancer.

The 227th target gene is the hsa-miR-6791-5p gene, a congener thereof, a transcript thereof, or a variant or a derivative thereof. None of the previously known reports show that change in the expression of the gene or the transcript thereof can serve as a marker for breast cancer.

The 228th target gene is the hsa-miR-7109-5p gene, a congener thereof, a transcript thereof, or a variant or a derivative thereof. None of the previously known reports show that change in the expression of the gene or the transcript thereof can serve as a marker for breast cancer.

The 229th target gene is the hsa-miR-4258 gene, a congener thereof, a transcript thereof, or a variant or a derivative thereof. None of the previously known reports show that change in the expression of the gene or the transcript thereof can serve as a marker for breast cancer.

The 230th target gene is the hsa-miR-6515-3p gene, a congener thereof, a transcript thereof, or a variant or a derivative thereof. None of the previously known reports show that change in the expression of the gene or the transcript thereof can serve as a marker for breast cancer.

The 231st target gene is the hsa-miR-6851-5p gene, a congener thereof, a transcript thereof, or a variant or a derivative thereof. None of the previously known reports show that change in the expression of the gene or the transcript thereof can serve as a marker for breast cancer.

The 232nd target gene is the hsa-miR-6125 gene, a congener thereof, a transcript thereof, or a variant or a derivative thereof. None of the previously known reports show that change in the expression of the gene or the transcript thereof can serve as a marker for breast cancer.

The 233rd target gene is the hsa-miR-4749-5p gene, a congener thereof, a transcript thereof, or a variant or a derivative thereof. None of the previously known reports show that change in the expression of the gene or the transcript thereof can serve as a marker for breast cancer.

The 234th target gene is the hsa-miR-4726-5p gene, a congener thereof, a transcript thereof, or a variant or a derivative thereof. None of the previously known reports show that change in the expression of the gene or the transcript thereof can serve as a marker for breast cancer.

The 235th target gene is the hsa-miR-4513 gene, a congener thereof, a transcript thereof, or a variant or a derivative thereof. None of the previously known reports show that change in the expression of the gene or the transcript thereof can serve as a marker for breast cancer.

The 236th target gene is the hsa-miR-760 gene, a congener thereof, a transcript thereof, or a variant or a derivative thereof. The previously known report shows that change in the expression of the gene or the transcript thereof can serve as a marker for breast cancer (Non-Patent Literature 3 described above).

The 237th target gene is the hsa-miR-602 gene, a congener thereof, a transcript thereof, or a variant or a derivative thereof. The previously known report shows that change in the expression of the gene or the transcript thereof can serve as a marker for breast cancer (Patent Literature 1 described above).

The 238th target gene is the hsa-miR-423-5p gene, a congener thereof, a transcript thereof, or a variant or a derivative thereof. The previously known report shows that change in the expression of the gene or the transcript thereof can serve as a marker for breast cancer (Non-Patent Literature 4 described above).

The 239th target gene is the hsa-miR-92a-2-5p gene, a congener thereof, a transcript thereof, or a variant or a derivative thereof. The previously known report shows that change in the expression of the gene or the transcript thereof can serve as a marker for breast cancer (Patent Literature 3 described above).

The 240th target gene is the hsa-miR-16-5p gene, a congener thereof, a transcript thereof, or a variant or a derivative thereof. The previously known report shows that change in the expression of the gene or the transcript thereof can serve as a marker for breast cancer (Patent Literature 4 described above).

The 241st target gene is the hsa-miR-451a gene, a congener thereof, a transcript thereof, or a variant or a derivative thereof. The previously known report shows that change in the expression of the gene or the transcript thereof can serve as a marker for breast cancer (Patent Literature 4 described above).

The 242nd target gene is the hsa-miR-135a-3p gene, a congener thereof, a transcript thereof, or a variant or a derivative thereof. The previously known report shows that change in the expression of the gene or the transcript thereof can serve as a marker for breast cancer (Patent Literatures 1 and 2 described above).

The 243rd target gene is the hsa-miR-486-5p gene, a congener thereof, a transcript thereof, or a variant or a derivative thereof. The previously known report shows that change in the expression of the gene or the transcript thereof can serve as a marker for breast cancer (Non-Patent Literature 4 described above).

The 244th target gene is the hsa-miR-4257 gene, a congener thereof, a transcript thereof, or a variant or a derivative thereof. The previously known report shows that change in the expression of the gene or the transcript thereof can serve as a marker for breast cancer (Non-Patent Literature 5 described above).

The 245th target gene is the hsa-miR-92b-5p gene, a congener thereof, a transcript thereof, or a variant or a derivative thereof. The previously known report shows that change in the expression of the gene or the transcript thereof can serve as a marker for breast cancer (Patent Literature 3 described above).

The 246th target gene is the hsa-miR-1915-3p gene, a congener thereof, a transcript thereof, or a variant or a derivative thereof. The previously known report shows that change in the expression of the gene or the transcript thereof can serve as a marker for breast cancer (Non-Patent Literature 5 described above).

The 247th target gene is the hsa-miR-718 gene, a congener thereof, a transcript thereof, or a variant or a derivative thereof. The previously known report shows that change in the expression of the gene or the transcript thereof can serve as a marker for breast cancer (Non-Patent Literature 5 described above).

The 248th target gene is the hsa-miR-940 gene, a congener thereof, a transcript thereof, or a variant or a derivative thereof. The previously known report shows that change in the expression of the gene or the transcript thereof can serve as a marker for breast cancer (Non-Patent Literature 6 described above).

The 249th target gene is the hsa-miR-296-5p gene, a congener thereof, a transcript thereof, or a variant or a derivative thereof. The previously known report shows that change in the expression of the gene or the transcript thereof can serve as a marker for breast cancer (Patent Literature 4 described above).

The 250th target gene is the hsa-miR-23b-3p gene, a congener thereof, a transcript thereof, or a variant or a derivative thereof. The previously known report shows that change in the expression of the gene or the transcript thereof can serve as a marker for breast cancer (Patent Literature 2 described above).

The 251st target gene is the hsa-miR-92a-3p gene, a congener thereof, a transcript thereof, or a variant or a derivative thereof. The previously known report shows that change in the expression of the gene or the transcript thereof can serve as a marker for breast cancer (Patent Literature 3 described above).

The 252nd target gene is the hsa-miR-658 gene, a congener thereof, a transcript thereof, or a variant or a derivative thereof. None of the previously known reports show that change in the expression of the gene or the transcript thereof can serve as a marker for breast cancer.

The 253rd target gene is the hsa-miR-6842-5p gene, a congener thereof, a transcript thereof, or a variant or a derivative thereof. None of the previously known reports show that change in the expression of the gene or the transcript thereof can serve as a marker for breast cancer.

The 254th target gene is the hsa-miR-6124 gene, a congener thereof, a transcript thereof, or a variant or a derivative thereof. None of the previously known reports show that change in the expression of the gene or the transcript thereof can serve as a marker for breast cancer.

The 255th target gene is the hsa-miR-6765-3p gene, a congener thereof, a transcript thereof, or a variant or a derivative thereof. None of the previously known reports show that change in the expression of the gene or the transcript thereof can serve as a marker for breast cancer.

The 256th target gene is the hsa-miR-7106-5p gene, a congener thereof, a transcript thereof, or a variant or a derivative thereof. None of the previously known reports show that change in the expression of the gene or the transcript thereof can serve as a marker for breast cancer.

The 257th target gene is the hsa-miR-4534 gene, a congener thereof, a transcript thereof, or a variant or a derivative thereof. None of the previously known reports show that change in the expression of the gene or the transcript thereof can serve as a marker for breast cancer.

The 258th target gene is the hsa-miR-92b-3p gene, a congener thereof, a transcript thereof, or a variant or a derivative thereof. None of the previously known reports show that change in the expression of the gene or the transcript thereof can serve as a marker for breast cancer.

The 259th target gene is the hsa-miR-3135b gene, a congener thereof, a transcript thereof, or a variant or a derivative thereof. None of the previously known reports show that change in the expression of the gene or the transcript thereof can serve as a marker for breast cancer.

The 260th target gene is the hsa-miR-4687-3p gene, a congener thereof, a transcript thereof, or a variant or a derivative thereof. None of the previously known reports show that change in the expression of the gene or the transcript thereof can serve as a marker for breast cancer.

The 261st target gene is the hsa-miR-762 gene, a congener thereof, a transcript thereof, or a variant or a derivative thereof. None of the previously known reports show that change in the expression of the gene or the transcript thereof can serve as a marker for breast cancer.

The 262nd target gene is the hsa-miR-3619-3p gene, a congener thereof, a transcript thereof, or a variant or a derivative thereof. None of the previously known reports show that change in the expression of the gene or the transcript thereof can serve as a marker for breast cancer.

The 263rd target gene is the hsa-miR-4467 gene, a congener thereof, a transcript thereof, or a variant or a derivative thereof. None of the previously known reports show that change in the expression of the gene or the transcript thereof can serve as a marker for breast cancer.

The 264th target gene is the hsa-miR-557 gene, a congener thereof, a transcript thereof, or a variant or a derivative thereof. None of the previously known reports show that change in the expression of the gene or the transcript thereof can serve as a marker for breast cancer.

The 265th target gene is the hsa-miR-1237-5p gene, a congener thereof, a transcript thereof, or a variant or a derivative thereof. None of the previously known reports show that change in the expression of the gene or the transcript thereof can serve as a marker for breast cancer.

The 266th target gene is the hsa-miR-1908-5p gene, a congener thereof, a transcript thereof, or a variant or a derivative thereof. None of the previously known reports show that change in the expression of the gene or the transcript thereof can serve as a marker for breast cancer.

The 267th target gene is the hsa-miR-4286 gene, a congener thereof, a transcript thereof, or a variant or a derivative thereof. None of the previously known reports show that change in the expression of the gene or the transcript thereof can serve as a marker for breast cancer.

The 268th target gene is the hsa-miR-6885-5p gene, a congener thereof, a transcript thereof, or a variant or a derivative thereof. None of the previously known reports show that change in the expression of the gene or the transcript thereof can serve as a marker for breast cancer.

The 269th target gene is the hsa-miR-6763-5p gene, a congener thereof, a transcript thereof, or a variant or a derivative thereof. None of the previously known reports show that change in the expression of the gene or the transcript thereof can serve as a marker for breast cancer.

The 270th target gene is the hsa-miR-6089 gene, a congener thereof, a transcript thereof, or a variant or a derivative thereof. None of the previously known reports show that change in the expression of the gene or the transcript thereof can serve as a marker for breast cancer.

The 271st target gene is the hsa-miR-6816-5p gene, a congener thereof, a transcript thereof, or a variant or a derivative thereof. None of the previously known reports show that change in the expression of the gene or the transcript thereof can serve as a marker for breast cancer.

The 272nd target gene is the hsa-miR-4466 gene, a congener thereof, a transcript thereof, or a variant or a derivative thereof. None of the previously known reports show that change in the expression of the gene or the transcript thereof can serve as a marker for breast cancer.

The 273rd target gene is the hsa-miR-4488 gene, a congener thereof, a transcript thereof, or a variant or a derivative thereof. None of the previously known reports show that change in the expression of the gene or the transcript thereof can serve as a marker for breast cancer.

The 274th target gene is the hsa-miR-6752-5p gene, a congener thereof, a transcript thereof, or a variant or a derivative thereof. None of the previously known reports show that change in the expression of the gene or the transcript thereof can serve as a marker for breast cancer.

The 275th target gene is the hsa-miR-4739 gene, a congener thereof, a transcript thereof, or a variant or a derivative thereof. None of the previously known reports show that change in the expression of the gene or the transcript thereof can serve as a marker for breast cancer.

2. Nucleic Acid Probe or Primer for Detection of Breast Cancer

In the present invention, nucleic acid(s) capable of specifically binding to any of the target nucleic acids as the breast cancer markers described above can be used as a nucleic acid, for example, a nucleic acid probe or a primer, for the detection or diagnosis of breast cancer.

In the present invention, the nucleic acid probes or the primers that can be used for detecting breast cancer or for diagnosing breast cancer enables qualitative and/or quantitative measurement of the presence, expression level, or existing amount (abundance) of any of the target nucleic acids as the breast cancer markers described above, for example, human-derived hsa-miR-4783-3p, hsa-miR-4730, hsa-miR-1307-3p, hsa-miR-4634, hsa-miR-663a, hsa-miR-4532, hsa-miR-7704, hsa-miR-3178, hsa-miR-6729-5p, hsa-miR-6090, hsa-miR-4732-5p, hsa-miR-3184-5p, hsa-miR-6727-5p, hsa-miR-6088, hsa-miR-4674, hsa-miR-8073, hsa-miR-4787-5p, hsa-miR-1469, hsa-miR-125a-3p, hsa-miR-1233-5p, hsa-miR-885-3p, hsa-miR-6802-5p, hsa-miR-328-5p, hsa-miR-6787-5p, hsa-miR-8069, hsa-miR-6875-5p, hsa-miR-1246, hsa-miR-4734, hsa-miR-6757-5p, hsa-miR-6756-5p, hsa-miR-3665, hsa-miR-6836-3p, hsa-miR-6821-5p, hsa-miR-6805-5p, hsa-miR-4728-5p, hsa-miR-6726-5p, hsa-miR-197-5p, hsa-miR-149-3p, hsa-miR-6850-5p, hsa-miR-4476, hsa-miR-6858-5p, hsa-miR-564, hsa-miR-4763-3p, hsa-miR-575, hsa-miR-6771-5p, hsa-miR-1231, hsa-miR-1908-3p, hsa-miR-150-3p, hsa-miR-3937, hsa-miR-887-3p, hsa-miR-3940-5p, hsa-miR-4741, hsa-miR-6808-5p, hsa-miR-6869-5p, hsa-miR-5090, hsa-miR-615-5p, hsa-miR-8072, hsa-miR-128-1-5p, hsa-miR-1238-5p, hsa-miR-365a-5p, hsa-miR-204-3p, hsa-miR-4492, hsa-miR-6785-5p, hsa-miR-6511a-5p, hsa-miR-4525, hsa-miR-1915-5p, hsa-miR-3180, hsa-miR-6879-5p, hsa-miR-1199-5p, hsa-miR-6746-5p, hsa-miR-711, hsa-miR-663b, hsa-miR-4707-3p, hsa-miR-6893-5p, hsa-miR-4675, hsa-miR-4638-5p, hsa-miR-4651, hsa-miR-6087, hsa-miR-4665-5p, hsa-miR-4758-5p, hsa-miR-6887-5p, hsa-miR-3620-5p, hsa-miR-1909-3p, hsa-miR-7641, hsa-miR-6724-5p, hsa-miR-1343-3p, hsa-miR-6780b-5p, hsa-miR-4484, hsa-miR-4690-5p, hsa-miR-4429, hsa-miR-1227-5p, hsa-miR-4725-3p, hsa-miR-6861-5p, hsa-miR-6812-5p, hsa-miR-3197,hsa-miR-8059,hsa-miR-3185,hsa-miR-4706,hsa-miR-4497, hsa-miR-3131, hsa-miR-6806-5p, hsa-miR-187-5p, hsa-miR-3180-3p, hsa-miR-6848-5p, hsa-miR-6820-5p, hsa-miR-6800-5p, hsa-miR-6717-5p, hsa-miR-6795-5p, hsa-miR-4632-5p, hsa-miR-665, hsa-miR-6778-5p, hsa-miR-3663-3p, hsa-miR-4689, hsa-miR-211-3p, hsa-miR-6511b-5p, hsa-miR-4750-5p, hsa-miR-6126, hsa-miR-614, hsa-miR-7110-5p, hsa-miR-744-5p, hsa-miR-6769a-5p, hsa-miR-4792, hsa-miR-5787, hsa-miR-6798-5p, hsa-miR-6781-5p, hsa-miR-4419b, hsa-miR-4446-3p, hsa-miR-4259, hsa-miR-5572, hsa-miR-6075, hsa-miR-296-3p, hsa-miR-6891-5p, hsa-miR-4745-5p, hsa-miR-6775-5p, hsa-miR-6870-5p, hsa-miR-920, hsa-miR-4530, hsa-miR-6819-5p, hsa-miR-6825-5p, hsa-miR-7847-3p, hsa-miR-6131, hsa-miR-4433-3p, hsa-miR-1228-5p, hsa-miR-6743-5p, hsa-miR-1268a, hsa-miR-3917, hsa-miR-6786-5p, hsa-miR-3154, hsa-miR-638, hsa-miR-6741-5p, hsa-miR-6889-5p, hsa-miR-6840-3p, hsa-miR-6510-5p, hsa-miR-3188, hsa-miR-551b-5p, hsa-miR-5001-5p, hsa-miR-1268b, hsa-miR-7107-5p, hsa-miR-6824-5p, hsa-miR-6732-5p, hsa-miR-371a-5p, hsa-miR-6794-5p, hsa-miR-6779-5p, hsa-miR-4271, hsa-miR-5195-3p, hsa-miR-6762-5p, hsa-miR-939-5p, hsa-miR-1247-3p, hsa-miR-6777-5p, hsa-miR-6722-3p, hsa-miR-3656, hsa-miR-4688, hsa-miR-3195, hsa-miR-6766-5p, hsa-miR-4447, hsa-miR-4656, hsa-miR-7108-5p, hsa-miR-3191-3p, hsa-miR-1273g-3p, hsa-miR-4463, hsa-miR-2861, hsa-miR-3196, hsa-miR-6877-5p, hsa-miR-3679-5p, hsa-miR-4442, hsa-miR-6789-5p, hsa-miR-6782-5p, hsa-miR-486-3p, hsa-miR-6085, hsa-miR-4746-3p, hsa-miR-619-5p, hsa-miR-937-5p, hsa-miR-6803-5p, hsa-miR-4298, hsa-miR-4454, hsa-miR-4459, hsa-miR-7150, hsa-miR-6880-5p, hsa-miR-4449, hsa-miR-8063, hsa-miR-4695-5p, hsa-miR-6132, hsa-miR-6829-5p, hsa-miR-4486, hsa-miR-6805-3p, hsa-miR-6826-5p, hsa-miR-4508, hsa-miR-1343-5p, hsa-miR-7114-5p, hsa-miR-3622a-5p, hsa-miR-6765-5p, hsa-miR-7845-5p, hsa-miR-3960, hsa-miR-6749-5p, hsa-miR-1260b, hsa-miR-6799-5p, hsa-miR-4723-5p, hsa-miR-6784-5p, hsa-miR-5100, hsa-miR-6769b-5p, hsa-miR-1207-5p, hsa-miR-642a-3p, hsa-miR-4505, hsa-miR-4270, hsa-miR-6721-5p, hsa-miR-7111-5p, hsa-miR-6791-5p, hsa-miR-7109-5p, hsa-miR-4258, hsa-miR-6515-3p, hsa-miR-6851-5p, hsa-miR-6125, hsa-miR-4749-5p, hsa-miR-4726-5p, hsa-miR-4513, hsa-miR-6089, hsa-miR-6816-5p, hsa-miR-4466, hsa-miR-4488, hsa-miR-6752-5p and hsa-miR-4739 or a combination thereof, congeners thereof, transcripts thereof, or variants or derivatives thereof; and, optionally in combination therewith, hsa-miR-760, hsa-miR-602, hsa-miR-423-5p, hsa-miR-92a-2-5p, hsa-miR-16-5p, hsa-miR-451a, hsa-miR-135a-3p, hsa-miR-486-5p, hsa-miR-4257, hsa-miR-92b-5p, hsa-miR-1915-3p, hsa-miR-718, hsa-miR-940, hsa-miR-296-5p, hsa-miR-23b-3p and hsa-miR-92a-3p or a combination thereof, transcripts thereof, or variants or derivatives thereof; and, optionally in combination therewith, hsa-miR-658, hsa-miR-6842-5p, hsa-miR-6124, hsa-miR-6765-3p, hsa-miR-7106-5p, hsa-miR-4534, hsa-miR-92b-3p, hsa-miR-3135b, hsa-miR-4687-3p, hsa-miR-762, hsa-miR-3619-3p, hsa-miR-4467, hsa-miR-557, hsa-miR-1237-5p, hsa-miR-1908-5p, hsa-miR-4286, hsa-miR-6885-5p and hsa-miR-6763-5p or a combination thereof; congeners thereof; transcripts thereof; or variants or derivatives thereof.

The expression level of the target nucleic acids described above are increased or decreased (hereinafter, referred to as “increased/decreased”) depending on the types of the target nucleic acids in subjects who have breast cancer, as compared with healthy subjects. Hence, the nucleic acid of the present invention can be effectively used for measuring expression levels of the target nucleic acids in body fluids derived from subjects (e.g., humans) suspected of having breast cancer and body fluids derived from healthy subjects and thereby detecting breast cancer through the comparison thereof.

The nucleic acid probe or the primer that can be used in the present invention is a nucleic acid probe capable of specifically binding to a polynucleotide consisting of a nucleotide sequence represented by at least one of SEQ ID NOs: 1 to 235 and 851 to 856, or a primer for amplifying a polynucleotide consisting of a nucleotide sequence represented by at least one of SEQ ID NOs: 1 to 235 and 851 to 856.

The nucleic acid probe or the primer that can be used in the present invention may further comprise a nucleic acid probe capable of specifically binding to a polynucleotide consisting of a nucleotide sequence represented by at least one of SEQ ID NOs: 236 to 251, or a primer for amplifying a polynucleotide consisting of a nucleotide sequence represented by at least one of SEQ ID NOs: 236 to 251.

The nucleic acid probe or the primer that can be used in the present invention may further comprise a nucleic acid probe capable of specifically binding to a polynucleotide that consists of a nucleotide sequence represented by at least one of SEQ ID NOs: 252 to 269, or a primer for amplifying a polynucleotide that consists of a nucleotide sequence represented by at least one of SEQ ID NOs: 252 to 269.

Specifically, these nucleic acid probes or primers comprise a combination of one or more polynucleotides selected from: a group of polynucleotides that comprise nucleotide sequences represented by any of SEQ ID NOs: 1 to 871 or nucleotide sequences derived from the nucleotide sequences by the replacement of u with t, and a group of complementary polynucleotides thereof, a group of polynucleotides that respectively hybridize under stringent conditions (mentioned later) to DNAs consisting of nucleotide sequences complementary to these nucleotide sequences; and a group of complementary polynucleotides thereof, and a group of polynucleotide comprising 15 or more, preferably 17 or more consecutive nucleotides in the nucleotide sequences of these polynucleotides. These polynucleotides can be used as nucleic acid probes and primers for detecting the breast cancer markers as target nucleic acids.

More specifically, examples of the nucleic acid probes or the primers that can be used in the present invention include one or more polynucleotides selected from the group consisting of the following polynucleotides (a) to (e):
(a) a polynucleotide consisting of a nucleotide sequence represented by any of SEQ ID NOs: 1 to 235, and 851 to 856 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence complementary to a nucleotide sequence represented by any of SEQ ID NOs: 1 to 235, and 851 to 856 or a nucleotide sequence derived from the nucleotide sequence by the replacement of u with t,
(c) a polynucleotide consisting of a nucleotide sequence complementary to a nucleotide sequence represented by any of SEQ ID NOs: 1 to 235, and 851 to 856 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 235, and 851 to 856 or a nucleotide sequence derived from the nucleotide sequence by the replacement of u with t, and
(e) a polynucleotide hybridizing under stringent conditions to any of the polynucleotides (a) to (d).

In addition to at least one polynucleotide selected from the group consisting of the polynucleotides (a) to (e), the nucleic acid probes or the primers that can be used in the present invention may further comprise a polynucleotide selected from the group consisting of the following polynucleotides (f) to (j):
(f) a polynucleotide consisting of a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 236 to 251,
(h) a polynucleotide consisting of a nucleotide sequence complementary to a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 236 to 251 or a nucleotide sequence derived from the nucleotide sequence by the replacement of u with t, and
(j) a polynucleotide hybridizing under stringent conditions to any of the polynucleotides (f) to (i).

In addition to at least one polynucleotide selected from the group consisting of (a) to (j) described above, the nucleic acid probes or the primesr that can be used in the present invention may further comprise a polynucleotide selected from the group consisting of the following polynucleotides (k) to (o):
(k) a polynucleotide consisting of a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 252 to 269,
(m) a polynucleotide consisting of a nucleotide sequence complementary to a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 252 to 269 or a nucleotide sequence derived from the nucleotide sequence by the replacement of u with t, and
(o) a polynucleotide hybridizing under stringent conditions to any of the polynucleotides (k) to (n).

For these polynucleotides, the “fragment thereof comprising 15 or more consecutive nucleotides” can comprise, but is not limited to, the number of nucleotides in the range from, for example, 15 consecutive nucleotides to less than the total number of nucleotides of the sequence, from 17 consecutive nucleotides to less than the total number of nucleotides of the sequence, or from 19 consecutive nucleotides to less than the total number of nucleotides of the sequence, in the nucleotide sequence of each polynucleotide.

These polynucleotides or fragments thereof used in the present invention may each be DNA or may each be RNA.

The polynucleotides that can be used in the present invention can each be prepared by use of a general technique such as a DNA recombination technique, a PCR method, or a method using an automatic DNA/RNA synthesizer.

The DNA recombination technique and the PCR method may employ techniques described in, for example, Ausubel et al., Current Protocols in Molecular Biology, John Willey & Sons, US (1993); and Sambrook et al., Molecular Cloning—A Laboratory Manual, Cold Spring Harbor Laboratory Press, US (1989).

The human-derived hsa-miR-4783-3p, hsa-miR-4730, hsa-miR-1307-3p, hsa-miR-4634, hsa-miR-663a, hsa-miR-4532, hsa-miR-7704, hsa-miR-3178, hsa-miR-6729-5p, hsa-miR-6090, hsa-miR-4732-5p, hsa-miR-3184-5p, hsa-miR-6727-5p, hsa-miR-6088, hsa-miR-4674, hsa-miR-8073, hsa-miR-4787-5p, hsa-miR-1469, hsa-miR-125a-3p, hsa-miR-1233-5p, hsa-miR-885-3p, hsa-miR-6802-5p, hsa-miR-328-5p, hsa-miR-6787-5p, hsa-miR-8069, hsa-miR-6875-5p, hsa-miR-1246, hsa-miR-4734, hsa-miR-6757-5p, hsa-miR-6756-5p, hsa-miR-3665, hsa-miR-6836-3p, hsa-miR-6821-5p, hsa-miR-6805-5p, hsa-miR-4728-5p, hsa-miR-6726-5p, hsa-miR-197-5p, hsa-miR-149-3p, hsa-miR-6850-5p, hsa-miR-4476, hsa-miR-6858-5p, hsa-miR-564, hsa-miR-4763-3p, hsa-miR-575, hsa-miR-6771-5p, hsa-miR-1231, hsa-miR-1908-3p, hsa-miR-150-3p, hsa-miR-3937, hsa-miR-887-3p, hsa-miR-3940-5p, hsa-miR-4741, hsa-miR-6808-5p, hsa-miR-6869-5p, hsa-miR-5090, hsa-miR-615-5p, hsa-miR-8072, hsa-miR-128-1-5p, hsa-miR-1238-5p, hsa-miR-365a-5p, hsa-miR-204-3p, hsa-miR-4492, hsa-miR-6785-5p, hsa-miR-6511a-5p, hsa-miR-4525, hsa-miR-1915-5p, hsa-miR-3180, hsa-miR-6879-5p, hsa-miR-1199-5p, hsa-miR-6746-5p, hsa-miR-711, hsa-miR-663b, hsa-miR-4707-3p, hsa-miR-6893-5p, hsa-miR-4675, hsa-miR-4638-5p, hsa-miR-4651, hsa-miR-6087, hsa-miR-4665-5p, hsa-miR-4758-5p, hsa-miR-6887-5p, hsa-miR-3620-5p, hsa-miR-1909-3p, hsa-miR-7641, hsa-miR-6724-5p, hsa-miR-1343-3p, hsa-miR-6780b-5p, hsa-miR-4484, hsa-miR-4690-5p, hsa-miR-4429, hsa-miR-1227-5p, hsa-miR-4725-3p, hsa-miR-6861-5p, hsa-miR-6812-5p, hsa-miR-3197, hsa-miR-8059, hsa-miR-3185, hsa-miR-4706, hsa-miR-4497, hsa-miR-3131, hsa-miR-6806-5p, hsa-miR-187-5p, hsa-miR-3180-3p, hsa-miR-6848-5p, hsa-miR-6820-5p, hsa-miR-6800-5p, hsa-miR-6717-5p, hsa-miR-6795-5p, hsa-miR-4632-5p, hsa-miR-665, hsa-miR-6778-5p, hsa-miR-3663-3p, hsa-miR-4689, hsa-miR-211-3p, hsa-miR-6511b-5p, hsa-miR-4750-5p, hsa-miR-6126, hsa-miR-614, hsa-miR-7110-5p, hsa-miR-744-5p, hsa-miR-6769a-5p, hsa-miR-4792, hsa-miR-5787, hsa-miR-6798-5p, hsa-miR-6781-5p, hsa-miR-4419b, hsa-miR-4446-3p, hsa-miR-4259, hsa-miR-5572, hsa-miR-6075, hsa-miR-296-3p, hsa-miR-6891-5p, hsa-miR-4745-5p, hsa-miR-6775-5p, hsa-miR-6870-5p, hsa-miR-920, hsa-miR-4530, hsa-miR-6819-5p, hsa-miR-6825-5p, hsa-miR-7847-3p, hsa-miR-6131, hsa-miR-4433-3p, hsa-miR-1228-5p, hsa-miR-6743-5p, hsa-miR-1268a, hsa-miR-3917, hsa-miR-6786-5p, hsa-miR-3154, hsa-miR-638, hsa-miR-6741-5p, hsa-miR-6889-5p, hsa-miR-6840-3p, hsa-miR-6510-5p, hsa-miR-3188, hsa-miR-551b-5p, hsa-miR-5001-5p, hsa-miR-1268b, hsa-miR-7107-5p, hsa-miR-6824-5p, hsa-miR-6732-5p, hsa-miR-371a-5p, hsa-miR-6794-5p, hsa-miR-6779-5p, hsa-miR-4271, hsa-miR-5195-3p, hsa-miR-6762-5p, hsa-miR-939-5p, hsa-miR-1247-3p, hsa-miR-6777-5p, hsa-miR-6722-3p, hsa-miR-3656, hsa-miR-4688, hsa-miR-3195, hsa-miR-6766-5p, hsa-miR-4447, hsa-miR-4656, hsa-miR-7108-5p, hsa-miR-3191-3p, hsa-miR-1273g-3p, hsa-miR-4463, hsa-miR-2861, hsa-miR-3196, hsa-miR-6877-5p, hsa-miR-3679-5p, hsa-miR-4442, hsa-miR-6789-5p, hsa-miR-6782-5p, hsa-miR-486-3p, hsa-miR-6085, hsa-miR-4746-3p, hsa-miR-619-5p, hsa-miR-937-5p, hsa-miR-6803-5p, hsa-miR-4298, hsa-miR-4454, hsa-miR-4459, hsa-miR-7150, hsa-miR-6880-5p, hsa-miR-4449, hsa-miR-8063, hsa-miR-4695-5p, hsa-miR-6132, hsa-miR-6829-5p, hsa-miR-4486, hsa-miR-6805-3p, hsa-miR-6826-5p, hsa-miR-4508, hsa-miR-1343-5p, hsa-miR-7114-5p, hsa-miR-3622a-5p, hsa-miR-6765-5p, hsa-miR-7845-5p, hsa-miR-3960, hsa-miR-6749-5p, hsa-miR-1260b, hsa-miR-6799-5p, hsa-miR-4723-5p, hsa-miR-6784-5p, hsa-miR-5100, hsa-miR-6769b-5p, hsa-miR-1207-5p, hsa-miR-642a-3p, hsa-miR-4505, hsa-miR-4270, hsa-miR-6721-5p, hsa-miR-7111-5p, hsa-miR-6791-5p, hsa-miR-7109-5p, hsa-miR-4258, hsa-miR-6515-3p, hsa-miR-6851-5p, hsa-miR-6125, hsa-miR-4749-5p, hsa-miR-4726-5p, hsa-miR-4513, hsa-miR-6089, hsa-miR-6816-5p, hsa-miR-4466, hsa-miR-4488, hsa-miR-6752-5p, hsa-miR-4739, hsa-miR-760, hsa-miR-602, hsa-miR-423-5p, hsa-miR-92a-2-5p, hsa-miR-16-5p, hsa-miR-451a, hsa-miR-135a-3p, hsa-miR-486-5p, hsa-miR-4257, hsa-miR-92b-5p, hsa-miR-1915-3p, hsa-miR-718, hsa-miR-940, hsa-miR-296-5p, hsa-miR-23b-3p, hsa-miR-92a-3p, hsa-miR-658, hsa-miR-6842-5p, hsa-miR-6124, hsa-miR-6765-3p, hsa-miR-7106-5p, hsa-miR-4534, hsa-miR-92b-3p, hsa-miR-3135b, hsa-miR-4687-3p, hsa-miR-762, hsa-miR-3619-3p, hsa-miR-4467, hsa-miR-557, hsa-miR-1237-5p, hsa-miR-1908-5p, hsa-miR-4286, hsa-miR-6885-5p and hsa-miR-6763-5p represented by SEQ ID NOs: 1 to 269, and 851 to 856 are known in the art, and their acquisition methods are also known as mentioned above. Therefore, each polynucleotide that can be used as a nucleic acid probe or a primer in the present invention can be prepared by cloning the gene.

Such nucleic acid probes or primers can be chemically synthesized using an automatic DNA synthesizer. In general, the phosphoramidite method is used in this synthesis, and single-stranded DNA up to approximately 100 nucleotides can be automatically synthesized by this method. The automatic DNA synthesizer is commercially available from, for example, Polygen GmbH, ABI, or Applied Biosystems, Inc.

Alternatively, the polynucleotides of the present invention can also be prepared by a cDNA cloning methods. The cDNA cloning technique may employ, for example, microRNA Cloning Kit Wako.

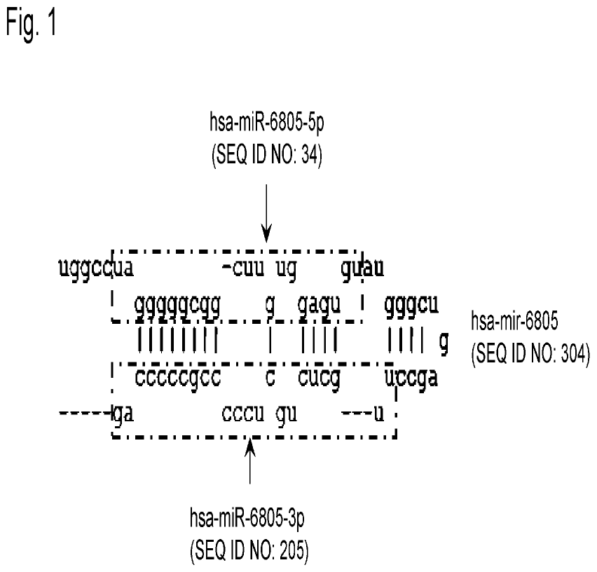

In this context, the sequences of the nucleic acid probes and the primers for detecting the polynucleotide consisting of a nucleotide sequence represented by any of SEQ ID NOs: 1 to 269, and 851 to 856 do not exist as miRNAs or precursors thereof in the living body or in vivo. For example, the nucleotide sequences represented by SEQ ID NO: 34 and SEQ ID NO: 205 are produced from the precursor represented by SEQ ID NO: 304. This precursor has a hairpin-like structure as shown in FIG. 1, and the nucleotide sequences represented by SEQ ID NO: 34 and SEQ ID NO: 205 have mismatch sequences with each other. As such, a nucleotide sequence completely complementary to the nucleotide sequence represented by SEQ ID NO: 34 or SEQ ID NO: 205 is not naturally produced in vivo. Likewise, the nucleic acid probe and the primer for detecting the nucleotide sequence represented by any of SEQ ID NOs: 1 to 269, and 851 to 856 have artificial nucleotide sequences that do not exist in the living body or in vivo.

3. Kit or Device for Detection of Breast Cancer

The present invention also provides a kit or a device for the detection of breast cancer, comprising one or more polynucleotides (which may include a variant, a fragment, or a derivative thereof; hereinafter, also referred to as polynucleotide for detection) that can be used as nucleic acid probes or primers in the present invention for measuring target nucleic acids as breast cancer markers.

The target nucleic acid as a breast cancer marker according to the present invention is selected from the following group 1:
miR-4783-3p, miR-4730, miR-1307-3p, miR-4634, miR-663a, miR-4532, miR-7704, miR-3178, miR-6729-5p, miR-6090, miR-4732-5p, miR-3184-5p, miR-6727-5p, miR-6088, miR-4674, miR-8073, miR-4787-5p, miR-1469, miR-125a-3p, miR-1233-5p, miR-885-3p, miR-6802-5p, miR-328-5p, miR-6787-5p, miR-8069, miR-6875-5p, miR-1246, miR-4734, miR-6757-5p, miR-6756-5p, miR-3665, miR-6836-3p, miR-6821-5p, miR-6805-5p, miR-4728-5p, miR-6726-5p, miR-197-5p, miR-149-3p, miR-6850-5p, miR-4476, miR-6858-5p, miR-564, miR-4763-3p, miR-575, miR-6771-5p, miR-1231, miR-1908-3p, miR-150-3p, miR-3937, miR-887-3p, miR-3940-5p, miR-4741, miR-6808-5p, miR-6869-5p, miR-5090, miR-615-5p, miR-8072, miR-128-1-5p, miR-1238-5p, miR-365a-5p, miR-204-3p, miR-4492, miR-6785-5p, miR-6511a-5p, miR-4525, miR-1915-5p, miR-3180, miR-6879-5p, miR-1199-5p, miR-6746-5p, miR-711, miR-663b, miR-4707-3p, miR-6893-5p, miR-4675, miR-4638-5p, miR-4651, miR-6087, miR-4665-5p, miR-4758-5p, miR-6887-5p, miR-3620-5p, miR-1909-3p, miR-7641, miR-6724-5p, miR-1343-3p, miR-6780b-5p, miR-4484, miR-4690-5p, miR-4429, miR-1227-5p, miR-4725-3p, miR-6861-5p, miR-6812-5p, miR-3197, miR-8059, miR-3185, miR-4706, miR-4497, miR-3131, miR-6806-5p, miR-187-5p, miR-3180-3p, miR-6848-5p, miR-6820-5p, miR-6800-5p, miR-6717-5p, miR-6795-5p, miR-4632-5p, miR-665, miR-6778-5p, miR-3663-3p, miR-4689, miR-211-3p, miR-6511b-5p, miR-4750-5p, miR-6126, miR-614, miR-7110-5p, miR-744-5p, miR-6769a-5p, miR-4792, miR-5787, miR-6798-5p, miR-6781-5p, miR-4419b, miR-4446-3p, miR-4259, miR-5572, miR-6075, miR-296-3p, miR-6891-5p, miR-4745-5p, miR-6775-5p, miR-6870-5p, miR-920, miR-4530, miR-6819-5p, miR-6825-5p, miR-7847-3p, miR-6131, miR-4433-3p, miR-1228-5p, miR-6743-5p, miR-1268a, miR-3917, miR-6786-5p, miR-3154, miR-638, miR-6741-5p, miR-6889-5p, miR-6840-3p, miR-6510-5p, miR-3188, miR-551b-5p, miR-5001-5p, miR-1268b, miR-7107-5p, miR-6824-5p, miR-6732-5p, miR-371a-5p, miR-6794-5p, miR-6779-5p, miR-4271, miR-5195-3p, miR-6762-5p, miR-939-5p, miR-1247-3p, miR-6777-5p, miR-6722-3p, miR-3656, miR-4688, miR-3195, miR-6766-5p, miR-4447, miR-4656, miR-7108-5p, miR-3191-3p, miR-1273g-3p, miR-4463, miR-2861, miR-3196, miR-6877-5p, miR-3679-5p, miR-4442, miR-6789-5p, miR-6782-5p, miR-486-3p, miR-6085, miR-4746-3p, miR-619-5p, miR-937-5p, miR-6803-5p, miR-4298, miR-4454, miR-4459, miR-7150, miR-6880-5p, miR-4449, miR-8063, miR-4695-5p, miR-6132, miR-6829-5p, miR-4486, miR-6805-3p, miR-6826-5p, miR-4508, miR-1343-5p, miR-7114-5p, miR-3622a-5p, miR-6765-5p, miR-7845-5p, miR-3960, miR-6749-5p, miR-1260b, miR-6799-5p, miR-4723-5p, miR-6784-5p, miR-5100, miR-6769b-5p, miR-1207-5p, miR-642a-3p, miR-4505, miR-4270, miR-6721-5p, miR-7111-5p, miR-6791-5p, miR-7109-5p, miR-4258, miR-6515-3p, miR-6851-5p, miR-6125, miR-4749-5p, miR-4726-5p, miR-4513, miR-6089, miR-6816-5p, miR-4466, miR-4488, miR-6752-5p and miR-4739.

An additional target nucleic acid(s) that may be optionally used in the measurement is selected from the following group 2:
miR-760, miR-602, miR-423-5p, miR-92a-2-5p, miR-16-5p, miR-451a, miR-135a-3p, miR-486-5p, miR-4257, miR-92b-5p, miR-1915-3p, miR-718, miR-940, miR-296-5p, miR-23b-3p and miR-92a-3p.

An additional target nucleic acid(s) that may be optionally used in the measurement is further selected from the following group 3:
miR-658, miR-6842-5p, miR-6124, miR-6765-3p, miR-7106-5p, miR-4534, miR-92b-3p, miR-3135b, miR-4687-3p, miR-762, miR-3619-3p, miR-4467, miR-557, miR-1237-5p, miR-1908-5p, miR-4286, miR-6885-5p and miR-6763-5p.

The kit or the device of the present invention comprises nucleic acid(s) capable of specifically binding to any of the target nucleic acids as the breast cancer markers described above, preferably one or more polynucleotides selected from the nucleic acid probes or the primers described in the preceding Section 2, specifically, the polynucleotides described in the preceding Section 2, or variants thereof, etc.

Specifically, the kit or the device of the present invention can comprise at least one polynucleotides comprising (or consisting of) a nucleotide sequence represented by any of SEQ ID NOs: 1 to 235, and 851 to 856 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, or variant(s) or fragment(s) comprising 15 or more consecutive nucleotides of any of these polynucleotide sequences.

The kit or the device of the present invention can further comprise one or more polynucleotides comprising (or consisting of) a nucleotide sequence represented by any of SEQ ID NOs: 236 to 251 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The kit or the device of the present invention can further comprise one or more polynucleotide(s) comprising (or consisting of) a nucleotide sequence represented by any of SEQ ID NOs: 252 to 269 or a nucleotide sequence derived from the nucleotide sequence by the replacement of u with t, polynucleotide(s) comprising (or consisting of) a complementary sequence thereof, polynucleotide(s) hybridizing under stringent conditions to any of these polynucleotides, variant(s) or fragment(s) comprising 15 or more consecutive nucleotides of any of these polynucleotide sequences.

The fragment(s) that can be comprised in the kit or the device of the present invention is/are, for example, one or more polynucleotides, preferably two or more polynucleotides selected from the group consisting of the following polynucleotides (1) to (3):
(1) a polynucleotide comprising 15 or more consecutive nucleotides in a nucleotide sequence that is derived from a nucleotide sequence represented by any of SEQ ID NOs: 1 to 235, and 851 to 856 by the replacement of u with t, or a complementary sequence thereof;
(2) a polynucleotide comprising 15 or more consecutive nucleotides in a nucleotide sequence that is derived from a nucleotide sequence represented by any of SEQ ID NOs: 236 to 251 by the replacement of u with t, or a complementary sequence thereof; and
(3) a polynucleotide comprising 15 or more consecutive nucleotides in a nucleotide sequence that is derived from a nucleotide sequence represented by any of SEQ ID NOs: 252 to 269 by the replacement of u with t, or a complementary sequence thereof.

In a preferred embodiment, the polynucleotide is a polynucleotide consisting of a nucleotide sequence represented by any of SEQ ID NOs: 1 to 235, and 851 to 856 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 236 to 251 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 252 to 269 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the fragment can be a polynucleotide comprising 15 or more, preferably 17 or more, more preferably 19 or more consecutive nucleotides.

In the present invention, the size of the polynucleotide fragment is the number of nucleotides in the range from, for example, 15 consecutive nucleotides to less than the total number of nucleotides of the sequence, from 17 consecutive nucleotides to less than the total number of nucleotides of the sequence, or from 19 consecutive nucleotides to less than the total number of nucleotides of the sequence, in the nucleotide sequence of each polynucleotide.

Specific examples of the aforementioned polynucleotide combination that constitutes the kit or the device of the present invention can include combinations of 1, 2, 3, 4, 5, 6, 7, 8, 9, 10 or more of the polynucleotides consisting of the nucleotide sequences represented by SEQ ID NOs: 1 to 269 and 851 to 856 shown in Table 1 described above. However, these are given merely for illustrative purposes, and all of various other possible combinations are included in the present invention.

The aforementioned combination constituting the kit or the device for discriminating a breast cancer patient from a healthy subject according to the present invention is desirably, for example, a combination of two or more polynucleotides consisting of the nucleotide sequences represented by SEQ ID NOs shown in Table 1. Usually, a combination of two of these polynucleotides can produce adequate performance. Specifically, any two of the aforementioned polynucleotides that consist of the aforementioned nucleotide sequences represented by SEQ ID NOs: 1 to 269 and 851 to 856 may be combined. For such a combination, it is preferred to select at least one of the newly found polynucleotides consisting of the nucleotide sequences represented by SEQ ID NOs: 1 to 235 and 851 to 856. More specifically, the combination is more preferably a combination comprising at least one of polynucleotides consisting of the nucleotide sequences represented by SEQ ID NOs: 20, 24, 26, 27, 30, 33, 182, 194, 206, and 208, among the combinations of the polynucleotides consisting of the nucleotide sequences represented by SEQ ID NOs: 1 to 269 and 851 to 856.

The combination of polynucleotides with cancer type specificity capable of discriminating a breast cancer patient not only from a healthy subject but also from other cancer patients is preferably, for example, a combination of multiple polynucleotides comprising at least one polynucleotide selected from the group consisting of polynucleotides consisting of the nucleotide sequences represented by SEQ ID NOs: 1, 2, 3, 4, 5, 6, 7, 8, 9, 11, 12, 13, 14, 15, 16, 17, 20, 21, 22, 23, 24, 25, 26, 27, 29, 30, 31, 32, 33, 34, 35, 36, 37, 38, 39, 41, 43, 45, 46, 47, 49, 50, 51, 52, 53, 54, 55, 58, 59, 60, 62, 63, 64, 65, 67, 68, 69, 70, 71, 72, 73, 75, 77, 79, 80, 81, 82, 83, 86, 88, 89, 90, 92, 93, 94, 96, 98, 99, 100, 103, 104, 106, 107, 108, 110, 111, 113, 114, 115, 116, 118, 119, 121, 122, 124, 125, 126, 127, 128, 129, 130, 131, 132, 133, 135, 136, 139, 140, 143, 145, 146, 147, 149, 150, 155, 157, 160, 161, 165, 167, 171, 173, 174, 175, 177, 178, 181, 182, 186, 190, 193, 194, 199, 204, 205, 206, 208, 211, 218, 225, 232, 236, 237, 238, 239, 242, 243, 244, 246, 247, 252, 260, 265, 266, 851, 852, 853, 854, 855, 856 (hereinafter, this group is referred to as “cancer type-specific polynucleotide group 1”), with any of the polynucleotides consisting of nucleotide sequences represented by the other SEQ ID NOs.

The combination of polynucleotides with cancer type specificity capable of discriminating a breast cancer patient not only from a healthy subject but also from other cancer patients is more preferably a combination of multiple polynucleotides, any of which are selected from the cancer type-specific polynucleotide group 1 described above.

The combination of polynucleotides with cancer type specificity capable of discriminating a breast cancer patient not only from a healthy subject but also from other cancer patients is more preferably a combination comprising at least one polynucleotide selected from, particularly, the group consisting of polynucleotides of SEQ ID NOs: 1, 2, 3, 4, 5, 6, 7, 8, 9, 11, 12, 13, 14, 15, 16, 17, 20, 21, 22, 23, 24, 25, 26, 27, 29, 30, 31, 32, 33, 34, 35, 36, 37, 38, 39, 41, 43, 45, 46, 47, 49, 50, 51, 52, 54, 55, 58, 59, 60, 62, 63, 64, 65, 67, 68, 69, 71, 72, 73, 75, 77, 79, 80, 82, 83, 86, 88, 92, 93, 96, 99, 103, 104, 106, 110, 111, 114, 116, 118, 119, 122, 124, 125, 127, 130, 132, 133, 135, 139, 143, 145, 147, 149, 157, 160, 173, 177, 181, 182, 186, 211, 218, 232, 236, 237, 238, 239, 242, 243, 246, 247, 260, 266, 851, 852, 853, 854 (hereinafter, this group is referred to as “cancer type-specific polynucleotide group 2”), among the combinations of multiple polynucleotides selected from the cancer type-specific polynucleotide group 1 described above.

The number of the polynucleotides with cancer type specificity in the combination described above can be 1, 2, 3, 4, 5, 6, 7, 8, 9, 10 or more and is preferably 2 or more for the combination.

Non-limiting examples of the combination of two polynucleotides that consist of one polynucleotide that consists of a nucleotide sequence selected from the cancer type-specific polynucleotide group 2 or a complementary sequence thereof and one polynucleotide that consists of a nucleotide sequence selected from the cancer type-specific polynucleotide group 1 or a complementary sequence thereof are listed below.

(1-1) a combination of SEQ ID NOs: 2 and 1 (markers: hsa-miR-4730 and hsa-miR-4783-3p);
(1-2) a combination of SEQ ID NOs: 2 and 237 (markers: hsa-miR-4730 and hsa-miR-602);
(1-3) a combination of SEQ ID NOs: 2 and 4 (markers: hsa-miR-4730 and hsa-miR-4634);
(1-4) a combination of SEQ ID NOs: 2 and 3 (markers: hsa-miR-4730 and hsa-miR-1307-3p);
(1-5) a combination of SEQ ID NOs: 2 and 51 (markers: hsa-miR-4730 and hsa-miR-3940-5p);
(2-1) a combination of SEQ ID NOs: 1 and 237 (markers: hsa-miR-4783-3p and hsa-miR-602);
(2-2) a combination of SEQ ID NOs: 1 and 4 (markers: hsa-miR-4783-3p and hsa-miR-4634);
(2-3) a combination of SEQ ID NOs: 1 and 3 (markers: hsa-miR-4783-3p and hsa-miR-1307-3p);
(2-4) a combination of SEQ ID NOs: 1 and 51 (markers: hsa-miR-4783-3p and hsa-miR-3940-5p);
(2-5) a combination of SEQ ID NOs: 1 and 6 (markers: hsa-miR-4783-3p and hsa-miR-4532);
(3-1) a combination of SEQ ID NOs: 4 and 237 (markers: hsa-miR-4634 and hsa-miR-602);
(3-2) a combination of SEQ ID NOs: 3 and 237 (markers: hsa-miR-1307-3p and hsa-miR-602);
(3-3) a combination of SEQ ID NOs: 51 and 237 (markers: hsa-miR-3940-5p and hsa-miR-602);
(3-4) a combination of SEQ ID NOs: 237 and 6 (markers: hsa-miR-602 and hsa-miR-4532);
(3-5) a combination of SEQ ID NOs: 237 and 12 (markers: hsa-miR-602 and hsa-miR-3184-5p);
(4-1) a combination of SEQ ID NOs: 3 and 4 (markers: hsa-miR-1307-3p and hsa-miR-4634);
(4-2) a combination of SEQ ID NOs: 4 and 51 (markers: hsa-miR-4634 and hsa-miR-3940-5p);
(4-3) a combination of SEQ ID NOs: 4 and 6 (markers: hsa-miR-4634 and hsa-miR-4532);
(4-4) a combination of SEQ ID NOs: 4 and 12 (markers: hsa-miR-4634 and hsa-miR-3184-5p);
(4-5) a combination of SEQ ID NOs: 4 and 15 (markers: hsa-miR-4634 and hsa-miR-4674);
(5-1) a combination of SEQ ID NOs: 3 and 51 (markers: hsa-miR-1307-3p and hsa-miR-3940-5p);
(5-2) a combination of SEQ ID NOs: 3 and 6 (markers: hsa-miR-1307-3p and hsa-miR-4532);
(5-3) a combination of SEQ ID NOs: 3 and 12 (markers: hsa-miR-1307-3p and hsa-miR-3184-5p);
(5-4) a combination of SEQ ID NOs: 3 and 15 (markers: hsa-miR-1307-3p and hsa-miR-4674);
(5-5) a combination of SEQ ID NOs: 3 and 8 (markers: hsa-miR-1307-3p and hsa-miR-3178);
(6-1) a combination of SEQ ID NOs: 51 and 6 (markers: hsa-miR-3940-5p and hsa-miR-4532);
(6-2) a combination of SEQ ID NOs: 51 and 12 (markers: hsa-miR-3940-5p and hsa-miR-3184-5p);
(6-3) a combination of SEQ ID NOs: 51 and 15 (markers: hsa-miR-3940-5p and hsa-miR-4674);
(6-4) a combination of SEQ ID NOs: 51 and 8 (markers: hsa-miR-3940-5p and hsa-miR-3178);
(6-5) a combination of SEQ ID NOs: 51 and 34 (markers: hsa-miR-3940-5p and hsa-miR-6805-5p);
(7-1) a combination of SEQ ID NOs: 2 and 6 (markers: hsa-miR-4730 and hsa-miR-4532);
(7-2) a combination of SEQ ID NOs: 12 and 6 (markers: hsa-miR-3184-5p and hsa-miR-4532);
(7-3) a combination of SEQ ID NOs: 15 and 6 (markers: hsa-miR-4674 and hsa-miR-4532);
(7-4) a combination of SEQ ID NOs: 8 and 6 (markers: hsa-miR-3178 and hsa-miR-4532);
(7-5) a combination of SEQ ID NOs: 6 and 34 (markers: hsa-miR-4532 and hsa-miR-6805-5p);
(8-1) a combination of SEQ ID NOs: 2 and 12 (markers: hsa-miR-4730 and hsa-miR-3184-5p);
(8-2) a combination of SEQ ID NOs: 1 and 12 (markers: hsa-miR-4783-3p and hsa-miR-3184-5p);
(8-3) a combination of SEQ ID NOs: 12 and 15 (markers: hsa-miR-3184-5p and hsa-miR-4674);
(8-4) a combination of SEQ ID NOs: 8 and 12 (markers: hsa-miR-3178 and hsa-miR-3184-5p);
(8-5) a combination of SEQ ID NOs: 12 and 34 (markers: hsa-miR-3184-5p and hsa-miR-6805-5p);
(9-1) a combination of SEQ ID NOs: 2 and 15 (markers: hsa-miR-4730 and hsa-miR-4674);
(9-2) a combination of SEQ ID NOs: 1 and 15 (markers: hsa-miR-4783-3p and hsa-miR-4674);
(9-3) a combination of SEQ ID NOs: 237 and 15 (markers: hsa-miR-602 and hsa-miR-4674);
(9-4) a combination of SEQ ID NOs: 8 and 15 (markers: hsa-miR-3178 and hsa-miR-4674);
(9-5) a combination of SEQ ID NOs: 15 and 34 (markers: hsa-miR-4674 and hsa-miR-6805-5p);
(10-1) a combination of SEQ ID NOs: 2 and 8 (markers: hsa-miR-4730 and hsa-miR-3178);
(10-2) a combination of SEQ ID NOs: 1 and 8 (markers: hsa-miR-4783-3p and hsa-miR-3178);
(10-3) a combination of SEQ ID NOs: 237 and 8 (markers: hsa-miR-602 and hsa-miR-3178);
(10-4) a combination of SEQ ID NOs: 4 and 8 (markers: hsa-miR-4634 and hsa-miR-3178);
(10-5) a combination of SEQ ID NOs:8 and 34 (markers: hsa-miR-3178 and hsa-miR-6805-5p);
(11-1) a combination of SEQ ID NOs: 2 and 34 (markers: hsa-miR-4730 and hsa-miR-6805-5p);
(11-2) a combination of SEQ ID NOs: 1 and 34 (markers: hsa-miR-4783-3p and hsa-miR-6805-5p);
(11-3) a combination of SEQ ID NOs: 237 and 34 (markers: hsa-miR-602 and hsa-miR-6805-5p);
(11-4) a combination of SEQ ID NOs: 4 and 34 (markers: hsa-miR-4634 and hsa-miR-6805-5p);
(11-5) a combination of SEQ ID NOs: 3 and 34 (markers: hsa-miR-1307-3p and hsa-miR-6805-5p);
(12-1) a combination of SEQ ID NOs: 2 and 9 (markers: hsa-miR-4730 and hsa-miR-6729-5p);
(12-2) a combination of SEQ ID NOs: 1 and 9 (markers: hsa-miR-4783-3p and hsa-miR-6729-5p);
(12-3) a combination of SEQ ID NOs: 9 and 237 (markers: hsa-miR-6729-5p and hsa-miR-602);
(12-4) a combination of SEQ ID NOs: 4 and 9 (markers: hsa-miR-4634 and hsa-miR-6729-5p);
(12-5) a combination of SEQ ID NOs: 3 and 9 (markers: hsa-miR-1307-3p and hsa-miR-6729-5p);
(13-1) a combination of SEQ ID NOs: 2 and 143 (markers: hsa-miR-4730 and hsa-miR-1228-5p);
(13-2) a combination of SEQ ID NOs: 1 and 143 (markers: hsa-miR-4783-3p and hsa-miR-1228-5p);
(13-3) a combination of SEQ ID NOs: 237 and 143 (markers: hsa-miR-602 and hsa-miR-1228-5p);
(13-4) a combination of SEQ ID NOs: 4 and 143 (markers: hsa-miR-4634 and hsa-miR-1228-5p);
(13-5) a combination of SEQ ID NOs: 3 and 143 (markers: hsa-miR-1307-3p and hsa-miR-1228-5p);
(14-1) a combination of SEQ ID NOs: 2 and 13 (markers: hsa-miR-4730 and hsa-miR-6727-5p);
(14-2) a combination of SEQ ID NOs: 1 and 13 (markers: hsa-miR-4783-3p and hsa-miR-6727-5p);
(14-3) a combination of SEQ ID NOs: 237 and 13 (markers: hsa-miR-602 and hsa-miR-6727-5p);
(14-4) a combination of SEQ ID NOs: 4 and 13 (markers: hsa-miR-4634 and hsa-miR-6727-5p);
(14-5) a combination of SEQ ID NOs: 3 and 13 (markers: hsa-miR-1307-3p and hsa-miR-6727-5p);
(15-1) a combination of SEQ ID NOs: 2 and 125 (markers: hsa-miR-4730 and hsa-miR-6781-5p);
(15-2) a combination of SEQ ID NOs: 1 and 125 (markers: hsa-miR-4783-3p and hsa-miR-6781-5p);
(15-3) a combination of SEQ ID NOs: 237 and 125 (markers: hsa-miR-602 and hsa-miR-6781-5p);
(15-4) a combination of SEQ ID NOs: 4 and 125 (markers: hsa-miR-4634 and hsa-miR-6781-5p);
(15-5) a combination of SEQ ID NOs: 3 and 125 (markers: hsa-miR-1307-3p and hsa-miR-6781-5p);
(16-1) a combination of SEQ ID NOs: 2 and 236 (markers: hsa-miR-4730 and hsa-miR-760);
(16-2) a combination of SEQ ID NOs: 1 and 236 (markers: hsa-miR-4783-3p and hsa-miR-760);
(16-3) a combination of SEQ ID NOs: 237 and 236 (markers: hsa-miR-602 and hsa-miR-760);
(16-4) a combination of SEQ ID NOs: 4 and 236 (markers: hsa-miR-4634 and hsa-miR-760);
(16-5) a combination of SEQ ID NOs: 3 and 236 (markers: hsa-miR-1307-3p and hsa-miR-760);
(17-1) a combination of SEQ ID NOs: 2 and 46 (markers: hsa-miR-4730 and hsa-miR-1231);
(17-2) a combination of SEQ ID NOs: 1 and 46 (markers: hsa-miR-4783-3p and hsa-miR-1231);
(17-3) a combination of SEQ ID NOs: 237 and 46 (markers: hsa-miR-602 and hsa-miR-1231);
(17-4) a combination of SEQ ID NOs: 4 and 46 (markers: hsa-miR-4634 and hsa-miR-1231);
(17-5) a combination of SEQ ID NOs: 3 and 46 (markers: hsa-miR-1307-3p and hsa-miR-1231);
(18-1) a combination of SEQ ID NOs: 2 and 32 (markers: hsa-miR-4730 and hsa-miR-6836-3p);
(18-2) a combination of SEQ ID NOs: 1 and 32 (markers: hsa-miR-4783-3p and hsa-miR-6836-3p);
(18-3) a combination of SEQ ID NOs: 237 and 32 (markers: hsa-miR-602 and hsa-miR-6836-3p);
(18-4) a combination of SEQ ID NOs: 4 and 32 (markers: hsa-miR-4634 and hsa-miR-6836-3p);
(18-5) a combination of SEQ ID NOs: 3 and 32 (markers: hsa-miR-1307-3p and hsa-miR-6836-3p);
(19-1) a combination of SEQ ID NOs: 2 and 62 (markers: hsa-miR-4730 and hsa-miR-4492);
(19-2) a combination of SEQ ID NOs: 1 and 62 (markers: hsa-miR-4783-3p and hsa-miR-4492);
(19-3) a combination of SEQ ID NOs: 237 and 62 (markers: hsa-miR-602 and hsa-miR-4492);
(19-4) a combination of SEQ ID NOs: 4 and 62 (markers: hsa-miR-4634 and hsa-miR-4492);
(19-5) a combination of SEQ ID NOs: 3 and 62 (markers: hsa-miR-1307-3p and hsa-miR-4492);
(20-1) a combination of SEQ ID NOs: 2 and 88 (markers: hsa-miR-4730 and hsa-miR-4484);
(20-2) a combination of SEQ ID NOs: 1 and 88 (markers: hsa-miR-4783-3p and hsa-miR-4484);
(20-3) a combination of SEQ ID NOs: 237 and 88 (markers: hsa-miR-602 and hsa-miR-4484);
(20-4) a combination of SEQ ID NOs: 4 and 88 (markers: hsa-miR-4634 and hsa-miR-4484);
(20-5) a combination of SEQ ID NOs: 3 and 88 (markers: hsa-miR-1307-3p and hsa-miR-4484);
(21-1) a combination of SEQ ID NOs: 2 and 52 (markers: hsa-miR-4730 and hsa-miR-4741);
(21-2) a combination of SEQ ID NOs: 1 and 52 (markers: hsa-miR-4783-3p and hsa-miR-4741);
(21-3) a combination of SEQ ID NOs: 237 and 52 (markers: hsa-miR-602 and hsa-miR-4741);
(21-4) a combination of SEQ ID NOs: 4 and 52 (markers: hsa-miR-4634 and hsa-miR-4741);
(21-5) a combination of SEQ ID NOs: 3 and 52 (markers: hsa-miR-1307-3p and hsa-miR-4741);
(22-1) a combination of SEQ ID NOs: 2 and 7 (markers: hsa-miR-4730 and hsa-miR-7704);
(22-2) a combination of SEQ ID NOs: 1 and 7 (markers: hsa-miR-4783-3p and hsa-miR-7704);
(22-3) a combination of SEQ ID NOs: 237 and 7 (markers: hsa-miR-602 and hsa-miR-7704);
(22-4) a combination of SEQ ID NOs: 4 and 7 (markers: hsa-miR-4634 and hsa-miR-7704);
(22-5) a combination of SEQ ID NOs: 3 and 7 (markers: hsa-miR-1307-3p and hsa-miR-7704);
(23-1) a combination of SEQ ID NOs: 2 and 26 (markers: hsa-miR-4730 and hsa-miR-6875-5p);
(23-2) a combination of SEQ ID NOs: 1 and 26 (markers: hsa-miR-4783-3p and hsa-miR-6875-5p);
(23-3) a combination of SEQ ID NOs: 237 and 26 (markers: hsa-miR-602 and hsa-miR-6875-5p);
(23-4) a combination of SEQ ID NOs: 4 and 26 (markers: hsa-miR-4634 and hsa-miR-6875-5p);
(23-5) a combination of SEQ ID NOs: 3 and 26 (markers: hsa-miR-1307-3p and hsa-miR-6875-5p);
(24-1) a combination of SEQ ID NOs: 2 and 25 (markers: hsa-miR-4730 and hsa-miR-8069);
(24-2) a combination of SEQ ID NOs: 1 and 25 (markers: hsa-miR-4783-3p and hsa-miR-8069);
(24-3) a combination of SEQ ID NOs: 237 and 25 (markers: hsa-miR-602 and hsa-miR-8069);
(24-4) a combination of SEQ ID NOs: 4 and 25 (markers: hsa-miR-4634 and hsa-miR-8069);
(24-5) a combination of SEQ ID NOs: 3 and 25 (markers: hsa-miR-1307-3p and hsa-miR-8069);
(25-1) a combination of SEQ ID NOs: 2 and 54 (markers: hsa-miR-4730 and hsa-miR-6869-5p);
(25-2) a combination of SEQ ID NOs: 1 and 54 (markers: hsa-miR-4783-3p and hsa-miR-6869-5p);
(25-3) a combination of SEQ ID NOs: 237 and 54 (markers: hsa-miR-602 and hsa-miR-6869-5p);
(25-4) a combination of SEQ ID NOs: 4 and 54 (markers: hsa-miR-4634 and hsa-miR-6869-5p);
(25-5) a combination of SEQ ID NOs: 3 and 54 (markers: hsa-miR-1307-3p and hsa-miR-6869-5p);
(26-1) a combination of SEQ ID NOs: 2 and 92 (markers: hsa-miR-4730 and hsa-miR-4725-3p);
(26-2) a combination of SEQ ID NOs: 1 and 92 (markers: hsa-miR-4783-3p and hsa-miR-4725-3p);
(26-3) a combination of SEQ ID NOs: 237 and 92 (markers: hsa-miR-602 and hsa-miR-4725-3p);
(26-4) a combination of SEQ ID NOs: 4 and 92 (markers: hsa-miR-4634 and hsa-miR-4725-3p);
(26-5) a combination of SEQ ID NOs: 3 and 92 (markers: hsa-miR-1307-3p and hsa-miR-4725-3p);
(27-1) a combination of SEQ ID NOs: 2 and 14 (markers: hsa-miR-4730 and hsa-miR-6088);
(27-2) a combination of SEQ ID NOs: 1 and 14 (markers: hsa-miR-4783-3p and hsa-miR-6088);
(27-3) a combination of SEQ ID NOs: 237 and 14 (markers: hsa-miR-602 and hsa-miR-6088);
(27-4) a combination of SEQ ID NOs: 4 and 14 (markers: hsa-miR-4634 and hsa-miR-6088);
(27-5) a combination of SEQ ID NOs: 3 and 14 (markers: hsa-miR-1307-3p and hsa-miR-6088);
(28-1) a combination of SEQ ID NOs: 2 and 242 (markers: hsa-miR-4730 and hsa-miR-135a-3p);
(28-2) a combination of SEQ ID NOs: 1 and 242 (markers: hsa-miR-4783-3p and hsa-miR-135a-3p);
(28-3) a combination of SEQ ID NOs: 237 and 242 (markers: hsa-miR-602 and hsa-miR-135a-3p);
(28-4) a combination of SEQ ID NOs: 4 and 242 (markers: hsa-miR-4634 and hsa-miR-135a-3p);
(28-5) a combination of SEQ ID NOs: 3 and 242 (markers: hsa-miR-1307-3p and hsa-miR-135a-3p);
(29-1) a combination of SEQ ID NOs: 2 and 47 (markers: hsa-miR-4730 and hsa-miR-1908-3p);
(29-2) a combination of SEQ ID NOs: 1 and 47 (markers: hsa-miR-4783-3p and hsa-miR-1908-3p);
(29-3) a combination of SEQ ID NOs: 237 and 47 (markers: hsa-miR-602 and hsa-miR-1908-3p);
(29-4) a combination of SEQ ID NOs: 4 and 47 (markers: hsa-miR-4634 and hsa-miR-1908-3p);
(29-5) a combination of SEQ ID NOs: 3 and 47 (markers: hsa-miR-1307-3p and hsa-miR-1908-3p);
(30-1) a combination of SEQ ID NOs: 2 and 45 (markers: hsa-miR-4730 and hsa-miR-6771-5p);
(30-2) a combination of SEQ ID NOs: 1 and 45 (markers: hsa-miR-4783-3p and hsa-miR-6771-5p);
(30-3) a combination of SEQ ID NOs: 237 and 45 (markers: hsa-miR-602 and hsa-miR-6771-5p);
(30-4) a combination of SEQ ID NOs: 4 and 45 (markers: hsa-miR-4634 and hsa-miR-6771-5p);
(30-5) a combination of SEQ ID NOs: 3 and 45 (markers: hsa-miR-1307-3p and hsa-miR-6771-5p);
(31-1) a combination of SEQ ID NOs: 2 and 39 (markers: hsa-miR-4730 and hsa-miR-6850-5p);
(31-2) a combination of SEQ ID NOs: 1 and 39 (markers: hsa-miR-4783-3p and hsa-miR-6850-5p);
(31-3) a combination of SEQ ID NOs: 237 and 39 (markers: hsa-miR-602 and hsa-miR-6850-5p);
(31-4) a combination of SEQ ID NOs: 4 and 39 (markers: hsa-miR-4634 and hsa-miR-6850-5p);
(31-5) a combination of SEQ ID NOs: 3 and 39 (markers: hsa-miR-1307-3p and hsa-miR-6850-5p);
(32-1) a combination of SEQ ID NOs: 2 and 21 (markers: hsa-miR-4730 and hsa-miR-885-3p);
(32-2) a combination of SEQ ID NOs: 1 and 21 (markers: hsa-miR-4783-3p and hsa-miR-885-3p);
(32-3) a combination of SEQ ID NOs: 237 and 21 (markers: hsa-miR-602 and hsa-miR-885-3p);
(32-4) a combination of SEQ ID NOs: 4 and 21 (markers: hsa-miR-4634 and hsa-miR-885-3p);
(32-5) a combination of SEQ ID NOs: 3 and 21 (markers: hsa-miR-1307-3p and hsa-miR-885-3p);
(33-1) a combination of SEQ ID NOs: 2 and 17 (markers: hsa-miR-4730 and hsa-miR-4787-5p);
(33-2) a combination of SEQ ID NOs: 1 and 17 (markers: hsa-miR-4783-3p and hsa-miR-4787-5p);
(33-3) a combination of SEQ ID NOs: 237 and 17 (markers: hsa-miR-602 and hsa-miR-4787-5p);
(33-4) a combination of SEQ ID NOs: 4 and 17 (markers: hsa-miR-4634 and hsa-miR-4787-5p);
(33-5) a combination of SEQ ID NOs: 3 and 17 (markers: hsa-miR-1307-3p and hsa-miR-4787-5p);
(34-1) a combination of SEQ ID NOs: 2 and 83 (markers: hsa-miR-4730 and hsa-miR-1909-3p);
(34-2) a combination of SEQ ID NOs: 1 and 83 (markers: hsa-miR-4783-3p and hsa-miR-1909-3p);
(34-3) a combination of SEQ ID NOs: 237 and 83 (markers: hsa-miR-602 and hsa-miR-1909-3p);
(34-4) a combination of SEQ ID NOs: 4 and 83 (markers: hsa-miR-4634 and hsa-miR-1909-3p);
(34-5) a combination of SEQ ID NOs: 3 and 83 (markers: hsa-miR-1307-3p and hsa-miR-1909-3p);
(35-1) a combination of SEQ ID NOs: 2 and 149 (markers: hsa-miR-4730 and hsa-miR-638);
(35-2) a combination of SEQ ID NOs: 1 and 149 (markers: hsa-miR-4783-3p and hsa-miR-638);
(35-3) a combination of SEQ ID NOs: 237 and 149 (markers: hsa-miR-602 and hsa-miR-638);
(35-4) a combination of SEQ ID NOs: 4 and 149 (markers: hsa-miR-4634 and hsa-miR-638);
(35-5) a combination of SEQ ID NOs: 3 and 149 (markers: hsa-miR-1307-3p and hsa-miR-638);
(36-1) a combination of SEQ ID NOs: 2 and 246 (markers: hsa-miR-4730 and hsa-miR-1915-3p);
(36-2) a combination of SEQ ID NOs: 1 and 246 (markers: hsa-miR-4783-3p and hsa-miR-1915-3p);
(36-3) a combination of SEQ ID NOs: 237 and 246 (markers: hsa-miR-602 and hsa-miR-1915-3p);
(36-4) a combination of SEQ ID NOs: 4 and 246 (markers: hsa-miR-4634 and hsa-miR-1915-3p);
(36-5) a combination of SEQ ID NOs: 3 and 246 (markers: hsa-miR-1307-3p and hsa-miR-1915-3p);
(37-1) a combination of SEQ ID NOs: 2 and 22 (markers: hsa-miR-4730 and hsa-miR-6802-5p);
(37-2) a combination of SEQ ID NOs: 1 and 22 (markers: hsa-miR-4783-3p and hsa-miR-6802-5p);
(37-3) a combination of SEQ ID NOs: 237 and 22 (markers: hsa-miR-602 and hsa-miR-6802-5p);
(37-4) a combination of SEQ ID NOs: 4 and 22 (markers: hsa-miR-4634 and hsa-miR-6802-5p);
(37-5) a combination of SEQ ID NOs: 3 and 22 (markers: hsa-miR-1307-3p and hsa-miR-6802-5p);
(38-1) a combination of SEQ ID NOs: 2 and 55 (markers: hsa-miR-4730 and hsa-miR-5090);
(38-2) a combination of SEQ ID NOs: 1 and 55 (markers: hsa-miR-4783-3p and hsa-miR-5090);
(38-3) a combination of SEQ ID NOs: 237 and 55 (markers: hsa-miR-602 and hsa-miR-5090);
(38-4) a combination of SEQ ID NOs: 4 and 55 (markers: hsa-miR-4634 and hsa-miR-5090);
(38-5) a combination of SEQ ID NOs: 3 and 55 (markers: hsa-miR-1307-3p and hsa-miR-5090);
(39-1) a combination of SEQ ID NOs: 2 and 182 (markers: hsa-miR-4730 and hsa-miR-3196);
(39-2) a combination of SEQ ID NOs: 1 and 182 (markers: hsa-miR-4783-3p and hsa-miR-3196);
(39-3) a combination of SEQ ID NOs: 237 and 182 (markers: hsa-miR-602 and hsa-miR-3196);
(39-4) a combination of SEQ ID NOs: 4 and 182 (markers: hsa-miR-4634 and hsa-miR-3196);
(39-5) a combination of SEQ ID NOs: 3 and 182 (markers: hsa-miR-1307-3p and hsa-miR-3196);
(40-1) a combination of SEQ ID NOs: 2 and 73 (markers: hsa-miR-4730 and hsa-miR-4707-3p);
(40-2) a combination of SEQ ID NOs: 1 and 73 (markers: hsa-miR-4783-3p and hsa-miR-4707-3p);
(40-3) a combination of SEQ ID NOs: 237 and 73 (markers: hsa-miR-602 and hsa-miR-4707-3p);
(40-4) a combination of SEQ ID NOs: 4 and 73 (markers: hsa-miR-4634 and hsa-miR-4707-3p);
(40-5) a combination of SEQ ID NOs: 3 and 73 (markers: hsa-miR-1307-3p and hsa-miR-4707-3p);
(41-1) a combination of SEQ ID NOs: 2 and 77 (markers: hsa-miR-4730 and hsa-miR-4651);
(41-2) a combination of SEQ ID NOs: 1 and 77 (markers: hsa-miR-4783-3p and hsa-miR-4651);
(41-3) a combination of SEQ ID NOs: 237 and 77 (markers: hsa-miR-602 and hsa-miR-4651);
(41-4) a combination of SEQ ID NOs: 4 and 77 (markers: hsa-miR-4634 and hsa-miR-4651);
(41-5) a combination of SEQ ID NOs: 3 and 77 (markers: hsa-miR-1307-3p and hsa-miR-4651);
(42-1) a combination of SEQ ID NOs: 2 and 24 (markers: hsa-miR-4730 and hsa-miR-6787-5p);
(42-2) a combination of SEQ ID NOs: 1 and 24 (markers: hsa-miR-4783-3p and hsa-miR-6787-5p);
(42-3) a combination of SEQ ID NOs: 237 and 24 (markers: hsa-miR-602 and hsa-miR-6787-5p);
(42-4) a combination of SEQ ID NOs: 4 and 24 (markers: hsa-miR-4634 and hsa-miR-6787-5p);
(42-5) a combination of SEQ ID NOs: 3 and 24 (markers: hsa-miR-1307-3p and hsa-miR-6787-5p);
(43-1) a combination of SEQ ID NOs: 2 and 103 (markers: hsa-miR-4730 and hsa-miR-3180-3p);
(43-2) a combination of SEQ ID NOs: 1 and 103 (markers: hsa-miR-4783-3p and hsa-miR-3180-3p);
(43-3) a combination of SEQ ID NOs: 237 and 103 (markers: hsa-miR-602 and hsa-miR-3180-3p);
(43-4) a combination of SEQ ID NOs: 4 and 103 (markers: hsa-miR-4634 and hsa-miR-3180-3p);
(43-5) a combination of SEQ ID NOs: 3 and 103 (markers: hsa-miR-1307-3p and hsa-miR-3180-3p);
(44-1) a combination of SEQ ID NOs: 2 and 49 (markers: hsa-miR-4730 and hsa-miR-3937);
(44-2) a combination of SEQ ID NOs: 1 and 49 (markers: hsa-miR-4783-3p and hsa-miR-3937);
(44-3) a combination of SEQ ID NOs: 237 and 49 (markers: hsa-miR-602 and hsa-miR-3937);
(44-4) a combination of SEQ ID NOs: 4 and 49 (markers: hsa-miR-4634 and hsa-miR-3937);
(44-5) a combination of SEQ ID NOs: 3 and 49 (markers: hsa-miR-1307-3p and hsa-miR-3937);
(45-1) a combination of SEQ ID NOs: 2 and 239 (markers: hsa-miR-4730 and hsa-miR-92a-2-5p);
(45-2) a combination of SEQ ID NOs: 1 and 239 (markers: hsa-miR-4783-3p and hsa-miR-92a-2-5p);
(45-3) a combination of SEQ ID NOs: 237 and 239 (markers: hsa-miR-602 and hsa-miR-92a-2-5p);
(45-4) a combination of SEQ ID NOs: 4 and 239 (markers: hsa-miR-4634 and hsa-miR-92a-2-5p);
(45-5) a combination of SEQ ID NOs: 3 and 239 (markers: hsa-miR-1307-3p and hsa-miR-92a-2-5p);
(46-1) a combination of SEQ ID NOs: 2 and 23 (markers: hsa-miR-4730 and hsa-miR-328-5p);
(46-2) a combination of SEQ ID NOs: 1 and 23 (markers: hsa-miR-4783-3p and hsa-miR-328-5p);
(46-3) a combination of SEQ ID NOs: 237 and 23 (markers: hsa-miR-602 and hsa-miR-328-5p);
(46-4) a combination of SEQ ID NOs: 4 and 23 (markers: hsa-miR-4634 and hsa-miR-328-5p);
(46-5) a combination of SEQ ID NOs: 3 and 23 (markers: hsa-miR-1307-3p and hsa-miR-328-5p);
(47-1) a combination of SEQ ID NOs: 2 and 58 (markers: hsa-miR-4730 and hsa-miR-128-1-5p);
(47-2) a combination of SEQ ID NOs: 1 and 58 (markers: hsa-miR-4783-3p and hsa-miR-128-1-5p);
(47-3) a combination of SEQ ID NOs: 237 and 58 (markers: hsa-miR-602 and hsa-miR-128-1-5p);
(47-4) a combination of SEQ ID NOs: 4 and 58 (markers: hsa-miR-4634 and hsa-miR-128-1-5p);
(47-5) a combination of SEQ ID NOs: 3 and 58 (markers: hsa-miR-1307-3p and hsa-miR-128-1-5p);
(48-1) a combination of SEQ ID NOs: 2 and 211 (markers: hsa-miR-4730 and hsa-miR-6765-5p);
(48-2) a combination of SEQ ID NOs: 1 and 211 (markers: hsa-miR-4783-3p and hsa-miR-6765-5p);
(48-3) a combination of SEQ ID NOs: 237 and 211 (markers: hsa-miR-602 and hsa-miR-6765-5p);
(48-4) a combination of SEQ ID NOs: 4 and 211 (markers: hsa-miR-4634 and hsa-miR-6765-5p);
(48-5) a combination of SEQ ID NOs: 3 and 211 (markers: hsa-miR-1307-3p and hsa-miR-6765-5p);
(49-1) a combination of SEQ ID NOs: 2 and 147 (markers: hsa-miR-4730 and hsa-miR-6786-5p);
(49-2) a combination of SEQ ID NOs: 1 and 147 (markers: hsa-miR-4783-3p and hsa-miR-6786-5p);
(49-3) a combination of SEQ ID NOs: 237 and 147 (markers: hsa-miR-602 and hsa-miR-6786-5p);
(49-4) a combination of SEQ ID NOs: 4 and 147 (markers: hsa-miR-4634 and hsa-miR-6786-5p);
(49-5) a combination of SEQ ID NOs: 3 and 147 (markers: hsa-miR-1307-3p and hsa-miR-6786-5p);
(50-1) a combination of SEQ ID NOs: 2 and 65 (markers: hsa-miR-4730 and hsa-miR-4525);
(50-2) a combination of SEQ ID NOs: 1 and 65 (markers: hsa-miR-4783-3p and hsa-miR-4525);
(50-3) a combination of SEQ ID NOs: 237 and 65 (markers: hsa-miR-602 and hsa-miR-4525);
(50-4) a combination of SEQ ID NOs: 4 and 65 (markers: hsa-miR-4634 and hsa-miR-4525);
(50-5) a combination of SEQ ID NOs: 3 and 65 (markers: hsa-miR-1307-3p and hsa-miR-4525);
(51-1) a combination of SEQ ID NOs: 2 and 31 (markers: hsa-miR-4730 and hsa-miR-3665);
(51-2) a combination of SEQ ID NOs: 1 and 31 (markers: hsa-miR-4783-3p and hsa-miR-3665);
(51-3) a combination of SEQ ID NOs: 237 and 31 (markers: hsa-miR-602 and hsa-miR-3665);
(51-4) a combination of SEQ ID NOs: 4 and 31 (markers: hsa-miR-4634 and hsa-miR-3665);
(51-5) a combination of SEQ ID NOs: 3 and 31 (markers: hsa-miR-1307-3p and hsa-miR-3665);
(52-1) a combination of SEQ ID NOs: 2 and 72 (markers: hsa-miR-4730 and hsa-miR-663b);
(52-2) a combination of SEQ ID NOs: 1 and 72 (markers: hsa-miR-4783-3p and hsa-miR-663b);
(52-3) a combination of SEQ ID NOs: 237 and 72 (markers: hsa-miR-602 and hsa-miR-663b);
(52-4) a combination of SEQ ID NOs: 4 and 72 (markers: hsa-miR-4634 and hsa-miR-663b);
(52-5) a combination of SEQ ID NOs: 3 and 72 (markers: hsa-miR-1307-3p and hsa-miR-663b);
(53-1) a combination of SEQ ID NOs: 2 and 63 (markers: hsa-miR-4730 and hsa-miR-6785-5p);
(53-2) a combination of SEQ ID NOs: 1 and 63 (markers: hsa-miR-4783-3p and hsa-miR-6785-5p);
(53-3) a combination of SEQ ID NOs: 237 and 63 (markers: hsa-miR-602 and hsa-miR-6785-5p);
(53-4) a combination of SEQ ID NOs: 4 and 63 (markers: hsa-miR-4634 and hsa-miR-6785-5p);
(53-5) a combination of SEQ ID NOs: 3 and 63 (markers: hsa-miR-1307-3p and hsa-miR-6785-5p);
(54-1) a combination of SEQ ID NOs: 2 and 80 (markers: hsa-miR-4730 and hsa-miR-4758-5p);
(54-2) a combination of SEQ ID NOs: 1 and 80 (markers: hsa-miR-4783-3p and hsa-miR-4758-5p);
(54-3) a combination of SEQ ID NOs: 237 and 80 (markers: hsa-miR-602 and hsa-miR-4758-5p);
(54-4) a combination of SEQ ID NOs: 4 and 80 (markers: hsa-miR-4634 and hsa-miR-4758-5p);
(54-5) a combination of SEQ ID NOs: 3 and 80 (markers: hsa-miR-1307-3p and hsa-miR-4758-5p);
(55-1) a combination of SEQ ID NOs: 2 and 37 (markers: hsa-miR-4730 and hsa-miR-197-5p);
(55-2) a combination of SEQ ID NOs: 1 and 37 (markers: hsa-miR-4783-3p and hsa-miR-197-5p);
(55-3) a combination of SEQ ID NOs: 237 and 37 (markers: hsa-miR-602 and hsa-miR-197-5p);
(55-4) a combination of SEQ ID NOs: 4 and 37 (markers: hsa-miR-4634 and hsa-miR-197-5p);
(55-5) a combination of SEQ ID NOs: 3 and 37 (markers: hsa-miR-1307-3p and hsa-miR-197-5p);
(56-1) a combination of SEQ ID NOs: 2 and 67 (markers: hsa-miR-4730 and hsa-miR-3180);
(56-2) a combination of SEQ ID NOs: 1 and 67 (markers: hsa-miR-4783-3p and hsa-miR-3180);
(56-3) a combination of SEQ ID NOs: 237 and 67 (markers: hsa-miR-602 and hsa-miR-3180);
(56-4) a combination of SEQ ID NOs: 4 and 67 (markers: hsa-miR-4634 and hsa-miR-3180);
(56-5) a combination of SEQ ID NOs: 3 and 67 (markers: hsa-miR-1307-3p and hsa-miR-3180);
(57-1) a combination of SEQ ID NOs: 2 and 232 (markers: hsa-miR-4730 and hsa-miR-6125);
(57-2) a combination of SEQ ID NOs: 1 and 232 (markers: hsa-miR-4783-3p and hsa-miR-6125);
(57-3) a combination of SEQ ID NOs: 237 and 232 (markers: hsa-miR-602 and hsa-miR-6125);
(57-4) a combination of SEQ ID NOs: 4 and 232 (markers: hsa-miR-4634 and hsa-miR-6125);
(57-5) a combination of SEQ ID NOs: 3 and 232 (markers: hsa-miR-1307-3p and hsa-miR-6125);
(58-1) a combination of SEQ ID NOs: 2 and 127 (markers: hsa-miR-4730 and hsa-miR-4446-3p);
(58-2) a combination of SEQ ID NOs: 1 and 127 (markers: hsa-miR-4783-3p and hsa-miR-4446-3p);
(58-3) a combination of SEQ ID NOs: 237 and 127 (markers: hsa-miR-602 and hsa-miR-4446-3p);
(58-4) a combination of SEQ ID NOs: 4 and 127 (markers: hsa-miR-4634 and hsa-miR-4446-3p);
(58-5) a combination of SEQ ID NOs: 3 and 127 (markers: hsa-miR-1307-3p and hsa-miR-4446-3p);
(59-1) a combination of SEQ ID NOs: 2 and 145 (markers: hsa-miR-4730 and hsa-miR-1268a);
(59-2) a combination of SEQ ID NOs: 1 and 145 (markers: hsa-miR-4783-3p and hsa-miR-1268a);
(59-3) a combination of SEQ ID NOs: 237 and 145 (markers: hsa-miR-602 and hsa-miR-1268a);
(59-4) a combination of SEQ ID NOs: 4 and 145 (markers: hsa-miR-4634 and hsa-miR-1268a);
(59-5) a combination of SEQ ID NOs: 3 and 145 (markers: hsa-miR-1307-3p and hsa-miR-1268a);
(60-1) a combination of SEQ ID NOs: 2 and 16 (markers: hsa-miR-4730 and hsa-miR-8073);
(60-2) a combination of SEQ ID NOs: 1 and 16 (markers: hsa-miR-4783-3p and hsa-miR-8073);
(60-3) a combination of SEQ ID NOs: 237 and 16 (markers: hsa-miR-602 and hsa-miR-8073);
(60-4) a combination of SEQ ID NOs: 4 and 16 (markers: hsa-miR-4634 and hsa-miR-8073);
(60-5) a combination of SEQ ID NOs: 3 and 16 (markers: hsa-miR-1307-3p and hsa-miR-8073);
(61-1) a combination of SEQ ID NOs: 2 and 11 (markers: hsa-miR-4730 and hsa-miR-4732-5p);
(61-2) a combination of SEQ ID NOs: 1 and 11 (markers: hsa-miR-4783-3p and hsa-miR-4732-5p);
(61-3) a combination of SEQ ID NOs: 237 and 11 (markers: hsa-miR-602 and hsa-miR-4732-5p);
(61-4) a combination of SEQ ID NOs: 4 and 11 (markers: hsa-miR-4634 and hsa-miR-4732-5p);
(61-5) a combination of SEQ ID NOs: 3 and 11 (markers: hsa-miR-1307-3p and hsa-miR-4732-5p);
(62-1) a combination of SEQ ID NOs: 2 and 186 (markers: hsa-miR-4730 and hsa-miR-6789-5p);
(62-2) a combination of SEQ ID NOs: 1 and 186 (markers: hsa-miR-4783-3p and hsa-miR-6789-5p);
(62-3) a combination of SEQ ID NOs: 237 and 186 (markers: hsa-miR-602 and hsa-miR-6789-5p);
(62-4) a combination of SEQ ID NOs: 4 and 186 (markers: hsa-miR-4634 and hsa-miR-6789-5p);
(62-5) a combination of SEQ ID NOs: 3 and 186 (markers: hsa-miR-1307-3p and hsa-miR-6789-5p);
(63-1) a combination of SEQ ID NOs: 2 and 50 (markers: hsa-miR-4730 and hsa-miR-887-3p);
(63-2) a combination of SEQ ID NOs: 1 and 50 (markers: hsa-miR-4783-3p and hsa-miR-887-3p);
(63-3) a combination of SEQ ID NOs: 237 and 50 (markers: hsa-miR-602 and hsa-miR-887-3p);
(63-4) a combination of SEQ ID NOs: 4 and 50 (markers: hsa-miR-4634 and hsa-miR-887-3p);
(63-5) a combination of SEQ ID NOs: 3 and 50 (markers: hsa-miR-1307-3p and hsa-miR-887-3p);
(64-1) a combination of SEQ ID NOs: 2 and 69 (markers: hsa-miR-4730 and hsa-miR-1199-5p);
(64-2) a combination of SEQ ID NOs: 1 and 69 (markers: hsa-miR-4783-3p and hsa-miR-1199-5p);
(64-3) a combination of SEQ ID NOs: 237 and 69 (markers: hsa-miR-602 and hsa-miR-1199-5p);
(64-4) a combination of SEQ ID NOs: 4 and 69 (markers: hsa-miR-4634 and hsa-miR-1199-5p);
(64-5) a combination of SEQ ID NOs: 3 and 69 (markers: hsa-miR-1307-3p and hsa-miR-1199-5p);
(65-1) a combination of SEQ ID NOs: 2 and 33 (markers: hsa-miR-4730 and hsa-miR-6821-5p);
(65-2) a combination of SEQ ID NOs: 1 and 33 (markers: hsa-miR-4783-3p and hsa-miR-6821-5p);
(65-3) a combination of SEQ ID NOs: 237 and 33 (markers: hsa-miR-602 and hsa-miR-6821-5p);
(65-4) a combination of SEQ ID NOs: 4 and 33 (markers: hsa-miR-4634 and hsa-miR-6821-5p);
(65-5) a combination of SEQ ID NOs: 3 and 33 (markers: hsa-miR-1307-3p and hsa-miR-6821-5p);
(66-1) a combination of SEQ ID NOs: 2 and 247 (markers: hsa-miR-4730 and hsa-miR-718);
(66-2) a combination of SEQ ID NOs: 1 and 247 (markers: hsa-miR-4783-3p and hsa-miR-718);
(66-3) a combination of SEQ ID NOs: 237 and 247 (markers: hsa-miR-602 and hsa-miR-718);
(66-4) a combination of SEQ ID NOs: 4 and 247 (markers: hsa-miR-4634 and hsa-miR-718);
(66-5) a combination of SEQ ID NOs: 3 and 247 (markers: hsa-miR-1307-3p and hsa-miR-718);
(67-1) a combination of SEQ ID NOs: 2 and 36 (markers: hsa-miR-4730 and hsa-miR-6726-5p);
(67-2) a combination of SEQ ID NOs: 1 and 36 (markers: hsa-miR-4783-3p and hsa-miR-6726-5p);
(67-3) a combination of SEQ ID NOs: 237 and 36 (markers: hsa-miR-602 and hsa-miR-6726-5p);
(67-4) a combination of SEQ ID NOs: 4 and 36 (markers: hsa-miR-4634 and hsa-miR-6726-5p);
(67-5) a combination of SEQ ID NOs: 3 and 36 (markers: hsa-miR-1307-3p and hsa-miR-6726-5p);
(68-1) a combination of SEQ ID NOs: 2 and 218 (markers: hsa-miR-4730 and hsa-miR-6784-5p);
(68-2) a combination of SEQ ID NOs: 1 and 218 (markers: hsa-miR-4783-3p and hsa-miR-6784-5p);
(68-3) a combination of SEQ ID NOs: 237 and 218 (markers: hsa-miR-602 and hsa-miR-6784-5p);
(68-4) a combination of SEQ ID NOs: 4 and 218 (markers: hsa-miR-4634 and hsa-miR-6784-5p);
(68-5) a combination of SEQ ID NOs: 3 and 218 (markers: hsa-miR-1307-3p and hsa-miR-6784-5p);
(69-1) a combination of SEQ ID NOs: 2 and 43 (markers: hsa-miR-4730 and hsa-miR-4763-3p);
(69-2) a combination of SEQ ID NOs: 1 and 43 (markers: hsa-miR-4783-3p and hsa-miR-4763-3p);
(69-3) a combination of SEQ ID NOs: 237 and 43 (markers: hsa-miR-602 and hsa-miR-4763-3p);
(69-4) a combination of SEQ ID NOs: 4 and 43 (markers: hsa-miR-4634 and hsa-miR-4763-3p);
(69-5) a combination of SEQ ID NOs: 3 and 43 (markers: hsa-miR-1307-3p and hsa-miR-4763-3p);
(70-1) a combination of SEQ ID NOs: 2 and 29 (markers: hsa-miR-4730 and hsa-miR-6757-5p);
(70-2) a combination of SEQ ID NOs: 1 and 29 (markers: hsa-miR-4783-3p and hsa-miR-6757-5p);
(70-3) a combination of SEQ ID NOs: 237 and 29 (markers: hsa-miR-602 and hsa-miR-6757-5p);
(70-4) a combination of SEQ ID NOs: 4 and 29 (markers: hsa-miR-4634 and hsa-miR-6757-5p);
(70-5) a combination of SEQ ID NOs: 3 and 29 (markers: hsa-miR-1307-3p and hsa-miR-6757-5p);
(71-1) a combination of SEQ ID NOs: 2 and 110 (markers: hsa-miR-4730 and hsa-miR-665);
(71-2) a combination of SEQ ID NOs: 1 and 110 (markers: hsa-miR-4783-3p and hsa-miR-665);
(71-3) a combination of SEQ ID NOs: 237 and 110 (markers: hsa-miR-602 and hsa-miR-665);
(71-4) a combination of SEQ ID NOs: 4 and 110 (markers: hsa-miR-4634 and hsa-miR-665);
(71-5) a combination of SEQ ID NOs: 3 and 110 (markers: hsa-miR-1307-3p and hsa-miR-665);
(72-1) a combination of SEQ ID NOs: 2 and 20 (markers: hsa-miR-4730 and hsa-miR-1233-5p);
(72-2) a combination of SEQ ID NOs: 1 and 20 (markers: hsa-miR-4783-3p and hsa-miR-1233-5p);
(72-3) a combination of SEQ ID NOs: 237 and 20 (markers: hsa-miR-602 and hsa-miR-1233-5p);
(72-4) a combination of SEQ ID NOs: 4 and 20 (markers: hsa-miR-4634 and hsa-miR-1233-5p);
(72-5) a combination of SEQ ID NOs: 3 and 20 (markers: hsa-miR-1307-3p and hsa-miR-1233-5p);
(73-1) a combination of SEQ ID NOs: 2 and 157 (markers: hsa-miR-4730 and hsa-miR-1268b);
(73-2) a combination of SEQ ID NOs: 1 and 157 (markers: hsa-miR-4783-3p and hsa-miR-1268b);
(73-3) a combination of SEQ ID NOs: 237 and 157 (markers: hsa-miR-602 and hsa-miR-1268b);
(73-4) a combination of SEQ ID NOs: 4 and 157 (markers: hsa-miR-4634 and hsa-miR-1268b);
(73-5) a combination of SEQ ID NOs: 3 and 157 (markers: hsa-miR-1307-3p and hsa-miR-1268b);
(74-1) a combination of SEQ ID NOs: 2 and 75 (markers: hsa-miR-4730 and hsa-miR-4675);
(74-2) a combination of SEQ ID NOs: 1 and 75 (markers: hsa-miR-4783-3p and hsa-miR-4675);
(74-3) a combination of SEQ ID NOs: 237 and 75 (markers: hsa-miR-602 and hsa-miR-4675);
(74-4) a combination of SEQ ID NOs: 4 and 75 (markers: hsa-miR-4634 and hsa-miR-4675);
(74-5) a combination of SEQ ID NOs: 3 and 75 (markers: hsa-miR-1307-3p and hsa-miR-4675);
(75-1) a combination of SEQ ID NOs: 2 and 82 (markers: hsa-miR-4730 and hsa-miR-3620-5p);
(75-2) a combination of SEQ ID NOs: 1 and 82 (markers: hsa-miR-4783-3p and hsa-miR-3620-5p);
(75-3) a combination of SEQ ID NOs: 237 and 82 (markers: hsa-miR-602 and hsa-miR-3620-5p);
(75-4) a combination of SEQ ID NOs: 4 and 82 (markers: hsa-miR-4634 and hsa-miR-3620-5p);
(75-5) a combination of SEQ ID NOs: 3 and 82 (markers: hsa-miR-1307-3p and hsa-miR-3620-5p);
(76-1) a combination of SEQ ID NOs: 2 and 106 (markers: hsa-miR-4730 and hsa-miR-6800-5p);
(76-2) a combination of SEQ ID NOs: 1 and 106 (markers: hsa-miR-4783-3p and hsa-miR-6800-5p);
(76-3) a combination of SEQ ID NOs: 237 and 106 (markers: hsa-miR-602 and hsa-miR-6800-5p);
(76-4) a combination of SEQ ID NOs: 4 and 106 (markers: hsa-miR-4634 and hsa-miR-6800-5p);
(76-5) a combination of SEQ ID NOs: 3 and 106 (markers: hsa-miR-1307-3p and hsa-miR-6800-5p);
(77-1) a combination of SEQ ID NOs: 2 and 111 (markers: hsa-miR-4730 and hsa-miR-6778-5p);
(77-2) a combination of SEQ ID NOs: 1 and 111 (markers: hsa-miR-4783-3p and hsa-miR-6778-5p);
(77-3) a combination of SEQ ID NOs: 237 and 111 (markers: hsa-miR-602 and hsa-miR-6778-5p);
(77-4) a combination of SEQ ID NOs: 4 and 111 (markers: hsa-miR-4634 and hsa-miR-6778-5p);
(77-5) a combination of SEQ ID NOs: 3 and 111 (markers: hsa-miR-1307-3p and hsa-miR-6778-5p);
(78-1) a combination of SEQ ID NOs: 2 and 96 (markers: hsa-miR-4730 and hsa-miR-8059);
(78-2) a combination of SEQ ID NOs: 1 and 96 (markers: hsa-miR-4783-3p and hsa-miR-8059);
(78-3) a combination of SEQ ID NOs: 237 and 96 (markers: hsa-miR-602 and hsa-miR-8059);
(78-4) a combination of SEQ ID NOs: 4 and 96 (markers: hsa-miR-4634 and hsa-miR-8059);
(78-5) a combination of SEQ ID NOs: 3 and 96 (markers: hsa-miR-1307-3p and hsa-miR-8059);
(79-1) a combination of SEQ ID NOs: 2 and 266 (markers: hsa-miR-4730 and hsa-miR-1908-5p);
(79-2) a combination of SEQ ID NOs: 1 and 266 (markers: hsa-miR-4783-3p and hsa-miR-1908-5p);
(79-3) a combination of SEQ ID NOs: 237 and 266 (markers: hsa-miR-602 and hsa-miR-1908-5p);
(79-4) a combination of SEQ ID NOs: 4 and 266 (markers: hsa-miR-4634 and hsa-miR-1908-5p);
(79-5) a combination of SEQ ID NOs: 3 and 266 (markers: hsa-miR-1307-3p and hsa-miR-1908-5p);
(80-1) a combination of SEQ ID NOs: 2 and 124 (markers: hsa-miR-4730 and hsa-miR-6798-5p);
(80-2) a combination of SEQ ID NOs: 1 and 124 (markers: hsa-miR-4783-3p and hsa-miR-6798-5p);
(80-3) a combination of SEQ ID NOs: 237 and 124 (markers: hsa-miR-602 and hsa-miR-6798-5p);
(80-4) a combination of SEQ ID NOs: 4 and 124 (markers: hsa-miR-4634 and hsa-miR-6798-5p);
(80-5) a combination of SEQ ID NOs: 3 and 124 (markers: hsa-miR-1307-3p and hsa-miR-6798-5p);
(81-1) a combination of SEQ ID NOs: 2 and 68 (markers: hsa-miR-4730 and hsa-miR-6879-5p);
(81-2) a combination of SEQ ID NOs: 1 and 68 (markers: hsa-miR-4783-3p and hsa-miR-6879-5p);
(81-3) a combination of SEQ ID NOs: 237 and 68 (markers: hsa-miR-602 and hsa-miR-6879-5p);
(81-4) a combination of SEQ ID NOs: 4 and 68 (markers: hsa-miR-4634 and hsa-miR-6879-5p);
(81-5) a combination of SEQ ID NOs: 3 and 68 (markers: hsa-miR-1307-3p and hsa-miR-6879-5p);
(82-1) a combination of SEQ ID NOs: 2 and 71 (markers: hsa-miR-4730 and hsa-miR-711);
(82-2) a combination of SEQ ID NOs: 1 and 71 (markers: hsa-miR-4783-3p and hsa-miR-711);
(82-3) a combination of SEQ ID NOs: 237 and 71 (markers: hsa-miR-602 and hsa-miR-711);
(82-4) a combination of SEQ ID NOs: 4 and 71 (markers: hsa-miR-4634 and hsa-miR-711);
(82-5) a combination of SEQ ID NOs: 3 and 71 (markers: hsa-miR-1307-3p and hsa-miR-711);
(83-1) a combination of SEQ ID NOs: 2 and 35 (markers: hsa-miR-4730 and hsa-miR-4728-5p);
(83-2) a combination of SEQ ID NOs: 1 and 35 (markers: hsa-miR-4783-3p and hsa-miR-4728-5p);
(83-3) a combination of SEQ ID NOs: 237 and 35 (markers: hsa-miR-602 and hsa-miR-4728-5p);
(83-4) a combination of SEQ ID NOs: 4 and 35 (markers: hsa-miR-4634 and hsa-miR-4728-5p);
(83-5) a combination of SEQ ID NOs: 3 and 35 (markers: hsa-miR-1307-3p and hsa-miR-4728-5p);
(84-1) a combination of SEQ ID NOs: 2 and 173 (markers: hsa-miR-4730 and hsa-miR-3195);
(84-2) a combination of SEQ ID NOs: 1 and 173 (markers: hsa-miR-4783-3p and hsa-miR-3195);
(84-3) a combination of SEQ ID NOs: 237 and 173 (markers: hsa-miR-602 and hsa-miR-3195);
(84-4) a combination of SEQ ID NOs: 4 and 173 (markers: hsa-miR-4634 and hsa-miR-3195);
(84-5) a combination of SEQ ID NOs: 3 and 173 (markers: hsa-miR-1307-3p and hsa-miR-3195);
(85-1) a combination of SEQ ID NOs: 2 and 5 (markers: hsa-miR-4730 and hsa-miR-663a);
(85-2) a combination of SEQ ID NOs: 1 and 5 (markers: hsa-miR-4783-3p and hsa-miR-663a);
(85-3) a combination of SEQ ID NOs: 237 and 5 (markers: hsa-miR-602 and hsa-miR-663a);
(85-4) a combination of SEQ ID NOs: 4 and 5 (markers: hsa-miR-4634 and hsa-miR-663a);
(85-5) a combination of SEQ ID NOs: 3 and 5 (markers: hsa-miR-1307-3p and hsa-miR-663a);
(86-1) a combination of SEQ ID NOs: 2 and 851 (markers: hsa-miR-4730 and hsa-miR-6089);
(86-2) a combination of SEQ ID NOs: 1 and 851 (markers: hsa-miR-4783-3p and hsa-miR-6089);
(86-3) a combination of SEQ ID NOs: 237 and 851 (markers: hsa-miR-602 and hsa-miR-6089);
(86-4) a combination of SEQ ID NOs: 4 and 851 (markers: hsa-miR-4634 and hsa-miR-6089);
(86-5) a combination of SEQ ID NOs: 3 and 851 (markers: hsa-miR-1307-3p and hsa-miR-6089);
(87-1) a combination of SEQ ID NOs: 2 and 852 (markers: hsa-miR-4730 and hsa-miR-6816-5p);
(87-2) a combination of SEQ ID NOs: 1 and 852 (markers: hsa-miR-4783-3p and hsa-miR-6816-5p);
(87-3) a combination of SEQ ID NOs: 237 and 852 (markers: hsa-miR-602 and hsa-miR-6816-5p);
(87-4) a combination of SEQ ID NOs: 4 and 852 (markers: hsa-miR-4634 and hsa-miR-6816-5p);
(87-5) a combination of SEQ ID NOs: 3 and 852 (markers: hsa-miR-1307-3p and hsa-miR-6816-5p);
(88-1) a combination of SEQ ID NOs: 2 and 30 (markers: hsa-miR-4730 and hsa-miR-6756-5p);
(88-2) a combination of SEQ ID NOs: 1 and 30 (markers: hsa-miR-4783-3p and hsa-miR-6756-5p);
(88-3) a combination of SEQ ID NOs: 237 and 30 (markers: hsa-miR-602 and hsa-miR-6756-5p);
(88-4) a combination of SEQ ID NOs: 4 and 30 (markers: hsa-miR-4634 and hsa-miR-6756-5p);
(88-5) a combination of SEQ ID NOs: 3 and 30 (markers: hsa-miR-1307-3p and hsa-miR-6756-5p);
(89-1) a combination of SEQ ID NOs: 2 and 93 (markers: hsa-miR-4730 and hsa-miR-6861-5p);
(89-2) a combination of SEQ ID NOs: 1 and 93 (markers: hsa-miR-4783-3p and hsa-miR-6861-5p);
(89-3) a combination of SEQ ID NOs: 237 and 93 (markers: hsa-miR-602 and hsa-miR-6861-5p);
(89-4) a combination of SEQ ID NOs: 4 and 93 (markers: hsa-miR-4634 and hsa-miR-6861-5p);
(89-5) a combination of SEQ ID NOs: 3 and 93 (markers: hsa-miR-1307-3p and hsa-miR-6861-5p);
(90-1) a combination of SEQ ID NOs: 2 and 27 (markers: hsa-miR-4730 and hsa-miR-1246);
(90-2) a combination of SEQ ID NOs: 1 and 27 (markers: hsa-miR-4783-3p and hsa-miR-1246);
(90-3) a combination of SEQ ID NOs: 237 and 27 (markers: hsa-miR-602 and hsa-miR-1246);
(90-4) a combination of SEQ ID NOs: 4 and 27 (markers: hsa-miR-4634 and hsa-miR-1246);
(90-5) a combination of SEQ ID NOs: 27 and 208 (markers: hsa-miR-1246 and hsa-miR-1343-5p);
(91-1) a combination of SEQ ID NOs: 2 and 853 (markers: hsa-miR-4730 and hsa-miR-4466);
(91-2) a combination of SEQ ID NOs: 1 and 853 (markers: hsa-miR-4783-3p and hsa-miR-4466);
(91-3) a combination of SEQ ID NOs: 237 and 853 (markers: hsa-miR-602 and hsa-miR-4466);
(91-4) a combination of SEQ ID NOs: 4 and 853 (markers: hsa-miR-4634 and hsa-miR-4466);
(91-5) a combination of SEQ ID NOs: 3 and 853 (markers: hsa-miR-1307-3p and hsa-miR-4466);
(92-1) a combination of SEQ ID NOs: 2 and 238 (markers: hsa-miR-4730 and hsa-miR-423-5p);
(92-2) a combination of SEQ ID NOs: 1 and 238 (markers: hsa-miR-4783-3p and hsa-miR-423-5p);
(92-3) a combination of SEQ ID NOs: 237 and 238 (markers: hsa-miR-602 and hsa-miR-423-5p);
(92-4) a combination of SEQ ID NOs: 4 and 238 (markers: hsa-miR-4634 and hsa-miR-423-5p);
(92-5) a combination of SEQ ID NOs: 3 and 238 (markers: hsa-miR-1307-3p and hsa-miR-423-5p);
(93-1) a combination of SEQ ID NOs: 2 and 130 (markers: hsa-miR-4730 and hsa-miR-6075);
(93-2) a combination of SEQ ID NOs: 1 and 130 (markers: hsa-miR-4783-3p and hsa-miR-6075);
(93-3) a combination of SEQ ID NOs: 237 and 130 (markers: hsa-miR-602 and hsa-miR-6075);
(93-4) a combination of SEQ ID NOs: 4 and 130 (markers: hsa-miR-4634 and hsa-miR-6075);
(93-5) a combination of SEQ ID NOs: 3 and 130 (markers: hsa-miR-1307-3p and hsa-miR-6075);
(94-1) a combination of SEQ ID NOs: 2 and 177 (markers: hsa-miR-4730 and hsa-miR-7108-5p);
(94-2) a combination of SEQ ID NOs: 1 and 177 (markers: hsa-miR-4783-3p and hsa-miR-7108-5p);
(94-3) a combination of SEQ ID NOs: 237 and 177 (markers: hsa-miR-602 and hsa-miR-7108-5p);
(94-4) a combination of SEQ ID NOs: 4 and 177 (markers: hsa-miR-4634 and hsa-miR-7108-5p);
(94-5) a combination of SEQ ID NOs: 3 and 177 (markers: hsa-miR-1307-3p and hsa-miR-7108-5p);
(95-1) a combination of SEQ ID NOs: 2 and 64 (markers: hsa-miR-4730 and hsa-miR-6511a-5p);
(95-2) a combination of SEQ ID NOs: 1 and 64 (markers: hsa-miR-4783-3p and hsa-miR-6511a-5p);
(95-3) a combination of SEQ ID NOs: 237 and 64 (markers: hsa-miR-602 and hsa-miR-6511a-5p);
(95-4) a combination of SEQ ID NOs: 4 and 64 (markers: hsa-miR-4634 and hsa-miR-6511a-5p);
(95-5) a combination of SEQ ID NOs: 3 and 64 (markers: hsa-miR-1307-3p and hsa-miR-6511a-5p);
(96-1) a combination of SEQ ID NOs: 2 and 114 (markers: hsa-miR-4730 and hsa-miR-211-3p);
(96-2) a combination of SEQ ID NOs: 1 and 114 (markers: hsa-miR-4783-3p and hsa-miR-211-3p);
(96-3) a combination of SEQ ID NOs: 237 and 114 (markers: hsa-miR-602 and hsa-miR-211-3p);
(96-4) a combination of SEQ ID NOs: 4 and 114 (markers: hsa-miR-4634 and hsa-miR-211-3p);
(96-5) a combination of SEQ ID NOs: 3 and 114 (markers: hsa-miR-1307-3p and hsa-miR-211-3p);
(97-1) a combination of SEQ ID NOs: 2 and 119 (markers: hsa-miR-4730 and hsa-miR-7110-5p);
(97-2) a combination of SEQ ID NOs: 1 and 119 (markers: hsa-miR-4783-3p and hsa-miR-7110-5p);
(97-3) a combination of SEQ ID NOs: 237 and 119 (markers: hsa-miR-602 and hsa-miR-7110-5p);
(97-4) a combination of SEQ ID NOs: 4 and 119 (markers: hsa-miR-4634 and hsa-miR-7110-5p);
(97-5) a combination of SEQ ID NOs: 3 and 119 (markers: hsa-miR-1307-3p and hsa-miR-7110-5p);
(98-1) a combination of SEQ ID NOs: 2 and 135 (markers: hsa-miR-4730 and hsa-miR-6870-5p);
(98-2) a combination of SEQ ID NOs: 1 and 135 (markers: hsa-miR-4783-3p and hsa-miR-6870-5p);
(98-3) a combination of SEQ ID NOs: 237 and 135 (markers: hsa-miR-602 and hsa-miR-6870-5p);
(98-4) a combination of SEQ ID NOs: 4 and 135 (markers: hsa-miR-4634 and hsa-miR-6870-5p);
(98-5) a combination of SEQ ID NOs: 3 and 135 (markers: hsa-miR-1307-3p and hsa-miR-6870-5p);
(99-1) a combination of SEQ ID NOs: 2 and 243 (markers: hsa-miR-4730 and hsa-miR-486-5p);
(99-2) a combination of SEQ ID NOs: 1 and 243 (markers: hsa-miR-4783-3p and hsa-miR-486-5p);
(99-3) a combination of SEQ ID NOs: 237 and 243 (markers: hsa-miR-602 and hsa-miR-486-5p);
(99-4) a combination of SEQ ID NOs: 4 and 243 (markers: hsa-miR-4634 and hsa-miR-486-5p);
(99-5) a combination of SEQ ID NOs: 3 and 243 (markers: hsa-miR-1307-3p and hsa-miR-486-5p);
(100-1) a combination of SEQ ID NOs: 2 and 122 (markers: hsa-miR-4730 and hsa-miR-4792);
(100-2) a combination of SEQ ID NOs: 1 and 122 (markers: hsa-miR-4783-3p and hsa-miR-4792);
(100-3) a combination of SEQ ID NOs: 237 and 122 (markers: hsa-miR-602 and hsa-miR-4792);
(100-4) a combination of SEQ ID NOs: 4 and 122 (markers: hsa-miR-4634 and hsa-miR-4792);
(100-5) a combination of SEQ ID NOs: 3 and 122 (markers: hsa-miR-1307-3p and hsa-miR-4792);
(101-1) a combination of SEQ ID NOs: 2 and 260 (markers: hsa-miR-4730 and hsa-miR-4687-3p);
(101-2) a combination of SEQ ID NOs: 1 and 260 (markers: hsa-miR-4783-3p and hsa-miR-4687-3p);
(101-3) a combination of SEQ ID NOs: 237 and 260 (markers: hsa-miR-602 and hsa-miR-4687-3p);
(101-4) a combination of SEQ ID NOs: 4 and 260 (markers: hsa-miR-4634 and hsa-miR-4687-3p);
(101-5) a combination of SEQ ID NOs: 3 and 260 (markers: hsa-miR-1307-3p and hsa-miR-4687-3p);
(102-1) a combination of SEQ ID NOs: 2 and 59 (markers: hsa-miR-4730 and hsa-miR-1238-5p);
(102-2) a combination of SEQ ID NOs: 1 and 59 (markers: hsa-miR-4783-3p and hsa-miR-1238-5p);
(102-3) a combination of SEQ ID NOs: 237 and 59 (markers: hsa-miR-602 and hsa-miR-1238-5p);
(102-4) a combination of SEQ ID NOs: 4 and 59 (markers: hsa-miR-4634 and hsa-miR-1238-5p);
(102-5) a combination of SEQ ID NOs: 3 and 59 (markers: hsa-miR-1307-3p and hsa-miR-1238-5p);
(103-1) a combination of SEQ ID NOs: 2 and 854 (markers: hsa-miR-4730 and hsa-miR-4488);
(103-2) a combination of SEQ ID NOs: 1 and 854 (markers: hsa-miR-4783-3p and hsa-miR-4488);
(103-3) a combination of SEQ ID NOs: 237 and 854 (markers: hsa-miR-602 and hsa-miR-4488);
(103-4) a combination of SEQ ID NOs: 4 and 854 (markers: hsa-miR-4634 and hsa-miR-4488);
(103-5) a combination of SEQ ID NOs: 3 and 854 (markers: hsa-miR-1307-3p and hsa-miR-4488);
(104-1) a combination of SEQ ID NOs: 2 and 132 (markers: hsa-miR-4730 and hsa-miR-6891-5p);
(104-2) a combination of SEQ ID NOs: 1 and 132 (markers: hsa-miR-4783-3p and hsa-miR-6891-5p);
(104-3) a combination of SEQ ID NOs: 237 and 132 (markers: hsa-miR-602 and hsa-miR-6891-5p);
(104-4) a combination of SEQ ID NOs: 4 and 132 (markers: hsa-miR-4634 and hsa-miR-6891-5p);
(104-5) a combination of SEQ ID NOs: 3 and 132 (markers: hsa-miR-1307-3p and hsa-miR-6891-5p);
(105-1) a combination of SEQ ID NOs: 2 and 181 (markers: hsa-miR-4730 and hsa-miR-2861);
(105-2) a combination of SEQ ID NOs: 1 and 181 (markers: hsa-miR-4783-3p and hsa-miR-2861);
(105-3) a combination of SEQ ID NOs: 237 and 181 (markers: hsa-miR-602 and hsa-miR-2861);
(105-4) a combination of SEQ ID NOs: 4 and 181 (markers: hsa-miR-4634 and hsa-miR-2861);
(105-5) a combination of SEQ ID NOs: 3 and 181 (markers: hsa-miR-1307-3p and hsa-miR-2861);
(106-1) a combination of SEQ ID NOs: 2 and 79 (markers: hsa-miR-4730 and hsa-miR-4665-5p);
(106-2) a combination of SEQ ID NOs: 1 and 79 (markers: hsa-miR-4783-3p and hsa-miR-4665-5p);
(106-3) a combination of SEQ ID NOs: 237 and 79 (markers: hsa-miR-602 and hsa-miR-4665-5p);
(106-4) a combination of SEQ ID NOs: 4 and 79 (markers: hsa-miR-4634 and hsa-miR-4665-5p);
(106-5) a combination of SEQ ID NOs: 3 and 79 (markers: hsa-miR-1307-3p and hsa-miR-4665-5p);
(107-1) a combination of SEQ ID NOs: 2 and 133 (markers: hsa-miR-4730 and hsa-miR-4745-5p);
(107-2) a combination of SEQ ID NOs: 1 and 133 (markers: hsa-miR-4783-3p and hsa-miR-4745-5p);
(107-3) a combination of SEQ ID NOs: 237 and 133 (markers: hsa-miR-602 and hsa-miR-4745-5p);
(107-4) a combination of SEQ ID NOs: 4 and 133 (markers: hsa-miR-4634 and hsa-miR-4745-5p);
(107-5) a combination of SEQ ID NOs: 3 and 133 (markers: hsa-miR-1307-3p and hsa-miR-4745-5p);
(108-1) a combination of SEQ ID NOs: 2 and 41 (markers: hsa-miR-4730 and hsa-miR-6858-5p);
(108-2) a combination of SEQ ID NOs: 1 and 41 (markers: hsa-miR-4783-3p and hsa-miR-6858-5p);
(108-3) a combination of SEQ ID NOs: 237 and 41 (markers: hsa-miR-602 and hsa-miR-6858-5p);
(108-4) a combination of SEQ ID NOs: 4 and 41 (markers: hsa-miR-4634 and hsa-miR-6858-5p);
(108-5) a combination of SEQ ID NOs: 3 and 41 (markers: hsa-miR-1307-3p and hsa-miR-6858-5p);
(109-1) a combination of SEQ ID NOs: 2 and 139 (markers: hsa-miR-4730 and hsa-miR-6825-5p);
(109-2) a combination of SEQ ID NOs: 1 and 139 (markers: hsa-miR-4783-3p and hsa-miR-6825-5p);
(109-3) a combination of SEQ ID NOs: 237 and 139 (markers: hsa-miR-602 and hsa-miR-6825-5p);
(109-4) a combination of SEQ ID NOs: 4 and 139 (markers: hsa-miR-4634 and hsa-miR-6825-5p);
(109-5) a combination of SEQ ID NOs: 3 and 139 (markers: hsa-miR-1307-3p and hsa-miR-6825-5p);
(110-1) a combination of SEQ ID NOs: 2 and 118 (markers: hsa-miR-4730 and hsa-miR-614);
(110-2) a combination of SEQ ID NOs: 1 and 118 (markers: hsa-miR-4783-3p and hsa-miR-614);
(110-3) a combination of SEQ ID NOs: 237 and 118 (markers: hsa-miR-602 and hsa-miR-614);
(110-4) a combination of SEQ ID NOs: 4 and 118 (markers: hsa-miR-4634 and hsa-miR-614);
(110-5) a combination of SEQ ID NOs: 3 and 118 (markers: hsa-miR-1307-3p and hsa-miR-614);
(111-1) a combination of SEQ ID NOs: 2 and 86 (markers: hsa-miR-4730 and hsa-miR-1343-3p);
(111-2) a combination of SEQ ID NOs: 1 and 86 (markers: hsa-miR-4783-3p and hsa-miR-1343-3p);
(111-3) a combination of SEQ ID NOs: 237 and 86 (markers: hsa-miR-602 and hsa-miR-1343-3p);
(111-4) a combination of SEQ ID NOs: 4 and 86 (markers: hsa-miR-4634 and hsa-miR-1343-3p);
(111-5) a combination of SEQ ID NOs: 3 and 86 (markers: hsa-miR-1307-3p and hsa-miR-1343-3p);
(112-1) a combination of SEQ ID NOs: 2 and 60 (markers: hsa-miR-4730 and hsa-miR-365a-5p);
(112-2) a combination of SEQ ID NOs: 1 and 60 (markers: hsa-miR-4783-3p and hsa-miR-365a-5p);
(112-3) a combination of SEQ ID NOs: 237 and 60 (markers: hsa-miR-602 and hsa-miR-365a-5p);
(112-4) a combination of SEQ ID NOs: 4 and 60 (markers: hsa-miR-4634 and hsa-miR-365a-5p);
(112-5) a combination of SEQ ID NOs: 3 and 60 (markers: hsa-miR-1307-3p and hsa-miR-365a-5p);
(113-1) a combination of SEQ ID NOs: 2 and 116 (markers: hsa-miR-4730 and hsa-miR-4750-5p);
(113-2) a combination of SEQ ID NOs: 1 and 116 (markers: hsa-miR-4783-3p and hsa-miR-4750-5p);
(113-3) a combination of SEQ ID NOs: 237 and 116 (markers: hsa-miR-602 and hsa-miR-4750-5p);
(113-4) a combination of SEQ ID NOs: 4 and 116 (markers: hsa-miR-4634 and hsa-miR-4750-5p);
(113-5) a combination of SEQ ID NOs: 3 and 116 (markers: hsa-miR-1307-3p and hsa-miR-4750-5p);
(114-1) a combination of SEQ ID NOs: 2 and 160 (markers: hsa-miR-4730 and hsa-miR-6732-5p);
(114-2) a combination of SEQ ID NOs: 1 and 160 (markers: hsa-miR-4783-3p and hsa-miR-6732-5p);
(114-3) a combination of SEQ ID NOs: 237 and 160 (markers: hsa-miR-602 and hsa-miR-6732-5p);
(114-4) a combination of SEQ ID NOs: 4 and 160 (markers: hsa-miR-4634 and hsa-miR-6732-5p);
(114-5) a combination of SEQ ID NOs: 3 and 160 (markers: hsa-miR-1307-3p and hsa-miR-6732-5p);
(115-1) a combination of SEQ ID NOs: 2 and 38 (markers: hsa-miR-4730 and hsa-miR-149-3p);
(115-2) a combination of SEQ ID NOs: 1 and 38 (markers: hsa-miR-4783-3p and hsa-miR-149-3p);
(115-3) a combination of SEQ ID NOs: 237 and 38 (markers: hsa-miR-602 and hsa-miR-149-3p);
(115-4) a combination of SEQ ID NOs: 4 and 38 (markers: hsa-miR-4634 and hsa-miR-149-3p);
(115-5) a combination of SEQ ID NOs: 3 and 38 (markers: hsa-miR-1307-3p and hsa-miR-149-3p);
(116-1) a combination of SEQ ID NOs: 2 and 99 (markers: hsa-miR-4730 and hsa-miR-4497);
(116-2) a combination of SEQ ID NOs: 1 and 99 (markers: hsa-miR-4783-3p and hsa-miR-4497);
(116-3) a combination of SEQ ID NOs: 237 and 99 (markers: hsa-miR-602 and hsa-miR-4497);
(116-4) a combination of SEQ ID NOs: 4 and 99 (markers: hsa-miR-4634 and hsa-miR-4497);
(116-5) a combination of SEQ ID NOs: 3 and 99 (markers: hsa-miR-1307-3p and hsa-miR-4497);
(117-1) a combination of SEQ ID NOs: 2 and 104 (markers: hsa-miR-4730 and hsa-miR-6848-5p);
(117-2) a combination of SEQ ID NOs: 1 and 104 (markers: hsa-miR-4783-3p and hsa-miR-6848-5p);
(117-3) a combination of SEQ ID NOs: 237 and 104 (markers: hsa-miR-602 and hsa-miR-6848-5p);
(117-4) a combination of SEQ ID NOs: 4 and 104 (markers: hsa-miR-4634 and hsa-miR-6848-5p); and (117-5) a combination of SEQ ID NOs: 3 and 104 (markers: hsa-miR-1307-3p and hsa-miR-6848-5p).

The kit or the device of the present invention may also comprise a polynucleotide(s) that is/are already known or that will be found in the future, to enable detection of breast cancer, in addition to the polynucleotide(s) (which may include variant(s), fragment(s), or derivative(s)) according to the present invention

The kit of the present invention may also comprise an antibody for measuring marker(s) for breast cancer examination known in the art, such as CEA, CA-15-3, and CA27-29, in addition to the polynucleotide(s) as described above.

The polynucleotides described above contained in the kit of the present invention may be packaged in different containers either individually or in any combination.

The kit of the present invention may comprise a kit for extracting nucleic acids (e.g., total RNA) from body fluids, cells, or tissues, a fluorescent material for labeling, an enzyme and a medium for nucleic acid amplification, an instruction manual, etc.

The device of the present invention is a device for cancer marker measurement in which nucleic acids such as the polynucleotides according to the present invention described above, variants thereof, derivatives thereof, or fragments thereof are bound or attached to, for example, a solid phase. Examples of the material for the solid phase include plastics, paper, glass, and silicon. The material for the solid phase is preferably a plastic from the viewpoint of easy processability. The solid phase has any shape and is, for example, square, round, reed-shaped, or film-shaped. The device of the present invention includes, for example, a device for measurement by a hybridization technique. Specific examples thereof include blotting devices and nucleic acid arrays (e.g., microarrays, DNA chips, and RNA chips).

The nucleic acid array technique is a technique which involves binding or attaching the nucleic acids one by one by use of a method [e.g., a method of spotting the nucleic acids using a high-density dispenser called spotter or arrayer onto the surface of the solid phase surface-treated, if necessary, by coating with L-lysine or the introduction of a functional group such as an amino group or a carboxyl group; a method of spraying the nucleic acids onto the solid phase using an inkjet which injects very small liquid droplets by a piezoelectric element or the like from a nozzle; or a method of sequentially synthesizing nucleotides on the solid phase] to prepare an array such as a chip and measuring a target nucleic acid through the use of hybridization using this array.

The kit or the device of the present invention comprises nucleic acids capable of specifically binding to the polynucleotides of at least one, preferably at least two, more preferably at least three, most preferably at least five to all of the breast cancer marker miRNAs, respectively, of the group 1 described above. The kit or the device of the present invention may optionally further comprise nucleic acids capable of specifically binding to the polynucleotides of at least one, preferably at least two, more preferably at least three, most preferably at least five to all of the breast cancer marker miRNAs, respectively, of the group 2 described above. The kit or the device of the present invention may optionally further comprise nucleic acids capable of specifically binding to the polynucleotides of at least one, preferably at least two, more preferably at least three, most preferably at least five to all of the breast cancer marker miRNAs, respectively, of the group 3 described above. The kit or the device of the present invention can be used for detecting breast cancer as described in Section 4 below.

4. Method for detecting breast cancer

The present invention further provides a method for detecting breast cancer, comprising using the kit or the device of the present invention (comprising the aforementioned nucleic acid(s) that can be used in the present invention) as described in Section “3. Kit or device for detection of breast cancer” to measure an expression level of one or more breast cancer-derived genes represented by: expression level(s) of breast cancer-derived genes selected from the following group of miRNAs, i.e., miR-4783-3p, miR-4730, miR-1307-3p, miR-4634, miR-663a, miR-4532, miR-7704, miR-3178, miR-6729-5p, miR-6090, miR-4732-5p, miR-3184-5p, miR-6727-5p, miR-6088, miR-4674, miR-8073, miR-4787-5p, miR-1469, miR-125a-3p, miR-1233-5p, miR-885-3p, miR-6802-5p, miR-328-5p, miR-6787-5p, miR-8069, miR-6875-5p, miR-1246, miR-4734, miR-6757-5p, miR-6756-5p, miR-3665, miR-6836-3p, miR-6821-5p, miR-6805-5p, miR-4728-5p, miR-6726-5p, miR-197-5p, miR-149-3p, miR-6850-5p, miR-4476, miR-6858-5p, miR-564, miR-4763-3p, miR-575, miR-6771-5p, miR-1231, miR-1908-3p, miR-150-3p, miR-3937, miR-887-3p, miR-3940-5p, miR-4741, miR-6808-5p, miR-6869-5p, miR-5090, miR-615-5p, miR-8072, miR-128-1-5p, miR-1238-5p, miR-365a-5p, miR-204-3p, miR-4492, miR-6785-5p, miR-6511a-5p, miR-4525, miR-1915-5p, miR-3180, miR-6879-5p, miR-1199-5p, miR-6746-5p, miR-711, miR-663b, miR-4707-3p, miR-6893-5p, miR-4675, miR-4638-5p, miR-4651, miR-6087, miR-4665-5p, miR-4758-5p, miR-6887-5p, miR-3620-5p, miR-1909-3p, miR-7641, miR-6724-5p, miR-1343-3p, miR-6780b-5p, miR-4484, miR-4690-5p, miR-4429, miR-1227-5p, miR-4725-3p, miR-6861-5p, miR-6812-5p, miR-3197, miR-8059, miR-3185, miR-4706, miR-4497, miR-3131, miR-6806-5p, miR-187-5p, miR-3180-3p, miR-6848-5p, miR-6820-5p, miR-6800-5p, miR-6717-5p, miR-6795-5p, miR-4632-5p, miR-665, miR-6778-5p, miR-3663-3p, miR-4689, miR-211-3p, miR-6511b-5p, miR-4750-5p, miR-6126, miR-614, miR-7110-5p, miR-744-5p, miR-6769a-5p, miR-4792, miR-5787, miR-6798-5p, miR-6781-5p, miR-4419b, miR-4446-3p, miR-4259, miR-5572, miR-6075, miR-296-3p, miR-6891-5p, miR-4745-5p, miR-6775-5p, miR-6870-5p, miR-920, miR-4530, miR-6819-5p, miR-6825-5p, miR-7847-3p, miR-6131, miR-4433-3p, miR-1228-5p, miR-6743-5p, miR-1268a, miR-3917, miR-6786-5p, miR-3154, miR-638, miR-6741-5p, miR-6889-5p, miR-6840-3p, miR-6510-5p, miR-3188, miR-551b-5p, miR-5001-5p, miR-1268b, miR-7107-5p, miR-6824-5p, miR-6732-5p, miR-371a-5p, miR-6794-5p, miR-6779-5p, miR-4271, miR-5195-3p, miR-6762-5p, miR-939-5p, miR-1247-3p, miR-6777-5p, miR-6722-3p, miR-3656, miR-4688, miR-3195, miR-6766-5p, miR-4447, miR-4656, miR-7108-5p, miR-3191-3p, miR-1273g-3p, miR-4463, miR-2861, miR-3196, miR-6877-5p, miR-3679-5p, miR-4442, miR-6789-5p, miR-6782-5p, miR-486-3p, miR-6085, miR-4746-3p, miR-619-5p, miR-937-5p, miR-6803-5p, miR-4298, miR-4454, miR-4459, miR-7150, miR-6880-5p, miR-4449, miR-8063, miR-4695-5p, miR-6132, miR-6829-5p, miR-4486, miR-6805-3p, miR-6826-5p, miR-4508, miR-1343-5p, miR-7114-5p, miR-3622a-5p, miR-6765-5p, miR-7845-5p, miR-3960, miR-6749-5p, miR-1260b, miR-6799-5p, miR-4723-5p, miR-6784-5p, miR-5100, miR-6769b-5p, miR-1207-5p, miR-642a-3p, miR-4505, miR-4270, miR-6721-5p, miR-7111-5p, miR-6791-5p, miR-7109-5p, miR-4258, miR-6515-3p, miR-6851-5p, miR-6125, miR-4749-5p, miR-4726-5p, miR-4513, miR-6089, miR-6816-5p, miR-4466, miR-4488, miR-6752-5p and miR-4739; and optionally expression level(s) of breast cancer-derived gene(s) selected from the following group of miRNA: i.e., miR-760, miR-602, miR-423-5p, miR-92a-2-5p, miR-16-5p, miR-451a, miR-135a-3p, miR-486-5p, miR-4257, miR-92b-5p, miR-1915-3p, miR-718, miR-940, miR-296-5p, miR-23b-3p and miR-92a-3p, and optionally expression level(s) of breast cancer-derived gene(s) selected from the following group of miRNA: i.e., miR-658, miR-6842-5p, miR-6124, miR-6765-3p, miR-7106-5p, miR-4534, miR-92b-3p, miR-3135b, miR-4687-3p, miR-762, miR-3619-3p, miR-4467, miR-557, miR-1237-5p, miR-1908-5p, miR-4286, miR-6885-5p, and miR-6763-5p in a sample in vitro, further comparing, for example, the expression level(s) of the aforementioned gene(s) in the sample (e.g., blood, serum, or plasma) collected from a subject suspected of having breast cancer with a control expression level in the sample collected from a healthy subject (including a non-breast cancer patient(s)), and evaluating the subject as having breast cancer when the expression level(s) of the target nucleic acid(s) is different between the samples.

This method of the present invention enables limitedly-invasive early diagnosis of the breast cancer with high sensitivity and specificity and thereby brings about early treatment and improved prognosis. In addition, exacerbation of the disease or the effectiveness of surgical, radiotherapeutic, and chemotherapeutic treatments can be monitored.

The method for extracting the breast cancer-derived gene from the sample such as blood, serum, or plasma according to the present invention is particularly preferably a method in which the breast cancer-derived gene is prepared by the addition of a reagent for RNA extraction in 3D-Gener™ RNA extraction reagent from liquid sample kit (Toray Industries, Inc.). A general acidic phenol method (acid guanidinium-phenol-chloroform (AGPC)) may be used, or Trizol™ (Life Technologies Corp.) may be used. The breast cancer-derived gene(s) may be prepared by the addition of a reagent for RNA extraction containing acidic phenol, such as Trizol (Life Technologies Corp.) or Isogen (Nippon Gene Co., Ltd., Japan). Alternatively, a kit such as miRNeasy® Mini Kit (Qiagen N.V.) can be used, though the method is not limited thereto.

The present invention also provides use of the kit or the device of the present invention for detecting in vitro an expression product(s) of a breast cancer-derived miRNA gene in a sample derived from a subject.

In the method of the present invention, the used kit or the device comprises a single polynucleotide or any possible combination of polynucleotides that can be used in the present invention as described above.

In the detection or (genetic) diagnosis of breast cancer according to the present invention, each polynucleotide contained in the kit or the device of the present invention can be used as a probe or a primer. In the case of using the polynucleotide as a primer, TaqMan™ MicroRNA Assays from Life Technologies Corp., miScript PCR System from Qiagen N.V., or the like can be used, though the method is not limited thereto.

The polynucleotide contained in the kit or the device of the present invention can be used as a primer or a probe according to a routine method in a method known in the art for specifically detecting the particular gene, for example, a hybridization technique such as Northern blot, Southern blot, in situ hybridization, Northern hybridization, or Southern hybridization, or a quantitative amplification technique such as quantitative RT-PCR. A body fluid such as blood, serum, plasma, or urine from a subject is collected as a sample to be assayed according to the type of the detection method used. Alternatively, total RNA prepared from such a body fluid by the method described above may be used, and various polynucleotides including cDNA prepared on the basis of the RNA may be used.

The kit or the device of the present invention is useful for the diagnosis of breast or the detection of the presence or absence of breast cancer. Specifically, the detection of breast cancer using the kit or the device can be performed by detecting in vitro an expression level of a gene using the nucleic acid probe or the primer contained in the kit or the device in a sample such as blood, serum, plasma, or urine from a subject suspected of having breast cancer. The subject suspected of having breast cancer can be evaluated as having breast cancer when the expression level(s) of a target miRNA marker(s) measured using a polynucleotide(s) (including variant(s), fragment(s), or derivative(s) thereof) consisting of a nucleotide sequence(s) represented by at least one of SEQ ID NOs: 1 to 235, and 851 to 856 or a complementary sequence(s) thereof, and optionally a nucleotide sequence(s) represented by one or more of SEQ ID NOs: 236 to 251 or a complementary sequence(s) thereof, and optionally a nucleotide sequence(s) represented by one or more of SEQ ID NOs: 252 to 269 or a complementary sequence thereof in the sample such as blood, serum, plasma, or urine of the subject has a statistically significantly higher or lower than the expression level(s) thereof in the sample such as blood, serum, or plasma, or urine of a healthy subject.

The method of the present invention can be combined with a diagnostic imaging method such as mammography, ultrasonography (echo examination), CT, MRI, abdominal ultrasonography, bone scintigraphy, or PET, or pathological examination which involves analyzing a lesion tissue under a microscope. The method of the present invention is capable of specifically detecting breast cancer and can substantially discriminate breast cancer from the other cancers.

The method for detecting the absence of an expression product(s) of a breast cancer-derived gene(s) or the presence of the expression product(s) of a breast cancer-derived gene in a sample using the kit or the device of the present invention comprises; collecting a body fluid such as blood, serum, plasma, or urine of a subject; measuring the expression level(s) of the target gene(s) contained therein using one or more polynucleotides (including variant(s), fragment(s), or derivative(s)) selected from the polynucleotide group of the present invention; and evaluating the presence or absence of breast cancer or to detect breast cancer. The method for detecting breast cancer according to the present invention can also evaluate or diagnose, for example, the presence or absence of amelioration of the disease or the degree of amelioration thereof in a breast cancer patient in the case that a therapeutic drug is administered to the patient for amelioration of the disease.

The method of the present invention can comprise, for example, the following steps (a), (b), and (c):
(a) a step of contacting in vitro a sample from a subject with a polynucleotide(s) contained in the kit or the device of the present invention in vitro;
(b) a step of measuring an expression level(s) of the target nucleic acid(s) in the sample using the polynucleotide(s) as a nucleic acid probe(s) or a primer(s); and
(c) a step of evaluating the presence or absence of breast cancer (cells) in the subject on the basis of the measurement results in the step (b).

Specifically, the present invention provides a method for detecting breast cancer, comprising measuring an expression level(s) of a target nucleic acid(s) in a sample of a subject using nucleic acid(s) capable of specifically binding to at least one (preferably at least two) polynucleotides selected from the group consisting of miR-4783-3p, miR-4730, miR-1307-3p, miR-4634, miR-663a, miR-4532, miR-7704, miR-3178, miR-6729-5p, miR-6090, miR-4732-5p, miR-3184-5p, miR-6727-5p, miR-6088, miR-4674, miR-8073, miR-4787-5p, miR-1469, miR-125a-3p, miR-1233-5p, miR-885-3p, miR-6802-5p, miR-328-5p, miR-6787-5p, miR-8069, miR-6875-5p, miR-1246, miR-4734, miR-6757-5p, miR-6756-5p, miR-3665, miR-6836-3p, miR-6821-5p, miR-6805-5p, miR-4728-5p, miR-6726-5p, miR-197-5p, miR-149-3p, miR-6850-5p, miR-4476, miR-6858-5p, miR-564, miR-4763-3p, miR-575, miR-6771-5p, miR-1231, miR-1908-3p, miR-150-3p, miR-3937, miR-887-3p, miR-3940-5p, miR-4741, miR-6808-5p, miR-6869-5p, miR-5090, miR-615-5p, miR-8072, miR-128-1-5p, miR-1238-5p, miR-365a-5p, miR-204-3p, miR-4492, miR-6785-5p, miR-6511a-5p, miR-4525, miR-1915-5p, miR-3180, miR-6879-5p, miR-1199-5p, miR-6746-5p, miR-711, miR-663b, miR-4707-3p, miR-6893-5p, miR-4675, miR-4638-5p, miR-4651, miR-6087, miR-4665-5p, miR-4758-5p, miR-6887-5p, miR-3620-5p, miR-1909-3p, miR-7641, miR-6724-5p, miR-1343-3p, miR-6780b-5p, miR-4484, miR-4690-5p, miR-4429, miR-1227-5p, miR-4725-3p, miR-6861-5p, miR-6812-5p, miR-3197, miR-8059, miR-3185, miR-4706, miR-4497, miR-3131, miR-6806-5p, miR-187-5p, miR-3180-3p, miR-6848-5p, miR-6820-5p, miR-6800-5p, miR-6717-5p, miR-6795-5p, miR-4632-5p, miR-665, miR-6778-5p, miR-3663-3p, miR-4689, miR-211-3p, miR-6511b-5p, miR-4750-5p, miR-6126, miR-614, miR-7110-5p, miR-744-5p, miR-6769a-5p, miR-4792, miR-5787, miR-6798-5p, miR-6781-5p, miR-4419b, miR-4446-3p, miR-4259, miR-5572, miR-6075, miR-296-3p, miR-6891-5p, miR-4745-5p, miR-6775-5p, miR-6870-5p, miR-920, miR-4530, miR-6819-5p, miR-6825-5p, miR-7847-3p, miR-6131, miR-4433-3p, miR-1228-5p, miR-6743-5p, miR-1268a, miR-3917, miR-6786-5p, miR-3154, miR-638, miR-6741-5p, miR-6889-5p, miR-6840-3p, miR-6510-5p, miR-3188, miR-551b-5p, miR-5001-5p, miR-1268b, miR-7107-5p, miR-6824-5p, miR-6732-5p, miR-371a-5p, miR-6794-5p, miR-6779-5p, miR-4271, miR-5195-3p, miR-6762-5p, miR-939-5p, miR-1247-3p, miR-6777-5p, miR-6722-3p, miR-3656, miR-4688, miR-3195, miR-6766-5p, miR-4447, miR-4656, miR-7108-5p, miR-3191-3p, miR-1273g-3p, miR-4463, miR-2861, miR-3196, miR-6877-5p, miR-3679-5p, miR-4442, miR-6789-5p, miR-6782-5p, miR-486-3p, miR-6085, miR-4746-3p, miR-619-5p, miR-937-5p, miR-6803-5p, miR-4298, miR-4454, miR-4459, miR-7150, miR-6880-5p, miR-4449, miR-8063, miR-4695-5p, miR-6132, miR-6829-5p, miR-4486, miR-6805-3p, miR-6826-5p, miR-4508, miR-1343-5p, miR-7114-5p, miR-3622a-5p, miR-6765-5p, miR-7845-5p, miR-3960, miR-6749-5p, miR-1260b, miR-6799-5p, miR-4723-5p, miR-6784-5p, miR-5100, miR-6769b-5p, miR-1207-5p, miR-642a-3p, miR-4505, miR-4270, miR-6721-5p, miR-7111-5p, miR-6791-5p, miR-7109-5p, miR-4258, miR-6515-3p, miR-6851-5p, miR-6125, miR-4749-5p, miR-4726-5p, miR-4513, miR-6089, miR-6816-5p, miR-4466, miR-4488, miR-6752-5p and miR-4739 and evaluating in vitro whether or not the subject has breast cancer in the subject using the above-measured expression levels and control expression levels of healthy subjects measured in the same way as above.

The term “evaluation” used herein is evaluation support based on results of in vitro examination, not physician's judgment.

As described above, in a preferred embodiment of the method of the present invention, specifically, miR-4783-3p is hsa-miR-4783-3p, miR-4730 is hsa-miR-4730, miR-1307-3p is hsa-miR-1307-3p, miR-4634 is hsa-miR-4634, miR-663a is hsa-miR-663a, miR-4532 is hsa-miR-4532, miR-7704 is hsa-miR-7704, miR-3178 is hsa-miR-3178, miR-6729-5p is hsa-miR-6729-5p, miR-6090 is hsa-miR-6090, miR-4732-5p is hsa-miR-4732-5p, miR-3184-5p is hsa-miR-3184-5p, miR-6727-5p is hsa-miR-6727-5p, miR-6088 is hsa-miR-6088, miR-4674 is hsa-miR-4674, miR-8073 is hsa-miR-8073, miR-4787-5p is hsa-miR-4787-5p, miR-1469 is hsa-miR-1469, miR-125a-3p is hsa-miR-125a-3p, miR-1233-5p is hsa-miR-1233-5p, miR-885-3p is hsa-miR-885-3p, miR-6802-5p is hsa-miR-6802-5p, miR-328-5p is hsa-miR-328-5p, miR-6787-5p is hsa-miR-6787-5p, miR-8069 is hsa-miR-8069, miR-6875-5p is hsa-miR-6875-5p, miR-1246 is hsa-miR-1246, miR-4734 is hsa-miR-4734, miR-6757-5p is hsa-miR-6757-5p, miR-6756-5p is hsa-miR-6756-5p, miR-3665 is hsa-miR-3665, miR-6836-3p is hsa-miR-6836-3p, miR-6821-5p is hsa-miR-6821-5p, miR-6805-5p is hsa-miR-6805-5p, miR-4728-5p is hsa-miR-4728-5p, miR-6726-5p is hsa-miR-6726-5p, miR-197-5p is hsa-miR-197-5p, miR-149-3p is hsa-miR-149-3p, miR-6850-5p is hsa-miR-6850-5p, miR-4476 is hsa-miR-4476, miR-6858-5p is hsa-miR-6858-5p, miR-564 is hsa-miR-564, miR-4763-3p is hsa-miR-4763-3p, miR-575 is hsa-miR-575, miR-6771-5p is hsa-miR-6771-5p, miR-1231 is hsa-miR-1231, miR-1908-3p is hsa-miR-1908-3p, miR-150-3p is hsa-miR-150-3p, miR-3937 is hsa-miR-3937, miR-887-3p is hsa-miR-887-3p, miR-3940-5p is hsa-miR-3940-5p, miR-4741 is hsa-miR-4741, miR-6808-5p is hsa-miR-6808-5p, miR-6869-5p is hsa-miR-6869-5p, miR-5090 is hsa-miR-5090, miR-615-5p is hsa-miR-615-5p, miR-8072 is hsa-miR-8072, miR-128-1-5p is hsa-miR-128-1-5p, miR-1238-5p is hsa-miR-1238-5p, miR-365a-5p is hsa-miR-365a-5p, miR-204-3p is hsa-miR-204-3p, miR-4492 is hsa-miR-4492, miR-6785-5p is hsa-miR-6785-5p, miR-6511a-5p is hsa-miR-6511a-5p, miR-4525 is hsa-miR-4525, miR-1915-5p is hsa-miR-1915-5p, miR-3180 is hsa-miR-3180, miR-6879-5p is hsa-miR-6879-5p, miR-1199-5p is hsa-miR-1199-5p, miR-6746-5p is hsa-miR-6746-5p, miR-711 is hsa-miR-711, miR-663b is hsa-miR-663b, miR-4707-3p is hsa-miR-4707-3p, miR-6893-5p is hsa-miR-6893-5p, miR-4675 is hsa-miR-4675, miR-4638-5p is hsa-miR-4638-5p, miR-4651 is hsa-miR-4651, miR-6087 is hsa-miR-6087, miR-4665-5p is hsa-miR-4665-5p, miR-4758-5p is hsa-miR-4758-5p, miR-6887-5p is hsa-miR-6887-5p, miR-3620-5p is hsa-miR-3620-5p, miR-1909-3p is hsa-miR-1909-3p, miR-7641 is hsa-miR-7641, miR-6724-5p is hsa-miR-6724-5p, miR-1343-3p is hsa-miR-1343-3p, miR-6780b-5p is hsa-miR-6780b-5p, miR-4484 is hsa-miR-4484, miR-4690-5p is hsa-miR-4690-5p, miR-4429 is hsa-miR-4429, miR-1227-5p is hsa-miR-1227-5p, miR-4725-3p is hsa-miR-4725-3p, miR-6861-5p is hsa-miR-6861-5p, miR-6812-5p is hsa-miR-6812-5p, miR-3197 is hsa-miR-3197, miR-8059 is hsa-miR-8059, miR-3185 is hsa-miR-3185, miR-4706 is hsa-miR-4706, miR-4497 is hsa-miR-4497, miR-3131 is hsa-miR-3131, miR-6806-5p is hsa-miR-6806-5p, miR-187-5p is hsa-miR-187-5p, miR-3180-3p is hsa-miR-3180-3p, miR-6848-5p is hsa-miR-6848-5p, miR-6820-5p is hsa-miR-6820-5p, miR-6800-5p is hsa-miR-6800-5p, miR-6717-5p is hsa-miR-6717-5p, miR-6795-5p is hsa-miR-6795-5p, miR-4632-5p is hsa-miR-4632-5p, miR-665 is hsa-miR-665, miR-6778-5p is hsa-miR-6778-5p, miR-3663-3p is hsa-miR-3663-3p, miR-4689 is hsa-miR-4689, miR-211-3p is hsa-miR-211-3p, miR-6511b-5p is hsa-miR-6511b-5p, miR-4750-5p is hsa-miR-4750-5p, miR-6126 is hsa-miR-6126, miR-614 is hsa-miR-614, miR-7110-5p is hsa-miR-7110-5p, miR-744-5p is hsa-miR-744-5p, miR-6769a-5p is hsa-miR-6769a-5p, miR-4792 is hsa-miR-4792, miR-5787 is hsa-miR-5787, miR-6798-5p is hsa-miR-6798-5p, miR-6781-5p is hsa-miR-6781-5p, miR-4419b is hsa-miR-4419b, miR-4446-3p is hsa-miR-4446-3p, miR-4259 is hsa-miR-4259, miR-5572 is hsa-miR-5572, miR-6075 is hsa-miR-6075, miR-296-3p is hsa-miR-296-3p, miR-6891-5p is hsa-miR-6891-5p, miR-4745-5p is hsa-miR-4745-5p, miR-6775-5p is hsa-miR-6775-5p, miR-6870-5p is hsa-miR-6870-5p, miR-920 is hsa-miR-920, miR-4530 is hsa-miR-4530, miR-6819-5p is hsa-miR-6819-5p, miR-6825-5p is hsa-miR-6825-5p, miR-7847-3p is hsa-miR-7847-3p, miR-6131 is hsa-miR-6131, miR-4433-3p is hsa-miR-4433-3p, miR-1228-5p is hsa-miR-1228-5p, miR-6743-5p is hsa-miR-6743-5p, miR-1268a is hsa-miR-1268a, miR-3917 is hsa-miR-3917, miR-6786-5p is hsa-miR-6786-5p, miR-3154 is hsa-miR-3154, miR-638 is hsa-miR-638, miR-6741-5p is hsa-miR-6741-5p, miR-6889-5p is hsa-miR-6889-5p, miR-6840-3p is hsa-miR-6840-3p, miR-6510-5p is hsa-miR-6510-5p, miR-3188 is hsa-miR-3188, miR-551b-5p is hsa-miR-551b-5p, miR-5001-5p is hsa-miR-5001-5p, miR-1268b is hsa-miR-1268b, miR-7107-5p is hsa-miR-7107-5p, miR-6824-5p is hsa-miR-6824-5p, miR-6732-5p is hsa-miR-6732-5p, miR-371a-5p is hsa-miR-371a-5p, miR-6794-5p is hsa-miR-6794-5p, miR-6779-5p is hsa-miR-6779-5p, miR-4271 is hsa-miR-4271, miR-5195-3p is hsa-miR-5195-3p, miR-6762-5p is hsa-miR-6762-5p, miR-939-5p is hsa-miR-939-5p, miR-1247-3p is hsa-miR-1247-3p, miR-6777-5p is hsa-miR-6777-5p, miR-6722-3p is hsa-miR-6722-3p, miR-3656 is hsa-miR-3656, miR-4688 is hsa-miR-4688, miR-3195 is hsa-miR-3195, miR-6766-5p is hsa-miR-6766-5p, miR-4447 is hsa-miR-4447, miR-4656 is hsa-miR-4656, miR-7108-5p is hsa-miR-7108-5p, miR-3191-3p is hsa-miR-3191-3p, miR-1273g-3p is hsa-miR-1273g-3p, miR-4463 is hsa-miR-4463, miR-2861 is hsa-miR-2861, miR-3196 is hsa-miR-3196, miR-6877-5p is hsa-miR-6877-5p, miR-3679-5p is hsa-miR-3679-5p, miR-4442 is hsa-miR-4442, miR-6789-5p is hsa-miR-6789-5p, miR-6782-5p is hsa-miR-6782-5p, miR-486-3p is hsa-miR-486-3p, miR-6085 is hsa-miR-6085, miR-4746-3p is hsa-miR-4746-3p, miR-619-5p is hsa-miR-619-5p, miR-937-5p is hsa-miR-937-5p, miR-6803-5p is hsa-miR-6803-5p, miR-4298 is hsa-miR-4298, miR-4454 is hsa-miR-4454, miR-4459 is hsa-miR-4459, miR-7150 is hsa-miR-7150, miR-6880-5p is hsa-miR-6880-5p, miR-4449 is hsa-miR-4449, miR-8063 is hsa-miR-8063, miR-4695-5p is hsa-miR-4695-5p, miR-6132 is hsa-miR-6132, miR-6829-5p is hsa-miR-6829-5p, miR-4486 is hsa-miR-4486, miR-6805-3p is hsa-miR-6805-3p, miR-6826-5p is hsa-miR-6826-5p, miR-4508 is hsa-miR-4508, miR-1343-5p is hsa-miR-1343-5p, miR-7114-5p is hsa-miR-7114-5p, miR-3622a-5p is hsa-miR-3622a-5p, miR-6765-5p is hsa-miR-6765-5p, miR-7845-5p is hsa-miR-7845-5p, miR-3960 is hsa-miR-3960, miR-6749-5p is hsa-miR-6749-5p, miR-1260b is hsa-miR-1260b, miR-6799-5p is hsa-miR-6799-5p, miR-4723-5p is hsa-miR-4723-5p, miR-6784-5p is hsa-miR-6784-5p, miR-5100 is hsa-miR-5100, miR-6769b-5p is hsa-miR-6769b-5p, miR-1207-5p is hsa-miR-1207-5p, miR-642a-3p is hsa-miR-642a-3p, miR-4505 is hsa-miR-4505, miR-4270 is hsa-miR-4270, miR-6721-5p is hsa-miR-6721-5p, miR-7111-5p is hsa-miR-7111-5p, miR-6791-5p is hsa-miR-6791-5p, miR-7109-5p is hsa-miR-7109-5p, miR-4258 is hsa-miR-4258, miR-6515-3p is hsa-miR-6515-3p, miR-6851-5p is hsa-miR-6851-5p, miR-6125 is hsa-miR-6125, miR-4749-5p is hsa-miR-4749-5p, miR-4726-5p is hsa-miR-4726-5p, miR-4513 is hsa-miR-4513, miR-6089 is hsa-miR-6089, miR-6816-5p is hsa-miR-6816-5p, miR-4466 is hsa-miR-4466, miR-4488 is hsa-miR-4488, miR-6752-5p is hsa-miR-6752-5p, and miR-4739 is hsa-miR-4739.

In a preferred embodiment of the method of the present invention, specifically, the nucleic acid (specifically, probe(s) or primer(s)) is selected from the group consisting of the following polynucleotides (a) to (e):
(a) a polynucleotide consisting of a nucleotide sequence represented by any of SEQ ID NOs: 1 to 235 and 851 to 856 or a nucleotide sequence derived from the nucleotide sequence by the replacement of u with t, a variant thereof, a derivative thereof, or a fragment thereof comprising 15 or more consecutive nucleotides,
(b) a polynucleotide comprising a nucleotide sequence represented by any of SEQ ID NOs: 1 to 235 and 851 to 856,
(c) a polynucleotide consisting of a nucleotide sequence complementary to a nucleotide sequence represented by any of SEQ ID NOs: 1 to 235 and 851 to 856 or a nucleotide sequence derived from the nucleotide sequence by the replacement of u with t, a variant thereof, a derivative thereof, or a fragment thereof comprising 15 or more consecutive nucleotides,
(d) a polynucleotide comprising a nucleotide sequence complementary to a nucleotide sequence represented by any of SEQ ID NOs: 1 to 235 and 851 to 856 or a nucleotide sequence derived from the nucleotide sequence by the replacement of u with t, and
(e) a polynucleotide hybridizing under stringent conditions to any of the polynucleotides (a) to (d).

The method of the present invention can further employ nucleic acid(s) capable of specifically binding to at least one polynucleotide selected from the group consisting of miR-760, miR-602, miR-423-5p, miR-92a-2-5p, miR-16-5p, miR-451a, miR-135a-3p, miR-486-5p, miR-4257, miR-92b-5p, miR-1915-3p, miR-718, miR-940, miR-296-5p, miR-23b-3p and miR-92a-3p.

As for such a nucleic acid, specifically, miR-760 is hsa-miR-760, miR-602 is hsa-miR-602, miR-423-5p is hsa-miR-423-5p, miR-92a-2-5p is hsa-miR-92a-2-5p, miR-16-5p is hsa-miR-16-5p, miR-451a is hsa-miR-451a, miR-135a-3p is hsa-miR-135a-3p, miR-486-5p is hsa-miR-486-5p, miR-4257 is hsa-miR-4257, miR-92b-5p is hsa-miR-92b-5p, miR-1915-3p is hsa-miR-1915-3p, miR-718 is hsa-miR-718, miR-940 is hsa-miR-940, miR-296-5p is hsa-miR-296-5p, miR-23b-3p is hsa-miR-23b-3p, and miR-92a-3p is hsa-miR-92a-3p.

In a preferred embodiment, such a nucleic acid is specifically selected from the group consisting of the following polynucleotides (f) to (j):
(f) a polynucleotide consisting of a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(g) a polynucleotide comprising a nucleotide sequence represented by any of SEQ ID NOs: 236 to 251,
(h) a polynucleotide consisting of a nucleotide sequence complementary to a nucleotide sequence represented by any of SEQ ID NOs: 236 to 251 or a nucleotide sequence derived from the nucleotide sequence by the replacement of u with t, a variant thereof, a derivative thereof, or a fragment thereof comprising 15 or more consecutive nucleotides,
(i) a polynucleotide comprising a nucleotide sequence complementary to a nucleotide sequence represented by any of SEQ ID NOs: 236 to 251 or a nucleotide sequence derived from the nucleotide sequence by the replacement of u with t, and
(j) a polynucleotide hybridizing under stringent conditions to any of the polynucleotides (f) to (i).

The nucleic acid(s) further used in the method of the present invention can comprise nucleic acid(s) capable of specifically binding to at least one polynucleotide selected from the group consisting of miR-658, miR-6842-5p, miR-6124, miR-6765-3p, miR-7106-5p, miR-4534, miR-92b-3p, miR-3135b, miR-4687-3p, miR-762, miR-3619-3p, miR-4467, miR-557, miR-1237-5p, miR-1908-5p, miR-4286, miR-6885-5p and miR-6763-5p.

Specifically, miR-658 is hsa-miR-658, miR-6842-5p is hsa-miR-6842-5p, miR-6124 is hsa-miR-6124, miR-6765-3p is hsa-miR-6765-3p, miR-7106-5p is hsa-miR-7106-5p, miR-4534is hsa-miR-4534, miR-92b-3p is hsa-miR-92b-3p, miR-3135b is hsa-miR-3135b, miR-4687-3p is hsa-miR-4687-3p, miR-762 is hsa-miR-762, miR-3619-3p is hsa-miR-3619-3p, miR-4467 is hsa-miR-4467, miR-557 is hsa-miR-557, miR-1237-5p is hsa-miR-1237-5p, miR-1908-5p is hsa-miR-1908-5p, miR-4286 is hsa-miR-4286, miR-6885-5p is hsa-miR-6885-5p, and miR-6763-5p is hsa-miR-6763-5p.

Further, in a preferred embodiment, such nucleic acid(s) is/are specifically a polynucleotide selected from the group consisting of the following polynucleotides (k) to (o):
(k) a polynucleotide consisting of a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(l) a polynucleotide comprising a nucleotide sequence represented by any of SEQ ID NOs: 252 to 269,
(m) a polynucleotide consisting of a nucleotide sequence complementary to a nucleotide sequence represented by any of SEQ ID NOs: 252 to 269 or a nucleotide sequence derived from the nucleotide sequence by the replacement of u with t, a variant thereof, a derivative thereof, or a fragment thereof comprising 15 or more consecutive nucleotides,
(n) a polynucleotide comprising a nucleotide sequence complementary to a nucleotide sequence represented by any of SEQ ID NOs: 252 to 269 or a nucleotide sequence derived from the nucleotide sequence by the replacement of u with t, and
(o) a polynucleotide hybridizing under stringent conditions to any of the polynucleotides (k) to (n).

Examples of the sample used in the method of the present invention can include samples prepared from living tissues (preferably breast tissues) or body fluids such as blood, serum, plasma, and urine from the subjects. Specifically, for example, an RNA-containing sample prepared from the tissue, a polynucleotide-containing sample further prepared therefrom, a body fluid such as blood, serum, plasma, or urine, a portion or the whole of a living tissue collected from the subject by biopsy or the like, or a living tissue excised by surgery can be used, and the sample for measurement can be prepared therefrom.

The subject used herein refers to a mammal, for example, a human, a monkey, a mouse or a rat, and is preferably a human.

The steps of the method of the present invention can be changed according to the type of the sample to be assayed.

In the case of using RNA as an analyte, the detection of breast cancer (cells) can comprise, for example, the following steps (a), (b), and (c):
(a) a step of binding RNA prepared from a sample from a subject or complementary polynucleotides (cDNAs) transcribed from the RNA to a polynucleotide(s) in the kit or the device of the present invention;
(b) a step of measuring the sample-derived RNA or the cDNAs synthesized from the RNA, which is/are bound to the polynucleotide(s) by hybridization using the polynucleotide(s) as nucleic acid probe(s) or by quantitative RT-PCR using the polynucleotide(s) as primer(s); and
(c) a step of evaluating the presence or absence of breast cancer (or breast cancer-derived gene expression) on the basis of the measurement results of the step (b).

For example, various hybridization methods can be used for detecting, examining, evaluating, or diagnosing breast cancer (or breast cancer-derived gene expression) in vitro according to the present invention. For example, Northern blot, Southern blot, RT-PCR, DNA chip analysis, in situ hybridization, Northern hybridization, or Southern hybridization can be used as such a hybridization method.

In the case of using the Northern blot, the presence or absence of expression of each gene or the expression level thereof in the RNA can be detected or measured by use of the nucleic acid probe(s) that can be used in the present invention. Specific examples thereof can include a method which comprises labeling the nucleic acid probe (or a complementary strand) with a radioisotope (³²P, ³³P, ³⁵S, etc.), a fluorescent material, or the like, that hybridizes the labeled product with the tissue-derived RNA from a subject, which is transferred to a nylon membrane or the like according to a routine method, and then detecting and measuring a signal derived from the label (radioisotope or fluorescent material) on the formed DNA/RNA duplex using a radiation detector (examples thereof can include BAS-1800 II (Fujifilm Corp.)) or a fluorescence detector (examples thereof can include STORM 865 (GE Healthcare Japan Corp.)).

In the case of using the quantitative RT-PCR, the presence or absence of expression of each gene or the expression level thereof in the RNA can be detected or measured by use of the primer that can be used in the present invention. Specific examples thereof can include a method which comprises; preparing cDNAs from the tissue-derived RNA of a subject according to a routine procedure; hybridizing a pair of primers (consisting of a plus strand and a reverse strand that bind to the cDNA) of the present invention with the cDNA such that the region of each target gene can be amplified with the cDNA as a template; and performing PCR according to a routine method to detect the obtained double-stranded DNA. The method for detecting the double-stranded DNA can include; a method of performing the PCR using the primers labeled in advance with a radioisotope or a fluorescent material; a method of electrophoresing the PCR product on an agarose gel and staining the double-stranded DNA with ethidium bromide or the like for detection; and a method of transferring the produced double-stranded DNA to a nylon membrane or the like according to a routine method and hybridizing the double-stranded DNA to a labeled nucleic acid probe for detection.

In the case of using the nucleic acid array analysis, an RNA chip or a DNA chip in which the nucleic acid probes (single-stranded or double-stranded) are attached to a substrate (solid phase) is used. Regions having the attached nucleic acid probes are referred to as probe spots, and regions having no attached nucleic acid probe are referred to as blank spots. A gene group immobilized on a solid-phase substrate is generally called a nucleic acid chip, a nucleic acid array, a microarray, or the like. The DNA or RNA array includes a DNA or RNA macroarray and a DNA or RNA microarray. The term “chip” used herein includes all of these arrays. 3D-Gene™ Human miRNA Oligo chip (Toray Industries, Inc.) can be used as the DNA chip, though the DNA chip is not limited thereto.

Examples of the measurement using the DNA chip can include, but are not limited to, a method of detecting and measuring a signal derived from the label on the nucleic acid probe using an image detector (examples thereof can include Typhoon 9410 (GE Healthcare) and 3D-Gene™ scanner (Toray Industries, Inc.)).

The “stringent conditions” used herein are, as mentioned above, conditions under which a nucleic acid probe hybridizes to its target sequence to a larger extent (e.g., a measurement value equal to or larger than “(a mean of background measurement values)+(a standard deviation of the background measurement values)×2”) than that for other sequences.

The stringent conditions are defined by hybridization and subsequent washing conditions. Examples of the hybridization conditions include, but not limited to 30° C. to 60° C. for 1 to 24 hours in a solution containing SSC, a surfactant, formamide, dextran sulfate, a blocking agent(s), etc. In this context, 1×SSC is an aqueous solution (pH 7.0) containing 150 mM sodium chloride and 15 mM sodium citrate. The surfactant includes, for example, SDS (sodium dodecyl sulfate), Triton, or Tween. The hybridization conditions more preferably comprise 3-10×SSC and 0.1-1% SDS. Examples of the conditions for the washing, following the hybridization, which is another condition to define the stringent conditions, can include conditions comprising continuous washing at 30° C. in a solution containing 0.5×SSC and 0.1% SDS, at 30° C. in a solution containing 0.2×SSC and 0.1% SDS, and at 30° C. in a 0.05×SSC solution. It is desirable that the complementary strand should maintain its hybridized state with a target plus strand even by the washing under such conditions. Specifically, examples of such a complementary strand can include a strand consisting of a nucleotide sequence in a completely complementary relationship with the nucleotide sequence of the target plus (+) strand, and a strand consisting of a nucleotide sequence having at least 80%, preferably at least 85%, more preferably at least 90% or at least 95%, for example, at least 98% or at least 99% identity to the strand.

Other examples of the “stringent conditions” for the hybridization are described in, for example, Sambrook, J. & Russel, D., Molecular Cloning, A LABORATORY MANUAL, Cold Spring Harbor Laboratory Press, published on Jan. 15, 2001, Vol. 1, 7.42 to 7.45 and Vol. 2, 8.9 to 8.17, and can be used in the present invention.

Examples of the conditions for carrying out PCR using polynucleotide fragments contained in the kit of the present invention as primers include treatment for approximately 15 seconds to 1 minute at 5 to 10° C. plus a Tm value calculated from the sequences of the primers, using a PCR buffer having composition such as 10 mM Tris-HCL (pH 8.3), 50 mM KCL, and 1 to 2 mM MgCl₂. Examples of the method for calculating such a Tm value include Tm value=2×(the number of adenine residues+the number of thymine residues)+4×(the number of guanine residues+the number of cytosine residues).

In the case of using the quantitative RT-PCR, a commercially available kit for measurement specially designed for quantitatively measuring miRNA, such as TaqMan™ MicroRNA Assays (Life Technologies Corp.); LNA™-based MicroRNA PCR (Exiqon); or Ncode™ miRNA qRT-PCT kit (Invitrogen Corp.) may be used.

For the calculation of gene expression levels, statistical analysis described in, for example, Statistical analysis of gene expression microarray data (Speed T., Chapman and Hall/CRC), and A beginner's guide Microarray gene expression data analysis (Causton H. C. et al., Blackwell publishing) can be used in the present invention, though the calculation method is not limited thereto. For example, twice, preferably 3 times, more preferably 6 times the standard deviation of the measurement values of the blank spots are added to the average measurement value of the blank spots on the DNA chip, and probe spots having a signal value equal to or larger than the resulting value can be regarded as detection spots. Alternatively, the average measurement value of the blank spots is regarded as a background and can be subtracted from the measurement values of the probe spots to determine gene expression levels. A missing value for a gene expression level can be excluded from the analyte, preferably replaced with the smallest value of the gene expression level in each DNA chip, or more preferably replaced with a value obtained by subtracting 0.1 from a logarithmic value of the smallest value of the gene expression level. In order to eliminate low-signal genes, only a gene that shows a gene expression level of 2⁶, preferably 2⁸, more preferably 2¹⁰, or larger, in 20% or more, preferably 50% or more, more preferably 80% or more of the number of measurement samples can be selected as the analyte. Examples of the normalization of the gene expression level include, but are not limited to, global normalization and quantile normalization (Bolstad, B. M. et al., 2003, Bioinformatics, Vol. 19, p. 185-193).

The present invention also provides a method comprising measuring target genes or gene expression levels in a sample from a subject using the polynucleotide, the kit, or the device (e.g., chip) for detection of the present invention, or a combination thereof, preparing a discriminant (discriminant function) with gene expression levels in a sample from a breast cancer patient and a sample from a healthy subject as supervising samples, and determining or evaluating the presence and/or absence of the breast cancer-derived genes in the sample.

Specifically, the present invention further provides the method comprising: a first step of measuring in vitro expression levels of target genes in multiple samples known to determine or evaluate the presence and/or absence of the breast cancer-derived genes in the samples, using the polynucleotides, the kit, or the device (e.g., chip) for detection of the present invention, or a combination thereof; a second step of preparing a discriminant with the measurement values of the expression levels of the target genes (target nucleic acid) obtained in the first step as supervising samples; a third step of measuring in vitro expression levels of the target genes in a sample derived from a subject in the same way as in the first step; and a fourth step of assigning the measurement values of the expression levels of the target genes obtained in the third step to the discriminant obtained in the second step, and determining or evaluating the presence and/or absence of the breast cancer-derived gene in the sample on the basis of the results obtained from the discriminant, wherein the target genes can be detected using the polynucleotides or using polynucleotides for detection contained in the kit or the device (e.g., chip). In this context, the discriminant can be prepared by use of Fisher's discriminant analysis, nonlinear discriminant analysis based on Mahalanobis' distance, neural network, Support Vector Machine (SVM), or the like, though the method is not limited thereto.

When a clustering boundary is a straight line or a hyperplane, the linear discriminant analysis is a method for determining the association of a cluster using Formula 1 as a discriminant. In this formula, x represents an explanatory variable, w represents a coefficient of the explanatory variable, and wo represents a constant term.

$$f ( x ) = {w}_{0} + {\sum }_{i = 1}^{n} {w}_{i} {x}_{i} \qquad Formula 1$$

Values obtained from the discriminant are referred to as discriminant scores. The measurement values of a newly offered data set can be assigned as explanatory variables to the discriminant to determine clusters by the signs of the discriminant scores.

The Fisher's discriminant analysis, one type of linear discriminant analysis, is a dimension reduction method for selecting a dimension suitable for classification, and constructs a highly discriminating synthetic variable by focusing on the variance of synthetic variables and minimizing the variance of data having the same label (Venables, W. N. et al., Modern Applied Statistics with S. Fourth edition. Springer., 2002). In the Fisher's discriminant analysis, direction w of projection is determined so as to maximize Formula 2. In this formula, μ represents an average input, ng represents the number of data associated to class g, and μg represents an average input of the data associated to class g. The numerator and the denominator are the interclass variance and the intraclass variance, respectively, when each data is projected in the direction of the vector w. Discriminant coefficient wi is determined by maximizing this ratio (Takafumi Kanamori et al., “Pattern Recognition”, Kyoritsu Shuppan Co., Ltd. (2009); and Richard O. et al., Pattern Classification Second Edition., Wiley-Interscience, 2000).

$$J ( w ) = \frac{{\sum }_{g = 1}^{G} {n}_{g} ( {w}^{T} {μ}_{g} - {w}^{T} μ ) {( {w}^{T} {μ}_{g} - {w}^{T} μ )}^{T}}{{\sum }_{g = 1}^{G} {\sum }_{i : {y}_{i} = g} ( {w}^{T} {x}_{i} - {w}^{T} {μ}_{g} ) ( {w}^{T} {x}_{i} - {w}^{T} {μ}_{g} )} \qquad Formula 2$$

$$subject to μ = {\sum }_{i - 1}^{n} \frac{{x}_{i}}{n} , {μ}_{g} = {\sum }_{i : {u}_{l} - g}^{n} \frac{{x}_{l}}{{n}_{g}}$$

The Mahalanobis' distance is calculated according to Formula 3 in consideration of data correlation and can be used as nonlinear discriminant analysis for determining a cluster to which a data point is associated, based on a short Mahalanobis' distance from the data point to that cluster. In this formula, μ represents a central vector of each cluster, and S−1 represents an inverse matrix of the variance-covariance matrix of the cluster. The central vector is calculated from explanatory variable x, and an average vector, a median value vector, or the like can be used.

D(x,μ)={(x−μ)^{t}S⁻¹(x−μ)}^{1/2} Formula 3

SVM is a discriminant analysis method devised by V. Vapnik (The Nature of Statistical Leaning Theory, Springer, 1995). Particular data points of a data set having known classes are defined as explanatory variables, and classes are defined as objective variables. A boundary plane called hyperplane for correctly classifying the data set into the known classes is determined, and a discriminant for data classification is determined using the boundary plane. Then, the measurement values of a newly offered data set can be assigned as explanatory variables to the discriminant to determine classes. In this respect, the result of the discriminant analysis may be the associated classe, may be a probability of being classified into correct classes, or may be the distance from the hyperplane. In SVM, a method of nonlinearly converting a feature vector to a high dimension and performing linear discriminant analysis in the space is known as a method for tackling nonlinear problems. An expression in which an inner product of two factors in a nonlinearly mapped space is expressed only by inputs in their original spaces is called kernel. Examples of the kernel can include a linear kernel, a RBF (Radial Basis Function) kernel, and a Gaussian kernel. While highly dimensional mapping is performed according to the kernel, the optimum discriminant, i.e., a discriminant, can be actually constructed by mere calculation according to the kernel, which avoids calculating features in the mapped space (e.g., Hideki Aso et al., Frontier of Statistical Science 6 “Statistics of pattern recognition and learning—New concepts and approaches”, Iwanami Shoten, Publishers (2004); Nello Cristianini et al., Introduction to SVM, Kyoritsu Shuppan Co., Ltd. (2008)).

C-support vector classification (C-SVC), one type of SVM, comprises preparing a hyperplane by supervising a data set with the explanatory variables of two groups and classifying an unknown data set into either of the groups (C. Cortes et al., 1995, Machine Learning, Vol. 20, p. 273-297).

Exemplary calculation of the C-SVC discriminant that can be used in the method of the present invention will be given below. First, all subjects are divided into two groups, i.e., a breast cancer patient group and a healthy subject group. For example, breast tissue examination can be used for a reference under which each subject is confirmed either as a breast cancer patient or as a healthy subject.

Next, a data set consisting of comprehensive gene expression levels of serum-derived samples of the two divided groups (hereinafter, this data set is referred to as a training cohort) is prepared, and a C-SVC discriminant is determined by explanatory variables that are genes found to differ clearly in their gene expression levels between the two groups, and objective variables (e.g., −1 and +1) that are the grouping. An optimizing objective function is represented by Formula 4 wherein e represents all input vectors, y represents an objective variable, a represents a Lagrange's undetermined multiplier vector, Q represents a positive definite matrix, and C represents a parameter for adjusting constrained conditions.

$${min}_{a} \frac{1}{2} {a}^{T} Qa - {e}^{T} a \qquad Formula 4$$

$$subject to {y}^{T} a = 0 , 0 \leq {a}_{i} \leq C , i = 1 , ... , l ,$$

Formula 5 is a finally obtained discriminant, and a group to which the data point is associated can be determined on the basis of the sign of a value obtained according to the discriminant. In this formula, x represents a support vector, y represents a label indicating the association of a group, a represents the corresponding coefficient, b represents a constant term, and K represents a kernel function.

$$f ( x ) = sgn ( {\sum }_{i = 1}^{l} {y}_{i} {a}_{i} K ( {x}_{l} , x ) + b ) \qquad Formula 5$$

For example, a RBF kernel defined by Formula 6 can be used as the kernel function. In this formula, x represents a support vector, and y represents a kernel parameter for adjusting the complexity of the hyperplane.

K(xᵢ,xⱼ)=exp(−r∥xᵢ−xⱼ∥²),r<0 Formula 6

In addition, an approach such as neural network, k-nearest neighbor algorithms, decision trees, or logistic regression analysis can be selected as a method for determining or evaluating the presence and/or absence of expression of a breast cancer-derived target gene in a sample derived from a subject, or for evaluating the expression level thereof by comparison with a control derived from a healthy subject.

The method of the present invention can comprise, for example, the following steps (a), (b), and (c):
(a) a step of measuring expression level(s) of target gene(s) in tissues containing breast cancer-derived genes derived from breast cancer patients and/or samples already known to be tissues containing no breast cancer-derived gene(s) derived from healthy subjects, using the polynucleotide(s), the kit, or the device (e.g., DNA chip) for detection according to the present invention;
(b) a step of preparing the discriminants of Formulas 1 to 3, 5, and 6 described above from the measurement values of the expression level measured in the step (a); and
(c) a step of measuring expression level(s) of the target gene(s) in a sample derived from a subject using the polynucleotide(s), the kit, or the device (e.g., DNA chip) for diagnosis (detection) according to the present invention, substituting the obtained measurement valuec into the discriminants prepared in the step (b), and determining or evaluating the presence and/or absence of the breast cancer-derived target gene in the sample, or evaluating the expression levels thereof by comparison with a healthy subject-derived control, on the basis of the obtained results. In this context, in the discriminants of Formulas 1 to 3, 5, and 6, x represents an explanatory variable and includes a value obtained by measuring polynucleotide(s) selected from the polynucleotides described in the Section 2 above, or any fragment thereof. Specifically, the explanatory variable for discriminating a breast cancer patient from a healthy subject according to the present invention is gene expression level(s) selected from, for example, the following expression levels (1) to (3):
(1) gene expression level(s) in the serum of a breast cancer patient or a healthy subject measured by any DNA comprising 15 or more consecutive nucleotides in a nucleotide sequence represented by any of SEQ ID NOs: 1 to 235 and 851 to 856 or a complementary sequence thereof,
(2) gene expression level(s) in the serum of a breast cancer patient or a healthy subject measured by any DNA comprising 15 or more consecutive nucleotides in a nucleotide sequence represented by any of SEQ ID NOs: 236 to 251 or a complementary sequence thereof,
(3) gene expression level(s) in the serum of a breast cancer patient or a healthy subject measured by any DNA comprising 15 or more consecutive nucleotides in a nucleotide sequence represented by any of SEQ ID NOs: 252 to 269 or a complementary sequence thereof.

As described above, for the method for determining or evaluating the presence and/or absence of breast cancer-derived gene(s) in a sample derived from a subject, the preparation of a discriminant requires a discriminant prepared from a training cohort. For enhancing the accuracy of the discriminant, it is necessary for the discriminant to use genes that show clear difference between two groups in the training cohort.

Each gene that is used for an explanatory variable in a discriminant is preferably determined as follows. First, comprehensive gene expression levels of a breast cancer patient group and comprehensive gene expression levels of a healthy subject group, both of which are in a training cohort, are used as a data set, the degree of difference in the expression level of each gene between the two groups is determined through the use of, for example, the P value of t test, which is parametric analysis, or the P value of Mann-Whitney's U test or Wilcoxon test, which is nonparametric analysis.

The gene can be regarded as being statistically significant when the critical rate (significance level) of the P value obtained by the test is smaller than, for example, 5%, 1%, or 0.01%.

In order to correct an increased probability of type I error attributed to the repetition of a test, a method known in the art, for example, Bonferroni or Holm method, can be used for the correction (e.g., Yasushi Nagata et al., “Basics of statistical multiple comparison methods”, Scientist Press Co., Ltd. (2007)). As an example of the Bonferroni correction, for example, the P value obtained by a test is multiplied by the number of repetitions of the test, i.e., the number of genes used in the analysis, and the obtained value can be compared with a desired significance level to suppress a probability of causing type I error in the whole test.

Instead of the statistical test, the absolute value (fold change) of an expression ratio of a median value of each gene expression level between gene expression levels of a breast cancer patient group and gene expression levels of a healthy subject group may be calculated to select a gene that is used for an explanatory variable in a discriminant. Alternatively, ROC curves may be prepared using gene expression levels of a breast cancer patient group and a healthy subject group, and a gene that is used for an explanatory variable in a discriminant can be selected on the basis of an AUROC value.

Next, a discriminant that can be calculated by various methods described above is prepared using any number of genes that show large difference in their gene expression levels determined here. Examples of the method for constructing a discriminant that produces the largest discriminant accuracy include a method of constructing a discriminant in every combination of genes that satisfy the significance level of P value, and a method of repetitively evaluating the genes for use in the construction of a discriminant while increasing the number of genes one by one in a descending order of the difference in gene expression level (Furey T S. et al., 2000, Bioinformatics., Vol. 16, p. 906-14). A gene expression level of another independent breast cancer patient or healthy subject is assigned as an explanatory variable to this discriminant to calculate discriminant results of the group to which this independent breast cancer patient or healthy subject is associated. Specifically, the found gene set for diagnosis and the discriminant constructed using the gene set for diagnosis can be evaluated in an independent sample cohort to find a more universal gene set for diagnosis capable of detecting breast cancer and a more universal method for discriminating breast cancer.

Split-sample method is preferably used for evaluating the performance (generality) of the discriminant. Specifically, a data set is divided into a training cohort and a validation cohort, and gene selection by a statistical test and discriminant construction are performed using the training cohort. Accuracy, sensitivity, and specificity are calculated using results of discriminant analysis in the validation cohort according to the discriminant constructed and a true group to which the validation cohort is associated, to evaluate the discriminant performance of the discriminant. On the other hand, instead of dividing a data set, gene selection by a statistical test and discriminant construction may be performed using all of samples, and accuracy, sensitivity, and specificity can be calculated by the discriminat analysis using a newly prepared cohorts for evaluation of the performance of the discriminant.

The present invention provides polynucleotides for detection and disease diagnosis useful in the diagnosis and treatment of breast cancer, a method for detecting breast cancer using the polynucleotide(s), and a kit and a device for the detection of breast cancer, comprising the polynucleotide(s). Particularly, in order to select gene(s) for diagnosis and prepare a discriminant so as to exhibit accuracy beyond the breast cancer diagnosis method using an existing tumor marker CEA, a gene set for diagnosis and a discriminant for the method of the present invention can be constructed, which exhibit accuracy beyond CEA, for example, by comparing expressed genes in serum from a patient confirmed to be negative using CEA but finally found to have breast cancer by detailed examination such as computed tomography using a contrast medium, with genes expressed in serum from a patient having no breast cancer.

For example, the gene set for diagnosis is set to any combination selected from one or two or more of the polynucleotides based on a nucleotide sequence represented by any of SEQ ID NOs: 1 to 235, and 851 to 856, or a complementary sequence thereof as described above; and optionally one or two or more of the polynucleotides based on a nucleotide sequence represented by any of SEQ ID NOs: 236 to 251, or a complementary sequence thereof; and optionally one or two or more of the polynucleotides based on a nucleotide sequence represented by any of SEQ ID NOs: 252 to 269, or a complementary sequence thereof. Further, a discriminant is constructed using expression levels of the gene set for diagnosis in samples from class I breast cancer patients as a result of tissue diagnosis and samples from class II healthy subjects as a result of tissue diagnosis. As a result, the presence or absence of breast cancer-derived genes in an unknown sample can be determined with 100% accuracy at the maximum by measuring expression levels of the gene set for diagnosis in an unknown sample.

### EXAMPLES

Hereinafter, the present invention will be described further specifically with reference to Examples below. However, the scope of the present invention is not intended to be limited by these Examples.

### Reference Example 1

<Collection of Samples from Breast Cancer Patients and Healthy Subjects>

Sera were collected using VENOJECT II vacuum blood collecting tube VP-AS109K60 (Terumo Corp.) from 100 healthy subjects (92 males and 8 females) and 62 breast cancer patients (20 cases with stage I, 24 cases with stage IIA, 7 cases with stage IIB, 2 cases with stage IIIA, 3 cases with stage IIIB, 1 case with stage IIIC, and 5 cases with stage IV) who were confirmed to have no primary cancer other than breast cancer after acquisition of informed consent, and used as a training cohort. Likewise, Sera were collected using VENOJECT II vacuum blood collecting tube VP-AS109K60 (Terumo Corp.) from 50 healthy subjects (44 males and 6 females) and 31 breast cancer patients (9 cases with stage I, 13 cases with stage IIA, 5 cases with stage IIB, 1 case with stage IIIA, 1 case with stage IIIB, 1 case with stage IIIC, and 1 case with stage IV) who were confirmed to have no primary cancer other than breast cancer after acquisition of informed consent, and used as a validation cohort.

<Extraction of Total RNA>

Total RNA was obtained from 300 μL of the serum sample obtained from each of 243 persons in total of 150 healthy subjects and 93 breast cancer patients included in the training cohort and the validation cohort, using a reagent for RNA extraction in 3D-Gene™ RNA extraction reagent from liquid sample kit (Toray Industries, Inc.) according to the protocol provided by the manufacturer.

<Measurement of Gene Expression Level>

miRNAs in the total RNA obtained from the serum samples of each of 243 persons in total of 150 healthy subjects and 93 breast cancer patients included in the training cohort and the validation cohort were fluorescently labeled using 3D-Gene™ miRNA Labeling kit (Toray Industries, Inc.) according to the protocol (ver 2.20) provided by the manufacturer. The oligo DNA chip used was 3D-Gene™ Human miRNA Oligo chip (Toray Industries, Inc.) with attached probes having sequences complementary to 2,555 miRNAs among the miRNAs registered in miRBase Release 20. Hybridization between the miRNAs in the total RNA and the probes on the DNA chip under stringent conditions and washing following the hybridization were performed according to the protocol provided by the manufacturer. The DNA chip was scanned using 3D-Gene™ scanner (Toray Industries, Inc.) to obtain images. Fluorescence intensity was digitized using 3D-Gene™ Extraction (Toray Industries, Inc.). The digitized fluorescence intensity was converted to a logarithmic value with a base of 2 and used as a gene expression level, from which a blank value was subtracted. A missing value was replaced with a value obtained by subtracting 0.1 from a logarithmic value of the smallest value of the gene expression level in each DNA chip. As a result, the comprehensive gene expression levels of the miRNAs in the sera were obtained for the 93 breast cancer patients and the 150 healthy subjects. Calculation and statistical analysis using the digitized gene expression levels of the miRNAs were carried out using R language 3.0.2 (R Development Core Team (2013). R: A language and environment for statistical computing. R Foundation for Statistical Computing, URL http://www.R-projectorg/) and MASS package 7.3-30 (Venables, W. N. & Ripley, B. D. (2002) Modern Applied Statistics with S. Fourth Edition. Springer, New York. ISBN 0-387-95457-0).

### Reference Example 2

<Collection of Samples from Patients with Cancer Other than Breast Cancer>

Sera were collected using VENOJECT II vacuum blood collecting tube VP-AS109K60 (Terumo Corp.) from each of 33 prostate cancer patients confirmed to have no cancer in other organs after acquisition of informed consent, and used as a training cohort together with the samples of 62 breast cancer patients and 102 healthy subjects of Reference Example 1. Likewise, sera were collected using VENOJECT 11 vacuum blood collecting tube VP-AS109K60 (Terumo Corp.) from each of 19 prostate cancer patients confirmed to have no cancer in other organs after acquisition of informed consent, and used as a validation cohort together with the samples of 31 breast cancer patients confirmed to have no cancer in organs other than the breast and 48 healthy subjects of Reference Example 1. Subsequent operations were conducted in the same way as in Reference Example 1.

### Example 1

<Selection of Gene Marker Using the Training Cohort, and Method for Evaluating Breast Cancer Discriminant Performance of the Single Gene Marker Using the Validation Cohort>

In this Example, a gene marker for discriminating a breast cancer patient from a healthy subject was selected from the training cohort and studied in the validation cohort independent of the training cohort, for a method for evaluating the breast cancer discriminant performance of each selected gene marker alone.

Specifically, first, the miRNA expression levels of the training cohort and the validation cohort obtained in the preceding Reference Examples were combined and normalized by quantile normalization.

Next, genes for diagnosis were selected using the training cohort. Here, in order to acquire diagnostic markers with higher reliability, only genes that show the gene expression level of 2⁶ or higher in 50% or more of the samples in either of the breast cancer patient group in the training cohort or the healthy subject group in the training cohort were selected. In order to further acquire statistically significant genes for discriminating a breast cancer patient group from a healthy subject group, the P value obtained by two-tailed t-test assuming equal variance as to each gene expression level was corrected by the Bonferroni method, and genes that satisfied p<0.01 were acquired as gene markers for use in explanatory variables of a discriminant. The obtained genes are described in Table 2.

In this way, hsa-miR-4783-3p, hsa-miR-4730, hsa-miR-1307-3p, hsa-miR-4634, hsa-miR-663a, hsa-miR-4532, hsa-miR-7704, hsa-miR-3178, hsa-miR-6729-5p, hsa-miR-6090, hsa-miR-4732-5p, hsa-miR-3184-5p, hsa-miR-6727-5p, hsa-miR-6088, hsa-miR-4674, hsa-miR-8073, hsa-miR-4787-5p, hsa-miR-1469, hsa-miR-125a-3p, hsa-miR-1233-5p, hsa-miR-885-3p, hsa-miR-6802-5p, hsa-miR-328-5p, hsa-miR-6787-5p, hsa-miR-8069, hsa-miR-6875-5p, hsa-miR-1246, hsa-miR-4734, hsa-miR-6757-5p, hsa-miR-6756-5p, hsa-miR-3665, hsa-miR-6836-3p, hsa-miR-6821-5p, hsa-miR-6805-5p, hsa-miR-4728-5p, hsa-miR-6726-5p, hsa-miR-197-5p, hsa-miR-149-3p, hsa-miR-6850-5p, hsa-miR-4476, hsa-miR-6858-5p, hsa-miR-564, hsa-miR-4763-3p, hsa-miR-575, hsa-miR-6771-5p, hsa-miR-1231, hsa-miR-1908-3p, hsa-miR-150-3p, hsa-miR-3937, hsa-miR-887-3p, hsa-miR-3940-5p, hsa-miR-4741, hsa-miR-6808-5p, hsa-miR-6869-5p, hsa-miR-5090, hsa-miR-615-5p, hsa-miR-8072, hsa-miR-128-1-5p, hsa-miR-1238-5p, hsa-miR-365a-5p, hsa-miR-204-3p, hsa-miR-4492, hsa-miR-6785-5p, hsa-miR-6511a-5p, hsa-miR-4525, hsa-miR-1915-5p, hsa-miR-3180, hsa-miR-6879-5p, hsa-miR-1199-5p, hsa-miR-6746-5p, hsa-miR-711, hsa-miR-663b, hsa-miR-4707-3p, hsa-miR-6893-5p, hsa-miR-4675, hsa-miR-4638-5p, hsa-miR-4651, hsa-miR-6087, hsa-miR-4665-5p, hsa-miR-4758-5p, hsa-miR-6887-5p, hsa-miR-3620-5p, hsa-miR-1909-3p, hsa-miR-7641, hsa-miR-6724-5p, hsa-miR-1343-3p, hsa-miR-6780b-5p, hsa-miR-4484, hsa-miR-4690-5p, hsa-miR-4429, hsa-miR-1227-5p, hsa-miR-4725-3p, hsa-miR-6861-5p, hsa-miR-6812-5p, hsa-miR-3197, hsa-miR-8059, hsa-miR-3185, hsa-miR-4706, hsa-miR-4497, hsa-miR-3131, hsa-miR-6806-5p, hsa-miR-187-5p, hsa-miR-3180-3p, hsa-miR-6848-5p, hsa-miR-6820-5p, hsa-miR-6800-5p, hsa-miR-6717-5p, hsa-miR-6795-5p, hsa-miR-4632-5p, hsa-miR-665, hsa-miR-6778-5p, hsa-miR-3663-3p, hsa-miR-4689, hsa-miR-211-3p, hsa-miR-6511b-5p, hsa-miR-4750-5p, hsa-miR-6126, hsa-miR-614, hsa-miR-7110-5p, hsa-miR-744-5p, hsa-miR-6769a-5p, hsa-miR-4792, hsa-miR-5787, hsa-miR-6798-5p, hsa-miR-6781-5p, hsa-miR-4419b, hsa-miR-4446-3p, hsa-miR-4259, hsa-miR-5572, hsa-miR-6075, hsa-miR-296-3p, hsa-miR-6891-5p, hsa-miR-4745-5p, hsa-miR-6775-5p, hsa-miR-6870-5p, hsa-miR-920, hsa-miR-4530, hsa-miR-6819-5p, hsa-miR-6825-5p, hsa-miR-7847-3p, hsa-miR-6131, hsa-miR-4433-3p, hsa-miR-1228-5p, hsa-miR-6743-5p, hsa-miR-1268a, hsa-miR-3917, hsa-miR-6786-5p, hsa-miR-3154, hsa-miR-638, hsa-miR-6741-5p, hsa-miR-6889-5p, hsa-miR-6840-3p, hsa-miR-6510-5p, hsa-miR-3188,hsa-miR-551b-5p, hsa-miR-5001-5p, hsa-miR-1268b, hsa-miR-7107-5p, hsa-miR-6824-5p, hsa-miR-6732-5p, hsa-miR-371a-5p, hsa-miR-6794-5p, hsa-miR-6779-5p, hsa-miR-4271, hsa-miR-5195-3p, hsa-miR-6762-5p, hsa-miR-939-5p, hsa-miR-1247-3p, hsa-miR-6777-5p, hsa-miR-6722-3p, hsa-miR-3656, hsa-miR-4688, hsa-miR-3195, hsa-miR-6766-5p, hsa-miR-4447, hsa-miR-4656, hsa-miR-7108-5p, hsa-miR-3191-3p, hsa-miR-1273g-3p, hsa-miR-4463, hsa-miR-2861, hsa-miR-3196, hsa-miR-6877-5p, hsa-miR-3679-5p, hsa-miR-4442, hsa-miR-6789-5p, hsa-miR-6782-5p, hsa-miR-486-3p, hsa-miR-6085, hsa-miR-4746-3p, hsa-miR-619-5p, hsa-miR-937-5p, hsa-miR-6803-5p, hsa-miR-4298, hsa-miR-4454, hsa-miR-4459, hsa-miR-7150, hsa-miR-6880-5p, hsa-miR-4449, hsa-miR-8063, hsa-miR-4695-5p, hsa-miR-6132, hsa-miR-6829-5p, hsa-miR-4486, hsa-miR-6805-3p, hsa-miR-6826-5p, hsa-miR-4508, hsa-miR-1343-5p, hsa-miR-7114-5p, hsa-miR-3622a-5p, hsa-miR-6765-5p, hsa-miR-7845-5p, hsa-miR-3960, hsa-miR-6749-5p, hsa-miR-1260b, hsa-miR-6799-5p, hsa-miR-4723-5p, hsa-miR-6784-5p, hsa-miR-5100, hsa-miR-6769b-5p, hsa-miR-1207-5p, hsa-miR-642a-3p, hsa-miR-4505, hsa-miR-4270, hsa-miR-6721-5p, hsa-miR-7111-5p, hsa-miR-6791-5p, hsa-miR-7109-5p, hsa-miR-4258, hsa-miR-6515-3p, hsa-miR-6851-5p, hsa-miR-6125, hsa-miR-4749-5p, hsa-miR-4726-5p, hsa-miR-4513, hsa-miR-760, hsa-miR-602, hsa-miR-423-5p, hsa-miR-92a-2-5p, hsa-miR-16-5p, hsa-miR-451a, hsa-miR-135a-3p, hsa-miR-486-5p, hsa-miR-4257, hsa-miR-92b-5p, hsa-miR-1915-3p, hsa-miR-718, hsa-miR-940, hsa-miR-296-5p, hsa-miR-23b-3p and hsa-miR-92a-3p genes, and polynucleotides consisting of the nucleotide sequences of SEQ ID NOs: 1 to 251 related thereto were found.

Among them, genes newly found as markers for examining the presence or absence of breast cancer are polynucleotides consisting of the nucleotide sequences represented by SEQ ID NOs: 1 to 235.

A discriminant for determining the presence or absence of breast cancer was further prepared by Fisher's discriminant analysis with the expression levels of these genes as indicators. Specifically, any newly found polynucleotide consisting of a nucleotide sequence represented by any of SEQ ID NOs: 1 to 251 in the training cohort was applied for Formula 2 above to prepare a discriminant. Calculated accuracy, sensitivity, and specificity are shown in Table 3. In this respect, a discriminant coefficient and a constant term are shown in Table 4. Here, all of the polynucleotides consisting of the nucleotide sequences represented by SEQ ID NOs: 1 to 251 were selected as markers capable of determining not only invasive ductal breast cancer (56 cases), which is a main type of breast cancer, but also invasive lobular cancer (3 cases) and unusual metastatic carcinoma with poor prognosis (1 case).

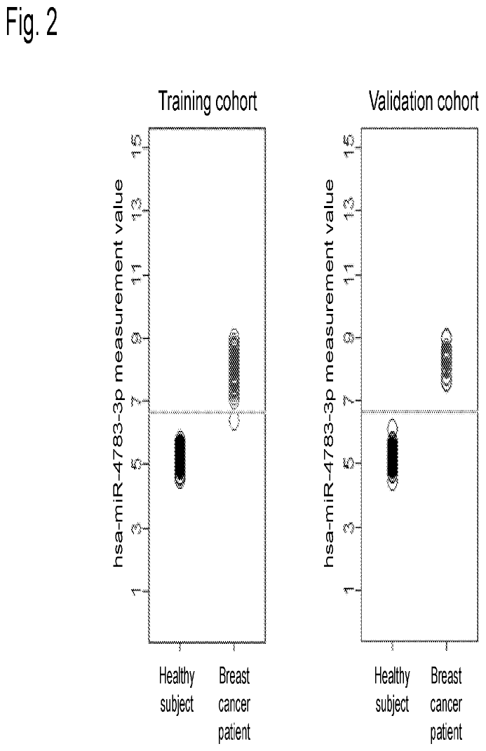

Accuracy, sensitivity, and specificity in the validation cohort were calculated using the discriminant thus prepared, and the discriminant performance of the selected polynucleotides was validated using the independent samples (Table 3). For example, the expression level measurement value of the gene that consists of the nucleotide sequence represented by SEQ ID NO: 1 was compared between the healthy subjects (100 persons) and the breast cancer patients (62 persons) in the training cohort. As a result, the expression level measurement values were found to be significantly lower in the breast cancer patient group than in the healthy subject group (see the left diagram of FIG. 2). These results were also reproducible for the healthy subjects (50 persons) and the breast cancer patients (31 persons) in the validation cohort (see the right diagram of FIG. 2). Likewise, the results obtained about the other polynucleotides shown in SEQ ID NOs: 2 to 251 showed that the expression level measurement values were significantly lower (−) or higher (+) in the breast cancer patient group than in the healthy subject group (Table 2). These results were able to be validated in the validation cohort. For example, as for this nucleotide sequence represented by SEQ ID NO: 1, the number of correctly or incorrectly identified samples in the detection of breast cancer was calculated using the threshold (6.63) that was set in the training cohort and discriminated between the two groups. As a result, 31 true positives, 50 true negatives, 0 false positives, and 0 false negatives were obtained. From these values, 100% accuracy, 100% sensitivity, and 100% specificity were obtained as the detection performance. In this way, the detection performance was calculated as to all of the polynucleotides shown in SEQ ID NOs: 1 to 251, and described in Table 3. Likewise, the polynucleotides consisting of the nucleotide sequences represented by SEQ ID NOs: 2 to 251 shown in Table 2 exhibited sensitivity of 100%, 100%, 90.3%, 96.8%, 100%, 100%, 96.8%, 96.8%, 100%, 100%, 100%, 100%, 96.8%, 96.8%, 100%, 100%, 96.8%, 100%, 93.5%, 96.8%, 96.8%, 100%, 96.8%, 96.8%, 93.5%, 96.8%, 93.5%, 87.1%, 83.9%, 96.8%, 96.8%, 96.8%, 100%, 80.6%, 87.1%, 87.1%, 100%, 90.3%, 90.3%, 90.3%, 100%, 96.8%, 100%, 87.1%, 87.1%, 96.8%, 96.8%, 90.3%, 96.8%, 83.9%, 77.4%, 90%, 83.9%, 96.8%, 93.5%, 80.6%, 96.8%, 90.3%, 93.5%, 90.3%, 87.1%, 87.1%, 96.8%, 83.9%, 87.1%, 77.4%, 90.3%, 77.4%, 90.3%, 83.9%, 74.2%, 93.5%, 87.1%, 93.5%, 93.5%, 77.4%, 90.3%, 87.1%, 87.1%, 83.9%, 87.1%, 93.5%, 77.4%, 93.5%, 74.2%, 83.9%, 100%, 90.3%, 74.2%, 83.9%, 80.6%, 87.1%, 77.4%, 83.9%, 71%, 96.8%, 77.4%, 87.1%, 77.4%, 71%, 90.3%, 80.6%, 67.7%, 77.4%, 87.1%, 74.2%, 83.9%, 77.4%, 71%, 87.1%, 74.2%, 90.3%, 80.6%, 74.2%, 83.9%, 83.9%, 71%, 87.1%, 61.3%, 61.3%, 83.9%, 61.3%, 90.3%, 80.6%, 61.3%, 64.5%, 80.6%, 74.2%, 80.6%, 71%, 71%, 77.4%, 64.5%, 71%, 71%, 83.9%, 74.2%, 83.9%, 63.3%, 64.5%, 71%, 67.7%, 71%, 71%, 74.2%, 71%, 64.5%, 83.9%, 71%, 83.9%, 61.3%, 61.3%, 67.7%, 64.5%, 64.5%, 54.8%, 64.5%, 74.2%, 58.1%, 58.1%, 58.1%, 58.1%, 61.3%, 67.7%, 61.3%, 67.7%, 58.1%, 58.1%, 54.8%, 67.7%, 58.1%, 64.5%, 61.3%, 67.7%, 58.1%, 58.1%, 48.4%, 61.3%, 54.8%, 38.7%, 35.5%, 64.5%, 54.8%, 64.5%, 54.8%, 61.3%, 35.5%, 48.4%, 61.3%, 61.3%, 54.8%, 71%, 61.3%, 45.2%, 48.4%, 29%, 54.8%, 41.9%, 67.7%, 29%, 29%, 53.3%, 51.6%, 45.2%, 35.5%, 41.9%, 41.9%, 48.4%, 41.9%, 35.5%, 41.9%, 35.5%, 48.4%, 32.3%, 41.9%, 41.9%, 41.9%, 35.5%, 35.5%, 41.9%, 61.3%, 32.3%, 45.2%, 38.7%, 51.6%, 29%, 35.5%, 38.7%, 54.8%, 58.1%, 51.6%, 29%, 41.9%, 38.7%, 96.8%, 96.8%, 96.8%, 100%, 96.8%, 87.1%, 80.6%, 100%, 87.1%, 93.5%, 67.7%, 67.7%, 61.3%, 67.7%, 38.7% and 54.8%, respectively, in the validation cohort (Table 3). As seen from Comparative Example mentioned later, the existing marker CEA had sensitivity of 19.4% in the validation cohort (Table 5-2), demonstrating that all of the polynucleotides consisting of the nucleotide sequences represented by SEQ ID Nos: 1 to 251 can discriminate, each alone, breast cancer in the validation cohort with sensitivity beyond CEA.

For example, the 83 polynucleotides consisting of the nucleotide sequences represented by SEQ ID NOs: 1, 2, 3, 4, 6, 7, 8, 9, 10, 11, 12, 13, 14, 15, 16, 17, 19, 20, 21, 22, 23, 24, 25, 26, 27, 28, 31, 32, 33, 34, 35, 36, 38, 40, 42, 44, 45, 47, 48, 49, 50, 55, 59, 64, 65, 66, 68, 70, 73, 75, 78, 79, 80, 81, 84, 88, 92, 93, 95, 97, 98, 99, 102, 109, 113, 119, 122, 124, 128, 130, 133, 145, 149, 169, 236, 237, 238, 239, 240, 241, 242, 243, 244 were able to correctly determine breast cancer in the 9 breast cancer samples of stage 1 contained in the validation cohort. Thus, these polynucleotides can detect even early breast cancer and contributes to the early diagnosis of breast cancer.

### Example 2

<Method a for Evaluating Breast Cancer Discriminant Performance by Combination of Multiple Gene Markers Using the Samples in the Validation Cohort>

In this Example, a method for evaluating breast cancer discriminant performance by a combination of the gene markers selected in Example 1 was studied. Specifically, Fisher's discriminant analysis was conducted as to 31,255 polynucleotide combinations comprising at least one of the newly found polynucleotides consisting of the nucleotide sequences represented by SEQ ID NOs: 1 to 235 among the polynucleotides consisting of the nucleotide sequences represented by SEQ ID NOs: 1 to 251 selected in Example 1, to construct a discriminant for determining the presence or absence of breast cancer. Next, accuracy, sensitivity, and specificity in the validation cohort were calculated using the discriminant thus prepared, and the discriminant performance of the selected polynucleotides was validated using the independent samples.

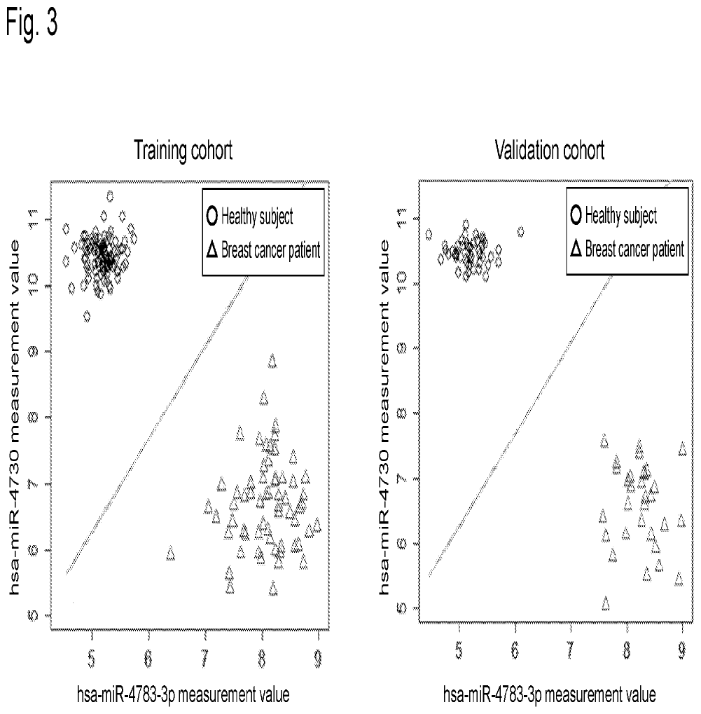

For example, the gene expression level measurement values of the nucleotide sequences represented by SEQ ID NO: 1 and SEQ ID NO: 2 were compared between the healthy subjects and the breast cancer patients. As a result, a scatter diagram that significantly separated the expression level measurement values of the breast cancer patient group from those of the healthy subject group was obtained (see the left diagram of FIG. 3). These results were also reproducible for the healthy subjects (50 persons) and the breast cancer patients (31 persons) in the validation cohort (see the right diagram of FIG. 3). Likewise, a scatter diagram that significantly separated the expression level measurement values of the breast cancer patient group from those of the healthy subject group was also obtained as to the other polynucleotide combinations comprising at least one of the newly found polynucleotides consisting of the nucleotide sequences represented by SEQ ID NOs: 1 to 235 among the polynucleotides consisting of the nucleotide sequences represented by SEQ ID NOs: 1 to 251. These results were able to be validated in the validation cohort. For example, as for these nucleotide sequences represented by SEQ ID NO: 1 and SEQ ID NO: 2, the number of samples that were correctly or incorrectly identified breast cancer was calculated using the discriminant function (0=1.41x+y+0.77) that was set in the training cohort and discriminated between the two groups. As a result, 31 true positives, 50 true negatives, 0 false positives, and 0 false negatives were obtained. From these values, 100% accuracy, 100% sensitivity, and 100% specificity were obtained as the detection performance. In this way, the detection performance was calculated as to all of the polynucleotide combinations comprising at least one of the newly found polynucleotides consisting of the nucleotide sequences represented by SEQ ID NOs: 1 to 235 among the polynucleotides consisting of the nucleotide sequences represented by SEQ ID NOs: 1 to 251. Among them, 250 combinations comprising the expression level measurement value of the polynucleotide consisting of the nucleotide sequence represented by SEQ ID NO: 1 and the detection performance thereof were described in Table 6 as an example. For example, all of the combinations of the expression level measurement values of the polynucleotides consisting of the nucleotide sequences represented by SEQ ID NOs: 1 and 2, SEQ ID NOs: 1 and 3, SEQ ID NOs: 1 and 4, and SEQ ID NOs: 1 and 5 exhibited sensitivity of 100% in the validation cohort. Likewise, all of the combinations of two polynucleotides consisting of the nucleotide sequence represented by SEQ ID NO: 1 and a nucleotide sequence represented by any of SEQ ID NOs: 6 to 251 also exhibited sensitivity of 100%. In addition, the combinations of two polynucleotides consisting of the nucleotide sequences except for SEQ ID NO: 1 were described in Table 7 as an example. As the specific combinations of two polynucleotides, for example, the combinations represented by SEQ ID NOs: 3 and 20, SEQ ID NOs: 6 and 20, SEQ ID NOs: 7 and 20, SEQ ID NOs: 10 and 20, SEQ ID NOs: 20 and 22, SEQ ID NOs: 20 and 238, SEQ ID NOs: 20 and 239, SEQ ID NOs: 12 and 24, SEQ ID NOs: 20 and 24, SEQ ID NOs: 24 and 27, SEQ ID NOs: 24 and 33, SEQ ID NOs: 24 and 236, SEQ ID NOs: 24 and 240, SEQ ID NOs: 3 and 26, SEQ ID NOs: 12 and 26, SEQ ID NOs: 13 and 26, SEQ ID NOs: 17 and 26, SEQ ID NOs: 19 and 26, SEQ ID NOs: 3 and 27, SEQ ID NOs: 5 and 27, SEQ ID NOs: 13 and 27, SEQ ID NOs: 20 and 27, SEQ ID NOs: 26 and 27, SEQ ID NOs: 27 and 120, SEQ ID NOs: 27 and 206, SEQ ID NOs: 27 and 237, SEQ ID NOs: 3 and 30, SEQ ID NOs: 17 and 30, SEQ ID NOs: 27 and 30, SEQ ID NOs: 27 and 33, SEQ ID NOs: 30 and 39, SEQ ID NOs: 30 and 117, SEQ ID NOs: 3 and 33, SEQ ID NOs: 7 and 33, SEQ ID NOs: 10 and 33, SEQ ID NOs: 11 and 33, SEQ ID NOs: 13 and 33, SEQ ID NOs: 25 and 33, SEQ ID NOs: 33 and 244, SEQ ID NOs: 3 and 182, SEQ ID NOs: 6 and 182, SEQ ID NOs: 7 and 182, SEQ ID NOs: 12 and 182, SEQ ID NOs: 27 and 182, SEQ ID NOs: 182 and 236, SEQ ID NOs: 2 and 194, SEQ ID NOs: 7 and 194, SEQ ID NOs: 27 and 194, SEQ ID NOs: 194 and 236, SEQ ID NOs: 2 and 206, SEQ ID NOs: 7 and 206, SEQ ID NOs: 206 and 236, SEQ ID NOs: 2 and 208, SEQ ID NOs: 7 and 208, SEQ ID NOs: 13 and 208, SEQ ID NOs: 20 and 208, and SEQ ID NOs: 27 and 208 exhibited accuracy of 96% or higher for discriminating the breast cancer patients from the healthy subjects in both of the training cohort and the validation cohort. Thus, the combinations of two of the polynucleotides consisting of the nucleotide sequences represented by SEQ ID NOs: 1 to 251 also produced excellent detection sensitivity for breast cancer. Markers for the detection of breast cancer with better sensitivity are obtained by further combining 3, 4, 5, 6, 7, 8, 9, 10 or more of the measurement values of the polynucleotides consisting of the nucleotide sequences represented by SEQ ID NOs: 1 to 251. For example, the newly found polynucleotides consisting of the nucleotide sequences represented by SEQ ID NOs: 1 to 235 among the polynucleotides consisting of the nucleotide sequences represented by SEQ ID NOs: 1 to 251 selected in Example 1, were measured to obtain their expression levels between the healthy subject group and the breast cancer group in the validation cohort. All of the polynucleotides were ranked in the descending order of their P values based on the Student's t-test which indicates statistical significance of a difference between groups (i.e., one having the lowest P value was ranked in the first place), and breast cancer detection sensitivity was evaluated using combinations of one or more polynucleotides to which the polynucleotides were added one by one from the top to bottom according to the rank. In short, the order in which the polynucleotides were combined in this evaluation is in reverse in terms of SEQ ID NOs, such as SEQ ID NO: 235 to SEQ ID NOs: 234, 233, . . . shown in Table 2 in order. As a result, the sensitivity in the validation cohort was 38.7% for 1 polynucleotide (SEQ ID NO: 235), 48.4% for 2 polynucleotides (SEQ ID NOs: 234 and 235), 74.2% for 4 polynucleotides (SEQ ID NOs: 232 to 235), 87.1% for 6 polynucleotides (SEQ ID NOs: 230 to 235), 90.3% for 10 polynucleotides (SEQ ID NOs: 226 to 235), 93.5% for 13 polynucleotides (SEQ ID NOs: 223 to 235), 96.8% for 16 polynucleotides (SEQ ID NOs: 220 to 235), 100% for 20 polynucleotides (SEQ ID NOs: 216 to 235), 100% for 30 polynucleotides (SEQ ID NOs: 206 to 235), 100% for 50 polynucleotides (SEQ ID NOs: 186 to 235), 100% for 100 polynucleotides (SEQ ID NOs: 136 to 235), 100% for 200 polynucleotides (SEQ ID NOs: 36 to 235), and 100% for 235 polynucleotides (SEQ ID NOs: 1 to 235).

These results demonstrated that a combination of multiple polynucleotides can produce higher breast cancer discriminant performance than that of each polynucleotide alone or a combination of a fewer number of polynucleotides. In this context, the combinations of multiple polynucleotides are not limited to the combinations of the polynucleotides added in the order of statistically significant difference as described above, and any combination of multiple polynucleotides can be used in the detection of breast cancer.

From these results, it can be concluded that all of the polynucleotides consisting of the nucleotide sequences represented by SEQ ID NOs: 1 to 251 serve as excellent markers for breast cancer detection.

TABLE 2

| | | | Expression level in |
|---|---|---|---|
| | | P value after | breast cancer |
| | | Bonferroni | patient with respect |
| SEQ ID NO: | Gene name | correction | to healthy subject |
| 1 | hsa-miR-4783-3p | 1.88 · E−98 | + |
| 2 | hsa-miR-4730 | 1.39 · E−95 | − |
| 3 | hsa-miR-1307-3p | 9.90 · E−81 | + |
| 4 | hsa-miR-4634 | 1.73 · E−79 | − |
| 5 | hsa-miR-663a | 2.71 · E−76 | + |
| 6 | hsa-miR-4532 | 2.24 · E−75 | + |
| 7 | hsa-miR-7704 | 4.18 · E−74 | − |
| 8 | hsa-miR-3178 | 2.19 · E−71 | − |
| 9 | hsa-miR-6729-5p | 1.88 · E−69 | − |
| 10 | hsa-miR-6090 | 2.55 · E−68 | + |
| 11 | hsa-miR-4732-5p | 1.36 · E−67 | + |
| 12 | hsa-miR-3184-5p | 8.11 · E−67 | − |
| 13 | hsa-miR-6727-5p | 3.02 · E−64 | − |
| 14 | hsa-miR-6088 | 3.54 · E−64 | + |
| 15 | hsa-miR-4674 | 9.34 · E−64 | − |
| 16 | hsa-miR-8073 | 1.47 · E−63 | + |
| 17 | hsa-miR-4787-5p | 5.85 · E−62 | − |
| 18 | hsa-miR-1469 | 2.97 · E−61 | + |
| 19 | hsa-miR-125a-3p | 9.95 · E−61 | − |
| 20 | hsa-miR-1233-5p | 2.94 · E−60 | + |
| 21 | hsa-miR-885-3p | 6.54 · E−60 | + |
| 22 | hsa-miR-6802-5p | 4.56 · E−59 | + |
| 23 | hsa-miR-328-5p | 1.83 · E−58 | + |
| 24 | hsa-miR-6787-5p | 5.55 · E−58 | + |
| 25 | hsa-miR-8069 | 2.26 · E−57 | − |
| 26 | hsa-miR-6875-5p | 2.72 · E−53 | − |
| 27 | hsa-miR-1246 | 9.57 · E−53 | + |
| 28 | hsa-miR-4734 | 2.06 · E−52 | + |
| 29 | hsa-miR-6757-5p | 4.71 · E−51 | + |
| 30 | hsa-miR-6756-5p | 5.35 · E−51 | + |
| 31 | hsa-miR-3665 | 1.70 · E−50 | − |
| 32 | hsa-miR-6836-3p | 8.38 · E−50 | − |
| 33 | hsa-miR-6821-5p | 1.19 · E−49 | + |
| 34 | hsa-miR-6805-5p | 3.17 · E−49 | − |
| 35 | hsa-miR-4728-5p | 3.69 · E−49 | + |
| 36 | hsa-miR-6726-5p | 5.62 · E−49 | − |
| 37 | hsa-miR-197-5p | 1.43 · E−46 | + |
| 38 | hsa-miR-149-3p | 3.23 · E−46 | + |
| 39 | hsa-miR-6850-5p | 4.58 · E−46 | − |
| 40 | hsa-miR-4476 | 5.54 · E−46 | − |
| 41 | hsa-miR-6858-5p | 9.61 · E−46 | + |
| 42 | hsa-miR-564 | 1.62 · E−44 | − |
| 43 | hsa-miR-4763-3p | 2.01 · E−44 | + |
| 44 | hsa-miR-575 | 3.61 · E−44 | − |
| 45 | hsa-miR-6771-5p | 2.62 · E−43 | − |
| 46 | hsa-miR-1231 | 7.46 · E−43 | − |
| 47 | hsa-miR-1908-3p | 9.60 · E−43 | − |
| 48 | hsa-miR-150-3p | 2.19 · E−42 | − |
| 49 | hsa-miR-3937 | 9.92 · E−42 | − |
| 50 | hsa-miR-887-3p | 2.38 · E−41 | − |
| 51 | hsa-miR-3940-5p | 3.44 · E−41 | − |
| 52 | hsa-miR-4741 | 4.16 · E−41 | − |
| 53 | hsa-miR-6808-5p | 5.42 · E−41 | + |
| 54 | hsa-miR-6869-5p | 1.03 · E−40 | − |
| 55 | hsa-miR-5090 | 5.03 · E−40 | − |
| 56 | hsa-miR-615-5p | 1.94 · E−39 | − |
| 57 | hsa-miR-8072 | 2.71 · E−39 | + |
| 58 | hsa-miR-128-l-5p | 2.72 · E−39 | − |
| 59 | hsa-miR-1238-5p | 7.46 · E−39 | + |
| 60 | hsa-miR-365a-5p | 9.50 · E−39 | + |
| 61 | hsa-miR-204-3p | 1.32 · E−38 | − |
| 62 | hsa-miR-4492 | 3.33 · E−37 | − |
| 63 | hsa-miR-6785-5p | 4.21 · E−37 | − |
| 64 | hsa-miR-6511a-5p | 7.68 · E−37 | + |
| 65 | hsa-miR-4525 | 1.16 · E−36 | − |
| 66 | hsa-miR-1915-5p | 1.34 · E−36 | − |
| 67 | hsa-miR-3180 | 1.07 · E−35 | − |
| 68 | hsa-miR-6879-5p | 1.56 · E−35 | + |
| 69 | hsa-miR-1199-5p | 1.15 · E−34 | − |
| 70 | hsa-miR-6746-5p | 5.65 · E−34 | + |
| 71 | hsa-miR-711 | 5.82 · E−34 | − |
| 72 | hsa-miR-663b | 1.42 · E−33 | − |
| 73 | hsa-miR-4707-3p | 2.19 · E−33 | − |
| 74 | hsa-miR-6893-5p | 6.31 · E−33 | − |
| 75 | hsa-miR-4675 | 6.39 · E−33 | + |
| 76 | hsa-miR-4638-5p | 6.40 · E−33 | − |
| 77 | hsa-miR-4651 | 8.91 · E−33 | − |
| 78 | hsa-miR-6087 | 1.91 · E−32 | + |
| 79 | hsa-miR-4665-5p | 3.57 · E−32 | − |
| 80 | hsa-miR-4758-5p | 4.55 · E−32 | + |
| 81 | hsa-miR-6887-5p | 4.45 · E−31 | + |
| 82 | hsa-miR-3620-5p | 4.64 · E−31 | − |
| 83 | hsa-miR-1909-3p | 5.74 · E−31 | − |
| 84 | hsa-miR-7641 | 8.30 · E−31 | − |
| 85 | hsa-miR-6724-5p | 1.02 · E−30 | + |
| 86 | hsa-miR-1343-3p | 1.19 · E−30 | + |
| 87 | hsa-miR-6780b-5p | 1.22 · E−30 | + |
| 88 | hsa-miR-4484 | 2.77 · E−30 | − |
| 89 | hsa-miR-4690-5p | 3.50 · E−30 | + |
| 90 | hsa-miR-4429 | 2.05 · E−29 | + |
| 91 | hsa-miR-1227-5p | 3.84 · E−29 | + |
| 92 | hsa-miR-4725-3p | 5.39 · E−29 | − |
| 93 | hsa-miR-6861-5p | 5.43 · E−29 | + |
| 94 | hsa-miR-6812-5p | 7.48 · E−29 | + |
| 95 | hsa-miR-3197 | 8.20 · E−29 | + |
| 96 | hsa-miR-8059 | 9.29 · E−29 | + |
| 97 | hsa-miR-3185 | 9.34 · E−29 | + |
| 98 | hsa-miR-4706 | 1.69 · E−28 | + |
| 99 | hsa-miR-4497 | 2.22 · E−28 | − |
| 100 | hsa-miR-3131 | 3.64 · E−28 | + |
| 101 | hsa-miR-6806-5p | 9.04 · E−28 | − |
| 102 | hsa-miR-187-5p | 4.89 · E−27 | − |
| 103 | hsa-miR-3180-3p | 7.10 · E−27 | − |
| 104 | hsa-miR-6848-5p | 7.26 · E−27 | − |
| 105 | hsa-miR-6820-5p | 7.77 · E−27 | − |
| 106 | hsa-miR-6800-5p | 1.80 · E−26 | − |
| 107 | hsa-miR-6717-5p | 1.97 · E−26 | + |
| 108 | hsa-miR-6795-5p | 5.16 · E−26 | + |
| 109 | hsa-miR-4632-5p | 8.43 · E−26 | + |
| 110 | hsa-miR-665 | 2.56 · E−25 | − |
| 111 | hsa-miR-6778-5p | 8.69 · E−25 | − |
| 112 | hsa-miR-3663-3p | 1.09 · E−24 | + |
| 113 | hsa-miR-4689 | 3.36 · E−24 | + |
| 114 | hsa-miR-211-3p | 8.65 · E−24 | + |
| 115 | hsa-miR-6511b-5p | 9.67 · E−24 | + |
| 116 | hsa-miR-4750-5p | 1.07 · E−23 | + |
| 117 | hsa-miR-6126 | 2.06 · E−23 | + |
| 118 | hsa-miR-614 | 2.46 · E−22 | + |
| 119 | hsa-miR-7110-5p | 3.56 · E−22 | − |
| 120 | hsa-miR-744-5p | 5.83 · E−22 | + |
| 121 | hsa-miR-6769a-5p | 1.44 · E−21 | + |
| 122 | hsa-miR-4792 | 2.04 · E−21 | − |
| 123 | hsa-miR-5787 | 3.93 · E−21 | + |
| 124 | hsa-miR-6798-5p | 5.13 · E−21 | − |
| 125 | hsa-miR-6781-5p | 2.43 · E−20 | − |
| 126 | hsa-miR-4419b | 2.59 · E−20 | + |
| 127 | hsa-miR-4446-3p | 7.52 · E−20 | − |
| 128 | hsa-miR-4259 | 8.07 · E−20 | + |
| 129 | hsa-miR-5572 | 1.06 · E−19 | − |
| 130 | hsa-miR-6075 | 1.78 · E−19 | − |
| 131 | hsa-miR-296-3p | 4.75 · E−19 | + |
| 132 | hsa-miR-689l-5p | 8.62 · E−19 | − |
| 133 | hsa-miR-4745-5p | 1.02 · E−18 | − |
| 134 | hsa-miR-6775-5p | 1.17 · E−18 | + |
| 135 | hsa-miR-6870-5p | 1.24 · E−18 | − |
| 136 | hsa-miR-920 | 1.78 · E−18 | + |
| 137 | hsa-miR-4530 | 3.26 · E−18 | − |
| 138 | hsa-miR-6819-5p | 3.67 · E−18 | + |
| 139 | hsa-miR-6825-5p | 5.28 · E−18 | − |
| 140 | hsa-miR-7847-3p | 7.32 · E−18 | + |
| 141 | hsa-miR-6131 | 1.09 · E−17 | + |
| 142 | hsa-miR-4433-3p | 1.35 · E−17 | + |
| 143 | hsa-miR-1228-5p | 9.82 · E−17 | − |
| 144 | hsa-miR-6743-5p | 1.40 · E−16 | + |
| 145 | hsa-miR-1268a | 2.20 · E−16 | − |
| 146 | hsa-miR-3917 | 1.73 · E−15 | + |
| 147 | hsa-miR-6786-5p | 1.89 · E−15 | − |
| 148 | hsa-miR-3154 | 2.12 · E−15 | + |
| 149 | hsa-miR-638 | 2.19 · E−15 | − |
| 150 | hsa-miR-6741-5p | 5.44 · E−15 | + |
| 151 | hsa-miR-6889-5p | 9.29 · E−15 | − |
| 152 | hsa-miR-6840-3p | 1.61 · E−14 | − |
| 153 | hsa-miR-6510-5p | 3.23 · E−14 | − |
| 154 | hsa-miR-3188 | 4.44 · E−14 | + |
| 155 | hsa-miR-551b-5p | 1.71 · E−13 | + |
| 156 | hsa-miR-5001-5p | 2.07 · E−13 | + |
| 157 | hsa-miR-1268b | 2.24 · E−13 | − |
| 158 | hsa-miR-7107-5p | 2.31 · E−13 | − |
| 159 | hsa-miR-6824-5p | 3.30 · E−13 | + |
| 160 | hsa-miR-6732-5p | 3.62 · E−13 | − |
| 161 | hsa-miR-371a-5p | 9.05 · E−13 | + |
| 162 | hsa-miR-6794-5p | 9.74 · E−13 | + |
| 163 | hsa-miR-6779-5p | 1.37 · E−12 | − |
| 164 | hsa-miR-4271 | 1.69 · E−12 | − |
| 165 | hsa-miR-5195-3p | 1.79 · E−12 | + |
| 166 | hsa-miR-6762-5p | 3.61 · E−12 | + |
| 167 | hsa-miR-939-5p | 4.78 · E−12 | − |
| 168 | hsa-miR-1247-3p | 7.37 · E−12 | + |
| 169 | hsa-miR-6777-5p | 9.79 · E−12 | + |
| 170 | hsa-miR-6722-3p | 1.21 · E−11 | + |
| 171 | hsa-miR-3656 | 1.27 · E−11 | + |
| 172 | hsa-miR-4688 | 1.86 · E−11 | + |
| 173 | hsa-miR-3195 | 2.02 · E−11 | − |
| 174 | hsa-miR-6766-5p | 6.97 · E−11 | + |
| 175 | hsa-miR-4447 | 1.08 · E−10 | + |
| 176 | hsa-miR-4656 | 1.12 · E−10 | − |
| 177 | hsa-miR-7108-5p | 1.51 · E−10 | − |
| 178 | hsa-miR-3191-3p | 2.67 · E−10 | + |
| 179 | hsa-miR-1273g-3p | 2.89 · E−10 | − |
| 180 | hsa-miR-4463 | 4.62 · E−10 | + |
| 181 | hsa-miR-2861 | 4.97 · E−10 | + |
| 182 | hsa-miR-3196 | 5.22 · E−10 | − |
| 183 | hsa-miR-6877-5p | 6.47 · E−10 | − |
| 184 | hsa-miR-3679-5p | 1.33 · E−09 | + |
| 185 | hsa-miR-4442 | 1.56 · E−09 | − |
| 186 | hsa-miR-6789-5p | 1.93 · E−09 | − |
| 187 | hsa-miR-6782-5p | 1.97 · E−09 | + |
| 188 | hsa-miR-486-3p | 2.12 · E−09 | − |
| 189 | hsa-miR-6085 | 4.04 · E−09 | + |
| 190 | hsa-miR-4746-3p | 8.57 · E−09 | − |
| 191 | hsa-miR-619-5p | 1.13 · E−08 | − |
| 192 | hsa-miR-937-5p | 1.65 · E−08 | + |
| 193 | hsa-miR-6803-5p | 2.32 · E−08 | + |
| 194 | hsa-miR-4298 | 2.33 · E−08 | + |
| 195 | hsa-miR-4454 | 2.63 · E−08 | + |
| 196 | hsa-miR-4459 | 1.83 · E−07 | + |
| 197 | hsa-miR-7150 | 2.60 · E−07 | + |
| 198 | hsa-miR-6880-5p | 8.86 · E−07 | − |
| 199 | hsa-miR-4449 | 9.44 · E−07 | − |
| 200 | hsa-miR-8063 | 1.05 · E−06 | + |
| 201 | hsa-miR-4695-5p | 1.65 · E−06 | + |
| 202 | hsa-miR-6132 | 1.93 · E−06 | + |
| 203 | hsa-miR-6829-5p | 2.66 · E−06 | + |
| 204 | hsa-miR-4486 | 2.83 · E−06 | − |
| 205 | hsa-miR-6805-3p | 3.24 · E−06 | − |
| 206 | hsa-miR-6826-5p | 4.59 · E−06 | + |
| 207 | hsa-miR-4508 | 6.28 · E−06 | + |
| 208 | hsa-miR-1343-5p | 1.11 · E−05 | − |
| 209 | hsa-miR-7114-5p | 1.35 · E−05 | + |
| 210 | hsa-miR-3622a-5p | 1.53 · E−05 | + |
| 211 | hsa-miR-6765-5p | 1.77 · E−05 | − |
| 212 | hsa-miR-7845-5p | 2.11 · E−05 | − |
| 213 | hsa-miR-3960 | 2.70 · E−05 | − |
| 214 | hsa-miR-6749-5p | 4.51 · E−05 | − |
| 215 | hsa-miR-1260b | 4.83 · E−05 | + |
| 216 | hsa-miR-6799-5p | 5.38 · E−05 | + |
| 217 | hsa-miR-4723-5p | 6.54 · E−05 | + |
| 218 | hsa-miR-6784-5p | 7.88 · E−05 | − |
| 219 | hsa-miR-5100 | 8.28 · E−05 | + |
| 220 | hsa-miR-6769b-5p | 9.25 · E−05 | + |
| 221 | hsa-miR-1207-5p | 1.25 · E−04 | + |
| 222 | hsa-miR-642a-3p | 1.38 · E−04 | − |
| 223 | hsa-miR-4505 | 1.49 · E−04 | + |
| 224 | hsa-miR-4270 | 1.79 · E−04 | − |
| 225 | hsa-miR-6721-5p | 3.50 · E−04 | − |
| 226 | hsa-miR-7111-5p | 5.29 · E−04 | − |
| 227 | hsa-miR-6791-5p | 8.34 · E−04 | + |
| 228 | hsa-miR-7109-5p | 1.07 · E−03 | + |
| 229 | hsa-miR-4258 | 1.55 · E−03 | + |
| 230 | hsa-miR-6515-3p | 2.00 · E−03 | + |
| 231 | hsa-miR-685l-5p | 2.15 · E−03 | − |
| 232 | hsa-miR-6125 | 2.94 · E−03 | − |
| 233 | hsa-miR-4749-5p | 3.39 · E−03 | + |
| 234 | hsa-miR-4726-5p | 6.77 · E−03 | + |
| 235 | hsa-miR-4513 | 9.77 · E−03 | + |
| 236 | hsa-miR-760 | 5.40 · E−76 | − |
| 237 | hsa-miR-602 | 3.27 · E−58 | − |
| 238 | hsa-miR-423-5p | 4.16 · E−57 | − |
| 239 | hsa-miR-92a-2-5p | 7.76 · E−55 | − |
| 240 | hsa-miR-16-5p | 7.58 · E−47 | − |
| 241 | hsa-miR-451a | 1.13 · E−36 | − |
| 242 | hsa-miR-135a-3p | 1.83 · E−35 | − |
| 243 | hsa-miR-486-5p | 8.56 · E−34 | − |
| 244 | hsa-miR-4257 | 4.39 · E−31 | + |
| 245 | hsa-miR-92b-5p | 1.67 · E−30 | + |
| 246 | hsa-miR-1915-3p | 3.95 · E−18 | − |
| 247 | hsa-miR-718 | 1.38 · E−15 | − |
| 248 | hsa-miR-940 | 4.63 · E−15 | + |
| 249 | hsa-miR-296-5p | 2.67 · E−10 | + |
| 250 | hsa-miR-23b-3p | 2.43 · E−03 | + |
| 251 | hsa-miR-92a-3p | 3.86 · E−03 | - |

TABLE 3

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 1 | 99.4 | 98.4 | 100 | 100 | 100 | 100 |
| 2 | 99.4 | 98.4 | 100 | 100 | 100 | 100 |
| 3 | 99.4 | 98.4 | 100 | 100 | 100 | 100 |
| 4 | 99.4 | 98.4 | 100 | 96.3 | 90.3 | 100 |
| 5 | 98.8 | 100 | 98 | 98.8 | 96.8 | 100 |
| 6 | 99.4 | 98.4 | 100 | 100 | 100 | 100 |
| 7 | 100 | 100 | 100 | 100 | 100 | 100 |
| 8 | 99.4 | 98.4 | 100 | 98.8 | 96.8 | 100 |
| 9 | 96.9 | 91.9 | 100 | 98.8 | 96.8 | 100 |
| 10 | 98.8 | 96.8 | 100 | 97.5 | 100 | 96 |
| 11 | 99.4 | 98.4 | 100 | 97.5 | 100 | 96 |
| 12 | 100 | 100 | 100 | 100 | 100 | 100 |
| 13 | 99.4 | 98.4 | 100 | 100 | 100 | 100 |
| 14 | 98.1 | 96.8 | 99 | 98.8 | 96.8 | 100 |
| 15 | 96.3 | 90.3 | 100 | 98.8 | 96.8 | 100 |
| 16 | 98.1 | 95.2 | 100 | 98.8 | 100 | 98 |
| 17 | 98.8 | 96.8 | 100 | 100 | 100 | 100 |
| 18 | 98.8 | 96.8 | 100 | 96.3 | 96.8 | 96 |
| 19 | 99.4 | 100 | 99 | 100 | 100 | 100 |
| 20 | 98.8 | 98.4 | 99 | 97.5 | 93.5 | 100 |
| 21 | 98.1 | 98.4 | 98 | 96.3 | 96.8 | 96 |
| 22 | 98.1 | 95.2 | 100 | 98.8 | 96.8 | 100 |
| 23 | 96.9 | 93.5 | 99 | 100 | 100 | 100 |
| 24 | 97.5 | 93.5 | 100 | 98.8 | 96.8 | 100 |
| 25 | 97.5 | 93.5 | 100 | 96.3 | 96.8 | 96 |
| 26 | 96.3 | 90.3 | 100 | 96.3 | 93.5 | 98 |
| 27 | 96.9 | 93.5 | 99 | 96.3 | 96.8 | 96 |
| 28 | 95.7 | 88.7 | 100 | 97.5 | 93.5 | 100 |
| 29 | 96.9 | 95.2 | 98 | 95.1 | 87.1 | 100 |
| 30 | 95.1 | 87.1 | 100 | 91.4 | 83.9 | 96 |
| 31 | 96.9 | 91.9 | 100 | 98.8 | 96.8 | 100 |
| 32 | 95.1 | 88.7 | 99 | 98.8 | 96.8 | 100 |
| 33 | 94.4 | 96.8 | 93 | 98.8 | 96.8 | 100 |
| 34 | 96.9 | 91.9 | 100 | 100 | 100 | 100 |
| 35 | 96.9 | 95.2 | 98 | 88.9 | 80.6 | 94 |
| 36 | 95.1 | 88.7 | 99 | 95.1 | 87.1 | 100 |
| 37 | 96.3 | 91.9 | 99 | 95.1 | 87.1 | 100 |
| 38 | 96.3 | 93.5 | 98 | 100 | 100 | 100 |
| 39 | 95.7 | 91.9 | 98 | 95.1 | 90.3 | 98 |
| 40 | 96.9 | 95.2 | 98 | 95.1 | 90.3 | 98 |
| 41 | 94.4 | 88.7 | 98 | 93.8 | 90.3 | 96 |
| 42 | 97.5 | 98.4 | 97 | 98.8 | 100 | 98 |
| 43 | 98.1 | 98.4 | 98 | 97.5 | 96.8 | 98 |
| 44 | 97.5 | 93.5 | 100 | 100 | 100 | 100 |
| 45 | 94.4 | 88.7 | 98 | 95.1 | 87.1 | 100 |
| 46 | 96.9 | 91.9 | 100 | 93.8 | 87.1 | 98 |
| 47 | 95.7 | 88.7 | 100 | 98.8 | 96.8 | 100 |
| 48 | 96.9 | 95.2 | 98 | 92.6 | 96.8 | 90 |
| 49 | 95.7 | 93.5 | 97 | 93.8 | 90.3 | 96 |
| 50 | 95.7 | 96.8 | 95 | 97.5 | 96.8 | 98 |
| 51 | 92.6 | 83.9 | 98 | 92.6 | 83.9 | 98 |
| 52 | 93.8 | 93.5 | 94 | 90.1 | 77.4 | 98 |
| 53 | 93.8 | 90.3 | 96 | 91.2 | 90 | 92 |
| 54 | 92.6 | 80.6 | 100 | 93.8 | 83.9 | 100 |
| 55 | 95.7 | 91.8 | 98 | 98.8 | 96.8 | 100 |
| 56 | 97.5 | 95.2 | 99 | 96.3 | 93.5 | 98 |
| 57 | 93.8 | 88.7 | 97 | 86.4 | 80.6 | 90 |
| 58 | 97.5 | 96.8 | 98 | 96.3 | 96.8 | 96 |
| 59 | 95.1 | 90.3 | 98 | 96.3 | 90.3 | 100 |
| 60 | 95.1 | 88.7 | 99 | 95.1 | 93.5 | 96 |
| 61 | 93.8 | 90.3 | 96 | 84 | 90.3 | 80 |
| 62 | 91.3 | 80.3 | 98 | 95.1 | 87.1 | 100 |
| 63 | 93.2 | 95.2 | 92 | 88.9 | 87.1 | 90 |
| 64 | 92.6 | 85.5 | 97 | 98.8 | 96.8 | 100 |
| 65 | 93.2 | 87.1 | 97 | 93.8 | 83.9 | 100 |
| 66 | 93.8 | 87.1 | 98 | 92.6 | 87.1 | 96 |
| 67 | 87.7 | 79 | 93 | 88.9 | 77.4 | 96 |
| 68 | 92.6 | 83.9 | 98 | 96.3 | 90.3 | 100 |
| 69 | 91.4 | 82.3 | 97 | 90.1 | 77.4 | 98 |
| 70 | 93.2 | 87.1 | 97 | 91.4 | 90.3 | 92 |
| 71 | 92.6 | 87.1 | 96 | 93.8 | 83.9 | 100 |
| 72 | 93.8 | 83.9 | 100 | 90.1 | 74.2 | 100 |
| 73 | 95.7 | 91.9 | 98 | 97.5 | 93.5 | 100 |
| 74 | 92.6 | 90.3 | 94 | 87.7 | 87.1 | 88 |
| 75 | 90.1 | 79 | 97 | 96.3 | 93.5 | 98 |
| 76 | 93.8 | 91.9 | 95 | 93.8 | 93.5 | 94 |
| 77 | 93.8 | 87.1 | 98 | 90.1 | 77.4 | 98 |
| 78 | 92.6 | 82.3 | 99 | 91.4 | 90.3 | 92 |
| 79 | 90.7 | 80.6 | 97 | 90.1 | 87.1 | 92 |
| 80 | 90.7 | 75.8 | 100 | 95.1 | 87.1 | 100 |
| 81 | 91.4 | 85.5 | 95 | 87.7 | 83.9 | 90 |
| 82 | 90.7 | 88.7 | 92 | 91.4 | 87.1 | 94 |
| 83 | 93.2 | 83.9 | 99 | 96.3 | 93.5 | 98 |
| 84 | 89.5 | 90.3 | 89 | 84 | 77.4 | 88 |
| 85 | 90.1 | 82.3 | 95 | 93.8 | 93.5 | 94 |
| 86 | 92.6 | 83.9 | 98 | 87.7 | 74.2 | 96 |
| 87 | 88.9 | 79 | 95 | 93.8 | 83.9 | 100 |
| 88 | 92 | 88.7 | 94 | 92.6 | 100 | 88 |
| 89 | 90.7 | 85.5 | 94 | 91.4 | 90.3 | 92 |
| 90 | 92.6 | 90.3 | 94 | 90.1 | 74.2 | 100 |
| 91 | 89.5 | 74.2 | 99 | 92.6 | 83.9 | 98 |
| 92 | 89.5 | 82.3 | 94 | 90.1 | 80.6 | 96 |
| 93 | 89.5 | 79 | 96 | 90.1 | 87.1 | 92 |
| 94 | 89.5 | 80.6 | 95 | 84 | 77.4 | 88 |
| 95 | 89.5 | 91.9 | 88 | 86.4 | 83.9 | 88 |
| 96 | 87.7 | 79 | 93 | 88.9 | 71 | 100 |
| 97 | 90.1 | 88.7 | 91 | 91.4 | 96.8 | 88 |
| 98 | 90.1 | 83.9 | 94 | 86.4 | 77.4 | 92 |
| 99 | 87.6 | 83.6 | 90 | 82.7 | 87.1 | 80 |
| 100 | 88.3 | 80.6 | 93 | 88.9 | 77.4 | 96 |
| 101 | 93.2 | 85.5 | 98 | 88.9 | 71 | 100 |
| 102 | 88.3 | 74.2 | 97 | 95.1 | 90.3 | 98 |
| 103 | 87.7 | 80.6 | 92 | 90.1 | 80.6 | 96 |
| 104 | 87.7 | 77.4 | 94 | 80.2 | 67.7 | 88 |
| 105 | 92 | 88.7 | 94 | 82.7 | 77.4 | 86 |
| 106 | 86.4 | 77.4 | 92 | 93.8 | 87.1 | 98 |
| 107 | 89.5 | 85.5 | 92 | 86.4 | 74.2 | 94 |
| 108 | 88.9 | 80.6 | 94 | 81.5 | 83.9 | 80 |
| 109 | 91.4 | 87.1 | 94 | 87.7 | 77.4 | 94 |
| 110 | 88.3 | 75.8 | 96 | 86.4 | 71 | 96 |
| ill | 86.4 | 82.3 | 89 | 90.1 | 87.1 | 92 |
| 112 | 86.4 | 67.7 | 98 | 86.4 | 74.2 | 94 |
| 113 | 85.8 | 74.2 | 93 | 95.1 | 90.3 | 98 |
| 114 | 95.1 | 88.7 | 99 | 92.6 | 80.6 | 100 |
| 115 | 90.1 | 82.3 | 95 | 87.7 | 74.2 | 96 |
| 116 | 90.1 | 82.3 | 95 | 88.9 | 83.9 | 92 |
| 117 | 83.3 | 72.6 | 90 | 88.9 | 83.9 | 92 |
| 118 | 86.4 | 83.9 | 88 | 80.2 | 71 | 86 |
| 119 | 86.4 | 80.6 | 90 | 88.9 | 87.1 | 90 |
| 120 | 88.3 | 79 | 94 | 84 | 61.3 | 98 |
| 121 | 84.6 | 72.6 | 92 | 75.3 | 61.3 | 84 |
| 122 | 84.6 | 74.2 | 91 | 85.2 | 83.9 | 86 |
| 123 | 82.7 | 79 | 85 | 77.8 | 61.3 | 88 |
| 124 | 84.6 | 80.6 | 87 | 85.2 | 90.3 | 82 |
| 125 | 92 | 82.3 | 98 | 91.4 | 80.6 | 98 |
| 126 | 82.7 | 69.4 | 91 | 84 | 61.3 | 98 |
| 127 | 88.9 | 72.6 | 99 | 86.4 | 64.5 | 100 |
| 128 | 88.9 | 83.9 | 92 | 91.4 | 80.6 | 98 |
| 129 | 87 | 82.3 | 90 | 84 | 74.2 | 90 |
| 130 | 84 | 66.1 | 95 | 86.4 | 80.6 | 90 |
| 131 | 79.6 | 69.4 | 86 | 80.2 | 71 | 86 |
| 132 | 84.6 | 71 | 93 | 84 | 71 | 92 |
| 133 | 81.5 | 71 | 88 | 85.2 | 77.4 | 90 |
| 134 | 81.5 | 61.3 | 94 | 81.5 | 64.5 | 92 |
| 135 | 82.1 | 66.1 | 92 | 82.7 | 71 | 90 |
| 136 | 83.3 | 74.2 | 89 | 82.7 | 71 | 90 |
| 137 | 83.3 | 79 | 86 | 77.8 | 83.9 | 74 |
| 138 | 80.2 | 67.7 | 88 | 79 | 74.2 | 82 |
| 139 | 84 | 74.2 | 90 | 86.4 | 83.9 | 88 |
| 140 | 81.5 | 71 | 88 | 85 | 63.3 | 98 |
| 141 | 82.1 | 67.7 | 91 | 82.7 | 64.5 | 94 |
| 142 | 79.6 | 69.4 | 86 | 80.2 | 71 | 86 |
| 143 | 83.3 | 75.8 | 88 | 76.5 | 67.7 | 82 |
| 144 | 87.7 | 74.2 | 96 | 85.2 | 71 | 94 |
| 145 | 83.3 | 79 | 86 | 80.2 | 71 | 86 |
| 146 | 81.5 | 69.4 | 89 | 81.5 | 74.2 | 86 |
| 147 | 84.6 | 69.4 | 94 | 84 | 71 | 92 |
| 148 | 85.8 | 72.6 | 94 | 77.8 | 64.5 | 86 |
| 149 | 85.2 | 66.1 | 97 | 93.8 | 83.9 | 100 |
| 150 | 79.6 | 62.9 | 90 | 80.2 | 71 | 86 |
| 151 | 80.9 | 67.7 | 89 | 86.4 | 83.9 | 88 |
| 152 | 82.7 | 67.7 | 92 | 79 | 61.3 | 90 |
| 153 | 83.3 | 67.7 | 93 | 79 | 61.3 | 90 |
| 154 | 79.6 | 64.5 | 89 | 84 | 67.7 | 94 |
| 155 | 79.6 | 66.1 | 88 | 82.7 | 64.5 | 94 |
| 156 | 78.4 | 59.7 | 90 | 81.5 | 64.5 | 92 |
| 157 | 77.2 | 66.1 | 84 | 71.6 | 54.8 | 82 |
| 158 | 77.8 | 61.3 | 88 | 79 | 64.5 | 88 |
| 159 | 84 | 67.7 | 94 | 85.2 | 74.2 | 92 |
| 160 | 80.2 | 69.4 | 87 | 71.6 | 58.1 | 80 |
| 161 | 77.2 | 56.5 | 90 | 76.5 | 58.1 | 88 |
| 162 | 79 | 61.3 | 90 | 70.4 | 58.1 | 78 |
| 163 | 79.6 | 64.5 | 89 | 81.5 | 58.1 | 96 |
| 164 | 82.1 | 62.9 | 94 | 84 | 61.3 | 98 |
| 165 | 77.8 | 59.7 | 89 | 82.7 | 67.7 | 92 |
| 166 | 82.1 | 56.5 | 98 | 82.7 | 61.3 | 96 |
| 167 | 77.8 | 69.4 | 83 | 79 | 67.7 | 86 |
| 168 | 72.8 | 56.5 | 83 | 70.4 | 58.1 | 78 |
| 169 | 80.2 | 64.5 | 90 | 72.8 | 58.1 | 82 |
| 170 | 80.2 | 53.2 | 97 | 79 | 54.8 | 94 |
| 171 | 83.3 | 74.2 | 89 | 81.5 | 67.7 | 90 |
| 172 | 79 | 69.4 | 85 | 74.1 | 58.1 | 84 |
| 173 | 79.6 | 54.8 | 95 | 84 | 64.5 | 96 |
| 174 | 77.2 | 56.5 | 90 | 80.2 | 61.3 | 92 |
| 175 | 77.2 | 54.8 | 91 | 76.5 | 67.7 | 82 |
| 176 | 83.3 | 72.6 | 90 | 79 | 58.1 | 92 |
| 177 | 77.2 | 59.7 | 88 | 77.8 | 58.1 | 90 |
| 178 | 77.8 | 59.7 | 89 | 77.8 | 48.4 | 96 |
| 179 | 78.4 | 56.5 | 92 | 80.2 | 61.3 | 92 |
| 180 | 72.2 | 45.2 | 89 | 80.2 | 54.8 | 96 |
| 181 | 74.7 | 50 | 90 | 70.4 | 38.7 | 90 |
| 182 | 75.9 | 59.7 | 86 | 61.7 | 35.5 | 78 |
| 183 | 77.2 | 61.3 | 87 | 76.5 | 64.5 | 84 |
| 184 | 75.9 | 58.1 | 87 | 77.8 | 54.8 | 92 |
| 185 | 74.7 | 59.7 | 84 | 75.3 | 64.5 | 82 |
| 186 | 77.8 | 54.8 | 92 | 77.8 | 54.8 | 92 |
| 187 | 77.2 | 64.5 | 85 | 70.4 | 61.3 | 76 |
| 188 | 78.4 | 54.8 | 93 | 74.1 | 35.5 | 98 |
| 189 | 72.8 | 61.3 | 80 | 72.8 | 48.4 | 88 |
| 190 | 77.8 | 54.8 | 92 | 75.3 | 61.3 | 84 |
| 191 | 83.3 | 62.9 | 96 | 81.5 | 61.3 | 94 |
| 192 | 74.7 | 50 | 90 | 80.2 | 54.8 | 96 |
| 193 | 69.8 | 43.5 | 86 | 74.1 | 71 | 76 |
| 194 | 74.7 | 53.2 | 88 | 81.5 | 61.3 | 94 |
| 195 | 75.3 | 54.8 | 88 | 69.1 | 45.2 | 84 |
| 196 | 74.7 | 56.5 | 86 | 75.3 | 48.4 | 92 |
| 197 | 74.1 | 54.8 | 86 | 69.1 | 29 | 94 |
| 198 | 69.8 | 48.4 | 83 | 72.8 | 54.8 | 84 |
| 199 | 74.1 | 50 | 89 | 72.8 | 41.9 | 92 |
| 200 | 83.3 | 59.7 | 98 | 87.7 | 67.7 | 100 |
| 201 | 68.5 | 41.9 | 85 | 65.4 | 29 | 88 |
| 202 | 75.3 | 45.2 | 94 | 71.6 | 29 | 98 |
| 203 | 71.6 | 38.7 | 92 | 75 | 53.3 | 88 |
| 204 | 71.6 | 53.2 | 83 | 67.9 | 51.6 | 78 |
| 205 | 72.2 | 54.8 | 83 | 69.1 | 45.2 | 84 |
| 206 | 70.4 | 45.2 | 86 | 67.9 | 35.5 | 88 |
| 207 | 72.8 | 48.4 | 88 | 65.4 | 41.9 | 80 |
| 208 | 69.1 | 45.2 | 84 | 67.9 | 41.9 | 84 |
| 209 | 69.1 | 41.9 | 86 | 72.8 | 48.4 | 88 |
| 210 | 74.7 | 51.6 | 89 | 72.8 | 41.9 | 92 |
| 211 | 71 | 53.2 | 82 | 61.7 | 35.5 | 78 |
| 212 | 71 | 45.2 | 87 | 61.7 | 41.9 | 74 |
| 213 | 71 | 24.2 | 100 | 75.3 | 35.5 | 100 |
| 214 | 71 | 45.2 | 87 | 76.5 | 48.4 | 94 |
| 215 | 75.3 | 53.2 | 89 | 65.4 | 32.3 | 86 |
| 216 | 71.6 | 50 | 85 | 71.6 | 41.9 | 90 |
| 217 | 69.8 | 43.5 | 86 | 67.9 | 41.9 | 84 |
| 218 | 71 | 40.3 | 90 | 71.6 | 41.9 | 90 |
| 219 | 71 | 40.3 | 90 | 70.4 | 35.5 | 92 |
| 220 | 73.5 | 45.2 | 91 | 66.7 | 35.5 | 86 |
| 221 | 71.6 | 43.5 | 89 | 72.8 | 41.9 | 92 |
| 222 | 70.4 | 46.8 | 85 | 76.5 | 61.3 | 86 |
| 223 | 77.8 | 46.8 | 97 | 72.8 | 32.3 | 98 |
| 224 | 70.4 | 43.5 | 87 | 72.8 | 45.2 | 90 |
| 225 | 67.3 | 37.1 | 86 | 74.1 | 38.7 | 96 |
| 226 | 67.9 | 37.1 | 87 | 75.3 | 51.6 | 90 |
| 227 | 71 | 41.9 | 89 | 66.7 | 29 | 90 |
| 228 | 69.1 | 40.3 | 87 | 65.4 | 35.5 | 84 |
| 229 | 71.6 | 46.8 | 87 | 65.4 | 38.7 | 82 |
| 230 | 69.1 | 40.3 | 87 | 71.6 | 54.8 | 82 |
| 231 | 71 | 41.9 | 89 | 72.8 | 58.1 | 82 |
| 232 | 71.6 | 41.9 | 90 | 76.5 | 51.6 | 92 |
| 233 | 75.3 | 38.7 | 98 | 72.8 | 29 | 100 |
| 234 | 66 | 33.9 | 86 | 64.2 | 41.9 | 78 |
| 235 | 69.8 | 37.1 | 90 | 67.9 | 38.7 | 86 |
| 236 | 100 | 100 | 100 | 98.8 | 96.8 | 100 |
| 237 | 96.9 | 93.5 | 99 | 98.8 | 96.8 | 100 |
| 238 | 98.8 | 98.4 | 99 | 98.8 | 96.8 | 100 |
| 239 | 96.9 | 95.2 | 98 | 98.8 | 100 | 98 |
| 240 | 95.1 | 93.5 | 96 | 96.3 | 96.8 | 96 |
| 241 | 95 | 91.8 | 97 | 91.4 | 87.1 | 94 |
| 242 | 90.7 | 85.5 | 94 | 91.4 | 80.6 | 98 |
| 243 | 91.4 | 85.5 | 95 | 96.3 | 100 | 94 |
| 244 | 88.9 | 80.6 | 94 | 90.1 | 87.1 | 92 |
| 245 | 94.4 | 87.1 | 99 | 95.1 | 93.5 | 96 |
| 246 | 83.3 | 69.4 | 92 | 81.5 | 67.7 | 90 |
| 247 | 83.3 | 61.3 | 97 | 84 | 67.7 | 94 |
| 248 | 79.6 | 64.5 | 89 | 81.5 | 61.3 | 94 |
| 249 | 75.9 | 56.5 | 88 | 77.8 | 67.7 | 84 |
| 250 | 72.2 | 41.9 | 91 | 70.4 | 38.7 | 90 |
| 251 | 69.1 | 41.9 | 86 | 77.8 | 54.8 | 92 |

TABLE 4

| | Discriminant | Constant |
|---|---|---|
| SEQ ID NO: | coefficient | term |
| 1 | 2.857 | 18.934 |
| 2 | 2.104 | 18.009 |
| 3 | 2.003 | 12.711 |
| 4 | 3.992 | 34.175 |
| 5 | 3.801 | 39.484 |
| 6 | 2.447 | 30.974 |
| 7 | 4.663 | 59.811 |
| 8 | 5.763 | 63.889 |
| 9 | 5.239 | 59.962 |
| 10 | 6.627 | 84.995 |
| 11 | 1.955 | 14.548 |
| 12 | 2.330 | 14.910 |
| 13 | 4.150 | 48.634 |
| 14 | 3.013 | 32.040 |
| 15 | 2.055 | 18.255 |
| 16 | 2.774 | 19.077 |
| 17 | 5.084 | 64.746 |
| 18 | 4.563 | 46.280 |
| 19 | 1.821 | 9.652 |
| 20 | 2.266 | 26.872 |
| 21 | 2.626 | 14.611 |
| 22 | 3.963 | 34.565 |
| 23 | 4.250 | 47.336 |
| 24 | 3.079 | 27.833 |
| 25 | 6.783 | 80.858 |
| 26 | 3.237 | 25.431 |
| 27 | 1.634 | 14.286 |
| 28 | 2.807 | 33.656 |
| 29 | 2.658 | 20.910 |
| 30 | 4.607 | 39.043 |
| 31 | 5.299 | 67.817 |
| 32 | 2.652 | 19.984 |
| 33 | 4.337 | 38.009 |
| 34 | 4.682 | 48.862 |
| 35 | 5.352 | 38.578 |
| 36 | 2.977 | 26.855 |
| 37 | 2.882 | 21.114 |
| 38 | 5.933 | 55.485 |
| 39 | 5.346 | 55.986 |
| 40 | 1.915 | 11.901 |
| 41 | 4.597 | 34.155 |
| 42 | 2.048 | 10.706 |
| 43 | 4.172 | 34.713 |
| 44 | 1.763 | 9.076 |
| 45 | 5.446 | 45.658 |
| 46 | 2.725 | 15.338 |
| 47 | 1.886 | 10.786 |
| 48 | 2.535 | 15.378 |
| 49 | 4.415 | 34.502 |
| 50 | 2.583 | 15.891 |
| 51 | 3.927 | 44.058 |
| 52 | 3.643 | 32.462 |
| 53 | 6.086 | 41.540 |
| 54 | 3.018 | 38.864 |
| 55 | 3.179 | 22.917 |
| 56 | 2.338 | 13.833 |
| 57 | 5.682 | 67.831 |
| 58 | 2.838 | 18.281 |
| 59 | 2.954 | 19.780 |
| 60 | 3.642 | 21.511 |
| 61 | 1.972 | 23.626 |
| 62 | 4.194 | 40.279 |
| 63 | 3.430 | 28.886 |
| 64 | 3.095 | 19.571 |
| 65 | 2.739 | 17.283 |
| 66 | 1.356 | 7.292 |
| 67 | 4.404 | 35.065 |
| 68 | 3.358 | 27.546 |
| 69 | 2.497 | 15.102 |
| 70 | 3.754 | 25.584 |
| 71 | 3.860 | 28.692 |
| 72 | 2.543 | 20.396 |
| 73 | 2.485 | 13.944 |
| 74 | 2.870 | 22.658 |
| 75 | 2.136 | 17.183 |
| 76 | 2.226 | 12.375 |
| 77 | 4.610 | 47.394 |
| 78 | 3.174 | 36.978 |
| 79 | 2.980 | 26.297 |
| 80 | 3.396 | 29.485 |
| 81 | 3.446 | 23.243 |
| 82 | 4.082 | 29.368 |
| 83 | 3.358 | 28.190 |
| 84 | 1.537 | 10.210 |
| 85 | 4.715 | 45.652 |
| 86 | 2.213 | 17.839 |
| 87 | 3.088 | 27.043 |
| 88 | 2.399 | 24.629 |
| 89 | 4.002 | 24.314 |
| 90 | 2.863 | 17.406 |
| 91 | 4.805 | 44.782 |
| 92 | 3.372 | 29.988 |
| 93 | 4.132 | 30.521 |
| 94 | 3.851 | 23.002 |
| 95 | 3.959 | 36.256 |
| 96 | 2.941 | 23.102 |
| 97 | 3.006 | 20.900 |
| 98 | 3.728 | 29.308 |
| 99 | 2.595 | 30.882 |
| 100 | 2.755 | 20.339 |
| 101 | 3.144 | 19.162 |
| 102 | 1.952 | 17.956 |
| 103 | 4.489 | 37.034 |
| 104 | 4.734 | 32.179 |
| 105 | 3.519 | 24.011 |
| 106 | 4.202 | 32.975 |
| 107 | 2.654 | 17.634 |
| 108 | 4.562 | 28.615 |
| 109 | 4.900 | 37.966 |
| 110 | 2.427 | 15.293 |
| 111 | 2.332 | 16.477 |
| 112 | 2.960 | 35.679 |
| 113 | 2.931 | 27.885 |
| 114 | 2.409 | 15.430 |
| 115 | 2.659 | 15.558 |
| 116 | 2.722 | 15.656 |
| 117 | 3.039 | 32.084 |
| 118 | 2.066 | 14.991 |
| 119 | 2.114 | 14.125 |
| 120 | 2.812 | 19.320 |
| 121 | 4.273 | 28.046 |
| 122 | 1.987 | 11.296 |
| 123 | 4.888 | 61.938 |
| 124 | 3.116 | 29.551 |
| 125 | 3.831 | 36.923 |
| 126 | 3.114 | 20.097 |
| 127 | 1.704 | 11.244 |
| 128 | 2.708 | 16.078 |
| 129 | 2.902 | 17.111 |
| 130 | 3.522 | 27.388 |
| 131 | 2.509 | 16.024 |
| 132 | 3.797 | 26.247 |
| 133 | 2.381 | 26.930 |
| 134 | 5.446 | 44.750 |
| 135 | 2.830 | 18.893 |
| 136 | 2.243 | 13.704 |
| 137 | 2.742 | 24.223 |
| 138 | 5.572 | 41.312 |
| 139 | 2.328 | 13.203 |
| 140 | 4.269 | 27.904 |
| 141 | 1.790 | 19.364 |
| 142 | 3.970 | 28.366 |
| 143 | 3.831 | 42.123 |
| 144 | 2.470 | 23.361 |
| 145 | 3.643 | 37.749 |
| 146 | 3.590 | 21.954 |
| 147 | 6.096 | 72.341 |
| 148 | 4.450 | 26.391 |
| 149 | 2.450 | 28.929 |
| 150 | 3.765 | 26.311 |
| 151 | 3.107 | 20.676 |
| 152 | 3.191 | 26.734 |
| 153 | 2.247 | 12.949 |
| 154 | 3.520 | 21.019 |
| 155 | 1.939 | 10.899 |
| 156 | 3.714 | 28.667 |
| 157 | 3.379 | 30.812 |
| 158 | 3.869 | 28.692 |
| 159 | 4.714 | 30.302 |
| 160 | 3.800 | 29.719 |
| 161 | 3.111 | 23.178 |
| 162 | 4.700 | 38.234 |
| 163 | 5.628 | 38.292 |
| 164 | 2.984 | 23.210 |
| 165 | 2.953 | 20.742 |
| 166 | 2.411 | 18.314 |
| 167 | 2.915 | 19.822 |
| 168 | 4.774 | 29.161 |
| 169 | 3.435 | 22.933 |
| 170 | 4.014 | 33.267 |
| 171 | 4.788 | 52.969 |
| 172 | 4.261 | 30.393 |
| 173 | 2.698 | 20.419 |
| 174 | 3.739 | 22.190 |
| 175 | 3.142 | 19.164 |
| 176 | 3.424 | 23.274 |
| 177 | 4.230 | 35.956 |
| 178 | 3.270 | 18.860 |
| 179 | 2.287 | 16.040 |
| 180 | 3.760 | 39.967 |
| 181 | 5.991 | 71.910 |
| 182 | 6.044 | 68.999 |
| 183 | 4.325 | 29.700 |
| 184 | 3.000 | 19.877 |
| 185 | 3.823 | 34.710 |
| 186 | 3.882 | 35.815 |
| 187 | 3.870 | 23.356 |
| 188 | 2.281 | 17.158 |
| 189 | 5.818 | 59.167 |
| 190 | 2.555 | 15.132 |
| 191 | 1.200 | 8.000 |
| 192 | 4.027 | 33.373 |
| 193 | 6.532 | 69.738 |
| 194 | 3.836 | 23.124 |
| 195 | 2.198 | 25.446 |
| 196 | 3.531 | 27.965 |
| 197 | 3.619 | 27.108 |
| 198 | 2.671 | 18.647 |
| 199 | 3.253 | 19.608 |
| 200 | 1.449 | 12.340 |
| 201 | 4.621 | 33.285 |
| 202 | 3.174 | 24.106 |
| 203 | 3.280 | 19.895 |
| 204 | 3.339 | 22.048 |
| 205 | 2.930 | 20.016 |
| 206 | 2.705 | 17.147 |
| 207 | 5.111 | 63.738 |
| 208 | 4.111 | 39.848 |
| 209 | 4.418 | 30.103 |
| 210 | 3.749 | 22.673 |
| 211 | 4.949 | 48.983 |
| 212 | 3.159 | 19.444 |
| 213 | 3.351 | 49.006 |
| 214 | 4.185 | 40.143 |
| 215 | 2.257 | 19.312 |
| 216 | 4.261 | 34.145 |
| 217 | 2.984 | 26.230 |
| 218 | 3.064 | 35.877 |
| 219 | 2.311 | 23.637 |
| 220 | 4.200 | 26.644 |
| 221 | 3.404 | 21.751 |
| 222 | 3.076 | 22.488 |
| 223 | 3.273 | 26.709 |
| 224 | 5.275 | 40.615 |
| 225 | 3.461 | 24.300 |
| 226 | 5.789 | 41.289 |
| 227 | 4.602 | 40.407 |
| 228 | 5.697 | 41.534 |
| 229 | 2.323 | 20.843 |
| 230 | 3.839 | 25.014 |
| 231 | 3.506 | 21.544 |
| 232 | 4.082 | 45.878 |
| 233 | 3.909 | 30.033 |
| 234 | 4.028 | 26.498 |
| 235 | 4.173 | 25.119 |
| 236 | 3.887 | 30.454 |
| 237 | 2.014 | 10.053 |
| 238 | 2.607 | 16.589 |
| 239 | 1.925 | 14.853 |
| 240 | 1.301 | 6.443 |
| 241 | 1.152 | 8.583 |
| 242 | 1.774 | 11.079 |
| 243 | 2.154 | 13.220 |
| 244 | 3.309 | 23.530 |
| 245 | 3.226 | 25.503 |
| 246 | 3.387 | 34.563 |
| 247 | 2.652 | 16.404 |
| 248 | 3.534 | 22.326 |
| 249 | 3.779 | 28.687 |
| 250 | 1.490 | 9.528 |
| 251 | 2.072 | 13.756 |

TABLE 5-1
Training cohort

| Sample name | Cancer stage | CEA(ng/mL) |
|---|---|---|
| BB001 | IV | 1(−) |
| BB006 | IIIB | 14.4(+) |
| BB007 | IIA | 2.6(−) |
| BB009 | IIIC | 4.9(−) |
| BB010 | IIA | 1.2(−) |
| BB012 | I | 2.7(−) |
| BB013 | I | 14(−) |
| BB014 | IIA | 13.5(+) |
| BB015 | I | 3.5(−) |
| BB017 | IIA | 1.9(−) |
| BB018 | IIA | 2.1(−) |
| BB019 | I | 1.2(−) |
| BB023 | IIA | 2.8(−) |
| BB024 | I | 1.5(−) |
| BB026 | I | 3.8(−) |
| BB027 | IIB | 3.4(−) |
| BB028 | IV | 7.4(+) |
| BB032 | I | 1.7(−) |
| BB033 | IIB | 2.6(−) |
| BB035 | IIA | 2.7(−) |
| BB037 | IIB | 1.2(−) |
| BB038 | IV | 0.8(−) |
| BB039 | IIIA | 4.7(−) |
| BB041 | IIA | 2.1(−) |
| BB042 | IIIB | 1.3(−) |
| BB043 | IIIA | 1.2(−) |
| BB046 | I | 1.7(−) |
| BB047 | I | 1(−) |
| BB048 | I | 1.9(−) |
| BB049 | IIA | 0.6(−) |
| BB051 | IIA | 1(−) |
| BB052 | I | 2.4(−) |
| BB054 | IIA | 1.3(−) |
| BB056 | IIA | 1.9(−) |
| BB057 | IIIB | 3460(+) |
| BB058 | IIA | 0.8(−) |
| BB060 | I | 0.5(−) |
| BB061 | I | 1.4(−) |
| BB064 | IV | 2.4(−) |
| BB065 | IIB | 6.6(+) |
| BB068 | IIA | 0.6(−) |
| BB070 | IIB | 0.7(−) |
| BB071 | IIB | 1.8(−) |
| BB073 | I | 1.1(−) |
| BB076 | I | 2(−) |
| BB077 | IIA | 2.9(−) |
| BB078 | IIA | 2.3(−) |
| BB079 | IIA | 1.1(−) |
| BB082 | I | 1.5(−) |
| BB084 | IV | 435(+) |
| BB085 | IIA | 3.1(−) |
| BB086 | IIA | 1.5(−) |
| BB087 | IIA | 1.7(−) |
| BB089 | I | 2(−) |
| BB092 | I | 3.6(−) |
| BB093 | IIB | 3.7(−) |
| BB094 | I | 6(+) |
| BB095 | IIA | 2.4(−) |
| BB096 | I | 4.6(−) |
| BB097 | IIA | 1.8(−) |
| BB098 | IIA | 2.6(−) |
| BB099 | IIA | 4.2(−) |
| | Sensitivity (%) | 11.3 |

TABLE 5-2
Validation cohort

| Sample name | Cancer stage | CEA(ng/mL) |
|---|---|---|
| BB002 | IIIC | 1.3(−) |
| BB005 | IIA | 0.6(−) |
| BB008 | IIA | 0.7(−) |
| BB011 | IIB | 1.4(−) |
| BB016 | I | 2.9(−) |
| BB020 | IIB | 1.5(−) |
| BB021 | IIB | 1.5(−) |
| BB022 | IIA | 0.6(−) |
| BB025 | IIA | 4.1(−) |
| BB029 | IIIA | 2.7(−) |
| BB030 | IIA | 12.7(+) |
| BB031 | IIA | 1.4(−) |
| BB034 | IIB | 4.2(−) |
| BB036 | I | 3.5(−) |
| BB040 | IIB | 1.7(−) |
| BB050 | IIIB | 11.1(+) |
| BB053 | I | 0.8(−) |
| BB055 | IIA | 2.8(−) |
| BB059 | IIA | 10.2(+) |
| BB062 | IIA | 5.9(+) |
| BB063 | I | 0.7(−) |
| BB069 | IIA | 0.7(−) |
| BB072 | IIA | 1.1(−) |
| BB074 | IIA | 6.4(+) |
| BB075 | I | 0.5(−) |
| BB080 | IV | 6.6(+) |
| BB081 | I | 0.8(−) |
| BB083 | I | 0.7(−) |
| BB088 | I | 1(−) |
| BB090 | I | 5.4(+) |
| BB091 | IIA | 1.2(−) |
| | Sensitivity (%) | 19.4 |

For CEA, 5 ng/ml or lower was indicated by as “−”, while values exceedingthese were indicated as “+”

TABLE 6

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 12 | 100 | 100 | 100 | 100 | 100 | 100 |
| 13 | 99.4 | 98.4 | 100 | 100 | 100 | 100 |
| 14 | 100 | 100 | 100 | 100 | 100 | 100 |
| 15 | 99.4 | 98.4 | 100 | 100 | 100 | 100 |
| 16 | 100 | 100 | 100 | 100 | 100 | 100 |
| 17 | 100 | 100 | 100 | 100 | 100 | 100 |
| 18 | 100 | 100 | 100 | 100 | 100 | 100 |
| 19 | 99.4 | 98.4 | 100 | 100 | 100 | 100 |
| 110 | 100 | 100 | 100 | 100 | 100 | 100 |
| 111 | 100 | 100 | 100 | 100 | 100 | 100 |
| 112 | 100 | 100 | 100 | 100 | 100 | 100 |
| 113 | 100 | 100 | 100 | 100 | 100 | 100 |
| 114 | 100 | 100 | 100 | 100 | 100 | 100 |
| 115 | 100 | 100 | 100 | 100 | 100 | 100 |
| 116 | 100 | 100 | 100 | 100 | 100 | 100 |
| 117 | 100 | 100 | 100 | 100 | 100 | 100 |
| 118 | 99.4 | 98.4 | 100 | 100 | 100 | 100 |
| 119 | 100 | 100 | 100 | 100 | 100 | 100 |
| 120 | 100 | 100 | 100 | 100 | 100 | 100 |
| 121 | 99.4 | 98.4 | 100 | 100 | 100 | 100 |
| 122 | 100 | 100 | 100 | 100 | 100 | 100 |
| 123 | 100 | 100 | 100 | 100 | 100 | 100 |
| 124 | 100 | 100 | 100 | 100 | 100 | 100 |
| 125 | 100 | 100 | 100 | 98.8 | 100 | 98 |
| 126 | 100 | 100 | 100 | 100 | 100 | 100 |
| 127 | 100 | 100 | 100 | 100 | 100 | 100 |
| 128 | 99.4 | 98.4 | 100 | 100 | 100 | 100 |
| 129 | 100 | 100 | 100 | 100 | 100 | 100 |
| 130 | 100 | 100 | 100 | 100 | 100 | 100 |
| 131 | 100 | 100 | 100 | 100 | 100 | 100 |
| 132 | 100 | 100 | 100 | 100 | 100 | 100 |
| 133 | 99.4 | 98.4 | 100 | 100 | 100 | 100 |
| 134 | 100 | 100 | 100 | 100 | 100 | 100 |
| 135 | 100 | 100 | 100 | 100 | 100 | 100 |
| 136 | 100 | 100 | 100 | 100 | 100 | 100 |
| 137 | 100 | 100 | 100 | 100 | 100 | 100 |
| 138 | 99.4 | 98.4 | 100 | 100 | 100 | 100 |
| 139 | 100 | 100 | 100 | 100 | 100 | 100 |
| 140 | 100 | 100 | 100 | 100 | 100 | 100 |
| 141 | 100 | 100 | 100 | 100 | 100 | 100 |
| 142 | 100 | 100 | 100 | 100 | 100 | 100 |
| 143 | 99.4 | 98.4 | 100 | 100 | 100 | 100 |
| 144 | 100 | 100 | 100 | 100 | 100 | 100 |
| 145 | 100 | 100 | 100 | 100 | 100 | 100 |
| 146 | 99.4 | 98.4 | 100 | 100 | 100 | 100 |
| 147 | 100 | 100 | 100 | 100 | 100 | 100 |
| 148 | 99.4 | 98.4 | 100 | 100 | 100 | 100 |
| 149 | 99.4 | 98.4 | 100 | 100 | 100 | 100 |
| 150 | 99.4 | 98.4 | 100 | 100 | 100 | 100 |
| 151 | 100 | 100 | 100 | 100 | 100 | 100 |
| 152 | 100 | 100 | 100 | 100 | 100 | 100 |
| 153 | 99.4 | 98.4 | 100 | 100 | 100 | 100 |
| 154 | 99.4 | 98.4 | 100 | 100 | 100 | 100 |
| 155 | 100 | 100 | 100 | 100 | 100 | 100 |
| 156 | 99.4 | 98.4 | 100 | 100 | 100 | 100 |
| 157 | 100 | 100 | 100 | 100 | 100 | 100 |
| 158 | 100 | 100 | 100 | 100 | 100 | 100 |
| 159 | 100 | 100 | 100 | 100 | 100 | 100 |
| 160 | 99.4 | 98.4 | 100 | 100 | 100 | 100 |
| 161 | 99.4 | 98.4 | 100 | 100 | 100 | 100 |
| 162 | 100 | 100 | 100 | 100 | 100 | 100 |
| 163 | 99.4 | 98.4 | 100 | 100 | 100 | 100 |
| 164 | 100 | 100 | 100 | 100 | 100 | 100 |
| 165 | 99.4 | 98.4 | 100 | 100 | 100 | 100 |
| 166 | 99.4 | 98.4 | 100 | 100 | 100 | 100 |
| 167 | 100 | 100 | 100 | 100 | 100 | 100 |
| 168 | 100 | 100 | 100 | 100 | 100 | 100 |
| 169 | 100 | 100 | 100 | 100 | 100 | 100 |
| 170 | 100 | 100 | 100 | 100 | 100 | 100 |
| 171 | 99.4 | 98.4 | 100 | 100 | 100 | 100 |
| 172 | 100 | 100 | 100 | 100 | 100 | 100 |
| 173 | 99.4 | 98.4 | 100 | 100 | 100 | 100 |
| 174 | 99.4 | 98.4 | 100 | 100 | 100 | 100 |
| 175 | 99.4 | 98.4 | 100 | 100 | 100 | 100 |
| 176 | 100 | 100 | 100 | 100 | 100 | 100 |
| 177 | 100 | 100 | 100 | 100 | 100 | 100 |
| 178 | 100 | 100 | 100 | 100 | 100 | 100 |
| 179 | 99.4 | 98.4 | 100 | 100 | 100 | 100 |
| 180 | 100 | 100 | 100 | 100 | 100 | 100 |
| 181 | 100 | 100 | 100 | 100 | 100 | 100 |
| 182 | 99.4 | 98.4 | 100 | 100 | 100 | 100 |
| 183 | 99.4 | 98.4 | 100 | 100 | 100 | 100 |
| 184 | 99.4 | 98.4 | 100 | 100 | 100 | 100 |
| 185 | 99.4 | 98.4 | 100 | 100 | 100 | 100 |
| 186 | 100 | 100 | 100 | 100 | 100 | 100 |
| 187 | 99.4 | 98.4 | 100 | 100 | 100 | 100 |
| 188 | 99.4 | 98.4 | 100 | 100 | 100 | 100 |
| 189 | 99.4 | 98.4 | 100 | 100 | 100 | 100 |
| 190 | 100 | 100 | 100 | 100 | 100 | 100 |
| 191 | 99.4 | 98.4 | 100 | 100 | 100 | 100 |
| 192 | 99.4 | 98.4 | 100 | 100 | 100 | 100 |
| 193 | 99.4 | 98.4 | 100 | 100 | 100 | 100 |
| 194 | 100 | 100 | 100 | 100 | 100 | 100 |
| 195 | 99.4 | 98.4 | 100 | 100 | 100 | 100 |
| 196 | 100 | 100 | 100 | 100 | 100 | 100 |
| 197 | 99.4 | 98.4 | 100 | 100 | 100 | 100 |
| 198 | 99.4 | 98.4 | 100 | 100 | 100 | 100 |
| 199 | 99.4 | 98.4 | 100 | 100 | 100 | 100 |
| 1100 | 100 | 100 | 100 | 100 | 100 | 100 |
| 1101 | 99.4 | 98.4 | 100 | 100 | 100 | 100 |
| 1102 | 100 | 100 | 100 | 100 | 100 | 100 |
| 1103 | 99.4 | 98.4 | 100 | 100 | 100 | 100 |
| 1104 | 100 | 100 | 100 | 100 | 100 | 100 |
| 1105 | 99.4 | 98.4 | 100 | 100 | 100 | 100 |
| 1106 | 100 | 100 | 100 | 100 | 100 | 100 |
| 1107 | 100 | 100 | 100 | 100 | 100 | 100 |
| 1108 | 99.4 | 98.4 | 100 | 100 | 100 | 100 |
| 1109 | 100 | 100 | 100 | 100 | 100 | 100 |
| 1110 | 99.4 | 98.4 | 100 | 100 | 100 | 100 |
| 1111 | 99.4 | 98.4 | 100 | 100 | 100 | 100 |
| 1112 | 100 | 100 | 100 | 100 | 100 | 100 |
| 1113 | 100 | 100 | 100 | 100 | 100 | 100 |
| 1114 | 99.4 | 98.4 | 100 | 100 | 100 | 100 |
| 1115 | 99.4 | 98.4 | 100 | 100 | 100 | 100 |
| 1116 | 99.4 | 98.4 | 100 | 100 | 100 | 100 |
| 1117 | 99.4 | 98.4 | 100 | 100 | 100 | 100 |
| 1118 | 99.4 | 98.4 | 100 | 100 | 100 | 100 |
| 1119 | 99.4 | 98.4 | 100 | 100 | 100 | 100 |
| 1120 | 99.4 | 98.4 | 100 | 100 | 100 | 100 |
| 1121 | 100 | 100 | 100 | 100 | 100 | 100 |
| 1122 | 99.4 | 98.4 | 100 | 100 | 100 | 100 |
| 1123 | 99.4 | 98.4 | 100 | 100 | 100 | 100 |
| 1124 | 99.4 | 98.4 | 100 | 100 | 100 | 100 |
| 1125 | 100 | 100 | 100 | 100 | 100 | 100 |
| 1126 | 99.4 | 98.4 | 100 | 100 | 100 | 100 |
| 1127 | 100 | 100 | 100 | 100 | 100 | 100 |
| 1128 | 99.4 | 98.4 | 100 | 100 | 100 | 100 |
| 1129 | 99.4 | 98.4 | 100 | 100 | 100 | 100 |
| 1130 | 99.4 | 98.4 | 100 | 100 | 100 | 100 |
| 1131 | 99.4 | 98.4 | 100 | 100 | 100 | 100 |
| 1132 | 99.4 | 98.4 | 100 | 100 | 100 | 100 |
| 1133 | 99.4 | 98.4 | 100 | 100 | 100 | 100 |
| 1134 | 99.4 | 98.4 | 100 | 100 | 100 | 100 |
| 1135 | 99.4 | 98.4 | 100 | 100 | 100 | 100 |
| 1136 | 99.4 | 98.4 | 100 | 100 | 100 | 100 |
| 1137 | 99.4 | 98.4 | 100 | 100 | 100 | 100 |
| 1138 | 99.4 | 98.4 | 100 | 100 | 100 | 100 |
| 1139 | 99.4 | 98.4 | 100 | 100 | 100 | 100 |
| 1140 | 99.4 | 98.4 | 100 | 100 | 100 | 100 |
| 1141 | 99.4 | 98.4 | 100 | 100 | 100 | 100 |
| 1142 | 99.4 | 98.4 | 100 | 100 | 100 | 100 |
| 1143 | 100 | 100 | 100 | 100 | 100 | 100 |
| 1144 | 99.4 | 98.4 | 100 | 100 | 100 | 100 |
| 1145 | 99.4 | 98.4 | 100 | 100 | 100 | 100 |
| 1146 | 99.4 | 98.4 | 100 | 100 | 100 | 100 |
| 1147 | 99.4 | 98.4 | 100 | 100 | 100 | 100 |
| 1148 | 100 | 100 | 100 | 100 | 100 | 100 |
| 1149 | 99.4 | 98.4 | 100 | 100 | 100 | 100 |
| 1150 | 99.4 | 98.4 | 100 | 100 | 100 | 100 |
| 1151 | 99.4 | 98.4 | 100 | 100 | 100 | 100 |
| 1152 | 100 | 100 | 100 | 100 | 100 | 100 |
| 1153 | 99.4 | 98.4 | 100 | 100 | 100 | 100 |
| 1154 | 99.4 | 98.4 | 100 | 100 | 100 | 100 |
| 1155 | 99.4 | 98.4 | 100 | 100 | 100 | 100 |
| 1156 | 99.4 | 98.4 | 100 | 100 | 100 | 100 |
| 1157 | 100 | 100 | 100 | 100 | 100 | 100 |
| 1158 | 99.4 | 98.4 | 100 | 100 | 100 | 100 |
| 1159 | 99.4 | 98.4 | 100 | 100 | 100 | 100 |
| 1160 | 99.4 | 98.4 | 100 | 100 | 100 | 100 |
| 1161 | 100 | 100 | 100 | 100 | 100 | 100 |
| 1162 | 100 | 100 | 100 | 100 | 100 | 100 |
| 1163 | 99.4 | 98.4 | 100 | 100 | 100 | 100 |
| 1164 | 99.4 | 98.4 | 100 | 100 | 100 | 100 |
| 1165 | 99.4 | 98.4 | 100 | 100 | 100 | 100 |
| 1166 | 99.4 | 98.4 | 100 | 100 | 100 | 100 |
| 1167 | 99.4 | 98.4 | 100 | 100 | 100 | 100 |
| 1168 | 99.4 | 98.4 | 100 | 100 | 100 | 100 |
| 1169 | 100 | 100 | 100 | 100 | 100 | 100 |
| 1170 | 99.4 | 98.4 | 100 | 100 | 100 | 100 |
| 1171 | 99.4 | 98.4 | 100 | 100 | 100 | 100 |
| 1172 | 99.4 | 98.4 | 100 | 100 | 100 | 100 |
| 1173 | 99.4 | 98.4 | 100 | 100 | 100 | 100 |
| 1174 | 99.4 | 98.4 | 100 | 100 | 100 | 100 |
| 1175 | 99.4 | 98.4 | 100 | 100 | 100 | 100 |
| 1176 | 99.4 | 98.4 | 100 | 100 | 100 | 100 |
| 1177 | 99.4 | 98.4 | 100 | 100 | 100 | 100 |
| 1178 | 99.4 | 98.4 | 100 | 100 | 100 | 100 |
| 1179 | 99.4 | 98.4 | 100 | 100 | 100 | 100 |
| 1180 | 99.4 | 98.4 | 100 | 100 | 100 | 100 |
| 1181 | 99.4 | 98.4 | 100 | 100 | 100 | 100 |
| 1182 | 99.4 | 98.4 | 100 | 100 | 100 | 100 |
| 1183 | 99.4 | 98.4 | 100 | 100 | 100 | 100 |
| 1184 | 99.4 | 98.4 | 100 | 100 | 100 | 100 |
| 1185 | 99.4 | 98.4 | 100 | 100 | 100 | 100 |
| 1186 | 100 | 100 | 100 | 100 | 100 | 100 |
| 1187 | 99.4 | 98.4 | 100 | 100 | 100 | 100 |
| 1188 | 100 | 100 | 100 | 100 | 100 | 100 |
| 1189 | 99.4 | 98.4 | 100 | 100 | 100 | 100 |
| 1190 | 99.4 | 98.4 | 100 | 100 | 100 | 100 |
| 1191 | 99.4 | 98.4 | 100 | 100 | 100 | 100 |
| 1192 | 99.4 | 98.4 | 100 | 100 | 100 | 100 |
| 1193 | 99.4 | 98.4 | 100 | 100 | 100 | 100 |
| 1194 | 99.4 | 98.4 | 100 | 100 | 100 | 100 |
| 1195 | 100 | 100 | 100 | 100 | 100 | 100 |
| 1196 | 99.4 | 98.4 | 100 | 100 | 100 | 100 |
| 1197 | 99.4 | 98.4 | 100 | 100 | 100 | 100 |
| 1198 | 99.4 | 98.4 | 100 | 100 | 100 | 100 |
| 1199 | 99.4 | 98.4 | 100 | 100 | 100 | 100 |
| 1200 | 99.4 | 98.4 | 100 | 100 | 100 | 100 |
| 1201 | 99.4 | 98.4 | 100 | 100 | 100 | 100 |
| 1202 | 99.4 | 98.4 | 100 | 100 | 100 | 100 |
| 1203 | 99.4 | 98.4 | 100 | 100 | 100 | 100 |
| 1204 | 99.4 | 98.4 | 100 | 100 | 100 | 100 |
| 1205 | 99.4 | 98.4 | 100 | 100 | 100 | 100 |
| 1206 | 99.4 | 98.4 | 100 | 100 | 100 | 100 |
| 1207 | 99.4 | 98.4 | 100 | 100 | 100 | 100 |
| 1208 | 99.4 | 98.4 | 100 | 100 | 100 | 100 |
| 1209 | 99.4 | 98.4 | 100 | 100 | 100 | 100 |
| 1210 | 99.4 | 98.4 | 100 | 100 | 100 | 100 |
| 1211 | 99.4 | 98.4 | 100 | 100 | 100 | 100 |
| 1212 | 99.4 | 98.4 | 100 | 100 | 100 | 100 |
| 1213 | 99.4 | 98.4 | 100 | 100 | 100 | 100 |
| 1214 | 100 | 100 | 100 | 100 | 100 | 100 |
| 1215 | 100 | 100 | 100 | 100 | 100 | 100 |
| 1216 | 99.4 | 98.4 | 100 | 100 | 100 | 100 |
| 1217 | 99.4 | 98.4 | 100 | 100 | 100 | 100 |
| 1218 | 99.4 | 98.4 | 100 | 100 | 100 | 100 |
| 1219 | 100 | 100 | 100 | 100 | 100 | 100 |
| 1220 | 100 | 100 | 100 | 100 | 100 | 100 |
| 1221 | 99.4 | 98.4 | 100 | 100 | 100 | 100 |
| 1222 | 99.4 | 98.4 | 100 | 100 | 100 | 100 |
| 1223 | 99.4 | 98.4 | 100 | 100 | 100 | 100 |
| 1224 | 99.4 | 98.4 | 100 | 100 | 100 | 100 |
| 1225 | 100 | 100 | 100 | 100 | 100 | 100 |
| 1226 | 99.4 | 98.4 | 100 | 100 | 100 | 100 |
| 1227 | 99.4 | 98.4 | 100 | 100 | 100 | 100 |
| 1228 | 99.4 | 98.4 | 100 | 100 | 100 | 100 |
| 1229 | 99.4 | 98.4 | 100 | 100 | 100 | 100 |
| 1230 | 99.4 | 98.4 | 100 | 100 | 100 | 100 |
| 1231 | 99.4 | 98.4 | 100 | 100 | 100 | 100 |
| 1232 | 99.4 | 98.4 | 100 | 100 | 100 | 100 |
| 1233 | 99.4 | 98.4 | 100 | 100 | 100 | 100 |
| 1234 | 99.4 | 98.4 | 100 | 100 | 100 | 100 |
| 1235 | 99.4 | 98.4 | 100 | 100 | 100 | 100 |
| 1236 | 100 | 100 | 100 | 100 | 100 | 100 |
| 1237 | 100 | 100 | 100 | 100 | 100 | 100 |
| 1238 | 100 | 100 | 100 | 100 | 100 | 100 |
| 1239 | 100 | 100 | 100 | 100 | 100 | 100 |
| 1240 | 100 | 100 | 100 | 100 | 100 | 100 |
| 1241 | 100 | 100 | 100 | 100 | 100 | 100 |
| 1242 | 100 | 100 | 100 | 100 | 100 | 100 |
| 1243 | 100 | 100 | 100 | 100 | 100 | 100 |
| 1244 | 100 | 100 | 100 | 100 | 100 | 100 |
| 1245 | 99.4 | 98.4 | 100 | 100 | 100 | 100 |
| 1246 | 99.4 | 98.4 | 100 | 100 | 100 | 100 |
| 1247 | 99.4 | 98.4 | 100 | 100 | 100 | 100 |
| 1248 | 99.4 | 98.4 | 100 | 100 | 100 | 100 |
| 1249 | 99.4 | 98.4 | 100 | 100 | 100 | 100 |
| 1250 | 99.4 | 98.4 | 100 | 100 | 100 | 100 |
| 1251 | 99.4 | 98.4 | 100 | 100 | 100 | 100 |

TABLE 7

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 3_20 | 100 | 100 | 100 | 100 | 100 | 100 |
| 6_20 | 100 | 100 | 100 | 100 | 100 | 100 |
| 7_20 | 100 | 100 | 100 | 100 | 100 | 100 |
| 10_20 | 100 | 100 | 100 | 100 | 100 | 100 |
| 20_22 | 98.8 | 96.7 | 100 | 98.8 | 96.8 | 100 |
| 20_238 | 98.8 | 96.7 | 100 | 98.8 | 96.8 | 100 |
| 20_239 | 100 | 100 | 100 | 100 | 100 | 100 |
| 12_24 | 100 | 100 | 100 | 98.8 | 96.8 | 100 |
| 20_24 | 100 | 100 | 100 | 98.8 | 96.8 | 100 |
| 24_27 | 98.8 | 96.8 | 100 | 98.8 | 96.8 | 100 |
| 24_33 | 100 | 100 | 100 | 100 | 100 | 100 |
| 24_236 | 100 | 100 | 100 | 98.8 | 96.8 | 100 |
| 24_240 | 100 | 100 | 100 | 100 | 100 | 100 |
| 3_26 | 100 | 100 | 100 | 100 | 100 | 100 |
| 12_26 | 100 | 100 | 100 | 98.8 | 96.8 | 100 |
| 13_26 | 100 | 100 | 100 | 98.8 | 96.8 | 100 |
| 17_26 | 100 | 100 | 100 | 98.8 | 100 | 98 |
| 19_26 | 100 | 100 | 100 | 98.8 | 100 | 98 |
| 3_27 | 100 | 100 | 100 | 100 | 100 | 100 |
| 5_27 | 100 | 100 | 100 | 100 | 100 | 100 |
| 13_27 | 99.4 | 98.4 | 100 | 100 | 100 | 100 |
| 20_27 | 98.1 | 96.7 | 99 | 100 | 100 | 100 |
| 26_27 | 99.4 | 98.4 | 100 | 96.3 | 93.5 | 98 |
| 27_120 | 98.8 | 98.4 | 99 | 100 | 100 | 100 |
| 27_206 | 98.1 | 95.2 | 100 | 97.5 | 96.8 | 98 |
| 27_237 | 99.4 | 98.4 | 100 | 100 | 100 | 100 |
| 3_30 | 100 | 100 | 100 | 100 | 100 | 100 |
| 17_30 | 99.4 | 98.4 | 100 | 100 | 100 | 100 |
| 27_30 | 100 | 100 | 100 | 98.8 | 96.8 | 100 |
| 27_33 | 98.8 | 98.4 | 99 | 100 | 100 | 100 |
| 30_39 | 99.4 | 98.4 | 100 | 100 | 100 | 100 |
| 30_117 | 99.4 | 98.4 | 100 | 98.8 | 96.8 | 100 |
| 3_33 | 99.4 | 98.4 | 100 | 100 | 100 | 100 |
| 7_33 | 100 | 100 | 100 | 100 | 100 | 100 |
| 10_33 | 100 | 100 | 100 | 100 | 100 | 100 |
| 11_33 | 100 | 100 | 100 | 98.8 | 100 | 98 |
| 13_33 | 100 | 100 | 100 | 100 | 100 | 100 |
| 25_33 | 100 | 100 | 100 | 98.8 | 100 | 98 |
| 33_244 | 100 | 100 | 100 | 100 | 100 | 100 |
| 3_182 | 99.4 | 98.4 | 100 | 100 | 100 | 100 |
| 6_182 | 99.4 | 98.4 | 100 | 100 | 100 | 100 |
| 7_182 | 100 | 100 | 100 | 100 | 100 | 100 |
| 12_182 | 100 | 100 | 100 | 100 | 100 | 100 |
| 27_182 | 98.1 | 96.8 | 99 | 97.5 | 96.8 | 98 |
| 182_236 | 100 | 100 | 100 | 98.8 | 96.8 | 100 |
| 2_194 | 99.4 | 98.4 | 100 | 100 | 100 | 100 |
| 7_194 | 100 | 100 | 100 | 100 | 100 | 100 |
| 27_194 | 98.1 | 95.2 | 100 | 97.5 | 96.8 | 98 |
| 194_236 | 100 | 100 | 100 | 98.8 | 96.8 | 100 |
| 2_206 | 99.4 | 98.4 | 100 | 100 | 100 | 100 |
| 7_206 | 100 | 100 | 100 | 100 | 100 | 100 |
| 206_236 | 100 | 100 | 100 | 98.8 | 96.8 | 100 |
| 2_208 | 99.4 | 98.4 | 100 | 100 | 100 | 100 |
| 7_208 | 100 | 100 | 100 | 100 | 100 | 100 |
| 13_208 | 99.4 | 98.4 | 100 | 100 | 100 | 100 |
| 20_208 | 98.1 | 96.7 | 99 | 97.5 | 93.5 | 100 |
| 27_208 | 98.1 | 95.2 | 100 | 98.8 | 96.8 | 100 |

### Example 3

<Selection of Gene Markers Using all Samples and Method for Evaluating Breast Cancer Discriminant Performance of Acquired Gene Markers>

In this Example, the samples of the training cohort and the validation cohort used in Examples 1 and 2 were integrated, and selection of a gene marker and evaluation of its breast cancer discriminant performance were conducted using all of the samples.

Specifically, the miRNA expression levels in the sera of the 93 breast cancer patients and the 150 healthy subjects obtained in the preceding Reference Examples were normalized by quantile normalization. In order to acquire diagnostic markers with higher reliability, only genes having a gene expression level of 2⁶ or higher in 50% or more of the samples in either of the breast cancer patient group or the healthy subject group, were selected in the gene marker selection. In order to further acquire statistical significance for discriminating a breast cancer patient group from a healthy subject group, the P value obtained by two-tailed t-test assuming equal variance as to each gene expression level was corrected by the Bonferroni method, and genes that satisfied p<0.01 were selected as gene markers for use in explanatory variables of a discriminant. The obtained genes are described in Table 7. In this way, hsa-miR-658, hsa-miR-6842-5p, hsa-miR-6124, hsa-miR-6765-3p, hsa-miR-7106-5p, hsa-miR-4534, hsa-miR-92b-3p, hsa-miR-3135b, hsa-miR-4687-3p, hsa-miR-762, hsa-miR-3619-3p, hsa-miR-4467, hsa-miR-557, hsa-miR-1237-5p, hsa-miR-1908-5p, hsa-miR-4286, hsa-miR-6885-5ph and hsa-miR-6763-5p genes, and the nucleotide sequences of SEQ ID NOs: 252 to 269 related thereto were found in addition to the genes described in Table 2. As with the nucleotide sequences of SEQ ID NOs: 1 to 251, the results obtained about the polynucleotides represented by the nucleotide sequences of SEQ ID NOs: 252 to 269 also showed that the measurement values were significantly lower (−) or higher (+) in the breast cancer patient group than in the healthy subject group (Table 8). These results were able to be validated in the validation cohort. Thus, the presence or absence of breast cancer in the newly obtained samples can be determined by the methods described in Examples 1 and 2 by using the gene expression level measurement values described in Table 8 either alone or in combination with the gene expression level measurement values described in Table 2

TABLE 8

| | | | Expression level in |
|---|---|---|---|
| | | P value after | breast cancer |
| | | Bonferroni | patient with respect |
| SEQ ID NO: | Gene name | correction | to healthy subject |
| 1 | hsa-miR-4783-3p | 1.81 · E−151 | + |
| 2 | hsa-miR-4730 | 1.00 · E−148 | − |
| 3 | hsa-miR-1307-3p | 5.85 · E−126 | + |
| 4 | hsa-miR-4634 | 9.92 · E−114 | − |
| 5 | hsa-miR-663a | 2.55 · E−112 | − |
| 6 | hsa-miR-4532 | 8.96 · E−113 | + |
| 7 | hsa-miR-7704 | 4.82 · E−114 | + |
| 8 | hsa-miR-3178 | 5.69 · E−112 | − |
| 9 | hsa-miR-6729-5p | 3.52 · E−112 | − |
| 10 | hsa-miR-6090 | 1.64 · E−100 | − |
| 11 | hsa-miR-4732-5p | 7.04 · E−96 | − |
| 12 | hsa-miR-3184-5p | 2.08 · E−103 | + |
| 13 | hsa-miR-6727-5p | 3.11 · E−99 | + |
| 14 | hsa-miR-6088 | 4.52 · E−100 | − |
| 15 | hsa-miR-4674 | 2.03 · E−94 | + |
| 16 | hsa-miR-8073 | 5.49 · E−98 | − |
| 17 | hsa-miR-4787-5p | 1.53 · E−96 | + |
| 18 | hsa-miR-1469 | 3.95 · E−87 | + |
| 19 | hsa-miR-125a-3p | 3.12 · E−91 | − |
| 20 | hsa-miR-1233-5p | 2.43 · E−93 | + |
| 21 | hsa-miR-885-3p | 4.92 · E−87 | − |
| 22 | hsa-miR-6802-5p | 5.06 · E−87 | − |
| 23 | hsa-miR-328-5p | 6.06 · E−96 | + |
| 24 | hsa-miR-6787-5p | 2.26 · E−85 | + |
| 25 | hsa-miR-8069 | 5.26 · E−78 | + |
| 26 | hsa-miR-6875-5p | 2.38 · E−74 | + |
| 27 | hsa-miR-1246 | 1.05 · E−75 | + |
| 28 | hsa-miR-4734 | 6.82 · E−87 | − |
| 29 | hsa-miR-6757-5p | 9.57 · E−75 | + |
| 30 | hsa-miR-6756-5p | 4.28 · E−70 | − |
| 31 | hsa-miR-3665 | 5.78 · E−78 | − |
| 32 | hsa-miR-6836-3p | 7.87 · E−76 | − |
| 33 | hsa-miR-6821-5p | 1.18 · E−80 | − |
| 34 | hsa-miR-6805-5p | 3.26 · E−79 | − |
| 35 | hsa-miR-4728-5p | 9.76 · E−65 | + |
| 36 | hsa-miR-6726-5p | 4.07 · E−72 | + |
| 37 | hsa-miR-197-5p | 5.63 · E−71 | − |
| 38 | hsa-miR-149-3p | 3.94 · E−70 | + |
| 39 | hsa-miR-6850-5p | 2.45 · E−70 | − |
| 40 | hsa-miR-4476 | 8.32 · E−64 | + |
| 41 | hsa-miR-6858-5p | 4.09 · E−67 | − |
| 42 | hsa-miR-564 | 5.81 · E−69 | + |
| 43 | hsa-miR-4763-3p | 2.83 · E−72 | + |
| 44 | hsa-miR-575 | 1.96 · E−69 | − |
| 45 | hsa-miR-6771-5p | 1.90 · E−65 | − |
| 46 | hsa-miR-1231 | 8.50 · E−61 | − |
| 47 | hsa-miR-1908-3p | 3.70 · E−64 | + |
| 48 | hsa-miR-150-3p | 2.41 · E−58 | − |
| 49 | hsa-miR-3937 | 5.29 · E−66 | − |
| 50 | hsa-miR-887-3p | 1.78 · E−64 | − |
| 51 | hsa-miR-3940-5p | 2.92 · E−65 | − |
| 52 | hsa-miR-4741 | 1.21 · E−57 | + |
| 53 | hsa-miR-6808-5p | 5.95 · E−62 | − |
| 54 | hsa-miR-6869-5p | 9.36 · E−66 | − |
| 55 | hsa-miR-5090 | 2.20 · E−62 | − |
| 56 | hsa-miR-615-5p | 1.59 · E−59 | − |
| 57 | hsa-miR-8072 | 1.93 · E−53 | + |
| 58 | hsa-miR-128-l-5p | 2.45 · E−57 | + |
| 59 | hsa-miR-1238-5p | 3.00 · E−60 | − |
| 60 | hsa-miR-365a-5p | 5.03 · E−59 | + |
| 61 | hsa-miR-204-3p | 1.93 · E−49 | − |
| 62 | hsa-miR-4492 | 6.71 · E−60 | − |
| 63 | hsa-miR-6785-5p | 3.81 · E−56 | + |
| 64 | hsa-miR-6511a-5p | 6.21 · E−62 | + |
| 65 | hsa-miR-4525 | 2.38 · E−55 | − |
| 66 | hsa-miR-1915-5p | 8.81 · E−57 | − |
| 67 | hsa-miR-3180 | 1.19 · E−50 | − |
| 68 | hsa-miR-6879-5p | 2.38 · E−58 | − |
| 69 | hsa-miR-1199-5p | 7.83 · E−51 | − |
| 70 | hsa-miR-6746-5p | 9.76 · E−50 | − |
| 71 | hsa-miR-711 | 1.44 · E−49 | + |
| 72 | hsa-miR-663b | 4.49 · E−49 | − |
| 73 | hsa-miR-4707-3p | 2.18 · E−52 | + |
| 74 | hsa-miR-6893-5p | 2.49 · E−43 | − |
| 75 | hsa-miR-4675 | 5.39 · E−56 | + |
| 76 | hsa-miR-4638-5p | 1.64 · E−51 | + |
| 77 | hsa-miR-4651 | 6.25 · E−50 | − |
| 78 | hsa-miR-6087 | 6.15 · E−53 | − |
| 79 | hsa-miR-4665-5p | 3.35 · E−48 | − |
| 80 | hsa-miR-4758-5p | 1.52 · E−54 | − |
| 81 | hsa-miR-6887-5p | 8.54 · E−46 | − |
| 82 | hsa-miR-3620-5p | 4.32 · E−42 | − |
| 83 | hsa-miR-1909-3p | 6.17 · E−53 | + |
| 84 | hsa-miR-7641 | 2.63 · E−43 | − |
| 85 | hsa-miR-6724-5p | 3.58 · E−49 | + |
| 86 | hsa-miR-1343-3p | 1.04 · E−45 | − |
| 87 | hsa-miR-6780b-5p | 5.24 · E−50 | − |
| 88 | hsa-miR-4484 | 5.63 · E−49 | + |
| 89 | hsa-miR-4690-5p | 2.68 · E−43 | − |
| 90 | hsa-miR-4429 | 7.93 · E−44 | − |
| 91 | hsa-miR-1227-5p | 1.26 · E−46 | − |
| 92 | hsa-miR-4725-3p | 3.25 · E−44 | + |
| 93 | hsa-miR-6861-5p | 2.28 · E−46 | + |
| 94 | hsa-miR-6812-5p | 3.34 · E−37 | + |
| 95 | hsa-miR-3197 | 8.32 · E−45 | + |
| 96 | hsa-miR-8059 | 6.38 · E−41 | + |
| 97 | hsa-miR-3185 | 7.20 · E−46 | + |
| 98 | hsa-miR-4706 | 4.07 · E−39 | + |
| 99 | hsa-miR-4497 | 7.65 · E−39 | + |
| 100 | hsa-miR-3131 | 4.60 · E−42 | − |
| 101 | hsa-miR-6806-5p | 8.93 · E−34 | + |
| 102 | hsa-miR-187-5p | 5.69 · E−42 | + |
| 103 | hsa-miR-3180-3p | 1.37 · E−41 | − |
| 104 | hsa-miR-6848-5p | 9.99 · E−33 | − |
| 105 | hsa-miR-6820-5p | 2.48 · E−37 | + |
| 106 | hsa-miR-6800-5p | 2.58 · E−42 | + |
| 107 | hsa-miR-6717-5p | 2.75 · E−37 | − |
| 108 | hsa-miR-6795-5p | 2.83 · E−36 | − |
| 109 | hsa-miR-4632-5p | 1.50 · E−40 | + |
| 110 | hsa-miR-665 | 3.04 · E−37 | − |
| 111 | hsa-miR-6778-5p | 3.57 · E−36 | − |
| 112 | hsa-miR-3663-3p | 2.48 · E−39 | + |
| 113 | hsa-miR-4689 | 1.46 · E−43 | + |
| 114 | hsa-miR-211-3p | 1.36 · E−39 | + |
| 115 | hsa-miR-6511b-5p | 4.55 · E−39 | + |
| 116 | hsa-miR-4750-5p | 1.70 · E−32 | + |
| 117 | hsa-miR-6126 | 6.52 · E−41 | + |
| 118 | hsa-miR-614 | 1.36 · E−31 | + |
| 119 | hsa-miR-7110-5p | 2.57 · E−36 | − |
| 120 | hsa-miR-744-5p | 1.36 · E−32 | − |
| 121 | hsa-miR-6769a-5p | 2.68 · E−28 | + |
| 122 | hsa-miR-4792 | 3.78 · E−29 | − |
| 123 | hsa-miR-5787 | 5.62 · E−30 | + |
| 124 | hsa-miR-6798-5p | 8.69 · E−34 | − |
| 125 | hsa-miR-6781-5p | 1.34 · E−35 | + |
| 126 | hsa-miR-4419b | 3.49 · E−28 | − |
| 127 | hsa-miR-4446-3p | 2.83 · E−32 | − |
| 128 | hsa-miR-4259 | 9.25 · E−30 | − |
| 129 | hsa-miR-5572 | 7.92 · E−32 | − |
| 130 | hsa-miR-6075 | 1.06 · E−34 | − |
| 131 | hsa-miR-296-3p | 7.28 · E−27 | − |
| 132 | hsa-miR-6891-5p | 1.19 · E−28 | + |
| 133 | hsa-miR-4745-5p | 1.47 · E−30 | + |
| 134 | hsa-miR-6775-5p | 1.24 · E−28 | − |
| 135 | hsa-miR-6870-5p | 1.50 · E−29 | − |
| 136 | hsa-miR-920 | 2.07 · E−27 | + |
| 137 | hsa-miR-4530 | 4.79 · E−26 | + |
| 138 | hsa-miR-6819-5p | 4.33 · E−27 | − |
| 139 | hsa-miR-6825-5p | 3.99 · E−29 | + |
| 140 | hsa-miR-7847-3p | 1.49 · E−26 | + |
| 141 | hsa-miR-6131 | 3.83 · E−28 | − |
| 142 | hsa-miR-4433-3p | 8.75 · E−27 | − |
| 143 | hsa-miR-1228-5p | 4.12 · E−20 | − |
| 144 | hsa-miR-6743-5p | 4.05 · E−31 | − |
| 145 | hsa-miR-1268a | 1.93 · E−25 | + |
| 146 | hsa-miR-3917 | 3.71 · E−24 | − |
| 147 | hsa-miR-6786-5p | 3.71 · E−27 | + |
| 148 | hsa-miR-3154 | 1.49 · E−21 | + |
| 149 | hsa-miR-638 | 2.61 · E−28 | + |
| 150 | hsa-miR-6741-5p | 2.54 · E−24 | − |
| 151 | hsa-miR-6889-5p | 3.14 · E−26 | + |
| 152 | hsa-miR-6840-3p | 4.16 · E−20 | − |
| 153 | hsa-miR-6510-5p | 2.15 · E−19 | + |
| 154 | hsa-miR-3188 | 3.01 · E−21 | + |
| 156 | hsa-miR-5001-5p | 1.47 · E−22 | − |
| 157 | hsa-miR-1268b | 1.93 · E−20 | + |
| 158 | hsa-miR-7107-5p | 6.37 · E−21 | − |
| 159 | hsa-miR-6824-5p | 9.39 · E−21 | − |
| 160 | hsa-miR-6732-5p | 3.49 · E−17 | − |
| 161 | hsa-miR-371a-5p | 1.21 · E−19 | − |
| 162 | hsa-miR-6794-5p | 1.02 · E−15 | + |
| 163 | hsa-miR-6779-5p | 6.91 · E−17 | + |
| 164 | hsa-miR-4271 | 2.14 · E−22 | + |
| 165 | hsa-miR-5195-3p | 1.17 · E−19 | − |
| 166 | hsa-miR-6762-5p | 5.65 · E−18 | + |
| 167 | hsa-miR-939-5p | 4.70 · E−21 | + |
| 168 | hsa-miR-1247-3p | 4.05 · E−14 | + |
| 169 | hsa-miR-6777-5p | 4.37 · E−16 | − |
| 170 | hsa-miR-6722-3p | 9.50 · E−18 | − |
| 171 | hsa-miR-3656 | 1.77 · E−21 | + |
| 172 | hsa-miR-4688 | 4.75 · E−17 | − |
| 173 | hsa-miR-3195 | 3.33 · E−18 | − |
| 174 | hsa-miR-6766-5p | 1.29 · E−18 | − |
| 175 | hsa-miR-4447 | 1.50 · E−14 | + |
| 176 | hsa-miR-4656 | 7.31 · E−14 | + |
| 177 | hsa-miR-7108-5p | 1.06 · E−16 | − |
| 179 | hsa-miR-1273g-3p | 3.79 · E−17 | + |
| 180 | hsa-miR-4463 | 4.63 · E−18 | − |
| 181 | hsa-miR-2861 | 2.60 · E−15 | + |
| 182 | hsa-miR-3196 | 6.55 · E−12 | + |
| 183 | hsa-miR-6877-5p | 2.44 · E−17 | + |
| 184 | hsa-miR-3679-5p | 6.45 · E−18 | + |
| 185 | hsa-miR-4442 | 3.11 · E−15 | − |
| 186 | hsa-miR-6789-5p | 1.50 · E−14 | − |
| 187 | hsa-miR-6782-5p | 1.14 · E−13 | − |
| 188 | hsa-miR-486-3p | 8.80 · E−15 | + |
| 189 | hsa-miR-6085 | 3.88 · E−15 | + |
| 190 | hsa-miR-4746-3p | 2.87 · E−13 | − |
| 191 | hsa-miR-619-5p | 2.89 · E−12 | − |
| 192 | hsa-miR-937-5p | 2.36 · E−13 | + |
| 193 | hsa-miR-6803-5p | 2.14 · E−12 | + |
| 194 | hsa-miR-4298 | 1.25 · E−14 | + |
| 195 | hsa-miR-4454 | 9.63 · E−11 | + |
| 196 | hsa-miR-4459 | 3.66 · E−13 | − |
| 197 | hsa-miR-7150 | 1.76 · E−10 | + |
| 198 | hsa-miR-6880-5p | 3.57 · E−11 | − |
| 199 | hsa-miR-4449 | 4.62 · E−11 | + |
| 200 | hsa-miR-8063 | 4.89 · E−13 | + |
| 201 | hsa-miR-4695-5p | 5.30 · E−09 | − |
| 202 | hsa-miR-6132 | 2.15 · E−10 | + |
| 203 | hsa-miR-6829-5p | 3.96 · E−09 | − |
| 204 | hsa-miR-4486 | 2.13 · E−07 | + |
| 205 | hsa-miR-6805-3p | 8.96 · E−10 | + |
| 206 | hsa-miR-6826-5p | 7.62 · E−08 | − |
| 207 | hsa-miR-4508 | 6.79 · E−08 | + |
| 208 | hsa-miR-1343-5p | 8.96 · E−07 | + |
| 209 | hsa-miR-7114-5p | 1.55 · E−09 | + |
| 210 | hsa-miR-3622a-5p | 2.52 · E−08 | − |
| 211 | hsa-miR-6765-5p | 6.46 · E−07 | + |
| 212 | hsa-miR-7845-5p | 1.78 · E−04 | − |
| 213 | hsa-miR-3960 | 2.10 · E−10 | − |
| 214 | hsa-miR-6749-5p | 1.32 · E−07 | − |
| 215 | hsa-miR-1260b | 1.44 · E−04 | + |
| 216 | hsa-miR-6799-5p | 1.14 · E−07 | + |
| 217 | hsa-miR-4723-5p | 6.30 · E−07 | − |
| 218 | hsa-miR-6784-5p | 4.08 · E−08 | + |
| 219 | hsa-miR-5100 | 1.78 · E−06 | − |
| 220 | hsa-miR-6769b-5p | 6.44 · E−06 | + |
| 221 | hsa-miR-1207-5p | 8.21 · E−07 | − |
| 222 | hsa-miR-642a-3p | 2.06 · E−09 | − |
| 223 | hsa-miR-4505 | 3.06 · E−06 | + |
| 224 | hsa-miR-4270 | 9.73 · E−07 | + |
| 225 | hsa-miR-6721-5p | 1.84 · E−08 | − |
| 226 | hsa-miR-7111-5p | 3.24 · E−09 | + |
| 227 | hsa-miR-6791-5p | 2.88 · E−06 | − |
| 228 | hsa-miR-7109-5p | 7.96 · E−05 | + |
| 229 | hsa-miR-4258 | 1.36 · E−05 | + |
| 230 | hsa-miR-6515-3p | 3.81 · E−03 | + |
| 231 | hsa-miR-6851-5p | 1.63 · E−06 | − |
| 232 | hsa-miR-6125 | 1.36 · E−05 | − |
| 233 | hsa-miR-4749-5p | 7.65 · E−05 | − |
| 234 | hsa-miR-4726-5p | 5.88 · E−04 | + |
| 235 | hsa-miR-4513 | 4.79 · E−06 | − |
| 236 | hsa-miR-760 | 1.45 · E−111 | + |
| 237 | hsa-miR-602 | 1.06 · E−88 | − |
| 238 | hsa-miR-423-5p | 9.24 · E−79 | − |
| 239 | hsa-miR-92a-2-5p | 2.54 · E−85 | − |
| 240 | hsa-miR-16-5p | 1.34 · E−69 | + |
| 241 | hsa-miR-451a | 5.22 · E−54 | + |
| 242 | hsa-miR-135a-3p | 8.02 · E−58 | + |
| 243 | hsa-miR-486-5p | 8.22 · E−52 | + |
| 244 | hsa-miR-4257 | 4.02 · E−46 | + |
| 245 | hsa-miR-92b-5p | 1.86 · E−45 | − |
| 246 | hsa-miR-1915-3p | 4.13 · E−28 | − |
| 247 | hsa-miR-718 | 1.29 · E−25 | + |
| 248 | hsa-miR-940 | 5.23 · E−17 | − |
| 249 | hsa-miR-296-5p | 3.77 · E−16 | − |
| 250 | hsa-miR-23b-3p | 1.91 · E−04 | + |
| 251 | hsa-miR-92a-3p | 3.74 · E−07 | + |
| 252 | hsa-miR-658 | 4.76 · E−43 | + |
| 253 | hsa-miR-6842-5p | 3.63 · E−12 | − |
| 254 | hsa-miR-6124 | 8.99 · E−06 | + |
| 255 | hsa-miR-6765-3p | 2.30 · E−05 | − |
| 256 | hsa-miR-7106-5p | 4.48 · E−05 | − |
| 257 | hsa-miR-4534 | 2.10 · E−04 | − |
| 258 | hsa-miR-92b-3p | 2.53 · E−04 | − |
| 259 | hsa-miR-3135b | 3.35 · E−04 | + |
| 260 | hsa-miR-4687-3p | 3.90 · E−04 | + |
| 261 | hsa-miR-762 | 4.64 · E−04 | − |
| 262 | hsa-miR-3619-3p | 1.13 · E−03 | + |
| 263 | hsa-miR-4467 | 1.30 · E−03 | − |
| 264 | hsa-miR-557 | 1.49 · E−03 | + |
| 265 | hsa-miR-1237-5p | 1.57 · E−03 | + |
| 266 | hsa-miR-1908-5p | 4.45 · E−03 | − |
| 267 | hsa-miR-4286 | 5.37 · E−03 | − |
| 268 | hsa-miR-6885-5p | 7.57 · E−03 | + |
| 269 | hsa-miR-6763-5p | 9.02 · E−03 | + |

### Example 4

<Method for Evaluating Breast Cancer-Specific Discriminant Performance by Combination of Multiple Gene Markers Using Samples in the Validation Cohort>

In this Example, gene expression levels of miRNAs in sera were compared between breast cancer patients and a control group consisting of healthy subjects and prostate cancer patients in the same way as the method described in Example 1 using the gene markers selected in Example 1 with respect to the training cohort described in Reference Example 2, to select a gene marker for diagnosis. The polynucleotides consisting of the nucleotide sequences represented by SEQ ID NOs: 851 to 856 thus newly selected were further combined with the polynucleotides consisting of the nucleotide sequences represented by SEQ ID NOs: 1 to 269 to study a method for evaluating breast cancer-specific discriminant performance.

Specifically, first, the miRNA expression levels in the training cohort and the validation cohort obtained in Reference Example 2 mentioned above were combined and normalized by quantile normalization. Next, Fisher's discriminant analysis was conducted as to combinations of 1 to 2 measurement values that comprise at least one of the measurement values of the polynucleotides consisting of the nucleotide sequences represented by SEQ ID NOs: 1 to 269, and 851 to 856 to construct a discriminant for determining the presence or absence of breast cancer. Next, accuracy, sensitivity, and specificity in the validation cohort were calculated using the discriminant thus prepared, with a positive sample group that is the breast cancer patient group and a negative sample group that is a combination of the healthy subject group and the prostate cancer patient group. The discriminant performance of the selected polynucleotides was validated using independent samples.

Most of polynucleotides consisting of the nucleotide sequences represented by these SEQ ID NOs (SEQ ID NOs: 1 to 269, and 851 to 856 corresponding to the miRNA markers of Table 1) or complementary sequences thereof mentioned above were able to provide relatively high accuracy, sensitivity, and specificity in the determination of the presence or absence of breast cancer, and furthermore, were able to specifically discriminate breast cancer from the other cancers.

At least one polynucleotide selected from the group consisting of polynucleotides consisting of the nucleotide sequences represented by SEQ ID NOs: 1, 2, 3, 4, 5, 6, 7, 8, 9, 11, 12, 13, 14, 15, 16, 17, 20, 21, 22, 23, 24, 25, 26, 27, 29, 30, 31, 32, 33, 34, 35, 36, 37, 38, 39, 41, 43, 45, 46, 47, 49, 50, 51, 52, 53, 54, 55, 58, 59, 60, 62, 63, 64, 65, 67, 68, 69, 70, 71, 72, 73, 75, 77, 79, 80, 81, 82, 83, 86, 88, 89, 90, 92, 93, 94, 96, 98, 99, 100, 103, 104, 106, 107, 108, 110, 111, 113, 114, 115, 116, 118, 119, 121, 122, 124, 125, 126, 127, 128, 129, 130, 131, 132, 133, 135, 136, 139, 140, 143, 145, 146, 147, 149, 150, 155, 157, 160, 161, 165, 167, 171, 173, 174, 175, 177, 178, 181, 182, 186, 190, 193, 194, 199, 204, 205, 206, 208, 211, 218, 225, 232, 236, 237, 238, 239, 242, 243, 244, 246, 247, 252, 260, 265, 266, 851, 852, 853, 854, 855 and 856, or complementary sequences thereof (the cancer type-specific polynucleotide group 1) was capable of specifically binding to the target marker. Among the combinations of multiple polynucleotides selected from the cancer type-specific polynucleotide group 1, particularly, combinations comprising at least one polynucleotide selected from the group consisting of polynucleotides consisting of the nucleotide sequences represented by SEQ ID NOs: 1, 2, 3, 4, 5, 6, 7, 8, 9, 11, 12, 13, 14, 15, 16, 17, 20, 21, 22, 23, 24, 25, 26, 27, 29, 30, 31, 32, 33, 34, 35, 36, 37, 38, 39, 41, 43, 45, 46, 47, 49, 50, 51, 52, 54, 55, 58, 59, 60, 62, 63, 64, 65, 67, 68, 69, 71, 72, 73, 75, 77, 79, 80, 82, 83, 86, 88, 92, 93, 96, 99, 103, 104, 106, 110, 111, 114, 116, 118, 119, 122, 124, 125, 127, 130, 132, 133, 135, 139, 143, 145, 147, 149, 157, 160, 173, 177, 181, 182, 186, 211, 218, 232, 236, 237, 238, 239, 242, 243, 246, 247, 260, 266, 851, 852, 853 and 854, or complementary sequences thereof (the cancer type-specific polynucleotide group 2) were able to specifically discriminate breast cancer from the other cancer with high accuracy.

The number of the polynucleotides with cancer type specificity in the combination mentioned above can be 1, 2, 3, 4, 5, 6, 7, 8, 9, 10 or more for the combination. The combinations of 2 or more of these polynucleotides were able to exhibit discriminant accuracy of 95% or higher.

Specifically, the discriminant accuracy of the measurement using combinations of one or two polynucleotide(s) that consists of a nucleotide sequence represented by any of SEQ ID NOs: 1 to 269 and 851 to 856 or a complementary sequence thereof is shown in Table 9.

For example, the combinations of SEQ ID NOs: 2 and 1, SEQ ID NOs: 2 and 237, SEQ ID NOs: 2 and 4, SEQ ID NOs: 2 and 3, SEQ ID NOs: 2 and 51, SEQ ID NOs: 1 and 237, SEQ ID NOs: 1 and 4, SEQ ID NOs: 1 and 3, SEQ ID NOs: 1 and 51, SEQ ID NOs: 1 and 6, SEQ ID NOs: 4 and 237, SEQ ID NOs: 3 and 237, SEQ ID NOs: 51 and 237, SEQ ID NOs: 237 and 6, SEQ ID NOs: 237 and 12, SEQ ID NOs: 3 and 4, SEQ ID NOs: 4 and 51, SEQ ID NOs: 4 and 6, SEQ ID NOs: 4 and 12, SEQ ID NOs: 4 and 15, SEQ ID NOs: 3 and 51, SEQ ID NOs: 3 and 6, SEQ ID NOs: 3 and 12, SEQ ID NOs: 3 and 15, SEQ ID NOs: 3 and 8, SEQ ID NOs: 51 and 6, SEQ ID NOs: 51 and 12, SEQ ID NOs: 51 and 15, SEQ ID NOs: 51 and 8, SEQ ID NOs: 51 and 34, SEQ ID NOs: 2 and 6, SEQ ID NOs: 12 and 6, SEQ ID NOs: 15 and 6, SEQ ID NOs: 8 and 6, SEQ ID NOs: 6 and 34, SEQ ID NOs: 2 and 12, SEQ ID NOs: 1 and 12, SEQ ID NOs: 12 and 15, SEQ ID NOs: 8 and 12, SEQ ID NOs: 12 and 34, SEQ ID NOs: 2 and 15, SEQ ID NOs: 1 and 15, SEQ ID NOs: 237 and 15, SEQ ID NOs: 8 and 15, SEQ ID NOs: 15 and 34, SEQ ID NOs: 2 and 8, SEQ ID NOs: 1 and 8, SEQ ID NOs: 237 and 8, SEQ ID NOs: 4 and 8, SEQ ID NOs: 8 and 34, SEQ ID NOs: 2 and 34, SEQ ID NOs: 1 and 34, SEQ ID NOs: 237 and 34, SEQ ID NOs: 4 and 34, SEQ ID NOs: 3 and 34, SEQ ID NOs: 2 and 9, SEQ ID NOs: 1 and 9, SEQ ID NOs: 9 and 237, SEQ ID NOs: 4 and 9, SEQ ID NOs: 3 and 9, SEQ ID NOs: 2 and 143, SEQ ID NOs: 1 and 143, SEQ ID NOs: 237 and 143, SEQ ID NOs: 4 and 143, SEQ ID NOs: 3 and 143, SEQ ID NOs: 2 and 13, SEQ ID NOs: 1 and 13, SEQ ID NOs: 237 and 13, SEQ ID NOs: 4 and 13, SEQ ID NOs: 3 and 13, SEQ ID NOs: 2 and 125, SEQ ID NOs: 1 and 125, SEQ ID NOs: 237 and 125, SEQ ID NOs: 4 and 125, SEQ ID NOs: 3 and 125, SEQ ID NOs: 2 and 236, SEQ ID NOs: 1 and 236, SEQ ID NOs: 237 and 236, SEQ ID NOs: 4 and 236, SEQ ID NOs: 3 and 236, SEQ ID NOs: 2 and 46, SEQ ID NOs: 1 and 46, SEQ ID NOs: 237 and 46, SEQ ID NOs: 4 and 46, SEQ ID NOs: 3 and 46, SEQ ID NOs: 2 and 32, SEQ ID NOs: 1 and 32, SEQ ID NOs: 237 and 32, SEQ ID NOs: 4 and 32, SEQ ID NOs: 3 and 32, SEQ ID NOs: 2 and 62, SEQ ID NOs: 1 and 62, SEQ ID NOs: 237 and 62, SEQ ID NOs: 4 and 62, SEQ ID NOs: 3 and 62, SEQ ID NOs: 2 and 88, SEQ ID NOs: 1 and 88, SEQ ID NOs: 237 and 88, SEQ ID NOs: 4 and 88, SEQ ID NOs: 3 and 88, SEQ ID NOs: 2 and 52, SEQ ID NOs: 1 and 52, SEQ ID NOs: 237 and 52, SEQ ID NOs: 4 and 52, SEQ ID NOs: 3 and 52, SEQ ID NOs: 2 and 7, SEQ ID NOs: 1 and 7, SEQ ID NOs: 237 and 7, SEQ ID NOs: 4 and 7, SEQ ID NOs: 3 and 7, SEQ ID NOs: 2 and 26, SEQ ID NOs: 1 and 26, SEQ ID NOs: 237 and 26, SEQ ID NOs: 4 and 26, SEQ ID NOs: 3 and 26, SEQ ID NOs: 2 and 25, SEQ ID NOs: 1 and 25, SEQ ID NOs: 237 and 25, SEQ ID NOs: 4 and 25, SEQ ID NOs: 3 and 25, SEQ ID NOs: 2 and 54, SEQ ID NOs: 1 and 54, SEQ ID NOs: 237 and 54, SEQ ID NOs: 4 and 54, SEQ ID NOs: 3 and 54, SEQ ID NOs: 2 and 92, SEQ ID NOs: 1 and 92, SEQ ID NOs: 237 and 92, SEQ ID NOs: 4 and 92, SEQ ID NOs: 3 and 92, SEQ ID NOs: 2 and 14, SEQ ID NOs: 1 and 14, SEQ ID NOs: 237 and 14, SEQ ID NOs: 4 and 14, SEQ ID NOs: 3 and 14, SEQ ID NOs: 2 and 242, SEQ ID NOs: 1 and 242, SEQ ID NOs: 237 and 242, SEQ ID NOs: 4 and 242, SEQ ID NOs: 3 and 242, SEQ ID NOs: 2 and 47, SEQ ID NOs: 1 and 47, SEQ ID NOs: 237 and 47, SEQ ID NOs: 4 and 47, SEQ ID NOs: 3 and 47, SEQ ID NOs: 2 and 45, SEQ ID NOs: 1 and 45, SEQ ID NOs: 237 and 45, SEQ ID NOs: 4 and 45, SEQ ID NOs: 3 and 45, SEQ ID NOs: 2 and 39, SEQ ID NOs: 1 and 39, SEQ ID NOs: 237 and 39, SEQ ID NOs: 4 and 39, SEQ ID NOs: 3 and 39, SEQ ID NOs: 2 and 21, SEQ ID NOs: 1 and 21, SEQ ID NOs: 237 and 21, SEQ ID NOs: 4 and 21, SEQ ID NOs: 3 and 21, SEQ ID NOs: 2 and 17, SEQ ID NOs: 1 and 17, SEQ ID NOs: 237 and 17, SEQ ID NOs: 4 and 17, SEQ ID NOs: 3 and 17, SEQ ID NOs: 2 and 83, SEQ ID NOs: 1 and 83, SEQ ID NOs: 237 and 83, SEQ ID NOs: 4 and 83, SEQ ID NOs: 3 and 83, SEQ ID NOs: 2 and 149, SEQ ID NOs: 1 and 149, SEQ ID NOs: 237 and 149, SEQ ID NOs: 4 and 149, SEQ ID NOs: 3 and 149, SEQ ID NOs: 2 and 246, SEQ ID NOs: 1 and 246, SEQ ID NOs: 237 and 246, SEQ ID NOs: 4 and 246, SEQ ID NOs: 3 and 246, SEQ ID NOs: 2 and 22, SEQ ID NOs: 1 and 22, SEQ ID NOs: 237 and 22, SEQ ID NOs: 4 and 22, SEQ ID NOs: 3 and 22, SEQ ID NOs: 2 and 55, SEQ ID NOs: 1 and 55, SEQ ID NOs: 237 and 55, SEQ ID NOs: 4 and 55, SEQ ID NOs: 3 and 55, SEQ ID NOs: 2 and 182, SEQ ID NOs: 1 and 182, SEQ ID NOs: 237 and 182, SEQ ID NOs: 4 and 182, SEQ ID NOs: 3 and 182, SEQ ID NOs: 2 and 73, SEQ ID NOs: 1 and 73, SEQ ID NOs: 237 and 73, SEQ ID NOs: 4 and 73, SEQ ID NOs: 3 and 73, SEQ ID NOs: 2 and 77, SEQ ID NOs: 1 and 77, SEQ ID NOs: 237 and 77, SEQ ID NOs: 4 and 77, SEQ ID NOs: 3 and 77, SEQ ID NOs: 2 and 24, SEQ ID NOs: 1 and 24, SEQ ID NOs: 237 and 24, SEQ ID NOs: 4 and 24, SEQ ID NOs: 3 and 24, SEQ ID NOs: 2 and 103, SEQ ID NOs: 1 and 103, SEQ ID NOs: 237 and 103, SEQ ID NOs: 4 and 103, SEQ ID NOs: 3 and 103, SEQ ID NOs: 2 and 49, SEQ ID NOs: 1 and 49, SEQ ID NOs: 237 and 49, SEQ ID NOs: 4 and 49, SEQ ID NOs: 3 and 49, SEQ ID NOs: 2 and 239, SEQ ID NOs: 1 and 239, SEQ ID NOs: 237 and 239, SEQ ID NOs: 4 and 239, SEQ ID NOs: 3 and 239, SEQ ID NOs: 2 and 23, SEQ ID NOs: 1 and 23, SEQ ID NOs: 237 and 23, SEQ ID NOs: 4 and 23, SEQ ID NOs: 3 and 23, SEQ ID NOs: 2 and 58, SEQ ID NOs: 1 and 58, SEQ ID NOs: 237 and 58, SEQ ID NOs: 4 and 58, SEQ ID NOs: 3 and 58, SEQ ID NOs: 2 and 211, SEQ ID NOs: 1 and 211, SEQ ID NOs: 237 and 211, SEQ ID NOs: 4 and 211, SEQ ID NOs: 3 and 211, SEQ ID NOs: 2 and 147, SEQ ID NOs: 1 and 147, SEQ ID NOs: 237 and 147, SEQ ID NOs: 4 and 147, SEQ ID NOs: 3 and 147, SEQ ID NOs: 2 and 65, SEQ ID NOs: 1 and 65, SEQ ID NOs: 237 and 65, SEQ ID NOs: 4 and 65, SEQ ID NOs: 3 and 65, SEQ ID NOs: 2 and 31, SEQ ID NOs: 1 and 31, SEQ ID NOs: 237 and 31, SEQ ID NOs: 4 and 31, SEQ ID NOs: 3 and 31, SEQ ID NOs: 2 and 72, SEQ ID NOs: 1 and 72, SEQ ID NOs: 237 and 72, SEQ ID NOs: 4 and 72, SEQ ID NOs: 3 and 72, SEQ ID NOs: 2 and 63, SEQ ID NOs: 1 and 63, SEQ ID NOs: 237 and 63, SEQ ID NOs: 4 and 63, SEQ ID NOs: 3 and 63, SEQ ID NOs: 2 and 80, SEQ ID NOs: 1 and 80, SEQ ID NOs: 237 and 80, SEQ ID NOs: 4 and 80, SEQ ID NOs: 3 and 80, SEQ ID NOs: 2 and 37, SEQ ID NOs: 1 and 37, SEQ ID NOs: 237 and 37, SEQ ID NOs: 4 and 37, SEQ ID NOs: 3 and 37, SEQ ID NOs: 2 and 67, SEQ ID NOs: 1 and 67, SEQ ID NOs: 237 and 67, SEQ ID NOs: 4 and 67, SEQ ID NOs: 3 and 67, SEQ ID NOs: 2 and 232, SEQ ID NOs: 1 and 232, SEQ ID NOs: 237 and 232, SEQ ID NOs: 4 and 232, SEQ ID NOs: 3 and 232, SEQ ID NOs: 2 and 127, SEQ ID NOs: 1 and 127, SEQ ID NOs: 237 and 127, SEQ ID NOs: 4 and 127, SEQ ID NOs: 3 and 127, SEQ ID NOs: 2 and 145, SEQ ID NOs: 1 and 145, SEQ ID NOs: 237 and 145, SEQ ID NOs: 4 and 145, SEQ ID NOs: 3 and 145, SEQ ID NOs: 2 and 16, SEQ ID NOs: 1 and 16, SEQ ID NOs: 237 and 16, SEQ ID NOs: 4 and 16, SEQ ID NOs: 3 and 16, SEQ ID NOs: 2 and 11, SEQ ID NOs: 1 and 11, SEQ ID NOs: 237 and 11, SEQ ID NOs: 4 and 11, SEQ ID NOs: 3 and 11, SEQ ID NOs: 2 and 186, SEQ ID NOs: 1 and 186, SEQ ID NOs: 237 and 186, SEQ ID NOs: 4 and 186, SEQ ID NOs: 3 and 186, SEQ ID NOs: 2 and 50, SEQ ID NOs: 1 and 50, SEQ ID NOs: 237 and 50, SEQ ID NOs: 4 and 50, SEQ ID NOs: 3 and 50, SEQ ID NOs: 2 and 69, SEQ ID NOs: 1 and 69, SEQ ID NOs: 237 and 69, SEQ ID NOs: 4 and 69, SEQ ID NOs: 3 and 69, SEQ ID NOs: 2 and 33, SEQ ID NOs: 1 and 33, SEQ ID NOs: 237 and 33, SEQ ID NOs: 4 and 33, SEQ ID NOs: 3 and 33, SEQ ID NOs: 2 and 247, SEQ ID NOs: 1 and 247, SEQ ID NOs: 237 and 247, SEQ ID NOs: 4 and 247, SEQ ID NOs: 3 and 247, SEQ ID NOs: 2 and 36, SEQ ID NOs: 1 and 36, SEQ ID NOs: 237 and 36, SEQ ID NOs: 4 and 36, SEQ ID NOs: 3 and 36, SEQ ID NOs: 2 and 218, SEQ ID NOs: 1 and 218, SEQ ID NOs: 237 and 218, SEQ ID NOs: 4 and 218, SEQ ID NOs: 3 and 218, SEQ ID NOs: 2 and 43, SEQ ID NOs: 1 and 43, SEQ ID NOs: 237 and 43, SEQ ID NOs: 4 and 43, SEQ ID NOs: 3 and 43, SEQ ID NOs: 2 and 29, SEQ ID NOs: 1 and 29, SEQ ID NOs: 237 and 29, SEQ ID NOs: 4 and 29, SEQ ID NOs: 3 and 29, SEQ ID NOs: 2 and 110, SEQ ID NOs: 1 and 110, SEQ ID NOs: 237 and 110, SEQ ID NOs: 4 and 110, SEQ ID NOs: 3 and 110, SEQ ID NOs: 2 and 20, SEQ ID NOs: 1 and 20, SEQ ID NOs: 237 and 20, SEQ ID NOs: 4 and 20, SEQ ID NOs: 3 and 20, SEQ ID NOs: 2 and 157, SEQ ID NOs: 1 and 157, SEQ ID NOs: 237 and 157, SEQ ID NOs: 4 and 157, SEQ ID NOs: 3 and 157, SEQ ID NOs: 2 and 75, SEQ ID NOs: 1 and 75, SEQ ID NOs: 237 and 75, SEQ ID NOs: 4 and 75, SEQ ID NOs: 3 and 75, SEQ ID NOs: 2 and 82, SEQ ID NOs: 1 and 82, SEQ ID NOs: 237 and 82, SEQ ID NOs: 4 and 82, SEQ ID NOs: 3 and 82, SEQ ID NOs: 2 and 106, SEQ ID NOs: 1 and 106, SEQ ID NOs: 237 and 106, SEQ ID NOs: 4 and 106, SEQ ID NOs: 3 and 106, SEQ ID NOs: 2 and 111, SEQ ID NOs: 1 and 111, SEQ ID NOs: 237 and 111, SEQ ID NOs: 4 and 111, SEQ ID NOs: 3 and 111, SEQ ID NOs: 2 and 96, SEQ ID NOs: 1 and 96, SEQ ID NOs: 237 and 96, SEQ ID NOs: 4 and 96, SEQ ID NOs: 3 and 96, SEQ ID NOs: 2 and 266, SEQ ID NOs: 1 and 266, SEQ ID NOs: 237 and 266, SEQ ID NOs: 4 and 266, SEQ ID NOs: 3 and 266, SEQ ID NOs: 2 and 124, SEQ ID NOs: 1 and 124, SEQ ID NOs: 237 and 124, SEQ ID NOs: 4 and 124, SEQ ID NOs: 3 and 124, SEQ ID NOs: 2 and 68, SEQ ID NOs: 1 and 68, SEQ ID NOs: 237 and 68, SEQ ID NOs: 4 and 68, SEQ ID NOs: 3 and 68, SEQ ID NOs: 2 and 71, SEQ ID NOs: 1 and 71, SEQ ID NOs: 237 and 71, SEQ ID NOs: 4 and 71, SEQ ID NOs: 3 and 71, SEQ ID NOs: 2 and 35, SEQ ID NOs: 1 and 35, SEQ ID NOs: 237 and 35, SEQ ID NOs: 4 and 35, SEQ ID NOs: 3 and 35, SEQ ID NOs: 2 and 173, SEQ ID NOs: 1 and 173, SEQ ID NOs: 237 and 173, SEQ ID NOs: 4 and 173, SEQ ID NOs: 3 and 173, SEQ ID NOs: 2 and 5, SEQ ID NOs: 1 and 5, SEQ ID NOs: 237 and 5, SEQ ID NOs: 4 and 5, SEQ ID NOs: 3 and 5, SEQ ID NOs: 2 and 851, SEQ ID NOs: 1 and 851, SEQ ID NOs: 237 and 851, SEQ ID NOs: 4 and 851, SEQ ID NOs: 3 and 851, SEQ ID NOs: 2 and 852, SEQ ID NOs: 1 and 852, SEQ ID NOs: 237 and 852, SEQ ID NOs: 4 and 852, SEQ ID NOs: 3 and 852, SEQ ID NOs: 2 and 30, SEQ ID NOs: 1 and 30, SEQ ID NOs: 237 and 30, SEQ ID NOs: 4 and 30, SEQ ID NOs: 3 and 30, SEQ ID NOs: 2 and 93, SEQ ID NOs: 1 and 93, SEQ ID NOs: 237 and 93, SEQ ID NOs: 4 and 93, SEQ ID NOs: 3 and 93, SEQ ID NOs: 2 and 27, SEQ ID NOs: 1 and 27, SEQ ID NOs: 237 and 27, SEQ ID NOs: 4 and 27, SEQ ID NOs: 3 and 27, SEQ ID NOs: 2 and 853, SEQ ID NOs: 1 and 853, SEQ ID NOs: 237 and 853, SEQ ID NOs: 4 and 853, SEQ ID NOs: 3 and 853, SEQ ID NOs: 2 and 238, SEQ ID NOs: 1 and 238, SEQ ID NOs: 237 and 238, SEQ ID NOs: 4 and 238, SEQ ID NOs: 3 and 238, SEQ ID NOs: 2 and 130, SEQ ID NOs: 1 and 130, SEQ ID NOs: 237 and 130, SEQ ID NOs: 4 and 130, SEQ ID NOs: 3 and 130, SEQ ID NOs: 2 and 177, SEQ ID NOs: 1 and 177, SEQ ID NOs: 237 and 177, SEQ ID NOs: 4 and 177, SEQ ID NOs: 3 and 177, SEQ ID NOs: 2 and 64, SEQ ID NOs: 1 and 64, SEQ ID NOs: 237 and 64, SEQ ID NOs: 4 and 64, SEQ ID NOs: 3 and 64, SEQ ID NOs: 2 and 114, SEQ ID NOs: 1 and 114, SEQ ID NOs: 237 and 114, SEQ ID NOs: 4 and 114, SEQ ID NOs: 3 and 114, SEQ ID NOs: 2 and 119, SEQ ID NOs: 1 and 119, SEQ ID NOs: 237 and 119, SEQ ID NOs: 4 and 119, SEQ ID NOs: 3 and 119, SEQ ID NOs: 2 and 135, SEQ ID NOs: 1 and 135, SEQ ID NOs: 237 and 135, SEQ ID NOs: 4 and 135, SEQ ID NOs: 3 and 135, SEQ ID NOs: 2 and 243, SEQ ID NOs: 1 and 243, SEQ ID NOs: 237 and 243, SEQ ID NOs: 4 and 243, SEQ ID NOs: 3 and 243, SEQ ID NOs: 2 and 122, SEQ ID NOs: 1 and 122, SEQ ID NOs: 237 and 122, SEQ ID NOs: 4 and 122, SEQ ID NOs: 3 and 122, SEQ ID NOs: 2 and 260, SEQ ID NOs: 1 and 260, SEQ ID NOs: 237 and 260, SEQ ID NOs: 4 and 260, SEQ ID NOs: 3 and 260, SEQ ID NOs: 2 and 59, SEQ ID NOs: 1 and 59, SEQ ID NOs: 237 and 59, SEQ ID NOs: 4 and 59, SEQ ID NOs: 3 and 59, SEQ ID NOs: 2 and 854, SEQ ID NOs: 1 and 854, SEQ ID NOs: 237 and 854, SEQ ID NOs: 4 and 854, SEQ ID NOs: 3 and 854, SEQ ID NOs: 2 and 132, SEQ ID NOs: 1 and 132, SEQ ID NOs: 237 and 132, SEQ ID NOs: 4 and 132, SEQ ID NOs: 3 and 132, SEQ ID NOs: 2 and 181, SEQ ID NOs: 1 and 181, SEQ ID NOs: 237 and 181, SEQ ID NOs: 4 and 181, SEQ ID NOs: 3 and 181, SEQ ID NOs: 2 and 79, SEQ ID NOs: 1 and 79, SEQ ID NOs: 237 and 79, SEQ ID NOs: 4 and 79, SEQ ID NOs: 3 and 79, SEQ ID NOs: 2 and 133, SEQ ID NOs: 1 and 133, SEQ ID NOs: 237 and 133, SEQ ID NOs: 4 and 133, SEQ ID NOs: 3 and 133, SEQ ID NOs: 2 and 41, SEQ ID NOs: 1 and 41, SEQ ID NOs: 237 and 41, SEQ ID NOs: 4 and 41, SEQ ID NOs: 3 and 41, SEQ ID NOs: 2 and 139, SEQ ID NOs: 1 and 139, SEQ ID NOs: 237 and 139, SEQ ID NOs: 4 and 139, SEQ ID NOs: 3 and 139, SEQ ID NOs: 2 and 118, SEQ ID NOs: 1 and 118, SEQ ID NOs: 237 and 118, SEQ ID NOs: 4 and 118, SEQ ID NOs: 3 and 118, SEQ ID NOs: 2 and 86, SEQ ID NOs: 1 and 86, SEQ ID NOs: 237 and 86, SEQ ID NOs: 4 and 86, SEQ ID NOs: 3 and 86, SEQ ID NOs: 2 and 60, SEQ ID NOs: 1 and 60, SEQ ID NOs: 237 and 60, SEQ ID NOs: 4 and 60, SEQ ID NOs: 3 and 60, SEQ ID NOs: 2 and 116, SEQ ID NOs: 1 and 116, SEQ ID NOs: 237 and 116, SEQ ID NOs: 4 and 116, SEQ ID NOs: 3 and 116, SEQ ID NOs: 2 and 160, SEQ ID NOs: 1 and 160, SEQ ID NOs: 237 and 160, SEQ ID NOs: 4 and 160, SEQ ID NOs: 3 and 160, SEQ ID NOs: 2 and 38, SEQ ID NOs: 1 and 38, SEQ ID NOs: 237 and 38, SEQ ID NOs: 4 and 38, SEQ ID NOs: 3 and 38, SEQ ID NOs: 2 and 99, SEQ ID NOs: 1 and 99, SEQ ID NOs: 237 and 99, SEQ ID NOs: 4 and 99, SEQ ID NOs: 3 and 99, SEQ ID NOs: 2 and 104, SEQ ID NOs: 1 and 104, SEQ ID NOs: 237 and 104, SEQ ID NOs: 4 and 104, and SEQ ID NOs: 3 and 104 were able to produce breast cancer discriminant accuracy of 95% or higher.

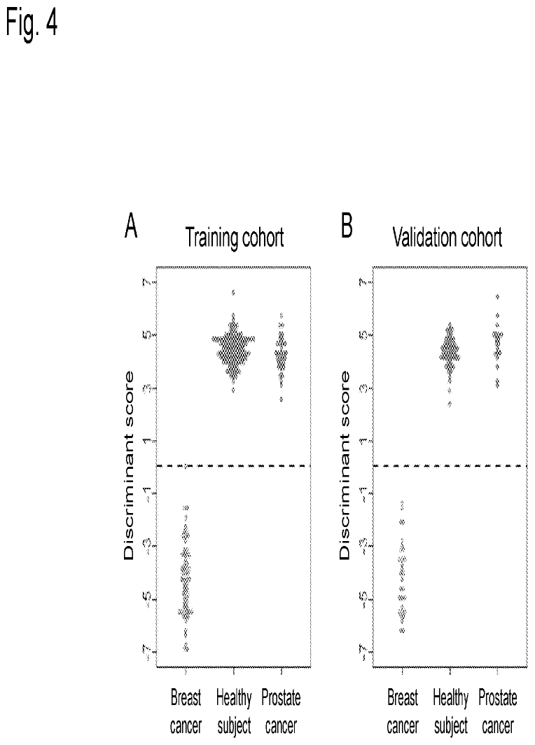

The measurement values of the nucleotide sequences represented by SEQ ID NOs: 2 and 237 were further compared among 62 breast cancer patients, 102 healthy subjects, and 33 prostate cancer patients in the training cohort. As a result, a scatter diagram that significantly separated the discriminant scores of the breast cancer patient group from those of the other groups was obtained in the training cohort (see FIG. 4A). These results were also reproducible for the validation cohort (see FIG. 4B).

TABLE 9

| | Training cohort | | | Validation cohort | | |
|---|---|---|---|---|---|---|
| | Accuracy | Sensitivity | Specificity | Accuracy | Sensitivity | Specificity |
| SEQ ID NO: | (%) | (%) | (%) | (%) | (%) | (%) |
| 2 | 99.5 | 99.5 | 99.5 | 99.5 | 99.5 | 99.5 |
| 2_1 | 100 | 100 | 100 | 100 | 100 | 100 |
| 2_237 | 99.5 | 98.4 | 100 | 100 | 100 | 100 |
| 2_4 | 99.5 | 98.4 | 100 | 100 | 100 | 100 |
| 2_3 | 100 | 100 | 100 | 100 | 100 | 100 |
| 2_51 | 99.5 | 98.4 | 100 | 100 | 100 | 100 |
| 1 | 99.5 | 99.5 | 99.5 | 99.5 | 99.5 | 99.5 |
| 1_237 | 100 | 100 | 100 | 100 | 100 | 100 |
| 1_4 | 100 | 100 | 100 | 100 | 100 | 100 |
| 1_3 | 99.5 | 98.4 | 100 | 100 | 100 | 100 |
| 1_51 | 100 | 100 | 100 | 100 | 100 | 100 |
| 1_6 | 99.5 | 98.4 | 100 | 100 | 100 | 100 |
| 237 | 98.5 | 98.5 | 98.5 | 98.5 | 98.5 | 98.5 |
| 4_237 | 99.5 | 98.4 | 100 | 99 | 96.8 | 100 |
| 3_237 | 100 | 100 | 100 | 100 | 100 | 100 |
| 51_237 | 99 | 96.8 | 100 | 100 | 100 | 100 |
| 237_6 | 100 | 100 | 100 | 100 | 100 | 100 |
| 237_12 | 100 | 100 | 100 | 99 | 96.8 | 100 |
| 4 | 99 | 99 | 99 | 99 | 99 | 99 |
| 3_4 | 100 | 100 | 100 | 100 | 100 | 100 |
| 4_51 | 99.5 | 98.4 | 100 | 100 | 100 | 100 |
| 4_6 | 100 | 100 | 100 | 100 | 100 | 100 |
| 4_12 | 100 | 100 | 100 | 99 | 96.8 | 100 |
| 4_15 | 99.5 | 98.4 | 100 | 99 | 96.8 | 100 |
| 3 | 99.5 | 99.5 | 99.5 | 99.5 | 99.5 | 99.5 |
| 3_51 | 100 | 100 | 100 | 100 | 100 | 100 |
| 3_6 | 99 | 96.8 | 100 | 100 | 100 | 100 |
| 3_12 | 100 | 100 | 100 | 100 | 100 | 100 |
| 3_15 | 100 | 100 | 100 | 100 | 100 | 100 |
| 3_8 | 100 | 100 | 100 | 100 | 100 | 100 |
| 51 | 99 | 99 | 99 | 99 | 99 | 99 |
| 51_6 | 99.5 | 100 | 99.3 | 100 | 100 | 100 |
| 51_12 | 100 | 100 | 100 | 100 | 100 | 100 |
| 51_15 | 99.5 | 98.4 | 100 | 100 | 100 | 100 |
| 51_8 | 100 | 100 | 100 | 100 | 100 | 100 |
| 51_34 | 99.5 | 98.4 | 100 | 100 | 100 | 100 |
| 6 | 98.5 | 98.5 | 98.5 | 98.5 | 98.5 | 98.5 |
| 2_6 | 100 | 100 | 100 | 100 | 100 | 100 |
| 12_6 | 100 | 100 | 100 | 100 | 100 | 100 |
| 15_6 | 100 | 100 | 100 | 100 | 100 | 100 |
| 8_6 | 100 | 100 | 100 | 100 | 100 | 100 |
| 6_34 | 100 | 100 | 100 | 100 | 100 | 100 |
| 12 | 100 | 100 | 100 | 100 | 100 | 100 |
| 2_12 | 100 | 100 | 100 | 100 | 100 | 100 |
| 1_12 | 100 | 100 | 100 | 100 | 100 | 100 |
| 12_15 | 100 | 100 | 100 | 99 | 96.8 | 100 |
| 8_12 | 100 | 100 | 100 | 100 | 100 | 100 |
| 12_34 | 100 | 100 | 100 | 100 | 100 | 100 |
| 15 | 98.5 | 98.5 | 98.5 | 98.5 | 98.5 | 98.5 |
| 2_15 | 99.5 | 98.4 | 100 | 100 | 100 | 100 |
| 1_15 | 100 | 100 | 100 | 100 | 100 | 100 |
| 237_15 | 99 | 96.8 | 100 | 99 | 96.8 | 100 |
| 8_15 | 99.5 | 98.4 | 100 | 100 | 100 | 100 |
| 15_34 | 99 | 96.8 | 100 | 100 | 100 | 100 |
| 8 | 99.5 | 99.5 | 99.5 | 99.5 | 99.5 | 99.5 |
| 2_8 | 99.5 | 98.4 | 100 | 100 | 100 | 100 |
| 1_8 | 100 | 100 | 100 | 100 | 100 | 100 |
| 237_8 | 100 | 100 | 100 | 100 | 100 | 100 |
| 4_8 | 100 | 100 | 100 | 100 | 100 | 100 |
| 8_34 | 99.5 | 98.4 | 100 | 99 | 100 | 98.5 |
| 34 | 99 | 99 | 99 | 99 | 99 | 99 |
| 2_34 | 99.5 | 98.4 | 100 | 100 | 100 | 100 |
| 1_34 | 100 | 100 | 100 | 100 | 100 | 100 |
| 237_34 | 100 | 100 | 100 | 99 | 96.8 | 100 |
| 4_34 | 100 | 100 | 100 | 100 | 100 | 100 |
| 3_34 | 100 | 100 | 100 | 100 | 100 | 100 |
| 9 | 99.5 | 99.5 | 99.5 | 99.5 | 99.5 | 99.5 |
| 2_9 | 100 | 100 | 100 | 100 | 100 | 100 |
| 1_9 | 99.5 | 98.4 | 100 | 100 | 100 | 100 |
| 9_237 | 100 | 100 | 100 | 100 | 100 | 100 |
| 4_9 | 100 | 100 | 100 | 100 | 100 | 100 |
| 3_9 | 100 | 100 | 100 | 100 | 100 | 100 |
| 143 | 98 | 98 | 98 | 98 | 98 | 98 |
| 2_143 | 100 | 100 | 100 | 100 | 100 | 100 |
| 1_143 | 100 | 100 | 100 | 100 | 100 | 100 |
| 237_143 | 100 | 100 | 100 | 99 | 96.8 | 100 |
| 4_143 | 100 | 100 | 100 | 100 | 100 | 100 |
| 3_143 | 100 | 100 | 100 | 100 | 100 | 100 |
| 13 | 99 | 99 | 99 | 99 | 99 | 99 |
| 2_13 | 99.5 | 98.4 | 100 | 100 | 100 | 100 |
| 1_13 | 99.5 | 98.4 | 100 | 100 | 100 | 100 |
| 237_13 | 100 | 100 | 100 | 100 | 100 | 100 |
| 4_13 | 99.5 | 98.4 | 100 | 100 | 100 | 100 |
| 3_13 | 100 | 100 | 100 | 100 | 100 | 100 |
| 125 | 98 | 98 | 98 | 98 | 98 | 98 |
| 2_125 | 99.5 | 98.4 | 100 | 100 | 100 | 100 |
| 1_125 | 100 | 100 | 100 | 100 | 100 | 100 |
| 237_125 | 99 | 96.8 | 100 | 99 | 96.8 | 100 |
| 4_125 | 99.5 | 98.4 | 100 | 99 | 96.8 | 100 |
| 3_125 | 99.5 | 98.4 | 100 | 100 | 100 | 100 |
| 236 | 100 | 100 | 100 | 100 | 100 | 100 |
| 2_236 | 100 | 100 | 100 | 100 | 100 | 100 |
| 1_236 | 100 | 100 | 100 | 100 | 100 | 100 |
| 237_236 | 99.5 | 98.4 | 100 | 99 | 96.8 | 100 |
| 4_236 | 100 | 100 | 100 | 99 | 96.8 | 100 |
| 3_236 | 100 | 100 | 100 | 100 | 100 | 100 |
| 46 | 95.4 | 95.4 | 95.4 | 95.4 | 95.4 | 95.4 |
| 2_46 | 99.5 | 98.4 | 100 | 100 | 100 | 100 |
| 1_46 | 99.5 | 98.4 | 100 | 100 | 100 | 100 |
| 237_46 | 99.5 | 98.4 | 100 | 98 | 93.5 | 100 |
| 4_46 | 100 | 100 | 100 | 99 | 96.8 | 100 |
| 3_46 | 100 | 100 | 100 | 100 | 100 | 100 |
| 32 | 97.5 | 97.5 | 97.5 | 97.5 | 97.5 | 97.5 |
| 2_32 | 99.5 | 98.4 | 100 | 100 | 100 | 100 |
| 1_32 | 100 | 100 | 100 | 100 | 100 | 100 |
| 237_32 | 99 | 96.8 | 100 | 99 | 96.8 | 100 |
| 4_32 | 99 | 96.8 | 100 | 98 | 93.5 | 100 |
| 3_32 | 100 | 100 | 100 | 100 | 100 | 100 |
| 62 | 95.4 | 95.4 | 95.4 | 95.4 | 95.4 | 95.4 |
| 2_62 | 99.5 | 98.4 | 100 | 100 | 100 | 100 |
| 162 | 99.5 | 98.4 | 100 | 100 | 100 | 100 |
| 237_62 | 99 | 96.7 | 100 | 99 | 96.8 | 100 |
| 4_62 | 99 | 96.7 | 100 | 98 | 93.5 | 100 |
| 3_62 | 100 | 100 | 100 | 99 | 96.8 | 100 |
| 88 | 94.9 | 94.9 | 94.9 | 94.9 | 94.9 | 94.9 |
| 2_88 | 100 | 100 | 100 | 100 | 100 | 100 |
| 1_88 | 99.5 | 98.4 | 100 | 100 | 100 | 100 |
| 237_88 | 100 | 100 | 100 | 99 | 96.8 | 100 |
| 4_88 | 100 | 100 | 100 | 100 | 100 | 100 |
| 3_88 | 100 | 100 | 100 | 100 | 100 | 100 |
| 52 | 95.4 | 95.4 | 95.4 | 95.4 | 95.4 | 95.4 |
| 2_52 | 100 | 100 | 100 | 100 | 100 | 100 |
| 1_52 | 100 | 100 | 100 | 100 | 100 | 100 |
| 237_52 | 100 | 100 | 100 | 99 | 96.8 | 100 |
| 4_52 | 99 | 96.8 | 100 | 99 | 96.8 | 100 |
| 3_52 | 100 | 100 | 100 | 100 | 100 | 100 |
| 7 | 98.5 | 98.5 | 98.5 | 98.5 | 98.5 | 98.5 |
| 2_7 | 99.5 | 98.4 | 100 | 100 | 100 | 100 |
| 1_7 | 99.5 | 98.4 | 100 | 100 | 100 | 100 |
| 237_7 | 99.5 | 98.4 | 100 | 100 | 100 | 100 |
| 4_7 | 99.5 | 98.4 | 100 | 99 | 96.8 | 100 |
| 3_7 | 100 | 100 | 100 | 100 | 100 | 100 |
| 26 | 94.4 | 94.4 | 94.4 | 94.4 | 94.4 | 94.4 |
| 2_26 | 99.5 | 98.4 | 100 | 100 | 100 | 100 |
| 1_26 | 100 | 100 | 100 | 100 | 100 | 100 |
| 237_26 | 99.5 | 98.4 | 100 | 99 | 96.8 | 100 |
| 4_26 | 100 | 100 | 100 | 99 | 96.8 | 100 |
| 3_26 | 100 | 100 | 100 | 100 | 100 | 100 |
| 25 | 99 | 99 | 99 | 99 | 99 | 99 |
| 2_25 | 99.5 | 98.4 | 100 | 100 | 100 | 100 |
| 1_25 | 100 | 100 | 100 | 100 | 100 | 100 |
| 237_25 | 99.5 | 98.4 | 100 | 100 | 100 | 100 |
| 4_25 | 100 | 100 | 100 | 100 | 100 | 100 |
| 3_25 | 100 | 100 | 100 | 100 | 100 | 100 |
| 54 | 95.9 | 95.9 | 95.9 | 95.9 | 95.9 | 95.9 |
| 2_54 | 100 | 100 | 100 | 100 | 100 | 100 |
| 1_54 | 99.5 | 98.4 | 100 | 100 | 100 | 100 |
| 237_54 | 99 | 96.8 | 100 | 99 | 96.8 | 100 |
| 4_54 | 99.5 | 98.4 | 100 | 99 | 96.8 | 100 |
| 3_54 | 99.5 | 98.4 | 100 | 100 | 100 | 100 |
| 92 | 94.4 | 94.4 | 94.4 | 94.4 | 94.4 | 94.4 |
| 2_92 | 100 | 100 | 100 | 100 | 100 | 100 |
| 1_92 | 99.5 | 98.4 | 100 | 100 | 100 | 100 |
| 237_92 | 99.5 | 98.4 | 100 | 98 | 93.5 | 100 |
| 4_92 | 99 | 96.8 | 100 | 99 | 96.8 | 100 |
| 3_92 | 99.5 | 98.4 | 100 | 100 | 100 | 100 |
| 14 | 96.4 | 96.4 | 96.4 | 96.4 | 96.4 | 96.4 |
| 2_14 | 100 | 100 | 100 | 100 | 100 | 100 |
| 1_14 | 100 | 100 | 100 | 100 | 100 | 100 |
| 237_14 | 100 | 100 | 100 | 100 | 100 | 100 |
| 4_14 | 100 | 100 | 100 | 100 | 100 | 100 |
| 3_14 | 100 | 100 | 100 | 100 | 100 | 100 |
| 242 | 95.4 | 95.4 | 95.4 | 95.4 | 95.4 | 95.4 |
| 2_242 | 99.5 | 98.4 | 100 | 100 | 100 | 100 |
| 1_242 | 100 | 100 | 100 | 100 | 100 | 100 |
| 237_242 | 98.5 | 95.2 | 100 | 99 | 96.8 | 100 |
| 4_242 | 99.5 | 98.4 | 100 | 99 | 96.8 | 100 |
| 3_242 | 99.5 | 98.4 | 100 | 100 | 100 | 100 |
| 47 | 97 | 97 | 97 | 97 | 97 | 97 |
| 2_47 | 99.5 | 98.4 | 100 | 100 | 100 | 100 |
| 1_47 | 100 | 100 | 100 | 100 | 100 | 100 |
| 237_47 | 98.5 | 95.2 | 100 | 98 | 93.5 | 100 |
| 4_47 | 99 | 96.8 | 100 | 98 | 93.5 | 100 |
| 3_47 | 100 | 100 | 100 | 100 | 100 | 100 |
| 45 | 95.9 | 95.9 | 95.9 | 95.9 | 95.9 | 95.9 |
| 2_45 | 100 | 100 | 100 | 100 | 100 | 100 |
| 1_45 | 100 | 100 | 100 | 100 | 100 | 100 |
| 237_45 | 99 | 96.8 | 100 | 98 | 93.5 | 100 |
| 4_45 | 100 | 100 | 100 | 99 | 96.8 | 100 |
| 3_45 | 100 | 100 | 100 | 100 | 100 | 100 |
| 39 | 98.5 | 98.5 | 98.5 | 98.5 | 98.5 | 98.5 |
| 2_39 | 99.5 | 98.4 | 100 | 100 | 100 | 100 |
| 1_39 | 99.5 | 98.4 | 100 | 100 | 100 | 100 |
| 237_39 | 99 | 96.8 | 100 | 100 | 100 | 100 |
| 4_39 | 99 | 96.8 | 100 | 100 | 100 | 100 |
| 3_39 | 100 | 100 | 100 | 100 | 100 | 100 |
| 21 | 95.9 | 95.9 | 95.9 | 95.9 | 95.9 | 95.9 |
| 2_21 | 100 | 100 | 100 | 100 | 100 | 100 |
| 1_21 | 99.5 | 98.4 | 100 | 100 | 100 | 100 |
| 237_21 | 99.5 | 100 | 99.3 | 100 | 100 | 100 |
| 4_21 | 99.5 | 100 | 99.3 | 100 | 100 | 100 |
| 3_21 | 99.5 | 98.4 | 100 | 100 | 100 | 100 |
| 17 | 98 | 98 | 98 | 98 | 98 | 98 |
| 2_17 | 99.5 | 98.4 | 100 | 100 | 100 | 100 |
| 1_17 | 99.5 | 98.4 | 100 | 100 | 100 | 100 |
| 237_17 | 99.5 | 98.4 | 100 | 100 | 100 | 100 |
| 4_17 | 99.5 | 98.4 | 100 | 100 | 100 | 100 |
| 3_17 | 99.5 | 98.4 | 100 | 100 | 100 | 100 |
| 83 | 96.4 | 96.4 | 96.4 | 96.4 | 96.4 | 96.4 |
| 2_83 | 99.5 | 98.4 | 100 | 100 | 100 | 100 |
| 1_83 | 99.5 | 98.4 | 100 | 100 | 100 | 100 |
| 237_83 | 99.5 | 98.4 | 100 | 99 | 96.8 | 100 |
| 4_83 | 100 | 100 | 100 | 100 | 100 | 100 |
| 3_83 | 99.5 | 98.4 | 100 | 100 | 100 | 100 |
| 149 | 94.9 | 94.9 | 94.9 | 94.9 | 94.9 | 94.9 |
| 2_149 | 99.5 | 98.4 | 100 | 100 | 100 | 100 |
| 1_149 | 99.5 | 98.4 | 100 | 100 | 100 | 100 |
| 237_149 | 99.5 | 98.4 | 100 | 99 | 96.8 | 100 |
| 4_149 | 99.5 | 98.4 | 100 | 99 | 96.8 | 100 |
| 3_149 | 99.5 | 98.4 | 100 | 100 | 100 | 100 |
| 246 | 94.4 | 94.4 | 94.4 | 94.4 | 94.4 | 94.4 |
| 2_246 | 99.5 | 98.4 | 100 | 100 | 100 | 100 |
| 1_246 | 99.5 | 98.4 | 100 | 100 | 100 | 100 |
| 237_246 | 98.5 | 95.2 | 100 | 98 | 93.5 | 100 |
| 4_246 | 99 | 96.8 | 100 | 98 | 93.5 | 100 |
| 3_246 | 100 | 100 | 100 | 100 | 100 | 100 |
| 22 | 94.9 | 94.9 | 94.9 | 94.9 | 94.9 | 94.9 |
| 2_22 | 100 | 100 | 100 | 100 | 100 | 100 |
| 1_22 | 99.5 | 98.4 | 100 | 100 | 100 | 100 |
| 237_22 | 99.5 | 98.4 | 100 | 100 | 100 | 100 |
| 4_22 | 99.5 | 98.4 | 100 | 99 | 96.8 | 100 |
| 3_22 | 100 | 100 | 100 | 100 | 100 | 100 |
| 55 | 98.5 | 98.5 | 98.5 | 98.5 | 98.5 | 98.5 |
| 2_55 | 99.5 | 98.4 | 100 | 100 | 100 | 100 |
| 1_55 | 100 | 100 | 100 | 100 | 100 | 100 |
| 237_55 | 99 | 96.8 | 100 | 100 | 100 | 100 |
| 4_55 | 99 | 96.8 | 100 | 100 | 100 | 100 |
| 3_55 | 100 | 100 | 100 | 100 | 100 | 100 |
| 1_82 | 97 | 97 | 97 | 97 | 97 | 97 |
| 2_182 | 99.5 | 98.4 | 100 | 100 | 100 | 100 |
| 1_182 | 99.5 | 98.4 | 100 | 100 | 100 | 100 |
| 237_182 | 100 | 100 | 100 | 100 | 100 | 100 |
| 4_182 | 100 | 100 | 100 | 100 | 100 | 100 |
| 3_182 | 100 | 100 | 100 | 100 | 100 | 100 |
| 73 | 98 | 98 | 98 | 98 | 98 | 98 |
| 2_73 | 99.5 | 98.4 | 100 | 100 | 100 | 100 |
| 1_73 | 100 | 100 | 100 | 100 | 100 | 100 |
| 237_73 | 99 | 96.8 | 100 | 99 | 96.8 | 100 |
| 4_73 | 99.5 | 98.4 | 100 | 99 | 96.8 | 100 |
| 3_73 | 100 | 100 | 100 | 100 | 100 | 100 |
| 77 | 93.9 | 93.9 | 93.9 | 93.9 | 93.9 | 93.9 |
| 2_77 | 99.5 | 98.4 | 100 | 100 | 100 | 100 |
| 1_77 | 99.5 | 98.4 | 100 | 100 | 100 | 100 |
| 237_77 | 99.5 | 98.4 | 100 | 99 | 96.8 | 100 |
| 4_77 | 99 | 96.8 | 100 | 98 | 93.5 | 100 |
| 3_77 | 100 | 100 | 100 | 100 | 100 | 100 |
| 24 | 94.4 | 94.4 | 94.4 | 94.4 | 94.4 | 94.4 |
| 2_24 | 100 | 100 | 100 | 100 | 100 | 100 |
| 1_24 | 99.5 | 98.4 | 100 | 100 | 100 | 100 |
| 237_24 | 100 | 100 | 100 | 100 | 100 | 100 |
| 4_24 | 100 | 100 | 100 | 100 | 100 | 100 |
| 3_24 | 99.5 | 98.4 | 100 | 99 | 96.8 | 100 |
| 103 | 92.4 | 92.4 | 92.4 | 92.4 | 92.4 | 92.4 |
| 2_103 | 100 | 100 | 100 | 100 | 100 | 100 |
| 1_103 | 100 | 100 | 100 | 100 | 100 | 100 |
| 237_103 | 99 | 96.8 | 100 | 98 | 93.5 | 100 |
| 4_103 | 99 | 96.8 | 100 | 99 | 96.8 | 100 |
| 3_103 | 100 | 100 | 100 | 100 | 100 | 100 |
| 49 | 94.4 | 94.4 | 94.4 | 94.4 | 94.4 | 94.4 |
| 2_49 | 100 | 100 | 100 | 100 | 100 | 100 |
| 1_49 | 100 | 100 | 100 | 100 | 100 | 100 |
| 237_49 | 99.5 | 98.4 | 100 | 98 | 93.5 | 100 |
| 4_49 | 100 | 100 | 100 | 99 | 96.8 | 100 |
| 3_49 | 100 | 100 | 100 | 100 | 100 | 100 |
| 239 | 98 | 98 | 98 | 98 | 98 | 98 |
| 2_239 | 100 | 100 | 100 | 100 | 100 | 100 |
| 1_239 | 100 | 100 | 100 | 100 | 100 | 100 |
| 237_239 | 100 | 100 | 100 | 100 | 100 | 100 |
| 4_239 | 100 | 100 | 100 | 100 | 100 | 100 |
| 3_239 | 100 | 100 | 100 | 100 | 100 | 100 |
| 23 | 89.3 | 89.3 | 89.3 | 89.3 | 89.3 | 89.3 |
| 2_23 | 100 | 100 | 100 | 100 | 100 | 100 |
| 1_23 | 100 | 100 | 100 | 100 | 100 | 100 |
| 237_23 | 99.5 | 98.4 | 100 | 100 | 100 | 100 |
| 4_23 | 100 | 100 | 100 | 100 | 100 | 100 |
| 3_23 | 100 | 100 | 100 | 100 | 100 | 100 |
| 58 | 94.4 | 94.4 | 94.4 | 94.4 | 94.4 | 94.4 |
| 2_58 | 99.5 | 98.4 | 100 | 100 | 100 | 100 |
| 1_58 | 100 | 100 | 100 | 100 | 100 | 100 |
| 237_58 | 100 | 100 | 100 | 99 | 96.8 | 100 |
| 4_58 | 100 | 100 | 100 | 99 | 96.8 | 100 |
| 3_58 | 100 | 100 | 100 | 100 | 100 | 100 |
| 211 | 93.4 | 93.4 | 93.4 | 93.4 | 93.4 | 93.4 |
| 2_211 | 99.5 | 98.4 | 100 | 100 | 100 | 100 |
| 1_211 | 99.5 | 98.4 | 100 | 100 | 100 | 100 |
| 237_211 | 100 | 100 | 100 | 96.9 | 90.3 | 100 |
| 4_211 | 99.5 | 98.4 | 100 | 99 | 96.8 | 100 |
| 3_211 | 99.5 | 98.4 | 100 | 100 | 100 | 100 |
| 147 | 93.4 | 93.4 | 93.4 | 93.4 | 93.4 | 93.4 |
| 2_147 | 100 | 100 | 100 | 100 | 100 | 100 |
| 1_147 | 99.5 | 98.4 | 100 | 100 | 100 | 100 |
| 237_147 | 100 | 100 | 100 | 100 | 100 | 100 |
| 4_147 | 99.5 | 98.4 | 100 | 100 | 100 | 100 |
| 3_147 | 99.5 | 98.4 | 100 | 100 | 100 | 100 |
| 65 | 90.9 | 90.9 | 90.9 | 90.9 | 90.9 | 90.9 |
| 2_65 | 99.5 | 98.4 | 100 | 100 | 100 | 100 |
| 1_65 | 99.5 | 98.4 | 100 | 100 | 100 | 100 |
| 237_65 | 99 | 96.8 | 100 | 98 | 93.5 | 100 |
| 4_65 | 99 | 96.8 | 100 | 98 | 93.5 | 100 |
| 3_65 | 99.5 | 98.4 | 100 | 100 | 100 | 100 |
| 31 | 91.9 | 91.9 | 91.9 | 91.9 | 91.9 | 91.9 |
| 2_31 | 99.5 | 98.4 | 100 | 100 | 100 | 100 |
| 1_31 | 99.5 | 98.4 | 100 | 100 | 100 | 100 |
| 237_31 | 99.5 | 98.4 | 100 | 99 | 96.8 | 100 |
| 4_31 | 99 | 96.8 | 100 | 99 | 96.8 | 100 |
| 3_31 | 99.5 | 98.4 | 100 | 100 | 100 | 100 |
| 72 | 90.4 | 90.4 | 90.4 | 90.4 | 90.4 | 90.4 |
| 2_72 | 99.5 | 98.4 | 100 | 100 | 100 | 100 |
| 1_72 | 100 | 100 | 100 | 100 | 100 | 100 |
| 237_72 | 98.5 | 95.2 | 100 | 100 | 100 | 100 |
| 4_72 | 99.5 | 98.4 | 100 | 100 | 100 | 100 |
| 3_72 | 100 | 100 | 100 | 100 | 100 | 100 |
| 63 | 87.8 | 87.8 | 87.8 | 87.8 | 87.8 | 87.8 |
| 2_63 | 100 | 100 | 100 | 100 | 100 | 100 |
| 1_63 | 99.5 | 98.4 | 100 | 100 | 100 | 100 |
| 237_63 | 99.5 | 98.4 | 100 | 98 | 93.5 | 100 |
| 4_63 | 99.5 | 98.4 | 100 | 99 | 96.8 | 100 |
| 3_63 | 99.5 | 98.4 | 100 | 100 | 100 | 100 |
| 80 | 88.3 | 88.3 | 88.3 | 88.3 | 88.3 | 88.3 |
| 2_80 | 100 | 100 | 100 | 100 | 100 | 100 |
| 1_80 | 99.5 | 98.4 | 100 | 100 | 100 | 100 |
| 237_80 | 100 | 100 | 100 | 100 | 100 | 100 |
| 4_80 | 100 | 100 | 100 | 99 | 96.8 | 100 |
| 3_80 | 99.5 | 98.4 | 100 | 100 | 100 | 100 |
| 37 | 97 | 97 | 97 | 97 | 97 | 97 |
| 2_37 | 99.5 | 98.4 | 100 | 100 | 100 | 100 |
| 1_37 | 99.5 | 98.4 | 100 | 100 | 100 | 100 |
| 237_37 | 99.5 | 98.4 | 100 | 100 | 100 | 100 |
| 4_37 | 99.5 | 98.4 | 100 | 99 | 96.8 | 100 |
| 3_37 | 100 | 100 | 100 | 100 | 100 | 100 |
| 67 | 92.4 | 92.4 | 92.4 | 92.4 | 92.4 | 92.4 |
| 2_67 | 99.5 | 98.4 | 100 | 100 | 100 | 100 |
| 1_67 | 100 | 100 | 100 | 100 | 100 | 100 |
| 237_67 | 98.5 | 95.2 | 100 | 99 | 96.8 | 100 |
| 4_67 | 99 | 96.8 | 100 | 99 | 96.8 | 100 |
| 3_67 | 100 | 100 | 100 | 100 | 100 | 100 |
| 232 | 90.9 | 90.9 | 90.9 | 90.9 | 90.9 | 90.9 |
| 2_232 | 99.5 | 98.4 | 100 | 100 | 100 | 100 |
| 1_232 | 99.5 | 98.4 | 100 | 100 | 100 | 100 |
| 237_232 | 99.5 | 98.4 | 100 | 98 | 93.5 | 100 |
| 4_232 | 99 | 96.8 | 100 | 99 | 96.8 | 100 |
| 3_232 | 99.5 | 98.4 | 100 | 100 | 100 | 100 |
| 127 | 83.8 | 83.8 | 83.8 | 83.8 | 83.8 | 83.8 |
| 2_127 | 99.5 | 98.4 | 100 | 100 | 100 | 100 |
| 1_127 | 100 | 100 | 100 | 100 | 100 | 100 |
| 237_127 | 98.5 | 95.2 | 100 | 98 | 93.5 | 100 |
| 4_127 | 99 | 96.8 | 100 | 98 | 93.5 | 100 |
| 3_127 | 100 | 100 | 100 | 99 | 96.8 | 100 |
| 145 | 91.9 | 91.9 | 91.9 | 91.9 | 91.9 | 91.9 |
| 2_145 | 100 | 100 | 100 | 100 | 100 | 100 |
| 1_145 | 99.5 | 98.4 | 100 | 100 | 100 | 100 |
| 237_145 | 100 | 100 | 100 | 99 | 96.8 | 100 |
| 4_145 | 99.5 | 98.4 | 100 | 100 | 100 | 100 |
| 3_145 | 100 | 100 | 100 | 100 | 100 | 100 |
| 16 | 98.5 | 98.5 | 98.5 | 98.5 | 98.5 | 98.5 |
| 2_16 | 100 | 100 | 100 | 100 | 100 | 100 |
| 1_16 | 99.5 | 98.4 | 100 | 100 | 100 | 100 |
| 237_16 | 100 | 100 | 100 | 100 | 100 | 100 |
| 4_16 | 100 | 100 | 100 | 99 | 96.8 | 100 |
| 3_16 | 99.5 | 98.4 | 100 | 99 | 96.8 | 100 |
| 11 | 99.5 | 99.5 | 99.5 | 99.5 | 99.5 | 99.5 |
| 2_11 | 100 | 100 | 100 | 100 | 100 | 100 |
| 1_11 | 99.5 | 98.4 | 100 | 100 | 100 | 100 |
| 237_11 | 100 | 100 | 100 | 100 | 100 | 100 |
| 4_11 | 100 | 100 | 100 | 100 | 100 | 100 |
| 3_11 | 100 | 100 | 100 | 100 | 100 | 100 |
| 186 | 87.3 | 87.3 | 87.3 | 87.3 | 87.3 | 87.3 |
| 2_186 | 99.5 | 98.4 | 100 | 100 | 100 | 100 |
| 1_186 | 99.5 | 98.4 | 100 | 100 | 100 | 100 |
| 237_186 | 98.5 | 95.2 | 100 | 96.9 | 90.3 | 100 |
| 4_186 | 99.5 | 98.4 | 100 | 99 | 96.8 | 100 |
| 3_186 | 100 | 100 | 100 | 100 | 100 | 100 |
| 50 | 98.5 | 98.5 | 98.5 | 98.5 | 98.5 | 98.5 |
| 2_50 | 100 | 100 | 100 | 100 | 100 | 100 |
| 1_50 | 99.5 | 98.4 | 100 | 100 | 100 | 100 |
| 237_50 | 100 | 100 | 100 | 100 | 100 | 100 |
| 4_50 | 100 | 100 | 100 | 100 | 100 | 100 |
| 3_50 | 100 | 100 | 100 | 100 | 100 | 100 |
| 69 | 90.9 | 90.9 | 90.9 | 90.9 | 90.9 | 90.9 |
| 2_69 | 99.5 | 98.4 | 100 | 100 | 100 | 100 |
| 1_69 | 99.5 | 98.4 | 100 | 100 | 100 | 100 |
| 237_69 | 98.5 | 95.2 | 100 | 98 | 93.5 | 100 |
| 4_69 | 99 | 96.8 | 100 | 98 | 93.5 | 100 |
| 3_69 | 100 | 100 | 100 | 100 | 100 | 100 |
| 33 | 81.2 | 81.2 | 81.2 | 81.2 | 81.2 | 81.2 |
| 2_33 | 100 | 100 | 100 | 100 | 100 | 100 |
| 1_33 | 99.5 | 98.4 | 100 | 100 | 100 | 100 |
| 237_33 | 100 | 100 | 100 | 100 | 100 | 100 |
| 4_33 | 100 | 100 | 100 | 100 | 100 | 100 |
| 3_33 | 99.5 | 98.4 | 100 | 100 | 100 | 100 |
| 247 | 89.3 | 89.3 | 89.3 | 89.3 | 89.3 | 89.3 |
| 2_247 | 100 | 100 | 100 | 100 | 100 | 100 |
| 1_247 | 100 | 100 | 100 | 100 | 100 | 100 |
| 237_247 | 98.5 | 95.2 | 100 | 98 | 93.5 | 100 |
| 4_247 | 99 | 96.8 | 100 | 99 | 96.8 | 100 |
| 3_247 | 100 | 100 | 100 | 100 | 100 | 100 |
| 36 | 93.9 | 93.9 | 93.9 | 93.9 | 93.9 | 93.9 |
| 2_36 | 99.5 | 98.4 | 100 | 100 | 100 | 100 |
| 1_36 | 99.5 | 98.4 | 100 | 100 | 100 | 100 |
| 237_36 | 100 | 100 | 100 | 99 | 96.8 | 100 |
| 4_36 | 99 | 96.8 | 100 | 98 | 93.5 | 100 |
| 3_36 | 100 | 100 | 100 | 100 | 100 | 100 |
| 218 | 89.3 | 89.3 | 89.3 | 89.3 | 89.3 | 89.3 |
| 2_218 | 99.5 | 98.4 | 100 | 100 | 100 | 100 |
| 1_218 | 99.5 | 98.4 | 100 | 100 | 100 | 100 |
| 237_218 | 99 | 96.8 | 100 | 100 | 100 | 100 |
| 4_218 | 99 | 96.8 | 100 | 99 | 96.8 | 100 |
| 3_218 | 100 | 100 | 100 | 100 | 100 | 100 |
| 43 | 82.7 | 82.7 | 82.7 | 82.7 | 82.7 | 82.7 |
| 2_43 | 99.5 | 98.4 | 100 | 100 | 100 | 100 |
| 1_43 | 99.5 | 98.4 | 100 | 100 | 100 | 100 |
| 237_43 | 100 | 100 | 100 | 99 | 96.8 | 100 |
| 4_43 | 99.5 | 98.4 | 100 | 99 | 96.8 | 100 |
| 3_43 | 99.5 | 98.4 | 100 | 100 | 100 | 100 |
| 29 | 96.4 | 96.4 | 96.4 | 96.4 | 96.4 | 96.4 |
| 2_29 | 99.5 | 98.4 | 100 | 100 | 100 | 100 |
| 1_29 | 99.5 | 98.4 | 100 | 100 | 100 | 100 |
| 237_29 | 98.5 | 95.2 | 100 | 100 | 100 | 100 |
| 4_29 | 99.5 | 98.4 | 100 | 100 | 100 | 100 |
| 3_29 | 100 | 100 | 100 | 100 | 100 | 100 |
| 110 | 83.8 | 83.8 | 83.8 | 83.8 | 83.8 | 83.8 |
| 2_110 | 99.5 | 98.4 | 100 | 100 | 100 | 100 |
| 1_110 | 99.5 | 98.4 | 100 | 100 | 100 | 100 |
| 237_110 | 99 | 96.8 | 100 | 96.9 | 90.3 | 100 |
| 4_110 | 99 | 96.8 | 100 | 99 | 96.8 | 100 |
| 3_110 | 99.5 | 98.4 | 100 | 99 | 96.8 | 100 |
| 20 | 95.9 | 95.9 | 95.9 | 95.9 | 95.9 | 95.9 |
| 2_20 | 100 | 100 | 100 | 100 | 100 | 100 |
| 1_20 | 100 | 100 | 100 | 100 | 100 | 100 |
| 237_20 | 99 | 96.7 | 100 | 99 | 96.8 | 100 |
| 4_20 | 99.5 | 98.4 | 100 | 98 | 93.5 | 100 |
| 3_20 | 100 | 100 | 100 | 100 | 100 | 100 |
| 157 | 89.3 | 89.3 | 89.3 | 89.3 | 89.3 | 89.3 |
| 2_157 | 100 | 100 | 100 | 100 | 100 | 100 |
| 1_157 | 99.5 | 98.4 | 100 | 100 | 100 | 100 |
| 237_157 | 100 | 100 | 100 | 98 | 93.5 | 100 |
| 4_157 | 99 | 96.8 | 100 | 99 | 96.8 | 100 |
| 3_157 | 100 | 100 | 100 | 100 | 100 | 100 |
| 75 | 90.9 | 90.9 | 90.9 | 90.9 | 90.9 | 90.9 |
| 2_75 | 100 | 100 | 100 | 100 | 100 | 100 |
| 1_75 | 99.5 | 98.4 | 100 | 100 | 100 | 100 |
| 237_75 | 99.5 | 98.4 | 100 | 100 | 100 | 100 |
| 4_75 | 100 | 100 | 100 | 100 | 100 | 100 |
| 3_75 | 99.5 | 98.4 | 100 | 100 | 100 | 100 |
| 82 | 89.3 | 89.3 | 89.3 | 89.3 | 89.3 | 89.3 |
| 2_82 | 100 | 100 | 100 | 100 | 100 | 100 |
| 1_82 | 99.5 | 98.4 | 100 | 100 | 100 | 100 |
| 237_82 | 99 | 96.8 | 100 | 98 | 93.5 | 100 |
| 4_82 | 100 | 100 | 100 | 99 | 96.8 | 100 |
| 3_82 | 100 | 100 | 100 | 100 | 100 | 100 |
| 106 | 89.3 | 89.3 | 89.3 | 89.3 | 89.3 | 89.3 |
| 2_106 | 99.5 | 98.4 | 100 | 100 | 100 | 100 |
| 1_106 | 100 | 100 | 100 | 100 | 100 | 100 |
| 237_106 | 99 | 96.8 | 100 | 99 | 96.8 | 100 |
| 4_106 | 99.5 | 98.4 | 100 | 100 | 100 | 100 |
| 3_106 | 100 | 100 | 100 | 100 | 100 | 100 |
| 111 | 81.7 | 81.7 | 81.7 | 81.7 | 81.7 | 81.7 |
| 2_111 | 99.5 | 98.4 | 100 | 100 | 100 | 100 |
| 1_111 | 99.5 | 98.4 | 100 | 100 | 100 | 100 |
| 237_111 | 98.5 | 95.2 | 100 | 98 | 93.5 | 100 |
| 4_111 | 99 | 96.8 | 100 | 99 | 96.8 | 100 |
| 3_111 | 99.5 | 98.4 | 100 | 100 | 100 | 100 |
| 96 | 86.3 | 86.3 | 86.3 | 86.3 | 86.3 | 86.3 |
| 2_96 | 99.5 | 98.4 | 100 | 100 | 100 | 100 |
| 1_96 | 99.5 | 98.4 | 100 | 100 | 100 | 100 |
| 237_96 | 99 | 96.8 | 100 | 99 | 96.8 | 100 |
| 4_96 | 99 | 96.8 | 100 | 99 | 96.8 | 100 |
| 3_96 | 99.5 | 98.4 | 100 | 100 | 100 | 100 |
| 266 | 87.8 | 87.8 | 87.8 | 87.8 | 87.8 | 87.8 |
| 2_266 | 99.5 | 98.4 | 100 | 100 | 100 | 100 |
| 1_266 | 99.5 | 98.4 | 100 | 100 | 100 | 100 |
| 237_266 | 99 | 96.8 | 100 | 98 | 93.5 | 100 |
| 4_266 | 99.5 | 98.4 | 100 | 99 | 96.8 | 100 |
| 3_266 | 99.5 | 98.4 | 100 | 99 | 96.8 | 100 |
| 124 | 91.9 | 91.9 | 91.9 | 91.9 | 91.9 | 91.9 |
| 2_124 | 100 | 100 | 100 | 100 | 100 | 100 |
| 1_124 | 99.5 | 98.4 | 100 | 100 | 100 | 100 |
| 237_124 | 100 | 100 | 100 | 100 | 100 | 100 |
| 4_124 | 100 | 100 | 100 | 100 | 100 | 100 |
| 3_124 | 99.5 | 98.4 | 100 | 100 | 100 | 100 |
| 68 | 81.2 | 81.2 | 81.2 | 81.2 | 81.2 | 81.2 |
| 2_68 | 99.5 | 98.4 | 100 | 100 | 100 | 100 |
| 1_68 | 99.5 | 98.4 | 100 | 100 | 100 | 100 |
| 237_68 | 99 | 96.8 | 100 | 99 | 96.8 | 100 |
| 4_68 | 99 | 96.8 | 100 | 99 | 96.8 | 100 |
| 3_68 | 99.5 | 98.4 | 100 | 100 | 100 | 100 |
| 71 | 86.8 | 86.8 | 86.8 | 86.8 | 86.8 | 86.8 |
| 2_71 | 100 | 100 | 100 | 100 | 100 | 100 |
| 1_71 | 99.5 | 98.4 | 100 | 100 | 100 | 100 |
| 237_71 | 99 | 96.8 | 100 | 96.9 | 90.3 | 100 |
| 4_71 | 99 | 96.8 | 100 | 99 | 96.8 | 100 |
| 3_71 | 100 | 100 | 100 | 99 | 96.8 | 100 |
| 35 | 90.4 | 90.4 | 90.4 | 90.4 | 90.4 | 90.4 |
| 2_35 | 99.5 | 98.4 | 100 | 100 | 100 | 100 |
| 1_35 | 99.5 | 98.4 | 100 | 100 | 100 | 100 |
| 237_35 | 99.5 | 98.4 | 100 | 100 | 100 | 100 |
| 4_35 | 99.5 | 98.4 | 100 | 100 | 100 | 100 |
| 3_35 | 99.5 | 98.4 | 100 | 100 | 100 | 100 |
| 173 | 82.2 | 82.2 | 82.2 | 82.2 | 82.2 | 82.2 |
| 2_173 | 99.5 | 98.4 | 100 | 100 | 100 | 100 |
| 1_173 | 99.5 | 98.4 | 100 | 100 | 100 | 100 |
| 237_173 | 98.5 | 95.2 | 100 | 98 | 93.5 | 100 |
| 4_173 | 99 | 96.8 | 100 | 98 | 93.5 | 100 |
| 3_173 | 99.5 | 98.4 | 100 | 100 | 100 | 100 |
| 5 | 94.4 | 94.4 | 94.4 | 94.4 | 94.4 | 94.4 |
| 2_5 | 100 | 100 | 100 | 100 | 100 | 100 |
| 1_5 | 99.5 | 98.4 | 100 | 100 | 100 | 100 |
| 237_5 | 100 | 100 | 100 | 100 | 100 | 100 |
| 4_5 | 100 | 100 | 100 | 100 | 100 | 100 |
| 3_5 | 99.5 | 98.4 | 100 | 100 | 100 | 100 |
| 851 | 81.7 | 81.7 | 81.7 | 81.7 | 81.7 | 81.7 |
| 2_851 | 99.5 | 98.4 | 100 | 100 | 100 | 100 |
| 1_851 | 99.5 | 98.4 | 100 | 100 | 100 | 100 |
| 237_851 | 99 | 96.8 | 100 | 98 | 93.5 | 100 |
| 4_851 | 99 | 96.8 | 100 | 99 | 96.8 | 100 |
| 3_851 | 99.5 | 98.4 | 100 | 100 | 100 | 100 |
| 852 | 85.8 | 85.8 | 85.8 | 85.8 | 85.8 | 85.8 |
| 2_852 | 100 | 100 | 100 | 100 | 100 | 100 |
| 1_852 | 99.5 | 98.4 | 100 | 100 | 100 | 100 |
| 237_852 | 99 | 96.8 | 100 | 98 | 93.5 | 100 |
| 4_852 | 99 | 96.8 | 100 | 99 | 96.8 | 100 |
| 3_852 | 100 | 100 | 100 | 100 | 100 | 100 |
| 30 | 93.4 | 93.4 | 93.4 | 93.4 | 93.4 | 93.4 |
| 2_30 | 100 | 100 | 100 | 100 | 100 | 100 |
| 1_30 | 99.5 | 98.4 | 100 | 100 | 100 | 100 |
| 237_30 | 99 | 96.8 | 100 | 100 | 100 | 100 |
| 4_30 | 99.5 | 98.4 | 100 | 99 | 96.8 | 100 |
| 3_30 | 99.5 | 98.4 | 100 | 100 | 100 | 100 |
| 93 | 88.8 | 88.8 | 88.8 | 88.8 | 88.8 | 88.8 |
| 2_93 | 99.5 | 98.4 | 100 | 100 | 100 | 100 |
| 1_93 | 99.5 | 98.4 | 100 | 100 | 100 | 100 |
| 237_93 | 99.5 | 98.4 | 100 | 100 | 100 | 100 |
| 4_93 | 100 | 100 | 100 | 100 | 100 | 100 |
| 3_93 | 99.5 | 98.4 | 100 | 100 | 100 | 100 |
| 27 | 98.5 | 98.5 | 98.5 | 98.5 | 98.5 | 98.5 |
| 2_27 | 100 | 100 | 100 | 100 | 100 | 100 |
| 1_27 | 99.5 | 98.4 | 100 | 100 | 100 | 100 |
| 237_27 | 100 | 100 | 100 | 100 | 100 | 100 |
| 4_27 | 99.5 | 98.4 | 100 | 99 | 96.8 | 100 |
| 27_208 | 99 | 98.4 | 99.3 | 98 | 96.8 | 98.5 |
| 853 | 79.2 | 79.2 | 79.2 | 79.2 | 79.2 | 79.2 |
| 2_853 | 99.5 | 98.4 | 100 | 100 | 100 | 100 |
| 1_853 | 99.5 | 98.4 | 100 | 100 | 100 | 100 |
| 237_853 | 98.5 | 95.2 | 100 | 98 | 93.5 | 100 |
| 4_853 | 99 | 96.8 | 100 | 99 | 96.8 | 100 |
| 3_853 | 99.5 | 98.4 | 100 | 100 | 100 | 100 |
| 238 | 97.5 | 97.5 | 97.5 | 97.5 | 97.5 | 97.5 |
| 2_238 | 100 | 100 | 100 | 100 | 100 | 100 |
| 1_238 | 100 | 100 | 100 | 100 | 100 | 100 |
| 237_238 | 99 | 96.8 | 100 | 99 | 96.8 | 100 |
| 4_238 | 99 | 96.8 | 100 | 99 | 96.8 | 100 |
| 3_238 | 100 | 100 | 100 | 100 | 100 | 100 |
| 130 | 80.7 | 80.7 | 80.7 | 80.7 | 80.7 | 80.7 |
| 2_130 | 100 | 100 | 100 | 100 | 100 | 100 |
| 1_130 | 99.5 | 98.4 | 100 | 100 | 100 | 100 |
| 237_130 | 99 | 96.8 | 100 | 99 | 96.8 | 100 |
| 4_130 | 99 | 96.8 | 100 | 99 | 96.8 | 100 |
| 3_130 | 99.5 | 98.4 | 100 | 100 | 100 | 100 |
| 177 | 81.7 | 81.7 | 81.7 | 81.7 | 81.7 | 81.7 |
| 2_177 | 100 | 100 | 100 | 100 | 100 | 100 |
| 1_177 | 99.5 | 98.4 | 100 | 100 | 100 | 100 |
| 237_177 | 99 | 96.8 | 100 | 99 | 96.8 | 100 |
| 4_177 | 99.5 | 98.4 | 100 | 99 | 96.8 | 100 |
| 3_177 | 99.5 | 98.4 | 100 | 99 | 96.8 | 100 |
| 64 | 87.3 | 87.3 | 87.3 | 87.3 | 87.3 | 87.3 |
| 2_64 | 99.5 | 98.4 | 100 | 100 | 100 | 100 |
| 1_64 | 99.5 | 98.4 | 100 | 100 | 100 | 100 |
| 237_64 | 99.5 | 98.4 | 100 | 100 | 100 | 100 |
| 4_64 | 99.5 | 98.4 | 100 | 100 | 100 | 100 |
| 3_64 | 99.5 | 98.4 | 100 | 100 | 100 | 100 |
| 114 | 95.4 | 95.4 | 95.4 | 95.4 | 95.4 | 95.4 |
| 2_114 | 100 | 100 | 100 | 99 | 100 | 98.5 |
| 1_114 | 99.5 | 98.4 | 100 | 99 | 100 | 98.5 |
| 237_114 | 99.5 | 98.4 | 100 | 99 | 100 | 98.5 |
| 4_114 | 100 | 100 | 100 | 99 | 100 | 98.5 |
| 3_114 | 99.5 | 98.4 | 100 | 99 | 100 | 98.5 |
| 119 | 92.4 | 92.4 | 92.4 | 92.4 | 92.4 | 92.4 |
| 2_119 | 100 | 100 | 100 | 100 | 100 | 100 |
| 1_119 | 99.5 | 98.4 | 100 | 100 | 100 | 100 |
| 237_119 | 100 | 100 | 100 | 99 | 96.8 | 100 |
| 4_119 | 100 | 100 | 100 | 100 | 100 | 100 |
| 3_119 | 99.5 | 98.4 | 100 | 100 | 100 | 100 |
| 135 | 87.8 | 87.8 | 87.8 | 87.8 | 87.8 | 87.8 |
| 2_135 | 99.5 | 98.4 | 100 | 100 | 100 | 100 |
| 1_135 | 99.5 | 98.4 | 100 | 100 | 100 | 100 |
| 237_135 | 99.5 | 98.4 | 100 | 96.9 | 90.3 | 100 |
| 4_135 | 99.5 | 98.4 | 100 | 99 | 96.8 | 100 |
| 3_135 | 99.5 | 98.4 | 100 | 99 | 96.8 | 100 |
| 243 | 93.9 | 93.9 | 93.9 | 93.9 | 93.9 | 93.9 |
| 2_243 | 99.5 | 98.4 | 100 | 100 | 100 | 100 |
| 1_243 | 99.5 | 98.4 | 100 | 100 | 100 | 100 |
| 237_243 | 99.5 | 98.4 | 100 | 99 | 96.8 | 100 |
| 4_243 | 99.5 | 98.4 | 100 | 100 | 100 | 100 |
| 3_243 | 100 | 100 | 100 | 100 | 100 | 100 |
| 122 | 87.8 | 87.8 | 87.8 | 87.8 | 87.8 | 87.8 |
| 2_122 | 99.5 | 98.4 | 100 | 100 | 100 | 100 |
| 1_122 | 99.5 | 98.4 | 100 | 100 | 100 | 100 |
| 237_122 | 98.5 | 95.2 | 100 | 96.9 | 90.3 | 100 |
| 4_122 | 99 | 96.8 | 100 | 99 | 96.8 | 100 |
| 3_122 | 100 | 100 | 100 | 100 | 100 | 100 |
| 260 | 90.9 | 90.9 | 90.9 | 90.9 | 90.9 | 90.9 |
| 2_260 | 99.5 | 98.4 | 100 | 100 | 100 | 100 |
| 1_260 | 99.5 | 98.4 | 100 | 100 | 100 | 100 |
| 237_260 | 99.5 | 98.4 | 100 | 96.9 | 90.3 | 100 |
| 4_260 | 99.5 | 98.4 | 100 | 99 | 96.8 | 100 |
| 3_260 | 99.5 | 98.4 | 100 | 99 | 96.8 | 100 |
| 59 | 91.9 | 91.9 | 91.9 | 91.9 | 91.9 | 91.9 |
| 2_59 | 99.5 | 98.4 | 100 | 100 | 100 | 100 |
| 1_59 | 99.5 | 98.4 | 100 | 100 | 100 | 100 |
| 237_59 | 99.5 | 98.4 | 100 | 100 | 100 | 100 |
| 4_59 | 99 | 96.8 | 100 | 99 | 96.8 | 100 |
| 3_59 | 99.5 | 98.4 | 100 | 100 | 100 | 100 |
| 854 | 77.7 | 77.7 | 77.7 | 77.7 | 77.7 | 77.7 |
| 2_854 | 99.5 | 98.4 | 100 | 100 | 100 | 100 |
| 1_854 | 99.5 | 98.4 | 100 | 100 | 100 | 100 |
| 237_854 | 99.5 | 98.4 | 100 | 99 | 96.8 | 100 |
| 4_854 | 99 | 96.8 | 100 | 99 | 96.8 | 100 |
| 3_854 | 99.5 | 98.4 | 100 | 100 | 100 | 100 |
| 132 | 91.4 | 91.4 | 91.4 | 91.4 | 91.4 | 91.4 |
| 2_132 | 100 | 100 | 100 | 100 | 100 | 100 |
| 1_132 | 99.5 | 98.4 | 100 | 100 | 100 | 100 |
| 237_132 | 99.5 | 98.4 | 100 | 98 | 93.5 | 100 |
| 4_132 | 99.5 | 98.4 | 100 | 99 | 96.8 | 100 |
| 3_132 | 99.5 | 98.4 | 100 | 99 | 96.8 | 100 |
| 181 | 79.2 | 79.2 | 79.2 | 79.2 | 79.2 | 79.2 |
| 2_181 | 99.5 | 98.4 | 100 | 100 | 100 | 100 |
| 1_181 | 99.5 | 98.4 | 100 | 100 | 100 | 100 |
| 237181 | 99 | 96.8 | 100 | 99 | 96.8 | 100 |
| 4_181 | 99 | 96.8 | 100 | 98 | 93.5 | 100 |
| 3_181 | 99.5 | 98.4 | 100 | 100 | 100 | 100 |
| 79 | 92.4 | 92.4 | 92.4 | 92.4 | 92.4 | 92.4 |
| 2_79 | 100 | 100 | 100 | 100 | 100 | 100 |
| 1_79 | 99.5 | 98.4 | 100 | 100 | 100 | 100 |
| 237_79 | 99.5 | 98.4 | 100 | 98 | 93.5 | 100 |
| 4_79 | 99.5 | 98.4 | 100 | 99 | 96.8 | 100 |
| 3_79 | 99.5 | 98.4 | 100 | 100 | 100 | 100 |
| 133 | 79.7 | 79.7 | 79.7 | 79.7 | 79.7 | 79.7 |
| 2_133 | 99.5 | 98.4 | 100 | 100 | 100 | 100 |
| 1_133 | 99.5 | 98.4 | 100 | 100 | 100 | 100 |
| 237_133 | 98.5 | 95.2 | 100 | 99 | 96.8 | 100 |
| 4_133 | 99 | 96.8 | 100 | 99 | 96.8 | 100 |
| 3_133 | 99.5 | 98.4 | 100 | 100 | 100 | 100 |
| 41 | 91.4 | 91.4 | 91.4 | 91.4 | 91.4 | 91.4 |
| 2_41 | 99.5 | 98.4 | 100 | 100 | 100 | 100 |
| 1_41 | 99.5 | 98.4 | 100 | 100 | 100 | 100 |
| 237_41 | 99 | 96.8 | 100 | 99 | 96.8 | 100 |
| 4_41 | 99.5 | 98.4 | 100 | 99 | 96.8 | 100 |
| 3_41 | 99.5 | 98.4 | 100 | 100 | 100 | 100 |
| 139 | 88.3 | 88.3 | 88.3 | 88.3 | 88.3 | 88.3 |
| 2_139 | 100 | 100 | 100 | 100 | 100 | 100 |
| 1_139 | 99.5 | 98.4 | 100 | 100 | 100 | 100 |
| 237_139 | 100 | 100 | 100 | 98 | 93.5 | 100 |
| 4_139 | 100 | 100 | 100 | 99 | 96.8 | 100 |
| 3_139 | 99.5 | 98.4 | 100 | 100 | 100 | 100 |
| 118 | 87.8 | 87.8 | 87.8 | 87.8 | 87.8 | 87.8 |
| 2_118 | 99.5 | 98.4 | 100 | 100 | 100 | 100 |
| 1_118 | 99.5 | 98.4 | 100 | 100 | 100 | 100 |
| 237_118 | 99 | 96.8 | 100 | 100 | 100 | 100 |
| 4_118 | 99.5 | 98.4 | 100 | 100 | 100 | 100 |
| 3_118 | 99.5 | 98.4 | 100 | 100 | 100 | 100 |
| 86 | 85.3 | 85.3 | 85.3 | 85.3 | 85.3 | 85.3 |
| 2_86 | 99.5 | 98.4 | 100 | 100 | 100 | 100 |
| 1_86 | 99.5 | 98.4 | 100 | 100 | 100 | 100 |
| 237_86 | 99 | 96.8 | 100 | 100 | 100 | 100 |
| 4_86 | 99 | 96.8 | 100 | 100 | 100 | 100 |
| 3_86 | 99.5 | 98.4 | 100 | 99 | 96.8 | 100 |
| 60 | 84.8 | 84.8 | 84.8 | 84.8 | 84.8 | 84.8 |
| 2_60 | 99.5 | 98.4 | 100 | 100 | 100 | 100 |
| 1_60 | 99.5 | 98.4 | 100 | 100 | 100 | 100 |
| 237_60 | 99 | 96.8 | 100 | 99 | 96.8 | 100 |
| 4_60 | 99.5 | 98.4 | 100 | 99 | 96.8 | 100 |
| 3_60 | 99.5 | 98.4 | 100 | 100 | 100 | 100 |
| 116 | 80.2 | 80.2 | 80.2 | 80.2 | 80.2 | 80.2 |
| 2_116 | 100 | 100 | 100 | 100 | 100 | 100 |
| 1_116 | 99.5 | 98.4 | 100 | 100 | 100 | 100 |
| 237_116 | 99.5 | 98.4 | 100 | 100 | 100 | 100 |
| 4_116 | 99.5 | 98.4 | 100 | 99 | 96.8 | 100 |
| 3_116 | 99.5 | 98.4 | 100 | 100 | 100 | 100 |
| 160 | 80.2 | 80.2 | 80.2 | 80.2 | 80.2 | 80.2 |
| 2_160 | 99.5 | 98.4 | 100 | 100 | 100 | 100 |
| 1_160 | 99.5 | 98.4 | 100 | 100 | 100 | 100 |
| 237_160 | 99.5 | 98.4 | 100 | 98 | 93.5 | 100 |
| 4_160 | 99 | 96.8 | 100 | 99 | 96.8 | 100 |
| 3_160 | 99.5 | 98.4 | 100 | 100 | 100 | 100 |
| 38 | 79.2 | 79.2 | 79.2 | 79.2 | 79.2 | 79.2 |
| 2_38 | 99.5 | 98.4 | 100 | 100 | 100 | 100 |
| 1_38 | 99.5 | 98.4 | 100 | 100 | 100 | 100 |
| 237_38 | 98.5 | 95.2 | 100 | 100 | 100 | 100 |
| 4_38 | 99 | 96.8 | 100 | 99 | 96.8 | 100 |
| 3_38 | 99.5 | 98.4 | 100 | 99 | 96.8 | 100 |
| 99 | 86.2 | 86.2 | 86.2 | 86.2 | 86.2 | 86.2 |
| 2_99 | 99.5 | 98.4 | 100 | 100 | 100 | 100 |
| 1_99 | 99.5 | 98.4 | 100 | 100 | 100 | 100 |
| 237_99 | 99 | 96.7 | 100 | 99 | 96.8 | 100 |
| 4_99 | 99 | 96.7 | 100 | 98 | 93.5 | 100 |
| 3_99 | 99.5 | 98.4 | 100 | 100 | 100 | 100 |
| 104 | 86.3 | 86.3 | 86.3 | 86.3 | 86.3 | 86.3 |
| 2_104 | 99.5 | 98.4 | 100 | 100 | 100 | 100 |
| 1_104 | 100 | 100 | 100 | 100 | 100 | 100 |
| 237_104 | 98.5 | 95.2 | 100 | 98 | 93.5 | 100 |
| 4_104 | 99 | 96.8 | 100 | 99 | 96.8 | 100 |
| 3_104 | 100 | 100 | 100 | 100 | 100 | 100 |

### Comparative Example 1

<Breast Cancer Discriminant Performance of an Existing Tumor Marker in Blood>

The concentrations of the existing tumor marker CEA in blood were measured in the training cohort and the validation cohort obtained in the preceding Reference Examples. When the concentrations of these tumor markers in blood are higher than the reference values described in Non-Patent Literature 3 above (CEA: 5 ng/mL), subjects are usually suspected of having cancer. Thus, whether or not the concentration of CEA in blood exceeded its reference value was confirmed for each sample, and the results were assessed for the ability of the tumor marker to detect cancer in breast cancer patients. The sensitivity of the existing marker in the training cohort and the validation cohort was calculated. The results are shown in Table 5. The sensitivity of CEA was as low as 11.3% in the training cohort and 19.4% in the validation cohort, demonstrating that the marker is not useful in the detection of breast cancer (Table 5).

On the other hand, as shown above in Tables 3 and 6 of Examples 1 and 2, it can be concluded that in all of the polynucleotides consisting of the nucleotide sequences represented by SEQ ID NOs: 1 to 251, combinations of 1 or 2 polynucleotides exhibiting sensitivity beyond the existing breast cancer markers are present, and thus such polynucleotides serve as excellent diagnosis markers.

As shown in these Examples and Comparative Example, the kit, etc., and the method of the present invention can detect breast cancer with higher sensitivity than the existing tumor marker and therefore permit early detection and treatment of breast cancer. As a result, improvement in the survival rate because of the reduced risk of recurrence, and breast conservation therapy as a therapeutic option can also be provided.

### INDUSTRIAL APPLICABILITY

According to the present invention, breast cancer can be effectively detected by a simple and inexpensive method. This enables early detection, diagnosis and treatment of breast cancer. The method of the present invention can detect breast cancer with limited invasiveness using the blood of a patient and therefore allows breast cancer to be detected conveniently and rapidly. All publications, patents, and patent applications cited herein are incorporated herein by reference in their entirety.

1. A method for detecting breast cancer in a human subject, comprising:
measuring expression level of miR-6757-5p in a blood, serum or plasma sample from the subject,
comparing the measured expression level of hsa-miR-6757-5p to a control expression level for a healthy subject;
detecting an increased level of hsa-miR-6757-5p in the sample from the subject as compared to the control expression level from the sample from the healthy subject;
wherein the increased level of hsa-miR-6757-5p indicates that the subject has breast cancer; and
wherein the method further comprises treating the subject for the breast cancer or performing a diagnostic procedure on the subject with the breast cancer:
wherein the treatment comprises surgery, radiotherapy, chemotherapy or a combination thereof; and
wherein the diagnostic procedure comprises mammography, ultrasonography, CT, MRI, abdominal ultrasonography, bone scintigraphy, PET, pathological examination which involves analyzing a lesion tissue under a microscope, or a combination thereof.

2. The method according to claim 1, wherein the expression level of hsa-miR-6757-5p in the sample is measured by using a kit comprising a nucleic acid(s) that specifically binds to hsa-miR-6757-5p.

3. The method according to claim 2, wherein the kit further comprises at least one nucleic acid capable of specifically binding to at least one polynucleotide selected from the group consisting of other breast cancer markers: miR-1307-3p, miR-4634, miR-663a, miR-4532, miR-7704, miR-3178, miR-6729-5p, miR-6090, miR-4732-5p, miR-3184-5p, miR-6727-5p, miR-6088, miR-4674, miR-8073, miR-4787-5p, miR-1469, miR-125a-3p, miR-885-3p, miR-6802-5p, miR-328-5p, miR-6787-5p, miR-8069, miR-6875-5p, miR-1246, miR-4734, miR-6756-5p, miR-3665, miR-6836-3p, miR-6821-5p, miR-6805-5p, miR-4728-5p, miR-6726-5p, miR-197-5p, miR-149-3p, miR-6850-5p, miR-4476, miR-6858-5p, miR-564, miR-4763-3p, miR-575, miR-6771-5p, miR-1231, miR-1908-3p, miR-150-3p, miR-3937, miR-887-3p, miR-3940-5p, miR-4741, miR-6808-5p, miR-6869-5p, miR-5090, miR-615-5p, miR-8072, miR-128-1-5p, miR-1238-5p, miR-365a-5p, miR-204-3p, miR-4492, miR-6785-5p, miR-6511a-5p, miR-4525, miR-1915-5p, miR-3180, miR-6879-5p, miR-1199-5p, miR-6746-5p, miR-711, miR-663b, miR-4707-3p, miR-6893-5p, miR-4675, miR-4638-5p, miR-4651, miR-6087, miR-4665-5p, miR-4758-5p, miR-6887-5p, miR-3620-5p, miR-1909-3p, miR-7641, miR-6724-5p, miR-1343-3p, miR-6780b-5p, miR-4484, miR-4690-5p, miR-4429, miR-1227-5p, miR-4725-3p, miR-6861-5p, miR-6812-5p, miR-3197, miR-8059, miR-3185, miR-4706, miR-4497, miR-3131, miR-6806-5p, miR-187-5p, miR-3180-3p, miR-6848-5p, miR-6820-5p, miR-6800-5p, miR-6717-5p, miR-6795-5p, miR-4632-5p, miR-665, miR-6778-5p, miR-3663-3p, miR-4689, miR-211-3p, miR-6511b-5p, miR-4750-5p, miR-6126, miR-614, miR-7110-5p, miR-744-5p, miR-6769a-5p, miR-4792, miR-5787, miR-6798-5p, miR-6781-5p, miR-4419b, miR-4446-3p, miR-4259, miR-5572, miR-6075, miR-296-3p, miR-6891-5p, miR-4745-5p, miR-6775-5p, miR-6870-5p, miR-920, miR-4530, miR-6819-5p, miR-6825-5p, miR-7847-3p, miR-6131, miR-4433-3p, miR-1228-5p, miR-6743-5p, miR-1268a, miR-3917, miR-6786-5p, miR-3154, miR-638, miR-6741-5p, miR-6889-5p, miR-6840-3p, miR-6510-5p, miR-3188, miR-551b-5p, miR-5001-5p, miR-1268b, miR-7107-5p, miR-6824-5p, miR-6732-5p, miR-371a-5p, miR-6794-5p, miR-6779-5p, miR-4271, miR-5195-3p, miR-6762-5p, miR-939-5p, miR-1247-3p, miR-6777-5p, miR-6722-3p, miR-3656, miR-4688, miR-3195, miR-6766-5p, miR-4447, miR-4656, miR-7108-5p, miR-3191-3p, miR-1273g-3p, miR-4463, miR-2861, miR-3196, miR-6877-5p, miR-3679-5p, miR-4442, miR-6789-5p, miR-6782-5p, miR-486-3p, miR-6085, miR-4746-3p, miR-619-5p, miR-937-5p, miR-6803-5p, miR-4298, miR-4454, miR-4459, miR-7150, miR-6880-5p, miR-4449, miR-8063, miR-4695-5p, miR-6132, miR-6829-5p, miR-4486, miR-6805-3p, miR-6826-5p, miR-4508, miR-1343-5p, miR-7114-5p, miR-3622a-5p, miR-6765-5p, miR-7845-5p, miR-3960, miR-6749-5p, miR-1260b, miR-6799-5p, miR-4723-5p, miR-6784-5p, miR-5100, miR-6769b-5p, miR-1207-5p, miR-642a-3p, miR-4505, miR-4270, miR-6721-5p, miR-7111-5p, miR-6791-5p, miR-7109-5p, miR-4258, miR-6515-3p, miR-6851-5p, miR-6125, miR-4749-5p, miR-4726-5p, miR-4513, miR-6089, miR-6816-5p, miR-4466, miR-4488, miR-6752-5p and miR-4739, and/or miR-760, miR-602, miR-423-5p, miR-92a-2-5p, miR-16-5p, miR-451a, miR-135a-3p, miR-486-5p, miR-4257, miR-92b-5p, miR-1915-3p, miR-718, miR-940, miR-296-5p, miR-23b-3p, miR-92a-3p, miR-658, miR-6842-5p, miR-6124, miR-6765-3p, miR-7106-5p, miR-4534, miR-92b-3p, miR-3135b, miR-4687-3p, miR-762, miR-3619-3p, miR-4467, miR-557, miR-1237-5p, miR-1908-5p, miR-4286, miR-6885-5p and miR-6763-5p.

4. The method according to claim 1, comprising performing the diagnostic procedure on the subject.

5. The method according to claim 1, wherein the expression level of hsa-miR-6757-5p in the sample is measured by using a device comprising a nucleic acid(s) that specifically binds to hsa-miR-6757-5p.

6. The method according to claim 5, wherein the device further comprises at least one nucleic acid capable of specifically binding to at least one polynucleotide selected from the group consisting of other breast cancer markers: miR-1307-3p, miR-4634, miR-663a, miR-4532, miR-7704, miR-3178, miR-6729-5p, miR-6090, miR-4732-5p, miR-3184-5p, miR-6727-5p, miR-6088, miR-4674, miR-8073, miR-4787-5p, miR-1469, miR-125a-3p, miR-885-3p, miR-6802-5p, miR-328-5p, miR-6787-5p, miR-8069, miR-6875-5p, miR-1246, miR-4734, miR-6756-5p, miR-3665, miR-6836-3p, miR-6821-5p, miR-6805-5p, miR-4728-5p, miR-6726-5p, miR-197-5p, miR-149-3p, miR-6850-5p, miR-4476, miR-6858-5p, miR-564, miR-4763-3p, miR-575, miR-6771-5p, miR-1231, miR-1908-3p, miR-150-3p, miR-3937, miR-887-3p, miR-3940-5p, miR-4741, miR-6808-5p, miR-6869-5p, miR-5090, miR-615-5p, miR-8072, miR-128-1-5p, miR-1238-5p, miR-365a-5p, miR-204-3p, miR-4492, miR-6785-5p, miR-6511a-5p, miR-4525, miR-1915-5p, miR-3180, miR-6879-5p, miR-1199-5p, miR-6746-5p, miR-711, miR-663b, miR-4707-3p, miR-6893-5p, miR-4675, miR-4638-5p, miR-4651, miR-6087, miR-4665-5p, miR-4758-5p, miR-6887-5p, miR-3620-5p, miR-1909-3p, miR-7641, miR-6724-5p, miR-1343-3p, miR-6780b-5p, miR-4484, miR-4690-5p, miR-4429, miR-1227-5p, miR-4725-3p, miR-6861-5p, miR-6812-5p, miR-3197, miR-8059, miR-3185, miR-4706, miR-4497, miR-3131, miR-6806-5p, miR-187-5p, miR-3180-3p, miR-6848-5p, miR-6820-5p, miR-6800-5p, miR-6717-5p, miR-6795-5p, miR-4632-5p, miR-665, miR-6778-5p, miR-3663-3p, miR-4689, miR-211-3p, miR-6511b-5p, miR-4750-5p, miR-6126, miR-614, miR-7110-5p, miR-744-5p, miR-6769a-5p, miR-4792, miR-5787, miR-6798-5p, miR-6781-5p, miR-4419b, miR-4446-3p, miR-4259, miR-5572, miR-6075, miR-296-3p, miR-6891-5p, miR-4745-5p, miR-6775-5p, miR-6870-5p, miR-920, miR-4530, miR-6819-5p, miR-6825-5p, miR-7847-3p, miR-6131, miR-4433-3p, miR-1228-5p, miR-6743-5p, miR-1268a, miR-3917, miR-6786-5p, miR-3154, miR-638, miR-6741-5p, miR-6889-5p, miR-6840-3p, miR-6510-5p, miR-3188, miR-551b-5p, miR-5001-5p, miR-1268b, miR-7107-5p, miR-6824-5p, miR-6732-5p, miR-371a-5p, miR-6794-5p, miR-6779-5p, miR-4271, miR-5195-3p, miR-6762-5p, miR-939-5p, miR-1247-3p, miR-6777-5p, miR-6722-3p, miR-3656, miR-4688, miR-3195, miR-6766-5p, miR-4447, miR-4656, miR-7108-5p, miR-3191-3p, miR-1273g-3p, miR-4463, miR-2861, miR-3196, miR-6877-5p, miR-3679-5p, miR-4442, miR-6789-5p, miR-6782-5p, miR-486-3p, miR-6085, miR-4746-3p, miR-619-5p, miR-937-5p, miR-6803-5p, miR-4298, miR-4454, miR-4459, miR-7150, miR-6880-5p, miR-4449, miR-8063, miR-4695-5p, miR-6132, miR-6829-5p, miR-4486, miR-6805-3p, miR-6826-5p, miR-4508, miR-1343-5p, miR-7114-5p, miR-3622a-5p, miR-6765-5p, miR-7845-5p, miR-3960, miR-6749-5p, miR-1260b, miR-6799-5p, miR-4723-5p, miR-6784-5p, miR-5100, miR-6769b-5p, miR-1207-5p, miR-642a-3p, miR-4505, miR-4270, miR-6721-5p, miR-7111-5p, miR-6791-5p, miR-7109-5p, miR-4258, miR-6515-3p, miR-6851-5p, miR-6125, miR-4749-5p, miR-4726-5p, miR-4513, miR-6089, miR-6816-5p, miR-4466, miR-4488, miR-6752-5p and miR-4739, and/or miR-760, miR-602, miR-423-5p, miR-92a-2-5p, miR-16-5p, miR-451a, miR-135a-3p, miR-486-5p, miR-4257, miR-92b-5p, miR-1915-3p, miR-718, miR-940, miR-296-5p, miR-23b-3p, miR-92a-3p, miR-658, miR-6842-5p, miR-6124, miR-6765-3p, miR-7106-5p, miR-4534, miR-92b-3p, miR-3135b, miR-4687-3p, miR-762, miR-3619-3p, miR-4467, miR-557, miR-1237-5p, miR-1908-5p, miR-4286, miR-6885-5p and miR-6763-5p.


SEQUENCE LISTING (871 sequences)

SEQ ID NO: 1 - RNA, 23 nt, Homo sapiens
  source 1..23: mol_type = unassigned RNA
ccccggtgttggggcgcgtctgc

SEQ ID NO: 2 - RNA, 23 nt, Homo sapiens
  source 1..23: mol_type = unassigned RNA
ctggcggagcccattccatgcca

SEQ ID NO: 3 - RNA, 22 nt, Homo sapiens
  source 1..22: mol_type = unassigned RNA
actcggcgtggcgtcggtcgtg

SEQ ID NO: 4 - RNA, 19 nt, Homo sapiens
  source 1..19: mol_type = unassigned RNA
cggcgcgaccggcccgggg

SEQ ID NO: 5 - RNA, 22 nt, Homo sapiens
  source 1..22: mol_type = unassigned RNA
aggcggggcgccgcgggaccgc

SEQ ID NO: 6 - RNA, 17 nt, Homo sapiens
  source 1..17: mol_type = unassigned RNA
ccccggggagcccggcg

SEQ ID NO: 7 - RNA, 19 nt, Homo sapiens
  source 1..19: mol_type = unassigned RNA
cggggtcggcggcgacgtg

SEQ ID NO: 8 - RNA, 17 nt, Homo sapiens
  source 1..17: mol_type = unassigned RNA
ggggcgcggccggatcg

SEQ ID NO: 9 - RNA, 22 nt, Homo sapiens
  source 1..22: mol_type = unassigned RNA
tgggcgagggcggctgagcggc

SEQ ID NO: 10 - RNA, 19 nt, Homo sapiens
  source 1..19: mol_type = unassigned RNA
ggggagcgaggggcggggc

SEQ ID NO: 11 - RNA, 23 nt, Homo sapiens
  source 1..23: mol_type = unassigned RNA
tgtagagcagggagcaggaagct

SEQ ID NO: 12 - RNA, 24 nt, Homo sapiens
  source 1..24: mol_type = unassigned RNA
tgaggggcctcagaccgagctttt

SEQ ID NO: 13 - RNA, 23 nt, Homo sapiens
  source 1..23: mol_type = unassigned RNA
ctcggggcaggcggctgggagcg

SEQ ID NO: 14 - RNA, 20 nt, Homo sapiens
  source 1..20: mol_type = unassigned RNA
agagatgaagcgggggggcg

SEQ ID NO: 15 - RNA, 21 nt, Homo sapiens
  source 1..21: mol_type = unassigned RNA
ctgggctcgggacgcgcggct

SEQ ID NO: 16 - RNA, 22 nt, Homo sapiens
  source 1..22: mol_type = unassigned RNA
acctggcagcagggagcgtcgt

SEQ ID NO: 17 - RNA, 22 nt, Homo sapiens
  source 1..22: mol_type = unassigned RNA
gcgggggtggcggcggcatccc

SEQ ID NO: 18 - RNA, 22 nt, Homo sapiens
  source 1..22: mol_type = unassigned RNA
ctcggcgcggggcgcgggctcc

SEQ ID NO: 19 - RNA, 22 nt, Homo sapiens
  source 1..22: mol_type = unassigned RNA
acaggtgaggttcttgggagcc

SEQ ID NO: 20 - RNA, 22 nt, Homo sapiens
  source 1..22: mol_type = unassigned RNA
agtgggaggccagggcacggca

SEQ ID NO: 21 - RNA, 22 nt, Homo sapiens
  source 1..22: mol_type = unassigned RNA
aggcagcggggtgtagtggata

SEQ ID NO: 22 - RNA, 20 nt, Homo sapiens
  source 1..20: mol_type = unassigned RNA
ctaggtggggggcttgaagc

SEQ ID NO: 23 - RNA, 23 nt, Homo sapiens
  source 1..23: mol_type = unassigned RNA
gggggggcaggaggggctcaggg

SEQ ID NO: 24 - RNA, 22 nt, Homo sapiens
  source 1..22: mol_type = unassigned RNA
tggcgggggtagagctggctgc

SEQ ID NO: 25 - RNA, 23 nt, Homo sapiens
  source 1..23: mol_type = unassigned RNA
ggatggttgggggcggtcggcgt

SEQ ID NO: 26 - RNA, 21 nt, Homo sapiens
  source 1..21: mol_type = unassigned RNA
tgagggacccaggacaggaga

SEQ ID NO: 27 - RNA, 19 nt, Homo sapiens
  source 1..19: mol_type = unassigned RNA
aatggatttttggagcagg

SEQ ID NO: 28 - RNA, 22 nt, Homo sapiens
  source 1..22: mol_type = unassigned RNA
gctgcgggctgcggtcagggcg

SEQ ID NO: 29 - RNA, 22 nt, Homo sapiens
  source 1..22: mol_type = unassigned RNA
tagggatgggaggccaggatga

SEQ ID NO: 30 - RNA, 23 nt, Homo sapiens
  source 1..23: mol_type = unassigned RNA
agggtggggctggaggtggggct

SEQ ID NO: 31 - RNA, 18 nt, Homo sapiens
  source 1..18: mol_type = unassigned RNA
agcaggtgcggggcggcg

SEQ ID NO: 32 - RNA, 21 nt, Homo sapiens
  source 1..21: mol_type = unassigned RNA
atgcctcccccggccccgcag

SEQ ID NO: 33 - RNA, 23 nt, Homo sapiens
  source 1..23: mol_type = unassigned RNA
gtgcgtggtggctcgaggcgggg

SEQ ID NO: 34 - RNA, 22 nt, Homo sapiens
  source 1..22: mol_type = unassigned RNA
tagggggcggcttgtggagtgt

SEQ ID NO: 35 - RNA, 23 nt, Homo sapiens
  source 1..23: mol_type = unassigned RNA
tgggaggggagaggcagcaagca

SEQ ID NO: 36 - RNA, 21 nt, Homo sapiens
  source 1..21: mol_type = unassigned RNA
cgggagctggggtctgcaggt

SEQ ID NO: 37 - RNA, 23 nt, Homo sapiens
  source 1..23: mol_type = unassigned RNA
cgggtagagagggcagtgggagg

SEQ ID NO: 38 - RNA, 21 nt, Homo sapiens
  source 1..21: mol_type = unassigned RNA
agggagggacgggggctgtgc

SEQ ID NO: 39 - RNA, 22 nt, Homo sapiens
  source 1..22: mol_type = unassigned RNA
gtgcggaacgctggccggggcg

SEQ ID NO: 40 - RNA, 22 nt, Homo sapiens
  source 1..22: mol_type = unassigned RNA
caggaaggatttagggacaggc

SEQ ID NO: 41 - RNA, 22 nt, Homo sapiens
  source 1..22: mol_type = unassigned RNA
gtgaggaggggctggcagggac

SEQ ID NO: 42 - RNA, 19 nt, Homo sapiens
  source 1..19: mol_type = unassigned RNA
aggcacggtgtcagcaggc

SEQ ID NO: 43 - RNA, 24 nt, Homo sapiens
  source 1..24: mol_type = unassigned RNA
aggcaggggctggtgctgggcggg

SEQ ID NO: 44 - RNA, 19 nt, Homo sapiens
  source 1..19: mol_type = unassigned RNA
gagccagttggacaggagc

SEQ ID NO: 45 - RNA, 22 nt, Homo sapiens
  source 1..22: mol_type = unassigned RNA
ctcgggagggcatgggccaggc

SEQ ID NO: 46 - RNA, 20 nt, Homo sapiens
  source 1..20: mol_type = unassigned RNA
gtgtctgggcggacagctgc

SEQ ID NO: 47 - RNA, 21 nt, Homo sapiens
  source 1..21: mol_type = unassigned RNA
ccggccgccggctccgccccg

SEQ ID NO: 48 - RNA, 22 nt, Homo sapiens
  source 1..22: mol_type = unassigned RNA
ctggtacaggcctgggggacag

SEQ ID NO: 49 - RNA, 23 nt, Homo sapiens
  source 1..23: mol_type = unassigned RNA
acaggcggctgtagcaatggggg

SEQ ID NO: 50 - RNA, 22 nt, Homo sapiens
  source 1..22: mol_type = unassigned RNA
gtgaacgggcgccatcccgagg

SEQ ID NO: 51 - RNA, 20 nt, Homo sapiens
  source 1..20: mol_type = unassigned RNA
gtgggttggggcgggctctg

SEQ ID NO: 52 - RNA, 23 nt, Homo sapiens
  source 1..23: mol_type = unassigned RNA
cgggctgtccggaggggtcggct

SEQ ID NO: 53 - RNA, 22 nt, Homo sapiens
  source 1..22: mol_type = unassigned RNA
caggcagggaggtgggaccatg

SEQ ID NO: 54 - RNA, 22 nt, Homo sapiens
  source 1..22: mol_type = unassigned RNA
gtgagtagtggcgcgcggcggc

SEQ ID NO: 55 - RNA, 23 nt, Homo sapiens
  source 1..23: mol_type = unassigned RNA
ccggggcagattggtgtagggtg

SEQ ID NO: 56 - RNA, 22 nt, Homo sapiens
  source 1..22: mol_type = unassigned RNA
gggggtccccggtgctcggatc

SEQ ID NO: 57 - RNA, 20 nt, Homo sapiens
  source 1..20: mol_type = unassigned RNA
ggcggcggggaggtaggcag

SEQ ID NO: 58 - RNA, 23 nt, Homo sapiens
  source 1..23: mol_type = unassigned RNA
cggggccgtagcactgtctgaga

SEQ ID NO: 59 - RNA, 23 nt, Homo sapiens
  source 1..23: mol_type = unassigned RNA
gtgagtgggagccccagtgtgtg

SEQ ID NO: 60 - RNA, 23 nt, Homo sapiens
  source 1..23: mol_type = unassigned RNA
agggacttttgggggcagatgtg

SEQ ID NO: 61 - RNA, 21 nt, Homo sapiens
  source 1..21: mol_type = unassigned RNA
gctgggaaggcaaagggacgt

SEQ ID NO: 62 - RNA, 17 nt, Homo sapiens
  source 1..17: mol_type = unassigned RNA
ggggctgggcgcgcgcc

SEQ ID NO: 63 - RNA, 22 nt, Homo sapiens
  source 1..22: mol_type = unassigned RNA
tgggagggcgtggatgatggtg

SEQ ID NO: 64 - RNA, 23 nt, Homo sapiens
  source 1..23: mol_type = unassigned RNA
caggcagaagtggggctgacagg

SEQ ID NO: 65 - RNA, 21 nt, Homo sapiens
  source 1..21: mol_type = unassigned RNA
ggggggatgtgcatgctggtt

SEQ ID NO: 66 - RNA, 22 nt, Homo sapiens
  source 1..22: mol_type = unassigned RNA
accttgccttgctgcccgggcc

SEQ ID NO: 67 - RNA, 19 nt, Homo sapiens
  source 1..19: mol_type = unassigned RNA
tggggcggagcttccggag

SEQ ID NO: 68 - RNA, 22 nt, Homo sapiens
  source 1..22: mol_type = unassigned RNA
cagggcagggaaggtgggagag

SEQ ID NO: 69 - RNA, 20 nt, Homo sapiens
  source 1..20: mol_type = unassigned RNA
cctgagcccgggccgcgcag

SEQ ID NO: 70 - RNA, 22 nt, Homo sapiens
  source 1..22: mol_type = unassigned RNA
ccgggagaaggaggtggcctgg

SEQ ID NO: 71 - RNA, 22 nt, Homo sapiens
  source 1..22: mol_type = unassigned RNA
gggacccagggagagacgtaag

SEQ ID NO: 72 - RNA, 22 nt, Homo sapiens
  source 1..22: mol_type = unassigned RNA
ggtggcccggccgtgcctgagg

SEQ ID NO: 73 - RNA, 22 nt, Homo sapiens
  source 1..22: mol_type = unassigned RNA
agcccgccccagccgaggttct

SEQ ID NO: 74 - RNA, 21 nt, Homo sapiens
  source 1..21: mol_type = unassigned RNA
caggcaggtgtagggtggagc

SEQ ID NO: 75 - RNA, 22 nt, Homo sapiens
  source 1..22: mol_type = unassigned RNA
ggggctgtgattgaccagcagg

SEQ ID NO: 76 - RNA, 21 nt, Homo sapiens
  source 1..21: mol_type = unassigned RNA
actcggctgcggtggacaagt

SEQ ID NO: 77 - RNA, 20 nt, Homo sapiens
  source 1..20: mol_type = unassigned RNA
cggggtgggtgaggtcgggc

SEQ ID NO: 78 - RNA, 18 nt, Homo sapiens
  source 1..18: mol_type = unassigned RNA
tgaggcgggggggcgagc

SEQ ID NO: 79 - RNA, 23 nt, Homo sapiens
  source 1..23: mol_type = unassigned RNA
ctgggggacgcgtgagcgcgagc

SEQ ID NO: 80 - RNA, 23 nt, Homo sapiens
  source 1..23: mol_type = unassigned RNA
gtgagtgggagccggtggggctg

SEQ ID NO: 81 - RNA, 23 nt, Homo sapiens
  source 1..23: mol_type = unassigned RNA
tggggggacagatggagaggaca

SEQ ID NO: 82 - RNA, 22 nt, Homo sapiens
  source 1..22: mol_type = unassigned RNA
gtgggctgggctgggctgggcc

SEQ ID NO: 83 - RNA, 22 nt, Homo sapiens
  source 1..22: mol_type = unassigned RNA
cgcaggggccgggtgctcaccg

SEQ ID NO: 84 - RNA, 19 nt, Homo sapiens
  source 1..19: mol_type = unassigned RNA
ttgatctcggaagctaagc

SEQ ID NO: 85 - RNA, 23 nt, Homo sapiens
  source 1..23: mol_type = unassigned RNA
ctgggcccgcggcgggcgtgggg

SEQ ID NO: 86 - RNA, 22 nt, Homo sapiens
  source 1..22: mol_type = unassigned RNA
ctcctggggcccgcactctcgc

SEQ ID NO: 87 - RNA, 23 nt, Homo sapiens
  source 1..23: mol_type = unassigned RNA
tggggaaggcttggcagggaaga

SEQ ID NO: 88 - RNA, 20 nt, Homo sapiens
  source 1..20: mol_type = unassigned RNA
aaaaggcgggagaagcccca

SEQ ID NO: 89 - RNA, 22 nt, Homo sapiens
  source 1..22: mol_type = unassigned RNA
gagcaggcgaggctgggctgaa

SEQ ID NO: 90 - RNA, 20 nt, Homo sapiens
  source 1..20: mol_type = unassigned RNA
aaaagctgggctgagaggcg

SEQ ID NO: 91 - RNA, 17 nt, Homo sapiens
  source 1..17: mol_type = unassigned RNA
gtggggccaggcggtgg

SEQ ID NO: 92 - RNA, 22 nt, Homo sapiens
  source 1..22: mol_type = unassigned RNA
tggggaaggcgtcagtgtcggg

SEQ ID NO: 93 - RNA, 22 nt, Homo sapiens
  source 1..22: mol_type = unassigned RNA
actgggtaggtggggctccagg

SEQ ID NO: 94 - RNA, 25 nt, Homo sapiens
  source 1..25: mol_type = unassigned RNA
atggggtgagatggggaggagcagc

SEQ ID NO: 95 - RNA, 23 nt, Homo sapiens
  source 1..23: mol_type = unassigned RNA
ggaggcgcaggctcggaaaggcg

SEQ ID NO: 96 - RNA, 22 nt, Homo sapiens
  source 1..22: mol_type = unassigned RNA
ggggaactgtagatgaaaaggc

SEQ ID NO: 97 - RNA, 23 nt, Homo sapiens
  source 1..23: mol_type = unassigned RNA
agaagaaggcggtcggtctgcgg

SEQ ID NO: 98 - RNA, 25 nt, Homo sapiens
  source 1..25: mol_type = unassigned RNA
agcggggaggaagtgggcgctgctt

SEQ ID NO: 99 - RNA, 17 nt, Homo sapiens
  source 1..17: mol_type = unassigned RNA
ctccgggacggctgggc

SEQ ID NO: 100 - RNA, 23 nt, Homo sapiens
  source 1..23: mol_type = unassigned RNA
tcgaggactggtggaagggcctt

SEQ ID NO: 101 - RNA, 24 nt, Homo sapiens
  source 1..24: mol_type = unassigned RNA
tgtaggcatgaggcagggcccagg

SEQ ID NO: 102 - RNA, 22 nt, Homo sapiens
  source 1..22: mol_type = unassigned RNA
ggctacaacacaggacccgggc

SEQ ID NO: 103 - RNA, 22 nt, Homo sapiens
  source 1..22: mol_type = unassigned RNA
tggggcggagcttccggaggcc

SEQ ID NO: 104 - RNA, 23 nt, Homo sapiens
  source 1..23: mol_type = unassigned RNA
tgggggctgggatgggccatggt

SEQ ID NO: 105 - RNA, 19 nt, Homo sapiens
  source 1..19: mol_type = unassigned RNA
tgcggcagagctggggtca

SEQ ID NO: 106 - RNA, 21 nt, Homo sapiens
  source 1..21: mol_type = unassigned RNA
gtaggtgacagtcaggggcgg

SEQ ID NO: 107 - RNA, 22 nt, Homo sapiens
  source 1..22: mol_type = unassigned RNA
aggcgatgtggggatgtagaga

SEQ ID NO: 108 - RNA, 24 nt, Homo sapiens
  source 1..24: mol_type = unassigned RNA
tggggggacaggatgagaggctgt

SEQ ID NO: 109 - RNA, 23 nt, Homo sapiens
  source 1..23: mol_type = unassigned RNA
gagggcagcgtgggtgtggcgga

SEQ ID NO: 110 - RNA, 20 nt, Homo sapiens
  source 1..20: mol_type = unassigned RNA
accaggaggctgaggcccct

SEQ ID NO: 111 - RNA, 22 nt, Homo sapiens
  source 1..22: mol_type = unassigned RNA
agtgggaggacaggaggcaggt

SEQ ID NO: 112 - RNA, 23 nt, Homo sapiens
  source 1..23: mol_type = unassigned RNA
tgagcaccacacaggccgggcgc

SEQ ID NO: 113 - RNA, 22 nt, Homo sapiens
  source 1..22: mol_type = unassigned RNA
ttgaggagacatggtgggggcc

SEQ ID NO: 114 - RNA, 21 nt, Homo sapiens
  source 1..21: mol_type = unassigned RNA
gcagggacagcaaaggggtgc

SEQ ID NO: 115 - RNA, 24 nt, Homo sapiens
  source 1..24: mol_type = unassigned RNA
ctgcaggcagaagtggggctgaca

SEQ ID NO: 116 - RNA, 22 nt, Homo sapiens
  source 1..22: mol_type = unassigned RNA
ctcgggcggaggtggttgagtg

SEQ ID NO: 117 - RNA, 18 nt, Homo sapiens
  source 1..18: mol_type = unassigned RNA
gtgaaggcccggcggaga

SEQ ID NO: 118 - RNA, 23 nt, Homo sapiens
  source 1..23: mol_type = unassigned RNA
gaacgcctgttcttgccaggtgg

SEQ ID NO: 119 - RNA, 21 nt, Homo sapiens
  source 1..21: mol_type = unassigned RNA
tgggggtgtggggagagagag

SEQ ID NO: 120 - RNA, 22 nt, Homo sapiens
  source 1..22: mol_type = unassigned RNA
tgcggggctagggctaacagca

SEQ ID NO: 121 - RNA, 21 nt, Homo sapiens
  source 1..21: mol_type = unassigned RNA
aggtgggtatggaggagccct

SEQ ID NO: 122 - RNA, 18 nt, Homo sapiens
  source 1..18: mol_type = unassigned RNA
cggtgagcgctcgctggc

SEQ ID NO: 123 - RNA, 20 nt, Homo sapiens
  source 1..20: mol_type = unassigned RNA
gggctggggcgcggggaggt

SEQ ID NO: 124 - RNA, 23 nt, Homo sapiens
  source 1..23: mol_type = unassigned RNA
ccagggggatgggcgagcttggg

SEQ ID NO: 125 - RNA, 21 nt, Homo sapiens
  source 1..21: mol_type = unassigned RNA
cgggccggaggtcaagggcgt

SEQ ID NO: 126 - RNA, 18 nt, Homo sapiens
  source 1..18: mol_type = unassigned RNA
gaggctgaaggaagatgg

SEQ ID NO: 127 - RNA, 22 nt, Homo sapiens
  source 1..22: mol_type = unassigned RNA
cagggctggcagtgacatgggt

SEQ ID NO: 128 - RNA, 22 nt, Homo sapiens
  source 1..22: mol_type = unassigned RNA
cagttgggtctaggggtcagga

SEQ ID NO: 129 - RNA, 21 nt, Homo sapiens
  source 1..21: mol_type = unassigned RNA
gttggggtgcaggggtctgct

SEQ ID NO: 130 - RNA, 21 nt, Homo sapiens
  source 1..21: mol_type = unassigned RNA
acggcccaggcggcattggtg

SEQ ID NO: 131 - RNA, 22 nt, Homo sapiens
  source 1..22: mol_type = unassigned RNA
gagggttgggtggaggctctcc

SEQ ID NO: 132 - RNA, 19 nt, Homo sapiens
  source 1..19: mol_type = unassigned RNA
taaggagggggatgagggg

SEQ ID NO: 133 - RNA, 23 nt, Homo sapiens
  source 1..23: mol_type = unassigned RNA
tgagtggggctcccgggacggcg

SEQ ID NO: 134 - RNA, 25 nt, Homo sapiens
  source 1..25: mol_type = unassigned RNA
tcggggcatgggggagggaggctgg

SEQ ID NO: 135 - RNA, 19 nt, Homo sapiens
  source 1..19: mol_type = unassigned RNA
tgggggagatgggggttga

SEQ ID NO: 136 - RNA, 20 nt, Homo sapiens
  source 1..20: mol_type = unassigned RNA
ggggagctgtggaagcagta

SEQ ID NO: 137 - RNA, 18 nt, Homo sapiens
  source 1..18: mol_type = unassigned RNA
cccagcaggacgggagcg

SEQ ID NO: 138 - RNA, 22 nt, Homo sapiens
  source 1..22: mol_type = unassigned RNA
ttggggtggagggccaaggagc

SEQ ID NO: 139 - RNA, 22 nt, Homo sapiens
  source 1..22: mol_type = unassigned RNA
tggggaggtgtggagtcagcat

SEQ ID NO: 140 - RNA, 21 nt, Homo sapiens
  source 1..21: mol_type = unassigned RNA
cgtggaggacgaggaggaggc

SEQ ID NO: 141 - RNA, 19 nt, Homo sapiens
  source 1..19: mol_type = unassigned RNA
ggctggtcagatgggagtg

SEQ ID NO: 142 - RNA, 21 nt, Homo sapiens
  source 1..21: mol_type = unassigned RNA
acaggagtgggggtgggacat

SEQ ID NO: 143 - RNA, 21 nt, Homo sapiens
  source 1..21: mol_type = unassigned RNA
gtgggcgggggcaggtgtgtg

SEQ ID NO: 144 - RNA, 22 nt, Homo sapiens
  source 1..22: mol_type = unassigned RNA
aaggggcagggacgggtggccc

SEQ ID NO: 145 - RNA, 18 nt, Homo sapiens
  source 1..18: mol_type = unassigned RNA
cgggcgtggtggtggggg

SEQ ID NO: 146 - RNA, 20 nt, Homo sapiens
  source 1..20: mol_type = unassigned RNA
gctcggactgagcaggtggg

SEQ ID NO: 147 - RNA, 21 nt, Homo sapiens
  source 1..21: mol_type = unassigned RNA
gcggtggggccggaggggcgt

SEQ ID NO: 148 - RNA, 22 nt, Homo sapiens
  source 1..22: mol_type = unassigned RNA
cagaaggggagttgggagcaga

SEQ ID NO: 149 - RNA, 25 nt, Homo sapiens
  source 1..25: mol_type = unassigned RNA
agggatcgcgggcgggtggcggcct

SEQ ID NO: 150 - RNA, 22 nt, Homo sapiens
  source 1..22: mol_type = unassigned RNA
gtgggtgctggtgggagccgtg

SEQ ID NO: 151 - RNA, 23 nt, Homo sapiens
  source 1..23: mol_type = unassigned RNA
tcggggagtctggggtccggaat

SEQ ID NO: 152 - RNA, 22 nt, Homo sapiens
  source 1..22: mol_type = unassigned RNA
gcccaggactttgtgcggggtg

SEQ ID NO: 153 - RNA, 22 nt, Homo sapiens
  source 1..22: mol_type = unassigned RNA
cagcaggggagagagaggagtc

SEQ ID NO: 154 - RNA, 23 nt, Homo sapiens
  source 1..23: mol_type = unassigned RNA
agaggctttgtgcggatacgggg

SEQ ID NO: 155 - RNA, 22 nt, Homo sapiens
  source 1..22: mol_type = unassigned RNA
gaaatcaagcgtgggtgagacc

SEQ ID NO: 156 - RNA, 24 nt, Homo sapiens
  source 1..24: mol_type = unassigned RNA
agggctggactcagcggcggagct

SEQ ID NO: 157 - RNA, 20 nt, Homo sapiens
  source 1..20: mol_type = unassigned RNA
cgggcgtggtggtgggggtg

SEQ ID NO: 158 - RNA, 22 nt, Homo sapiens
  source 1..22: mol_type = unassigned RNA
tcggcctggggaggaggaaggg

SEQ ID NO: 159 - RNA, 22 nt, Homo sapiens
  source 1..22: mol_type = unassigned RNA
gtaggggaggttgggccaggga

SEQ ID NO: 160 - RNA, 20 nt, Homo sapiens
  source 1..20: mol_type = unassigned RNA
tagggggtggcaggctggcc

SEQ ID NO: 161 - RNA, 20 nt, Homo sapiens
  source 1..20: mol_type = unassigned RNA
actcaaactgtgggggcact

SEQ ID NO: 162 - RNA, 20 nt, Homo sapiens
  source 1..20: mol_type = unassigned RNA
cagggggactgggggtgagc

SEQ ID NO: 163 - RNA, 21 nt, Homo sapiens
  source 1..21: mol_type = unassigned RNA
ctgggaggggctgggtttggc

SEQ ID NO: 164 - RNA, 19 nt, Homo sapiens
  source 1..19: mol_type = unassigned RNA
gggggaagaaaaggtgggg

SEQ ID NO: 165 - RNA, 21 nt, Homo sapiens
  source 1..21: mol_type = unassigned RNA
atccagttctctgagggggct

SEQ ID NO: 166 - RNA, 23 nt, Homo sapiens
  source 1..23: mol_type = unassigned RNA
cggggccatggagcagcctgtgt

SEQ ID NO: 167 - RNA, 24 nt, Homo sapiens
  source 1..24: mol_type = unassigned RNA
tggggagctgaggctctgggggtg

SEQ ID NO: 168 - RNA, 24 nt, Homo sapiens
  source 1..24: mol_type = unassigned RNA
ccccgggaacgtcgagactggagc

SEQ ID NO: 169 - RNA, 23 nt, Homo sapiens
  source 1..23: mol_type = unassigned RNA
acggggagtcaggcagtggtgga

SEQ ID NO: 170 - RNA, 22 nt, Homo sapiens
  source 1..22: mol_type = unassigned RNA
tgcaggggtcgggtgggccagg

SEQ ID NO: 171 - RNA, 17 nt, Homo sapiens
  source 1..17: mol_type = unassigned RNA
ggcgggtgcgggggtgg

SEQ ID NO: 172 - RNA, 22 nt, Homo sapiens
  source 1..22: mol_type = unassigned RNA
taggggcagcagaggacctggg

SEQ ID NO: 173 - RNA, 17 nt, Homo sapiens
  source 1..17: mol_type = unassigned RNA
cgcgccgggcccgggtt

SEQ ID NO: 174 - RNA, 24 nt, Homo sapiens
  source 1..24: mol_type = unassigned RNA
cgggtgggagcagatcttattgag

SEQ ID NO: 175 - RNA, 17 nt, Homo sapiens
  source 1..17: mol_type = unassigned RNA
ggtgggggctgttgttt

SEQ ID NO: 176 - RNA, 23 nt, Homo sapiens
  source 1..23: mol_type = unassigned RNA
tgggctgagggcaggaggcctgt

SEQ ID NO: 177 - RNA, 21 nt, Homo sapiens
  source 1..21: mol_type = unassigned RNA
gtgtggccggcaggcgggtgg

SEQ ID NO: 178 - RNA, 23 nt, Homo sapiens
  source 1..23: mol_type = unassigned RNA
tggggacgtagctggccagacag

SEQ ID NO: 179 - RNA, 21 nt, Homo sapiens
  source 1..21: mol_type = unassigned RNA
accactgcactccagcctgag

SEQ ID NO: 180 - RNA, 17 nt, Homo sapiens
  source 1..17: mol_type = unassigned RNA
gagactggggtggggcc

SEQ ID NO: 181 - RNA, 19 nt, Homo sapiens
  source 1..19: mol_type = unassigned RNA
ggggcctggcggtgggcgg

SEQ ID NO: 182 - RNA, 18 nt, Homo sapiens
  source 1..18: mol_type = unassigned RNA
cggggcggcaggggcctc

SEQ ID NO: 183 - RNA, 22 nt, Homo sapiens
  source 1..22: mol_type = unassigned RNA
agggccgaagggtggaagctgc

SEQ ID NO: 184 - RNA, 23 nt, Homo sapiens
  source 1..23: mol_type = unassigned RNA
tgaggatatggcagggaagggga

SEQ ID NO: 185 - RNA, 17 nt, Homo sapiens
  source 1..17: mol_type = unassigned RNA
gccggacaagagggagg

SEQ ID NO: 186 - RNA, 24 nt, Homo sapiens
  source 1..24: mol_type = unassigned RNA
gtaggggcgtcccgggcgcgcggg

SEQ ID NO: 187 - RNA, 25 nt, Homo sapiens
  source 1..25: mol_type = unassigned RNA
taggggtgggggaattcaggggtgt

SEQ ID NO: 188 - RNA, 21 nt, Homo sapiens
  source 1..21: mol_type = unassigned RNA
cggggcagctcagtacaggat

SEQ ID NO: 189 - RNA, 19 nt, Homo sapiens
  source 1..19: mol_type = unassigned RNA
aaggggctgggggagcaca

SEQ ID NO: 190 - RNA, 21 nt, Homo sapiens
  source 1..21: mol_type = unassigned RNA
agcggtgctcctgcgggccga

SEQ ID NO: 191 - RNA, 22 nt, Homo sapiens
  source 1..22: mol_type = unassigned RNA
gctgggattacaggcatgagcc

SEQ ID NO: 192 - RNA, 20 nt, Homo sapiens
  source 1..20: mol_type = unassigned RNA
gtgagtcagggtggggctgg

SEQ ID NO: 193 - RNA, 22 nt, Homo sapiens
  source 1..22: mol_type = unassigned RNA
ctgggggtggggggctgggcgt

SEQ ID NO: 194 - RNA, 22 nt, Homo sapiens
  source 1..22: mol_type = unassigned RNA
ctgggacaggaggaggaggcag

SEQ ID NO: 195 - RNA, 20 nt, Homo sapiens
  source 1..20: mol_type = unassigned RNA
ggatccgagtcacggcacca

SEQ ID NO: 196 - RNA, 22 nt, Homo sapiens
  source 1..22: mol_type = unassigned RNA
ccaggaggcggaggaggtggag

SEQ ID NO: 197 - RNA, 18 nt, Homo sapiens
  source 1..18: mol_type = unassigned RNA
ctggcagggggagaggta

SEQ ID NO: 198 - RNA, 22 nt, Homo sapiens
  source 1..22: mol_type = unassigned RNA
tggtggaggaagagggcagctc

SEQ ID NO: 199 - RNA, 22 nt, Homo sapiens
  source 1..22: mol_type = unassigned RNA
cgtcccggggctgcgcgaggca

SEQ ID NO: 200 - RNA, 22 nt, Homo sapiens
  source 1..22: mol_type = unassigned RNA
tcaaaatcaggagtcggggctt

SEQ ID NO: 201 - RNA, 22 nt, Homo sapiens
  source 1..22: mol_type = unassigned RNA
caggaggcagtgggcgagcagg

SEQ ID NO: 202 - RNA, 19 nt, Homo sapiens
  source 1..19: mol_type = unassigned RNA
agcagggctggggattgca

SEQ ID NO: 203 - RNA, 20 nt, Homo sapiens
  source 1..20: mol_type = unassigned RNA
tgggctgctgagaaggggca

SEQ ID NO: 204 - RNA, 17 nt, Homo sapiens
  source 1..17: mol_type = unassigned RNA
gctgggcgaggctggca

SEQ ID NO: 205 - RNA, 23 nt, Homo sapiens
  source 1..23: mol_type = unassigned RNA
ttgctctgctcccccgcccccag

SEQ ID NO: 206 - RNA, 23 nt, Homo sapiens
  source 1..23: mol_type = unassigned RNA
tcaataggaaagaggtgggacct

SEQ ID NO: 207 - RNA, 17 nt, Homo sapiens
  source 1..17: mol_type = unassigned RNA
gcggggctgggcgcgcg

SEQ ID NO: 208 - RNA, 22 nt, Homo sapiens
  source 1..22: mol_type = unassigned RNA
tggggagcggcccccgggtggg

SEQ ID NO: 209 - RNA, 21 nt, Homo sapiens
  source 1..21: mol_type = unassigned RNA
tctgtggagtggggtgcctgt

SEQ ID NO: 210 - RNA, 22 nt, Homo sapiens
  source 1..22: mol_type = unassigned RNA
caggcacgggagctcaggtgag

SEQ ID NO: 211 - RNA, 25 nt, Homo sapiens
  source 1..25: mol_type = unassigned RNA
gtgaggcggggccaggagggtgtgt

SEQ ID NO: 212 - RNA, 21 nt, Homo sapiens
  source 1..21: mol_type = unassigned RNA
aagggacagggagggtcgtgg

SEQ ID NO: 213 - RNA, 20 nt, Homo sapiens
  source 1..20: mol_type = unassigned RNA
ggcggcggcggaggcggggg

SEQ ID NO: 214 - RNA, 22 nt, Homo sapiens
  source 1..22: mol_type = unassigned RNA
tcgggcctggggttgggggagc

SEQ ID NO: 215 - RNA, 19 nt, Homo sapiens
  source 1..19: mol_type = unassigned RNA
atcccaccactgccaccat

SEQ ID NO: 216 - RNA, 20 nt, Homo sapiens
  source 1..20: mol_type = unassigned RNA
ggggaggtgtgcagggctgg

SEQ ID NO: 217 - RNA, 24 nt, Homo sapiens
  source 1..24: mol_type = unassigned RNA
tgggggagccatgagataagagca

SEQ ID NO: 218 - RNA, 20 nt, Homo sapiens
  source 1..20: mol_type = unassigned RNA
gccggggctttgggtgaggg

SEQ ID NO: 219 - RNA, 22 nt, Homo sapiens
  source 1..22: mol_type = unassigned RNA
ttcagatcccagcggtgcctct

SEQ ID NO: 220 - RNA, 23 nt, Homo sapiens
  source 1..23: mol_type = unassigned RNA
tggtgggtggggaggagaagtgc

SEQ ID NO: 221 - RNA, 21 nt, Homo sapiens
  source 1..21: mol_type = unassigned RNA
tggcagggaggctgggagggg

SEQ ID NO: 222 - RNA, 22 nt, Homo sapiens
  source 1..22: mol_type = unassigned RNA
agacacatttggagagggaacc

SEQ ID NO: 223 - RNA, 18 nt, Homo sapiens
  source 1..18: mol_type = unassigned RNA
aggctgggctgggacgga

SEQ ID NO: 224 - RNA, 20 nt, Homo sapiens
  source 1..20: mol_type = unassigned RNA
tcagggagtcaggggagggc

SEQ ID NO: 225 - RNA, 23 nt, Homo sapiens
  source 1..23: mol_type = unassigned RNA
tgggcaggggcttattgtaggag

SEQ ID NO: 226 - RNA, 22 nt, Homo sapiens
  source 1..22: mol_type = unassigned RNA
tgggggaggaaggacaggccat

SEQ ID NO: 227 - RNA, 22 nt, Homo sapiens
  source 1..22: mol_type = unassigned RNA
cccctggggctgggcaggcgga

SEQ ID NO: 228 - RNA, 21 nt, Homo sapiens
  source 1..21: mol_type = unassigned RNA
ctggggggaggagaccctgct

SEQ ID NO: 229 - RNA, 17 nt, Homo sapiens
  source 1..17: mol_type = unassigned RNA
ccccgccaccgccttgg

SEQ ID NO: 230 - RNA, 20 nt, Homo sapiens
  source 1..20: mol_type = unassigned RNA
tctcttcatctaccccccag

SEQ ID NO: 231 - RNA, 23 nt, Homo sapiens
  source 1..23: mol_type = unassigned RNA
aggaggtggtactaggggccagc

SEQ ID NO: 232 - RNA, 20 nt, Homo sapiens
  source 1..20: mol_type = unassigned RNA
gcggaaggcggagcggcgga

SEQ ID NO: 233 - RNA, 22 nt, Homo sapiens
  source 1..22: mol_type = unassigned RNA
tgcggggacaggccagggcatc

SEQ ID NO: 234 - RNA, 23 nt, Homo sapiens
  source 1..23: mol_type = unassigned RNA
agggccagaggagcctggagtgg

SEQ ID NO: 235 - RNA, 22 nt, Homo sapiens
  source 1..22: mol_type = unassigned RNA
agactgacggctggaggcccat

SEQ ID NO: 236 - RNA, 20 nt, Homo sapiens
  source 1..20: mol_type = unassigned RNA
cggctctgggtctgtgggga

SEQ ID NO: 237 - RNA, 23 nt, Homo sapiens
  source 1..23: mol_type = unassigned RNA
gacacgggcgacagctgcggccc

SEQ ID NO: 238 - RNA, 23 nt, Homo sapiens
  source 1..23: mol_type = unassigned RNA
tgaggggcagagagcgagacttt

SEQ ID NO: 239 - RNA, 22 nt, Homo sapiens
  source 1..22: mol_type = unassigned RNA
gggtggggatttgttgcattac

SEQ ID NO: 240 - RNA, 22 nt, Homo sapiens
  source 1..22: mol_type = unassigned RNA
tagcagcacgtaaatattggcg

SEQ ID NO: 241 - RNA, 22 nt, Homo sapiens
  source 1..22: mol_type = unassigned RNA
aaaccgttaccattactgagtt

SEQ ID NO: 242 - RNA, 22 nt, Homo sapiens
  source 1..22: mol_type = unassigned RNA
tatagggattggagccgtggcg

SEQ ID NO: 243 - RNA, 22 nt, Homo sapiens
  source 1..22: mol_type = unassigned RNA
tcctgtactgagctgccccgag

SEQ ID NO: 244 - RNA, 18 nt, Homo sapiens
  source 1..18: mol_type = unassigned RNA
ccagaggtggggactgag

SEQ ID NO: 245 - RNA, 22 nt, Homo sapiens
  source 1..22: mol_type = unassigned RNA
agggacgggacgcggtgcagtg

SEQ ID NO: 246 - RNA, 20 nt, Homo sapiens
  source 1..20: mol_type = unassigned RNA
ccccagggcgacgcggcggg

SEQ ID NO: 247 - RNA, 21 nt, Homo sapiens
  source 1..21: mol_type = unassigned RNA
cttccgccccgccgggcgtcg

SEQ ID NO: 248 - RNA, 21 nt, Homo sapiens
  source 1..21: mol_type = unassigned RNA
aaggcagggcccccgctcccc

SEQ ID NO: 249 - RNA, 21 nt, Homo sapiens
  source 1..21: mol_type = unassigned RNA
agggccccccctcaatcctgt

SEQ ID NO: 250 - RNA, 21 nt, Homo sapiens
  source 1..21: mol_type = unassigned RNA
atcacattgccagggattacc

SEQ ID NO: 251 - RNA, 22 nt, Homo sapiens
  source 1..22: mol_type = unassigned RNA
tattgcacttgtcccggcctgt

SEQ ID NO: 252 - RNA, 25 nt, Homo sapiens
  source 1..25: mol_type = unassigned RNA
ggcggagggaagtaggtccgttggt

SEQ ID NO: 253 - RNA, 22 nt, Homo sapiens
  source 1..22: mol_type = unassigned RNA
tgggggtggtctctagccaagg

SEQ ID NO: 254 - RNA, 20 nt, Homo sapiens
  source 1..20: mol_type = unassigned RNA
gggaaaaggaagggggagga

SEQ ID NO: 255 - RNA, 21 nt, Homo sapiens
  source 1..21: mol_type = unassigned RNA
tcacctggctggcccgcccag

SEQ ID NO: 256 - RNA, 20 nt, Homo sapiens
  source 1..20: mol_type = unassigned RNA
tgggaggaggggatcttggg

SEQ ID NO: 257 - RNA, 17 nt, Homo sapiens
  source 1..17: mol_type = unassigned RNA
ggatggaggaggggtct

SEQ ID NO: 258 - RNA, 22 nt, Homo sapiens
  source 1..22: mol_type = unassigned RNA
tattgcactcgtcccggcctcc

SEQ ID NO: 259 - RNA, 22 nt, Homo sapiens
  source 1..22: mol_type = unassigned RNA
ggctggagcgagtgcagtggtg

SEQ ID NO: 260 - RNA, 21 nt, Homo sapiens
  source 1..21: mol_type = unassigned RNA
tggctgttggagggggcaggc

SEQ ID NO: 261 - RNA, 22 nt, Homo sapiens
  source 1..22: mol_type = unassigned RNA
ggggctggggccggggccgagc

SEQ ID NO: 262 - RNA, 22 nt, Homo sapiens
  source 1..22: mol_type = unassigned RNA
gggaccatcctgcctgctgtgg

SEQ ID NO: 263 - RNA, 22 nt, Homo sapiens
  source 1..22: mol_type = unassigned RNA
tggcggcggtagttatgggctt

SEQ ID NO: 264 - RNA, 23 nt, Homo sapiens
  source 1..23: mol_type = unassigned RNA
gtttgcacgggtgggccttgtct

SEQ ID NO: 265 - RNA, 21 nt, Homo sapiens
  source 1..21: mol_type = unassigned RNA
cgggggcggggccgaagcgcg

SEQ ID NO: 266 - RNA, 21 nt, Homo sapiens
  source 1..21: mol_type = unassigned RNA
cggcggggacggcgattggtc

SEQ ID NO: 267 - RNA, 17 nt, Homo sapiens
  source 1..17: mol_type = unassigned RNA
accccactcctggtacc

SEQ ID NO: 268 - RNA, 25 nt, Homo sapiens
  source 1..25: mol_type = unassigned RNA
aggggggcactgcgcaagcaaagcc

SEQ ID NO: 269 - RNA, 19 nt, Homo sapiens
  source 1..19: mol_type = unassigned RNA
ctggggagtggctggggag

SEQ ID NO: 270 - RNA, 82 nt, Homo sapiens
  source 1..82: mol_type = unassigned RNA
gggaaagcggagggcgcgcccagctcccgggctgattgcgctaacagtggccccggtgttggggcgcgtctgccgctgcccc

SEQ ID NO: 271 - RNA, 76 nt, Homo sapiens
  source 1..76: mol_type = unassigned RNA
cgcaggcctctggcggagcccattccatgccagatgctgagcgatggctggtgtgtgctgctccacaggcctggtg

SEQ ID NO: 272 - RNA, 149 nt, Homo sapiens
  source 1..149: mol_type = unassigned RNA
catcaagacccagctgagtcactgtcactgcctaccaatctcgaccggacctcgaccggctcgtctgtgttgccaatcgactcggcgtggcgtcggtcgtggtagataggcggtcatgcatacgaattttcagctcttgttctggtgac

SEQ ID NO: 273 - RNA, 54 nt, Homo sapiens
  source 1..54: mol_type = unassigned RNA
ggacaagggcggcgcgaccggcccggggctcttgggcggccgcgtttcccctcc

SEQ ID NO: 274 - RNA, 93 nt, Homo sapiens
  source 1..93: mol_type = unassigned RNA
ccttccggcgtcccaggcggggcgccgcgggaccgccctcgtgtctgtggcggtgggatcccgcggccgtgttttcctggtggcccggccatg

SEQ ID NO: 275 - RNA, 51 nt, Homo sapiens
  source 1..51: mol_type = unassigned RNA
acagaccccggggagcccggcggtgaagctcctggtatcctgggtgtctga

SEQ ID NO: 276 - RNA, 59 nt, Homo sapiens
  source 1..59: mol_type = unassigned RNA
cggggtcggcggcgacgtgctcagcttggcacccaagttctgccgctccgacgcccggc

SEQ ID NO: 277 - RNA, 84 nt, Homo sapiens
  source 1..84: mol_type = unassigned RNA
gaggctgggcggggcgcggccggatcggtcgagagcgtcctggctgatgacggtctcccgtgcccacgccccaaacgcagtctc

SEQ ID NO: 278 - RNA, 65 nt, Homo sapiens
  source 1..65: mol_type = unassigned RNA
gagggtgggcgagggcggctgagcggctccatcccccggcctgctcatccccctcgccctctcag

SEQ ID NO: 279 - RNA, 60 nt, Homo sapiens
  source 1..60: mol_type = unassigned RNA
cgctgggtccgcgcgccctgggccgggcgatgtccgcttgggggagcgaggggcggggcg

SEQ ID NO: 280 - RNA, 76 nt, Homo sapiens
  source 1..76: mol_type = unassigned RNA
gagggagctgtagagcagggagcaggaagctgtgtgtgtccagccctgacctgtcctgttctgcccccagcccctc

SEQ ID NO: 281 - RNA, 75 nt, Homo sapiens
  source 1..75: mol_type = unassigned RNA
aagcaagactgaggggcctcagaccgagcttttggaaaatagaaaagtctcgctctctgcccctcagcctaactt

SEQ ID NO: 282 - RNA, 65 nt, Homo sapiens
  source 1..65: mol_type = unassigned RNA
gggtgctcggggcaggcggctgggagcggccctcacattgatggctcctgccacctcctccgcag

SEQ ID NO: 283 - RNA, 51 nt, Homo sapiens
  source 1..51: mol_type = unassigned RNA
agagatgaagcgggggggcggggtcttgctctattgcctacgctgatctca

SEQ ID NO: 284 - RNA, 87 nt, Homo sapiens
  source 1..87: mol_type = unassigned RNA
cccaggcgcccgctcccgacccacgccgcgccgccgggtccctcctccccggagaggctgggctcgggacgcgcggctcagctcggg

SEQ ID NO: 285 - RNA, 72 nt, Homo sapiens
  source 1..72: mol_type = unassigned RNA
gatttcagtgacctggcagcagggagcgtcgtcagtgtttgactgtttatggtatgtcagggagctggttcc

SEQ ID NO: 286 - RNA, 84 nt, Homo sapiens
  source 1..84: mol_type = unassigned RNA
cggtccagacgtggcgggggtggcggcggcatcccggacggcctgtgagggatgcgccgcccactgccccgcgccgcctgaccg

SEQ ID NO: 287 - RNA, 47 nt, Homo sapiens
  source 1..47: mol_type = unassigned RNA
ctcggcgcggggcgcgggctccgggttggggcgagccaacgccgggg

SEQ ID NO: 288 - RNA, 86 nt, Homo sapiens
  source 1..86: mol_type = unassigned RNA
tgccagtctctaggtccctgagaccctttaacctgtgaggacatccagggtcacaggtgaggttcttgggagcctggcgtctggcc

SEQ ID NO: 289 - RNA, 82 nt, Homo sapiens
  source 1..82: mol_type = unassigned RNA
gtgagtgggaggccagggcacggcagggggagctgcagggctatgggaggggccccagcgtctgagccctgtcctcccgcag

SEQ ID NO: 290 - RNA, 82 nt, Homo sapiens; identical to SEQ ID NO: 289
  source 1..82: mol_type = unassigned RNA

SEQ ID NO: 291 - RNA, 74 nt, Homo sapiens
  source 1..74: mol_type = unassigned RNA
ccgcactctctccattacactaccctgcctcttctccatgagaggcagcggggtgtagtggatagagcacgggt

SEQ ID NO: 292 - RNA, 65 nt, Homo sapiens
  source 1..65: mol_type = unassigned RNA
gagggctaggtggggggcttgaagccccgagatgcctcacgtcttcacccctctcacctaagcag

SEQ ID NO: 293 - RNA, 75 nt, Homo sapiens
  source 1..75: mol_type = unassigned RNA
tggagtgggggggcaggaggggctcagggagaaagtgcatacagcccctggccctctctgcccttccgtcccctg

SEQ ID NO: 294 - RNA, 61 nt, Homo sapiens
  source 1..61: mol_type = unassigned RNA
tcggctggcgggggtagagctggctgcaggcccggcccctctcagctgctgccctctccag

SEQ ID NO: 295 - RNA, 86 nt, Homo sapiens
  source 1..86: mol_type = unassigned RNA
cgcctgagcgtgcagcaggacatcttcctgacctggtaataattaggtgagaaggatggttgggggcggtcggcgtaactcaggga

SEQ ID NO: 296 - RNA, 72 nt, Homo sapiens
  source 1..72: mol_type = unassigned RNA
gagtctgagggacccaggacaggagaaggcctatggtgatttgcattcttcctgccctggctccatcctcag

SEQ ID NO: 297 - RNA, 73 nt, Homo sapiens
  source 1..73: mol_type = unassigned RNA
tgtatccttgaatggatttttggagcaggagtggacacctgacccaaaggaaatcaatccataggctagcaat

SEQ ID NO: 298 - RNA, 70 nt, Homo sapiens
  source 1..70: mol_type = unassigned RNA
ctcgggcccgaccgcgccggcccgcacctcccggcccggagctgcgggctgcggtcagggcgatcccggg

SEQ ID NO: 299 - RNA, 69 nt, Homo sapiens
  source 1..69: mol_type = unassigned RNA
gggcttagggatgggaggccaggatgaagattaatccctaatccccaacactggccttgctatccccag

SEQ ID NO: 300 - RNA, 63 nt, Homo sapiens
  source 1..63: mol_type = unassigned RNA
accctagggtggggctggaggtggggctgaggctgagtcttcctccccttcctccctgcccag

SEQ ID NO: 301 - RNA, 105 nt, Homo sapiens
  source 1..105: mol_type = unassigned RNA
gcgggcggcggcggcggcagcagcagcaggtgcggggcggcggccgcgctggccgctcgactccgcagctgctcgttctgcttctccagcttgcgcaccagctcc

SEQ ID NO: 302 - RNA, 63 nt, Homo sapiens
  source 1..63: mol_type = unassigned RNA
ggctccgcagggccctggcgcaggcatccagacagcgggcgaatgcctcccccggccccgcag

SEQ ID NO: 303 - RNA, 74 nt, Homo sapiens
  source 1..74: mol_type = unassigned RNA
gtgcgtggtggctcgaggcgggggtgggggcctcgccctgcttgggccctccctgacctctccgctccgcacag

SEQ ID NO: 304 - RNA, 62 nt, Homo sapiens
  source 1..62: mol_type = unassigned RNA
tggcctagggggcggcttgtggagtgtatgggctgagccttgctctgctcccccgcccccag

SEQ ID NO: 305 - RNA, 67 nt, Homo sapiens
  source 1..67: mol_type = unassigned RNA
gtgggaggggagaggcagcaagcacacagggcctgggactagcatgctgacctccctcctgccccag

SEQ ID NO: 306 - RNA, 61 nt, Homo sapiens
  source 1..61: mol_type = unassigned RNA
gggggcgggagctggggtctgcaggttcgcactgatgcctgctcgccctgtctcccgctag

SEQ ID NO: 307 - RNA, 75 nt, Homo sapiens
  source 1..75: mol_type = unassigned RNA
ggctgtgccgggtagagagggcagtgggaggtaagagctcttcacccttcaccaccttctccacccagcatggcc

SEQ ID NO: 308 - RNA, 89 nt, Homo sapiens
  source 1..89: mol_type = unassigned RNA
gccggcgcccgagctctggctccgtgtcttcactcccgtgcttgtccgaggagggagggagggacgggggctgtgctggggcagctgga

SEQ ID NO: 309 - RNA, 61 nt, Homo sapiens
  source 1..61: mol_type = unassigned RNA
gtgcggaacgctggccggggcgggaggggaagggacgcccggccggaacgccgcactcacg

SEQ ID NO: 310 - RNA, 70 nt, Homo sapiens
  source 1..70: mol_type = unassigned RNA
aaaagcctgtccctaagtccctcccagccttccagagttggtgccaggaaggatttagggacaggctttg

SEQ ID NO: 311 - RNA, 67 nt, Homo sapiens
  source 1..67: mol_type = unassigned RNA
gtgaggaggggctggcagggacccctccaagttggggacggcagccagcccctgctcacccctcgcc

SEQ ID NO: 312 - RNA, 94 nt, Homo sapiens
  source 1..94: mol_type = unassigned RNA
cgggcagcgggtgccaggcacggtgtcagcaggcaacatggccgagaggccggggcctccgggcggcgccgtgtccgcgaccgcgtaccctgac

SEQ ID NO: 313 - RNA, 92 nt, Homo sapiens
  source 1..92: mol_type = unassigned RNA
cctgtccctcctgccctgcgcctgcccagccctcctgctctggtgactgaggaccgccaggcaggggctggtgctgggcggggggcggcggg

SEQ ID NO: 314 - RNA, 94 nt, Homo sapiens
  source 1..94: mol_type = unassigned RNA
aattcagccctgccactggcttatgtcatgaccttgggctactcaggctgtctgcacaatgagccagttggacaggagcagtgccactcaactc

SEQ ID NO: 315 - RNA, 60 nt, Homo sapiens
  source 1..60: mol_type = unassigned RNA
ggtgcctcgggagggcatgggccaggccacataatgagccaaacccctgtctacccgcag

SEQ ID NO: 316 - RNA, 92 nt, Homo sapiens
  source 1..92: mol_type = unassigned RNA
gtcagtgtctgggcggacagctgcaggaaagggaagaccaaggcttgctgtctgtccagtctgccaccctaccctgtctgttcttgccacag

SEQ ID NO: 317 - RNA, 80 nt, Homo sapiens
  source 1..80: mol_type = unassigned RNA
cgggaatgccgcggcggggacggcgattggtccgtatgtgtggtgccaccggccgccggctccgccccggcccccgcccc

SEQ ID NO: 318 - RNA, 84 nt, Homo sapiens
  source 1..84: mol_type = unassigned RNA
ctccccatggccctgtctcccaacccttgtaccagtgctgggctcagaccctggtacaggcctgggggacagggacctggggac

SEQ ID NO: 319 - RNA, 106 nt, Homo sapiens
  source 1..106: mol_type = unassigned RNA
agaagaatgcccaaccagccctcagttgctacagttccctgttgtttcagctcgacaacaacaggcggctgtagcaatggggggctggatgggcatctcaatgtgc

SEQ ID NO: 320 - RNA, 79 nt, Homo sapiens
  source 1..79: mol_type = unassigned RNA
gtgcagatccttgggagccctgttagactctggattttacacttggagtgaacgggcgccatcccgaggctttgcacag

SEQ ID NO: 321 - RNA, 102 nt, Homo sapiens
  source 1..102: mol_type = unassigned RNA
gcttatcgaggaaaagatcgaggtgggttggggcgggctctggggatttggtctcacagcccggatcccagcccacttaccttggttactctccttccttct

SEQ ID NO: 322 - RNA, 90 nt, Homo sapiens
  source 1..90: mol_type = unassigned RNA
cgggcggggcgggtccggccgcctccgagcccggccggcagcccccggccttaaagcgcgggctgtccggaggggtcggctttcccaccg

SEQ ID NO: 323 - RNA, 59 nt, Homo sapiens
  source 1..59: mol_type = unassigned RNA
ggggccaggcagggaggtgggaccatgggggccttgctgtgtgaccaccgttcctgcag

SEQ ID NO: 324 - RNA, 62 nt, Homo sapiens
  source 1..62: mol_type = unassigned RNA
gtgagtagtggcgcgcggcggctcggagtacctctgccgccgcgcgcatcggctcagcatgc

SEQ ID NO: 325 - RNA, 85 nt, Homo sapiens
  source 1..85: mol_type = unassigned RNA
tctgaggtacccggggcagattggtgtagggtgcaaagcctgcccgccccctaagccttctgcccccaactccagcctgtcagga

SEQ ID NO: 326 - RNA, 96 nt, Homo sapiens
  source 1..96: mol_type = unassigned RNA
ctcgggaggggcgggaggggggtccccggtgctcggatctcgagggtgcttattgttcggtccgagcctgggtctccctcttccccccaacccccc

SEQ ID NO: 327 - RNA, 80 nt, Homo sapiens
  source 1..80: mol_type = unassigned RNA
gcgtcaagatggcggcggggaggtaggcagagcaggacgccgctgctgccgccgccaccgccgcctccgctccagtcgcc

SEQ ID NO: 328 - RNA, 82 nt, Homo sapiens
  source 1..82: mol_type = unassigned RNA
tgagctgttggattcggggccgtagcactgtctgagaggtttacatttctcacagtgaaccggtctctttttcagctgcttc

SEQ ID NO: 329 - RNA, 83 nt, Homo sapiens
  source 1..83: mol_type = unassigned RNA
gtgagtgggagccccagtgtgtggttggggccatggcgggtgggcagcccagcctctgagccttcctcgtctgtctgccccag

SEQ ID NO: 330 - RNA, 87 nt, Homo sapiens
  source 1..87: mol_type = unassigned RNA
accgcagggaaaatgagggacttttgggggcagatgtgtttccattccactatcataatgcccctaaaaatccttattgctcttgca

SEQ ID NO: 331 - RNA, 110 nt, Homo sapiens
  source 1..110: mol_type = unassigned RNA
ggctacagtctttcttcatgtgactcgtggacttccctttgtcatcctatgcctgagaatatatgaaggaggctgggaaggcaaagggacgttcaattgtcatcactggc

SEQ ID NO: 332 - RNA, 80 nt, Homo sapiens
  source 1..80: mol_type = unassigned RNA
ctgcagcgtgcttctccaggccccgcgcgcggacagacacacggacaagtcccgccaggggctgggcgcgcgccagccgg

SEQ ID NO: 333 - RNA, 81 nt, Homo sapiens
  source 1..81: mol_type = unassigned RNA
ctccctgggagggcgtggatgatggtgggagaggagccccactgtggaagtctgacccccacatcgccccaccttccccag

SEQ ID NO: 334 - RNA, 67 nt, Homo sapiens
  source 1..67: mol_type = unassigned RNA
cctgcaggcagaagtggggctgacagggcagagggttgcgccccctcaccatcccttctgcctgcag

SEQ ID NO: 335 - RNA, 67 nt, Homo sapiens; identical to SEQ ID NO: 334
  source 1..67: mol_type = unassigned RNA

SEQ ID NO: 336 - RNA, 67 nt, Homo sapiens; identical to SEQ ID NO: 334
  source 1..67: mol_type = unassigned RNA

SEQ ID NO: 337 - RNA, 67 nt, Homo sapiens; identical to SEQ ID NO: 334
  source 1..67: mol_type = unassigned RNA

SEQ ID NO: 338 - RNA, 75 nt, Homo sapiens
  source 1..75: mol_type = unassigned RNA
gtcagaggggggatgtgcatgctggttggggtgggctgcctgtggaccaatcagcgtgcacttccccaccctgaa

SEQ ID NO: 339 - RNA, 80 nt, Homo sapiens
  source 1..80: mol_type = unassigned RNA
tgagaggccgcaccttgccttgctgcccgggccgtgcacccgtgggccccagggcgacgcggcgggggcggccctagcga

SEQ ID NO: 340 - RNA, 153 nt, Homo sapiens
  source 1..153: mol_type = unassigned RNA
gctccgccccacgtcgcatgcgccccgggaacgcgtggggcggagcttccggaggccccgctctgctgccgaccctgtggagcggagggtgaagcctccggatgccagtccctcatcgctggcctggtcgcgctgtggcgaagggggcggagc

SEQ ID NO: 341 - RNA, 153 nt, Homo sapiens
  source 1..153: mol_type = unassigned RNA
gctccgccccacgtcgcatgcgccccgggaacgcgtggggcggagcttccggaggccccgccctgctgccgaccctgtggagcggagggtgaagcctccggatgccagtccctcatcgctggcccggtcgcgctgtggcgaagggggcggagc

SEQ ID NO: 342 - RNA, 66 nt, Homo sapiens
  source 1..66: mol_type = unassigned RNA
cagagcagggcagggaaggtgggagaggggcccagctgaccctcctgtcacccgctccttgcccag

SEQ ID NO: 343 - RNA, 119 nt, Homo sapiens
  source 1..119: mol_type = unassigned RNA
agcctgcgccggagccggggcctgagcccgggccgcgcaggccgtgaactcgtcgagctgcgcgtgcggccggtgctcaacctgccgggtcctggccccgcgctcccgcgcgccctgga

SEQ ID NO: 344 - RNA, 63 nt, Homo sapiens
  source 1..63: mol_type = unassigned RNA
cttgcccgggagaaggaggtggcctggagagctgctgtctccagccgccgcctgtctccacag

SEQ ID NO: 345 - RNA, 76 nt, Homo sapiens
  source 1..76: mol_type = unassigned RNA
actgactttgagtctctcctcagggtgctgcaggcaaagctggggacccagggagagacgtaagtgaggggagatg

SEQ ID NO: 346 - RNA, 115 nt, Homo sapiens
  source 1..115: mol_type = unassigned RNA
ggtgccgagggccgtccggcatcctaggcgggtcgctgcggtacctccctcctgtctgtggcggtgggatcccgtggccgtgttttcctggtggcccggccgtgcctgaggtttc

SEQ ID NO: 347 - RNA, 80 nt, Homo sapiens
  source 1..80: mol_type = unassigned RNA
ggttccggagccccggcgcgggcgggttctggggtgtagacgctgctggccagcccgccccagccgaggttctcggcacc

SEQ ID NO: 348 - RNA, 69 nt, Homo sapiens
  source 1..69: mol_type = unassigned RNA
ccgggcaggcaggtgtagggtggagcccactgtggctcctgactcagccctgctgccttcacctgccag

SEQ ID NO: 349 - RNA, 77 nt, Homo sapiens
  source 1..77: mol_type = unassigned RNA
catgagaaatcctgctggtcaaccatagccctggtcagactctccggggctgtgattgaccagcaggacttctcatg

SEQ ID NO: 350 - RNA, 68 nt, Homo sapiens
  source 1..68: mol_type = unassigned RNA
gactcggctgcggtggacaagtccggctccagaacctggacaccgctcagccggccgcggcaggggtc

SEQ ID NO: 351 - RNA, 73 nt, Homo sapiens
  source 1..73: mol_type = unassigned RNA
cggcgacggcggggtgggtgaggtcgggccccaagactcggggtttgccgggcgcctcagttcaccgcggccg

SEQ ID NO: 352 - RNA, 49 nt, Homo sapiens
  source 1..49: mol_type = unassigned RNA
ggtgaggcgggggggcgagccctgaggggctctcgcttctggcgccaag

SEQ ID NO: 353 - RNA, 79 nt, Homo sapiens
  source 1..79: mol_type = unassigned RNA
ctcgaggtgctgggggacgcgtgagcgcgagccgcttcctcacggctcggccgcggcgcgtagcccccgccacatcggg

SEQ ID NO: 354 - RNA, 71 nt, Homo sapiens
  source 1..71: mol_type = unassigned RNA
ggtgagtgggagccggtggggctggagtaagggcacgcccggggctgccccacctgctgaccaccctcccc

SEQ ID NO: 355 - RNA, 65 nt, Homo sapiens
  source 1..65: mol_type = unassigned RNA
gagaatggggggacagatggagaggacacaggctggcactgaggtcccctccactttcctcctag

SEQ ID NO: 356 - RNA, 79 nt, Homo sapiens
  source 1..79: mol_type = unassigned RNA
gtgaggtgggggccagcagggagtgggctgggctgggctgggccaaggtacaaggcctcaccctgcatcccgcacccag

SEQ ID NO: 357 - RNA, 80 nt, Homo sapiens
  source 1..80: mol_type = unassigned RNA
catccaggacaatggtgagtgccggtgcctgccctggggccgtccctgcgcaggggccgggtgctcaccgcatctgcccc

SEQ ID NO: 358 - RNA, 61 nt, Homo sapiens
  source 1..61: mol_type = unassigned RNA
tctcgtttgatctcggaagctaagcagggttgggcctggttagtacttggatgggaaactt

SEQ ID NO: 359 - RNA, 53 nt, Homo sapiens
  source 1..53: mol_type = unassigned RNA
gtttgatctcggaagctaagcagggtcgggcctggttagtacttggatgggag

SEQ ID NO: 360 - RNA, 92 nt, Homo sapiens
  source 1..92: mol_type = unassigned RNA
cgctgcgcttctgggcccgcggcgggcgtggggctgcccgggccggtcgaccagcgcgccgtagctcccgaggcccgagccgcgacccgcgg

SEQ ID NO: 361 - RNA, 84 nt, Homo sapiens
  source 1..84: mol_type = unassigned RNA
gctggcgtcggtgctggggagcggcccccgggtgggcctctgctctggcccctcctggggcccgcactctcgctctgggcccgc

SEQ ID NO: 362 - RNA, 79 nt, Homo sapiens
  source 1..79: mol_type = unassigned RNA
cagcctggggaaggcttggcagggaagacacatgagcagtgcctccacttcacgcctctcccttgtctcctttccctag

SEQ ID NO: 363 - RNA, 83 nt, Homo sapiens
  source 1..83: mol_type = unassigned RNA
gggtttcctctgcctttttttccaatgaaaataacgaaacctgttatttcccattgagggggaaaaaggcgggagaagcccca

SEQ ID NO: 364 - RNA, 60 nt, Homo sapiens
  source 1..60: mol_type = unassigned RNA
gagcaggcgaggctgggctgaacccgtgggtgaggagtgcagcccagctgaggcctctgc

SEQ ID NO: 365 - RNA, 73 nt, Homo sapiens
  source 1..73: mol_type = unassigned RNA
agggagaaaagctgggctgagaggcgactggtgtctaatttgtttgtctctccaactcagactgcctggccca

SEQ ID NO: 366 - RNA, 88 nt, Homo sapiens
  source 1..88: mol_type = unassigned RNA
gtggggccaggcggtggtgggcactgctggggtgggcacagcagccatgcagagcgggcatttgaccccgtgccacccttttccccag

SEQ ID NO: 367 - RNA, 90 nt, Homo sapiens
  source 1..90: mol_type = unassigned RNA
gtgtctctctggagaccctgcagccttcccacccaccagggagctttccatgggctgtggggaaggcgtcagtgtcgggtgagggaacac

SEQ ID NO: 368 - RNA, 64 nt, Homo sapiens
  source 1..64: mol_type = unassigned RNA
gaggcactgggtaggtggggctccagggctcctgacacctggacctctcctccccaggcccaca

SEQ ID NO: 369 - RNA, 64 nt, Homo sapiens
  source 1..64: mol_type = unassigned RNA
tgaggatggggtgagatggggaggagcagccagtcctgtctcaccgctcttcccctgaccccag

SEQ ID NO: 370 - RNA, 73 nt, Homo sapiens
  source 1..73: mol_type = unassigned RNA
ggcgaggggaggcgcaggctcggaaaggcgcgcgaggctccaggctccttcccgatccaccgctctcctcgct

SEQ ID NO: 371 - RNA, 81 nt, Homo sapiens
  source 1..81: mol_type = unassigned RNA
tacaggtgcaggggaactgtagatgaaaaggcttggcacttgagggaaagcctcagttcattctcattttgctcacctgtt

SEQ ID NO: 372 - RNA, 68 nt, Homo sapiens
  source 1..68: mol_type = unassigned RNA
gaatggaagaagaaggcggtcggtctgcgggagccaggccgcagagccatccgccttctgtccatgtc

SEQ ID NO: 373 - RNA, 82 nt, Homo sapiens
  source 1..82: mol_type = unassigned RNA
gctacggggagcggggaggaagtgggcgctgcttctgcgttatctggaaggagcagcccactcctgtcctgggctctgtggt

SEQ ID NO: 374 - RNA, 89 nt, Homo sapiens
  source 1..89: mol_type = unassigned RNA
acctccgggacggctgggcgccggcggccgggagatccgcgcttcctgaatcccggccggcccgcccggcgcccgtccgcccgcgggtc

SEQ ID NO: 375 - RNA, 63 nt, Homo sapiens
  source 1..63: mol_type = unassigned RNA
gagtcgaggactggtggaagggcctttcccctcagaccaaggccctggccccagcttcttctc

SEQ ID NO: 376 - RNA, 64 nt, Homo sapiens
  source 1..64: mol_type = unassigned RNA
tgctctgtaggcatgaggcagggcccaggttccatgtgatgctgaagctctgacattcctgcag

SEQ ID NO: 377 - RNA, 109 nt, Homo sapiens
  source 1..109: mol_type = unassigned RNA
ggtcgggctcaccatgacacagtgtgagacctcgggctacaacacaggacccgggcgctgctctgacccctcgtgtcttgtgttgcagccggagggacgcaggtccgca

SEQ ID NO: 378 - RNA, 94 nt, Homo sapiens
  source 1..94: mol_type = unassigned RNA
cagtgcgacgggcggagcttccagacgctccgccccacgtcgcatgcgccccgggaaagcgtggggcggagcttccggaggccccgccctgctg

SEQ ID NO: 379 - RNA, 88 nt, Homo sapiens
  source 1..88: mol_type = unassigned RNA
gcgacgggcggagcttccagacgctccgccccacgtcgcatgcgccccgggaaagcgtggggcggagcttccggaggccccgccctgc

SEQ ID NO: 380 - RNA, 94 nt, Homo sapiens; identical to SEQ ID NO: 378
  source 1..94: mol_type = unassigned RNA

SEQ ID NO: 381 - RNA, 70 nt, Homo sapiens
  source 1..70: mol_type = unassigned RNA
gtccctgggggctgggatgggccatggtgtgctctgatccccctgtggtctcttggcccccaggaactcc

SEQ ID NO: 382 - RNA, 62 nt, Homo sapiens
  source 1..62: mol_type = unassigned RNA
ccttctgcggcagagctggggtcaccagccctcatgtacttgtgacttctcccctgccacag

SEQ ID NO: 383 - RNA, 82 nt, Homo sapiens
  source 1..82: mol_type = unassigned RNA
acctgtaggtgacagtcaggggcggggtgtggtggggctggggctggccccctcctcacacctctcctggcatcgcccccag

SEQ ID NO: 384 - RNA, 73 nt, Homo sapiens
  source 1..73: mol_type = unassigned RNA
ctggtgtttgaggcgatgtggggatgtagagacaacttcccagtctcatttcctcatcctgccaggccaccat

SEQ ID NO: 385 - RNA, 68 nt, Homo sapiens
  source 1..68: mol_type = unassigned RNA
agggttggggggacaggatgagaggctgtcttcattccctcttgaccacccctcgtttcttcccccag

SEQ ID NO: 386 - RNA, 61 nt, Homo sapiens
  source 1..61: mol_type = unassigned RNA
gagggcagcgtgggtgtggcggaggcaggcgtgaccgtttgccgccctctcgctgctctag

SEQ ID NO: 387 - RNA, 72 nt, Homo sapiens
  source 1..72: mol_type = unassigned RNA
tctcctcgaggggtctctgcctctacccaggactctttcatgaccaggaggctgaggcccctcacaggcggc

SEQ ID NO: 388 - RNA, 73 nt, Homo sapiens
  source 1..73: mol_type = unassigned RNA
gttcaagtgggaggacaggaggcaggtgtggttggaggaagcagcctgaacctgcctccctgacattccacag

SEQ ID NO: 389 - RNA, 97 nt, Homo sapiens
  source 1..97: mol_type = unassigned RNA
cccgggaccttggtccaggcgctggtctgcgtggtgctcgggtggataagtctgatctgagcaccacacaggccgggcgccgggaccaagggggctc

SEQ ID NO: 390 - RNA, 70 nt, Homo sapiens
  source 1..70: mol_type = unassigned RNA
ggtttctccttgaggagacatggtgggggccggtcaggcagcccatgccatgtgtcctcatggagaggcc

SEQ ID NO: 391 - RNA, 110 nt, Homo sapiens
  source 1..110: mol_type = unassigned RNA
tcacctggccatgtgacttgtgggcttccctttgtcatccttcgcctagggctctgagcagggcagggacagcaaaggggtgctcagttgtcacttcccacagcacggag

SEQ ID NO: 392 - RNA, 85 nt, Homo sapiens
  source 1..85: mol_type = unassigned RNA
gggacggggcctgcaggcagaagtggggctgacagggcagagggttgcgccccctcaccaccccttctgcctgcagcggtgggct

SEQ ID NO: 393 - RNA, 71 nt, Homo sapiens
  source 1..71: mol_type = unassigned RNA
ggggcctgcaggcagaagtggggctgacagggcagagggttgcgccccctcaccaccccttctgcctgcag

SEQ ID NO: 394 - RNA, 56 nt, Homo sapiens
  source 1..56: mol_type = unassigned RNA
cgctcgggcggaggtggttgagtgccgactggcgcctgacccaccccctcccgcag

SEQ ID NO: 395 - RNA, 89 nt, Homo sapiens
  source 1..89: mol_type = unassigned RNA
agcctgtgggaaagagaagagcagggcagggtgaaggcccggcggagacactctgcccaccccacaccctgcctatgggccacacagct

SEQ ID NO: 396 - RNA, 90 nt, Homo sapiens
  source 1..90: mol_type = unassigned RNA
tctaagaaacgcagtggtctctgaagcctgcaggggcaggccagccctgcactgaacgcctgttcttgccaggtggcagaaggttgctgc

SEQ ID NO: 397 - RNA, 86 nt, Homo sapiens
  source 1..86: mol_type = unassigned RNA
ggggctgggggtgtggggagagagagtgcacagccagctcagggattaaagctctttctctctctctctctcccacttccctgcag

SEQ ID NO: 398 - RNA, 98 nt, Homo sapiens
  source 1..98: mol_type = unassigned RNA
ttgggcaaggtgcggggctagggctaacagcagtcttactgaaggtttcctggaaaccacgcacatgctgttgccactaacctcaaccttactcggtc

SEQ ID NO: 399 - RNA, 73 nt, Homo sapiens
  source 1..73: mol_type = unassigned RNA
aggccaggtgggtatggaggagccctcatatggcagttggcgagggcccagtgagcccctctctgctctccag

SEQ ID NO: 400 - RNA, 74 nt, Homo sapiens
  source 1..74: mol_type = unassigned RNA
gcagcccggtgagcgctcgctggcctggcagtgcgtcggaagaacagggcgggtggggccgcgcacatctctgc

SEQ ID NO: 401 - RNA, 55 nt, Homo sapiens
  source 1..55: mol_type = unassigned RNA
gggggctggggcgcggggaggtgctaggtcggcctcggctcccgcgccgcacccc

SEQ ID NO: 402 - RNA, 67 nt, Homo sapiens
  source 1..67: mol_type = unassigned RNA
ggcagccagggggatgggcgagcttgggcccattcctttccttaccctaccccccatccccctgtag

SEQ ID NO: 403 - RNA, 64 nt, Homo sapiens
  source 1..64: mol_type = unassigned RNA
aaccccgggccggaggtcaagggcgtcgcttctccctaatgttgcctcttttccacggcctcag

SEQ ID NO: 404 - RNA, 68 nt, Homo sapiens
  source 1..68: mol_type = unassigned RNA
ctcaggctcagtggtgcatgcttatagtcccagccactctggaggctgaaggaagatggcttgagcct

SEQ ID NO: 405 - RNA, 67 nt, Homo sapiens
  source 1..67: mol_type = unassigned RNA
ctggtccatttccctgccattcccttggcttcaatttactcccagggctggcagtgacatgggtcaa

SEQ ID NO: 406 - RNA, 101 nt, Homo sapiens
  source 1..101: mol_type = unassigned RNA
gatgggccccttgtgtcctgaattgggtgggggctctgagtggggaaagtgggggcctaggggaggtcacagttgggtctaggggtcaggagggcccagga

SEQ ID NO: 407 - RNA, 137 nt, Homo sapiens
  source 1..137: mol_type = unassigned RNA
agccagacaagagggtcatggggagtcactgtcaacccagagcaggcactgcccctgcgaccagcctggggcatcggttggggtgcaggggtctgctggtgatgctttccatctctttgctttgtcctgattgtagc

SEQ ID NO: 408 - RNA, 95 nt, Homo sapiens
  source 1..95: mol_type = unassigned RNA
gacaccacatgctcctccaggcctgcctgccctccaggtcatgttccagtgtcccacagatgcagcaccacggcccaggcggcattggtgtcacc

SEQ ID NO: 409 - RNA, 80 nt, Homo sapiens
  source 1..80: mol_type = unassigned RNA
aggacccttccagagggccccccctcaatcctgttgtgcctaattcagagggttgggtggaggctctcctgaagggctct

SEQ ID NO: 410 - RNA, 93 nt, Homo sapiens
  source 1..93: mol_type = unassigned RNA
gtaaggagggggatgaggggtcatatctcttctcagggaaagcaggagcccttcagcagggtcagggcccctcatcttcccctcctttcccag

SEQ ID NO: 411 - RNA, 62 nt, Homo sapiens
  source 1..62: mol_type = unassigned RNA
gtgagtggggctcccgggacggcgcccgccctggccctggcccggcgacgtctcacggtccc

SEQ ID NO: 412 - RNA, 69 nt, Homo sapiens
  source 1..69: mol_type = unassigned RNA
gaacctcggggcatgggggagggaggctggacaggagagggctcacccaggccctgtcctctgccccag

SEQ ID NO: 413 - RNA, 60 nt, Homo sapiens
  source 1..60: mol_type = unassigned RNA
caaggtgggggagatgggggttgaacttcatttctcatgctcatccccatctcctttcag

SEQ ID NO: 414 - RNA, 75 nt, Homo sapiens
  source 1..75: mol_type = unassigned RNA
gtagttgttctacagaagacctggatgtgtaggagctaagacacactccaggggagctgtggaagcagtaacacg

SEQ ID NO: 415 - RNA, 56 nt, Homo sapiens
  source 1..56: mol_type = unassigned RNA
cgaccgcacccgcccgaagctgggtcaaggagcccagcaggacgggagcgcggcgc

SEQ ID NO: 416 - RNA, 61 nt, Homo sapiens
  source 1..61: mol_type = unassigned RNA
gagggttggggtggagggccaaggagctgggtggggtgccaagcctctgtccccaccccag

SEQ ID NO: 417 - RNA, 66 nt, Homo sapiens
  source 1..66: mol_type = unassigned RNA
gggcatggggaggtgtggagtcagcatggggctaggaggccccgcgctgacccgccttctccgcag

SEQ ID NO: 418 - RNA, 103 nt, Homo sapiens
  source 1..103: mol_type = unassigned RNA
gtgtcggctgtggcgtgactgtccctctgtgtcccccactaggcccactgctcagtggagcgtggaggacgaggaggaggccgtccacgagcaatgccagcat

SEQ ID NO: 419 - RNA, 109 nt, Homo sapiens
  source 1..109: mol_type = unassigned RNA
tcccgcattccctctgctttggtcaggtggtgccctccttccatgggtagagccagagatggtgggttctggctggtcagatgggagtggacagagacccggggtcctc

SEQ ID NO: 420 - RNA, 81 nt, Homo sapiens
  source 1..81: mol_type = unassigned RNA
catcctccttacgtcccaccccccactcctgtttctggtgaaatattcaaacaggagtgggggtgggacataaggaggata

SEQ ID NO: 421 - RNA, 73 nt, Homo sapiens
  source 1..73: mol_type = unassigned RNA
gtgggcgggggcaggtgtgtggtgggtggtggcctgcggtgagcagggccctcacacctgcctcgccccccag

SEQ ID NO: 422 - RNA, 71 nt, Homo sapiens
  source 1..71: mol_type = unassigned RNA
gggtaaaggggcagggacgggtggccccaggaagaagggcctggtggagccgctcttctccctgcccacag

SEQ ID NO: 423 - RNA, 52 nt, Homo sapiens
  source 1..52: mol_type = unassigned RNA
tagccgggcgtggtggtgggggcctgtggtcccagctactttggaggctgag

SEQ ID NO: 424 - RNA, 93 nt, Homo sapiens
  source 1..93: mol_type = unassigned RNA
ggcgcttttgtgcgcgcccgggtctgttggtgctcagagtgtggtcaggcggctcggactgagcaggtgggtgcggggctcggaggaggcggc

SEQ ID NO: 425 - RNA, 113 nt, Homo sapiens
  source 1..113: mol_type = unassigned RNA
gccgggtggggcggggcggcctcaggaggggcccagctcccctggatgtgctgcggtggggccggaggggcgtcacgtgcacccaagtgacgccccttctgattctgcctcag

SEQ ID NO: 426 - RNA, 84 nt, Homo sapiens
  source 1..84: mol_type = unassigned RNA
ggcccctccttctcagccccagctcccgctcacccctgccacgtcaaaggaggcagaaggggagttgggagcagagaggggacc

SEQ ID NO: 427 - RNA, 100 nt, Homo sapiens
  source 1..100: mol_type = unassigned RNA
gtgagcgggcgcggcagggatcgcgggcgggtggcggcctagggcgcggagggcggaccgggaatggcgcgccgtgcgccgccggcgtaactgcggcgct

SEQ ID NO: 428 - RNA, 63 nt, Homo sapiens
  source 1..63: mol_type = unassigned RNA
aatgggtgggtgctggtgggagccgtgccctggccactcattcggctctctccctcaccctag

SEQ ID NO: 429 - RNA, 59 nt, Homo sapiens
  source 1..59: mol_type = unassigned RNA
ctgtgtcggggagtctggggtccggaattctccagagcctctgtgcccctacttcccag

SEQ ID NO: 430 - RNA, 71 nt, Homo sapiens
  source 1..71: mol_type = unassigned RNA
tgaccacccccgggcaaagacctgcagatcccctgttagagacgggcccaggactttgtgcggggtgccca

SEQ ID NO: 431 - RNA, 54 nt, Homo sapiens
  source 1..54: mol_type = unassigned RNA
agcagcaggggagagagaggagtcctctagacaccgactctgtctcctgcagat

SEQ ID NO: 432 - RNA, 85 nt, Homo sapiens
  source 1..85: mol_type = unassigned RNA
ggcgcctcctgctctgctgtgccgccagggcctcccctagcgcgccttctggagaggctttgtgcggatacggggctggaggcct

SEQ ID NO: 433 - RNA, 96 nt, Homo sapiens
  source 1..96: mol_type = unassigned RNA
agatgtgctctcctggcccatgaaatcaagcgtgggtgagacctggtgcagaacgggaaggcgacccatacttggtttcagaggctgtgagaataa

SEQ ID NO: 434 - RNA, 100 nt, Homo sapiens
  source 1..100: mol_type = unassigned RNA
agctcagggcggctgcgcagagggctggactcagcggcggagctggctgctggcctcagttctgcctctgtccaggtccttgtgacccgcccgctctcct

SEQ ID NO: 435 - RNA, 50 nt, Homo sapiens
  source 1..50: mol_type = unassigned RNA
acccgggcgtggtggtgggggtgggtgcctgtaattccagctagttggga

SEQ ID NO: 436 - RNA, 80 nt, Homo sapiens
  source 1..80: mol_type = unassigned RNA
tgccgtcggcctggggaggaggaagggcaagtccaaaggtatacagttggtctgttcattctctctttttggcctacaag

SEQ ID NO: 437 - RNA, 63 nt, Homo sapiens
  source 1..63: mol_type = unassigned RNA
gaggtgtaggggaggttgggccagggatgccttcactgtgtctctctggtcttgccaccccag

SEQ ID NO: 438 - RNA, 60 nt, Homo sapiens
  source 1..60: mol_type = unassigned RNA
aggcctagggggtggcaggctggccatcagtgtgggctaaccctgtcctctccctcccag

SEQ ID NO: 439 - RNA, 67 nt, Homo sapiens
  source 1..67: mol_type = unassigned RNA
gtggcactcaaactgtgggggcactttctgctctctggtgaaagtgccgccatcttttgagtgttac

SEQ ID NO: 440 - RNA, 68 nt, Homo sapiens
  source 1..68: mol_type = unassigned RNA
gggcgcagggggactgggggtgagcaggcccagaacccagctcgtgctcactctcagtccctccctag

SEQ ID NO: 441 - RNA, 64 nt, Homo sapiens
  source 1..64: mol_type = unassigned RNA
gagctctgggaggggctgggtttggcaggacagtttccaagccctgtctcctcccatcttccag

SEQ ID NO: 442 - RNA, 67 nt, Homo sapiens
  source 1..67: mol_type = unassigned RNA
aaatctctctccatatctttcctgcagcccccaggtgggggggaagaaaaggtggggaattagattc

SEQ ID NO: 443 - RNA, 115 nt, Homo sapiens
  source 1..115: mol_type = unassigned RNA
gagcaaaaaccagagaacaacatgggagcgttcctaacccctaaggcaactggatgggagacctgacccatccagttctctgagggggctcttgtgtgttctacaaggttgttca

SEQ ID NO: 444 - RNA, 86 nt, Homo sapiens
  source 1..86: mol_type = unassigned RNA
agagccggggccatggagcagcctgtgtagacggggacctgccctgcatgggcaccccctcactggctgcttcccttggtctccag

SEQ ID NO: 445 - RNA, 82 nt, Homo sapiens
  source 1..82: mol_type = unassigned RNA
tgtgggcagggccctggggagctgaggctctgggggtggccggggctgaccctgggcctctgctccccagtgtctgaccgcg

SEQ ID NO: 446 - RNA, 136 nt, Homo sapiens
  source 1..136: mol_type = unassigned RNA
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 447 - RNA, 66 nt, Homo sapiens
  source 1..66: mol_type = unassigned RNA
tcaagacggggagtcaggcagtggtggagatggagagccctgagcctccactctcctggcccccag

SEQ ID NO: 448 - RNA, 78 nt, Homo sapiens
  source 1..78: mol_type = unassigned RNA
ggcctcaggcaggcgcacccgaccacatgcatggctggtggcggcgtgcaggggtcgggtgggccaggctgtggggcg

SEQ ID NO: 449 - RNA, 69 nt, Homo sapiens
  source 1..69: mol_type = unassigned RNA
ctttcggccagcgggacggcatccgaggtgggctaggctcgggcccgtggcgggtgcgggggtgggagg

SEQ ID NO: 450 - RNA, 83 nt, Homo sapiens
  source 1..83: mol_type = unassigned RNA
gtctactcccagggtgccaagctgtttcgtgttccctccctaggggatcccaggtaggggcagcagaggacctgggcctggac

SEQ ID NO: 451 - RNA, 84 nt, Homo sapiens
  source 1..84: mol_type = unassigned RNA
ccgcagccgccgcgccgggcccgggttggccgctgacccccgcggggcccccggcggccggggcgggggcgggggctgccccgg

SEQ ID NO: 452 - RNA, 72 nt, Homo sapiens
  source 1..72: mol_type = unassigned RNA
atgagcgggtgggagcagatcttattgagagttccttctcctgctcctgattgtcttcccccaccctcacag

SEQ ID NO: 453 - RNA, 91 nt, Homo sapiens
  source 1..91: mol_type = unassigned RNA
gttctagagcatggtttctcatcatttgcactactgatacttggggtcagataattgtttgtggtgggggctgttgtttgcattgtaggat

SEQ ID NO: 454 - RNA, 75 nt, Homo sapiens
  source 1..75: mol_type = unassigned RNA
aggctggcgtgggctgagggcaggaggcctgtggccggtcccaggcctcctgcttcctgggctcaggctcggttt

SEQ ID NO: 455 - RNA, 87 nt, Homo sapiens
  source 1..87: mol_type = unassigned RNA
gtgtggccggcaggcgggtgggcgggggcggccggtgggaaccccgccccgccccgcgcccgcactcacccgcccgtctccccacag

SEQ ID NO: 456 - RNA, 76 nt, Homo sapiens
  source 1..76: mol_type = unassigned RNA
ggggtcacctctctggccgtctaccttccacactgacaagggccgtggggacgtagctggccagacaggtgacccc

SEQ ID NO: 457 - RNA, 100 nt, Homo sapiens
  source 1..100: mol_type = unassigned RNA
gaggtgggaggattgcttgagtcagggtggttgaggctgcagtaagttgtgatcataccactgcactccagcctgagtgacagagcaagaccttgtctca

SEQ ID NO: 458 - RNA, 67 nt, Homo sapiens
  source 1..67: mol_type = unassigned RNA
aatagattattggtcaccacctccagtttctgaatttgtgagactggggtggggcctgagaatttgc

SEQ ID NO: 459 - RNA, 90 nt, Homo sapiens
  source 1..90: mol_type = unassigned RNA
ggcgcctctgcagctccggctccccctggcctctcgggaactacaagtcccagggggcctggcggtgggcggcgggcggaagaggcgggg

SEQ ID NO: 460 - RNA, 64 nt, Homo sapiens
  source 1..64: mol_type = unassigned RNA
gggtgggggcggggcggcaggggcctcccccagtgccaggccccattctgcttctctcccagct

SEQ ID NO: 461 - RNA, 64 nt, Homo sapiens
  source 1..64: mol_type = unassigned RNA
agttcagggccgaagggtggaagctgctggtgctcatctcagcctctgcccttggcctccccag

SEQ ID NO: 462 - RNA, 68 nt, Homo sapiens
  source 1..68: mol_type = unassigned RNA
cgtggtgaggatatggcagggaaggggagtttccctctattcccttccccccagtaatcttcatcatg

SEQ ID NO: 463 - RNA, 67 nt, Homo sapiens
  source 1..67: mol_type = unassigned RNA
gcgccctccctctctccccggtgtgcaaatgtgtgtgtgcggtgttatgccggacaagagggaggtg

SEQ ID NO: 464 - RNA, 98 nt, Homo sapiens
  source 1..98: mol_type = unassigned RNA
cgaggtaggggcgtcccgggcgcgcgggcgggtcccaggctgggcccctcggaggccgggtgctcactgccccgtcccggcgcccgtgtctcctccag

SEQ ID NO: 465 - RNA, 69 nt, Homo sapiens
  source 1..69: mol_type = unassigned RNA
tggggtaggggtgggggaattcaggggtgtcgaactcatggctgccacctttgtgtccccatcctgcag

SEQ ID NO: 466 - RNA, 68 nt, Homo sapiens
  source 1..68: mol_type = unassigned RNA
gcatcctgtactgagctgccccgaggcccttcatgctgcccagctcggggcagctcagtacaggatac

SEQ ID NO: 467 - RNA, 64 nt, Homo sapiens
  source 1..64: mol_type = unassigned RNA
tcctgtactgagctgccccgagctgggcagcatgaagggcctcggggcagctcagtacaggatg

SEQ ID NO: 468 - RNA, 110 nt, Homo sapiens
  source 1..110: mol_type = unassigned RNA
gtctaccaggtgtgggcccagctttacatagttcatgctgaggccgggatttcatgcagaaaactggttgcaaaaggtgctgaaggggctgggggagcacaagggagaag

SEQ ID NO: 469 - RNA, 71 nt, Homo sapiens
  source 1..71: mol_type = unassigned RNA
gtgtctgtgccggtcccaggagaacctgcagaggcatcgggtcagcggtgctcctgcgggccgacactcac

SEQ ID NO: 470 - RNA, 99 nt, Homo sapiens
  source 1..99: mol_type = unassigned RNA
cgcccacctcagcctcccaaaatgctgggattacaggcatgagccactgcggtcgaccatgacctggacatgtttgtgcccagtactgtcagtttgcag

SEQ ID NO: 471 - RNA, 86 nt, Homo sapiens
  source 1..86: mol_type = unassigned RNA
agcactgcccccggtgagtcagggtggggctggccccctgcttcgtgcccatccgcgctctgactctctgcccacctgcaggagct

SEQ ID NO: 472 - RNA, 65 nt, Homo sapiens
  source 1..65: mol_type = unassigned RNA
ctcctctgggggtggggggctgggcgtggtggacagcgatgcatccctcgccttctcaccctcag

SEQ ID NO: 473 - RNA, 73 nt, Homo sapiens
  source 1..73: mol_type = unassigned RNA
ggggaggtacctgggacaggaggaggaggcagccttgcctcagaaaccaaactgtcaaaagtgtaggttccac

SEQ ID NO: 474 - RNA, 55 nt, Homo sapiens
  source 1..55: mol_type = unassigned RNA
ccggatccgagtcacggcaccaaatttcatgcgtgtccgtgtgaagagaccacca

SEQ ID NO: 475 - RNA, 66 nt, Homo sapiens
  source 1..66: mol_type = unassigned RNA
acccaggaggcggaggaggtggaggttgcagtgagccaagatcgtggcactgactccagcctgggg

SEQ ID NO: 476 - RNA, 94 nt, Homo sapiens
  source 1..94: mol_type = unassigned RNA
cacggtgtcccctggtggaacctggcagggggagaggtaaggtctttcagcctctccaaagcccatggtcaggtactcaggtgggggagccctg

SEQ ID NO: 477 - RNA, 62 nt, Homo sapiens
  source 1..62: mol_type = unassigned RNA
gagggtggtggaggaagagggcagctcccatgactgcctgaccgccttctctcctcccccag

SEQ ID NO: 478 - RNA, 66 nt, Homo sapiens
  source 1..66: mol_type = unassigned RNA
agcagccctcggcggcccggggggcgggcggcggtgcccgtcccggggctgcgcgaggcacaggcg

SEQ ID NO: 479 - RNA, 81 nt, Homo sapiens
  source 1..81: mol_type = unassigned RNA
tagaggcagtttcaacagatgtgtagacttttgatatgagaaattggtttcaaaatcaggagtcggggctttactgctttt

SEQ ID NO: 480 - RNA, 74 nt, Homo sapiens
  source 1..74: mol_type = unassigned RNA
cctgcaggaggcagtgggcgagcaggcggggcagcccaatgccatgggcctgatctcaccgctgcctccttccc

SEQ ID NO: 481 - RNA, 109 nt, Homo sapiens
  source 1..109: mol_type = unassigned RNA
tgctattgtcttactgctacagcagggctggggattgcagtatccgctgttgctgctgctcccagtcctgcccctgctgctacctagtccagcctcaccgcatcccaga

SEQ ID NO: 482 - RNA, 67 nt, Homo sapiens
  source 1..67: mol_type = unassigned RNA
cagcgtgggctgctgagaaggggcagggtcctccagctcattcctcctgcctcctccgtggcctcag

SEQ ID NO: 483 - RNA, 63 nt, Homo sapiens
  source 1..63: mol_type = unassigned RNA
gcatgctgggcgaggctggcatctagcacaggcggtagatgcttgctcttgccattgcaatga

SEQ ID NO: 484 - RNA, 98 nt, Homo sapiens
  source 1..98: mol_type = unassigned RNA
cttggtcaataggaaagaggtgggacctcctggcttttcctctgcagcatggctcggacctagtgcaatgtttaagctcccctctctttcctgttcag

SEQ ID NO: 485 - RNA, 70 nt, Homo sapiens
  source 1..70: mol_type = unassigned RNA
aggacccagcggggctgggcgcgcggagcagcgctgggtgcagcgcctgcgccggcagctgcaagggccg

SEQ ID NO: 486 - RNA, 61 nt, Homo sapiens
  source 1..61: mol_type = unassigned RNA
tccgctctgtggagtggggtgcctgtcccctgccactgggtgacccacccctctccaccag

SEQ ID NO: 487 - RNA, 83 nt, Homo sapiens
  source 1..83: mol_type = unassigned RNA
aatagagggtgcacaggcacgggagctcaggtgaggcagggagctgagctcacctgacctcccatgcctgtgcaccctctatt

SEQ ID NO: 488 - RNA, 87 nt, Homo sapiens
  source 1..87: mol_type = unassigned RNA
gtgaggcggggccaggagggtgtgtggcgtgggtgctgcggggccgtcagggtgcctgcgggacgctcacctggctggcccgcccag

SEQ ID NO: 489 - RNA, 99 nt, Homo sapiens
  source 1..99: mol_type = unassigned RNA
gcaagggacagggagggtcgtggcgacactcgcgccagctcccgggacggctgggctcgggctggtcgccgacctccgaccctccactagatgcctggc

SEQ ID NO: 490 - RNA, 91 nt, Homo sapiens
  source 1..91: mol_type = unassigned RNA
ggcgccccggctccccgcgcccccgatcggggccgccgctagtagtggcggcggcggaggcgggggcagcggcggcggcggcggaggcgcc

SEQ ID NO: 491 - RNA, 69 nt, Homo sapiens
  source 1..69: mol_type = unassigned RNA
ggccctcgggcctggggttgggggagctctgtcctgtctcactcattgctcctcccctgcctggcccag

SEQ ID NO: 492 - RNA, 89 nt, Homo sapiens
  source 1..89: mol_type = unassigned RNA
tctccgtttatcccaccactgccaccattattgctactgttcagcaggtgctgctggtggtgatggtgatagtctggtgggggcggtgg

SEQ ID NO: 493 - RNA, 69 nt, Homo sapiens
  source 1..69: mol_type = unassigned RNA
gaggaggggaggtgtgcagggctggggtcactgactctgcttcccctgccctgcatggtgtccccacag

SEQ ID NO: 494 - RNA, 81 nt, Homo sapiens
  source 1..81: mol_type = unassigned RNA
agttggtgggggagccatgagataagagcacctcctagagaatgttgaactaaaggtgccctctctggctcctccccaaag

SEQ ID NO: 495 - RNA, 67 nt, Homo sapiens
  source 1..67: mol_type = unassigned RNA
tacaggccggggctttgggtgagggacccccggagtctgtcacggtctcaccccaactctgccccag

SEQ ID NO: 496 - RNA, 119 nt, Homo sapiens
  source 1..119: mol_type = unassigned RNA
ccatgaggagctggcagtgggatggcctgggggtaggagcgtggcttctggagctagaccacatgggttcagatcccagcggtgcctctaactggccacaggaccttgggcagtcagct

SEQ ID NO: 497 - RNA, 62 nt, Homo sapiens
  source 1..62: mol_type = unassigned RNA
cttcctggtgggtggggaggagaagtgccgtcctcatgagcccctctctgtcccacccatag

SEQ ID NO: 498 - RNA, 87 nt, Homo sapiens
  source 1..87: mol_type = unassigned RNA
gcagggctggcagggaggctgggaggggctggctgggtctggtagtgggcatcagctggccctcatttcttaagacagcacttctgt

SEQ ID NO: 499 - RNA, 97 nt, Homo sapiens
  source 1..97: mol_type = unassigned RNA
atctgagttgggagggtccctctccaaatgtgtcttggggtgggggatcaagacacatttggagagggaacctcccaactcggcctctgccatcatt

SEQ ID NO: 500 - RNA, 73 nt, Homo sapiens
  source 1..73: mol_type = unassigned RNA
ggaggctgggctgggacggacacccggcctccactttctgtggcaggtacctcctccatgtcggcccgccttg

SEQ ID NO: 501 - RNA, 70 nt, Homo sapiens
  source 1..70: mol_type = unassigned RNA
acaaatagcttcagggagtcaggggagggcagaaatagatggccttcccctgctgggaagaaagtgggtc

SEQ ID NO: 502 - RNA, 87 nt, Homo sapiens
  source 1..87: mol_type = unassigned RNA
ccctcatctctgggcaggggcttattgtaggagtctctgaagagagctgtggactgacctgctttaacccttccccaggttcccatt

SEQ ID NO: 503 - RNA, 72 nt, Homo sapiens
  source 1..72: mol_type = unassigned RNA
ctgggggaggaaggacaggccatctgctattcgtccaccaacctgacttgatcctctcttccctcctcccag

SEQ ID NO: 504 - RNA, 67 nt, Homo sapiens
  source 1..67: mol_type = unassigned RNA
ccagacccctggggctgggcaggcggaaagaggtctgaactgcctctgcctccttggtctccggcag

SEQ ID NO: 505 - RNA, 65 nt, Homo sapiens
  source 1..65: mol_type = unassigned RNA
gtctcctggggggaggagaccctgctctccctggcagcaagcctctcctgcccttccagattagc

SEQ ID NO: 506 - RNA, 91 nt, Homo sapiens
  source 1..91: mol_type = unassigned RNA
acgccccccgccccgccaccgccttggaggctgacctcttactttcggtcggtcttcttccctgggcttggtttgggggcgggggagtgtc

SEQ ID NO: 507 - RNA, 57 nt, Homo sapiens
  source 1..57: mol_type = unassigned RNA
cattggagggtgtggaagacatctgggccaactctgatctcttcatctaccccccag

SEQ ID NO: 508 - RNA, 67 nt, Homo sapiens
  source 1..67: mol_type = unassigned RNA
cagggaggaggtggtactaggggccagcaacctgattacccctctttggccctttgtacccctccag

SEQ ID NO: 509 - RNA, 96 nt, Homo sapiens
  source 1..96: mol_type = unassigned RNA
gctctggggcgtgccgccgccgtcgctgccacctcccctaccgctagtggaagaagatggcggaaggcggagcggcggatctggacacccagcggt

SEQ ID NO: 510 - RNA, 61 nt, Homo sapiens
  source 1..61: mol_type = unassigned RNA
cctgcggggacaggccagggcatctaggctgtgcacagtgacgcccctcctgcccccacag

SEQ ID NO: 511 - RNA, 58 nt, Homo sapiens
  source 1..58: mol_type = unassigned RNA
agggccagaggagcctggagtggtcgggtcgactgaacccaggttccctctggccgca

SEQ ID NO: 512 - RNA, 86 nt, Homo sapiens
  source 1..86: mol_type = unassigned RNA
attctaggtggggagactgacggctggaggcccataagctgtctaaaacttcggcccccagatttctggtctccccacttcagaac

SEQ ID NO: 513 - RNA, 80 nt, Homo sapiens
  source 1..80: mol_type = unassigned RNA
ggcgcgtcgcccccctcagtccaccagagcccggatacctcagaaattcggctctgggtctgtggggagcgaaatgcaac

SEQ ID NO: 514 - RNA, 98 nt, Homo sapiens
  source 1..98: mol_type = unassigned RNA
ttctcacccccgcctgacacgggcgacagctgcggcccgctgtgttcactcgggccgagtgcgtctcctgtcaggcaagggagagcagagcccccctg

SEQ ID NO: 515 - RNA, 94 nt, Homo sapiens
  source 1..94: mol_type = unassigned RNA
ataaaggaagttaggctgaggggcagagagcgagacttttctattttccaaaagctcggtctgaggcccctcagtcttgcttcctaacccgcgc

SEQ ID NO: 516 - RNA, 75 nt, Homo sapiens
  source 1..75: mol_type = unassigned RNA
tcatccctgggtggggatttgttgcattacttgtgttctatataaagtattgcacttgtcccggcctgtggaaga

SEQ ID NO: 517 - RNA, 89 nt, Homo sapiens
  source 1..89: mol_type = unassigned RNA
gtcagcagtgccttagcagcacgtaaatattggcgttaagattctaaaattatctccagtattaactgtgctgctgaagtaaggttgac

SEQ ID NO: 518 - RNA, 81 nt, Homo sapiens
  source 1..81: mol_type = unassigned RNA
gttccactctagcagcacgtaaatattggcgtagtgaaatatatattaaacaccaatattactgtgctgctttagtgtgac

SEQ ID NO: 519 - RNA, 72 nt, Homo sapiens
  source 1..72: mol_type = unassigned RNA
cttgggaatggcaaggaaaccgttaccattactgagtttagtaatggtaatggttctcttgctatacccaga

SEQ ID NO: 520 - RNA, 90 nt, Homo sapiens
  source 1..90: mol_type = unassigned RNA
aggcctcgctgttctctatggctttttattcctatgtgattctactgctcactcatatagggattggagccgtggcgcacggcggggaca

SEQ ID NO: 521 - RNA, 86 nt, Homo sapiens
  source 1..86: mol_type = unassigned RNA
ggcttagaaacagtccctaggtaggatttggggaggagctaagaagcccctacagggcccagaggtggggactgagccttagttgg

SEQ ID NO: 522 - RNA, 96 nt, Homo sapiens
  source 1..96: mol_type = unassigned RNA
cgggccccgggcgggcgggagggacgggacgcggtgcagtgttgttttttcccccgccaatattgcactcgtcccggcctccggcccccccggccc

SEQ ID NO: 523 - RNA, 70 nt, Homo sapiens
  source 1..70: mol_type = unassigned RNA
ggccgcggcgcgcaagatggcggcgggcccgggcaccgccccttccgccccgccgggcgtcgcacgaggc

SEQ ID NO: 524 - RNA, 94 nt, Homo sapiens
  source 1..94: mol_type = unassigned RNA
gtgaggtgtgggcccggccccaggagcggggcctgggcagccccgtgtgttgaggaaggaaggcagggcccccgctccccgggcctgaccccac

SEQ ID NO: 525 - RNA, 97 nt, Homo sapiens
  source 1..97: mol_type = unassigned RNA
ctcaggtgctctggctgcttgggttcctggcatgctgatttgtgacttaagattaaaatcacattgccagggattaccacgcaaccacgaccttggc

SEQ ID NO: 526 - RNA, 78 nt, Homo sapiens
  source 1..78: mol_type = unassigned RNA
ctttctacacaggttgggatcggttgcaatgctgtgtttctgtatggtattgcacttgtcccggcctgttgagtttgg

SEQ ID NO: 527 - RNA, 75 nt, Homo sapiens; identical to SEQ ID NO: 516
  source 1..75: mol_type = unassigned RNA

SEQ ID NO: 528 - RNA, 100 nt, Homo sapiens
  source 1..100: mol_type = unassigned RNA
gctcggttgccgtggttgcgggccctgcccgcccgccagctcgctgacagcacgactcagggcggagggaagtaggtccgttggtcggtcgggaacgagg

SEQ ID NO: 529 - RNA, 65 nt, Homo sapiens
  source 1..65: mol_type = unassigned RNA
agccctgggggtggtctctagccaaggctctggggtctcacccttggctggtctctgctccgcag

SEQ ID NO: 530 - RNA, 85 nt, Homo sapiens
  source 1..85: mol_type = unassigned RNA
ggggaggtagggaaaaggaagggggaggagaaggtgagaccaatgtcctgggtgccactcctgcccagtgcctcccttcctcgtt

SEQ ID NO: 531 - RNA, 87 nt, Homo sapiens; identical to SEQ ID NO: 488
  source 1..87: mol_type = unassigned RNA

SEQ ID NO: 532 - RNA, 65 nt, Homo sapiens
  source 1..65: mol_type = unassigned RNA
gcttctgggaggaggggatcttgggagtgatcccaacagctgagctccctgaatccctgtcccag

SEQ ID NO: 533 - RNA, 60 nt, Homo sapiens
  source 1..60: mol_type = unassigned RNA
tgtgaatgacccccttccagagccaaaatcaccagggatggaggaggggtcttgggtact

SEQ ID NO: 534 - RNA, 68 nt, Homo sapiens
  source 1..68: mol_type = unassigned RNA
tgcccaggctggagcgagtgcagtggtgcagtcagtcctagctcactgcagcctcgaactcctgggct

SEQ ID NO: 535 - RNA, 80 nt, Homo sapiens
  source 1..80: mol_type = unassigned RNA
acctgaggagccagccctcctcccgcacccaaacttggagcacttgacctttggctgttggagggggcaggctcgcgggt

SEQ ID NO: 536 - RNA, 83 nt, Homo sapiens
  source 1..83: mol_type = unassigned RNA
ggcccggctccgggtctcggcccgtacagtccggccggccatgctggcggggctggggccggggccgagcccgcggcggggcc

SEQ ID NO: 537 - RNA, 83 nt, Homo sapiens
  source 1..83: mol_type = unassigned RNA
acggcatctttgcactcagcaggcaggctggtgcagcccgtggtgggggaccatcctgcctgctgtggggtaaggacggctgt

SEQ ID NO: 538 - RNA, 63 nt, Homo sapiens
  source 1..63: mol_type = unassigned RNA
tggtggcggcggtagttatgggcttctctttctcaccagcagcccctgggccgccgcctccct

SEQ ID NO: 539 - RNA, 98 nt, Homo sapiens
  source 1..98: mol_type = unassigned RNA
agaatgggcaaatgaacagtaaatttggaggcctggggccctccctgctgctggagaagtgtttgcacgggtgggccttgtctttgaaaggaggtgga

SEQ ID NO: 540 - RNA, 102 nt, Homo sapiens
  source 1..102: mol_type = unassigned RNA
gtgggagggcccaggcgcgggcaggggtgggggtggcagagcgctgtcccgggggcggggccgaagcgcggcgaccgtaactccttctgctccgtcccccag

SEQ ID NO: 541 - RNA, 80 nt, Homo sapiens; identical to SEQ ID NO: 317
  source 1..80: mol_type = unassigned RNA

SEQ ID NO: 542 - RNA, 93 nt, Homo sapiens
  source 1..93: mol_type = unassigned RNA
tacttatggcaccccactcctggtaccatagtcataagttaggagatgttagagctgtgagtaccatgacttaagtgtggtggcttaaacatg

SEQ ID NO: 543 - RNA, 66 nt, Homo sapiens
  source 1..66: mol_type = unassigned RNA
cctggaggggggcactgcgcaagcaaagccagggaccctgagaggctttgcttcctgctcccctag

SEQ ID NO: 544 - RNA, 65 nt, Homo sapiens
  source 1..65: mol_type = unassigned RNA
ttctcctggggagtggctggggagcagacagacccaacctcatgctccccggcctctgcccccag

SEQ ID NO: 545 - RNA, 22 nt, Homo sapiens
  source 1..22: mol_type = unassigned RNA
ccccggtgttggggcgcgtctg

SEQ ID NO: 546 - RNA, 21 nt, Homo sapiens
  source 1..21: mol_type = unassigned RNA
cccggtgttggggcgcgtctg

SEQ ID NO: 547 - RNA, 22 nt, Homo sapiens
  source 1..22: mol_type = unassigned RNA
tggcggagcccattccatgcca

SEQ ID NO: 548 - RNA, 21 nt, Homo sapiens
  source 1..21: mol_type = unassigned RNA
ctggcggagcccattccatgc

SEQ ID NO: 549 - RNA, 25 nt, Homo sapiens
  source 1..25: mol_type = unassigned RNA
actcggcgtggcgtcggtcgtggta

SEQ ID NO: 550 - RNA, 15 nt, Homo sapiens
  source 1..15: mol_type = unassigned RNA
actcggcgtggcgtc

SEQ ID NO: 551 - RNA, 19 nt, Homo sapiens
  source 1..19: mol_type = unassigned RNA
cggcgcgaccggcccgggg

SEQ ID NO: 552 - RNA, 19 nt, Homo sapiens
  source 1..19: mol_type = unassigned RNA
cggcgcgaccggcccgggg

SEQ ID NO: 553 - RNA, 26 nt, Homo sapiens
  source 1..26: mol_type = unassigned RNA
cggtgggatcccgcggccgtgttttc

SEQ ID NO: 554 - RNA, 15 nt, Homo sapiens
  source 1..15: mol_type = unassigned RNA
ggggcgccgcgggac

SEQ ID NO: 555 - RNA, 20 nt, Homo sapiens
  source 1..20: mol_type = unassigned RNA
ccccggggagcccggcggtg

SEQ ID NO: 556 - RNA, 15 nt, Homo sapiens
  source 1..15: mol_type = unassigned RNA
accccggggagcccg

SEQ ID NO: 557 - RNA, 24 nt, Homo sapiens
  source 1..24: mol_type = unassigned RNA
gatcggtcgagagcgtcctggctg

SEQ ID NO: 558 - RNA, 15 nt, Homo sapiens
  source 1..15: mol_type = unassigned RNA
gctgggcggggcgcg

SEQ ID NO: 559 - RNA, 23 nt, Homo sapiens
  source 1..23: mol_type = unassigned RNA
tgtagagcagggagcaggaagct

SEQ ID NO: 560 - RNA, 15 nt, Homo sapiens
  source 1..15: mol_type = unassigned RNA
cagggagcaggaagc

SEQ ID NO: 561 - RNA, 15 nt, Homo sapiens
  source 1..15: mol_type = unassigned RNA
tgaagcgggggggcg

SEQ ID NO: 562 - RNA, 15 nt, Homo sapiens
  source 1..15: mol_type = unassigned RNA
tgaagcgggggggcg

SEQ ID NO: 563 - RNA, 22 nt, Homo sapiens
  source 1..22: mol_type = unassigned RNA
ctgggctcgggacgcgcggctc

SEQ ID NO: 564 - RNA, 19 nt, Homo sapiens
  source 1..19: mol_type = unassigned RNA
ctgggctcgggacgcgcgg

SEQ ID NO: 565 - RNA, 23 nt, Homo sapiens
  source 1..23: mol_type = unassigned RNA
cacaggtgaggttcttgggagcc

SEQ ID NO: 566 - RNA, 15 nt, Homo sapiens
  source 1..15: mol_type = unassigned RNA
acaggtgaggttctt

SEQ ID NO: 567 - RNA, 19 nt, Homo sapiens
  source 1..19: mol_type = unassigned RNA
agtgggaggccagggcacg

SEQ ID NO: 568 - RNA, 15 nt, Homo sapiens
  source 1..15: mol_type = unassigned RNA
agggggagctgcagg

SEQ ID NO: 569 - RNA, 22 nt, Homo sapiens
  source 1..22: mol_type = unassigned RNA
aggcagcggggtgtagtggata

SEQ ID NO: 570 - RNA, 21 nt, Homo sapiens
  source 1..21: mol_type = unassigned RNA
aggcagcggggtgtagtggat

SEQ ID NO: 571 - RNA, 22 nt, Homo sapiens
  source 1..22: mol_type = unassigned RNA
ggggggcaggaggggctcaggg

SEQ ID NO: 572 - RNA, 16 nt, Homo sapiens
  source 1..16: mol_type = unassigned RNA
gtgggggggcaggagg

SEQ ID NO: 573 - RNA, 21 nt, Homo sapiens
  source 1..21: mol_type = unassigned RNA
gaatggatttttggagcagga

SEQ ID NO: 574 - RNA, 15 nt, Homo sapiens
  source 1..15: mol_type = unassigned RNA
gaatggatttttgga

SEQ ID NO: 575 - RNA, 24 nt, Homo sapiens
  source 1..24: mol_type = unassigned RNA
gctgcgggctgcggtcagggcgat

SEQ ID NO: 576 - RNA, 20 nt, Homo sapiens
  source 1..20: mol_type = unassigned RNA
gctgcgggctgcggtcaggg

SEQ ID NO: 577 - RNA, 18 nt, Homo sapiens
  source 1..18: mol_type = unassigned RNA
gcggcggcggcggcagca

SEQ ID NO: 578 - RNA, 15 nt, Homo sapiens
  source 1..15: mol_type = unassigned RNA
gcgggcggcggcggc

SEQ ID NO: 579 - RNA, 22 nt, Homo sapiens
  source 1..22: mol_type = unassigned RNA
tgggaggggagaggcagcaagc

SEQ ID NO: 580 - RNA, 22 nt, Homo sapiens
  source 1..22: mol_type = unassigned RNA
tgggaggggagaggcagcaagc

SEQ ID NO: 581 - RNA, 26 nt, Homo sapiens
  source 1..26: mol_type = unassigned RNA
cgggtagagagggcagtgggaggtaa

SEQ ID NO: 582 - RNA, 15 nt, Homo sapiens
  source 1..15: mol_type = unassigned RNA
cgggtagagagggca

SEQ ID NO: 583 - RNA, 23 nt, Homo sapiens
  source 1..23: mol_type = unassigned RNA
gagggagggacgggggctgtgct

SEQ ID NO: 584 - RNA, 15 nt, Homo sapiens
  source 1..15: mol_type = unassigned RNA
gaggagggagggagg

SEQ ID NO: 585 - RNA, 25 nt, Homo sapiens
  source 1..25: mol_type = unassigned RNA
caggaaggatttagggacaggcttt

SEQ ID NO: 586 - RNA, 19 nt, Homo sapiens
  source 1..19: mol_type = unassigned RNA
caggaaggatttagggaca

SEQ ID NO: 587 - RNA, 18 nt, Homo sapiens
  source 1..18: mol_type = unassigned RNA
ctccgggcggcgccgtgt

SEQ ID NO: 588 - RNA, 18 nt, Homo sapiens
  source 1..18: mol_type = unassigned RNA
ctccgggcggcgccgtgt

SEQ ID NO: 589 - RNA, 24 nt, Homo sapiens
  source 1..24: mol_type = unassigned RNA
aggcaggggctggtgctgggcggg

SEQ ID NO: 590 - RNA, 15 nt, Homo sapiens
  source 1..15: mol_type = unassigned RNA
gggcggggggcggcg

SEQ ID NO: 591 - RNA, 21 nt, Homo sapiens
  source 1..21: mol_type = unassigned RNA
ccggccgccggctccgccccg

SEQ ID NO: 592 - RNA, 17 nt, Homo sapiens
  source 1..17: mol_type = unassigned RNA
ccggccgccggctccgc

SEQ ID NO: 593 - RNA, 24 nt, Homo sapiens
  source 1..24: mol_type = unassigned RNA
ctggtacaggcctgggggacaggg

SEQ ID NO: 594 - RNA, 18 nt, Homo sapiens
  source 1..18: mol_type = unassigned RNA
ctggtacaggcctggggg

SEQ ID NO: 595 - RNA, 27 nt, Homo sapiens
  source 1..27: mol_type = unassigned RNA
gtgaacgggcgccatcccgaggctttg

SEQ ID NO: 596 - RNA, 16 nt, Homo sapiens
  source 1..16: mol_type = unassigned RNA
gtgaacgggcgccatc

SEQ ID NO: 597 - RNA, 19 nt, Homo sapiens
  source 1..19: mol_type = unassigned RNA
gtgggttggggcgggctct

SEQ ID NO: 598 - RNA, 19 nt, Homo sapiens
  source 1..19: mol_type = unassigned RNA
gtgggttggggcgggctct

SEQ ID NO: 599 - RNA, 26 nt, Homo sapiens
  source 1..26: mol_type = unassigned RNA
gcgggctgtccggaggggtcggcttt

SEQ ID NO: 600 - RNA, 16 nt, Homo sapiens
  source 1..16: mol_type = unassigned RNA
gctgtccggaggggtc

SEQ ID NO: 601 - RNA, 24 nt, Homo sapiens
  source 1..24: mol_type = unassigned RNA
cccggggcagattggtgtagggtg

SEQ ID NO: 602 - RNA, 17 nt, Homo sapiens
  source 1..17: mol_type = unassigned RNA
cggggcagattggtgta

SEQ ID NO: 603 - RNA, 23 nt, Homo sapiens
  source 1..23: mol_type = unassigned RNA
gggggtccccggtgctcggatct

SEQ ID NO: 604 - RNA, 16 nt, Homo sapiens
  source 1..16: mol_type = unassigned RNA
tcgggaggggcgggag

SEQ ID NO: 605 - RNA, 23 nt, Homo sapiens
  source 1..23: mol_type = unassigned RNA
cggggccgtagcactgtctgaga

SEQ ID NO: 606 - RNA, 20 nt, Homo sapiens
  source 1..20: mol_type = unassigned RNA
cggggccgtagcactgtctg

SEQ ID NO: 607 - RNA, 27 nt, Homo sapiens
  source 1..27: mol_type = unassigned RNA
tgagggacttttgggggcagatgtgtt

SEQ ID NO: 608 - RNA, 17 nt, Homo sapiens
  source 1..17: mol_type = unassigned RNA
ggacttttgggggcaga

SEQ ID NO: 609 - RNA, 24 nt, Homo sapiens
  source 1..24: mol_type = unassigned RNA
gaggctgggaaggcaaagggacgt

SEQ ID NO: 610 - RNA, 15 nt, Homo sapiens
  source 1..15: mol_type = unassigned RNA
gaaggaggctgggaa

SEQ ID NO: 611 - RNA, 17 nt, Homo sapiens
  source 1..17: mol_type = unassigned RNA
aggggctgggcgcgcgc

SEQ ID NO: 612 - RNA, 15 nt, Homo sapiens
  source 1..15: mol_type = unassigned RNA
caggggctgggcgcg

SEQ ID NO: 613 - RNA, 25 nt, Homo sapiens
  source 1..25: mol_type = unassigned RNA
tgcaggcagaagtggggctgacagg

SEQ ID NO: 614 - RNA, 20 nt, Homo sapiens
  source 1..20: mol_type = unassigned RNA
ctgcaggcagaagtggggct

SEQ ID NO: 615 - RNA, 23 nt, Homo sapiens
  source 1..23: mol_type = unassigned RNA
ggggggatgtgcatgctggttgg

SEQ ID NO: 616 - RNA, 15 nt, Homo sapiens
  source 1..15: mol_type = unassigned RNA
atcagcgtgcacttc

SEQ ID NO: 617 - RNA, 23 nt, Homo sapiens
  source 1..23: mol_type = unassigned RNA
caccttgccttgctgcccgggcc

SEQ ID NO: 618 - RNA, 22 nt, Homo sapiens
  source 1..22: mol_type = unassigned RNA
caccttgccttgctgcccgggc

SEQ ID NO: 619 - RNA, 23 nt, Homo sapiens
  source 1..23: mol_type = unassigned RNA
tggggcggagcttccggaggccc

SEQ ID NO: 620 - RNA, 17 nt, Homo sapiens
  source 1..17: mol_type = unassigned RNA
tggggcggagcttccgg

SEQ ID NO: 621 - RNA, 16 nt, Homo sapiens
  source 1..16: mol_type = unassigned RNA
ggacccagggagagac

SEQ ID NO: 622 - RNA, 16 nt, Homo sapiens
  source 1..16: mol_type = unassigned RNA
ggacccagggagagac

SEQ ID NO: 623 - RNA, 23 nt, Homo sapiens
  source 1..23: mol_type = unassigned RNA
ggcccggccgtgcctgaggtttc

SEQ ID NO: 624 - RNA, 15 nt, Homo sapiens
  source 1..15: mol_type = unassigned RNA
ggcggtgggatcccg

SEQ ID NO: 625 - RNA, 23 nt, Homo sapiens
  source 1..23: mol_type = unassigned RNA
cagcccgccccagccgaggttct

SEQ ID NO: 626 - RNA, 17 nt, Homo sapiens
  source 1..17: mol_type = unassigned RNA
agcccgccccagccgag

SEQ ID NO: 627 - RNA, 22 nt, Homo sapiens
  source 1..22: mol_type = unassigned RNA
actcggctgcggtggacaagtc

SEQ ID NO: 628 - RNA, 20 nt, Homo sapiens
  source 1..20: mol_type = unassigned RNA
actcggctgcggtggacaag

SEQ ID NO: 629 - RNA, 23 nt, Homo sapiens
  source 1..23: mol_type = unassigned RNA
ggtgggtgaggtcgggccccaag

SEQ ID NO: 630 - RNA, 20 nt, Homo sapiens
  source 1..20: mol_type = unassigned RNA
cggggtgggtgaggtcgggc

SEQ ID NO: 631 - RNA, 26 nt, Homo sapiens
  source 1..26: mol_type = unassigned RNA
gaggggctctcgcttctggcgccaag

SEQ ID NO: 632 - RNA, 15 nt, Homo sapiens
  source 1..15: mol_type = unassigned RNA
ggtgaggcggggggg

SEQ ID NO: 633 - RNA, 23 nt, Homo sapiens
  source 1..23: mol_type = unassigned RNA
ctgggggacgcgtgagcgcgagc

SEQ ID NO: 634 - RNA, 21 nt, Homo sapiens
  source 1..21: mol_type = unassigned RNA
ctgggggacgcgtgagcgcga

SEQ ID NO: 635 - RNA, 24 nt, Homo sapiens
  source 1..24: mol_type = unassigned RNA
gtgagtgggagccggtggggctgg

SEQ ID NO: 636 - RNA, 15 nt, Homo sapiens
  source 1..15: mol_type = unassigned RNA
ggggctggagtaagg

SEQ ID NO: 637 - RNA, 23 nt, Homo sapiens
  source 1..23: mol_type = unassigned RNA
gtgggctgggctgggctgggcca

SEQ ID NO: 638 - RNA, 15 nt, Homo sapiens
  source 1..15: mol_type = unassigned RNA
gggctgggctgggct

SEQ ID NO: 639 - RNA, 23 nt, Homo sapiens
  source 1..23: mol_type = unassigned RNA
tgcgcaggggccgggtgctcacc

SEQ ID NO: 640 - RNA, 19 nt, Homo sapiens
  source 1..19: mol_type = unassigned RNA
cgcaggggccgggtgctca

SEQ ID NO: 641 - RNA, 25 nt, Homo sapiens
  source 1..25: mol_type = unassigned RNA
ttctgggcccgcggcgggcgtgggg

SEQ ID NO: 642 - RNA, 15 nt, Homo sapiens
  source 1..15: mol_type = unassigned RNA
cgcggcgggcgtggg

SEQ ID NO: 643 - RNA, 23 nt, Homo sapiens
  source 1..23: mol_type = unassigned RNA
ctcctggggcccgcactctcgct

SEQ ID NO: 644 - RNA, 18 nt, Homo sapiens
  source 1..18: mol_type = unassigned RNA
ctcctggggcccgcactc

SEQ ID NO: 645 - RNA, 22 nt, Homo sapiens
  source 1..22: mol_type = unassigned RNA
gaaaaaggcgggagaagcccca

SEQ ID NO: 646 - RNA, 15 nt, Homo sapiens
  source 1..15: mol_type = unassigned RNA
gaaaaaggcgggaga

SEQ ID NO: 647 - RNA, 19 nt, Homo sapiens
  source 1..19: mol_type = unassigned RNA
gcaggcgaggctgggctga

SEQ ID NO: 648 - RNA, 16 nt, Homo sapiens
  source 1..16: mol_type = unassigned RNA
aggcgaggctgggctg

SEQ ID NO: 649 - RNA, 22 nt, Homo sapiens
  source 1..22: mol_type = unassigned RNA
aaaagctgggctgagaggcgac

SEQ ID NO: 650 - RNA, 15 nt, Homo sapiens
  source 1..15: mol_type = unassigned RNA
aaagctgggctgaga

SEQ ID NO: 651 - RNA, 23 nt, Homo sapiens
  source 1..23: mol_type = unassigned RNA
tggggaaggcgtcagtgtcgggt

SEQ ID NO: 652 - RNA, 16 nt, Homo sapiens
  source 1..16: mol_type = unassigned RNA
tggggaaggcgtcagt

SEQ ID NO: 653 - RNA, 23 nt, Homo sapiens
  source 1..23: mol_type = unassigned RNA
ggaggcgcaggctcggaaaggcg

SEQ ID NO: 654 - RNA, 15 nt, Homo sapiens
  source 1..15: mol_type = unassigned RNA
gcaggctcggaaagg

SEQ ID NO: 655 - RNA, 23 nt, Homo sapiens
  source 1..23: mol_type = unassigned RNA
agaagaaggcggtcggtctgcgg

SEQ ID NO: 656 - RNA, 21 nt, Homo sapiens
  source 1..21: mol_type = unassigned RNA
aagaaggcggtcggtctgcgg

SEQ ID NO: 657 - RNA, 25 nt, Homo sapiens
  source 1..25: mol_type = unassigned RNA
agcggggaggaagtgggcgctgctt

SEQ ID NO: 658 - RNA, 21 nt, Homo sapiens
  source 1..21: mol_type = unassigned RNA
agcggggaggaagtgggcgct

SEQ ID NO: 659 - RNA, 17 nt, Homo sapiens
  source 1..17: mol_type = unassigned RNA
cctccgggacggctggg

SEQ ID NO: 660 - RNA, 15 nt, Homo sapiens
  source 1..15: mol_type = unassigned RNA
ctccgggacggctgg

SEQ ID NO: 661 - RNA, 24 nt, Homo sapiens
  source 1..24: mol_type = unassigned RNA
tcgaggactggtggaagggccttt

SEQ ID NO: 662 - RNA, 16 nt, Homo sapiens
  source 1..16: mol_type = unassigned RNA
tcgaggactggtggaa

SEQ ID NO: 663 - RNA, 23 nt, Homo sapiens
  source 1..23: mol_type = unassigned RNA
ggctacaacacaggacccgggcg

SEQ ID NO: 664 - RNA, 21 nt, Homo sapiens
  source 1..21: mol_type = unassigned RNA
ggctacaacacaggacccggg

SEQ ID NO: 665 - RNA, 23 nt, Homo sapiens
  source 1..23: mol_type = unassigned RNA
tggggcggagcttccggaggccc

SEQ ID NO: 666 - RNA, 15 nt, Homo sapiens
  source 1..15: mol_type = unassigned RNA
gccccgggaaagcgt

SEQ ID NO: 667 - RNA, 21 nt, Homo sapiens
  source 1..21: mol_type = unassigned RNA
gaggcgatgtggggatgtaga

SEQ ID NO: 668 - RNA, 20 nt, Homo sapiens
  source 1..20: mol_type = unassigned RNA
cccagtctcatttcctcatc

SEQ ID NO: 669 - RNA, 21 nt, Homo sapiens
  source 1..21: mol_type = unassigned RNA
gagggcagcgtgggtgtggcg

SEQ ID NO: 670 - RNA, 21 nt, Homo sapiens
  source 1..21: mol_type = unassigned RNA
gagggcagcgtgggtgtggcg

SEQ ID NO: 671 - RNA, 22 nt, Homo sapiens
  source 1..22: mol_type = unassigned RNA
accaggaggctgaggcccctca

SEQ ID NO: 672 - RNA, 15 nt, Homo sapiens
  source 1..15: mol_type = unassigned RNA
accaggaggctgagg

SEQ ID NO: 673 - RNA, 21 nt, Homo sapiens
  source 1..21: mol_type = unassigned RNA
ttgaggagacatggtgggggc

SEQ ID NO: 674 - RNA, 15 nt, Homo sapiens
  source 1..15: mol_type = unassigned RNA
ttgaggagacatggt

SEQ ID NO: 675 - RNA, 22 nt, Homo sapiens
  source 1..22: mol_type = unassigned RNA
ggcagggacagcaaaggggtgc

SEQ ID NO: 676 - RNA, 18 nt, Homo sapiens
  source 1..18: mol_type = unassigned RNA
gcagggacagcaaagggg

SEQ ID NO: 677 - RNA, 25 nt, Homo sapiens
  source 1..25: mol_type = unassigned RNA
ctgcaggcagaagtggggctgacag

SEQ ID NO: 678 - RNA, 19 nt, Homo sapiens
  source 1..19: mol_type = unassigned RNA
caggcagaagtggggctga

SEQ ID NO: 679 - RNA, 24 nt, Homo sapiens
  source 1..24: mol_type = unassigned RNA
cgctcgggcggaggtggttgagtg

SEQ ID NO: 680 - RNA, 17 nt, Homo sapiens
  source 1..17: mol_type = unassigned RNA
tcgggcggaggtggttg

SEQ ID NO: 681 - RNA, 16 nt, Homo sapiens
  source 1..16: mol_type = unassigned RNA
gtgaaggcccggcgga

SEQ ID NO: 682 - RNA, 15 nt, Homo sapiens
  source 1..15: mol_type = unassigned RNA
gtgaaggcccggcgg

SEQ ID NO: 683 - RNA, 17 nt, Homo sapiens
  source 1..17: mol_type = unassigned RNA
tgcaggggcaggccagc

SEQ ID NO: 684 - RNA, 17 nt, Homo sapiens
  source 1..17: mol_type = unassigned RNA
tgcaggggcaggccagc

SEQ ID NO: 685 - RNA, 25 nt, Homo sapiens
  source 1..25: mol_type = unassigned RNA
tgcggggctagggctaacagcagtc

SEQ ID NO: 686 - RNA, 15 nt, Homo sapiens
  source 1..15: mol_type = unassigned RNA
tgcggggctagggct

SEQ ID NO: 687 - RNA, 17 nt, Homo sapiens
  source 1..17: mol_type = unassigned RNA
ggtgagcgctcgctggc

SEQ ID NO: 688 - RNA, 15 nt, Homo sapiens
  source 1..15: mol_type = unassigned RNA
cggtgagcgctcgct

SEQ ID NO: 689 - RNA, 15 nt, Homo sapiens
  source 1..15: mol_type = unassigned RNA
ggcgcggggaggtgc

SEQ ID NO: 690 - RNA, 15 nt, Homo sapiens
  source 1..15: mol_type = unassigned RNA
ggcgcggggaggtgc

SEQ ID NO: 691 - RNA, 18 nt, Homo sapiens
  source 1..18: mol_type = unassigned RNA
gaggctgaaggaagatgg

SEQ ID NO: 692 - RNA, 15 nt, Homo sapiens
  source 1..15: mol_type = unassigned RNA
gaggctgaaggaaga

SEQ ID NO: 693 - RNA, 23 nt, Homo sapiens
  source 1..23: mol_type = unassigned RNA
ccagggctggcagtgacatgggt

SEQ ID NO: 694 - RNA, 19 nt, Homo sapiens
  source 1..19: mol_type = unassigned RNA
cagggctggcagtgacatg

SEQ ID NO: 695 - RNA, 16 nt, Homo sapiens
  source 1..16: mol_type = unassigned RNA
tgctggtgatgctttc

SEQ ID NO: 696 - RNA, 16 nt, Homo sapiens
  source 1..16: mol_type = unassigned RNA
tgctggtgatgctttc

SEQ ID NO: 697 - RNA, 22 nt, Homo sapiens
  source 1..22: mol_type = unassigned RNA
gagggttgggtggaggctctcc

SEQ ID NO: 698 - RNA, 15 nt, Homo sapiens
  source 1..15: mol_type = unassigned RNA
gagggttgggtggag

SEQ ID NO: 699 - RNA, 20 nt, Homo sapiens
  source 1..20: mol_type = unassigned RNA
tgagtggggctcccgggacg

SEQ ID NO: 700 - RNA, 20 nt, Homo sapiens
  source 1..20: mol_type = unassigned RNA
tgagtggggctcccgggacg

SEQ ID NO: 701 - RNA, 21 nt, Homo sapiens
  source 1..21: mol_type = unassigned RNA
cccagcaggacgggagcgcgg

SEQ ID NO: 702 - RNA, 16 nt, Homo sapiens
  source 1..16: mol_type = unassigned RNA
aagctgggtcaaggag

SEQ ID NO: 703 - RNA, 20 nt, Homo sapiens
  source 1..20: mol_type = unassigned RNA
ggctggtcagatgggagtgg

SEQ ID NO: 704 - RNA, 20 nt, Homo sapiens
  source 1..20: mol_type = unassigned RNA
ggctggtcagatgggagtgg

SEQ ID NO: 705 - RNA, 23 nt, Homo sapiens
  source 1..23: mol_type = unassigned RNA
acaggagtgggggtgggacataa

SEQ ID NO: 706 - RNA, 20 nt, Homo sapiens
  source 1..20: mol_type = unassigned RNA
acaggagtgggggtgggaca

SEQ ID NO: 707 - RNA, 22 nt, Homo sapiens
  source 1..22: mol_type = unassigned RNA
gtgggcgggggcaggtgtgtgg

SEQ ID NO: 708 - RNA, 15 nt, Homo sapiens
  source 1..15: mol_type = unassigned RNA
cgggggcaggtgtgt

SEQ ID NO: 709 - RNA, 21 nt, Homo sapiens
  source 1..21: mol_type = unassigned RNA
gccgggcgtggtggtgggggc

SEQ ID NO: 710 - RNA, 15 nt, Homo sapiens
  source 1..15: mol_type = unassigned RNA
tagccgggcgtggtg

SEQ ID NO: 711 - RNA, 29 nt, Homo sapiens
  source 1..29: mol_type = unassigned RNA
ggtcaggcggctcggactgagcaggtggg

SEQ ID NO: 712 - RNA, 15 nt, Homo sapiens
  source 1..15: mol_type = unassigned RNA
agagtgtggtcaggc

SEQ ID NO: 713 - RNA, 22 nt, Homo sapiens
  source 1..22: mol_type = unassigned RNA
cagaaggggagttgggagcaga

SEQ ID NO: 714 - RNA, 16 nt, Homo sapiens
  source 1..16: mol_type = unassigned RNA
gaaggggagttgggag

SEQ ID NO: 715 - RNA, 16 nt, Homo sapiens
  source 1..16: mol_type = unassigned RNA
ggcgcggagggcggac

SEQ ID NO: 716 - RNA, 15 nt, Homo sapiens
  source 1..15: mol_type = unassigned RNA
ggcgcggagggcgga

SEQ ID NO: 717 - RNA, 21 nt, Homo sapiens
  source 1..21: mol_type = unassigned RNA
cagcaggggagagagaggagt

SEQ ID NO: 718 - RNA, 20 nt, Homo sapiens
  source 1..20: mol_type = unassigned RNA
cagcaggggagagagaggag

SEQ ID NO: 719 - RNA, 26 nt, Homo sapiens
  source 1..26: mol_type = unassigned RNA
ccttctggagaggctttgtgcggata

SEQ ID NO: 720 - RNA, 15 nt, Homo sapiens
  source 1..15: mol_type = unassigned RNA
ccttctggagaggct

SEQ ID NO: 721 - RNA, 23 nt, Homo sapiens
  source 1..23: mol_type = unassigned RNA
gaaatcaagcgtgggtgagacct

SEQ ID NO: 722 - RNA, 20 nt, Homo sapiens
  source 1..20: mol_type = unassigned RNA
gaaatcaagcgtgggtgaga

SEQ ID NO: 723 - RNA, 26 nt, Homo sapiens
  source 1..26: mol_type = unassigned RNA
agggctggactcagcggcggagctgg

SEQ ID NO: 724 - RNA, 17 nt, Homo sapiens
  source 1..17: mol_type = unassigned RNA
gcggcggagctggctgc

SEQ ID NO: 725 - RNA, 24 nt, Homo sapiens
  source 1..24: mol_type = unassigned RNA
cgggcgtggtggtgggggtgggtg

SEQ ID NO: 726 - RNA, 15 nt, Homo sapiens
  source 1..15: mol_type = unassigned RNA
cgggcgtggtggtgg

SEQ ID NO: 727 - RNA, 22 nt, Homo sapiens
  source 1..22: mol_type = unassigned RNA
actcaaactgtgggggcacttt

SEQ ID NO: 728 - RNA, 19 nt, Homo sapiens
  source 1..19: mol_type = unassigned RNA
actcaaactgtgggggcac

SEQ ID NO: 729 - RNA, 16 nt, Homo sapiens
  source 1..16: mol_type = unassigned RNA
tgggggggaagaaaag

SEQ ID NO: 730 - RNA, 16 nt, Homo sapiens
  source 1..16: mol_type = unassigned RNA
tgggggggaagaaaag

SEQ ID NO: 731 - RNA, 21 nt, Homo sapiens
  source 1..21: mol_type = unassigned RNA
atccagttctctgagggggct

SEQ ID NO: 732 - RNA, 21 nt, Homo sapiens
  source 1..21: mol_type = unassigned RNA
atccagttctctgagggggct

SEQ ID NO: 733 - RNA, 24 nt, Homo sapiens
  source 1..24: mol_type = unassigned RNA
tggggagctgaggctctgggggtg

SEQ ID NO: 734 - RNA, 15 nt, Homo sapiens
  source 1..15: mol_type = unassigned RNA
ggccctggggagctg

SEQ ID NO: 735 - RNA, 22 nt, Homo sapiens
  source 1..22: mol_type = unassigned RNA
ccgggaacgtcgagactggagc

SEQ ID NO: 736 - RNA, 15 nt, Homo sapiens
  source 1..15: mol_type = unassigned RNA
cgggaacgtcgagac

SEQ ID NO: 737 - RNA, 19 nt, Homo sapiens
  source 1..19: mol_type = unassigned RNA
tggcgggtgcgggggtggg

SEQ ID NO: 738 - RNA, 15 nt, Homo sapiens
  source 1..15: mol_type = unassigned RNA
tggcgggtgcggggg

SEQ ID NO: 739 - RNA, 23 nt, Homo sapiens
  source 1..23: mol_type = unassigned RNA
taggggcagcagaggacctgggc

SEQ ID NO: 740 - RNA, 20 nt, Homo sapiens
  source 1..20: mol_type = unassigned RNA
taggggcagcagaggacctg

SEQ ID NO: 741 - RNA, 17 nt, Homo sapiens
  source 1..17: mol_type = unassigned RNA
ggggcgggggcgggggc

SEQ ID NO: 742 - RNA, 15 nt, Homo sapiens
  source 1..15: mol_type = unassigned RNA
cgcgccgggcccggg

SEQ ID NO: 743 - RNA, 23 nt, Homo sapiens
  source 1..23: mol_type = unassigned RNA
tggggacgtagctggccagacag

SEQ ID NO: 744 - RNA, 20 nt, Homo sapiens
  source 1..20: mol_type = unassigned RNA
tggggacgtagctggccaga

SEQ ID NO: 745 - RNA, 21 nt, Homo sapiens
  source 1..21: mol_type = unassigned RNA
cagcctgagtgacagagcaag

SEQ ID NO: 746 - RNA, 15 nt, Homo sapiens
  source 1..15: mol_type = unassigned RNA
actgcactccagcct

SEQ ID NO: 747 - RNA, 18 nt, Homo sapiens
  source 1..18: mol_type = unassigned RNA
gagactggggtggggcct

SEQ ID NO: 748 - RNA, 16 nt, Homo sapiens
  source 1..16: mol_type = unassigned RNA
agactggggtggggcc

SEQ ID NO: 749 - RNA, 16 nt, Homo sapiens
  source 1..16: mol_type = unassigned RNA
ggcggtgggcggcggg

SEQ ID NO: 750 - RNA, 15 nt, Homo sapiens
  source 1..15: mol_type = unassigned RNA
ggcctctcgggaact

SEQ ID NO: 751 - RNA, 17 nt, Homo sapiens
  source 1..17: mol_type = unassigned RNA
gcggggcggcaggggcc

SEQ ID NO: 752 - RNA, 15 nt, Homo sapiens
  source 1..15: mol_type = unassigned RNA
gggggcggggcggca

SEQ ID NO: 753 - RNA, 23 nt, Homo sapiens
  source 1..23: mol_type = unassigned RNA
tgaggatatggcagggaagggga

SEQ ID NO: 754 - RNA, 19 nt, Homo sapiens
  source 1..19: mol_type = unassigned RNA
tgaggatatggcagggaag

SEQ ID NO: 755 - RNA, 20 nt, Homo sapiens
  source 1..20: mol_type = unassigned RNA
ctccccggtgtgcaaatgtg

SEQ ID NO: 756 - RNA, 15 nt, Homo sapiens
  source 1..15: mol_type = unassigned RNA
gtgtgcggtgttatg

SEQ ID NO: 757 - RNA, 23 nt, Homo sapiens
  source 1..23: mol_type = unassigned RNA
cggggcagctcagtacaggatac

SEQ ID NO: 758 - RNA, 15 nt, Homo sapiens
  source 1..15: mol_type = unassigned RNA
agctcagtacaggat

SEQ ID NO: 759 - RNA, 24 nt, Homo sapiens
  source 1..24: mol_type = unassigned RNA
cccaaaatgctgggattacaggca

SEQ ID NO: 760 - RNA, 15 nt, Homo sapiens
  source 1..15: mol_type = unassigned RNA
gcccacctcagcctc

SEQ ID NO: 761 - RNA, 21 nt, Homo sapiens
  source 1..21: mol_type = unassigned RNA
gtgagtcagggtggggctggc

SEQ ID NO: 762 - RNA, 21 nt, Homo sapiens
  source 1..21: mol_type = unassigned RNA
gtgagtcagggtggggctggc

SEQ ID NO: 763 - RNA, 15 nt, Homo sapiens
  source 1..15: mol_type = unassigned RNA
aggaggaggaggcag

SEQ ID NO: 764 - RNA, 15 nt, Homo sapiens
  source 1..15: mol_type = unassigned RNA
aggaggaggaggcag

SEQ ID NO: 765 - RNA, 21 nt, Homo sapiens
  source 1..21: mol_type = unassigned RNA
cggatccgagtcacggcacca

SEQ ID NO: 766 - RNA, 15 nt, Homo sapiens
  source 1..15: mol_type = unassigned RNA
ggatccgagtcacgg

SEQ ID NO: 767 - RNA, 23 nt, Homo sapiens
  source 1..23: mol_type = unassigned RNA
ccaggaggcggaggaggtggagg

SEQ ID NO: 768 - RNA, 15 nt, Homo sapiens
  source 1..15: mol_type = unassigned RNA
acccaggaggcggag

SEQ ID NO: 769 - RNA, 26 nt, Homo sapiens
  source 1..26: mol_type = unassigned RNA
gtcccggggctgcgcgaggcacaggc

SEQ ID NO: 770 - RNA, 15 nt, Homo sapiens
  source 1..15: mol_type = unassigned RNA
ggcccggggggcggg

SEQ ID NO: 771 - RNA, 21 nt, Homo sapiens
  source 1..21: mol_type = unassigned RNA
aggaggcagtgggcgagcagg

SEQ ID NO: 772 - RNA, 21 nt, Homo sapiens
  source 1..21: mol_type = unassigned RNA
aggaggcagtgggcgagcagg

SEQ ID NO: 773 - RNA, 23 nt, Homo sapiens
  source 1..23: mol_type = unassigned RNA
acagcagggctggggattgcagt

SEQ ID NO: 774 - RNA, 19 nt, Homo sapiens
  source 1..19: mol_type = unassigned RNA
tgctgctcccagtcctgcc

SEQ ID NO: 775 - RNA, 19 nt, Homo sapiens
  source 1..19: mol_type = unassigned RNA
gctgggcgaggctggcatc

SEQ ID NO: 776 - RNA, 17 nt, Homo sapiens
  source 1..17: mol_type = unassigned RNA
gctgggcgaggctggca

SEQ ID NO: 777 - RNA, 18 nt, Homo sapiens
  source 1..18: mol_type = unassigned RNA
cagcggggctgggcgcgc

SEQ ID NO: 778 - RNA, 15 nt, Homo sapiens
  source 1..15: mol_type = unassigned RNA
cagcggggctgggcg

SEQ ID NO: 779 - RNA, 22 nt, Homo sapiens
  source 1..22: mol_type = unassigned RNA
caggcacgggagctcaggtgag

SEQ ID NO: 780 - RNA, 17 nt, Homo sapiens
  source 1..17: mol_type = unassigned RNA
caggcacgggagctcag

SEQ ID NO: 781 - RNA, 18 nt, Homo sapiens
  source 1..18: mol_type = unassigned RNA
ggcagcggcggcggcggc

SEQ ID NO: 782 - RNA, 15 nt, Homo sapiens
  source 1..15: mol_type = unassigned RNA
gctccccgcgccccc

SEQ ID NO: 783 - RNA, 20 nt, Homo sapiens
  source 1..20: mol_type = unassigned RNA
atcccaccactgccaccatt

SEQ ID NO: 784 - RNA, 15 nt, Homo sapiens
  source 1..15: mol_type = unassigned RNA
atcccaccactgcca

SEQ ID NO: 785 - RNA, 25 nt, Homo sapiens
  source 1..25: mol_type = unassigned RNA
gggggagccatgagataagagcacc

SEQ ID NO: 786 - RNA, 20 nt, Homo sapiens
  source 1..20: mol_type = unassigned RNA
tgggggagccatgagataag

SEQ ID NO: 787 - RNA, 17 nt, Homo sapiens
  source 1..17: mol_type = unassigned RNA
gatcccagcggtgcctc

SEQ ID NO: 788 - RNA, 15 nt, Homo sapiens
  source 1..15: mol_type = unassigned RNA
gatcccagcggtgcc

SEQ ID NO: 789 - RNA, 24 nt, Homo sapiens
  source 1..24: mol_type = unassigned RNA
agacacatttggagagggaacctc

SEQ ID NO: 790 - RNA, 16 nt, Homo sapiens
  source 1..16: mol_type = unassigned RNA
agacacatttggagag

SEQ ID NO: 791 - RNA, 30 nt, Homo sapiens
  source 1..30: mol_type = unassigned RNA
gctgggctgggacggacacccggcctccac

SEQ ID NO: 792 - RNA, 19 nt, Homo sapiens
  source 1..19: mol_type = unassigned RNA
gaggctgggctgggacgga

SEQ ID NO: 793 - RNA, 25 nt, Homo sapiens
  source 1..25: mol_type = unassigned RNA
tgggcaggggcttattgtaggagtc

SEQ ID NO: 794 - RNA, 19 nt, Homo sapiens
  source 1..19: mol_type = unassigned RNA
tgggcaggggcttattgta

SEQ ID NO: 795 - RNA, 21 nt, Homo sapiens
  source 1..21: mol_type = unassigned RNA
caactctgatctcttcatcta

SEQ ID NO: 796 - RNA, 20 nt, Homo sapiens
  source 1..20: mol_type = unassigned RNA
tctcttcatctaccccccag

SEQ ID NO: 797 - RNA, 23 nt, Homo sapiens
  source 1..23: mol_type = unassigned RNA
ctagtggaagaagatggcggaag

SEQ ID NO: 798 - RNA, 15 nt, Homo sapiens
  source 1..15: mol_type = unassigned RNA
tagtggaagaagatg

SEQ ID NO: 799 - RNA, 24 nt, Homo sapiens
  source 1..24: mol_type = unassigned RNA
ctgcggggacaggccagggcatct

SEQ ID NO: 800 - RNA, 20 nt, Homo sapiens
  source 1..20: mol_type = unassigned RNA
ctgcggggacaggccagggc

SEQ ID NO: 801 - RNA, 27 nt, Homo sapiens
  source 1..27: mol_type = unassigned RNA
agggccagaggagcctggagtggtcgg

SEQ ID NO: 802 - RNA, 23 nt, Homo sapiens
  source 1..23: mol_type = unassigned RNA
agggccagaggagcctggagtgg

SEQ ID NO: 803 - RNA, 18 nt, Homo sapiens
  source 1..18: mol_type = unassigned RNA
tctaggtggggagactga

SEQ ID NO: 804 - RNA, 16 nt, Homo sapiens
  source 1..16: mol_type = unassigned RNA
gtggggagactgacgg

SEQ ID NO: 805 - RNA, 23 nt, Homo sapiens
  source 1..23: mol_type = unassigned RNA
tcggctctgggtctgtggggagc

SEQ ID NO: 806 - RNA, 15 nt, Homo sapiens
  source 1..15: mol_type = unassigned RNA
gcccggatacctcag

SEQ ID NO: 807 - RNA, 30 nt, Homo sapiens
  source 1..30: mol_type = unassigned RNA
tgaggggcagagagcgagacttttctattt

SEQ ID NO: 808 - RNA, 15 nt, Homo sapiens
  source 1..15: mol_type = unassigned RNA
tgaggggcagagagc

SEQ ID NO: 809 - RNA, 25 nt, Homo sapiens
  source 1..25: mol_type = unassigned RNA
gggtggggatttgttgcattacttg

SEQ ID NO: 810 - RNA, 20 nt, Homo sapiens
  source 1..20: mol_type = unassigned RNA
gggtggggatttgttgcatt

SEQ ID NO: 811 - RNA, 27 nt, Homo sapiens
  source 1..27: mol_type = unassigned RNA
tagcagcacgtaaatattggcgttaag

SEQ ID NO: 812 - RNA, 15 nt, Homo sapiens
  source 1..15: mol_type = unassigned RNA
tagcagcacgtaaat

SEQ ID NO: 813 - RNA, 27 nt, Homo sapiens
  source 1..27: mol_type = unassigned RNA
aaaccgttaccattactgagtttagta

SEQ ID NO: 814 - RNA, 15 nt, Homo sapiens
  source 1..15: mol_type = unassigned RNA
gaaaccgttaccatt

SEQ ID NO: 815 - RNA, 22 nt, Homo sapiens
  source 1..22: mol_type = unassigned RNA
atatagggattggagccgtggc

SEQ ID NO: 816 - RNA, 20 nt, Homo sapiens
  source 1..20: mol_type = unassigned RNA
atatagggattggagccgtg

SEQ ID NO: 817 - RNA, 25 nt, Homo sapiens
  source 1..25: mol_type = unassigned RNA
tcctgtactgagctgccccgaggcc

SEQ ID NO: 818 - RNA, 15 nt, Homo sapiens
  source 1..15: mol_type = unassigned RNA
tcctgtactgagctg

SEQ ID NO: 819 - RNA, 26 nt, Homo sapiens
  source 1..26: mol_type = unassigned RNA
agggacgggacgcggtgcagtgttgt

SEQ ID NO: 820 - RNA, 15 nt, Homo sapiens
  source 1..15: mol_type = unassigned RNA
ggcgggcgggaggga

SEQ ID NO: 821 - RNA, 20 nt, Homo sapiens
  source 1..20: mol_type = unassigned RNA
ccccagggcgacgcggcggg

SEQ ID NO: 822 - RNA, 15 nt, Homo sapiens
  source 1..15: mol_type = unassigned RNA
cgcggcgggggcggc

SEQ ID NO: 823 - RNA, 15 nt, Homo sapiens
  source 1..15: mol_type = unassigned RNA
ggcggcgggcccggg

SEQ ID NO: 824 - RNA, 15 nt, Homo sapiens
  source 1..15: mol_type = unassigned RNA
ggcggcgggcccggg

SEQ ID NO: 825 - RNA, 25 nt, Homo sapiens
  source 1..25: mol_type = unassigned RNA
aaggcagggcccccgctccccgggc

SEQ ID NO: 826 - RNA, 15 nt, Homo sapiens
  source 1..15: mol_type = unassigned RNA
gtgtgttgaggaagg

SEQ ID NO: 827 - RNA, 23 nt, Homo sapiens
  source 1..23: mol_type = unassigned RNA
gagggccccccctcaatcctgtt

SEQ ID NO: 828 - RNA, 16 nt, Homo sapiens
  source 1..16: mol_type = unassigned RNA
agggccccccctcaat

SEQ ID NO: 829 - RNA, 26 nt, Homo sapiens
  source 1..26: mol_type = unassigned RNA
aaaatcacattgccagggattaccac

SEQ ID NO: 830 - RNA, 15 nt, Homo sapiens
  source 1..15: mol_type = unassigned RNA
aatcacattgccagg

SEQ ID NO: 831 - RNA, 28 nt, Homo sapiens
  source 1..28: mol_type = unassigned RNA
gtatggtattgcacttgtcccggcctgt

SEQ ID NO: 832 - RNA, 15 nt, Homo sapiens
  source 1..15: mol_type = unassigned RNA
tattgcacttgtccc

SEQ ID NO: 833 - RNA, 22 nt, Homo sapiens
  source 1..22: mol_type = unassigned RNA
gcacgactcagggcggagggaa

SEQ ID NO: 834 - RNA, 15 nt, Homo sapiens
  source 1..15: mol_type = unassigned RNA
agggcggagggaagt

SEQ ID NO: 835 - RNA, 18 nt, Homo sapiens
  source 1..18: mol_type = unassigned RNA
aaaaggaagggggaggag

SEQ ID NO: 836 - RNA, 16 nt, Homo sapiens
  source 1..16: mol_type = unassigned RNA
aaggaagggggaggag

SEQ ID NO: 837 - RNA, 24 nt, Homo sapiens
  source 1..24: mol_type = unassigned RNA
aatattgcactcgtcccggcctcc

SEQ ID NO: 838 - RNA, 15 nt, Homo sapiens
  source 1..15: mol_type = unassigned RNA
tattgcactcgtccc

SEQ ID NO: 839 - RNA, 21 nt, Homo sapiens
  source 1..21: mol_type = unassigned RNA
cccaggctggagcgagtgcag

SEQ ID NO: 840 - RNA, 15 nt, Homo sapiens
  source 1..15: mol_type = unassigned RNA
agctcactgcagcct

SEQ ID NO: 841 - RNA, 20 nt, Homo sapiens
  source 1..20: mol_type = unassigned RNA
tggctgttggagggggcagg

SEQ ID NO: 842 - RNA, 15 nt, Homo sapiens
  source 1..15: mol_type = unassigned RNA
ggagggggcaggctc

SEQ ID NO: 843 - RNA, 25 nt, Homo sapiens
  source 1..25: mol_type = unassigned RNA
tggcggcggtagttatgggcttctc

SEQ ID NO: 844 - RNA, 25 nt, Homo sapiens
  source 1..25: mol_type = unassigned RNA
tggcggcggtagttatgggcttctc

SEQ ID NO: 845 - RNA, 15 nt, Homo sapiens
  source 1..15: mol_type = unassigned RNA
tggcagagcgctgtc

SEQ ID NO: 846 - RNA, 15 nt, Homo sapiens
  source 1..15: mol_type = unassigned RNA
tggcagagcgctgtc

SEQ ID NO: 847 - RNA, 22 nt, Homo sapiens
  source 1..22: mol_type = unassigned RNA
cgcggcggggacggcgattggt

SEQ ID NO: 848 - RNA, 17 nt, Homo sapiens
  source 1..17: mol_type = unassigned RNA
cggcggggacggcgatt

SEQ ID NO: 849 - RNA, 22 nt, Homo sapiens
  source 1..22: mol_type = unassigned RNA
accccactcctggtaccatagt

SEQ ID NO: 850 - RNA, 15 nt, Homo sapiens
  source 1..15: mol_type = unassigned RNA
accccactcctggta

SEQ ID NO: 851 - RNA, 24 nt, Homo sapiens
  source 1..24: mol_type = unassigned RNA
ggaggccggggtggggcggggcgg

SEQ ID NO: 852 - RNA, 21 nt, Homo sapiens
  source 1..21: mol_type = unassigned RNA
tggggcggggcaggtccctgc

SEQ ID NO: 853 - RNA, 18 nt, Homo sapiens
  source 1..18: mol_type = unassigned RNA
gggtgcgggccggcgggg

SEQ ID NO: 854 - RNA, 18 nt, Homo sapiens
  source 1..18: mol_type = unassigned RNA
agggggcgggctccggcg

SEQ ID NO: 855 - RNA, 22 nt, Homo sapiens
  source 1..22: mol_type = unassigned RNA
ggggggtgtggagccagggggc

SEQ ID NO: 856 - RNA, 25 nt, Homo sapiens
  source 1..25: mol_type = unassigned RNA
aagggaggaggagcggaggggccct

SEQ ID NO: 857 - RNA, 64 nt, Homo sapiens
  source 1..64: mol_type = unassigned RNA
ccccgggcccggcgttccctccccttccgtgcgccagtggaggccggggtggggcggggcgggg

SEQ ID NO: 858 - RNA, 64 nt, Homo sapiens; identical to SEQ ID NO: 857
  source 1..64: mol_type = unassigned RNA

SEQ ID NO: 859 - RNA, 66 nt, Homo sapiens
  source 1..66: mol_type = unassigned RNA
ccgagtggggcggggcaggtccctgcagggactgtgacactgaaggacctgcaccttcgcccacag

SEQ ID NO: 860 - RNA, 54 nt, Homo sapiens
  source 1..54: mol_type = unassigned RNA
acgcgggtgcgggccggcggggtagaagccacccggcccggcccggcccggcga

SEQ ID NO: 861 - RNA, 62 nt, Homo sapiens
  source 1..62: mol_type = unassigned RNA
ggtagggggcgggctccggcgctgggaccccactagggtggcgccttggccccgccccgccc

SEQ ID NO: 862 - RNA, 71 nt, Homo sapiens
  source 1..71: mol_type = unassigned RNA
atggaggggggtgtggagccagggggcccaggtctacagcttctccccgctccctgcccccatactcccag

SEQ ID NO: 863 - RNA, 74 nt, Homo sapiens
  source 1..74: mol_type = unassigned RNA
gggaggaagaagggaggaggagcggaggggcccttgtcttcccagagcctctcccttcctcccctccccctccc

SEQ ID NO: 864 - RNA, 16 nt, Homo sapiens
  source 1..16: mol_type = unassigned RNA
cgggcccggcgttccc

SEQ ID NO: 865 - RNA, 15 nt, Homo sapiens
  source 1..15: mol_type = unassigned RNA
ccgggcccggcgttc

SEQ ID NO: 866 - RNA, 19 nt, Homo sapiens
  source 1..19: mol_type = unassigned RNA
gggtgcgggccggcggggt

SEQ ID NO: 867 - RNA, 15 nt, Homo sapiens
  source 1..15: mol_type = unassigned RNA
tgcgggccggcgggg

SEQ ID NO: 868 - RNA, 19 nt, Homo sapiens
  source 1..19: mol_type = unassigned RNA
agggggcgggctccggcgc

SEQ ID NO: 869 - RNA, 15 nt, Homo sapiens
  source 1..15: mol_type = unassigned RNA
gtagggggcgggctc

SEQ ID NO: 870 - RNA, 23 nt, Homo sapiens
  source 1..23: mol_type = unassigned RNA
aagggaggaggagcggaggggcc

SEQ ID NO: 871 - RNA, 15 nt, Homo sapiens
  source 1..15: mol_type = unassigned RNA
gggaggaggagcgga